SEQUENCE LISTING (1318 sequences)

SEQ ID NO: 1 - protein, 119 aa, synthetic construct
QVQLQQSGPELVKPGTSVKISCKASGYSFTTYYIHWVKQRPGQGLEWIGWFFPGSGNIKYNEKFKGKATLTADTSSSTAYMQLSSLTSEESAVYFCAGSYYSYDVLDYWGHGTTLTVSS

SEQ ID NO: 2 - protein, 107 aa, synthetic construct
DILMTQSQKFMSTSLGDRVSVSCKASQNVGINVVWHQQKPGQSPKALIYSSSHRYSGVPDRFTGSGSGTDFTLTINNVQSEDLAEYFCQQFKSYPLTFGAGTKLELK

SEQ ID NO: 3 - protein, 10 aa, synthetic construct
GYSFTTYYIH

SEQ ID NO: 4 - protein, 17 aa, synthetic construct
WFFPGSGNIKYNEKFKG

SEQ ID NO: 5 - protein, 10 aa, synthetic construct
SYYSYDVLDY

SEQ ID NO: 6 - protein, 11 aa, synthetic construct
KASQNVGINVV

SEQ ID NO: 7 - protein, 7 aa, synthetic construct
SSSHRYS

SEQ ID NO: 8 - protein, 9 aa, synthetic construct
QQFKSYPLT

SEQ ID NO: 9 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 10 - protein, 107 aa, synthetic construct
DIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 11 - protein, 107 aa, synthetic construct
DIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIK

SEQ ID NO: 12 - DNA, 357 bp, synthetic construct
  source 1..357: mol_type = other DNA
caggtgcagctggttcagtctggcgccgaagtgaagaaacctggctcctccgtgaaggtgtcctgcaaggcttccggctactccttcaccacctactacatccactgggtccgacaggcccctggacaaggattggaatggatgggctggttcttccccggctccggcaacatcaagtacaacgagaagttcaagggccgcgtgaccatcaccgccgacacctctacctctaccgcctacatggaactgtccagcctgagatctgaggacaccgccgtgtactactgcgccggctcctactactcttacgacgtgctggattactggggccagggcaccacagtgacagtgtcctct

SEQ ID NO: 13 - DNA, 321 bp, synthetic construct
  source 1..321: mol_type = other DNA
gacatccagatgacccagtctccatccttcctgtccgcctctgtgggcgacagagtgaccatcacatgcaaggcctctcagaacgtgggcatcaacgtcgtgtggcaccagcagaagcctggcaaggctcctaaggctctgatctactcctccagccaccggtactctggcgtgccctctagattttccggctctggctctggcaccgagtttaccctgacaatctccagcctgcagcctgaggacttcgccacctacttttgccagcagttcaagagctaccctctgacctttggccagggcaccaagctggaaatcaag

SEQ ID NO: 14 - DNA, 321 bp, synthetic construct
  source 1..321: mol_type = other DNA
gacatccagatgacccagtctccatcctctctgtccgcctctgtgggcgacagagtgaccatcacatgcaaggcctctcagaacgtgggcatcaacgtcgtgtggcaccagcagaaacctggcaaggtgcccaaggctctgatctactcctccagccacagatactccggcgtgccctctagattctccggctctggctctggcaccgactttaccctgacaatctccagcctgcagcctgaggacgtggccacctacttttgccagcagttcaagagctaccctctgacctttggccagggcaccaagctggaaatcaag

SEQ ID NO: 15 - protein, 7 aa, synthetic construct
GYSFTTY

SEQ ID NO: 16 - protein, 6 aa, synthetic construct
FPGSGN

SEQ ID NO: 17 - protein, 592 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 18 - protein, 492 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 19 - protein, 479 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 20 - protein, 469 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 234 aa, synthetic construct
METDTLLLWVLLLWVPGSTGDIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 246 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 23 - protein, 234 aa, synthetic construct
METDTLLLWVLLLWVPGSTGDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 107 aa, synthetic construct
DIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 25 - protein, 119 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 26 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 27 - protein, 119 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 28 - protein, 246 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 29 - protein, 107 aa, synthetic construct
DIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 30 - protein, 119 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSS

SEQ ID NO: 31 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 32 - protein, 107 aa, synthetic construct
DIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 33 - protein, 119 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 34 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 35 - protein, 107 aa, synthetic construct
DIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 36 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSS

SEQ ID NO: 37 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 38 - protein, 119 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 39 - protein, 107 aa, Homo sapiens [sequence omitted]

SEQ ID NO: 40 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43-44 - skipped (2 entries)

SEQ ID NO: 45 - protein, 5 aa, synthetic construct
TYYIH

SEQ ID NO: 46 - protein, 246 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 47 - protein, 119 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSS

SEQ ID NO: 48 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 49 - protein, 107 aa, synthetic construct
DIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 50 - protein, 119 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 51 - protein, 247 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIKS

SEQ ID NO: 52 - protein, 107 aa, synthetic construct
DIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 53 - protein, 119 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSS

SEQ ID NO: 54 - protein, 246 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 55 - protein, 107 aa, synthetic construct; identical to SEQ ID NO: 52

SEQ ID NO: 56 - protein, 119 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSS

SEQ ID NO: 57 - protein, 246 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 58 - protein, 107 aa, synthetic construct
DIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 59 - protein, 119 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 60 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 61 - protein, 246 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 62 - protein, 107 aa, synthetic construct
DIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 63 - protein, 119 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 64 - protein, 246 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 65 - protein, 119 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSS

SEQ ID NO: 66 - protein, 246 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 67 - protein, 119 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 68 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 69 - protein, 246 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 70 - protein, 119 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 71 - protein, 246 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 72 - protein, 119 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 73 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 74 - protein, 353 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 75 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 76 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 77 - protein, 246 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 78 - protein, 119 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 79 - protein, 246 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 80 - protein, 119 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 81 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 82 - protein, 107 aa, synthetic construct
DIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 83 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 84 - protein, 246 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 85 - protein, 119 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 86 - protein, 246 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 87 - protein, 107 aa, synthetic construct
DIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 88 - protein, 119 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSS

SEQ ID NO: 89 - protein, 246 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 90 - protein, 119 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 91 - protein, 246 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 92 - protein, 119 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 93 - protein, 246 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 94 - protein, 119 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 95 - protein, 246 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 96 - protein, 107 aa, synthetic construct
DIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 97 - protein, 119 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 98 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 99 - protein, 246 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 100 - protein, 119 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSS

SEQ ID NO: 101 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 102 - protein, 107 aa, synthetic construct; identical to SEQ ID NO: 82

SEQ ID NO: 103 - protein, 246 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 104 - protein, 119 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 105 - protein, 246 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 106 - protein, 119 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 107 - protein, 246 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 108 - protein, 119 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 109 - protein, 246 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 110 - protein, 107 aa, synthetic construct
DIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 111 - protein, 119 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 112 - protein, 246 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 113 - protein, 119 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSS

SEQ ID NO: 114 - protein, 246 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 115 - protein, 119 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 116 - protein, 246 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 117 - protein, 119 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSS

SEQ ID NO: 118 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 119 - protein, 246 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 120 - protein, 119 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 121 - protein, 246 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 122 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 123 - protein, 119 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 124 - protein, 246 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 125 - protein, 119 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 126 - protein, 246 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVVWYQQKPGKAPKALIYSSSHRYKGVPSRFSGSGSGTEFTLTISSLQPEDFATYFCQQFKSYPLTFGQGTKLEIK

SEQ ID NO: 127 - protein, 119 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 128 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 129 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSS

SEQ ID NO: 130 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 131 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSS

SEQ ID NO: 132 - protein, 246 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 133 - protein, 119 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSS

SEQ ID NO: 134 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 135 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSS

SEQ ID NO: 136 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 137 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSS

SEQ ID NO: 138 - protein, 246 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 139 - protein, 119 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSS

SEQ ID NO: 140 - protein, 246 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 141 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSS

SEQ ID NO: 142 - protein, 246 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 143 - protein, 119 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 144 - protein, 246 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 145 - protein, 119 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 146 - protein, 246 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 147 - protein, 119 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 148 - protein, 106 aa, synthetic construct
QSVLTQPPSVSEAPRQRVTISCKASQNVGINVVWHQQLPGKAPKALIYSSSHRYSGVSDRFSGSGSGTSFSLAISGLQSEDEADYFCQQFKSYPLTFGTGTKVTVL

SEQ ID NO: 149 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 150 - protein, 246 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 151 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 152 - protein, 246 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 153 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 154 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 155 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 156 - protein, 246 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 157 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 158 - protein, 246 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 159 - protein, 246 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 160 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 161 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 162 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSS

SEQ ID NO: 163 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSS

SEQ ID NO: 164 - protein, 10 aa, synthetic construct
GHDFRLTYIH

SEQ ID NO: 165 - protein, 17 aa, synthetic construct
RVSAGSGNVKYNEKFKG

SEQ ID NO: 166 - protein, 11 aa, synthetic construct
KASQNVGNRVV

SEQ ID NO: 167 - protein, 7 aa, synthetic construct
SSSHRYK

SEQ ID NO: 168 - protein, 119 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSS

SEQ ID NO: 169 - protein, 107 aa, synthetic construct
DIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 170 - protein, 17 aa, synthetic construct
RIYAGSGNVKYNEKFKG

SEQ ID NO: 171 - protein, 11 aa, synthetic construct
KASQNVADRVV

SEQ ID NO: 172 - protein, 119 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSS

SEQ ID NO: 173 - protein, 107 aa, synthetic construct
DIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 174 - protein, 10 aa, synthetic construct
GHDFKLTYIH

SEQ ID NO: 175 - protein, 17 aa, synthetic construct
RVSAGSGNTKYNEKFKG

SEQ ID NO: 176 - protein, 11 aa, synthetic construct
KASQNVDNRVA

SEQ ID NO: 177 - protein, 119 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 178 - protein, 17 aa, synthetic construct
RVYAGSGNTKYNEKFKG

SEQ ID NO: 179 - protein, 11 aa, synthetic construct
KASQNVEDRVV

SEQ ID NO: 180 - protein, 119 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSS

SEQ ID NO: 181 - protein, 17 aa, synthetic construct
RISAGSGNTKYNEKFKG

SEQ ID NO: 182 - protein, 11 aa, synthetic construct
KASQNVGDRVV

SEQ ID NO: 183 - protein, 119 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSS

SEQ ID NO: 184 - protein, 107 aa, synthetic construct
DIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 185 - protein, 119 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSS

SEQ ID NO: 186 - protein, 11 aa, synthetic construct
RASQNVDNRLG

SEQ ID NO: 187 - protein, 107 aa, synthetic construct
DIQMTQSPSFLSASVGDRVTITCRASQNVDNRLGWHQQKPGKAPKALIYSSSHRYKGVPSRFSGSGSGTEFTLTISSLQPEDFATYFCQQFKSYPLTFGQGTKLEIK

SEQ ID NO: 188 - protein, 7 aa, synthetic construct
AASSLQK

SEQ ID NO: 189 - protein, 107 aa, synthetic construct
DIQMTQSPSFLSASVGDRVTITCKASQNVDNRVAWHQQKPGKAPKALIYAASSLQKGVPSRFSGSGSGTEFTLTISSLQPEDFATYFCQQFKSYPLTFGQGTKLEIK

SEQ ID NO: 190 - protein, 9 aa, synthetic construct
LQHNSYPLT

SEQ ID NO: 191 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCLQHNSYPLTFGQGTKLEIK

SEQ ID NO: 192 - protein, 17 aa, synthetic construct
RVSAGSGNVNYAQKFQG

SEQ ID NO: 193 - protein, 119 aa, synthetic construct
QVQLVQSGAEVKKPGSSVKVSCKASGHDFKLTYIHWVRQAPGQGLEWMGRVSAGSGNVNYAQKFQGRVTITADTSTSTAYMELSSLRSEDTAVYYCAVSYYSYDVLDYWGQGTTVTVSS

SEQ ID NO: 194 - protein, 119 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 195 - protein, 113 aa, synthetic construct
QVQLVQSGAEVKKPGSSVKVSCKASGFNIKDTHWVRQAPGQGLEWMGTDPASGDTRVTITADTSTSTAYMELSSLRSEDTAVYYCAVCAHYYRDDVNYAMDYWGQGTTVTVSS

SEQ ID NO: 196 - protein, 98 aa, synthetic construct
DIQMTQSPSFLSASVGDRVTITCQNVGSNWHQQKPGKAPKALIYSSSGVPSRFSGSGSGTEFTLTISSLQPEDFATYFCQQYNTYPYTFGQGTKLEIK

SEQ ID NO: 197 - protein, 8 aa, synthetic construct
GFNIKDTH

SEQ ID NO: 198 - protein, 8 aa, synthetic construct
TDPASGDT

SEQ ID NO: 199 - protein, 15 aa, synthetic construct
CAHYYRDDVNYAMDY

SEQ ID NO: 200 - protein, 6 aa, synthetic construct
QNVGSN

SEQ ID NO: 201 - skipped

SEQ ID NO: 202 - protein, 9 aa, synthetic construct
QQYNTYPYT

SEQ ID NO: 203 - protein, 107 aa, Homo sapiens [sequence omitted]

SEQ ID NO: 204 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 334 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPEA

SEQ ID NO: 206 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 208-227 - skipped (20 entries)

SEQ ID NO: 228 - protein, 449 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 229 - protein, 5 aa, synthetic construct
LTYIH

SEQ ID NO: 230-233 - skipped (4 entries)

SEQ ID NO: 234 - protein, 7 aa, synthetic construct
FRLTYIH

SEQ ID NO: 235 - protein, 448 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 77 aa, synthetic construct
ITCPPPMSVEHADIWVKSYSLYSRERYICNSGFKRKAGTSSLTECVLNKATNVAHWTTPSLKCIRDPALVHQRPAPP

SEQ ID NO: 237 - protein, 10 aa, synthetic construct
GGGGSGGGGS

SEQ ID NO: 238 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 239 - protein, 214 aa, synthetic construct
DIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 351 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 370 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 243 - protein, 20 aa, synthetic construct
SGGSGGGGSGGGSGGGGSGG

SEQ ID NO: 244 - protein, 452 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGKEPEA

SEQ ID NO: 245 - protein, 448 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 246 - protein, 192 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSS

SEQ ID NO: 247 - protein, 10 aa, synthetic construct
GYTFSNYWIE

SEQ ID NO: 248 - protein, 17 aa, synthetic construct
EILPGAGPTNYNEKFKG

SEQ ID NO: 249 - protein, 10 aa, synthetic construct
TDYDYDWFAY

SEQ ID NO: 250 - protein, 260 aa, synthetic construct
TWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKEPEA

SEQ ID NO: 251 - protein, 368 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 252 - protein, 113 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIK

SEQ ID NO: 253 - protein, 11 aa, synthetic construct
KSSQSLLYSGN

SEQ ID NO: 254 - protein, 8 aa, synthetic construct
PKLLIYWA

SEQ ID NO: 255 - protein, 9 aa, synthetic construct
QQYYGYPRT

SEQ ID NO: 256 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 257 - protein, 452 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKEPEA

SEQ ID NO: 258 - protein, 15 aa, synthetic construct
GGGGSGGGGSGGGGS

SEQ ID NO: 259 - protein, 5 aa, synthetic construct
GGGGS

SEQ ID NO: 260 - protein, 14 aa, synthetic construct
AEAAAKEAAAKAAA

SEQ ID NO: 261 - skipped

SEQ ID NO: 262 - protein, 24 aa, synthetic construct
AEAAAKEAAAKEAAAKEAAAKAAA

SEQ ID NO: 263 - protein, 29 aa, synthetic construct
AEAAAKEAAAKEAAAKEAAAKEAAAKAAA

SEQ ID NO: 264 - protein, 256 aa, synthetic construct
TWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 265 - protein, 4 aa, synthetic construct
EPEA

SEQ ID NO: 266 - protein, 383 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGGGSHHHHHHHH

SEQ ID NO: 267 - protein, 370 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 268 - protein, 133 aa, synthetic construct [sequence omitted]

SEQ ID NO: 269 - protein, 227 aa, synthetic construct
DKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 270 - protein, 13 aa, synthetic construct
GGGGSHHHHHHHH

SEQ ID NO: 271 - protein, 220 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 272 - protein, 389 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 274 - protein, 133 aa, synthetic construct [sequence omitted]

SEQ ID NO: 275 - protein, 755 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 277 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPC

SEQ ID NO: 278 - protein, 16 aa, synthetic construct
SGGGGSGGGGSGGGGS

SEQ ID NO: 279 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 280-287 - skipped (8 entries)

SEQ ID NO: 288 - protein, 370 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 289 - protein, 447 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 290-291 - skipped (2 entries)

SEQ ID NO: 292 - protein, 449 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 294 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 295 - protein, 78 aa, synthetic construct
ITCPPPMSVEHADIWVKSYSLYSRERYICNSGFKRKAGTSSLTECVLNKATNVAHWTTPSLKCIRDPALVHQRPAPPS

SEQ ID NO: 296 - protein, 20 aa, synthetic construct
GGSGGGGSGGGSGGGGSGGG

SEQ ID NO: 297 - protein, 212 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 298 - protein, 214 aa, synthetic construct
DIQMTQSPSFLSASVGDRVTITCKASQNVADRVVWHQQKPGKAPKALIYAASYLQSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 7 aa, synthetic construct
AASYLQS

SEQ ID NO: 300 - protein, 107 aa, synthetic construct
DIQMTQSPSFLSASVGDRVTITCKASQNVADRVVWHQQKPGKAPKALIYAASYLQSGVPSRFSGSGSGTEFTLTISSLQPEDFATYFCQQFKSYPLTFGQGTKLEIK

SEQ ID NO: 301 - protein, 27 aa, synthetic construct
  VARIANT 7..26: note = EAAAK repeats may be deleted
AEAAAKEAAAKEAAAKEAAAKEAAAKA

SEQ ID NO: 302 - skipped

SEQ ID NO: 303 - protein, 107 aa, synthetic construct
DIQMTQSPSFLSASVGDRVTITCKASQNVADRVVWHQQKPGKAPKALIFAASYLQSGVPSRFSGSGSGTEFTLTISSLQPEDFATYFCQQFKSYPLTFGQGTKLEIK

SEQ ID NO: 304 - protein, 449 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVAMFINTSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 17 aa, synthetic construct
DVPSGPGGGGGSGGGGS

SEQ ID NO: 306 - protein, 16 aa, synthetic construct
GGGGSGGGGSGGGGGS

SEQ ID NO: 307 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVAMFINTS

SEQ ID NO: 308 - protein, 212 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVAMFINTS

SEQ ID NO: 309 - protein, 449 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVEQLIILANNSLSSNGNVTESGCKECEELEEKNIKEFLQSFVHIVQMFINTSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 114 aa, synthetic construct
NWVNVISDLKKIEDLIQSMHIDATLYTESDVHPSCKVTAMKCFLLELQVISLESGDASIHDTVEQLIILANNSLSSNGNVTESGCKECEELEEKNIKEFLQSFVHIVQMFINTS

SEQ ID NO: 311 - protein, 212 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVEQLIILANNSLSSNGNVTESGCKECEELEEKNIKEFLQSFVHIVQMFINTS

SEQ ID NO: 312 - skipped

SEQ ID NO: 313 - protein, 166 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 314 - protein, 5 aa, synthetic construct
SYAMS

SEQ ID NO: 315 - protein, 17 aa, synthetic construct
HISNGGDYIYYADTVKG

SEQ ID NO: 316 - protein, 12 aa, synthetic construct
PSYYSDPWFFDV

SEQ ID NO: 317 - protein, 7 aa, synthetic construct
GFTFRSY

SEQ ID NO: 318 - protein, 6 aa, synthetic construct
SNGGDY

SEQ ID NO: 319 - protein, 10 aa, synthetic construct
GFTFRSYAMS

SEQ ID NO: 320 - protein, 10 aa, synthetic construct
SAGSSVSFMH

SEQ ID NO: 321 - protein, 7 aa, synthetic construct
DTSKLAS

SEQ ID NO: 322 - protein, 9 aa, synthetic construct
LQGSGFPLT

SEQ ID NO: 323 - protein, 6 aa, synthetic construct
GSSVSF

SEQ ID NO: 324 - protein, 106 aa, synthetic construct
ENVLTQSPAIMSASPGEKVTMTCSAGSSVSFMHWYQQKSSTSPKLWIYDTSKLASGVPGRFSGSGSGNSFSLTISSMEAEDVAIYYCLQGSGFPLTFGSGTKLEIK

SEQ ID NO: 325 - protein, 121 aa, synthetic construct
DVKLVESGEGLVKPGGSLKLSCAASGFTFRSYAMSWVRQTPEKRLEWVAHISNGGDYIYYADTVKGRFTISRDNARNTLYLQMSSLKSEDTAMYYCTRPSYYSDPWFFDVWGTGTTVTVSS

SEQ ID NO: 326 - protein, 121 aa, synthetic construct
EVQLVESGGGLVQPGGSLRLSCAASGFTFRSYAMSWVRQAPGKGLEWVAHISNGGDYIYYADTVKGRFTISRDNAKNSLYLQMNSLRAEDTAVYYCTRPSYYSDPWFFDVWGQGTTVTVSS

SEQ ID NO: 327 - protein, 121 aa, synthetic construct
QVQLVESGGGVVQPGRSLRLSCAASGFTFRSYAMSWVRQAPGKGLEWVAHISNGGDYIYYADTVKGRFTISRDNSKNTLYLQMSSLRAEDTAVYYCTRPSYYSDPWFFDVWGQGTTVTVSS

SEQ ID NO: 328 - protein, 121 aa, synthetic construct
EVQLVESGGGLVQPGGSLRLSCAASGFTFRSYAMSWVRQAPGKGLEWVAHISNGGDYIYYADTVKGRFTISRDNSKNTLYLQMNSLRAEDTAVYYCTRPSYYSDPWFFDVWGQGTTVTVSS

SEQ ID NO: 329 - protein, 121 aa, synthetic construct
QVQLVQSGSELKKPGASVKVSCKASGFTFRSYAMSWVRQAPGQGLEWVAHISNGGDYIYYADTVKGRFVISRDNSVNTLYLQISSLKAEDTAVYYCTRPSYYSDPWFFDVWGQGTTVTVSS

SEQ ID NO: 330 - protein, 121 aa, synthetic construct
QVQLVQSGAEVKKPGASVKVSCKASGFTFRSYAMSWVRQAPGQRLEWVAHISNGGDYIYYADTVKGRFTITRDNSANTLYMELSSLRSEDTAVYYCTRPSYYSDPWFFDVWGQGTTVTVSS

SEQ ID NO: 331 - protein, 106 aa, synthetic construct
ENVLTQSPATLSLSPGERATLSCSAGSSVSFMHWYQQKPGQAPKLLIYDTSKLASGIPARFSGSGSGNDFTLTISSLEPEDFAVYYCLQGSGFPLTFGQGTKLEIK

SEQ ID NO: 332 - protein, 106 aa, synthetic construct
ENVLTQSPDFQSVTPKEKVTITCSAGSSVSFMHWYQQKPDQSPKLLIYDTSKLASGVPSRFSGSGSGNDFTLTINSLEAEDAATYYCLQGSGFPLTFGQGTKLEIK

SEQ ID NO: 333 - protein, 106 aa, synthetic construct
DNQLTQSPSSLSASVGDRVTITCSAGSSVSFMHWYQQKPGKVPKLLIYDTSKLASGVPSRFSGSGSGNDFTLTISSLQPEDVATYYCLQGSGFPLTFGQGTKLEIK

SEQ ID NO: 334 - protein, 106 aa, synthetic construct
ANQLTQSPSSLSASVGDRVTITCSAGSSVSFMHWYQQKPGKAPKLLIYDTSKLASGVPSRFSGSGSGNDFTLTISSLQPEDFATYYCLQGSGFPLTFGQGTKLEIK

SEQ ID NO: 335 - protein, 106 aa, synthetic construct
DNVLTQSPDSLAVSLGERATINCSAGSSVSFMHWYQQKPGQPPKLLIYDTSKLASGVPDRFSGSGSGNDFTLTISSLQAEDVAVYYCLQGSGFPLTFGQGTKLEIK

SEQ ID NO: 336 - protein, 449 aa, synthetic construct
QVQLVQSGAEVKKPGASVKVSCKASGFNIKSAYMHWVRQAPGQEPGCMGRIDPATGKTKYAPKFQARVTMTADTSINTAYTELSSLRSEDTATYYCARSLNWDYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 337 - protein, 218 aa, synthetic construct
EIVLTQSPATLSLSPGERATLSCRASKSVSILGTHLIHWYQQKPGLAPRLLIYAASNLESGIPDRFSGSGSETDFTLTISRLEPEDFAVYFCQQSIEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 370 aa, synthetic construct; identical to SEQ ID NO: 241

SEQ ID NO: 339 - protein, 10 aa, synthetic construct
GFNIKSAYMH

SEQ ID NO: 340 - protein, 17 aa, synthetic construct
RIDPATGKTKYAPKFQA

SEQ ID NO: 341 - protein, 10 aa, synthetic construct
SLNWDYGLDY

SEQ ID NO: 342 - protein, 15 aa, synthetic construct
RASKSVSILGTHLIH

SEQ ID NO: 343 - protein, 7 aa, synthetic construct
AASNLES

SEQ ID NO: 344 - protein, 9 aa, synthetic construct
QQSIEDPWT

SEQ ID NO: 345 - protein, 119 aa, synthetic construct
QVQLVQSGAEVKKPGASVKVSCKASGFNIKSAYMHWVRQAPGQEPGCMGRIDPATGKTKYAPKFQARVTMTADTSINTAYTELSSLRSEDTATYYCARSLNWDYGLDYWGQGTLVTVSS

SEQ ID NO: 346 - protein, 111 aa, synthetic construct
EIVLTQSPATLSLSPGERATLSCRASKSVSILGTHLIHWYQQKPGLAPRLLIYAASNLESGIPDRFSGSGSETDFTLTISRLEPEDFAVYFCQQSIEDPWTFGGGTKVEIK

SEQ ID NO: 347 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 348 - protein, 449 aa, synthetic construct
QVQLVQSGAEVKKPGASVKVSCKASGFNIKSAYMHWVRQAPGQGLEWMGRIDPATGKTKYAPKFQARVTMTADTSTNTAYMELSSLRSEDTAVYYCARSLNWDYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 349 - protein, 218 aa, synthetic construct
EIVLTQSPGTLSLSPGERATLSCRASKSVSILGTHLIHWYQQKPGQAPRLLIYAASNLESGIPDRFSGSGSETDFTLTISRLEPEDFAVYFCQQSIEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 350 - protein, 119 aa, synthetic construct
QVQLVQSGAEVKKPGASVKVSCKASGFNIKSAYMHWVRQAPGQGLEWMGRIDPATGKTKYAPKFQARVTMTADTSTNTAYMELSSLRSEDTAVYYCARSLNWDYGLDYWGQGTLVTVSS

SEQ ID NO: 351 - protein, 111 aa, synthetic construct
EIVLTQSPGTLSLSPGERATLSCRASKSVSILGTHLIHWYQQKPGQAPRLLIYAASNLESGIPDRFSGSGSETDFTLTISRLEPEDFAVYFCQQSIEDPWTFGGGTKVEIK

SEQ ID NO: 352 - protein, 368 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 353 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 354 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 355 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 356 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 357 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIK

SEQ ID NO: 358 - protein, 116 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARNGYLGDYWGQGTLVTVSS

SEQ ID NO: 359 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQAMQIPTFGQGTKVEIK

SEQ ID NO: 360 - protein, 120 aa, synthetic construct
QVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSS

SEQ ID NO: 361 - protein, 108 aa, synthetic construct
DIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELKR

SEQ ID NO: 362 - protein, 123 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSS

SEQ ID NO: 363 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 364 - protein, 11 aa, synthetic construct
KASQNVGDRVA

SEQ ID NO: 365 - protein, 17 aa, synthetic construct
RIYAGSGNTKYNEKFKG

SEQ ID NO: 366-409 - skipped (44 entries)

SEQ ID NO: 410 - protein, 114 aa, Homo sapiens; identical to SEQ ID NO: 238

SEQ ID NO: 411 - protein, 114 aa, Homo sapiens
NWVNVISDLKKIEDLIQSMHIDATLYTESDVHPSCKVTAMKCFLLELQVISLASGDASIHDTVENLIILANNSLSSNGAVTESGCKECEELEEKNIKEFLQSFVHIVQMFINTS

SEQ ID NO: 412 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 413 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 414 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 415 - protein, 177 aa, Homo sapiens
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 416 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 417 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 418 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 419 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 420 - protein, 189 aa, Homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 421 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 422 - protein, 180 aa, Homo sapiens
MARSFSLLMVVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTN

SEQ ID NO: 423 - protein, 189 aa, Homo sapiens
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 424 - protein, 189 aa, Homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 425 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 426 - protein, 189 aa, Homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 427 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 428 - protein, 107 aa, Homo sapiens [sequence omitted]

SEQ ID NO: 429 - protein, 107 aa, Homo sapiens [sequence omitted]

SEQ ID NO: 430 - protein, 326 aa, synthetic construct; identical to SEQ ID NO: 40

SEQ ID NO: 431 - protein, 330 aa, synthetic construct; identical to SEQ ID NO: 41

SEQ ID NO: 432 - protein, 330 aa, synthetic construct; identical to SEQ ID NO: 42

SEQ ID NO: 433 - protein, 453 aa, synthetic construct; identical to SEQ ID NO: 73

SEQ ID NO: 434 - protein, 353 aa, synthetic construct; identical to SEQ ID NO: 74

SEQ ID NO: 435 - protein, 340 aa, synthetic construct; identical to SEQ ID NO: 75

SEQ ID NO: 436 - protein, 159 aa, Homo sapiens; identical to SEQ ID NO: 76

SEQ ID NO: 437 - protein, 330 aa, Homo sapiens; identical to SEQ ID NO: 204

SEQ ID NO: 438 - protein, 334 aa, Homo sapiens; identical to SEQ ID NO: 205

SEQ ID NO: 439 - protein, 330 aa, Homo sapiens; identical to SEQ ID NO: 41

SEQ ID NO: 440 - protein, 227 aa, Homo sapiens; identical to SEQ ID NO: 206

SEQ ID NO: 441 - protein, 114 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVS

SEQ ID NO: 442 - protein, 114 aa, Homo sapiens
MTIRLLCYVGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSE

SEQ ID NO: 443 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 444 - protein, 133 aa, Homo sapiens; identical to SEQ ID NO: 242

SEQ ID NO: 445 - protein, 133 aa, Homo sapiens
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 446 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 447 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 448 - protein, 518 aa, Homo sapiens; identical to SEQ ID NO: 276

SEQ ID NO: 449 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 450 - protein, 24 aa, synthetic construct
AEAAAKEAAAKEAAAKEAAAKAAA

SEQ ID NO: 451 - protein, 29 aa, synthetic construct
AEAAAKEAAAKEAAAKEAAAKEAAAKAAA

SEQ ID NO: 452 - protein, 120 aa, synthetic construct
QVQMKQSGPGLVQPSQSLSITCTVSGFSLTSYGVHWVRQSPGKGLEWLGVIWSGGSTDYNAAFISRLSISKDNSKSQVFFKMNSLQADDTAIYYCVRNFYDGYYEAMDYWGQGTSVTVSS

SEQ ID NO: 453 - protein, 10 aa, synthetic construct
GFSLTSYGVH

SEQ ID NO: 454 - protein, 16 aa, synthetic construct
VIWSGGSTDYNAAFIS

SEQ ID NO: 455 - protein, 12 aa, synthetic construct
NFYDGYYEAMDY

SEQ ID NO: 456 - protein, 107 aa, synthetic construct
DIQMTQSPASLSASVGETVTITCRASENIYSYLAWYQQKQGKSPQLLVYNAKTLAEGVPSRFSGSGSGTQFSLKINSLQPEDFGSYYCQHHYGTPRTFGGGTKVEIK

SEQ ID NO: 457 - protein, 11 aa, synthetic construct
RASENIYSYLA

SEQ ID NO: 458 - protein, 7 aa, synthetic construct
NAKTLAE

SEQ ID NO: 459 - protein, 9 aa, synthetic construct
QHHYGTPRT

SEQ ID NO: 460 - protein, 116 aa, synthetic construct
QVQLQQSGPEVVRPGVSVKISCKGSGYRFTDSAMHWVKQSHAKSLEWIGVISSYNGNTNYNQKFKGKATMTVDKSSSTAYMELARMTSEDSAIYYCARSRDAMDYWGQGTSVTVSS

SEQ ID NO: 461 - protein, 10 aa, synthetic construct
GYRFTDSAMH

SEQ ID NO: 462 - protein, 17 aa, synthetic construct
VISSYNGNTNYNQKFKG

SEQ ID NO: 463 - protein, 7 aa, synthetic construct
SRDAMDY

SEQ ID NO: 464 - protein, 107 aa, synthetic construct
DIKMTQSPSSMYASLGERVTITCKASQDINSYLSWFQQKAGKSPKTLIYRANRLVDGVPSRFSGSGSGQDYSLTISSLEYEDMGIYYCLQYDEFPFTFGGGTRLEIK

SEQ ID NO: 465 - protein, 11 aa, synthetic construct
KASQDINSYLS

SEQ ID NO: 466 - protein, 7 aa, synthetic construct
RANRLVD

SEQ ID NO: 467 - protein, 9 aa, synthetic construct
LQYDEFPFT

SEQ ID NO: 468-1000 - skipped (533 entries)

SEQ ID NO: 1001 - protein, 119 aa, synthetic construct; identical to SEQ ID NO: 1

SEQ ID NO: 1002 - protein, 107 aa, synthetic construct; identical to SEQ ID NO: 2

SEQ ID NO: 1003 - protein, 10 aa, synthetic construct
GYSFTTYYIH

SEQ ID NO: 1004 - protein, 17 aa, synthetic construct
WFFPGSGNIKYNEKFKG

SEQ ID NO: 1005 - protein, 10 aa, synthetic construct
SYYSYDVLDY

SEQ ID NO: 1006 - protein, 11 aa, synthetic construct
KASQNVGINVV

SEQ ID NO: 1007 - protein, 7 aa, synthetic construct
SSSHRYS

SEQ ID NO: 1008 - protein, 9 aa, synthetic construct
QQFKSYPLT

SEQ ID NO: 1009 - protein, 119 aa, synthetic construct; identical to SEQ ID NO: 9

SEQ ID NO: 1010 - protein, 107 aa, synthetic construct; identical to SEQ ID NO: 10

SEQ ID NO: 1011 - protein, 107 aa, synthetic construct; identical to SEQ ID NO: 11

SEQ ID NO: 1012 - DNA, 357 bp, synthetic construct; identical to SEQ ID NO: 12
  source 1..357: mol_type = other DNA

SEQ ID NO: 1013 - DNA, 321 bp, synthetic construct; identical to SEQ ID NO: 13
  source 1..321: mol_type = other DNA

SEQ ID NO: 1014 - DNA, 321 bp, synthetic construct; identical to SEQ ID NO: 14
  source 1..321: mol_type = other DNA

SEQ ID NO: 1015 - protein, 7 aa, synthetic construct
GYSFTTY

SEQ ID NO: 1016 - protein, 6 aa, synthetic construct
FPGSGN

SEQ ID NO: 1017 - protein, 592 aa, synthetic construct; identical to SEQ ID NO: 17

SEQ ID NO: 1018 - protein, 492 aa, synthetic construct; identical to SEQ ID NO: 18

SEQ ID NO: 1019 - protein, 479 aa, synthetic construct; identical to SEQ ID NO: 19

SEQ ID NO: 1020 - protein, 469 aa, synthetic construct; identical to SEQ ID NO: 20

SEQ ID NO: 1021 - protein, 234 aa, synthetic construct; identical to SEQ ID NO: 21

SEQ ID NO: 1022 - protein, 246 aa, synthetic construct; identical to SEQ ID NO: 22

SEQ ID NO: 1023 - protein, 234 aa, synthetic construct; identical to SEQ ID NO: 23

SEQ ID NO: 1024 - protein, 107 aa, synthetic construct; identical to SEQ ID NO: 24

SEQ ID NO: 1025 - protein, 119 aa, synthetic construct; identical to SEQ ID NO: 25

SEQ ID NO: 1026 - protein, 246 aa, synthetic construct; identical to SEQ ID NO: 26

SEQ ID NO: 1027 - protein, 119 aa, synthetic construct; identical to SEQ ID NO: 27

SEQ ID NO: 1028 - protein, 246 aa, synthetic construct; identical to SEQ ID NO: 28

SEQ ID NO: 1029 - protein, 107 aa, synthetic construct; identical to SEQ ID NO: 29

SEQ ID NO: 1030 - protein, 119 aa, synthetic construct; identical to SEQ ID NO: 30

SEQ ID NO: 1031 - protein, 246 aa, synthetic construct; identical to SEQ ID NO: 31

SEQ ID NO: 1032 - protein, 107 aa, synthetic construct; identical to SEQ ID NO: 32

SEQ ID NO: 1033 - protein, 119 aa, synthetic construct; identical to SEQ ID NO: 33

SEQ ID NO: 1034 - protein, 246 aa, synthetic construct; identical to SEQ ID NO: 34

SEQ ID NO: 1035 - protein, 107 aa, synthetic construct; identical to SEQ ID NO: 35

SEQ ID NO: 1036 - protein, 119 aa, synthetic construct; identical to SEQ ID NO: 36

SEQ ID NO: 1037 - protein, 246 aa, synthetic construct; identical to SEQ ID NO: 37

SEQ ID NO: 1038 - protein, 119 aa, synthetic construct; identical to SEQ ID NO: 38

SEQ ID NO: 1039 - protein, 107 aa, synthetic construct [sequence omitted]

SEQ ID NO: 1040 - protein, 326 aa, synthetic construct; identical to SEQ ID NO: 40

SEQ ID NO: 1041 - protein, 330 aa, synthetic construct; identical to SEQ ID NO: 41

SEQ ID NO: 1042 - protein, 330 aa, synthetic construct; identical to SEQ ID NO: 42

SEQ ID NO: 1043 - protein, 344 aa, Homo sapiens
ATGAGCATCGGCCTCCTGTGCTGTGCAGCCTTGTCTCTCCTGTGGGCAGGTCCAGTGAATGCTGGTGTCACTCAGACCCCAAAATTCCAGGTCCTGAAGACAGGACAGAGCATGACACTGCAGTGTGCCCAGGATATGAACCATGAATACATGTCCTGGTATCGACAAGACCCAGGCATGGGGCTGAGGCTGATTCATTACTCAGTTGGTGCTGGTATCACTGACCAAGGAGAAGTCCCCAATGGCTACAATGTCTCCAGATCAACCACAGAGGATTTCCCGCTCAGGCTGCTGTCGGCTGCTCCCTCCCAGACATCTGTGTACTTCTGTGCCAGCAGTTACTC

SEQ ID NO: 1044 - protein, 114 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSY

SEQ ID NO: 1045 - protein, 5 aa, synthetic construct
TYYIH

SEQ ID NO: 1046 - protein, 246 aa, synthetic construct; identical to SEQ ID NO: 46

SEQ ID NO: 1047 - protein, 119 aa, synthetic construct; identical to SEQ ID NO: 47

SEQ ID NO: 1048 - protein, 246 aa, synthetic construct; identical to SEQ ID NO: 48

SEQ ID NO: 1049 - protein, 107 aa, synthetic construct; identical to SEQ ID NO: 49

SEQ ID NO: 1050 - protein, 119 aa, synthetic construct; identical to SEQ ID NO: 50

SEQ ID NO: 1051 - protein, 247 aa, synthetic construct; identical to SEQ ID NO: 51

SEQ ID NO: 1052 - protein, 107 aa, synthetic construct; identical to SEQ ID NO: 52

SEQ ID NO: 1053 - protein, 119 aa, synthetic construct; identical to SEQ ID NO: 53

SEQ ID NO: 1054 - protein, 246 aa, synthetic construct; identical to SEQ ID NO: 54

SEQ ID NO: 1055 - protein, 107 aa, synthetic construct; identical to SEQ ID NO: 52

SEQ ID NO: 1056 - protein, 119 aa, synthetic construct; identical to SEQ ID NO: 56

SEQ ID NO: 1057 - protein, 246 aa, synthetic construct; identical to SEQ ID NO: 57

SEQ ID NO: 1058 - protein, 107 aa, synthetic construct; identical to SEQ ID NO: 58

SEQ ID NO: 1059 - protein, 119 aa, synthetic construct; identical to SEQ ID NO: 59

SEQ ID NO: 1060 - protein, 246 aa, synthetic construct; identical to SEQ ID NO: 60

SEQ ID NO: 1061 - protein, 246 aa, synthetic construct; identical to SEQ ID NO: 61

SEQ ID NO: 1062 - protein, 107 aa, synthetic construct; identical to SEQ ID NO: 62

SEQ ID NO: 1063 - protein, 119 aa, synthetic construct; identical to SEQ ID NO: 63

SEQ ID NO: 1064 - protein, 246 aa, synthetic construct; identical to SEQ ID NO: 64

SEQ ID NO: 1065 - protein, 119 aa, synthetic construct; identical to SEQ ID NO: 65

SEQ ID NO: 1066 - protein, 246 aa, synthetic construct; identical to SEQ ID NO: 66

SEQ ID NO: 1067 - protein, 119 aa, synthetic construct; identical to SEQ ID NO: 67

SEQ ID NO: 1068 - protein, 246 aa, synthetic construct; identical to SEQ ID NO: 68

SEQ ID NO: 1069 - protein, 246 aa, synthetic construct; identical to SEQ ID NO: 69

SEQ ID NO: 1070 - protein, 119 aa, synthetic construct; identical to SEQ ID NO: 70

SEQ ID NO: 1071 - protein, 246 aa, synthetic construct; identical to SEQ ID NO: 71

SEQ ID NO: 1072 - protein, 119 aa, synthetic construct; identical to SEQ ID NO: 72

SEQ ID NO: 1073 - protein, 453 aa, synthetic construct; identical to SEQ ID NO: 73

SEQ ID NO: 1074 - protein, 353 aa, synthetic construct; identical to SEQ ID NO: 74

SEQ ID NO: 1075 - protein, 340 aa, synthetic construct; identical to SEQ ID NO: 75

SEQ ID NO: 1076 - protein, 159 aa, synthetic construct; identical to SEQ ID NO: 76

SEQ ID NO: 1077 - protein, 246 aa, synthetic construct; identical to SEQ ID NO: 77

SEQ ID NO: 1078 - protein, 119 aa, synthetic construct; identical to SEQ ID NO: 78

SEQ ID NO: 1079 - protein, 246 aa, synthetic construct; identical to SEQ ID NO: 79

SEQ ID NO: 1080 - protein, 119 aa, synthetic construct; identical to SEQ ID NO: 80

SEQ ID NO: 1081 - protein, 246 aa, synthetic construct; identical to SEQ ID NO: 81

SEQ ID NO: 1082 - protein, 107 aa, synthetic construct; identical to SEQ ID NO: 82

SEQ ID NO: 1083 - protein, 119 aa, synthetic construct; identical to SEQ ID NO: 83

SEQ ID NO: 1084 - protein, 246 aa, synthetic construct; identical to SEQ ID NO: 84

SEQ ID NO: 1085 - protein, 119 aa, synthetic construct; identical to SEQ ID NO: 85

SEQ ID NO: 1086 - protein, 246 aa, synthetic construct; identical to SEQ ID NO: 86

SEQ ID NO: 1087 - protein, 107 aa, synthetic construct; identical to SEQ ID NO: 87

SEQ ID NO: 1088 - protein, 119 aa, synthetic construct; identical to SEQ ID NO: 88

SEQ ID NO: 1089 - protein, 246 aa, synthetic construct; identical to SEQ ID NO: 89

SEQ ID NO: 1090 - protein, 119 aa, synthetic construct; identical to SEQ ID NO: 90

SEQ ID NO: 1091 - protein, 246 aa, synthetic construct; identical to SEQ ID NO: 91

SEQ ID NO: 1092 - protein, 119 aa, synthetic construct; identical to SEQ ID NO: 92

SEQ ID NO: 1093 - protein, 246 aa, synthetic construct; identical to SEQ ID NO: 93

SEQ ID NO: 1094 - protein, 119 aa, synthetic construct; identical to SEQ ID NO: 94

SEQ ID NO: 1095 - protein, 246 aa, synthetic construct; identical to SEQ ID NO: 95

SEQ ID NO: 1096 - protein, 107 aa, synthetic construct; identical to SEQ ID NO: 96

SEQ ID NO: 1097 - protein, 119 aa, synthetic construct; identical to SEQ ID NO: 97

SEQ ID NO: 1098 - protein, 246 aa, synthetic construct; identical to SEQ ID NO: 98

SEQ ID NO: 1099 - protein, 246 aa, synthetic construct; identical to SEQ ID NO: 99

SEQ ID NO: 1100 - protein, 119 aa, synthetic construct; identical to SEQ ID NO: 100

SEQ ID NO: 1101 - protein, 246 aa, synthetic construct; identical to SEQ ID NO: 101

SEQ ID NO: 1102 - protein, 107 aa, synthetic construct; identical to SEQ ID NO: 82

SEQ ID NO: 1103 - protein, 246 aa, synthetic construct; identical to SEQ ID NO: 103

SEQ ID NO: 1104 - protein, 119 aa, synthetic construct; identical to SEQ ID NO: 104

SEQ ID NO: 1105 - protein, 246 aa, synthetic construct; identical to SEQ ID NO: 105

SEQ ID NO: 1106 - protein, 119 aa, synthetic construct; identical to SEQ ID NO: 106

SEQ ID NO: 1107 - protein, 246 aa, synthetic construct; identical to SEQ ID NO: 107

SEQ ID NO: 1108 - protein, 119 aa, synthetic construct; identical to SEQ ID NO: 108

SEQ ID NO: 1109 - protein, 246 aa, synthetic construct; identical to SEQ ID NO: 109

SEQ ID NO: 1110 - protein, 107 aa, synthetic construct; identical to SEQ ID NO: 110

SEQ ID NO: 1111 - protein, 119 aa, synthetic construct; identical to SEQ ID NO: 111

SEQ ID NO: 1112 - protein, 246 aa, synthetic construct; identical to SEQ ID NO: 112

SEQ ID NO: 1113 - protein, 119 aa, synthetic construct; identical to SEQ ID NO: 113

SEQ ID NO: 1114 - protein, 246 aa, synthetic construct; identical to SEQ ID NO: 114

SEQ ID NO: 1115 - protein, 119 aa, synthetic construct; identical to SEQ ID NO: 115

SEQ ID NO: 1116 - protein, 246 aa, synthetic construct; identical to SEQ ID NO: 116

SEQ ID NO: 1117 - protein, 119 aa, synthetic construct; identical to SEQ ID NO: 117

SEQ ID NO: 1118 - protein, 246 aa, synthetic construct; identical to SEQ ID NO: 118

SEQ ID NO: 1119 - protein, 246 aa, synthetic construct; identical to SEQ ID NO: 119

SEQ ID NO: 1120 - protein, 119 aa, synthetic construct; identical to SEQ ID NO: 120

SEQ ID NO: 1121 - protein, 246 aa, synthetic construct; identical to SEQ ID NO: 121

SEQ ID NO: 1122 - protein, 107 aa, synthetic construct; identical to SEQ ID NO: 122

SEQ ID NO: 1123 - protein, 119 aa, synthetic construct; identical to SEQ ID NO: 123

SEQ ID NO: 1124 - protein, 246 aa, synthetic construct; identical to SEQ ID NO: 124

SEQ ID NO: 1125 - protein, 119 aa, synthetic construct; identical to SEQ ID NO: 125

SEQ ID NO: 1126 - protein, 246 aa, synthetic construct; identical to SEQ ID NO: 126

SEQ ID NO: 1127 - protein, 119 aa, synthetic construct; identical to SEQ ID NO: 127

SEQ ID NO: 1128 - protein, 246 aa, synthetic construct; identical to SEQ ID NO: 128

SEQ ID NO: 1129 - protein, 119 aa, synthetic construct; identical to SEQ ID NO: 129

SEQ ID NO: 1130 - protein, 246 aa, synthetic construct; identical to SEQ ID NO: 130

SEQ ID NO: 1131 - protein, 119 aa, synthetic construct; identical to SEQ ID NO: 131

SEQ ID NO: 1132 - protein, 246 aa, synthetic construct; identical to SEQ ID NO: 132

SEQ ID NO: 1133 - protein, 119 aa, synthetic construct; identical to SEQ ID NO: 133

SEQ ID NO: 1134 - protein, 246 aa, synthetic construct; identical to SEQ ID NO: 134

SEQ ID NO: 1135 - protein, 119 aa, synthetic construct; identical to SEQ ID NO: 135

SEQ ID NO: 1136 - protein, 246 aa, synthetic construct; identical to SEQ ID NO: 136

SEQ ID NO: 1137 - protein, 119 aa, synthetic construct; identical to SEQ ID NO: 137

SEQ ID NO: 1138 - protein, 246 aa, synthetic construct; identical to SEQ ID NO: 138

SEQ ID NO: 1139 - protein, 119 aa, synthetic construct; identical to SEQ ID NO: 139

SEQ ID NO: 1140 - protein, 246 aa, synthetic construct; identical to SEQ ID NO: 140

SEQ ID NO: 1141 - protein, 119 aa, synthetic construct; identical to SEQ ID NO: 141

SEQ ID NO: 1142 - protein, 246 aa, synthetic construct; identical to SEQ ID NO: 142

SEQ ID NO: 1143 - protein, 119 aa, synthetic construct; identical to SEQ ID NO: 143

SEQ ID NO: 1144 - protein, 246 aa, synthetic construct; identical to SEQ ID NO: 144

SEQ ID NO: 1145 - protein, 119 aa, synthetic construct; identical to SEQ ID NO: 145

SEQ ID NO: 1146 - protein, 246 aa, synthetic construct; identical to SEQ ID NO: 146

SEQ ID NO: 1147 - protein, 119 aa, synthetic construct; identical to SEQ ID NO: 147

SEQ ID NO: 1148 - protein, 106 aa, synthetic construct; identical to SEQ ID NO: 148

SEQ ID NO: 1149 - protein, 246 aa, synthetic construct; identical to SEQ ID NO: 149

SEQ ID NO: 1150 - protein, 246 aa, synthetic construct; identical to SEQ ID NO: 150

SEQ ID NO: 1151 - protein, 246 aa, synthetic construct; identical to SEQ ID NO: 151

SEQ ID NO: 1152 - protein, 246 aa, synthetic construct; identical to SEQ ID NO: 152

SEQ ID NO: 1153 - protein, 246 aa, synthetic construct; identical to SEQ ID NO: 153

SEQ ID NO: 1154 - protein, 246 aa, synthetic construct; identical to SEQ ID NO: 154

SEQ ID NO: 1155 - protein, 246 aa, synthetic construct; identical to SEQ ID NO: 155

SEQ ID NO: 1156 - protein, 246 aa, synthetic construct; identical to SEQ ID NO: 156

SEQ ID NO: 1157 - protein, 246 aa, synthetic construct; identical to SEQ ID NO: 157

SEQ ID NO: 1158 - protein, 246 aa, synthetic construct; identical to SEQ ID NO: 158

SEQ ID NO: 1159 - protein, 246 aa, synthetic construct; identical to SEQ ID NO: 159

SEQ ID NO: 1160 - protein, 246 aa, synthetic construct; identical to SEQ ID NO: 160

SEQ ID NO: 1161 - protein, 246 aa, synthetic construct; identical to SEQ ID NO: 161

SEQ ID NO: 1162 - protein, 119 aa, synthetic construct; identical to SEQ ID NO: 162

SEQ ID NO: 1163 - protein, 119 aa, synthetic construct; identical to SEQ ID NO: 163

SEQ ID NO: 1164 - protein, 10 aa, synthetic construct
GHDFRLTYIH

SEQ ID NO: 1165 - protein, 17 aa, synthetic construct
RVSAGSGNVKYNEKFKG

SEQ ID NO: 1166 - protein, 11 aa, synthetic construct
KASQNVGNRVV

SEQ ID NO: 1167 - protein, 7 aa, synthetic construct
SSSHRYK

SEQ ID NO: 1168 - protein, 119 aa, synthetic construct; identical to SEQ ID NO: 168

SEQ ID NO: 1169 - protein, 107 aa, synthetic construct; identical to SEQ ID NO: 169

SEQ ID NO: 1170 - protein, 17 aa, synthetic construct
RIYAGSGNVKYNEKFKG

SEQ ID NO: 1171 - protein, 11 aa, synthetic construct
KASQNVADRVV

SEQ ID NO: 1172 - protein, 119 aa, synthetic construct; identical to SEQ ID NO: 172

SEQ ID NO: 1173 - protein, 107 aa, synthetic construct; identical to SEQ ID NO: 173

SEQ ID NO: 1174 - protein, 10 aa, synthetic construct
GHDFKLTYIH

SEQ ID NO: 1175 - protein, 17 aa, synthetic construct
RVSAGSGNTKYNEKFKG

SEQ ID NO: 1176 - protein, 11 aa, synthetic construct
KASQNVDNRVA

SEQ ID NO: 1177 - protein, 119 aa, synthetic construct; identical to SEQ ID NO: 177

SEQ ID NO: 1178 - protein, 17 aa, synthetic construct
RVYAGSGNTKYNEKFKG

SEQ ID NO: 1179 - protein, 11 aa, synthetic construct
KASQNVEDRVV

SEQ ID NO: 1180 - protein, 119 aa, synthetic construct; identical to SEQ ID NO: 180

SEQ ID NO: 1181 - protein, 17 aa, synthetic construct
RISAGSGNTKYNEKFKG

SEQ ID NO: 1182 - protein, 11 aa, synthetic construct
KASQNVGDRVV

SEQ ID NO: 1183 - protein, 119 aa, synthetic construct; identical to SEQ ID NO: 183

SEQ ID NO: 1184 - protein, 107 aa, synthetic construct; identical to SEQ ID NO: 184

SEQ ID NO: 1185 - protein, 119 aa, synthetic construct; identical to SEQ ID NO: 185

SEQ ID NO: 1186 - protein, 11 aa, synthetic construct
RASQNVDNRLG

SEQ ID NO: 1187 - protein, 107 aa, synthetic construct; identical to SEQ ID NO: 187

SEQ ID NO: 1188 - protein, 7 aa, synthetic construct
AASSLQK

SEQ ID NO: 1189 - protein, 107 aa, synthetic construct; identical to SEQ ID NO: 189

SEQ ID NO: 1190 - protein, 9 aa, synthetic construct
LQHNSYPLT

SEQ ID NO: 1191 - protein, 107 aa, synthetic construct; identical to SEQ ID NO: 191

SEQ ID NO: 1192 - protein, 17 aa, synthetic construct
RVSAGSGNVNYAQKFQG

SEQ ID NO: 1193 - protein, 119 aa, synthetic construct; identical to SEQ ID NO: 193

SEQ ID NO: 1194 - protein, 119 aa, synthetic construct; identical to SEQ ID NO: 194

SEQ ID NO: 1195 - protein, 113 aa, synthetic construct; identical to SEQ ID NO: 195

SEQ ID NO: 1196 - protein, 98 aa, synthetic construct; identical to SEQ ID NO: 196

SEQ ID NO: 1197 - protein, 8 aa, synthetic construct
GFNIKDTH

SEQ ID NO: 1198 - protein, 8 aa, synthetic construct
TDPASGDT

SEQ ID NO: 1199 - protein, 15 aa, synthetic construct
CAHYYRDDVNYAMDY

SEQ ID NO: 1200 - protein, 6 aa, synthetic construct
QNVGSN

SEQ ID NO: 1201 - skipped

SEQ ID NO: 1202 - protein, 9 aa, synthetic construct
QQYNTYPYT

SEQ ID NO: 1203 - protein, 107 aa, synthetic construct [sequence omitted]

SEQ ID NO: 1204 - protein, 330 aa, synthetic construct; identical to SEQ ID NO: 204

SEQ ID NO: 1205 - protein, 334 aa, synthetic construct; identical to SEQ ID NO: 205

SEQ ID NO: 1206 - protein, 227 aa, synthetic construct; identical to SEQ ID NO: 206

SEQ ID NO: 1207 - protein, 330 aa, synthetic construct; identical to SEQ ID NO: 207

SEQ ID NO: 1208 - protein, 5 aa, synthetic construct
DYYMY

SEQ ID NO: 1209 - protein, 17 aa, synthetic construct
TISGGGSYTYSPDSVKG

SEQ ID NO: 1210 - protein, 14 aa, synthetic construct
ERDIYYGNFNAMVY

SEQ ID NO: 1211 - protein, 7 aa, synthetic construct
GFTFSDY

SEQ ID NO: 1212 - protein, 6 aa, synthetic construct
SGGGSY

SEQ ID NO: 1213 - protein, 10 aa, synthetic construct
GFTFSDYYMY

SEQ ID NO: 1214 - protein, 15 aa, synthetic construct
RASKSVSTSGYSYMH

SEQ ID NO: 1215 - protein, 7 aa, synthetic construct
LASNLES

SEQ ID NO: 1216 - protein, 9 aa, synthetic construct
QHSRDLPWT

SEQ ID NO: 1217 - protein, 11 aa, synthetic construct
SKSVSTSGYSY

SEQ ID NO: 1218 - protein, 111 aa, synthetic construct
DIVLTQSPVSLTVSLGQRATISCRASKSVSTSGYSYMHWYQQKPGQPPKLLIYLASNLESGVPARFSGSGSGTDFTLNIHPVEEEDAATYYCQHSRDLPWTFGGGTKLEIK

SEQ ID NO: 1219 - protein, 123 aa, synthetic construct
EVQLVESGGGLVKPGGSLKLSCAASGFTFSDYYMYWVRQTPEKRLEWVATISGGGSYTYSPDSVKGRFTISRDNAKNNLYLQMSSLRSEDTAMYFCARERDIYYGNFNAMVYWGRGTSVTVSS

SEQ ID NO: 1220 - protein, 123 aa, synthetic construct
EVQLLESGGGLVQPGGSLRLSCAASGFTFSDYYMYWVRQAPGKGLEWVATISGGGSYTYSPDSVKGRFTISRDNSKNTLYLQMNSLRAEDTAVYYCARERDIYYGNFNAMVYWGRGTLVTVSS

SEQ ID NO: 1221 - protein, 123 aa, synthetic construct
EVQLVESGGGLVQPGGSLRLSCAASGFTFSDYYMYWVRQAPGKGLEWVATISGGGSYTYSPDSVKGRFTISRDNAKNSLYLQMNSLRAEDTAVYYCARERDIYYGNFNAMVYWGRGTLVTVSS

SEQ ID NO: 1222 - protein, 123 aa, synthetic construct
QVQLVESGGGVVQPGRSLRLSCAASGFTFSDYYMYWVRQAPGKGLEWVATISGGGSYTYSPDSVKGRFTISRDNSKNTLYLQMNSLRAEDTAVYYCARERDIYYGNFNAMVYWGRGTLVTVSS

SEQ ID NO: 1223 - protein, 123 aa, synthetic construct
QVQLVESGGGLVKPGGSLRLSCAASGFTFSDYYMYWIRQAPGKGLEWVATISGGGSYTYSPDSVKGRFTISRDNAKNSLYLQMNSLRAEDTAVYYCARERDIYYGNFNAMVYWGRGTLVTVSS

SEQ ID NO: 1224 - protein, 111 aa, synthetic construct
EIVLTQSPGTLSLSPGERATLSCRASKSVSTSGYSYMHWYQQKPGQAPRLLIYLASNLESGIPDRFSGSGSGTDFTLTISRLEPEDFAVYYCQHSRDLPWTFGGGTKVEIK

SEQ ID NO: 1225 - protein, 111 aa, synthetic construct
EIVLTQSPATLSLSPGERATLSCRASKSVSTSGYSYMHWYQQKPGQAPRLLIYLASNLESGIPARFSGSGSGTDFTLTISSLEPEDFAVYYCQHSRDLPWTFGGGTKVEIK

SEQ ID NO: 1226 - protein, 111 aa, synthetic construct
DIQLTQSPSTLSASVGDRVTITCRASKSVSTSGYSYMHWYQQKPGKAPKLLIYLASNLESGVPSRFSGSGSGTEFTLTISSLQPDDFATYYCQHSRDLPWTFGGGTKVEIK

SEQ ID NO: 1227 - protein, 111 aa, synthetic construct
AIQLTQSPSSLSASVGDRVTITCRASKSVSTSGYSYMHWYQQKPGKAPKLLIYLASNLESGVPSRFSGSGSGTDFTLTISSLQPEDFATYYCQHSRDLPWTFGGGTKVEIK

SEQ ID NO: 1228 - protein, 449 aa, synthetic construct; identical to SEQ ID NO: 228

SEQ ID NO: 1229 - protein, 5 aa, synthetic construct
LTYIH

SEQ ID NO: 1230 - protein, 107 aa, synthetic construct
  VARIANT 30: note = G, E, A or D
  VARIANT 31: note = N or D
  VARIANT 32: note = R or K
  VARIANT 36: note = Y or H
  VARIANT 56: note = K or S
DIQMTQSPSFLSASVGDRVTITCKASQNVXXXVAWXQQKPGKAPKALIYSSSHRYXGVPSRFSGSGSGTEFTLTISSLQPEDFATYFCQQFKSYPLTFGQGTKLEIK

SEQ ID NO: 1231 - protein, 123 aa, synthetic construct
  VARIANT 27: note = H, T, G or Y
  VARIANT 28: note = D, T or S
  VARIANT 30: note = H, R, D, K or T
  VARIANT 31: note = L, D, K, T or N
  VARIANT 32: note = W, F, T, I, Y or G
  VARIANT 50: note = R or W
  VARIANT 51: note = V, I or F
  VARIANT 52: note = F, S or Y
  VARIANT 53: note = A or P
  VARIANT 57: note = N or S
  VARIANT 58: note = T, V, Y or I
  VARIANT 59: note = K or R
  VARIANT 98: note = G or V
  VARIANT 100: note = Y or I
  VARIANT 103: note = Y or A
  VARIANT 104: note = D or G
QVQLVQSGAEVKKPGSSVKVSCKASGXXFXXXYIHWVRQAPGQGLEWMGXXXXGSGXXXYNEKFKGRVTITADTSTSTAYMELSSLRSEDTAVYYCAGVSYIYSYADGVLDYWGQGTTVTVSS

SEQ ID NO: 1232 - protein, 107 aa, synthetic construct; identical to SEQ ID NO: 1230
  VARIANT 30: note = G, E, A or D
  VARIANT 32: note = N or D
  VARIANT 34: note = R or K
  VARIANT 39: note = Y or H
  VARIANT 60: note = K or S

SEQ ID NO: 1233 - protein, 119 aa, synthetic construct
  VARIANT 27: note = H, T, G or Y
  VARIANT 28: note = D, T or S
  VARIANT 30: note = H, R, D, K or T
  VARIANT 31: note = L, D, K, T or N
  VARIANT 32: note = W, F, T, I, Y or G
  VARIANT 50: note = R or W
  VARIANT 51: note = V, I or F
  VARIANT 52: note = F, S, or Y
  VARIANT 53: note = A or P
  VARIANT 57: note = N or S
  VARIANT 58: note = T, V, Y or I
  VARIANT 59: note = K or R
  VARIANT 98: note = G or V
  VARIANT 100: note = Y or I
  VARIANT 103: note = Y or A
  VARIANT 104: note = D or G
QVQLVQSGAEVKKPGSSVKVSCKASGXXFXXXYIHWVRQAPGQGLEWMGXXXXGSGXXXYNEKFKGRVTITADTSTSTAYMELSSLRSEDTAVYYCAXSXYSXXVLDYWGQGTTVTVSS

SEQ ID NO: 1234 - protein, 7 aa, synthetic construct
FRLTYIH

SEQ ID NO: 1235 - protein, 448 aa, synthetic construct; identical to SEQ ID NO: 235

SEQ ID NO: 1236 - protein, 77 aa, synthetic construct; identical to SEQ ID NO: 236

SEQ ID NO: 1237 - protein, 10 aa, synthetic construct
GGGGSGGGGS

SEQ ID NO: 1238 - protein, 114 aa, Homo sapiens; identical to SEQ ID NO: 238

SEQ ID NO: 1239 - protein, 214 aa, synthetic construct; identical to SEQ ID NO: 239

SEQ ID NO: 1240 - protein, 351 aa, synthetic construct; identical to SEQ ID NO: 240

SEQ ID NO: 1241 - protein, 370 aa, synthetic construct; identical to SEQ ID NO: 241

SEQ ID NO: 1242 - protein, 133 aa, synthetic construct; identical to SEQ ID NO: 242

SEQ ID NO: 1243 - protein, 20 aa, synthetic construct
SGGSGGGGSGGGSGGGGSGG

SEQ ID NO: 1244 - protein, 452 aa, synthetic construct; identical to SEQ ID NO: 244

SEQ ID NO: 1245 - protein, 448 aa, synthetic construct; identical to SEQ ID NO: 245

SEQ ID NO: 1246 - protein, 192 aa, synthetic construct; identical to SEQ ID NO: 246

SEQ ID NO: 1247 - protein, 10 aa, synthetic construct
GYTFSNYWIE

SEQ ID NO: 1248 - protein, 17 aa, synthetic construct
EILPGAGPTNYNEKFKG

SEQ ID NO: 1249 - protein, 10 aa, synthetic construct
TDYDYDWFAY

SEQ ID NO: 1250 - protein, 260 aa, synthetic construct; identical to SEQ ID NO: 250

SEQ ID NO: 1251 - protein, 368 aa, synthetic construct; identical to SEQ ID NO: 251

SEQ ID NO: 1252 - protein, 113 aa, synthetic construct; identical to SEQ ID NO: 252

SEQ ID NO: 1253 - protein, 11 aa, synthetic construct
KSSQSLLYSGN

SEQ ID NO: 1254 - protein, 8 aa, synthetic construct
PKLLIYWA

SEQ ID NO: 1255 - protein, 9 aa, synthetic construct
QQYYGYPRT

SEQ ID NO: 1256 - protein, 107 aa, synthetic construct; identical to SEQ ID NO: 256

SEQ ID NO: 1257 - protein, 452 aa, synthetic construct; identical to SEQ ID NO: 257

SEQ ID NO: 1258 - protein, 15 aa, synthetic construct
GGGGSGGGGSGGGGS

SEQ ID NO: 1259 - protein, 5 aa, synthetic construct
GGGGS

SEQ ID NO: 1260 - protein, 14 aa, synthetic construct
AEAAAKEAAAKAAA

SEQ ID NO: 1261 - protein, 19 aa, synthetic construct
AEAAAKEAAAKEAAAKAAA

SEQ ID NO: 1262 - protein, 24 aa, synthetic construct
AEAAAKEAAAKEAAAKEAAAKAAA

SEQ ID NO: 1263 - protein, 29 aa, synthetic construct
AEAAAKEAAAKEAAAKEAAAKEAAAKAAA

SEQ ID NO: 1264 - protein, 256 aa, synthetic construct; identical to SEQ ID NO: 264

SEQ ID NO: 1265 - protein, 4 aa, synthetic construct
EPEA

SEQ ID NO: 1266 - protein, 383 aa, synthetic construct; identical to SEQ ID NO: 266

SEQ ID NO: 1267 - protein, 370 aa, synthetic construct; identical to SEQ ID NO: 267

SEQ ID NO: 1268 - protein, 133 aa, Homo sapiens [sequence omitted]

SEQ ID NO: 1269 - protein, 227 aa, synthetic construct; identical to SEQ ID NO: 269

SEQ ID NO: 1270 - protein, 13 aa, synthetic construct
GGGGSHHHHHHHH

SEQ ID NO: 1271 - protein, 220 aa, synthetic construct; identical to SEQ ID NO: 271

SEQ ID NO: 1272 - protein, 389 aa, synthetic construct; identical to SEQ ID NO: 272

SEQ ID NO: 1273 - protein, 152 aa, synthetic construct; identical to SEQ ID NO: 273

SEQ ID NO: 1274 - protein, 133 aa, synthetic construct [sequence omitted]

SEQ ID NO: 1275 - protein, 755 aa, synthetic construct; identical to SEQ ID NO: 275

SEQ ID NO: 1276 - protein, 518 aa, synthetic construct; identical to SEQ ID NO: 276

SEQ ID NO: 1277 - protein, 305 aa, synthetic construct; identical to SEQ ID NO: 277

SEQ ID NO: 1278 - protein, 16 aa, synthetic construct
SGGGGSGGGGSGGGGS

SEQ ID NO: 1279 - protein, 197 aa, synthetic construct; identical to SEQ ID NO: 279

SEQ ID NO: 1280 - protein, 133 aa, Homo sapiens; identical to SEQ ID NO: 445

SEQ ID NO: 1281 - protein, 340 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 1282 - protein, 166 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 1283 - protein, 77 aa, synthetic construct
MAPRRARGCRTLGLPALLLLLLLRPPATRGITCPPPMSVEHADIWVKSYSLYSRERYICNSGFKRKAGTSSLTECVL

SEQ ID NO: 1284 - protein, 20 aa, synthetic construct
SGGSGGGGSGGGSGGGGSLQ

SEQ ID NO: 1285 - protein, 157 aa, Homo sapiens; identical to SEQ ID NO: 413

SEQ ID NO: 1286 - protein, 133 aa, Homo sapiens [sequence omitted]

SEQ ID NO: 1287 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 1288 - protein, 370 aa, synthetic construct; identical to SEQ ID NO: 288

SEQ ID NO: 1289 - protein, 447 aa, synthetic construct; identical to SEQ ID NO: 289

SEQ ID NO: 1290 - protein, 20 aa, synthetic construct
METDTLLLWVLLLWVPGSTG

SEQ ID NO: 1291 - protein, 272 aa, synthetic construct
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGLNDIFEAQKIEWHE

SEQ ID NO: 1292 - protein, 449 aa, synthetic construct; identical to SEQ ID NO: 292

SEQ ID NO: 1293 - protein, 328 aa, synthetic construct; identical to SEQ ID NO: 293

SEQ ID NO: 1294 - protein, 225 aa, synthetic construct; identical to SEQ ID NO: 294

SEQ ID NO: 1295 - protein, 78 aa, synthetic construct; identical to SEQ ID NO: 295

SEQ ID NO: 1296 - protein, 20 aa, synthetic construct
GGSGGGGSGGGSGGGGSGGG

SEQ ID NO: 1297 - protein, 212 aa, synthetic construct; identical to SEQ ID NO: 297

SEQ ID NO: 1298 - protein, 214 aa, synthetic construct; identical to SEQ ID NO: 298

SEQ ID NO: 1299 - protein, 7 aa, synthetic construct
AASYLQS

SEQ ID NO: 1300 - protein, 107 aa, synthetic construct; identical to SEQ ID NO: 300

SEQ ID NO: 1301 - protein, 214 aa, synthetic construct
DIQMTQSPSFLSASVGDRVTITCKASQNVADRVVWHQQKPGKAPKALIFAASYLQSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1302 - protein, 27 aa, synthetic construct
AEAAAKEAAAKEAAAKEAAAKEAAAKA

SEQ ID NO: 1303 - protein, 107 aa, synthetic construct; identical to SEQ ID NO: 303

SEQ ID NO: 1304 - protein, 449 aa, synthetic construct; identical to SEQ ID NO: 304

SEQ ID NO: 1305 - protein, 17 aa, synthetic construct
DVPSGPGGGGGSGGGGS

SEQ ID NO: 1306 - protein, 16 aa, synthetic construct
GGGGSGGGGSGGGGGS

SEQ ID NO: 1307 - protein, 114 aa, synthetic construct; identical to SEQ ID NO: 307

SEQ ID NO: 1308 - protein, 212 aa, synthetic construct; identical to SEQ ID NO: 308

SEQ ID NO: 1309 - protein, 449 aa, synthetic construct; identical to SEQ ID NO: 309

SEQ ID NO: 1310 - protein, 114 aa, synthetic construct; identical to SEQ ID NO: 310

SEQ ID NO: 1311 - protein, 212 aa, synthetic construct; identical to SEQ ID NO: 311

SEQ ID NO: 1312 - protein, 114 aa, synthetic construct; identical to SEQ ID NO: 411

SEQ ID NO: 1313 - protein, 11 aa, synthetic construct
KASQNVGDRVA

SEQ ID NO: 1314 - protein, 17 aa, synthetic construct
RIYAGSGNTKYNEKFKG

SEQ ID NO: 1315 - protein, 368 aa, synthetic construct; identical to SEQ ID NO: 352

SEQ ID NO: 1316 - protein, 225 aa, synthetic construct; identical to SEQ ID NO: 353

SEQ ID NO: 1317 - protein, 107 aa, synthetic construct
DIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 1318 - protein, 10 aa, synthetic construct
FPAVLQSDLY

(19) United States
(12) Patent Application Publication
(10) Pub. No.: US 2026/0158162 A1
(43) Pub. Date: Jun. 11, 2026

(54) MULTISPECIFIC MOLECULES BINDING TO TCR AND USES THEREOF

(71) Applicant: Marengo Therapeutics, Inc., Cambridge, MA (US)

(72) Inventor: Andrew Bayliffe, London (GB)

(21) Appl. No.: 19/408,507

(22) Filed: Dec. 4, 2025

Related U.S. Application Data
(63) Continuation of application No. PCT/US24/33300, filed on Jun. 10, 2024.
(60) Provisional application No. 63/507,153, filed on Jun. 9, 2023.
(60) Provisional application No. 63/507,155, filed on Jun. 9, 2023.
(60) Provisional application No. 63/507,156, filed on Jun. 9, 2023.

(51) Int. Cl. A61K 47/68; C07K 16/28

(52) U.S. Cl. CPC A61K 47/6849; A61K 47/6813; C07K 16/2809

(57) ABSTRACT

Provides herein are multifunctional polypeptide molecules comprising T cell receptor variable alpha-binding moieties, T cell receptor variable beta-binding moieties, TAA-binding moieties, and/or cytokines, and methods of treating conditions or diseases in a subject using the same.

### CROSS REFERENCE TO RELATED APPLICATIONS

This application is continuation of International Application No. PCT/US24/33300 filed on Jun. 10, 2024, which claims priority to U.S. Ser. No. 63/507,153 filed on Jun. 9, 2023, and U.S. Ser. No. 63/507,155 filed on Jun. 9, 2023, and U.S. Ser. No. 63/507,156 filed on Jun. 9, 2023, the contents of which are incorporated herein by reference in their entireties.

### REFERENCE TO A SEQUENCE LISTING

The instant application contains a Sequence Listing which has been submitted electronically in XML format and is hereby incorporated by reference in its entirety. Said XML copy, created on Dec. 2, 2025, is named 53676-760.301_SL.xml and is 846,856 bytes in size.

### BACKGROUND

Mucosal-associated invariant T (MAIT) cells are a newly described subset of T cells that are found in the blood, liver, lungs, and mucosa, defending against microbial activity and infection. In total, MAIT cells make up roughly 5% of the peripheral T cell population. MAIT cells are most common in the liver, where they usually comprise 20-40% of the T lymphocyte population. MAIT cells, contrast with conventional T cells which have highly variable TCRs, express a semi-invariant T cell receptor restricted by the MHC class I-related (MR1) molecule, a β2-microglobulin-associated antigen-presenting molecule, widely expressed in multiple tissues. After the presentation of foreign antigen by MR1, MAIT cells secrete pro-inflammatory cytokines and are capable of lysing bacterially-infected cells. In the absence of TCR-mediated antigen recognition, MAIT cells can also be activated by cytokines, broadening the potential range of pathogens to which MAIT cells can respond to include viruses. TCR-mediated and -independent activation work synergistically in optimal MAIT cell activation.

Invariant natural killer T (iNKT) cells, also known as type I or classical NKT cells, are a distinct population of T cells that express an invariant aI3 T-cell receptor (TCR) and a number of cell surface molecules in common with natural killer (NK) cells, iNKT cells are known for their ability to respond rapidly to danger signals and pro-inflammatory cytokines. Once activated, iNKT cells engage in effector functions, e.g., NK transactivation, T cell activation and differentiation, B cell activation, dendritic cell activation and cross-presentation activity, and macrophage activation. A decline in the number of iNKTs is often accompanied by their functional alternation in patients with many types of tumors. The prototypic antigen for NKT cells is a marine sponge-derived glycolipid, a-galactosylceramide (a-GalCer), a-GalCer has been used in clinical trial for treating cancer, but met little success and did not show any clinical improvement. &e, e.g., Giaccone G, et al., Clin Cancer Res. 8:3702-3709 (2002). Thus, there is need for alternative modalities to activate and/or expand iNKT cells, or to treat diseases such as cancer that involve depletion, anergy, and/or dysfunction of iNKT cells.

Currently available molecules designed to redirect T cells to promote tumor cell lysis for cancer immunotherapy typically target the CD3 epsilon (CD3e) subunit of the T cell receptor (TCR). However, there are limitations to this approach, anti-CD3e mAbs bind to all T cells and thus activate a large number of T cells. Such non-physiological massive activation of T cells by these anti-CD3e mAbs can result in the production of proinflammatory cytokines such as IFN-gamma, IL-1-beta, IL-6, IL-10 and TNF-alpha, causing a “cytokine storm” known as the cytokine release syndrome (CRS), which is also associated with neurotoxicity (NT). Certain tumor cells express Cd1b or Cd1c antigen-presenting molecules, which are recognized by T cell clones specific for the CD1b or Cd1c antigen-presenting molecules. Thus, there is a need for improved T cell receptor-binding molecules that activate and redirect a specific subset of T cells for cancer immunotherapy.

### SUMMARY

### Part I

In one aspect, provided herein is a composition comprising a multispecific molecule, wherein the multispecific molecule comprises: (a) a first domain that binds to a first target molecule, wherein the first target molecule is T cell receptor alpha variable 1-2 (TRAV1-2), T cell receptor beta variable 20 (TRBV20), T cell receptor beta variable 6 (TRBV6), or both TRBV20 and TRBV6, and (b) a second domain that binds to a second target molecule, wherein the second domain comprises (i) a cytokine or a functional fragment or variant thereof, or (ii) an antigen binding domain. In some embodiments, the composition further comprises a mucosal-associated invariant T (MAIT) cell. In some embodiments, the multispecific molecule is bound to the MAIT cell.

In some embodiments, the multispecific molecule is an agonist of TRAV1-2, TRBV20, TRBV6, or both TRBV20 and TRBV6. In some embodiments, the multispecific molecule is an antagonist of TRAV1-2, TRBV20, TRBV6, or both TRBV20 and TRBV6. In some embodiments, the first domain binds to TRAV1-2. In some embodiments, the first domain binds to TRBV20. In some embodiments. TRBV20 comprises TRBV20-1. In some embodiments, TRBV20-1 comprises TRBV20-1*01, TRBV20-1*02, TRBV20-1*03, TRBV20-1*04, TRBV20-1*05, TRBV20-1*06, or TRBV20-1*07. In some embodiments, the first domain binds to TRBV6. In some embodiments, TRBV6 comprises TRBV6-1, TRBV6-2, TRBV6-3, TRBV6-4, TRBV6-5, TRBV6-6, TRBV6-8, or TRBV6-9. In some embodiments. TRBV6-1 comprises TRBV6-1*01. In some embodiments, TRBV6-2 comprises TRBV6-2*01. In some embodiments, TRBV6-3 comprises TRBV6-3*01. In some embodiments, TRBV6-4 comprises TRBV6-4*01, or TRBV6-4*02. In some embodiments, TRBV6-5 comprises TRBV6-5*01. In some embodiments, TRBV6-6 comprises TRBV6-6*01, TRBV6-6*02, TRBV6-6*03, TRBV6-6*04, or TRBV6-6*05. In some embodiments, TRBV6-8 comprises TRBV6-8*01. In some embodiments, TRBV6-9 comprises TRBV6-9*01. In some embodiments, the first domain binds to both TRBV6 and TRBV20.

In some embodiments, the second domain comprises a cytokine. In some embodiments, the cytokine is any one selected from the group consisting of an interleukin-2 (IL-2) molecule or functional fragment or variant thereof, an interleukin-7 (IL-7) molecule or functional fragment or variant thereof, an interleukin-12 (IL-12) molecule or functional fragment or variant thereof, an interleukin-15 (IL-15) molecule or functional fragment or variant thereof, an interleukin-18 (IL-18) molecule or functional fragment or variant thereof, an interleukin-21 (IL-21) molecule or functional fragment or variant thereof, an interferon gamma molecule or functional fragment or variant thereof, and any combination thereof. In some embodiments, the cytokine comprises IL-2 or a functional fragment or variant thereof. In some embodiments, the IL-2 or the functional fragment or variant thereof comprises a sequence having at least 75% sequence identity to the sequence of SEQ ID NO: 268. In some embodiments, the cytokine comprises IL-7 or a functional fragment or variant thereof. In some embodiments, the IL-7 or the functional fragment or variant thereof comprises a sequence having at least 75% sequence identity to the sequence of SEQ ID NO: 273. In some embodiments, the cytokine comprises IL-12 or a functional fragment or variant thereof. In some embodiments, the IL-12 or the functional fragment or variant thereof comprises a sequence having at least 75% sequence identity to the sequence of SEQ ID NO: 276. In some embodiments, the cytokine comprises IL-15 or a functional fragment or variant thereof. In some embodiments, the IL-15 or the functional fragment or variant thereof comprises a sequence having at least 75% sequence identity to the sequence of SEQ ID NO: 297. In some embodiments, the cytokine comprises IL-18 or a functional fragment or variant thereof. In some embodiments, the IL-18 or the functional fragment or variant thereof comprises a sequence having at least 75% sequence identity to the sequence of SEQ ID NO: 285. In some embodiments, the cytokine comprises IL-21 or a functional fragment or variant thereof. In some embodiments, the IL-21 or the functional fragment or variant thereof comprises a sequence having at least 75% sequence identity to the sequence of SEQ ID NO: 286.

In some embodiments, the multispecific molecule further comprises a third domain comprising an antigen binding domain. In some embodiments, the antigen binding domain of the third domain binds to a tumor associated antigen (TAA). In some embodiments, the TAA is a tumor antigen, a stromal antigen, or a hematological antigen. In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17. Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, a actinin-4, TRP1/gp75, TRP2, gp100, Melan-A/MART1, gangliosides, WT1. EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the second domain comprises an antigen binding domain.

In some embodiments, the multispecific molecule comprises a single TRAV1-2-, TRBV20-, TRBV6-, or TRBV20- and TRBV6-binding moiety. In some embodiments, the antigen binding domain of the second domain binds to TRAV1-2, TRBV20, TRBV6, or both TRBV20 and TRBV6. In some embodiments, the first domain binds to TRAV1-2 and the antigen binding domain of the second domain binds to TRAV1-2. In some embodiments, the first domain binds to TRBV20 and the antigen binding domain of the second domain binds to TRBV20. In some embodiments, the first domain binds to TRBV6 and the antigen binding domain of the second domain binds to TRBV6. In some embodiments, the first domain binds to both TRBV20 and TRBV6 and the antigen binding domain of the second domain binds to both TRBV20 and TRBV6. In some embodiments, the first domain binds to TRAV1-2 and the antigen binding domain of the second domain binds to TRBV6, the first domain binds to TRAV1-2 and the antigen binding domain of the second domain binds to TRBV20, the first domain binds to TRAV1-2 and the antigen binding domain of the second domain binds to both TRBV20 and TRBV6, the first domain binds to TRBV20 and the antigen binding domain of the second domain binds to TRAV1-2, the first domain binds to TRBV20 and the antigen binding domain of the second domain binds to TRBV6, the first domain binds to TRBV20 and the antigen binding domain of the second domain binds to both TRBV20 and TRBV6, the first domain binds to TRBV6 and the antigen binding domain of the second domain binds to TRAV1-2, the first domain binds to TRBV6 and the antigen binding domain of the second domain binds to TRBV20, the first domain binds to TRBV6 and the antigen binding domain of the second domain binds to both TRBV20 and TRBV6, the first domain binds to both TRBV20 and TRBV6 and the antigen binding domain of the second domain binds to TRAV1-2, the first domain binds to both TRBV20 and TRBV6 and the antigen binding domain of the second domain binds to TRBV20, or the first domain binds to both TRBV20 and TRBV6 and the antigen binding domain of the second domain binds to TRBV6.

In some embodiments, the antigen binding domain of the second domain binds to a tumor associated antigen (TAA). In some embodiments, the TAA is a tumor antigen, a stromal antigen, or a hematological antigen. In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, a actinin-4, TRP1/gp75, TRP2, gp100, Melan-A/MART1, gangliosides, WT1. EphA3, Epidermal growth factor receptor (EGFR), MART-2, MART-1, MUC1, MUC2, MUM1. MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the multispecific molecule further comprises a third domain comprising a cytokine. In some embodiments, the cytokine is any one selected from the group consisting of an interleukin-2 (IL-2) molecule or functional fragment or variant thereof, an interleukin-7 (IL-7) molecule or functional fragment or variant thereof, an interleukin-12 (IL-12) molecule or functional fragment or variant thereof, an interleukin-15 (IL-15) molecule or functional fragment or variant thereof, an interleukin-18 (IL-18) molecule or functional fragment or variant thereof, an interleukin-21 (IL-21) molecule or functional fragment or variant thereof, an interferon gamma molecule or functional fragment or variant thereof, and any combination thereof.

In some embodiments, the multispecific molecule comprises a first polypeptide and a second polypeptide, wherein the first polypeptide and the second polypeptide are non-contiguous, wherein (i) the first polypeptide comprises a first portion of a dimerization module linked to (a) the first domain that binds to TRAV1-2, TRBV20, TRBV6, or both TRBV20 and TRBV6, wherein the first domain that binds to TRAV1-2, TRBV20, TRBV6, or both TRBV20 and TRBV6 comprises a single chain variable fragment (scFv), or a single domain antibody (sdAb), or (b) a first portion of the first domain that binds to TRAV1-2, TRBV20, TRBV6, or both TRBV20 and TRBV6, wherein the first portion of the first domain that binds to TRAV1-2, TRBV20, TRBV6, or both TRBV20 and TRBV6 comprises a heavy chain variable domain (VH), wherein when the first polypeptide comprises the first portion of the first domain that binds to TRAV1-2, TRBV20, TRBV6, or both TRBV20 and TRBV6, the multispecific molecule further comprises a third polypeptide comprising a second portion of the first domain that binds to TRAV1-2, TRBV20, TRBV6, or both TRBV20 and TRBV6, wherein the second portion of the first domain that binds to TRAV1-2, TRBV20, TRBV6, or both TRBV20 and TRBV6 comprises a light chain variable domain (VL), wherein the third polypeptide is non-contiguous with the first polypeptide and the second polypeptide; and (ii) the second polypeptide comprises a second portion of the dimerization module, wherein (a) the cytokine or functional fragment or variant thereof is covalently linked to the first polypeptide, the second polypeptide, or the third polypeptide, or (b) the antigen binding domain is covalently linked to the first polypeptide, the second polypeptide, or the third polypeptide.

In some embodiments, the first polypeptide comprises the first portion of the first domain that binds to TRAV1-2, TRBV20, TRBV6, or both TRBV20 and TRBV6, and wherein (i) the first portion of the first domain that binds to TRAV1-2, TRBV20, TRBV6, or both TRBV20 and TRBV6 is linked to the N-terminus of the first polypeptide comprising the first portion of the dimerization module; and (ii) the second polypeptide is linked to the C-terminus of a cytokine or a functional fragment or variant thereof.

In some embodiments, the multispecific molecule comprises a first polypeptide and a second polypeptide, wherein the first polypeptide and the second polypeptide are non-contiguous, wherein the first polypeptide comprises each of the following linked together: (i) a first portion of a dimerization module; and (ii) (a) a third domain comprising an antigen binding domain, wherein the third domain comprises a single chain variable fragment (scFv), or a single domain antibody (sdAb), or (b) a first portion of the third domain, wherein the first portion of the third domain comprises a heavy chain variable domain (VH), wherein when the first polypeptide comprises the first portion of the third domain, the multispecific molecule further comprises a third polypeptide comprising a second portion of the third domain, wherein the second portion of the third domain comprises a light chain variable domain (VL), wherein the third polypeptide is non-contiguous with the first polypeptide and the second polypeptide; and wherein the second polypeptide comprises each of the following linked together: (i) a second portion of the dimerization module; (ii) the second domain, wherein the second domain comprises a cytokine; (iii) (a) the first domain, wherein the first domain comprises a single chain variable fragment (scFv), or a single domain antibody (sdAb), or (b) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: (i) a first portion of a dimerization module; (ii) (a) the second domain comprising an antigen binding domain, wherein the second domain comprises a single chain variable fragment (scFv), or a single domain antibody (sdAb), or (b) a first portion of the second domain, wherein the first portion of the second domain comprises a heavy chain variable domain (VH), wherein when the first polypeptide comprises the first portion of the second domain, the multispecific molecule further comprises a third polypeptide comprising a second portion of the second domain, wherein the second portion of the second domain comprises a light chain variable domain (VL), wherein the third polypeptide is non-contiguous with the first polypeptide and the second polypeptide; and wherein the second polypeptide comprises each of the following linked together: (i) a second portion of the dimerization module; and (ii) (a) the first domain, wherein the first domain comprises a single chain variable fragment (scFv), or a single domain antibody (sdAb), or (b) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: (i) a first portion of a dimerization module; (ii) (a) the first domain, wherein the first domain comprises a single chain variable fragment (scFv), or a single domain antibody (sdAb), or (b) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and (iii) (a) the second domain, wherein the second domain comprises the cytokine or a functional fragment or variant thereof, or a single chain variable fragment (scFv), or a single domain antibody (sdAb), or (b) a first portion of the second domain, wherein the second domain comprises the antigen binding domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide; wherein the second polypeptide comprises each of the following linked together: (i) a second portion of the dimerization module; (ii) (a) an additional first domain, wherein the additional first domain comprises a single chain variable fragment (scFv), or a single domain antibody (sdAb), or (b) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and (iii) (a) an additional second domain, wherein the second domain comprises the cytokine or a functional fragment or variant thereof, or a single chain variable fragment (scFv), or a single domain antibody (sdAb), or (b) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide, and/or the fifth polypeptide, the sixth polypeptide is non-contiguous with the third polypeptide, the fourth polypeptide, and the fifth polypeptide.

In some embodiments, the first portion of the dimerization module comprises a first immunoglobulin constant region (Fc region) and the second portion of the dimerization module comprises a second Fc region. In some embodiments, the first Fc region, the second Fc region, or a combination thereof is selected from the group consisting of IgG1, IgG2, IgG3, IgG4, IgAQ1, IgA2, IgJ, IgM, IgD, IgE, and any fragment thereof. In some embodiments, the first Fc region, the second Fc region, or a combination thereof comprises an IgGI constant region comprising one or more amino acid substitutions selected from the group consisting of N297A, K322A, L234A and L235A. In some embodiments, the first Fc region, the second Fc region, or a combination thereof comprises a sequence having at least 75% sequence identity to the sequence of SEQ ID NO: 40, SEQ ID NO: 42, SEQ ID NO: 204, SEQ ID NO: 205, SEQ ID NO: 41, SEQ ID NO:206, or SEQ ID NO: 207.

In some embodiments, the first domain that binds to TRAV1-2, TRBV20, TRBV6, or both TRBV20 and TRBV6, the second domain that binds to TRAV1-2, TRBV20, TRBV6, or both TRBV20 and TRBV6, or both comprises a Fab, a F(ab′)2, an Fv, a single chain Fv (scFv), a single domain antibody, a diabody (dAb), a camelid antibody, and a combination thereof. In some embodiments, the first domain that binds to TRAV1-2, TRBV20. TRBV6, or both TRBV20 and TRBV6, the second domain that TRAV1-2, TRBV20, TRBV6, or both TRBV20 and TRBV6, or both comprises a Fab or an scFv. In some embodiments, the first domain that binds to (i) TRAV1-2 and comprises a VH comprising heavy chain complementarity determining regions (HC CDR1-3) of SEQ ID NOs: 197-199, respectively, and a VL comprising light chain complementarity determining regions LC CDR1-3 of SEQ ID NOs: 200-202, respectively; (ii) TRBV6 and comprises a VH comprising (a) a VH comprising: HC CDR1 of SEQ ID NO: 3, 45, 15, 164, 174, 229, 234, 314, 317, or 319, HC CDR2 of SEQ ID NO: 4, 16, 165, 170, 175, 178, 181, 365, 192, 315, or 318, HC CDR3 of SEQ ID NO: 5, or 316, or any combination thereof; (b) a VL comprising: LC CDR1 of SEQ ID NO: 6, 166, 171, 364, 176, 179, 182, 186, 320, or 323, LC CDR2 of SEQ ID NO: 7, 167, 188, 299, or 321, LC CDR3 of SEQ ID NO: 8, 190, or 322, or any combination thereof: or (c) any combination thereof: or (iii) TRBV20 and comprises a VH comprising complementarity determining regions (HC CDR1-3) of SEQ ID NOs: 339-341, respectively, and a VL comprising LC CDR1-3 of SEQ ID NOs: 342-344, respectively. In some embodiments, the first domain binds to TRAV1-2. In some embodiments, the composition further comprises a second multispecific molecule, wherein the second multispecific molecule comprises a domain that binds to TRBV20, TRBV6, or both TRBV20 and TRBV6. In some embodiments, the first domain binds to TRBV20. In some embodiments, the composition further comprises a second multispecific molecule, wherein the second multispecific molecule comprises a domain that binds to TRAV1-2, TRBV6, or both TRBV20 and TRBV6. In some embodiments, the first domain binds to TRBV6. In some embodiments, the composition further comprises a second multispecific molecule, wherein the second multispecific molecule comprises a domain that binds to TRAV1-2, TRBV20, or both TRBV20 and TRBV6. In some embodiments, the first domain binds to both TRBV20 and TRBV6.

In some embodiments, the composition further comprises a second multispecific molecule, wherein the second multispecific molecule comprises a fourth domain that binds to TRAV1-2, TRBV20, or TRBV6.

In some embodiments, the second multispecific molecule comprises a fifth domain, and wherein the fifth domain comprises (i) a cytokine or a functional fragment or variant thereof, or (ii) an antigen binding domain. In some embodiments, the fifth domain comprises a cytokine.

In some embodiments, the second multispecific molecule comprises a sixth domain comprising an antigen binding domain.

In some embodiments, the antigen binding domain of the six domain binds to TRAV1-2, TRBV20, TRBV6, or both TRBV20 and TRBV6. In some embodiments, the antigen binding domain of the six domain binds to a TAA.

In some embodiments, the fifth domain comprises an antigen binding domain.

In some embodiments, the second multispecific molecule comprises a sixth domain comprising a cytokine.

In some embodiments, the antigen binding domain binds of the fifth domain binds to TRAV1-2, TRBV20, TRBV6, or both TRBV20 and TRBV6.

In some embodiments, the antigen binding domain binds of the fifth domain binds to a TAA.

Disclosed herein, in some aspects, is a pharmaceutical composition comprising the composition of any one of the preceding embodiments.

Disclosed herein, in some aspects, is a method of expanding a population of cells comprising a MAIT cell comprising contacting the population of cells comprising the MAIT cell with the composition of any one of preceding embodiments.

Provided herein is a method of expanding a population of cells comprising a MAIT cell comprising contacting the population of cells comprising the MAIT cell with a composition comprising a molecule, wherein the molecule comprises a first domain, and wherein the first domain binds to a target molecule, wherein the target molecule is TRAV1-2, TRBV20, TRBV6, or both TRBV20 and TRBV6, thereby expanding the MAIT cell.

In some embodiments, the method comprises expanding the MAIT cell at least 2-fold. In some embodiments, the method comprises expanding the MAIT cell at least 5-fold or at least 10-fold.

In some embodiments, the method comprises selectively expanding the MAIT cell.

In some embodiments, the method comprises expanding the MAIT cell, thereby producing an expanded cell population, wherein at least 1% of the cells in the expanded population of cells are MAIT cells. In some embodiments, the method comprises expanding the MAIT cell, thereby producing an expanded cell population, wherein at most 20% of the cells in the expanded population of cells are MAIT cells.

Disclosed herein, in some aspects, is a method of activating a population of cells comprising a MAIT cell comprising contacting the population of cells comprising the MAIT cell with the composition of any one of the preceding embodiments.

Provided herein is a method of activating a population of cells comprising a MAIT cell comprising contacting the population of cells comprising the MAIT cell with a composition comprising a molecule, wherein the molecule comprises a first domain and a second domain, wherein the first domain binds to a target molecule, wherein the target molecule is TRAV1-2, TRBV20, TRBV6, or both TRBV20 and TRBV6, and wherein the second domain comprises a cytokine or a functional fragment or variant thereof, thereby activating the MAIT cell.

In some embodiments, the method specifically activates the MAIT cell of the population of cells.

In some embodiments, the method comprises activating the MAIT cell, thereby producing an activated cell population, wherein the activated cell population produces: (i) a higher level of IL-2, IL-17, IL-22, IL-26, IFN-7, or TNF compared to the level of the IL-2, IL-17, IL-22, IL-26, IFN-γ, or TNF produced by a cell population produced by contacting the population of cells comprising the MAIT cell with an anti-CD3 antibody at the same concentration, an anti-TRBV antibody that does not bind TRBV20. TRBV6 or both TRBV20 and TRBV6 at the same concentration, or an anti-TRAV antibody that does not bind TRAV1-2 at the same concentration according to a cytokine release assay, and/or (ii) a lower level of IL-4, IL-10, or IL-12 compared to the level of the IL-4, IL-10, or IL-12 produced by a cell population produced by contacting the population of cells comprising the MAIT cell with an anti-CD3 antibody at the same concentration, an anti-TRBV antibody that does not bind TRBV20, TRBV6 or both TRBV20 and TRBV6 at the same concentration, or an anti-TRAV antibody that does not bind TRAV1-2 at the same concentration according to a cytokine release assay.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-CD3 antibody at the same concentration.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRBV antibody that does not bind TRBV20 or TRBV6 at the same concentration.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRAV antibody that does not bind TRAV1-2 at the same concentration.

In some embodiments, the activated cell population produces a lower level of IL-4, IL-10, or IL-12 compared to the level of the IL-4, IL-10, or IL-12 produced by a cell population produced by contacting the population of cells comprising the MAIT cell with an anti-CD3 antibody at the same concentration.

In some embodiments, the activated cell population produces a lower level of IL-4, IL-10, or IL-12 compared to the level of the IL-4, IL-10, or IL-12 produced by a cell population produced by contacting the population of cells comprising the MAIT cell with an anti-TRBV antibody that does not bind TRBV20 or TRBV6 at the same concentration.

In some embodiments, the activated cell population produces a lower level of IL-4, IL-10, or IL-12 compared to the level of the IL-4, IL-10, or IL-12 produced by a cell population produced by contacting the population of cells comprising the MAIT cell with an anti-TRAV antibody that does not bind TRAV1-2 at the same concentration.

In some embodiments, the MAIT cell expresses TRAV1-2, TRBV20, TRBV6, or both TRBV20 and TRBV6.

In some embodiments, the MAIT cell are CD45RA-CD45RO+CD95HiCD62LLoCD44Hi.

In some embodiments, the method is performed in vivo. In some embodiments, the method is performed ex vivo.

Disclosed herein, in some aspects, is a method of treating a disease or condition in a subject in need thereof comprising administering to the subject a therapeutically effective amount of the composition of any one of the preceding embodiments or pharmaceutical composition of any of the preceding embodiments.

Provided herein is a method of treating a disease or condition in a subject in need thereof comprising administering to the subject a therapeutically effective amount of a composition comprising a molecule to activate or expand a MAIT cell of the subject, wherein the molecule comprises a first domain and a second domain, wherein the first domain binds to TRAV1-2, TRBV20, TRBV6, or both TRBV20 and TRBV6, and wherein the second domain comprises a cytokine or a functional fragment or variant thereof.

In some embodiments, the method further comprises administering a third therapeutic agent or therapy to the subject. In some embodiments, the third therapeutic agent or therapy comprises a chemotherapeutic agent, a biologic agent, a hormonal therapy, radiation, or surgery. In some embodiments, the third therapeutic agent or therapy is administered in combination with one or more of the molecules disclosed herein sequentially, simultaneously, or concurrently.

In some embodiments, the disease or condition is a cancer. In some embodiments, the cancer is a solid tumor, a hematological cancer, a metastatic cancer, a soft tissue tumor, or any combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and any combination thereof. In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof. In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and any combination thereof. In some embodiments, the T-cell lymphoma is peripheral T-cell lymphoma. In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2. Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, O-catenin, BRCA1/2, CDK4, CML66, Fibronectin, p53, Ras, TGF-B receptor, AFP, ETA, MAGE, MUC-1, CA-125, BAGE, GAGE, NY-ESO-1, O-catenin, CDK4, CDC27, α actinin-4, TRP1/gp75, TRP2, gpoo, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1. OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the disease or condition is an autoimmune disease. In some embodiments, the autoimmune diseases is selected from the group consisting of multiple sclerosis, Crohn's disease, Sjögren's syndrome, celiac disease, diabetes, arthritis, and Lupus, systemic sclerosis, osteopetrosis, inherited metabolic disorders, adrenoleukodystrophy, amegakaryocytic thrombocytopenia, sickle cell disease, severe congenital immunodeficiency. Griscelli syndrome type 11, Hurler syndrome, Kostmann syndrome, Krabbe disease, metachromatic leukodystrophy, thalassemia, hemophagocytic lymphohistiocytosis, and Wiskott-Aldrich syndrome.

In some embodiments, the disease or condition is an infection. In some embodiments, the infection is selected from the group consisting of a S. parathyphi infection, a Bacteroidetes infection, a Proteobacteria infection, a M. tuberculosis infection, a respiratory virus infection, a SARS-CoV-2 infection, yellow fever, influenza, a HIV infection, a Cytomegalovirus infection, and an Epstein-Barr Virus infection.

### Part II

In one aspect, provided herein is a composition comprising a multispecific molecule, wherein the multispecific molecule comprises:
(a) a first domain that binds to a first target molecule, wherein the first target molecule is T cell receptor beta variable 25 (TRBV25) or T cell receptor alpha variable 10 (TRAV10), and
(b) a second domain that binds to a second target molecule, wherein the second domain comprises (i) a cytokine or a functional fragment or variant thereof, or (ii) an antigen binding domain.

In another aspect, provided herein is a pharmaceutical composition comprising the composition disclosed herein.

In another aspect, provided herein is a method of expanding a population of cells comprising an invariant Natural Killer T (iNKT) cell comprising contacting the population of cells comprising the iNKT cell with the composition disclosed herein.

In another aspect, provided herein is a method of activating a population of cells comprising an invariant Natural Killer T (iNKT) cell comprising contacting the population of cells comprising the iNKT cell with the composition disclosed herein.

In another aspect, provided herein is a method of expanding a population of cells comprising an invariant Natural Killer T (iNKT) cell comprising contacting the population of cells comprising the iNKT cell with a composition comprising a molecule comprising a first domain that binds to a first target molecule, thereby expanding the iNKT cell, wherein the first target molecule is TRBV25 or TRAV10.

In another aspect, provided herein is a method of activating a population of cells comprising an invariant Natural Killer T (iNKT) cell comprising contacting a population of cells comprising the iNKT cell with a composition comprising a molecule comprising a first domain that binds to a first target molecule, thereby activating the iNKT cell, wherein the first target molecule is TRBV25 or TRAV10.

In another aspect, provided herein is a method of treating a disease or condition in a subject in need thereof comprising administering to the subject a therapeutically effective amount of the composition of any composition or pharmaceutical composition disclosed herein.

In another aspect, provided herein is a method of treating a disease or condition in a subject in need thereof comprising administering to the subject a therapeutically effective amount of a composition comprising a molecule comprising a first domain that binds to a first target molecule, wherein the first target molecule is TRBV25 or TRAV10.

In another aspect, provided herein is a composition comprising a multispecific molecule, wherein the multispecific molecule comprises
(a) a first polypeptide and a second polypeptide, wherein the first polypeptide and the second polypeptide are non-contiguous, and
(b) a first domain that binds to a first target molecule, wherein the first target molecule is a TRBV or a TRAV, and
(c) a second domain that binds to a second target molecule, wherein the second domain comprises an antigen binding domain;
wherein the first polypeptide comprises each of the following linked together:
   (i) a first portion of a dimerization module;
   (ii) (A) the first domain, wherein the first domain comprises a single chain variable fragment (scFv), or a single domain antibody (sdAb), or
      (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and
   (iii) (A) the second domain, wherein the second domain comprises a single chain variable fragment (scFv), or a single domain antibody (sdAb), or
      (B) a first portion of the second domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide;
wherein the second polypeptide comprises a second portion of the dimerization module.

In another aspect, provided herein is a composition comprising a multispecific molecule, wherein the multispecific molecule comprises
(a) a first polypeptide and a second polypeptide, wherein the first polypeptide and the second polypeptide are non-contiguous, and
(b) a first domain that binds to a first target molecule, wherein the first target molecule is a TRBV or a TRAV, and
(c) a second domain that binds to a second target molecule, wherein the second domain comprises (i) a cytokine or a functional fragment or variant thereof, or (ii) an antigen binding domain:
wherein the first polypeptide comprises each of the following linked together:
   (i) a first portion of a dimerization module;
   (ii) (A) the first domain, wherein the first domain comprises a single chain variable fragment (scFv), or a single domain antibody (sdAb), or
      (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and
   (iii) (A) the second domain, wherein the second domain comprises the cytokine or a functional fragment or variant thereof, or a single chain variable fragment (scFv), or a single domain antibody (sdAb), or
      (B) a first portion of the second domain, wherein the second domain comprises the antigen binding domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide;
wherein the second polypeptide comprises each of the following linked together:
   (i) a second portion of the dimerization module;
   (ii) (A) an additional first domain, wherein the additional first domain comprises a single chain variable fragment (scFv), or a single domain antibody (sdAb), or
      (B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and
   (iii) (A) an additional second domain, wherein the second domain comprises the cytokine or a functional fragment or variant thereof, or a single chain variable fragment (scFv), or a single domain antibody (sdAb), or
      (B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide, and/or the fifth polypeptide, the sixth polypeptide is non-contiguous with the third polypeptide, the fourth polypeptide, and the fifth polypeptide.

### Part III

Provided herein is a composition comprising a multispecific molecule, wherein the multispecific molecule comprises: a), a first domain that binds to a first target molecule, wherein the first target molecule is a T cell receptor alpha variable chain 1-2 (TRAV1-2), a T cell receptor beta variable chain 6-2 (TRBV6-2), a T cell receptor beta variable chain 4-1 (TRBV4-1), or a T cell receptor alpha variable chain 17 (TRAV17); and b), a second domain that binds to a second target molecule, wherein the second domain comprises (i) a cytokine or a functional fragment or variant thereof, or (ii) an antigen binding domain.

In some embodiments, the composition further comprises a T cell, the T cell binds to CD1b or CD1c presented antigens.

In some embodiments, the multispecific molecule is bound to the T cell.

In some embodiments, the multispecific molecule is an agonist of TRAV1-2, TRBV6-2, TRBV4-1 or TRAV17.

In some embodiments, the multispecific molecule is an antagonist of TRAV1-2, TRBV6-2, TRBV4-1 or TRAV17.

In some embodiments, the first domain binds to TRAV1-2.

In some embodiments, the TRAV1-2 is TRAV1-2*01, TRAV1-2*02, or TRAV1-3*03.

In some embodiments, the first domain binds to TRBV6-2.

In some embodiments, the TRBV6-2 is TRBV6-2*01.

In some embodiments, the first domain binds to TRBV4-1.

In some embodiments, the TRBV4-1 is TRBV4-1*01.

In some embodiments, the first domain binds to TRAV17.

In some embodiments, the TRAV17 is TRAV17*01.

In some embodiments, the second domain comprises a cytokine.

In some embodiments, the cytokine comprises IL-2, IL-7, IL-12, IL-15, IL-18, IL-21, or any functional fragment or variant thereof.

In some embodiments, the cytokine comprises IL-2 or a functional fragment or variant thereof.

In some embodiments, the IL-2 functional fragment or variant thereof comprises a sequence having at least 75% sequence identity to the sequence of SEQ ID NO: 1268.

In some embodiments, the cytokine comprises IL-7 or a functional fragment or variant thereof.

In some embodiments, the IL-7 or the functional fragment or variant thereof comprises a sequence having at least 75% sequence identity to the sequence of SEQ ID NO: 1273.

In some embodiments, the cytokine comprises IL-12 or a functional fragment or variant or variant thereof.

In some embodiments, the IL-12 or the functional fragment or variant thereof comprises a sequence having at least 75% sequence identity to the sequence of SEQ ID NO: 1276.

In some embodiments, the cytokine comprises IL-15 or a functional fragment or variant or variant thereof.

In some embodiments, the IL-15 or the functional fragment or variant thereof comprises a sequence having at least 75% sequence identity to the sequence of SEQ ID NO: 1297.

In some embodiments, the cytokine comprises IL-18 or a functional fragment or variant thereof.

In some embodiments, the IL-18 or the functional fragment or variant thereof comprises a sequence having at least 75% sequence identity to the sequence of SEQ ID NO: 1285.

In some embodiments, the cytokine comprises IL-21 or a functional fragment or variant thereof.

In some embodiments, the IL-21 or the functional fragment or variant thereof comprises a sequence having at least 75% sequence identity to the sequence of SEQ ID NO: 1286.

In some embodiments, the multispecific molecule further comprises a third domain comprising an antigen binding domain.

In some embodiments, the antigen binding domain of the third domain binds to a tumor associated antigen (TAA).

In some embodiments, the TAA is a tumor antigen, a stromal antigen, or a hematological antigen.

In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the second domain comprises an antigen binding domain.

In some embodiments, the multispecific molecule comprises a single TRAV1-2, TRBV6-2, TRBV4-1 or TRAV17 binding moiety.

In some embodiments, the antigen binding domain of the second domain binds to TRAV1-2, TRBV6-2, TRBV4-1 or TRAV17.

In some embodiments, wherein the first domain and the antigen binding domain of the second domain bind to: TRAV1-2 and TRAV1-2, respectively; TRAV1-2 and TRBV6-2, respectively: TRAV1-2 and TRBV4-1, respectively; TRAV1-2 and TRAV17, respectively; TRBV6-2 and TRAV1-2, respectively: TRBV6-2 and TRBV6-2, respectively; TRBV6-2 and TRBV4-1, respectively: TRBV6-2 and TRAV17, respectively; TRBV4-1 and TRAV1-2, respectively; TRBV4-1 and TRBV6-2, respectively; TRBV4-1 and TRBV4-1, respectively; TRBV4-1 and TRAV17, respectively; TRAV17 and TRAV1-2, respectively: TRAV17 and TRBV6-2, respectively; TRAV17 and TRBV4-1, respectively; TRAV17 and TRAV17, respectively.

In some embodiments, the antigen binding domain of the second domain binds to a tumor associated antigen (TAA).

In some embodiments, the TAA is a tumor antigen, a stromal antigen, or a hematological antigen.

In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the multispecific molecule further comprises a third domain comprising a cytokine.

In some embodiments, the multispecific molecule comprises a first polypeptide and a second polypeptide, wherein the first polypeptide and the second polypeptide are non-contiguous, wherein the first polypeptide comprises a first portion of a dimerization module linked to (A) the first domain that binds to TRAV1-2, TRBV6-2, TRBV4-1 or TRAV17, wherein the first domain that binds to TRAV1-2, TRBV6-2, TRBV4-1 or TRAV17 comprises a single chain variable fragment (scFv), or a single domain antibody (sdAb), or (B) a first portion of the first domain that binds to TRAV1-2, TRBV6-2, TRBV4-1 or TRAV17, wherein the first portion of the first domain that binds to TRAV1-2, TRBV6-2, TRBV4-1 or TRAV17 comprises a heavy chain variable domain (VH), b. wherein when the first polypeptide comprises the first portion of the first domain that binds to TRAV1-2, TRBV6-2, TRBV4-1 or TRAV17, the multispecific molecule further comprises a third polypeptide comprising a second portion of the first domain that binds to TRAV1-2, TRBV6-2, TRBV4-1 or TRAV17, wherein the second portion of the first domain that binds to TRAV1-2, TRBV6-2, TRBV4-1 or TRAV17 comprises a light chain variable domain (VL), wherein the third polypeptide is non-contiguous with the first polypeptide and the second polypeptide; and (ii) the second polypeptide comprises a second portion of the dimerization module; wherein (a) the cytokine or functional fragment or variant thereof is covalently linked to the first polypeptide, the second polypeptide, or the third polypeptide, or (b) the antigen binding domain is covalently linked to the first polypeptide, the second polypeptide, or the third polypeptide.

In some embodiments, the first polypeptide comprises the first portion of the first domain that binds to TRAV1-2, TRBV6-2, TRBV4-1 or TRAV17, and wherein (i) the first portion of the first domain that binds to TRAV1-2, TRBV6-2, TRBV4-1 or TRAV17 is linked to the N-terminus of the first polypeptide comprising the first portion of the dimerization module; and (ii) the second polypeptide is linked to the C-terminus of a cytokine or a functional fragment or variant thereof.

In some embodiments, the multispecific molecule comprises a first polypeptide and a second polypeptide, wherein the first polypeptide and the second polypeptide are non-contiguous, wherein the first polypeptide comprises each of the following linked together: (i) a first portion of a dimerization module; and (ii)(A) a third domain comprising an antigen binding domain, wherein the third domain comprises a single chain variable fragment (scFv), or a single domain antibody (sdAb), or (B) a first portion of the third domain, wherein the first portion of the third domain comprises a heavy chain variable domain (VH), wherein when the first polypeptide comprises the first portion of the third domain, the multispecific molecule further comprises a third polypeptide comprising a second portion of the third domain, wherein the second portion of the third domain comprises a light chain variable domain (VL), wherein the third polypeptide is non-contiguous with the first polypeptide and the second polypeptide; and wherein the second polypeptide comprises each of the following linked together: (i) a second portion of the dimerization module; (ii) the second domain, wherein the second domain comprises a cytokine; (iii)(A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: (i) a first portion of a dimerization module; (ii)(A) the second domain comprising an antigen binding domain, wherein the second domain comprises a single chain variable fragment (scFv), or a single domain antibody (sdAb), or (B) a first portion of the second domain, wherein the first portion of the second domain comprises a heavy chain variable domain (VH), wherein when the first polypeptide comprises the first portion of the second domain, the multispecific molecule further comprises a third polypeptide comprising a second portion of the second domain, wherein the second portion of the second domain comprises a light chain variable domain (VL), wherein the third polypeptide is non-contiguous with the first polypeptide and the second polypeptide; and wherein the second polypeptide comprises each of the following linked together: (i) a second portion of the dimerization module; and (ii)(A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: a.(i) a first portion of a dimerization module; (ii)(A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and b.(iii) (A) the second domain, wherein the second domain comprises the cytokine or a functional fragment or variant thereof, or a single chain variable fragment (scFv), or a single domain antibody (sdAb), or (B) a first portion of the second domain, wherein the second domain comprises the antigen binding domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide; wherein the second polypeptide comprises each of the following linked together: (i) a second portion of the dimerization module; (ii)(A) an additional first domain, wherein the additional first domain comprises a single chain variable fragment (scFv), or a single domain antibody (sdAb), or ii.(B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and c.(iii) (A) an additional second domain, wherein the second domain comprises the cytokine or a functional fragment or variant thereof, or a single chain variable fragment (scFv), or a single domain antibody (sdAb), or (B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide, and/or the fifth polypeptide, the sixth polypeptide is non-contiguous with the third polypeptide, the fourth polypeptide, and the fifth polypeptide.

In some embodiments, the first portion of the dimerization module comprises a first immunoglobulin constant region (Fc region) and the second portion of the dimerization module comprises a second Fc region.

In some embodiments, the first Fc region, the second Fc region, or a combination thereof is selected from the group consisting of IgG1, IgG2, IgG3, IgG4, IgAQ1, IgA2, IgJ, IgM, IgD, IgE, and any fragment thereof.

In some embodiments, the first Fc region, the second Fc region, or a combination thereof comprises an IgGI constant region comprising one or more amino acid substitutions selected from the group consisting of N297A, K322A, L234A and L235A.

In some embodiments, the first Fc region, the second Fc region, or a combination thereof comprises a sequence having at least 75% sequence identity to the sequence SEQ ID NO: 1040, SEQ ID NO: 1042, SEQ ID NO: 1204, SEQ ID NO: 1205, SEQ ID NO: 1041, SEQ ID NO: 1206, or SEQ ID NO: 1207.

In some embodiments, the first domain that binds to TRAV1-2, TRBV6-2, TRBV4-1 or TRAV17, a second domain that binds to TRAV1-2, TRBV6-2, TRBV4-1 or TRAV17, or any combination thereof comprises a Fab, a F(ab′)2, an Fv, a single chain Fv (scFv), a single domain antibody, a diabody (dAb), a camelid antibody, and a combination thereof.

In some embodiments, the first domain that binds to TRAV1-2, TRBV6-2, TRBV4-1 or TRAV17, a second domain that binds to TRAV1-2, TRBV6-2, TRBV4-1 or TRAV17, or any combination thereof comprises a Fab or an scFv.

In some embodiments, the first domain that binds to (i) TRAV1-2 comprises a VH comprising complementarity determining regions (VHCDR1-3) of SEQ ID NOs: 1197-1199, respectively, and a VL comprising complementarity determining regions (VLCDR1-3) of SEQ ID NOs: 1200-1202, respectively; or (ii) TRBV4-1 comprises: (a) a VH comprising complementarity determining regions (VH-CDR1-3) that comprise the sequences of: SEQ ID NOs: 1208, 1209, 1210, respectively; SEQ ID NOs: 1211, 1212, 1210, respectively; or SEQ ID NOs: 1213, 1209, 1210, respectively; (b) a VL comprising complementarity determining regions (VLCDR1-3) that comprise the sequences of: SEQ ID NOs: 1214, 1215, 1216, respectively; or SEQ ID NOs: 1217, 1215, 1216, respectively; or (c) any combination thereof; or (iii) TRBV6-2 comprises: (a) a VH comprising complementarity determining regions (VHCDR1-3) that comprise the sequences of: SEQ ID NOs: 1003, 1004, 1005, respectively; SEQ ID NOs: 1045, 1004, 1005, respectively: SEQ ID NOs: 1015, 1016, 1005, respectively; SEQ ID NOs: 1164, 1165, 1005, respectively: SEQ ID NOs: 1164, 1170, 1005, respectively; SEQ ID NOs: 1174, 1175, 1005, respectively; SEQ ID NOs: 1164, 1178, 1005, respectively; SEQ ID NOs: 1174, 1171, 1005, respectively; SEQ ID NOs: 1174, 1314, 1005, respectively; SEQ ID NOs: 1174, 1192, 1005, respectively; SEQ ID NOs: 1164, 1175, 1005, respectively; SEQ ID NOs: 1229, 1165, 1005, respectively; SEQ ID NOs: 1234, 1165, 1005, respectively; or SEQ ID NOs: 1164, 1165, 1167, respectively; (b) a VL comprising complementarity determining regions (VLCDR1-3) that comprise the sequences of: SEQ ID NOs: 1016, 1007, 1008, respectively; SEQ ID NOs: 1166, 1167, 1168, respectively: SEQ ID NOs: 1171, 1167, 1008, respectively; SEQ ID NOs: 1313, 1167, 1008, respectively; SEQ ID NOs: 1176, 1167, 1008, respectively; SEQ ID NOs: 1179, 1167, 1008, respectively; SEQ ID NOs: 1172, 1167, 1008, respectively; SEQ ID NOs: 1182, 1167, 1008, respectively; SEQ ID NOs: 1186, 1167, 1008, respectively; SEQ ID NOs: 1176, 1188, 1008, respectively; SEQ ID NOs: 1176, 1167, 1190, respectively; SEQ ID NOs: 1177, 1299, 1008, respectively; SEQ ID NOs: 1171, 1299, 1008, respectively; or SEQ ID NOs: 1170, 1171, 1008, respectively; or (c) any combination thereof.

In some embodiments, the first domain that binds to (i) TRAV1-2 comprises a VH comprising a sequence having at least 75% sequence identity to the sequence of SEQ ID NO: 1195, and a VL comprising a sequence having at least 75% sequence identity to the sequence of SEQ ID NO: 1196; or (ii) TRBV4-1 comprises: (a) a VH comprising a sequence having at least 75% sequence identity to the sequence of SEQ ID NO: 1219, SEQ ID NO: 1220, SEQ ID NO: 1221, SEQ ID NO: 1222, or SEQ ID NO: 1223; (b) a VL comprising a sequence having at least 75% sequence identity to the sequence of SEQ ID NO: 1218, SEQ ID NO: 1224, SEQ ID NO: 1225, SEQ ID NO: 1226, SEQ ID NO: 1227; or (c) any combination thereof; or (iii) TRBV6-2 comprises: (a) a VH comprising a sequence having at least 75% sequence identity to the sequence of: SEQ ID NO: 1001; SEQ ID NO: 1009; SEQ ID NO: 1025; SEQ ID NO: 1027; SEQ ID NO: 1029; SEQ ID NO: 1030; SEQ ID NO: 1033; SEQ ID NO: 1036; SEQ ID NO: 1038; SEQ ID NO: 1047; SEQ ID NO: 1050: SEQ ID NO: 1056; SEQ ID NO: 1059; SEQ ID NO: 1063; SEQ ID NO: 1065; SEQ ID NO: 1067; SEQ ID NO: 1070; SEQ ID NO: 1072; SEQ ID NO: 1078; SEQ ID NO: 1080; SEQ ID NO: 1083: SEQ ID NO: 1085; SEQ ID NO: 1088; SEQ ID NO: 1090; SEQ ID NO: 1092: SEQ ID NO: 1094: SEQ ID NO: 1096; SEQ ID NO: 1097; SEQ ID NO: 1100; SEQ ID NO: 1104; SEQ ID NO: 1106; SEQ ID NO: 1108; SEQ ID NO: 1111; SEQ ID NO: 1113: SEQ ID NO: 1115; SEQ ID NO: 1117; SEQ ID NO: 1120; SEQ ID NO: 1122: SEQ ID NO: 1123; SEQ ID NO: 1125; SEQ ID NO: 1168: SEQ ID NO: 1172; SEQ ID NO: 1173; SEQ ID NO: 1177; SEQ ID NO: 1180; SEQ ID NO: 1183; SEQ ID NO: 1185; SEQ ID NO: 1193; SEQ ID NO: 1194; or SEQ ID NO: 1317; (b) a VL comprising a sequence having at least 75% sequence identity to the sequence of: SEQ ID NO: 1002; SEQ ID NO: 1010; SEQ ID NO: 1011; SEQ ID NO: 1024; SEQ ID NO: 1029; SEQ ID NO: 1032: SEQ ID NO: 1035; SEQ ID NO: 1049; SEQ ID NO: 1055; SEQ ID NO: 1058; SEQ ID NO: 1062; SEQ ID NO: 1082: SEQ ID NO: 1087; SEQ ID NO: 1096; SEQ ID NO: 1102: SEQ ID NO: 1110; SEQ ID NO: 1148; SEQ ID NO: 1169; SEQ ID NO: 1173; SEQ ID NO: 1184; SEQ ID NO: 1187; SEQ ID NO: 1189; SEQ ID NO: 1191; SEQ ID NO: 1300; SEQ ID NO: 1303; or (c) any combination thereof.

In some embodiments, the first domain binds to TRAV1-2.

In some embodiments, the composition further comprises a second multispecific molecule the second multispecific molecule comprises a fourth domain that binds to TRBV6-2 TRBV4-1, or TRAV17.

In some embodiments, the first domain binds to TRBV6-2.

In some embodiments, the composition further comprises a second multispecific molecule the second multispecific molecule comprises a fourth domain that binds to TRBV1-2, TRBV4-1, or TRAV17.

In some embodiments, the first domain binds to TRBV4-1.

In some embodiments, the composition further comprises a second multispecific molecule the second multispecific molecule comprises a fourth domain that binds to TRAV1-2, TRBV4-1, or TRAV17.

In some embodiments, the first domain binds to TRAV17.

In some embodiments, the composition further comprises a second multispecific molecule, wherein the second multispecific molecule comprises a fourth domain that binds to TRAV1-2, TRBV6-2, or TRBV4-1.

In some embodiments, the second multispecific molecule comprises a fifth domain, and wherein the fifth domain comprises (i) a cytokine or a functional fragment or variant thereof, or (ii) an antigen binding domain.

In some embodiments, the fifth domain comprises a cytokine.

In some embodiments, the fifth domain comprises an antigen binding domain.

In some embodiments, the second multispecific molecule comprises a sixth domain comprising an antigen binding domain.

In some embodiments, the antigen binding domain of the sixth domain binds to TRAV1-2, TRBV6-2, TRBV4-1 or TRAV17.

In some embodiments, the antigen binding domain of the sixth domain binds to a TAA.

In some embodiments, the second multispecific molecule comprises a sixth domain comprising a cytokine.

In some embodiments, the antigen binding domain of the fourth domain binds to TRAV1-2, TRBV6-2, TRBV4-1 or TRAV17.

In some embodiments, the antigen binding domain of the fourth domain binds to a TAA.

Also described herein is a pharmaceutical composition comprising the composition described herein.

Also described herein is a method of expanding a T cell comprising: contacting a population of cells comprising the T cell with the composition described herein, wherein the T cell binds to CD1b or CD1c presented antigens.

Also described herein is a method of activating a T cell comprising: contacting a population of cells comprising the T cell with the composition described herein, wherein the T cell binds to CD1b or CD1c presented antigens.

Also described herein is a method of expanding a T cell comprising: contacting the T cell with a composition comprising a molecule comprising a first domain that binds to a first target molecule, wherein the T cell binds to CD1b or CD1c presented antigens, wherein the first target molecule is TRAV1-2, TRBV6-2, TRBV4-1 or TRAV17, thereby expanding the T cell.

Also described herein is a method of activating a T cell comprising: contacting the T cell with a composition comprising a molecule comprising a first domain that binds to a first target molecule, wherein the T cell binds to CD1b or CD1c presented antigens, wherein the first target molecule is TRAV1-2, TRBV6-2, TRBV4-1 or TRAV17, thereby activating the T cell.

In some embodiments, the T cell is a GEM T cell or a LDN5-like T cell.

In some embodiments, the method is performed in vitro or ex vivo.

In some embodiments, the method is performed in vivo.

In some embodiments, the method comprises expanding the T cell at least 2-fold, wherein the T cell binds to Cd1b or Cd1c presented antigens.

In some embodiments, the method comprises selectively expanding the T cell, wherein the T cell binds to Cd1b or Cd1c presented antigens.

In some embodiments, the method comprises expanding the T cell, thereby producing an expanded cell population, wherein at least 1% of the cells in the expanded population of cells are the T cells, wherein the T cell binds to Cd1b or Cd1c presented antigens.

In some embodiments, the method comprises expanding the T cell, thereby producing an expanded cell population, wherein at most 20% of the cells in the expanded population of cells are the T cells, wherein the T cell binds to Cd1b or Cd1c presented antigens.

In some embodiments, the method specifically activates the T cell of the population of cells, wherein the T cell binds to Cd1b or Cd1c presented antigens.

In some embodiments, the T cell expresses TRAV1-2, TRBV6-2, TRBV4-1 or TRAV17.

In some embodiments, the T cell is CD4+/CD8−.

In some embodiments, the T cell is CD4−/CD8+.

In some embodiments, the T cell is CD4−/CD8−.

In some embodiments, the T cell is CD69+.

In some embodiments, the method comprises expanding the T cell at least 5-fold or at least 10-fold, wherein the T cell binds to CD1b or CD1c presented antigens.

In some embodiments, the method comprises activating the T cell, wherein the T cell binds to CD1b or CD1c presented antigens, thereby producing an activated cell population, wherein the activated cell population produces: (i) a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising the T cell, wherein the T cell binds to Cd1b or Cd1c presented antigens, with an anti-CD3 antibody at the same concentration, an anti-TRBV antibody that does not bind TRBV4-1 or TRBV6-2 at the same concentration, or an anti-TRAV antibody that does not bind TRAV1-2 or TRAV17 at the same concentration according to a cytokine release assay, or (ii) a lower level of IL-4 IL-5, IL-10, IL-21, or IL-22 compared to the level of the IL-4 IL-5, IL-10, IL-21, or IL-22 produced by a cell population produced by contacting the population of cells comprising the T cell, wherein the T cell binds to Cd1b or Cd1c presented antigens, with an anti-CD3 antibody at the same concentration, an anti-TRBV antibody that does not bind TRBV4-1 or TRBV6-2 at the same concentration, or an anti-TRAV antibody that does not bind TRAV1-2 or TRAV17 at the same concentration according to a cytokine release assay.

In some embodiments, the activated cell population produces a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising the T cell, wherein the T cell binds to Cd1b or Cd1c presented antigens, with an anti-CD3 antibody at the same concentration.

In some embodiments, the activated cell population produces a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising T cell, wherein the T cell binds to Cd1b or Cd1c presented antigens, with an anti-TRAV antibody that does not bind TRAV1-2 at the same concentration.

In some embodiments, the activated cell population produces a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising T cell, wherein the T cell binds to Cd1b or Cd1c presented antigens, with an anti-TRBV antibody that does not bind TRBV6-2 at the same concentration.

In some embodiments, the activated cell population produces a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising T cell, wherein the T cell binds to Cd1b or Cd1c presented antigens, with an anti-TRBV antibody that does not bind TRBV4-1 at the same concentration.

In some embodiments, the activated cell population produces a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising T cell, wherein the T cell binds to Cd1b or Cd1c presented antigens, with an anti-TRAV antibody that does not bind TRAV17 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising the T cell, wherein the T cell binds to Cd1b or Cd1c presented antigens, with an anti-CD3 antibody at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRAV antibody that does not bind TRAV1-2 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRBV antibody that does not bind TRBV6-2 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRBV antibody that does not bind TRBV4-1 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRAV antibody that does not bind TRAV17 at the same concentration.

Also described herein is a method of treating a disease or condition in a subject in need thereof comprising administering to the subject a therapeutically effective amount of the composition described herein or pharmaceutical composition described herein.

Also described herein is a method of treating a disease or condition in a subject in need thereof comprising administering to the subject a therapeutically effective amount of a composition comprising a molecule comprising a first domain that binds to a first target molecule, wherein the first target molecule is TRAV1-2, TRBV6-2, TRBV4-1 or TRAV17.

In some embodiments, the disease or condition is cancer.

In some embodiments, the cancer is a solid tumor, a hematological cancer, a metastatic cancer, a soft tissue tumor, or any combination thereof.

In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and any combination thereof.

In some embodiments, the cancer is the hematological cancer, and wherein the hematological cancer is selected from the group consisting of Hodgkin's lymphoma. Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof.

In some embodiments, the method further comprising administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the composition sequentially, simultaneously, or concurrently.

In some embodiments, the disease or condition is an infection.

In some embodiments, the infection is selected from the group consisting of a S. parathyphi infection, a Bacteroidetes infection, a Proteobacteria infection, a M. tuberculosis infection, a respiratory virus infection, a SARS-CoV-2 infection, yellow fever, influenza, a HIV infection, a Cytomegalovirus infection, and an Epstein-Barr Virus infection.

In some embodiments, the multispecific molecule comprises a first polypeptide and a second polypeptide, wherein the first polypeptide and the second polypeptide are non-contiguous, wherein the first polypeptide comprises each of the following linked together: (i) a first portion of a dimerization module; and (ii)(A) a third domain comprising an antigen binding domain, wherein the third domain comprises a single chain variable fragment (scFv), or a single domain antibody (sdAb), or (B) a first portion of the third domain, wherein the first portion of the third domain comprises a heavy chain variable domain (VH), wherein when the first polypeptide comprises the first portion of the third domain, the multispecific molecule further comprises a third polypeptide comprising a second portion of the third domain, wherein the second portion of the third domain comprises a light chain variable domain (VL), wherein the third polypeptide is non-contiguous with the first polypeptide and the second polypeptide; and wherein the second polypeptide comprises each of the following linked together: (iii) a second portion of the dimerization module; (iv) the second domain, wherein the second domain comprises a cytokine; (v)(A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: (i) a first portion of a dimerization module; (ii)(A) the second domain comprising an antigen binding domain, wherein the second domain comprises a single chain variable fragment (scFv), or a single domain antibody (sdAb), or (B) a first portion of the second domain, wherein the first portion of the second domain comprises a heavy chain variable domain (VH), wherein when the first polypeptide comprises the first portion of the second domain, the multispecific molecule further comprises a third polypeptide comprising a second portion of the second domain, wherein the second portion of the second domain comprises a light chain variable domain (VL), wherein the third polypeptide is non-contiguous with the first polypeptide and the second polypeptide; and wherein the second polypeptide comprises each of the following linked together: (iii) a second portion of the dimerization module; and (iv)(A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: (i) a first portion of a dimerization module; (ii) (A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and b.(iii) (A) the second domain, wherein the second domain comprises the cytokine or a functional fragment or variant thereof, or a single chain variable fragment (scFv), or a single domain antibody (sdAb), or i.(B) a first portion of the second domain, wherein the second domain comprises the antigen binding domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide; wherein the second polypeptide comprises each of the following linked together: (i) a second portion of the dimerization module; (ii)(A) an additional first domain, wherein the additional first domain comprises a single chain variable fragment (scFv), or a single domain antibody (sdAb), or ii.(B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and c.(iii) (A) an additional second domain, wherein the second domain comprises the cytokine or a functional fragment or variant thereof, or a single chain variable fragment (scFv), or a single domain antibody (sdAb), or (B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide, and/or the fifth polypeptide, the sixth polypeptide is non-contiguous with the third polypeptide, the fourth polypeptide, and the fifth polypeptide.

In some embodiments, the first portion of the dimerization module comprises a first immunoglobulin constant region (Fc region) and the second portion of the dimerization module comprises a second Fc region.

In some embodiments, the first Fc region, the second Fc region, or a combination thereof is selected from the group consisting of IgG1, IgG2, IgG3, IgG4, IgAQ1, IgA2, IgJ, IgM, IgD, IgE, and any fragment thereof.

In some embodiments, the first Fc region, the second Fc region, or a combination thereof comprises an IgGI constant region comprising one or more amino acid substitutions selected from the group consisting of N297A, K322A, L234A and L235A.

In some embodiments, the first Fc region, the second Fc region, or a combination thereof comprises a sequence having at least 75% sequence identity to the sequence SEQ ID NO: 1040, SEQ ID NO: 1042, SEQ ID NO: 1204, SEQ ID NO: 1205, SEQ ID NO: 1041, SEQ ID NO: 1206, or SEQ ID NO: 1207.

In some embodiments, the first domain that binds to TRAV1-2, TRBV6-2, or TRBV4-1, a second domain that binds to TRAV1-2, TRBV6-2, TRBV4-1 or TRAV17, or any combination thereof comprises a Fab, a F(ab′)2, an Fv, a single chain Fv (scFv), a single domain antibody, a diabody (dAb), a camelid antibody, and a combination thereof.

In some embodiments, the first domain that binds to TRAV1-2, TRBV6-2, TRBV4-1 or TRAV17, a second domain that binds to TRAV1-2, TRBV6-2, TRBV4-1 or TRAV17, or any combination thereof comprises a Fab or an scFv.

In some embodiments, the first domain that binds to (i) TRAV1-2 comprises a VH comprising complementarity determining regions (VHCDR1-3) of SEQ ID NOs: 1197-1199, respectively, and a VL comprising complementarity determining regions (VLCDR1-3) of SEQ ID NOs: 1200-1202, respectively; or (ii) TRBV4-1 comprises: (a) a VH comprising complementarity determining regions (VH-CDR1-3) that comprise the sequences of: SEQ ID NOs: 1208, 1209, 1210, respectively; SEQ ID NOs: 1211, 1212, 1210, respectively; or SEQ ID NOs: 1213, 1209, 1210, respectively; (b) a VL comprising complementarity determining regions (VLCDR1-3) that comprise the sequences of: SEQ ID NOs: 1214, 1215, 1216, respectively; or SEQ ID NOs: 1217, 1215, 1216, respectively; or (c) any combination thereof; or (iii) TRBV6-2 comprises: (a) a VH comprising complementarity determining regions (VHCDR1-3) that comprise the sequences of: SEQ ID NOs: 1003, 1004, 1005, respectively; SEQ ID NOs: 1045, 1004, 1005, respectively; SEQ ID NOs: 1015, 1016, 1005, respectively; SEQ ID NOs: 1164, 1165, 1005, respectively; SEQ ID NOs: 1164, 1170, 1005, respectively; SEQ ID NOs: 1174, 1175, 1005, respectively: SEQ ID NOs: 1164, 1178, 1005, respectively; SEQ ID NOs: 1174, 1171, 1005, respectively: SEQ ID NOs: 1174, 1314, 1005, respectively; SEQ ID NOs: 1174, 1192, 1005, respectively; SEQ ID NOs: 1164, 1175, 1005, respectively; SEQ ID NOs: 1229, 1165, 1005, respectively; SEQ ID NOs: 1234, 1165, 1005, respectively; or SEQ ID NOs: 1164, 1165, 1167, respectively; (b) a VL comprising complementarity determining regions (VLCDR1-3) that comprise the sequences of: SEQ ID NOs: 1006, 1007, 1008, respectively; SEQ ID NOs: 1166, 1167, 1168, respectively; SEQ ID NOs: 1171, 1167, 1008, respectively; SEQ ID NOs: 1313, 1167, 1008, respectively; SEQ ID NOs: 1176, 1167, 1008, respectively: SEQ ID NOs: 1179, 1167, 1008, respectively; SEQ ID NOs: 1172, 1167, 1008, respectively; SEQ ID NOs: 1182, 1167, 1008, respectively; SEQ ID NOs: 1186, 1167, 1008, respectively: SEQ ID NOs: 1176, 1188, 1008, respectively: SEQ ID NOs: 1176, 1167, 1190, respectively: SEQ ID NOs: 1177, 1299, 1008, respectively; SEQ ID NOs: 1171, 1299, 1008, respectively; or SEQ ID NOs: 1170, 1171, 1008, respectively; or (c) any combination thereof.

In some embodiments, the first domain that binds to (i) TRAV1-2 comprises a VH comprising a sequence having at least 75% sequence identity to the sequence of SEQ ID NO: 1195, and a VL comprising a sequence having at least 75% sequence identity to the sequence of SEQ ID NO: 11%; or (ii) TRBV4-1 comprises: (a) a VH comprising a sequence having at least 75% sequence identity to the sequence of SEQ ID NO: 1219, SEQ ID NO: 1220, SEQ ID NO: 1221, SEQ ID NO: 1222, or SEQ ID NO: 1223; (b) a VL comprising a sequence having at least 75% sequence identity to the sequence of SEQ ID NO: 1218, SEQ ID NO: 1224, SEQ ID NO: 1225, SEQ ID NO: 1226, SEQ ID NO: 1227; or (c) any combination thereof; or (iii) TRBV6-2 comprises: (a) a VH comprising a sequence having at least 75% sequence identity to the sequence of: SEQ ID NO: 1001; SEQ ID NO: 1009; SEQ ID NO: 1025; SEQ ID NO: 1027: SEQ ID NO: 1029; SEQ ID NO: 1030: SEQ ID NO: 1033; SEQ ID NO: 1036; SEQ ID NO: 1038; SEQ ID NO: 1047; SEQ ID NO: 1050; SEQ ID NO: 1056; SEQ ID NO: 1059; SEQ ID NO: 1063; SEQ ID NO: 1065: SEQ ID NO: 1067; SEQ ID NO: 1070; SEQ ID NO: 1072; SEQ ID NO: 1078: SEQ ID NO: 1080; SEQ ID NO: 1083; SEQ ID NO: 1085: SEQ ID NO: 1088; SEQ ID NO: 1090. SEQ ID NO: 1092; SEQ ID NO: 1094; SEQ ID NO: 1096: SEQ ID NO: 1097; SEQ ID NO: 1100; SEQ ID NO: 1104; SEQ ID NO: 1106; SEQ ID NO: 1108: SEQ ID NO: 1111; SEQ ID NO: 1113; SEQ ID NO: 1115; SEQ ID NO: 1117: SEQ ID NO: 1120; SEQ ID NO: 1122; SEQ ID NO: 1123; SEQ ID NO: 1125; SEQ ID NO: 1168; SEQ ID NO: 1172; SEQ ID NO: 1173; SEQ ID NO: 1177; SEQ ID NO: 1180: SEQ ID NO: 1183; SEQ ID NO: 1185; SEQ ID NO: 1193; SEQ ID NO: 1194; or SEQ ID NO: 1317: (b) a VL comprising a sequence having at least 75% sequence identity to the sequence of: SEQ ID NO: 1002; SEQ ID NO: 1010; SEQ ID NO: 1011; SEQ ID NO: 1024; SEQ ID NO: 1029: SEQ ID NO: 1032; SEQ ID NO: 1035; SEQ ID NO: 1049; SEQ ID NO: 1055; SEQ ID NO: 1058: SEQ ID NO: 1062; SEQ ID NO: 1082; SEQ ID NO: 1087; SEQ ID NO: 1096: SEQ ID NO: 1102; SEQ ID NO: 1110; SEQ ID NO: 1148; SEQ ID NO: 1169; SEQ ID NO: 1173: SEQ ID NO: 1184; SEQ ID NO: 1187; SEQ ID NO: 1189; SEQ ID NO: 1191: SEQ ID NO: 1300; SEQ ID NO: 1303; or (c) any combination thereof.

In some embodiments, the first domain binds to TRAV1-2.

In some embodiments, the composition further comprises a second multispecific molecule the second multispecific molecule comprises a fourth domain that binds to TRBV6-2 TRBV4-1, or TRAV17.

In some embodiments, the first domain binds to TRBV6-2.

In some embodiments, the composition further comprises a second multispecific molecule the second multispecific molecule comprises a fourth domain that binds to TRBV1-2, TRBV4-1, or TRAV17.

In some embodiments, the first domain binds to TRBV4-1.

In some embodiments, the composition further comprises a second multispecific molecule the second multispecific molecule comprises a fourth domain that binds to TRAV1-2, TRBV4-1, or TRAV17.

In some embodiments, the first domain binds to TRAV17.

In some embodiments, the composition further comprises a second multispecific molecule, wherein the second multispecific molecule comprises a fourth domain that binds to TRAV1-2, TRBV6-2, or TRBV4-1.

In some embodiments, the second multispecific molecule comprises a fifth domain, and wherein the fifth domain comprises (i) a cytokine or a functional fragment or variant thereof, or (ii) an antigen binding domain.

In some embodiments, the fifth domain comprises a cytokine.

In some embodiments, the fifth domain comprises an antigen binding domain.

In some embodiments, the second multispecific molecule comprises a sixth domain comprising an antigen binding domain.

In some embodiments, the antigen binding domain of the sixth domain binds to TRAV1-2, TRBV6-2, TRBV4-1 or TRAV17.

In some embodiments, the antigen binding domain of the sixth domain binds to a TAA.

In some embodiments, the second multispecific molecule comprises a sixth domain comprising a cytokine.

In some embodiments, the antigen binding domain of the fourth domain binds to TRAV1-2, TRBV6-2, TRBV4-1 or TRAV17.

In some embodiments, the antigen binding domain of the fourth domain binds to a TAA.

Also described herein is a pharmaceutical composition comprising the composition described herein.

Also described herein is a method of expanding a T cell comprising: contacting a population of cells comprising the T cell with the described herein, wherein the T cell binds to CD1b or CD1c presented antigens.

Also described herein is a method of activating a T cell comprising: contacting a population of cells comprising the T cell with the composition described herein, wherein the T cell binds to CD1b or CD1c presented antigens.

Also described herein is a method of expanding a T cell comprising: contacting the T cell with a composition comprising a molecule comprising a first domain that binds to a first target molecule, wherein the T cell binds to CD1b or CD1c presented antigens, wherein the first target molecule is TRAV1-2, TRBV6-2, TRBV4-1 or TRAV17, thereby expanding the T cell.

Also described herein is a method of activating a T cell comprising: contacting the T cell with a composition comprising a molecule comprising a first domain that binds to a first target molecule, wherein the T cell binds to CD1b or CD1c presented antigens, wherein the first target molecule is TRAV1-2, TRBV6-2, TRBV4-1 or TRAV17, thereby activating the T cell.

In some embodiments, the T cell is a GEM T cell or a LDN5-like T cell.

In some embodiments, the method is performed in vitro or ex vivo.

In some embodiments, the method is performed in vivo.

In some embodiments, the method comprises expanding the T cell at least 2-fold, wherein the T cell binds to Cd1b or Cd1c presented antigens.

In some embodiments, the method comprises selectively expanding the T cell, wherein the T cell binds to Cd1b or Cd1c presented antigens.

In some embodiments, the method comprises expanding the T cell, thereby producing an expanded cell population, wherein at least 1% of the cells in the expanded population of cells are the T cells, wherein the T cell binds to Cd1b or Cd1c presented antigens.

In some embodiments, the method comprises expanding the T cell, thereby producing an expanded cell population, wherein at most 20% of the cells in the expanded population of cells are the T cells, wherein the T cell binds to Cd1b or Cd1c presented antigens.

In some embodiments, the method specifically activates the T cell of the population of cells, wherein the T cell binds to Cd1b or Cd1c presented antigens.

In some embodiments, the T cell expresses TRAV1-2, TRBV6-2, TRBV4-1 or TRAV17.

In some embodiments, the T cell is CD4+/CD8−. In some embodiments, the T cell is CD4−/CD8+. In some embodiments, the T cell is CD4−/CD8−. In some embodiments, the T cell is CD69+.

In some embodiments, the method comprises expanding the T cell at least 5-fold or at least 10-fold, wherein the T cell binds to CD1b or CD1c presented antigens.

In some embodiments, the method comprises activating the T cell, wherein the T cell binds to CD1b or CD1c presented antigens, thereby producing an activated cell population, wherein the activated cell population produces: (i) a higher level of IL-1RA, IL-6, IL-13, IL-17, IL-25, CCL17, IFN-7, or TNF compared to the level of the IL-1RA, IL-6, IL-13, IL-17, IL-25, CCL17, IFN-7, or TNF produced by a cell population produced by contacting the population of cells comprising the T cell, wherein the T cell binds to Cd1b or Cd1c presented antigens, with an anti-CD3 antibody at the same concentration, an anti-TRBV antibody that does not bind TRBV4-1 or TRBV6-2 at the same concentration, or an anti-TRAV antibody that does not bind TRAV1-2 or TRAV17 at the same concentration according to a cytokine release assay, or (ii) a lower level of IL-4 IL-5, IL-10, IL-21, or IL-22 compared to the level of the IL-4 IL-5, IL-10, IL-21, or IL-22 produced by a cell population produced by contacting the population of cells comprising the T cell, wherein the T cell binds to Cd1b or Cd1c presented antigens, with an anti-CD3 antibody at the same concentration, an anti-TRBV antibody that does not bind TRBV4-1 or TRBV6-2 at the same concentration, or an anti-TRAV antibody that does not bind TRAV1-2 or TRAV17 at the same concentration according to a cytokine release assay.

In some embodiments, the activated cell population produces a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising the T cell, wherein the T cell binds to Cd1b or Cd1c presented antigens, with an anti-CD3 antibody at the same concentration.

In some embodiments, the activated cell population produces a higher level of IL-1RA, IL-6, IL-13, IL-17, IL-25, CCL17, IFN-7, or TNF compared to the level of the IL-1RA, IL-6, IL-13, IL-17, IL-25, CCL17, IFN-γ, or TNF produced by a cell population produced by contacting the population of cells comprising T cell, wherein the T cell binds to Cd1b or Cd1c presented antigens, with an anti-TRAV antibody that does not bind TRAV1-2 at the same concentration.

In some embodiments, the activated cell population produces a higher level of IL-1RA, IL-6, IL-13, IL-17, IL-25, CCL17, IFN-7, or TNF compared to the level of the IL-1RA, IL-6, IL-13, IL-17, IL-25, CCL17, IFN-7, or TNF produced by a cell population produced by contacting the population of cells comprising T cell, wherein the T cell binds to Cd1b or Cd1c presented antigens, with an anti-TRBV antibody that does not bind TRBV6-2 at the same concentration.

In some embodiments, the activated cell population produces a higher level of IL-1RA, IL-6, IL-13, IL-17, IL-25, CCL17, IFN-γ, or TNF compared to the level of the IL-1RA, IL-6, IL-13, IL-17, IL-25, CCL17, IFN-γ, or TNF produced by a cell population produced by contacting the population of cells comprising T cell, wherein the T cell binds to Cd1b or Cd1c presented antigens, with an anti-TRBV antibody that does not bind TRBV4-1 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising the T cell, wherein the T cell binds to Cd1b or Cd1c presented antigens, with an anti-CD3 antibody at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRAV antibody that does not bind TRAV1-2 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRBV antibody that does not bind TRBV6-2 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRBV antibody that does not bind TRBV4-1 at the same concentration.

In some embodiments, the activated cell population produces a lower level of IL-4 IL-5, IL-10, IL-21, or IL-22 compared to the level of the IL-4 IL-5, IL-10, IL-21, or IL-22 produced by a cell population produced by contacting the population of cells comprising T cell, wherein the T cell binds to Cd1b or Cd1c presented antigens, with an anti-TRAV antibody that does not bind TRAV17 at the same concentration.

Also described herein is a method of treating a disease or condition in a subject in need thereof comprising administering to the subject a therapeutically effective amount of the composition described herein or pharmaceutical composition described herein.

Also described herein is a method of treating a disease or condition in a subject in need thereof comprising administering to the subject a therapeutically effective amount of a composition comprising a molecule comprising a first domain that binds to a first target molecule, wherein the first target molecule is TRAV1-2, TRBV6-2, TRBV4-1 or TRAV17.

In some embodiments, the disease or condition is cancer.

In some embodiments, the cancer is a solid tumor, a hematological cancer, a metastatic cancer, a soft tissue tumor, or any combination thereof.

In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and any combination thereof.

In some embodiments the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof.

In some embodiments, the method further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the composition sequentially, simultaneously, or concurrently.

In some embodiments, the disease or condition is an infection.

In some embodiments, the infection is selected from the group consisting of a S. parathyphi infection, a Bacteroidetes infection, a Proteobacteria infection, a M. tuberculosis infection, a respiratory virus infection, a SARS-CoV-2 infection, yellow fever, influenza, a HIV infection, a Cytomegalovirus infection, and an Epstein-Barr Virus infection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-ID are schematic representations of exemplary structures of antibody molecules that bind to TCR. FIG. 1A depicts an antibody molecule comprising an anti-TCR fab, an Fc dimer, and a cytokine. The C-terminus of the cytokine is fused to the N-terminus of one Fc chain while the C-terminus of the heavy chain of anti-TCR fab is fused to the N-terminus of the other Fc chain. FIG. 1B depicts an antibody molecule comprising an anti-TRAV1-2 fab, an Fc dimer, and IL-2. The C-terminus of the cytokine is fused to the N-terminus of one Fc chain while the C-terminus of the heavy chain of anti-TRAV1-2 fab is fused to the N-terminus of the other Fc chain. FIG. 1C depicts an antibody molecule comprising an anti-TRBV6 fab, an Fc dimer, and IL-2. The C-terminus of the cytokine is fused to the N-terminus of one Fc chain while the C-terminus of the heavy chain of anti-TRBV6 fab is fused to the N-terminus of the other Fc chain. FIG. 1D depicts an antibody molecule comprising an anti-TRBV20-1 fab, an Fc dimer, and IL-2. The C-terminus of the cytokine is fused to the N-terminus of one Fc chain while the C-terminus of the heavy chain of anti-TRBV20-1 fab is fused to the N-terminus of the other Fc chain.

FIGS. 2A-2D are schematic representations of exemplary structures of multispecific antibody molecules (e.g., bispecific antibodies) that bind to TAA and TCR. FIG. 2A depicts a bispecific antibody molecule comprising an anti-TAA fab, an anti-TCR scFv, an Fc dimer, and a cytokine. The C-terminus of the heavy chain of the anti-TAA fab is fused to the N-terminus of one Fc chain. The anti-TCR scFv is fused to the N-terminus of the cytokine and the C-terminus of the cytokine is fused to the N-terminus of the other Fc chain. FIG. 2B depicts a bispecific antibody molecule comprising an anti-TAA fab, an anti-TRAV1-2 scFv, an Fc dimer, and IL-2. The C-terminus of the heavy chain of the anti-TAA fab is fused to the N-terminus of one Fc chain. The anti-TRAV1-2 scFv is fused to the N-terminus of IL-2 and the C-terminus of IL-2 is fused to the N-terminus of the other Fc chain. FIG. 2C depicts a bispecific antibody molecule comprising an anti-TAA fab, an anti-TRBV6 scFv, an Fc dimer, and IL-2. The C-terminus of the heavy chain of the anti-TAA fab is fused to the N-terminus of one Fc chain. The anti-TRBV6 scFv is fused to the N-terminus of IL-2 and the C-terminus of IL-2 is fused to the N-terminus of the other Fc chain. FIG. 2D depicts a bispecific antibody molecule comprising an anti-TAA fab, an anti-TRBV20-1 scFv, an Fc dimer, and IL-2. The C-terminus of the heavy chain of the anti-TAA fab is fused to the N-terminus of one Fc chain. The anti-TRBV20-1 scFv is fused to the N-terminus of IL-2 and the C-terminus of IL-2 is fused to the N-terminus of the other Fc chain.

FIGS. 3A-3D are schematic representations of exemplary structures of multispecific antibody molecules (e.g., bispecific antibodies) that bind to TAA and TCR. FIG. 3A depicts a bispecific antibody molecule comprising an anti-TAA fab, an anti-TCR scFv, and an Fc dimer. The C-terminus of the heavy chain of the anti-TAA fab is fused to the N-terminus of one Fc chain. The anti-TCR scFv is fused to the N-terminus of the other Fc chain. FIG. 3B depicts a bispecific antibody molecule comprising an anti-TAA fab, an anti-TRAV1-2 scFv, and an Fc dimer. The C-terminus of the heavy chain of the anti-TAA fab is fused to the N-terminus of one Fc chain. The anti-TRAV1-2 scFv is fused to the N-terminus of the other Fc chain. FIG. 3C depicts a bispecific antibody molecule comprising an anti-TAA fab, an anti-TRBV6 scFv, and an Fc dimer. The C-terminus of the heavy chain of the anti-TAA fab is fused to the N-terminus of one Fc chain. The anti-TRBV20-1 scFv is fused to the N-terminus of the other Fc chain.

FIGS. 4A-G are schematic representations of exemplary structures of multispecific antibody molecules (e.g., bispecific, or trispecific antibodies) that bind to TAA and TCR. FIG. 4A depicts a multispecific antibody molecule comprising two anti-TAA fab, two anti-TCR scFv, and an Fc dimer. The anti-TCR scFv is fused to the N-terminus of the anti-TAA fab and the C-terminus of the anti-TAA fab is fused to the N terminus of one Fc chain. The (same or different) anti-TCR scFv is fused to the N-terminus of the anti-TAA fab and the C-terminus of the anti-TAA fab is fused to the N terminus of the other Fc chain. FIG. 4B depicts a multispecific antibody molecule comprising two anti-TAA fab, two anti-TRAV1-2 scFv, and an Fc dimer. The anti-TRAV1-2 scFv is fused to the N-terminus of the anti-TAA fab and the C-terminus of the anti-TAA fab is fused to the N terminus of one Fc chain. The anti-TRAV1-2 scFv is fused to the N-terminus of the anti-TAA fab and the C-terminus of the anti-TAA fab is fused to the N terminus of the other Fc chain. FIG. 4C depicts a multispecific antibody molecule comprising two anti-TAA fab, two anti-TRBV6 scFv, and an Fc dimer. The anti-TRBV6 scFv is fused to the N-terminus of the anti-TAA fab and the C-terminus of the anti-TAA fab is fused to the N terminus of one Fc chain. The anti-TRBV6 scFv is fused to the N-terminus of the anti-TAA fab and the C-terminus of the anti-TAA fab is fused to the N terminus of the other Fc chain. FIG. 4D depicts a multispecific antibody molecule comprising two anti-TAA fab, two anti-TRBV20-1 scFv, and an Fc dimer. The anti-TRBV20-1 scFv is fused to the N-terminus of the anti-TAA fab and the C-terminus of the anti-TAA fab is fused to the N terminus of one Fc chain. The anti-TRBV20-1 scFv is fused to the N-terminus of the anti-TAA fab and the C-terminus of the anti-TAA fab is fused to the N terminus of the other Fc chain. FIG. 4E depicts a multispecific antibody molecule comprising two anti-TAA fab, an anti-TRBV20-1 scFv, an anti-TRAV1-2 scFv, and an Fc dimer. The anti-TRBV20-1 scFv is fused to the N-terminus of the anti-TAA fab and the C-terminus of the anti-TAA fab is fused to the N terminus of one Fc chain. The anti-TRAV1-2 scFv is fused to the N-terminus of the anti-TAA fab and the C-terminus of the anti-TAA fab is fused to the N terminus of the other Fc chain. FIG. 4F depicts a multispecific antibody molecule comprising two anti-TAA fab, an anti-TRBV6 scFv, an anti-TRAV1-2 scFv, and an Fc dimer. The anti-TRBV6 scFv is fused to the N-terminus of the anti-TAA fab and the C-terminus of the anti-TAA fab is fused to the N terminus of one Fc chain. The anti-TRAV1-2 scFv is fused to the N-terminus of the anti-TAA fab and the C-terminus of the anti-TAA fab is fused to the N terminus of the other Fc chain. FIG. 4G depicts a multispecific antibody molecule comprising two anti-TAA fab, an anti-TRBV20-1 scFv, an anti-TRAV1-2 scFv, and an Fc dimer. The anti-TRBV20-1 scFv is fused to the N-terminus of the anti-TAA fab and the C-terminus of the anti-TAA fab is fused to the N terminus of one Fc chain. The anti-TRAV1-2 scFv is fused to the N-terminus of the anti-TAA fab and the C-terminus of the anti-TAA fab is fused to the N terminus of the other Fc chain.

FIG. 5 shows an exemplary embodiment of multispecific molecules as described herein that comprises an TRBV25 or TRAV10 binding domain and a cytokine.

FIG. 6 shows an exemplary embodiment of multispecific molecules as described herein that comprises an TRBV25 or TRAV10 binding domain, a cytokine, and a tumor associate antigen (TAA) binding domain.

FIG. 7 shows an exemplary embodiment of multispecific molecules as described herein that comprises an TRBV25 or TRAV10 binding domain and a TAA binding domain.

FIG. 8 shows an exemplary embodiment of multispecific molecules as described herein that comprises two TRBV25 or TRAV10 binding domains and two TAA binding domains.

FIG. 9A depicts an exemplary embodiment of a multifunctional molecule containing a single molecule of an exemplary cytokine, interleukin-2 (IL-2), linked to a first dimerization domain and an exemplary antibody molecule that binds to a T cell receptor beta variable region or a T cell receptor alpha variable region (“anti-TCRvβ or anti-TCRvα”), linked to a second dimerization domain.

FIG. 9B depicts an exemplary embodiment of a multifunctional molecule containing an exemplary antibody molecule that binds to a tumor associated antigen (TAA), linked to a first dimerization domain and an exemplary anti-TCRvβ or anti-TCRvα antibody molecule, linked to an exemplary cytokine, IL-2, linked to a second dimerization domain.

FIG. 9C depicts an exemplary embodiment of a multispecific molecule containing an exemplary antibody molecule that binds to a TAA, linked to a first dimerization domain and an exemplary anti-TCRvβ or anti-TCRvα antibody molecule linked to a second dimerization domain.

FIG. 9D depicts an exemplary embodiment of a multispecific molecule containing an exemplary antibody molecule that binds to a TAA, an exemplary anti-TCRvβ or anti-TCRvα antibody molecule, linked to a first dimerization domain and an exemplary antibody molecule that binds to a TAA, an exemplary anti-TCRvβ or anti-TCRvα antibody molecule, linked to a second dimerization domain.

### DETAILED DESCRIPTION

### Part I

Provided herein are compositions and methods for expanding and/or activating a specific T cell using a multispecific molecule, wherein the specific T cell comprises a MAIT cell expressing TRAV1-2, TRBV6, and TRBV20, wherein the MAIT cell is targeted by the multispecific molecule, thereby providing treatments for treat a variety of diseases.

### Definition

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. The use of the words “a” or “an” when used in conjunction with the term “comprising” herein may mean “one,” but it is also consistent with the meaning of “one or more,” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods. As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

“Antibody molecule” as used herein refers to a protein, e.g., an immunoglobulin chain or fragment thereof, comprising at least one immunoglobulin variable domain structure and/or sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs). In some embodiments, the antibody molecule is an antibody mimetic. In some embodiments, the antibody molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” as used herein refers to a humanized antibody molecule, human antibody molecule or an antibody molecule having at least 95% sequence identity with a non-murine germline framework region, e.g., FR1, FR2, FR3 and/or FR4. In some embodiments, the human-like antibody molecule comprises a framework region having at least 95% sequence identity to a human germline framework region, e.g., a FR1, FR2, FR3 and/or FR4 of a human germline framework region. In some embodiments, the human-like antibody molecule is a recombinant antibody. In some embodiments, the human-like antibody molecule is a humanized antibody molecule. In some embodiments, the human-like antibody molecule is human antibody molecule. In some embodiments, the human-like antibody molecule is a phage display or a yeast display antibody molecule. In some embodiments, the human-like antibody molecule is a chimeric antibody molecule. In some embodiments, the human-like antibody molecule is a CDR grafted antibody molecule.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule. e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196.901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, an “immune cell” refers to any of various cells that function in the immune system. e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated.” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated polynucleotide (ribonucleic acid (RNA), deoxyribonucleic acid (DNA)), or polypeptide is free of the genes/nucleic acids or sequences/amino acids that flank it in its naturally-occurring state.

The compositions and methods of the present disclosure encompass polypeptides and nucleic acids having the sequences specified, or sequences substantially identical or similar thereto, e.g., sequences at least 80%, 85%, 90° %, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant. In some embodiments, a TCRαV variant can bind to TCRα and form a TCR α:β complex.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using aNWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the disclosure. BLAST protein searches can be performed with the XBLAST program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present disclosure may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “mutation” refers to an alteration in the nucleotide sequence of the genome of an organism, virus, or extrachromosomal DNA. In some embodiments, the mutation may be a large-scale mutation, such as amplifications (or gene duplications) or repetitions of a chromosomal segment, deletions of large chromosomal regions, chromosomal rearrangements (e.g., chromosomal translocations, chromosomal inversions, non-homologous chromosomal crossover, and interstitial deletions), and loss of heterozygosity. In some embodiments, the mutation may be a small-scale mutation, such as insertions, deletions, and substitution mutations. As used herein, the term “substitution mutation” refers to the transition that exchange a single nucleotide for another.

### Human T Cell Receptor (TCR) Complex

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRαV chains can be further classified into subfamilies (TRAV1-10, 12-14, 16-27, 29, 30, 34-36 an 38-41). The TCRβV chains can be further classified into 30 subfamilies (TRBV1-30). Despite their high structural and functional homology, the amino acid sequence homology in the TRAV or TRBV genes is very low. Nevertheless, TCRs formed between alpha and beta chains of highly diverse sequences show a remarkable structural and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G, and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor alpha variable chain” or “TCRαV,” or “TRAV,” refers to an extracellular region of the T cell receptor alpha chain which can comprise a portion of the antigen recognition domain of the T cell receptor. The term TCRαV includes isoforms, mammalian, e.g., human TCRαV, species homologs of human and analogs comprising at least one common epitope with TCRαV. Human TCRαV comprises a gene family comprising subfamilies including, but not limited to: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, or a TCRα V41 subfamily, as well as family members of said subfamilies, and variants thereof (e.g., a structural or functional variant thereof).

In some embodiments, the TCRα V1 subfamily comprises: TCRαV1-1 or TCRαV1-2, or a variant thereof.

In some embodiments, the TCRα V8 subfamily comprises: TCRαV8-1, TCRαV8-2, TCRαV8-3, TCRαV8-4, or TCRαV8-6, or a variant thereof.

In some embodiments, the TCRα V9 subfamily comprises: TCRαV9-1 or TCRαV9-2, or a variant thereof.

In some embodiments, the TCRα V12 subfamily comprises: TCRαV12-1, TCRαV12-2, or TCRαV12-3, or a variant thereof.

In some embodiments, the TCRα V13 subfamily comprises: TCRαV13-1 or TCRαV13-2, or a variant thereof.

In some embodiments, the TCRα V14 subfamily comprises: TCRαV14/DV4, or a variant thereof.

In some embodiments, the TCRα V23 subfamily comprises: TCRαV23/DV6, or a variant thereof.

In some embodiments, the TCRα V26 subfamily comprises: TCRαV26-1 or TCRαV26-2, or a variant thereof.

In some embodiments, the TCRα V29 subfamily comprises: TCRαV29/DV5, or a variant thereof.

In some embodiments, the TCRα V36 subfamily comprises: TCRαV236/DV7, or a variant thereof.

In some embodiments, the TCRα V38 subfamily comprises: TCRαV38-1 or TCRαV38-2/DV8, or a variant thereof.

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCR V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, as well as family members of said subfamilies, and variants thereof (e.g., a structural or functional variant thereof).

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR alpha or beta gene undergoes gene arrangement to generate diversity.

The TCR V alpha or beta repertoire varies between individuals and populations because of, e.g., 7 frequently occurring inactivating polymorphisms in functional gene segments and a large insertion/deletion-related polymorphism encompassing 2 V alpha or beta gene segments.

Provided herein are, inter alia, antibody molecules and fragments thereof, that bind, e.g., specifically bind, to a human TCR alpha V chain (TCRαV), e.g., a TCRαV gene family (also referred to as a group), e.g., a TCRαV subfamily (also referred to as a subgroup), e.g., as described herein, or to a human TCR beta V chain (TCRβV), e.g., a TCRβV gene family (also referred to as a group), e.g., a TCRβV subfamily (also referred to as a subgroup), e.g., as described herein. TCR alpha or beta V families and subfamilies are known in the art, e.g., as described in Yassai et al., (2009) Immunogenetics 61(7)pp: 493-502; Wei S, and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRAV, TCRVA, TRAV, TCRαV, TCRVα or TRαV are used interchangeably herein and refer to a TCR alpha V chain, e.g., as described herein.

The terms TCRBV, TCRVB, TRBV, TCRβV, TCRVβ or TRPV are used interchangeably herein and refer to a TCR beta V chain, e.g., as described herein.

Exemplary amino acid sequences for TCRαV or TCRVβ subfamily members can be found on the ImMunoGeneTics Information System website: http://www.imgt.org/, or in a similar resource.

### Anti-TCRαV Antibodies

Current bispecific constructs designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vomhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRαV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRαV antibody molecules as described herein, which despite having low sequence similarity (e.g., low sequence identity among the different antibody molecules that recognize different TCRαV subfamilies), recognize a structurally conserved, yet sequence-wise variable, region, e.g., domain, on the TCRαV protein and have a similar function (e.g., activation of T cells and a similar cytokine profile as described herein). Thus, the anti-TCRαV antibody molecules as described herein share a structure-function relationship.

In some embodiments, the anti-TCRαV antibody molecules as described herein do not recognize, e.g., bind to, an interface of a TCRβ V:TCRalpha complex. In some embodiments, the anti-TCRαV antibody molecules as described herein do not recognize, e.g., bind to, a constant region of a TCRβV protein. In some embodiments, the anti-TCRαV antibody molecules as described herein do not recognize, e.g., bind to, one or more (e.g., all) of a complementarity determining region (e.g., CDR1, CDR2 and/or CDR3) of a TCRβ V protein.

Provided herein are, inter alia, antibody molecules directed to the variable chain of the alpha subunit of TCR (TCRαV) which bind and, e.g., activate a subset of T cells. The anti-TCRαV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRαV antibodies as described herein have a cytokine profile. e.g., as described herein, which differs from a cytokine profile of a T cell engager that binds to a receptor or molecule other than a TCRαV region (“a non-TCRαV-binding T cell engager”). In some embodiments, the non-TCRαV-binding T cell engager comprises an antibody that binds to a CD3 molecule (e.g., CD3 epsilon (CD3e) molecule); or a TCR alpha (TCRα) molecule. In some embodiments, the non-TCRαV-binding T cell engager is an OKT3 antibody or an SP34-2 antibody.

In some embodiments, the anti-TCRαV antibodies as described herein result in expansion of TCRαV+ T cells, e.g., a subset of T cells known as MAITs. Accordingly, provided herein are methods of making said anti-TCRαV antibody molecules and uses thereof. Also described herein are multispecific molecules, e.g., bispecific molecules comprising said anti-TCRαV antibody molecules. In some embodiments, compositions comprising anti-TCRαV antibody molecules of the present disclosure, can be used, e.g., to: (1) activate and redirect T cells to promote tumor cell lysis for cancer immuno-therapy; and/or (2) expand TCRαV+ T cells. In some embodiments, compositions comprising anti-TCRαV antibody molecules as described herein limit the harmful side-effects of CRS and/or NT, e.g., CRS and/or NT associated with anti-CD3e targeting.

In some embodiments, the anti-TCRαV antibody molecule is a full antibody or fragment thereof (e.g., a Fab, F(ab′)₂, Fv, single domain antibody, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgGI or IgG2 (e.g., human IgGI, or IgG2). In some embodiments, the heavy chain constant region is human IgG1. In some embodiments, the Fc region comprises a Fc region variant, e.g., as described herein.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgGI.

The various TCRαV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference.

In some embodiments, the anti-TCRαV antibody molecule is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule is isolated or recombinant.

In some embodiments, the anti-TCRαV antibody molecule comprises a heavy chain constant region for an IgG4, e.g., a human IgG4. In still another embodiment, the anti-TCRαV antibody molecule includes a heavy chain constant region for an IgG1, e.g., a human IgG1. In some embodiments, the heavy chain constant region comprises an amino sequence set forth in Table 1, or a sequence substantially identical (e.g., at least 80%, 85%, 90%, 92%, 95%, 97%, 98%, 99% or higher identical) thereto.

In some embodiments, the anti-TCRαV antibody molecule includes a kappa light chain constant region, e.g., a human kappa light chain constant region. In some embodiments, the light chain constant region comprises an amino sequence set forth in Table 1, or a sequence substantially identical (e.g., at least 80%, 85%, 90%, 92%, 95%, 97%, 98%, 99% or higher identical) thereto.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule, is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule, is isolated or recombinant.

In some embodiments, the anti-TCRαV antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule comprises a light chain variable domain comprising: (a) a framework region 1 (FRI) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRαV antibody molecule is a full antibody or fragment thereof (e.g., a Fab, F(ab′)₂, Fv, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgGI or IgG2 (e.g., human IgGI, or IgG2). In some embodiments, the heavy chain constant region is human IgGI.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

### Anti-TCRβV Antibodies

Described herein are molecules targeting the TCRβV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRαV antibody molecules as described herein, which despite having low sequence similarity (e.g., low sequence identity among the different antibody molecules that recognize different TCRβV subfamilies), recognize a structurally conserved, yet sequence-wise variable, region, e.g., domain, on the TCRβV protein (as denoted by the circled area in FIG. 25A) and have a similar function (e.g., activation of T cells and a similar cytokine profile as described herein). Thus, the anti-TCRβV antibody molecules as described herein share a structure-function relationship.

Without wishing to be bound by theory, in some embodiments, the anti-TCRαV antibody molecules as described herein bind to an outward facing epitope of a TCRαV protein when it is in a complex with a TCRalpha protein, e.g., as denoted by the circled area in FIG. 25A. In some embodiments, the anti-TCRβV antibody molecules as described herein recognize (e.g., bind to), a domain (e.g., an epitope) on the TCRβV protein that is: (1) structurally conserved among different TCRβV subfamilies; and (2) has minimal sequence identity among the different TCRβV subfamilies.

In some embodiments, the anti-TCRβV antibody molecules as described herein do not recognize, e.g., bind to, an interface of a TCRβV:TCRalpha complex. In some embodiments, the anti-TCRβV antibody molecules as described herein do not recognize, e.g., bind to, a constant region of a TCRβV protein. An exemplary antibody that binds to a constant region of a TCRBV region is JOVI.1 as de-scribed in Viney et al., (Hybridoma. 1992 December; 11(6):701-13). In some embodiments, the anti-TCRβV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the beta subunit of TCR (TCRβV) which bind and, e.g., activate a subset of T cells. The anti-TCRβV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRβV antibodies as described herein have a cytokine profile, e.g., as described herein, which differs from a cytokine profile of a T cell engager that binds to a receptor or molecule other than a TCRBV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in expansion of TCRβV+ T cells, e.g., a subset of T cells known as MAIT. Accordingly, provided herein are methods of making said anti-TCRβV antibody molecules and uses thereof. Also described herein are multispecific molecules, e.g., bispecific molecules comprising said anti-TCRβV antibody molecules. In some embodiments, compositions comprising anti-TCRβV antibody molecules of the present disclosure, can be used, e.g., to: (1) activate and redirect T cells to promote tumor cell lysis for cancer immuno-therapy; and/or (2) expand TCRβV+ T cells. In some embodiments, compositions comprising anti-TCRβV antibody molecules as described herein limit the harmful side-effects of CRS and/or NT, e.g., CRS and/or NT associated with anti-CD3e targeting.

In some embodiments, the anti-TCRβV antibody molecule binds to one or more of TRBV6-1, TRBV6-2, TRBV6-3, TRBV6-4, TRBV6-5, TRBV6-6, TRBV6-8, TRBV6-9, and TRBV20-1. In some embodiments, the anti-TCRBV antibody molecule binds to one or more of TRBV6-1, TRBV6-2, TRBV6-3, TRBV6-4, TRBV6-5, TRBV6-6, TRBV6-8 and TRBV6-9. In some embodiments, the anti-TCRβV antibody molecule is an anti-TRBV6-1, anti-TRBV6-2, anti-TRBV6-3, anti-TRBV6-4, anti-TRBV6-5, anti-TRBV6-6, anti-TRBV6-8, anti-TRBV6-9, or anti-TRBV20-1.

In some embodiments, the anti-TCRβV antibody molecule binds specifically to TRBV6-1, TRBV6-2, TRBV6-3, TRBV6-4, TRBV6-5, TRBV6-6, TRBV6-8, TRBV6-9TRBV10-1, TRBV10-2, or TRBV10-3. In some embodiments, the anti-TCRβV antibody molecule binds specifically to TRBV6-1. In some embodiments, the anti-TCRβV antibody molecule binds specifically to TRBV6-2. In some embodiments, the anti-TCRβV antibody molecule binds specifically to TRBV6-3. In some embodiments, the anti-TCRβV antibody molecule binds specifically to TRBV6-4. In some embodiments, the anti-TCRβV antibody molecule binds specifically to TRBV6-5. In some embodiments, the anti-TCRβV antibody molecule binds specifically to TRBV6-6. In some embodiments, the anti-TCRβV antibody molecule binds specifically to TRBV6-8. In some embodiments, the anti-TCRβV antibody molecule binds specifically to TRBV6-9. In some embodiments, the anti-TCRβV antibody molecule binds specifically to TRBV20-1

### Antibody Molecules

In some embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In some embodiments, an antibody molecule is a monospecific antibody molecule and binds a single epitope. E.g., a monospecific antibody molecule having a plurality of immunoglobulin variable domain sequences, each of which binds the same epitope.

In some embodiments, an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or a tetraspecific antibody molecule.

In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv or a Fab, or fragment thereof, have binding specificity for a first epitope and a scFv or a Fab, or fragment thereof, have binding specificity for a second epitope.

In some embodiments, an antibody molecule comprises a diabody, and a single-chain molecule, as well as an antigen-binding fragment of an antibody (e.g., Fab, F(ab′)2, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci, USA 85:5879-5883): (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the disclosure, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the disclosure.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally. e.g., Protein Sequence and Structure Analysis of Antibody Variable Domains. In: Antibody Engineering Lab Manual (Ed.: Duebel, S, and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner et al. U.S. Pat. No. 5,223,409; Kang et al. International Publication No. WO 92/18619: Dower et al. International Publication No. WO 91/17271; Winter et al. International Publication WO 92/20791: Markland et al. International Publication No. WO 92/15679; Breitling et al. International Publication WO 93/01288; McCafferty et al. International Publication No. WO 92/01047; Garrard et al. International Publication No. WO 92/0990; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci, USA 81:6851-6855; Bruggeman et al. 1993 Year Immunol 7:33-40; Tuaillon et al. 1993 PNAS 90:3720-3724; Bruggeman et a. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the disclosure. Antibody molecules generated in a non-human organism, e.g., a rat or mouse, and then modified, e.g., in the variable framework or constant region, to decrease antigenicity in a human are within the disclosure.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al. Cancer Immunol. Immunother., 32:180-190 (1990)) and also because of potential allergic reactions (see. e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526: Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present disclosure (UK Patent Application GB 2188638A, filed on Mar. 26, 1987; Winter U.S. Pat. No. 5,225,539), the contents of which is expressly incorporated by reference.

Also within the scope of the disclosure are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher. D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the disclosure are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### CDR-Grafted Scaffolds

In some embodiments, the antibody molecule is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Multispecific Molecules

In some embodiments, the multispecific molecules as described herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgGI For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender. e.g., a human serum albumin or an antibody molecule to human serum albumin.

In some embodiments, Fc contains exemplary Fc modifications listed in Table 8.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 4, with additional optional stabilizing Fc cysteine mutations.

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgGI hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgGI antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CHI and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CHI and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US/20150211001, US20140072581A1, US20160039947A1, and US20150368352.

### Mucosal-Associated Invariant T (MAIT) Cells

As used herein the term “MAIT” cells, also known as mucosal-associated invariant T (MAIT) cells, are innate-like T cells defined by their expression of a semi-invariant αβ T cell receptor (TCR) which recognizes biosynthetic derivatives of riboflavin synthesis presented on the restriction molecule major histocompatibility complex (MHC)-related protein-1 (MR1).

MAIT cells contrast with conventional T cells which have highly variable TCRs, capable of targeting a vast array of peptide epitopes produced by viruses, bacteria, and malignant cells. Conventional T cells therefore have exquisite specificity for individual peptides, and individual clones may undergo massive expansion, to provide T cell memory. However, at the first encounter with a pathogen the frequency of any individual peptide-specific T cell will be very low. In contrast, the MAIT cell TCR provides an innate capacity to respond to a specific set of ligands without the need for expansion.

MAIT cells have an intrinsic effector-memory phenotype, usually CD45RA⁻CD45RO⁺ CD95^{Hi}CD62^{Lo}-CD44^{Hi}, with capacity for rapid secretion of several pro-inflammatory cytokines.

In humans, MAIT cells display a restricted αβ T cell receptor (TCR), in which the TCRVα chain comprises a canonical Vα7.2-Jα33 (from now on the IMGT denomination will be used: TRAV1-2-TRAJ33) rearrangement, paired with a limited number of TCRβ chains: TRBV20-1, TRBV6-1, TRBV6-4, TRBV6-5, and TRBV-13, and less frequent usage of the non-canonical TRAV1-2-TRAJ12/20 TCR rearrangement. They can be identified by staining for TRAV1-2 and either CD161, or IL-18Rα, in the TCR7S CD4-CD3⁺ compartment.

MAIT cells are remarkably abundant in human tissues, typically comprising 1-4% of all T cells in peripheral blood and up to 10% of airway T cells and 20-40% of liver T cells. As each TCR recognizes the same ligand, early in an immune response, MAIT cells will markedly exceed the numbers of conventional antigen-specific T cells responding to cognate antigens. The MR1-MAIT cell axis is strikingly conserved across 150 million years of mammalian evolution, with ˜90% sequence homology for MR1 between mouse and human, implying a strong evolutionary pressure maintaining the MAIT cell repertoire.

MAIT cells can be activated in response to TCR ligation by riboflavin intermediates presented on MR1, under co-stimulatory signals from specific cytokines or toll-like receptors (TLR). Activated cells expand substantially inducing a rapid innate-like immune response and effector functions including anti-microbial cytotoxic products, inflammatory chemokines, and cytokines.

Unlike conventional T cells which recognize peptide antigen presented by MHC molecules, MAIT cells are restricted by MR1, a non-polymorphic, β2-microglobulin-associated antigen-presenting molecule, widely expressed in multiple tissues. Unlike class 1 and class 2 MHC, MR1 does not constitutively present self-ligands. Generally, MR1 molecules reside in the endoplasmic reticulum (ER) in an incompletely folded ligand-receptive conformation, free of β2 microglobulin. Riboflavin metabolites are transported to the ER, bind MR1 via formation of a Schiff base, followed by a complete folding and association with 02-microglobulin. The temary complex then traffics through the ER and the Golgi to the cell membrane. Although recycling of the MR1 molecule can occur, most MR1 molecules are degraded and reintemalized intracellularly, which contributes to MR1's rapid presentation of extracellular riboflavin antigens and MAIT cells' rapid activation. Moreover, NF-κB signaling is necessary for MR1 signal transduction. Either bone marrow-derived antigen-presenting cell (APC) such as dendritic cells, monocytes, macrophages, B cells, or non-bone marrow-derived epithelial cells, can activate MAIT cells via MR1.

As with conventional T cells, MR1-TCR signaling alone is insufficient to fully activate MAIT cells which also require co-stimulation by CD28, TLR agonists, bacterial products, or cytokines. Such cytokines include interleukin (IL)-7, tumor necrosis factor (TNF), type-I interferons (IFNs), IL-10 and/or IL-23. MAIT cells express several cytokine receptors including IL-7R, IL-12R, IL-15R, IL-18R, and IL-23R. IL-7 enhances MAIT cell responses to bacteria and promotes cytotoxicity. IL-12 and IL-18 potentiate MR1-dependent bacterial MAIT cell activation. Agonists of the pathogen recognition receptors TLR1, TLR2 and TLR6 in humans, and TLR3, TLR4, TLR6/2, and TLR9 in mice promote MAIT cell activation mainly in an indirect way through the activation of APCs via enhancement of MR1 presentation, stimulation of cytotoxic molecules and inflammatory cytokines or up-regulation of co-stimulatory ligands. In addition, inducible T cell co-stimulator, highly expressed by MAIT cells is also essential for optimal activation and maintenance of retinoic acid-related orphan receptor γt (RORγt) expression.

In the absence of TCR-mediated antigen recognition, MAIT cells can also be partially activated by cytokines, such as IL-7, IL-12, IL-15, IL-18, and type-I IFNs, broadening the potential range of pathogens to which MAIT cells can respond to include viruses.

TCR-mediated and -independent activation work synergistically in optimal MAIT cell activation. Upon stimulation, there is increased expression of activation markers CD69 and CD25, degranulation marker CD107a, production of cytotoxic substances such as perform and granzyme B, secretion of pro-inflammatory cytokines including IFN-γ, TNF, IL-17, and colony stimulating factor 2 (CSF2/GM-CSF) and release of chemokines such as XCL1, CCL3, CCL4, and CXCL16. MAIT cells exert antimicrobial activity not only by direct recognition and killing of infected cells, but also indirectly, for example by recruiting neutrophils, increasing bactericidal activity of phagocytes, promoting the production of IFN-γ from DCs, and promoting monocyte to DC differentiation. (https://www.frontiersin.org/articles/i0.3389/fimmu.2020.01014/full).

MAIT cells typically express a TRAV1-2⁺ semi-invariant TCRα that enables recognition of bacterial, mycobacterial, and fungal riboflavin metabolites presented by MR1.

### Composition Comprising a Multispecific Molecules

As used herein, a “multifunctional” or a “multispecific” molecule refers to molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multispecific molecule is a multispecific antibody molecule, e.g., a bispecific antibody molecule. In some embodiments, the multispecific molecule includes an anti-TRAV or anti-TRBV antibody molecule as described herein.

In one aspect, the disclosure provides a composition comprising a multispecific molecule, wherein the multispecific molecule comprises (a) a first domain that binds to a first target molecule, wherein the first target molecule is T cell receptor alpha variable 1-2 (TRAV1-2), T cell receptor beta variable 20 (TRBV20), T cell receptor beta variable 6 (TRBV6), or both TRBV20 and TRBV6, and (b) a second domain that binds to a second target molecule, wherein the second domain comprises (i) a cytokine or a functional fragment or variant thereof, or (ii) an antigen binding domain.

### MAIT Cell Associated Markers

In some embodiments, the composition further comprises a T cell. In some embodiments, the T cell comprises a mucosal-associated invariant T (MAIT) cell.

In some embodiments, the MAIT cell can be CD8(+), CD4(+), CD4(−) CD8(−) double-negative (DN), or CD4(+) CD8(+) double-positive (DP). In some embodiments, the MAIT cell can be CD8(+). In some embodiments, the MAIT cell can be CD4(+). In some embodiments, the MAIT cell can be DN. In some embodiments, the MAIT cell can be DP.

In some embodiments, the MAIT cell can express MAIT cell associated markers, for example, any one of TRAV1-2, TRAJ33, TRAJ12, TRAJ20, TRAV36, TRAJ34, TRAJ37 TRBV6, TRBV20, TRBV28, TRBJ2, or any combination thereof. In some embodiments, the MAIT cell expresses TRAV1-2, TRBV6, TRBV20, or both TRBV6 and TRBV20, or any combination thereof.

The MAIT cell expresses several cytokine receptors comprising IL-7R, IL-12R, IL-15R, IL-18R, or IL-23R. Activation and expansion of a MAIT cell can be triggered by a cytokine. In some embodiments, the cytokine is any one selected from the group consisting of an interleukin-2 (IL-2) molecule or functional fragment or variant thereof, an interleukin-7 (IL-7) molecule or functional fragment or variant thereof, an interleukin-12 (IL-12) molecule or functional fragment or variant thereof, an interleukin-15 (IL-15) molecule or functional fragment or variant thereof, an interleukin-18 (IL-18) molecule or functional fragment or variant thereof, an interleukin-21 (IL-21) molecule or functional fragment or variant thereof, an interferon gamma molecule or functional fragment or variant thereof, and any combination thereof.

### TRAV1-2

In some embodiments, TRAV1-2 comprises a VH comprising heavy chain complementarity determining regions (HC CDR1-3) comprising an amino acid sequence with at least 80%, 85%, 90%, 91%, 92%, 93%, 95%, 96%, 97%, 98%, 99%, or 100% sequence identity to the sequence of SEQ ID NOs: 197-199, respectively, and/or a VL comprising a light chain complementarity determining regions (LC CDR1-3) comprising an amino acid sequence with at least 80%, 85%, 90%, 91%, 92%, 93%, 95%, 96%, 97%, 98%, 99%, or 100% sequence identity to the sequence of SEQ ID NOs: 200-202, respectively.

### TRBV20

In some embodiments, TRBV20 comprises TRBV20-1. In some embodiments, TRBV20-1 comprises TRBV20-1*01, TRBV20-1*02, TRBV20-1*03, TRBV20-1*04, TRBV20-1*05, TRBV20-1*06, or TRBV20-1*07. In some embodiments, TRBV20-1 comprises TRBV20-1*01. In some embodiments, TRBV20-1 comprises TRBV20-1*02. In some embodiments, TRBV20-1 comprises TRBV20-1*03. In some embodiments, TRBV20-1 comprises TRBV20-1*04. In some embodiments, TRBV20-1 comprises TRBV20-1*05. In some embodiments. TRBV20-1 comprises TRBV20-1*06. In some embodiments, TRBV20-1 comprises TRBV20-1*07.

In some embodiments, TRBV20-1 comprises a VH comprising heavy chain complementarity determining regions (HC CDR1-3) comprising an amino acid sequence with at least 80%, 85%, 90%, 91%, 92%, 93%, 95%, 96%, 97%, 98%, 99%, or 100% sequence identity to the sequence of SEQ ID NOs: 339-341, respectively, and a VL comprising light chain complementarity determining regions (LC CDR1-3) comprising an amino acid sequence with at least 80%, 85%, 90%, 91%, 92%, 93%, 95%, 96%, 97%, 98%, 99%, or 100% sequence identity to the sequence of SEQ ID NOs: 342-344, respectively.

### TRBV6

In some embodiments, TRBV6 comprises TRBV6-1, TRBV6-2, TRBV6-3, TRBV6-4, TRBV6-5, TRBV6-6, TRBV6-8, or TRBV6-9. In some embodiments, TRBV6 comprises TRBV6-1. In some embodiments, TRBV6-1 comprises TRBV6-1*01. In some embodiments, TRBV6 comprises TRBV6-2. In some embodiments, TRBV6-2 comprises TRBV6-2*01. In some embodiments, TRBV6 comprises TRBV6-3. In some embodiments, TRBV6-3 comprises TRBV6-3*01. In some embodiments, TRBV6 comprises TRBV6-4. In some embodiments, TRBV6-4 comprises TRBV6-4*01, or TRBV6-4*02. In some embodiments, TRBV6-4 comprises TRBV6-4*01. In some embodiments, TRBV6-4 comprises TRBV6-4*02. In some embodiments, TRBV6 comprises TRBV6-5. In some embodiments, TRBV6-5 comprises TRBV6-5*01. In some embodiments, TRBV6 comprises TRBV6-6. In some embodiments, TRBV6-6 comprises TRBV6-6*01, TRBV6-6*02, TRBV6-6*03, TRBV6-6*04, or TRBV6-6*05. In some embodiments, TRBV6-6 comprises TRBV6-6*01. In some embodiments. TRBV6-6 comprises TRBV6-6*02. In some embodiments, TRBV6-6 comprises TRBV6-6*03. In some embodiments, TRBV6-6 comprises TRBV6-6*04. In some embodiments, TRBV6-6 comprises TRBV6-6*05. In some embodiments, TRBV6 comprises TRBV6-8. In some embodiments, TRBV6-8 comprises TRBV6-8*01. In some embodiments, TRBV6 comprises TRBV6-9. In some embodiments, TRBV6-9 comprises TRBV6-9*01.

In some embodiments, TRBV6 comprises a VH comprising a HC CDR1 comprising an amino acid sequence with at least 80%, 85%, 90%, 91%, 92%, 93%, 95%, 96%, 97%, 98%, 99%, or 100% sequence identity to the sequence of SEQ ID NO: 3, 45, 15, 164, 174, 229, 234, 314, 317, or 319, a HC CDR2 comprising an amino acid sequence with at least 80%, 85%, 90%, 91%, 92%, 93%, 95%, 96%, 97%, 98%, 99%, or 100% sequence identity to the sequence of SEQ ID NO: 4, 16, 165, 170, 175, 178, 181, 365, 192, 315, or 318, a HC CDR3 comprising an amino acid sequence with at least 80%, 85%, 90%, 91%, 92%, 93%, 95%, 96%, 97%, 98%, 99%, or 100% sequence identity to the sequence of SEQ ID NO: 5, or 316, or any combination thereof; a VL comprising a LC CDR1 comprising an amino acid sequence with at least 80%, 85%, 90%, 91%, 92%, 93%, 95%, 96%, 97%, 98%, 99%, or 100% sequence identity to the sequence of SEQ ID NO: 6, 166, 171, 364, 176, 179, 182, 186, 320, or 323, a LC CDR2 comprising an amino acid sequence with at least 80%, 85%, 90%, 91%, 92%, 93%, 95%, 96%, 97%, 98%, 99%, or 100% sequence identity to the sequence of SEQ ID NO: 7, 167, 188, 299, or 321, a LC CDR3 comprising an amino acid sequence with at least 80%, 85%, 90%, 91%, 92%, 93%, 95%, 96%, 97%, 98%, 99%, or 100% sequence identity to the sequence of SEQ ID NO: 8, 190, or 322, or any combination thereof; or any combination thereof.

### Cytokine

As used herein, a “cytokine molecule” or a “cytokine polypeptide” as interchangeably used herein, refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

In some embodiments, the cytokine molecule is an interleukin or a variant, e.g., a functional variant thereof. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Mutant cytokine molecules useful as effector moieties in the multispecific molecules are also considered herein. Mutant cytokines can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the multispecific molecule of the disclosure binds to a cytokine receptor with a dissociation constant (KD) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific molecule binds to a cytokine receptor with a KD that is at least 2, 3, 4, 5, 6, 7, 8, 9, or 10 times greater than that for a corresponding multispecific molecule comprising two or more effector moieties. In another embodiment, the multispecific molecule binds to a cytokine receptor with a dissociation constant KD that is about 10 times greater than that for a corresponding the multispecific molecule comprising two or more cytokines.

In some embodiments, the cytokine molecule as used herein includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor. The cytokine molecule can be a monomer or a dimer. In some embodiments, the cytokine molecule can further include a cytokine receptor dimerizing domain.

In some embodiments, the cytokine comprises IL-2 or a functional fragment or variant thereof. In some embodiments, the IL-2 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 268.

In some embodiments, the cytokine comprises IL-7 or a functional fragment or variant thereof. In some embodiments, the IL-7 or the functional fragment or variant thereof comprises a sequence of SEQ ID NO: 273.

In some embodiments, the cytokine comprises IL-12 or a functional fragment or variant thereof. In some embodiments, the IL-12 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 276.

In some embodiments, the cytokine comprises IL-15 or a functional fragment or variant thereof. In some embodiments, the IL-15 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 297.

In some embodiments, the cytokine comprises IL-18 or a functional fragment or variant thereof. In some embodiments, the IL-18 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 285.

In some embodiments, the cytokine comprises IL-21 or a functional fragment or variant thereof. In some embodiments, the IL-21 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 286.

In some embodiments, the cytokine comprises interferon gamma or a functional fragment or variant thereof. In some embodiments, the interferon gamma or the functional fragment or variant thereof comprises a sequence having at least 60%, 65%, 70%, 75%, 80%, 85%, 90%, 95%, 99%. 99.5%. 99.9%, or 100% sequence identity to the sequence of

(SEQ ID NO: 313)
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

### Multispecific Molecule

In some embodiments, the multispecific molecule is bound to a T cell, e.g., a CD8(+) T cells, CD4(+) T cell, and a T regulatory cell. In some embodiments, the multispecific molecule is bound to a MAIT cell. In some embodiments, the multispecific molecule is bound to a MAIT cell by targeting one or more of the MAIT cell associated markers disclosed herein, e.g., the multispecific molecule targets TRAV1-2, TRBV6, TRBV20, both TRBV6 and TRBV20, or any combination thereof that is expressed on the MAIT cell surface.

In some embodiments, the multispecific molecule is an agonist of TRAV1-2, TRBV20, TRBV6, or both TRBV20 and TRBV6. In some embodiments, the multispecific molecule is an antagonist of TRAV1-2, TRBV20, TRBV6, or both TRBV20 and TRBV6.

### First Domain of the Multispecific Molecule

In some embodiments, the first domain of the multispecific molecule binds to a MAIT cell. In some embodiments, the first domain of the multispecific molecule binds to one or more of a MAIT cell associated marker. In some embodiments, the first domain of the multispecific molecule binds to one or more of the MAIT cell associated marker disclosed herein, e.g., TRAV1-2, TRBV20, TRBV6, or both TRBV20 and TRBV6. In some embodiments, the first domain of the multispecific molecule can bind to TRAV1-2. In some embodiments, the first domain of the multispecific molecule can bind to TRBV20. In some embodiments, the first domain of the multispecific molecule can bind to TRBV6. In some embodiments, the first domain of the multispecific molecule can bind to both TRBV20 and TRBV6.

### Second Domain of the Multispecific Molecule

In some embodiments, the second domain of the multispecific molecule comprises a cytokine or a functional fragment or variant thereof, or an antigen binding domain.

In some embodiments, the second domain of the multispecific molecule comprises a cytokine or a functional fragment or variant thereof disclosed herein, e.g, IL-2, IL-7, IL-12, IL-15, IL-18, IL-21, an interferon gamma, a functional fragment or variant thereof, and any combination thereof.

In some embodiments, the second domain of the multispecific molecule comprises an antigen binding domain. In some embodiments, the antigen comprises a MAIT cell associated marker. In some embodiments, the antigen comprises a TAA domain.

In some embodiments, the second domain of the multispecific molecule binds to one or more of a MAIT cell associated marker. In some embodiments, the second domain of the multispecific molecule binds to one or more of the MAIT cell associated marker disclosed herein, e.g., TRAV1-2, TRBV20, TRBV6, both TRBV20 and TRBV6, or any combination thereof. In some embodiments, the second domain of the multispecific molecule can bind to TRAV1-2. In some embodiments, the second domain of the multispecific molecule can bind to TRBV20. In some embodiments, the second domain of the multispecific molecule can bind to TRBV6. In some embodiments, the second domain of the multispecific molecule can bind to both TRBV20 and TRBV6.

In some embodiments, the antigen binding domain of the second domain can bind to a tumor associated antigen (TAA), for example a TAA disclosed herein.

### Third Domain

In some embodiments, the third domain of the multispecific molecule comprises a cytokine or a functional fragment or variant thereof, or an antigen binding domain.

In some embodiments, the third domain of the multispecific molecule comprises a cytokine or a functional fragment or variant thereof disclosed herein, e.g, IL-2, IL-7, IL-12, IL-15, IL-18, IL-21, an interferon gamma, a functional fragment or variant thereof, and any combination thereof.

In some embodiments, the third domain of the multispecific molecule comprises an antigen binding domain. In some embodiments, the antigen comprises a MAIT cell associated marker. In some embodiments, the antigen comprises a TAA domain.

In some embodiments, the third domain of the multispecific molecule binds to one or more of a MAIT cell associated marker. In some embodiments, the third domain of the multispecific molecule binds to one or more of the MAIT cell associated marker disclosed herein. e.g., TRAV1-2, TRBV20, TRBV6, both TRBV20 and TRBV6, or any combination thereof. In some embodiments, the third domain of the multispecific molecule can bind to TRAV1-2. In some embodiments, the third domain of the multispecific molecule can bind to TRBV20. In some embodiments, the third domain of the multispecific molecule can bind to TRBV6. In some embodiments, the third domain of the multispecific molecule can bind to both TRBV20 and TRBV6.

In some embodiments, the antigen binding domain of the third domain can bind to a tumor associated antigen (TAA), for example a TAA disclosed herein.

### Tumor Associated Antigen (TAA)

In some embodiments, the TAA is a tumor antigen, a stromal antigen, or a hematological antigen. In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met. Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

### Configuration of the Multispecific Molecule

In some embodiments, the multispecific molecule comprises a single TRAV1-2-, TRBV20-, TRBV6-, or TRBV20- and TRBV6-binding moiety. In some embodiments, the multispecific molecule comprises a single TRAV1-2-binding moiety. In some embodiments, the multispecific molecule comprises a single TRBV20-binding moiety. In some embodiments, the multispecific molecule comprises a single TRBV6-binding moiety. In some embodiments, the multispecific molecule comprises a single TRBV20- and TRBV6-binding moiety. In some embodiments, the multispecific molecule comprises two or more TRAV1-2-, TRBV20-, TRBV6-, or TRBV20- and TRBV6-binding moieties. In some embodiments, the multispecific molecule comprises TRAV1-2- and TRBV6-binding moieties. In some embodiments, the multispecific molecule comprises TRAV1-2- and TRBV20-binding moieties. In some embodiments, the multispecific molecule comprises TRAV1-2-, TRBV20-, and TRBV6-binding moieties. In some embodiments, the multispecific molecule comprises TRBV20- and TRBV6-binding moieties. In some embodiments, the multispecific molecule comprises TRBV20- and TRBV20- and TRBV6-binding moieties. In some embodiments, the multispecific molecule comprises TRBV6- and TRBV20- and TRBV6-binding moieties.

In some embodiments, the first domain and the antigen binding domain of the second domain binds to a MAIT cell associated marker. In some embodiments, the first domain and the antigen binding domain of the second domain binds to a MAIT cell associated marker, for example, any one of TRAV1-2, TRBV20, TRBV6, or both TRBV20 and TRBV6.

In some embodiments, the first domain binds to TRAV1-2 and the antigen binding domain of the second domain binds to a MAIT cell associated marker, for example, any one of TRAV1-2, TRBV20, TRBV6, or both TRBV20 and TRBV6. In some embodiments, the first domain binds to TRBV20 and the antigen binding domain of the second domain binds to a MAIT cell associated marker, for example, any one of TRAV1-2, TRBV20, TRBV6, or both TRBV20 and TRBV6. In some embodiments, the first domain binds to TRBV6 and the antigen binding domain of the second domain binds to a MAIT cell associated marker, for example, any one of TRAV1-2, TRBV20, TRBV6, or both TRBV20 and TRBV6. In some embodiments, the first domain binds to TRBV6 and TRBV20 and the antigen binding domain of the second domain binds to MAIT cell associated markers, for example, any one of TRAV1-2, TRBV20, TRBV6, or both TRBV20 and TRBV6.

In some embodiments, the first domain and the antigen binding domain of the third domain binds to a MAIT cell associated marker. In some embodiments, the first domain and the antigen binding domain of the third domain binds to a MAIT cell associated marker, for example, any one of TRAV1-2, TRBV20, TRBV6, or both TRBV20 and TRBV6.

In some embodiments, the first domain binds to TRAV1-2 and the antigen binding domain of the third domain binds to a MAIT cell associated marker, for example, any one of TRAV1-2, TRBV20, TRBV6, or both TRBV20 and TRBV6. In some embodiments, the first domain binds to TRBV20 and the antigen binding domain of the third domain binds to a MAIT cell associated marker, for example, any one of TRAV1-2, TRBV20, TRBV6, or both TRBV20 and TRBV6. In some embodiments, the first domain binds to TRBV6 and the antigen binding domain of the third domain binds to a MAIT cell associated marker, for example, any one of TRAV1-2, TRBV20, TRBV6, or both TRBV20 and TRBV6. In some embodiments, the first domain binds to TRBV6 and TRBV20 and the antigen binding domain of the third domain binds to MAIT cell associated markers, for example, any one of TRAV1-2, TRBV20, TRBV6, or both TRBV20 and TRBV6.

In some embodiments, the first domain and the antigen binding domain of the second and the third domain binds to a MAIT cell associated marker. In some embodiments, the first domain and the antigen binding domain of the second and the third domain binds to a MAIT cell associated marker, for example, any one of TRAV1-2, TRBV20, TRBV6, or both TRBV20 and TRBV6.

In some embodiments, the first domain binds to TRAV1-2 and the antigen binding domain of the second domain and third domain binds to a MAIT cell associated marker, for example, any one of TRAV1-2, TRBV20, TRBV6, or both TRBV20 and TRBV6. In some embodiments, the first domain binds to TRBV20 and the antigen binding domain of the second domain and third domain binds to a MAIT cell associated marker, for example, any one of TRAV1-2, TRBV20, TRBV6, or both TRBV20 and TRBV6. In some embodiments, the first domain binds to TRBV6 and the antigen binding domain of the second domain and third domain binds to a MAIT cell associated marker, for example, any one of TRAV1-2, TRBV20, TRBV6, or both TRBV20 and TRBV6. In some embodiments, the first domain binds to TRBV6 and TRBV20 and the antigen binding domain of the second domain and third domain binds to MAIT cell associated markers, for example, any one of TRAV1-2, TRBV20, TRBV6, or both TRBV20 and TRBV6.

In some embodiments, the first domain binds to TRAV1-2 and the antigen binding domain of the second domain binds to TRAV1-2. In some embodiments, the first domain binds to TRBV20 and the antigen binding domain of the second domain binds to TRBV20. In some embodiments, the first domain binds to TRBV6 and the antigen binding domain of the second domain binds to TRBV6. In some embodiments, the first domain binds to both TRBV20 and TRBV6 and the antigen binding domain of the second domain binds to both TRBV20 and TRBV6.

In some embodiments, the first domain binds to TRAV1-2 and the antigen binding domain of the third domain binds to TRAV1-2. In some embodiments, the first domain binds to TRBV20 and the antigen binding domain of the third domain binds to TRBV20. In some embodiments, the first domain binds to TRBV6 and the antigen binding domain of the third domain binds to TRBV6. In some embodiments, the first domain binds to both TRBV20 and TRBV6 and the antigen binding domain of the third domain binds to both TRBV20 and TRBV6.

In some embodiments, the first domain binds to TRAV1-2 and the antigen binding domain of the second and the third domain binds to TRAV1-2. In some embodiments, the first domain binds to TRBV20 and the antigen binding domain of the second and the third domain binds to TRBV20. In some embodiments, the first domain binds to TRBV6 and the antigen binding domain of the second and the third domain binds to TRBV6. In some embodiments, the first domain binds to both TRBV20 and TRBV6 and the antigen binding domain of the second and the third domain binds to both TRBV20 and TRBV6.

In some embodiments, the first domain binds to TRAV1-2 and the antigen binding domain of the second domain binds to TRBV6. In some embodiments, the first domain binds to TRAV1-2 and the antigen binding domain of the second domain binds to TRBV20. In some embodiments, the first domain binds to TRAV1-2 and the antigen binding domain of the second domain binds to both TRBV20 and TRBV6. In some embodiments, the first domain binds to TRBV20 and the antigen binding domain of the second domain binds to TRAV1-2. In some embodiments, the first domain binds to TRBV20 and the antigen binding domain of the second domain binds to TRBV6. In some embodiments, the first domain binds to TRBV20 and the antigen binding domain of the second domain binds to both TRBV20 and TRBV6. In some embodiments, the first domain binds to TRBV6 and the antigen binding domain of the second domain binds to TRAV1-2. In some embodiments, the first domain binds to TRBV6 and the antigen binding domain of the second domain binds to TRBV20. In some embodiments, the first domain binds to TRBV6 and the antigen binding domain of the second domain binds to both TRBV20 and TRBV6. In some embodiment, the first domain binds to both TRBV20 and TRBV6 and the antigen binding domain of the second domain binds to TRAV1-2. In some embodiments, the first domain binds to both TRBV20 and TRBV6 and the antigen binding domain of the second domain binds to TRBV20. In some embodiments, the first domain binds to both TRBV20 and TRBV6 and the antigen binding domain of the second domain binds to TRBV6.

In some embodiments, the first domain binds to TRAV1-2 and the antigen binding domain of the third domain binds to TRBV6. In some embodiments, the first domain binds to TRAV1-2 and the antigen binding domain of the third domain binds to TRBV20. In some embodiments, the first domain binds to TRAV1-2 and the antigen binding domain of the third domain binds to both TRBV20 and TRBV6. In some embodiments, the first domain binds to TRBV20 and the antigen binding domain of the third domain binds to TRAV1-2. In some embodiments, the first domain binds to TRBV20 and the antigen binding domain of the third domain binds to TRBV6. In some embodiments, the first domain binds to TRBV20 and the antigen binding domain of the third domain binds to both TRBV20 and TRBV6. In some embodiments, the first domain binds to TRBV6 and the antigen binding domain of the third domain binds to TRAV1-2. In some embodiments, the first domain binds to TRBV6 and the antigen binding domain of the third domain binds to TRBV20. In some embodiments, the first domain binds to TRBV6 and the antigen binding domain of the third domain binds to both TRBV20 and TRBV6. In some embodiment, the first domain binds to both TRBV20 and TRBV6 and the antigen binding domain of the third domain binds to TRAV1-2. In some embodiments, the first domain binds to both TRBV20 and TRBV6 and the antigen binding domain of the third domain binds to TRBV20. In some embodiments, the first domain binds to both TRBV20 and TRBV6 and the antigen binding domain of the third domain binds to TRBV6.

In some embodiments, the first domain binds to TRAV1-2, the antigen binding domain of the second domain binds to TRAV1-2, and the antigen binding domain of the third domain binds to TRBV1-2. In some embodiments, the first domain binds to TRAV1-2, the antigen binding domain of the second domain binds to TRAV1-2, and the antigen binding domain of the third domain binds to TRBV20. In some embodiments, the first domain binds to TRAV1-2, the antigen binding domain of the second domain binds to TRAV1-2, and the antigen binding domain of the third domain binds to TRBV6. In some embodiments, the first domain binds to TRAV1-2, the antigen binding domain of the second domain binds to TRAV1-2, and the antigen binding domain of the third domain binds to TRBV20 and TRBV6.

In some embodiments, the first domain binds to TRAV1-2, the antigen binding domain of the second domain binds to TRBV6, and the antigen binding domain of the third domain binds to TRBV1-2. In some embodiments, the first domain binds to TRAV1-2, the antigen binding domain of the second domain binds to TRBV6, and the antigen binding domain of the third domain binds to TRBV20. In some embodiments, the first domain binds to TRAV1-2, the antigen binding domain of the second domain binds to TRBV6, and the antigen binding domain of the third domain binds to TRBV6. In some embodiments, the first domain binds to TRAV1-2, the antigen binding domain of the second domain binds to TRAV6, and the antigen binding domain of the third domain binds to TRBV20 and TRBV6.

In some embodiments, the first domain binds to TRAV1-2, the antigen binding domain of the second domain binds to TRBV20, and the antigen binding domain of the third domain binds to TRBV1-2. In some embodiments, the first domain binds to TRAV1-2, the antigen binding domain of the second domain binds to TRBV20, and the antigen binding domain of the third domain binds to TRBV20. In some embodiments, the first domain binds to TRAV1-2, the antigen binding domain of the second domain binds to TRBV20, and the antigen binding domain of the third domain binds to TRBV6. In some embodiments, the first domain binds to TRAV1-2, the antigen binding domain of the second domain binds to TRBV20, and the antigen binding domain of the third domain binds to TRBV20 and TRBV6.

In some embodiments, the first domain binds to TRAV1-2, the antigen binding domain of the second domain binds to TRBV20 and TRBV6, and the antigen binding domain of the third domain binds to TRBV1-2. In some embodiments, the first domain binds to TRAV1-2, the antigen binding domain of the second domain binds to TRBV20 and TRBV6, and the antigen binding domain of the third domain binds to TRBV20. In some embodiments, the first domain binds to TRAV1-2, the antigen binding domain of the second domain binds to TRBV20 and TRBV6, and the antigen binding domain of the third domain binds to TRBV6. In some embodiments, the first domain binds to TRAV1-2, the antigen binding domain of the second domain binds to TRBV20 and TRBV6, and the antigen binding domain of the third domain binds to TRBV20 and TRBV6.

In some embodiments, the first domain binds to TRBV20, the antigen binding domain of the second domain binds to TRAV1-2, and the antigen binding domain of the third domain binds to TRBV1-2. In some embodiments, the first domain binds to TRBV20, the antigen binding domain of the second domain binds to TRAV1-2, and the antigen binding domain of the third domain binds to TRBV20. In some embodiments, the first domain binds to TRBV20, the antigen binding domain of the second domain binds to TRAV1-2, and the antigen binding domain of the third domain binds to TRBV6. In some embodiments, the first domain binds to TRBV20, the antigen binding domain of the second domain binds to TRAV1-2, and the antigen binding domain of the third domain binds to TRBV20 and TRBV6.

In some embodiments, the first domain binds to TRBV20, the antigen binding domain of the second domain binds to TRBV6, and the antigen binding domain of the third domain binds to TRBV1-2. In some embodiments, the first domain binds to TRBV20, the antigen binding domain of the second domain binds to TRBV6, and the antigen binding domain of the third domain binds to TRBV20. In some embodiments, the first domain binds to TRBV20, the antigen binding domain of the second domain binds to TRBV6, and the antigen binding domain of the third domain binds to TRBV6. In some embodiments, the first domain binds to TRBV20, the antigen binding domain of the second domain binds to TRAV6, and the antigen binding domain of the third domain binds to TRBV20 and TRBV6.

In some embodiments, the first domain binds to TRBV20, the antigen binding domain of the second domain binds to TRBV20, and the antigen binding domain of the third domain binds to TRBV1-2. In some embodiments, the first domain binds to TRBV20, the antigen binding domain of the second domain binds to TRBV20, and the antigen binding domain of the third domain binds to TRBV20. In some embodiments, the first domain binds to TRBV20, the antigen binding domain of the second domain binds to TRBV20, and the antigen binding domain of the third domain binds to TRBV6. In some embodiments, the first domain binds to TRBV20, the antigen binding domain of the second domain binds to TRBV20, and the antigen binding domain of the third domain binds to TRBV20 and TRBV6.

In some embodiments, the first domain binds to TRBV20, the antigen binding domain of the second domain binds to TRBV20 and TRBV6, and the antigen binding domain of the third domain binds to TRBV1-2. In some embodiments, the first domain binds to TRBV20, the antigen binding domain of the second domain binds to TRBV20 and TRBV6, and the antigen binding domain of the third domain binds to TRBV20. In some embodiments, the first domain binds to TRBV20, the antigen binding domain of the second domain binds to TRBV20 and TRBV6, and the antigen binding domain of the third domain binds to TRBV6. In some embodiments, the first domain binds to TRBV20, the antigen binding domain of the second domain binds to TRBV20 and TRBV6, and the antigen binding domain of the third domain binds to TRBV20 and TRBV6.

In some embodiments, the first domain binds to TRBV6, the antigen binding domain of the second domain binds to TRAV1-2, and the antigen binding domain of the third domain binds to TRBV1-2. In some embodiments, the first domain binds to TRBV6, the antigen binding domain of the second domain binds to TRAV1-2, and the antigen binding domain of the third domain binds to TRBV20. In some embodiments, the first domain binds to TRBV6, the antigen binding domain of the second domain binds to TRAV1-2, and the antigen binding domain of the third domain binds to TRBV6. In some embodiments, the first domain binds to TRBV6, the antigen binding domain of the second domain binds to TRAV1-2, and the antigen binding domain of the third domain binds to TRBV20 and TRBV6.

In some embodiments, the first domain binds to TRBV6, the antigen binding domain of the second domain binds to TRBV6, and the antigen binding domain of the third domain binds to TRBV1-2. In some embodiments, the first domain binds to TRBV6, the antigen binding domain of the second domain binds to TRBV6, and the antigen binding domain of the third domain binds to TRBV20. In some embodiments, the first domain binds to TRBV6, the antigen binding domain of the second domain binds to TRBV6, and the antigen binding domain of the third domain binds to TRBV6. In some embodiments, the first domain binds to TRBV6, the antigen binding domain of the second domain binds to TRAV6, and the antigen binding domain of the third domain binds to TRBV20 and TRBV6.

In some embodiments, the first domain binds to TRBV6, the antigen binding domain of the second domain binds to TRBV20, and the antigen binding domain of the third domain binds to TRBV1-2. In some embodiments, the first domain binds to TRBV6, the antigen binding domain of the second domain binds to TRBV20, and the antigen binding domain of the third domain binds to TRBV20. In some embodiments, the first domain binds to TRBV6, the antigen binding domain of the second domain binds to TRBV20, and the antigen binding domain of the third domain binds to TRBV6. In some embodiments, the first domain binds to TRBV6, the antigen binding domain of the second domain binds to TRBV20, and the antigen binding domain of the third domain binds to TRBV20 and TRBV6.

In some embodiments, the first domain binds to TRBV6, the antigen binding domain of the second domain binds to TRBV20 and TRBV6, and the antigen binding domain of the third domain binds to TRBV1-2. In some embodiments, the first domain binds to TRBV6, the antigen binding domain of the second domain binds to TRBV20 and TRBV6, and the antigen binding domain of the third domain binds to TRBV20. In some embodiments, the first domain binds to TRBV6, the antigen binding domain of the second domain binds to TRBV20 and TRBV6, and the antigen binding domain of the third domain binds to TRBV6. In some embodiments, the first domain binds to TRBV6, the antigen binding domain of the second domain binds to TRBV20 and TRBV6, and the antigen binding domain of the third domain binds to TRBV20 and TRBV6.

In some embodiments, the first domain binds to TRBV20 and TRBV6, the antigen binding domain of the second domain binds to TRAV1-2, and the antigen binding domain of the third domain binds to TRBV1-2. In some embodiments, the first domain binds to TRBV20 and TRBV6, the antigen binding domain of the second domain binds to TRAV1-2, and the antigen binding domain of the third domain binds to TRBV20. In some embodiments, the first domain binds to TRBV20 and TRBV6, the antigen binding domain of the second domain binds to TRAV1-2, and the antigen binding domain of the third domain binds to TRBV6. In some embodiments, the first domain binds to TRBV20 and TRBV6, the antigen binding domain of the second domain binds to TRAV1-2, and the antigen binding domain of the third domain binds to TRBV20 and TRBV6.

In some embodiments, the first domain binds to TRBV20 and TRBV6, the antigen binding domain of the second domain binds to TRBV6, and the antigen binding domain of the third domain binds to TRBV1-2. In some embodiments, the first domain binds to TRBV20 and TRBV6, the antigen binding domain of the second domain binds to TRBV6, and the antigen binding domain of the third domain binds to TRBV20. In some embodiments, the first domain binds to TRBV20 and TRBV6, the antigen binding domain of the second domain binds to TRBV6, and the antigen binding domain of the third domain binds to TRBV6. In some embodiments, the first domain binds to TRBV20 and TRBV6, the antigen binding domain of the second domain binds to TRAV6, and the antigen binding domain of the third domain binds to TRBV20 and TRBV6.

In some embodiments, the first domain binds to TRBV20 and TRBV6, the antigen binding domain of the second domain binds to TRBV20, and the antigen binding domain of the third domain binds to TRBV1-2. In some embodiments, the first domain binds to TRBV20 and TRBV6, the antigen binding domain of the second domain binds to TRBV20, and the antigen binding domain of the third domain binds to TRBV20. In some embodiments, the first domain binds to TRBV20 and TRBV6, the antigen binding domain of the second domain binds to TRBV20, and the antigen binding domain of the third domain binds to TRBV6. In some embodiments, the first domain binds to TRBV20 and TRBV6, the antigen binding domain of the second domain binds to TRBV20, and the antigen binding domain of the third domain binds to TRBV20 and TRBV6.

In some embodiments, the first domain binds to TRBV20 and TRBV6, the antigen binding domain of the second domain binds to TRBV20 and TRBV6, and the antigen binding domain of the third domain binds to TRBV1-2. In some embodiments, the first domain binds to TRBV20 and TRBV6, the antigen binding domain of the second domain binds to TRBV20 and TRBV6, and the antigen binding domain of the third domain binds to TRBV20. In some embodiments, the first domain binds to TRBV20 and TRBV6, the antigen binding domain of the second domain binds to TRBV20 and TRBV6, and the antigen binding domain of the third domain binds to TRBV6. In some embodiments, the first domain binds to TRBV20 and TRBV6, the antigen binding domain of the second domain binds to TRBV20 and TRBV6, and the antigen binding domain of the third domain binds to TRBV20 and TRBV6.

In some embodiments, the first domain binds to a MAIT cell associated marker disclosed herein and the second domain comprises a cytokine or functional fragment or variant thereof disclosed herein. In some embodiments, the first domain binds to a MAIT cell associated marker and the second domain binds to a TAA disclosed herein.

In some embodiments, the first domain binds to a MAIT cell associated marker disclosed herein and the third domain comprises a cytokine or functional fragment or variant thereof disclosed herein. In some embodiments, the first domain binds to a MAIT cell associated marker and the third domain binds to a TAA disclosed herein.

In some embodiments, the first domain binds to a MAIT cell associated marker disclosed herein, the second domain binds to a MAIT cell associated marker disclosed herein, and the third domain binds to a MAIT cell associated marker disclosed herein. In some embodiments, the first domain binds to a MAIT cell associated marker disclosed herein, the second domain binds to a MAIT cell associated marker disclosed herein, and the third domain comprises a cytokine or functional fragment or variant thereof disclosed herein. In some embodiments, the first domain binds to a MAIT cell associated marker disclosed herein, the second domain binds to a MAIT cell associated marker disclosed herein, and the third domain binds to a TAA disclosed herein.

In some embodiments, the first domain binds to a MAIT cell associated marker disclosed herein, the second domain binds to a TAA disclosed herein, and the third domain binds to a MAIT cell associated marker disclosed herein. In some embodiments, the first domain binds to a MAIT cell associated marker disclosed herein, the second domain binds to a TAA disclosed herein, and the third domain comprises a cytokine or functional fragment or variant thereof disclosed herein. In some embodiments, the first domain binds to a MAIT cell associated marker disclosed herein, the second domain binds to a TAA disclosed herein, and the third domain binds to a TAA disclosed.

In some embodiments, the first domain binds to a MAIT cell associated marker disclosed herein, the second domain comprises a cytokine or functional fragment or variant thereof disclosed herein, and the third domain binds to a MAIT cell associated marker disclosed herein. In some embodiments, the first domain binds to a MAIT cell associated marker disclosed herein, the second domain comprises a cytokine or functional fragment or variant thereof disclosed herein, and the third domain comprises a cytokine or functional fragment or variant thereof disclosed herein. In some embodiments, the first domain binds to a MAIT cell associated marker disclosed herein, the second domain comprises a cytokine or functional fragment or variant thereof disclosed herein, and the third domain binds to a TAA disclosed herein.

### Exemplary Structure 1

In some embodiments, the multispecific molecule comprises a first polypeptide and a second polypeptide. The first polypeptide and the second polypeptide can be contiguous or non-contiguous.

In some embodiments, the first polypeptide comprises a first portion of a dimerization module linked to the first domain that binds to a MAIT cell associated marker, e.g., TRAV1-2, TRBV20, TRBV6, and both TRBV20 and TRBV6, wherein the first domain that binds to TRAV1-2, TRBV20, TRBV6, or both TRBV20 and TRBV6 comprises an antigen binding domain, for example, a single chain variable fragment (scFv), or a single domain antibody (sdAb), VHH, nanobody, and DARPin.

In some embodiments, the first polypeptide comprises a first portion of the first domain that binds to a MAIT cell-associated marker, e.g., TRAV1-2, TRBV20, TRBV6, or both TRBV20 and TRBV6, wherein the first portion of the first domain that binds to TRAV1-2, TRBV20, TRBV6, or both TRBV20 and TRBV6 comprises a heavy chain variable domain (VH). The multispecific molecule can further comprise a third polypeptide comprising a second portion of the first domain that binds to a MAIT cell associated marker, e.g., TRAV1-2, TRBV20, TRBV6, or both TRBV20 and TRBV6, for example, a light chain variable domain (VL). The third polypeptide can be non-contiguous or contiguous with the first polypeptide and the second polypeptide.

The second polypeptide can comprise a second portion of the dimerization module. A cytokine or functional fragment or variant thereof can be covalently or non-covalently linked to the first polypeptide, the second polypeptide, or the third polypeptide. The antigen binding domain can be covalently or non-covalently linked to the first polypeptide, the second polypeptide, or the third polypeptide.

In some embodiments, the first polypeptide comprises the first portion of the first domain that binds to a MAIT cell associated marker, e.g., TRAV1-2, TRBV20, TRBV6, and both TRBV20 and TRBV6. In some embodiments, the first portion of the first domain can be linked to the N-terminus or the C-terminus of the first polypeptide comprising the first portion of the dimerization module. In some embodiments, the second polypeptide can be linked to the C-terminus or the C-terminus of a cytokine or a functional fragment or variant thereof.

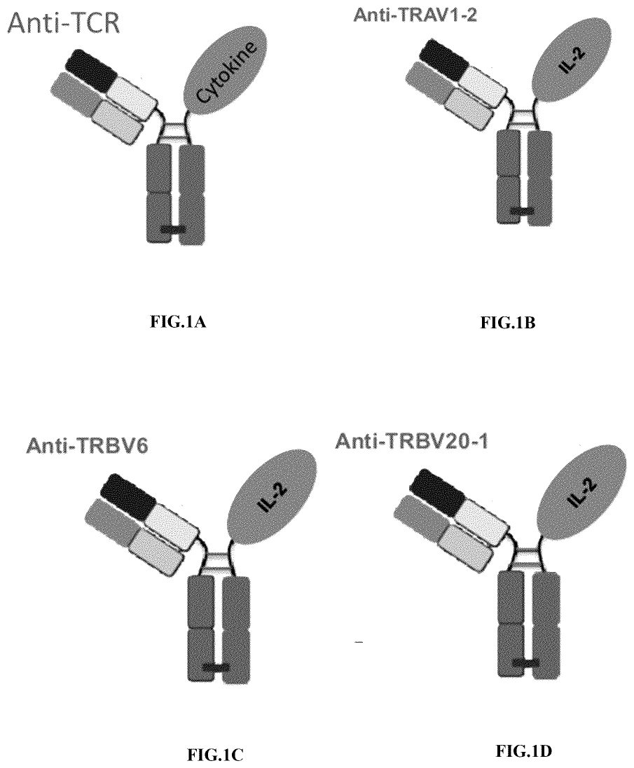

An illustrative structure is provided in FIG. 1. On the left-hand side of the molecule is a first antigen binding domain, in this case, a Fab comprising a VH and a VL The first antigen binding domain can bind to a MAIT associated marker disclosed herein or a TAA disclosed herein. The first antigen binding domain is linked to a first portion of the dimerization module, in this case, an immunoglobin heavy chain constant region comprising a CH2 and CH3 domain of IgG. The first portion of the dimerization module associates with a second portion of the dimerization module forming an Fc region. The second portion of the dimerization module is linked to a cytokine disclosed herein. The multispecific molecule comprises 3 polypeptide chains. The first polypeptide chain comprises one chain of the Fc region and VH of the first antigen binding domain. The second polypeptide chain comprises the other chain of the Fc region and the cytokine. The third polypeptide chain comprises the VL of the first antigen binding domain, which is associated with the VH as part of the Fab.

### Exemplary Structure 2

In some embodiments, the multispecific molecule comprises a first polypeptide and a second polypeptide. In some embodiments, the first polypeptide and the second polypeptide can be contiguous or non-contiguous.

In some embodiments, the first polypeptide comprises a first portion of a dimerization module linked to a third domain. In some embodiments, the third domain comprises an antigen binding domain, for example, a single chain variable fragment (scFv), a single domain antibody (sdAb), VHH, nanobody, and DARPin.

In some embodiments, the first polypeptide comprises a first portion of a dimerization module linked to a first portion of the third domain, wherein the first portion of the third domain comprises a heavy chain variable domain (VH). The multispecific molecule can further comprise a third polypeptide comprising a second portion of the third domain, for example, a light chain variable domain (VL). The third polypeptide can be contiguous and non-contiguous with the first polypeptide and the second polypeptide.

In some embodiments, the second polypeptide can comprise a second portion of the dimerization module, a cytokine or functional fragment or variant thereof, and the first domain. In some embodiments, the first domain comprises an antigen binding domain, for example, a single chain variable fragment (scFv), a single domain antibody (sdAb), VHH, nanobody, and DARPin. In some embodiments, the second polypeptide can comprise a second portion of the dimerization module, a cytokine or functional fragment or variant thereof, and a first portion of the first domain, for example, a heavy chain variable domain (VH). The multispecific molecule can further comprise a third polypeptide comprising a second portion of the first domain, for example, a light chain variable domain (VL). The third polypeptide can be contiguous non-contiguous with the first polypeptide and the second polypeptide.

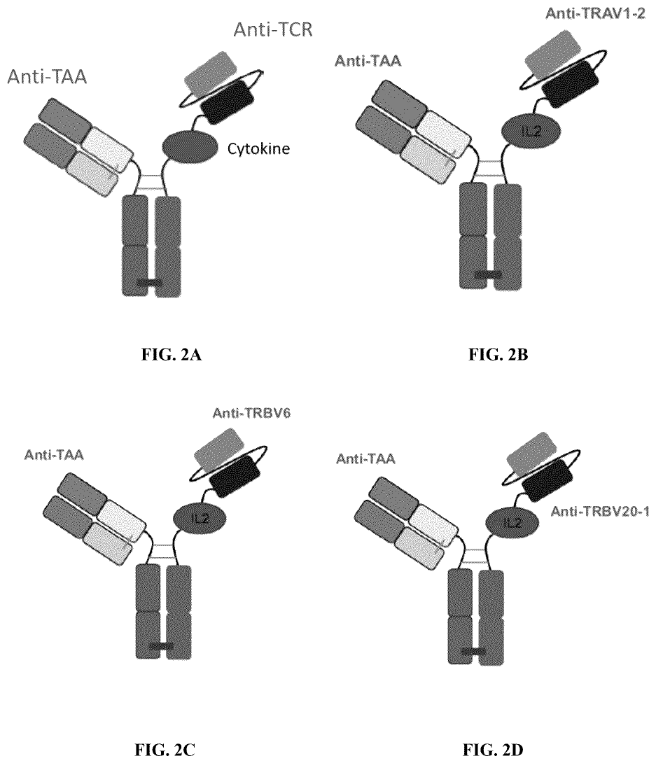

An illustrative structure is provided in FIG. 2. On the left-hand side of the molecule is a first antigen binding domain, in this case, a Fab comprising a VH and a VL. The first antigen binding domain can bind to a MAIT associated marker or a TAA. The first antigen binding domain is linked to a first portion of the dimerization module, in this case, an immunoglobin heavy chain constant region comprising a CH2 and CH3 domain of IgG. The first portion of the dimerization module associates with a second portion of the dimerization module forming an Fc region. The second portion of the dimerization module is linked to a cytokine and a second antigen binding domain, in this case, a scFv. The second antigen binding domain can bind to a MAIT associated marker or TAA. For example, if the first antigen binding domain binds to a MAIT associated marker, the second antigen binding domain binds to a TAA, or vice versa. The multispecific molecule comprises 3 polypeptide chains. The first polypeptide chain comprises one chain of the Fc region and VH of the first antigen binding domain. The second polypeptide chain comprises the other chain of the Fc region, the cytokine, and the VH and VL of the second antigen binding domain. The third polypeptide chain comprises the VL of the first antigen binding domain, which is associated with the VH as part of the Fab.

### Exemplary Structure 3

In some embodiments, the multispecific molecule comprises a first polypeptide and a second polypeptide. The first polypeptide and the second polypeptide can be contiguous or non-contiguous.

In some embodiments, the first polypeptide comprises a first portion of a dimerization module and the second domain comprising an antigen binding domain, for example, a single chain variable fragment (scFv), a single domain antibody (sdAb), VHH, nanobody, and DARPin. In some embodiments, the first polypeptide comprises a first portion of a dimerization module and a first portion of the second domain, for example, a heavy chain variable domain (VH). The multispecific molecule can further comprise a third polypeptide comprising a second portion of the second domain, for example, a light chain variable domain (VL). The third polypeptide can be contiguous and non-contiguous with the first polypeptide and the second polypeptide

In some embodiments, the second polypeptide comprises a second portion of the dimerization module and the first domain, for example, a single chain variable fragment (scFv), or a single domain antibody (sdAb), VHH, nanobody, and DARPin. In some embodiments, the second polypeptide comprises a second portion of the dimerization module and a first portion of the first domain, for example, a heavy chain variable domain (VH). The multispecific molecule can further comprise a third polypeptide comprising a second portion of the first domain, for example, a light chain variable domain (VL). The third polypeptide can be contiguous or non-contiguous with the first polypeptide and the second polypeptide.

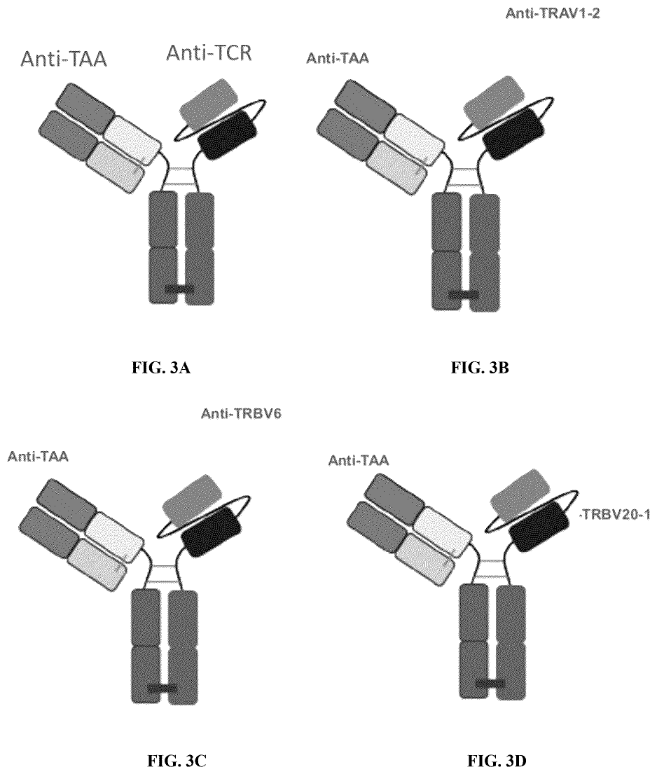

An illustrative structure is provided in FIG. 3. On the left-hand side of the molecule is a first antigen binding domain, in this case, a Fab comprising a VH and a VL. The first antigen binding domain can bind to a MAIT associated marker disclosed herein or a TAA disclosed herein. The first antigen binding domain is linked to a first portion of the dimerization module, in this case, an immunoglobin heavy chain constant region comprising a CH2 and CH3 domain of IgG. The first portion of the dimerization module associates with a second portion of the dimerization module forming an Fc region. The second portion of the dimerization module is linked to a second antigen binding domain, in this case, a scFv. The second antigen binding domain can bind to a MAIT associated marker or TAA. For example, if the first antigen binding domain binds to a MAIT associated marker, the second antigen binding domain binds to a TAA, or vice versa. The multispecific molecule comprises 3 polypeptide chains. The first polypeptide chain comprises one chain of the Fc region and VH of the first antigen binding domain. The second polypeptide chain comprises the other chain of the Fc region and the VH and VL of the second antigen binding domain. The third polypeptide chain comprises the VL of the first antigen binding domain, which is associated with the VH as part of the Fab.

### Exemplary Structure 4

The multispecific molecule comprises a first polypeptide and a second polypeptide. The first polypeptide and the second polypeptide can be contiguous and non-contiguous.

In some embodiments, the first polypeptide comprises a first portion of a dimerization module, the first domain or a first portion of the first domain, and the second domain or a first portion of the second domain.

In some embodiments, the first domain comprises a single chain variable fragment (scFv), a single domain antibody (sdAb), VHH, nanobody, or DARPin. In some embodiments, the first portion of the first domain comprises a heavy chain variable domain (VH). The multispecific molecule can further comprise a third polypeptide comprising a second portion of the first domain, for example, a light chain variable domain (VL). The third polypeptide can be contiguous and non-contiguous with the first polypeptide and the second polypeptide.

In some embodiments, the second domain comprises the cytokine or a functional fragment or variant thereof, or a single chain variable fragment (scFv), a single domain antibody (sdAb), VHH, nanobody, or DARPin. In some embodiments, the first portion of the second domain comprises a heavy chain variable domain (VH). The multispecific molecule can further comprises a fourth polypeptide comprising a second portion of the second domain, for example, a light chain variable domain (VL). The fourth polypeptide can be contiguous or non-contiguous with the first polypeptide and the second polypeptide. When multispecific molecule comprises the third polypeptide, the fourth polypeptide can be contiguous or non-contiguous with the third polypeptide.

In some embodiments, the second polypeptide comprises a second portion of the dimerization module, an additional first domain or a first portion of the additional first domain, and an additional second domain or a first portion of the additional second domain.

In some embodiments, the additional first domain comprises a single chain variable fragment (scFv), a single domain antibody (sdAb), VHH, nanobody, or DARPin. In some embodiments, the first portion of the additional first domain comprises a heavy chain variable domain (VH). The multispecific molecule can further comprise a fifth polypeptide comprising a second portion of the additional first domain, for example, a light chain variable domain (VL). The fifth polypeptide can be contiguous or non-contiguous with the first polypeptide and the second polypeptide. When the multispecific molecule comprises the third polypeptide and/or the fourth polypeptide, the fifth polypeptide can be contiguous or non-contiguous with the third polypeptide and the fourth polypeptide.

In some embodiments, the additional second domain comprises the cytokine or a functional fragment or variant thereof, or a single chain variable fragment (scFv), a single domain antibody (sdAb), VHH, nanobody, or DARPin. In some embodiments, the first portion of the additional second domain comprises a heavy chain variable domain (VH). The multispecific molecule can further comprise a sixth polypeptide comprising a second portion of the additional second domain, for example, a light chain variable domain (VL). The sixth polypeptide can be contiguous or non-contiguous with the first polypeptide and the second polypeptide. When the multispecific molecule comprises the third polypeptide, the fourth polypeptide, and/or the fifth polypeptide, the sixth polypeptide can be contiguous or non-contiguous with the third polypeptide, the fourth polypeptide, and the fifth polypeptide.

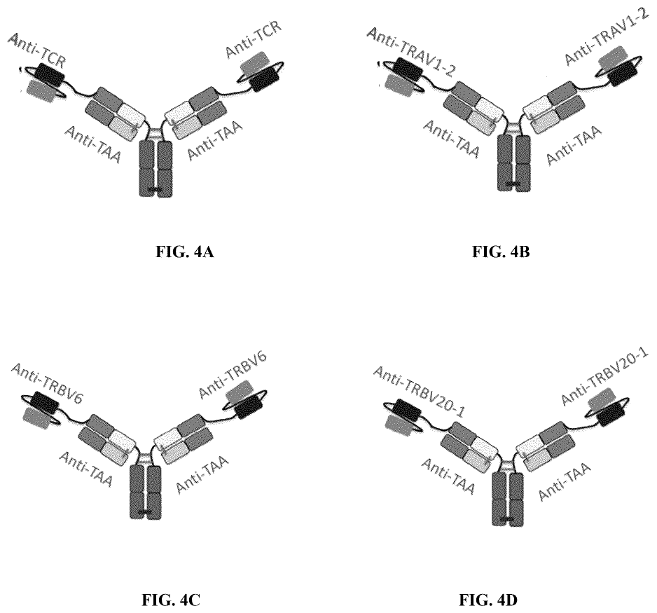

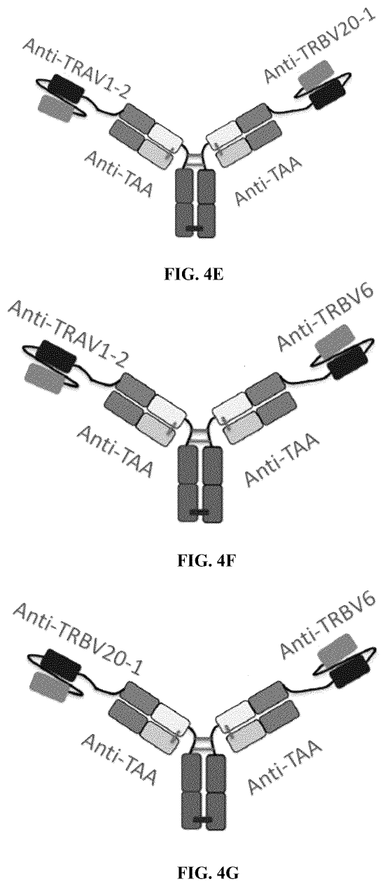

An illustrative structure is provided in FIG. 4. On the left-hand side of the molecule are a first antigen binding domain, in this case, a Fab comprising a VH and a V L, and a second antigen binding domain, in this case, a scFv, or vice versa. The first antigen binding domain can bind to a MAIT associated marker disclosed herein or a TAA disclosed herein. The second antigen binding domain can bind to a MAIT associated marker disclosed herein or a TAA disclosed herein. The first antigen binding domain and the second antigen binding domain is linked to a first portion of the dimerization module, in this case, an immunoglobin heavy chain constant region comprising a CH2 and CH3 domain of IgG. The first portion of the dimerization module associates with a second portion of the dimerization module forming an Fc region. The second portion of the dimerization module mirrors the first portion of the dimerization module. The multispecific molecule comprises 4 polypeptide chains. The first polypeptide chain comprises one chain of the Fc region, VH of the first antigen binding domain, and the VH and VL of the second antigen binding domain. Alternatively, the first polypeptide chain comprises one chain of the Fc region, VH of the second antigen binding domain, and the VH and VL of the first antigen binding domain. The second polypeptide chain comprises the other chain of the Fc region, VH of the first antigen binding domain, and the VH and VL of the second antigen binding domain. Alternatively, the second polypeptide chain comprises the other chain of the Fc region, VH of the second antigen binding domain, and the VH and VL of the first antigen binding domain. The third polypeptide chain comprises the VL of the first antigen binding domain, which is associated with the VH as part of the Fab. Alternatively, the third polypeptide chain comprises the VL of the second antigen binding domain, which is associated with the VH as part of the Fab. The fourth polypeptide chain comprises the VL of the first antigen binding domain, which is associated with the VH as part of the Fab. Alternatively, the fourth polypeptide chain comprises the VL of the second antigen binding domain, which is associated with the VH as part of the Fab.

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises a Fab, a F(ab′)2, an Fv, a single chain Fv (scFv), a single domain antibody, a diabody (dAb), a camelid antibody, and a combination thereof.

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises a Fab.

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises a F(ab′)2.

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises an Fv.

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises a single chain Fv (scFv).

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises a single domain antibody.

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises a diabody (dAb).

In some embodiments, the first domain that binds to a MAIT cell associated marker, e.g., TRAV1-2, TRBV20, TRBV6, and both TRBV20 and TRBV6, the second domain that binds to a MAIT cell associated marker, e.g., TRAV1-2, TRBV20, TRBV6, and both TRBV20 and TRBV6, or both comprises a camelid antibody.

### Linker

In some embodiments, the multifunctional polypeptide molecule as described herein further comprises a linker between the first portion of the first TCRαV- or TCRβV-binding moiety and the first dimerization module, a linker between the first VH and the first CHI, a linker between the first VL and the first CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 237 or SEQ ID NO: 306.

In some embodiments, the multifunctional polypeptide molecule as described herein further comprises a linker between the first TCRαV- or TCRβV-binding moiety and the first dimerization module, a linker between the second TCRαV- or TCRβV-binding moiety and the second dimerization module, a linker between the at least one cytokine polypeptide or the variant thereof and the first polypeptide, a linker between the at least one cytokine polypeptide or the variant thereof and the second polypeptide, or a combination thereof.

In some embodiments, the multifunctional polypeptide molecule as described herein further comprises a linker between the first TCRαV- or TCRβV-binding moiety and the first dimerization module, a linker between the at least one cytokine polypeptide or the variant thereof and the first polypeptide, a linker between the at least one cytokine polypeptide or the variant thereof and the second polypeptide, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 237 or SEQ ID NO: 306.

In some embodiments, the multifunctional polypeptide molecule as described herein further comprises a linker between the first portion of the first TCRαV- or TCRβV-binding moiety and the first dimerization module, a linker between the first portion of the second TCRαV- or TCRβV-binding moiety and the second dimerization module, a linker between the first VH and the first CHI, a linker between the first VL and the first CL, a linker between the second VH and the second CHI, a linker between the second VL and the second CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, a linker between the at least one cytokine polypeptide or the variant thereof and the fourth polypeptide, or a combination thereof. In some embodiments, the multifunctional polypeptide molecule as described herein further comprises a linker between the first portion of the first TCRαV- or TCRβV-binding moiety and the first dimerization module, a linker between the first VH and the first CHI, a linker between the first VL and the first CL, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 237 or SEQ ID NO: 306.

The multispecific or multifunctional molecule as described herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Ser. In some embodiments, the peptide linker is selected from GGGGS (SEQ ID NO: 259); GGGGSGGGGS (SEQ ID NO: 237); GGGGSGGGGSGGGGS (SEQ ID NO: 258); DVPSGPGGGGGSGGGGS (SEQ ID NO: 305); and GGGGSGGGGSGGGGGS (SEQ ID NO: 306). In some embodiments, the peptide linker is a A(EAAAK)nA (SEQ ID NO: 301) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 260); AEAAAKEAAAKEAAAKAAA (SEQ ID NO: 3315); AEAAAKEAAAKEAAAKEAAAKAAA (SEQ ID NO: 262); and AEAAAKEAAAKEAAAKEAAAKEAAAKAAA (SEQ ID NO: 263).

### Second Multispecific Molecule

A composition can comprise two or more multispecific molecules disclosed herein. The first multispecific molecule can comprise first, second, and third domain disclosed herein. The second multispecific can comprise fourth, fifth, and sixth domain, for example, corresponding to first, second, and third domain of a multispecific molecule disclosed herein that is not identical to the first multispecific.

In some embodiments, the second multispecific molecule comprises a fourth domain that binds to a MAIT cell associated marker disclosed herein, e.g., TRAV1-2, TRBV20, or TRBV6.

In some embodiments, the second multispecific molecule comprises a fifth domain. In some embodiments, the fifth domain comprises a cytokine or a functional fragment or variant thereof disclosed herein, or an antigen binding domain. In some embodiments, the antigen binding domain binds to a TAA disclosed herein or one or more of a MAIT cell associated marker disclosed herein, e.g., TRAV1-2, TRBV20, TRBV6, or both TRBV20 and TRBV6.

In some embodiments, the second multispecific molecule comprises a six domain. In some embodiments, the six domain comprises a cytokine or a functional fragment or variant thereof disclosed herein, or an antigen binding domain. In some embodiments, the antigen binding domain binds to a TAA disclosed herein or one or more of a MAIT cell associated marker disclosed herein, e.g., TRAV1-2, TRBV20, TRBV6, or both TRBV20 and TRBV6.

### Pharmaceutical Compositions

Described herein, in certain embodiments, is a pharmaceutical composition comprising the composition as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multifunctional or multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific molecules or the compositions as described herein, or a pharmaceutically acceptable salt, solvate, hydrate or ester thereof. The pharmaceutical formulation comprising the multifunctional or multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose). The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes. e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific molecules as described herein. In some embodiments, the multifunctional or multispecific molecules or the compositions as described herein is administered in combination with another drug or therapeutic agent.

### Method of Expanding a MAIT Cell

In another aspect, described herein is a method of expanding a population of cells comprising a MAIT cell comprises contacting the population of cells comprising the MAIT cell with any composition disclosed herein.

In another aspect, described herein is a method of expanding a population of cells comprising a MAIT cell comprising contacting the population of cells comprising the MAIT cell with a composition comprising a molecule, wherein the molecule comprises a first domain, and wherein the first domain binds to a target molecule, wherein the target molecule is TRAV1-2, TRBV20, TRBV6, or both TRBV20 and TRBV6, thereby expanding the MAIT cell.

In some embodiments, the MAIT cell can be CD8(+), CD4(+), CD4(−) CD8(−) double-negative (DN), or CD4(+) CD8(+) double-positive (DP). In some embodiments, the MAIT cell can be CD8(+). In some embodiments, the MAIT cell can be CD4(+). In some embodiments, the MAIT cell can be DN. In some embodiments, the MAIT cell can be DP.

The contacting can be in vivo. The contacting can be ex vivo. The contacting can be in vitro.

In some embodiments, the MAIT cell is obtained from a healthy subject. In some embodiments, the MAIT cell is obtained from a subject having a disease, e.g., a cancer, a solid tumor, an infection, an autoimmune disease as described herein.

In some embodiments, the method comprises expanding the MAIT cell at least 2-fold. In some embodiments, the method comprises expanding the MAIT cell at least 5-fold or at least 10-fold.

In some embodiments, a method of expansion as described herein results in an expansion of at least 1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 100, 200, 300, 400, 500, 600, 700, 800, 900, 1000, 1500, 2000, 2500, 3000, 3500, 400, 4500, 5000, 5500, 6000, 6500, 7000, 7500, 8000, 8500, 9000, 9500, or 10000 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 1,6 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks, or 8 weeks.

In some embodiments, the method comprises selectively expanding the MAIT cell.

In some embodiments, the method comprises expanding the MAIT cell, thereby producing an expanded cell population, wherein at least 5%, 10%, 20%, 30%, 40%, 50%, 60%, 70%, 80%, 90%, or 100% of the cells in the expanded population of cells are MAIT cells.

In some embodiments, the method comprises expanding the MAIT cell, thereby producing an expanded cell population, wherein at most 5%, 10%, 20%, 30%, 40%, 50%, 60%, 70%, 80%, 90%, or 100% of the cells in the expanded population of cells are non-MAIT cells.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, expansion of the population of MAIT cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TRAV or anti-TRBV antibody molecule or the multispecific or multifunctional molecules as described herein.

### Method of Activating a MAIT Cell

In another aspect, described herein is a method of activating a population of cells comprising a MAIT cell comprising contacting the population of cells comprising the MAIT cell with any composition disclosed herein.

In another aspect, described herein is a method of activating a population of cells comprising a MAIT cell comprising contacting the population of cells comprising the MAIT cell with a composition comprising a molecule, wherein the molecule comprises a first domain and a second domain, wherein the first domain binds to a target molecule, wherein the target molecule is TRAV1-2, TRBV20. TRBV6, or both TRBV20 and TRBV6, and wherein the second domain comprises a cytokine or a functional fragment or variant thereof, thereby activating the MAIT cell.

The contacting can be in vivo. The contacting can be ex vivo. The contacting can be in vitro.

In some embodiments, the MAIT cell is obtained from a healthy subject. In some embodiments, the MAIT cell is obtained from a subject having a disease, e.g., a cancer, a solid tumor, an infection, an autoimmune disease as described herein.

In some embodiments, the method specifically or preferentially activates the MAIT cell of the population of cells.

In some embodiments, the method comprises activating the MAIT cell, thereby producing an activated cell population.

In some embodiments, the activated cell population produces a higher level of IL-2, IL-17, IL-22, IL-26, IFNγ, or TNF compared to the level of the IL-2, IL-17, IL-22, IL-26, IFN-γ, or TNF produced by a cell population produced by contacting the population of cells comprising the MAIT cell with an anti-CD3 antibody at the same concentration, an anti-TRBV antibody that does not bind TRBV20, TRBV6 or both TRBV20 and TRBV6 at the same concentration, or an anti-TRAV antibody that does not bind TRAV1-2 at the same concentration according to a cytokine release assay, and a lower level of IL-4, IL-10, or IL-12 compared to the level of the IL-4. IL-10, or IL-12 produced by a cell population produced by contacting the population of cells comprising the MAIT cell with an anti-CD3 antibody at the same concentration, an anti-TRBV antibody that does not bind TRBV20, TRBV6 or both TRBV20 and TRBV6 at the same concentration, or an anti-TRAV antibody that does not bind TRAV1-2 at the same concentration according to a cytokine release assay.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-CD3 antibody at the same concentration, an anti-TRBV antibody that does not bind TRBV20, TRBV6 or both TRBV20 and TRBV6 at the same concentration, or an anti-TRAV antibody that does not bind TRAV1-2 at the same concentration according to a cytokine release assay, or a lower level of IL-4, IL-10, or IL-12 compared to the level of the IL-4, IL-10, or IL-12 produced by a cell population produced by contacting the population of cells comprising the MAIT cell with an anti-CD3 antibody at the same concentration, an anti-TRBV antibody that does not bind TRBV20, TRBV6 or both TRBV20 and TRBV6 at the same concentration, or an anti-TRAV antibody that does not bind TRAV1-2 at the same concentration according to a cytokine release assay.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-CD3 antibody at the same concentration, an anti-TRBV antibody that does not bind TRBV20, TRBV6 or both TRBV20 and TRBV6 at the same concentration, or an anti-TRAV antibody that does not bind TRAV1-2 at the same concentration according to a cytokine release assay. In some embodiments, the activated cell population produces a lower level of IL-4, IL-10, or IL-12 compared to the level of the IL-4, IL-10, or IL-12 produced by a cell population produced by contacting the population of cells comprising the MAIT cell with an anti-CD3 antibody at the same concentration, an anti-TRBV antibody that does not bind TRBV20, TRBV6 or both TRBV20 and TRBV6 at the same concentration, or an anti-TRAV antibody that does not bind TRAV1-2 at the same concentration according to a cytokine release assay.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-CD3 antibody at the same concentration.

In some embodiments, the activated cell population produces at least about 1%, 5%, 19%, 20%, 30%, 40%, 50%, 60%, 70%, 80%, 90%, or 100% higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-CD3 antibody at the same concentration.

In some embodiments, the activated cell population produces at most about 1%, 5%, 19%, 20%, 30%, 40%, 50%, 60%, 70%, 80%, 90%, or 100% higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-CD3 antibody at the same concentration.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRBV antibody that does not bind TRBV20 or TRBV6 at the same concentration.

In some embodiments, the activated cell population produces at least about 1%. 5%, 19%, 20%, 30%, 40%, 50%, 60%, 70%, 80%, 90%, or 100% higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRBV antibody that does not bind TRBV20 or TRBV6 at the same concentration.

In some embodiments, the activated cell population produces at most about 1%, 5%, 19%, 20%, 30%, 40%, 50%, 60%, 70%, 80%, 90%, or 100% higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRBV antibody that does not bind TRBV20 or TRBV6 at the same concentration.

In some embodiments, the activated cell population produces a higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRAV antibody that does not bind TRAV1-2 at the same concentration.

In some embodiments, the activated cell population produces at least about 1%, 5%, 19%, 20%, 30%, 40%, 50%, 60%, 70%, 80%, 90%, or 100% higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRAV antibody that does not bind TRAV1-2 at the same concentration.

In some embodiments, the activated cell population produces at most about 1%, 5%, 19%, 20%, 30%, 40%, 50%, 60%, 70%, 80%, 90%, or 100% higher level of IL-2, IL-17, IL-22, IL-26, IFN-γ, or TNF compared to the level of the IL-2, IL-17, IL-22, IL-26, IFN-γ, or TNF produced by a cell population produced by contacting the population of cells comprising the MAIT cell with an anti-TRAV antibody that does not bind TRAV1-2 at the same concentration.

In some embodiments, the activated cell population produces a lower level of IL-4, IL-10, or IL-12 compared to the level of the IL-4, IL-10, or IL-12 produced by a cell population produced by contacting the population of cells comprising the MAIT cell with an anti-CD3 antibody at the same concentration.

In some embodiments, the activated cell population produces a lower level of IL-4, IL-10, or IL-12 compared to the level of the IL-4, IL-10, or IL-12 produced by a cell population produced by contacting the population of cells comprising the MAIT cell with an anti-CD3 antibody at the same concentration.

In some embodiments, the activated cell population produces a higher level of IL-4, IL-10, or IL-12 compared to the level of the IL-4, IL-10, or IL-12 produced by a cell population produced by contacting the population of cells comprising the MAIT cell with an anti-CD3 antibody at the same concentration.

In some embodiments, the activated cell population produces at least about 1%, 5%, 19%, 20%, 30%, 40%, 50%, 60%, 70%, 80%, 90%, or 100% higher level of IL-4, IL-10, or IL-12 compared to the level of the IL-4, IL-10, or IL-12 produced by a cell population produced by contacting the population of cells comprising the MAIT cell with an anti-CD3 antibody at the same concentration.

In some embodiments, the activated cell population produces at most about 1%, 5%, 19%, 20%, 30%, 40%, 50%, 60%, 70%, 80%, 90%, or 100% higher level of IL-4, IL-10, or IL-12 compared to the level of the IL-4, IL-10, or IL-12 produced by a cell population produced by contacting the population of cells comprising the MAIT cell with an anti-CD3 antibody at the same concentration.

In some embodiments, the activated cell population produces a higher level of IL-4, IL-10, or IL-12 compared to the level of the IL-4, IL-10, or IL-12 produced by a cell population produced by contacting the population of cells comprising the MAIT cell with an anti-TRBV antibody that does not bind TRBV20 or TRBV6 at the same concentration.

In some embodiments, the activated cell population produces at least about 1%, 5%, 19%, 20%, 30%, 40%, 50%, 60%, 70%, 80%, 90%, or 100% higher level of IL-4, IL-10, or IL-12 compared to the level of the IL-4, IL-10, or IL-12 produced by a cell population produced by contacting the population of cells comprising the MAIT cell with an anti-TRBV antibody that does not bind TRBV20 or TRBV6 at the same concentration.

In some embodiments, the activated cell population produces at most about 1%. 5%, 19%, 20%, 30%, 40%, 50%, 60%, 70%, 80%, 90%, or 100% higher level of IL-4, IL-10, or IL-12 compared to the level of the IL-4, IL-10, or IL-12 produced by a cell population produced by contacting the population of cells comprising the MAIT cell with an anti-TRBV antibody that does not bind TRBV20 or TRBV6 at the same concentration.

In some embodiments, the activated cell population produces a higher level of IL-4, IL-10, or IL-12 compared to the level of the IL-4, IL-10, or IL-12 produced by a cell population produced by contacting the population of cells comprising the MAIT cell with an anti-TRAV antibody that does not bind TRAV1-2 at the same concentration.

In some embodiments, the activated cell population produces at least about 1%, 5%, 19%, 20%, 30%, 40%, 50%, 60%, 70%, 80%, 90%, or 100% higher level of IL-4, IL-10, or IL-12 compared to the level of the IL-4, IL-10, or IL-12 produced by a cell population produced by contacting the population of cells comprising the MAIT cell with an anti-TRAV antibody that does not bind TRAV1-2 at the same concentration.

In some embodiments, the activated cell population produces at most about 1%, 5%, 19%, 20%, 30%, 40%, 50%, 60%, 70%, 80%, 90%, or 100% higher level of IL-4, IL-10, or IL-12 compared to the level of the IL-4, IL-10, or IL-12 produced by a cell population produced by contacting the population of cells comprising the MAIT cell with an anti-TRAV antibody that does not bind TRAV1-2 at the same concentration.

In some embodiments, the MAIT cell expresses TRAV1-2, TRBV20, TRBV6, or both TRBV20 and TRBV6 In some embodiments, the MAIT cell is CD45RA⁻CD45RO⁺CD95^{Hi}CD62L^{Lo}CD44^{Hi}.

In some embodiments, the method is performed in vivo. In some embodiments, the method is performed in vitro. In some embodiments, the method is performed ex vivo.

### Method of Treatment

In another aspect, described herein is a method of treating a disease or condition in a subject in need thereof comprising administering to the subject a therapeutically effective amount of any composition disclosed herein or any pharmaceutical composition disclosed herein.

In another aspect, described herein is a method of treating a disease or condition in a subject in need thereof comprising administering to the subject a therapeutically effective amount of a composition, for example, a molecule to activate or expand a MAIT cell of the subject. The molecule comprises a first domain and a second domain, wherein the first domain binds to TRAV1-2, TRBV20, TRBV6, or both TRBV20 and TRBV6, and wherein the second domain comprises a cytokine or a functional fragment or variant thereof.

In some embodiments, the method further comprises administering a third therapeutic agent or therapy to the subject. In some embodiments, the third therapeutic agent or therapy comprises a chemotherapeutic agent, a biologic agent, a hormonal therapy, radiation, or surgery.

In some embodiments, the third therapeutic agent or therapy is administered in combination with one or more of the molecules of any one of the preceding embodiments sequentially, simultaneously, or concurrently.

In some embodiments, the disease or condition is a cancer. In some embodiments, the cancer is a solid tumor, a hematological cancer, a metastatic cancer, a soft tissue tumor, or any combination thereof.

In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and any combination thereof.

In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof.

In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and any combination thereof.

In some embodiments, the T-cell lymphoma is peripheral T-cell lymphoma.

In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA, CD19, CD20, CD22, FcRH5, PDL1, CD47, gangloside 2 (GD2), prostate stem cell anti-gen (PSCA), prostate specific membrane antigen (PMSA), prostate-specific antigen (PSA), carcinoembryonic antigen (CEA), Ron Kinase, c-Met, Immature laminin receptor. TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUT1, RU12, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

Methods described herein include treating a cancer in a subject by using multispecific or multifunctional molecules as described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the disease or condition is an infection, for example, a bacterial, viral, or fungal infection. In some embodiments, the infection is selected from the group consisting of a S. parathyphi infection, a Bacteroidetes infection, a Proteobacteria infection, a M. tuberculosis infection, a respiratory virus infection, a SARS-CoV-2 infection, yellow fever, influenza, a HIV infection, a Cytomegalovirus infection, and an Epstein-Barr Virus infection.

In some embodiments, the disease or condition is an autoimmune disease. In some embodiments, the autoimmune diseases is selected from the group consisting of multiple sclerosis, Crohn's disease, Sjögren's syndrome, celiac disease, diabetes, arthritis, and Lupus, systemic sclerosis, osteopetrosis, inherited metabolic disorders, adrenoleukodystrophy, amegakaryocytic thrombocytopenia, sickle cell disease, severe congenital immunodeficiency, Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome.

### Tumor Antigen Moiety

In some embodiments, the multifunctional molecule further includes a tumor antigen moiety. In some embodiments, the tumor-targeting moiety is an antigen, e.g., a cancer antigen. In some embodiments, the cancer antigen is a tumor antigen or stromal antigen, or a hematological antigen.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

In some embodiments, the tumor-targeting moiety, e.g., cancer antigen, is chosen from: BCMA, FcRH5, CD19, CD20, CD22, CD30, CD33, CD38, CD47, CD99, CD123, FcRH5. CLEC12, CD179A, SLAMF7, or NY-ESO1, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUT1, RU12, SAGE, TRG, TRP1, TSTA, Folate receptor alpha, L1-CAM, CAIX, gpA33, GD3, GM2, VEGFR, Intergrins (Integrin alphaVbeta3, Integrin alpha5Beta1), Carbohydrates (Le), IGF1R, EPHA3, TRAILR1, TRAILR2, RANKL, (FAP), TGF-beta, hyaluronic acid, collagen, e.g., collagen IV, tenascin C, or tenascin W. In some embodiments, the tumor-targeting moiety, e.g., cancer antigen, is BCMA. In some embodiments, the tumor-targeting moiety, e.g., cancer antigen, is FcRH5.

In some embodiments, the tumor-targeting moiety, e.g., cancer antigen, is chosen from: CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117). Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK). Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1. Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1).

### FcRH5 Targeting Moieties.

In some embodiments, the multispecific molecules as described herein include a targeting moiety that binds to FcRH5 (e.g., a FcRH5 targeting moiety). The FcRH5 targeting moiety can be chosen from an antibody molecule (e.g., an antigen binding domain as described herein), a receptor or a receptor fragment, or a ligand or a ligand fragment, or a combination thereof. In some embodiments, the FcRH5 targeting moiety associates with, e.g., binds to, a cancer or hematopoietic cell (e.g., a molecule, e.g., antigen, present on the surface of the cancer or hematopoietic cell). In certain embodiments, the FcRH5 targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the FcRH5 targeting moiety, binds to a FcRH5 antigen on the surface of a cell, e.g., a cancer or hematopoietic cell. The FcRH5 antigen can be present on a primary tumor cell, or a metastatic lesion thereof. In some embodiments, the cancer is a hematological cancer, e.g., multiple myeloma. For example, the FcRH5 antigen can be present on a tumor, e.g., a tumor of a class typified by having one or more of: limited tumor perfusion, compressed blood vessels, or fibrotic tumor interstitium.

The multispecific molecules described herein includes a FcRH5 targeting moiety that comprises an anti-FcRH5 antibody or antigen-binding fragment thereof described in U.S. Pat. No. 7,999,077, US20150098900, U.S. Pat. Nos. 8,299,220, 7,105,149, 8,362,213, 8,466,260, 8,617,559, US20160368985, US20150166661, and US20080247944, the entire contents of any of the aforesaid publications are herein incorporated by reference.

In some embodiments, the multispecific molecules described herein includes a FcRH5 targeting moiety that comprises an anti-FcRH5 antibody or antigen-binding fragment thereof described in U.S. Pat. No. 7,999,077, the entire contents of which are herein incorporated by reference.

### BCMA Targeting Moieties:

In certain embodiments, the multispecific molecules as described herein include a targeting moiety that binds to BCMA (e.g., a BCMA targeting moiety). The BCMA targeting moiety can be chosen from an antibody molecule (e.g., an antigen binding domain as described herein), a receptor or a receptor fragment, or a ligand or a ligand fragment, or a combination thereof. In some embodiments, the BCMA targeting moiety associates with, e.g., binds to, a cancer or hematopoietic cell (e.g., a molecule, e.g., antigen, present on the surface of the cancer or hematopoietic cell). In certain embodiments, the BCMA targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the BCMA targeting moiety, binds to a BCMA antigen on the surface of a cell, e.g., a cancer or hematopoietic cell. The BCMA antigen can be present on a primary tumor cell, or a metastatic lesion thereof. In some embodiments, the cancer is a hematological cancer, e.g., multiple myeloma. For example, the BCMA antigen can be present on a tumor, e.g., a tumor of a class typified by having one or more of: limited tumor perfusion, compressed blood vessels, or fibrotic tumor interstitium.

Exemplary BCMA targeting moieties: the multispecific molecules described herein can include a BCMA targeting moiety that comprises an anti-BCMA antibody or antigen-binding fragment thereof described in U.S. Pat. Nos. 8,920,776, 9,243,058, 9,340,621, 8,846,042, 7,083,785, 9,545,086, 7,276,241, 9,034,324, 7,799,902, 9,387,237, 8,821,883, 861,745, US20130273055, US20160176973, US20150368351, US20150376287, US20170022284, US20160015749, US20140242077, US20170037128, US20170051068, US20160368988, US20160311915, US20160131654, US20120213768, US20110177093, US20160297885, EP3137500, EP2699259, EP2982694, EP3029068, EP3023437, WO2016090327, WO2017021450, WO2016110584, WO2016118641, WO2016168149, the entire contents of which are incorporated herein by reference.

In some embodiments, the BCMA-targeting moiety includes an antibody molecule (e.g., Fab or scFv) that binds to BCMA. In some embodiments, the antibody molecule to BCMA comprises one, two, or three CDRs from any of the heavy chain variable domain sequences of Table 14, or a closely related CDR, e.g., CDRs which have at least one amino acid alteration, but not more than two, three or four alterations (e.g., substitutions, deletions, or insertions, e.g., conservative substitutions) from any of the CDR sequences of Table 14. In some embodiments, the antibody molecule to BCMA comprises a heavy chain variable domain sequence chosen from any of the amino acid sequences of Table 14, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

Alternatively, or in combination with the heavy chain to BCMA as described herein, the antibody molecule to BCMA comprises one, two, or three CDRs from any of the light chain variable domain sequences of Table 14, or a closely related CDR, e.g., CDRs which have at least one amino acid alteration, but not more than two, three or four alterations (e.g., substitutions, deletions, or insertions, e.g., conservative substitutions) from any of the CDR sequences of Table 14. In some embodiments, the antibody molecule to BCMA comprises a light chain variable domain sequence chosen from any of the amino acid sequences of Table 14, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

### Tumor-Targeting Moieties

In some embodiments, the multifunctional or multispecific (e.g., bi-, tri-, tetra-specific) molecules as described herein further include, e.g., are engineered to further contain, one or more tumor specific targeting moieties that direct the molecule to a tumor cell.

In certain embodiments, the multispecific molecules as described herein further include a tumor-targeting moiety. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules as described herein to a cancer (e.g., a cancer or tumor cells). In some embodiments, the cancer is chosen from a hematological cancer, a solid cancer, a metastatic cancer, or a combination thereof.

In some embodiments, the multispecific molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In some embodiments, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In other embodiments, the multispecific molecule, e.g., the tumor-targeting moiety, binds to a molecule, e.g., antigen, present on the surface of a hematological cancer, e.g., a leukemia or a lymphoma. In some embodiments, the hematological cancer is a B-cell or T cell malignancy. In some embodiments, the hematological cancer is chosen from one or more of a Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome (MDS), multiple myeloma, or acute lymphocytic leukemia. In embodiments, the cancer is other than acute myeloid leukemia (AML) or myelodysplastic syndrome (MDS). In embodiments, the hematological antigen is chosen from CD47, CD99, CD30, CD38, SLAMF7, or NY-ESO1. In some embodiments, the hematological antigen is chosen from is chosen from one or more of: BCMA, CD19, CD20, CD22, CD33, CD123, FcRH5, CLEC12, or CD179A.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the multifunctional or multispecific molecules or the compositions as described herein may vary depending on cell type to which delivery of the multifunctional or multispecific molecules or the compositions is desired. The multifunctional or multispecific molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multifunctional or multispecific molecules or the compositions as described herein are administered with one or more agents capable of promoting penetration of the subject the multifunctional or multispecific molecules or the compositions as described herein across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain.” incorporated herein by reference.

In some embodiments, the multifunctional or multispecific molecules or the compositions as described herein are linked or conjugated with agents that provide desirable pharmaceutical or pharmacodynamic properties. In some embodiments, the multifunctional or multispecific molecules or the compositions as described herein are coupled to a substance, known in the art to promote penetration or transport across the blood-brain barrier, e.g., an antibody to the transferrin receptor. In some embodiments, the multifunctional or multispecific molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antibody molecules, e.g., anti-TCRαV or anti-TCRβV antibody molecules, multispecific or multifunctional molecules are also disclosed.

Nucleic acids encoding the aforementioned recombinant T cell receptor or CAR are also disclosed.

In certain embodiments, the disclosure features nucleic acids comprising nucleotide sequences that encode heavy and light chain variable regions and CDRs or hypervariable loops of the antibody molecules, as described herein. For example, the disclosure features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions. e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions. e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%. 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRαV or anti-TCRβV antibody molecules, a multispecific or multifunctional molecule, or a CAR described herein. In some embodiments, the vectors comprise nucleic acid sequences encoding antibody molecules, e.g., anti-TCRαV or anti-TCRβV antibody molecules, multispecific or multifunctional molecule, or a CAR described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukarvotic cells include, but are not limited to, Vero cells, HeLa cells, COS cells, CHO cells, HEK293 cells, BHK cells and MDCKII cells. Suitable insect cells include, but are not limited to, SM9 cells.

Without wishing to be bound by theory, in some embodiments, the multispecific or multifunctional molecules as described herein can expand, e.g., selectively or preferentially expand, T cells expressing a T cell receptor (TCR) comprising a TCR alpha and/or TCR beta molecule, e.g., TCR alpha-beta T cells (4 T cells). In some embodiments, the multispecific or multifunctional molecules as described herein do not expand, or induce proliferation of T cells expressing a TCR comprising a TCR gamma and/or TCR delta molecule, e.g., TCR gamma-delta T cells (γδ T cells). In some embodiments, the multispecific or multifunctional molecules as described herein selectively or preferentially expand αβ T cells over γδ T cells.

Without wishing to be bound by theory, it is believed that, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g, an anti-IL-17 antibody or an agent that binds to a TCR gamma and/or TCR delta molecule.

TABLE 1
Amino acid and nucleotide sequences for murine, chimeric and humanized antibody
molecules which bind to TCRVB 6, e.g., TCRVB 6-5. The antibody molecules
include murine mAb Antibody A, and humanized mAb Antibody A-H Clones A-H.1 to
A-H.85. The amino acid the heavy and light chain CDRs, and the amino acid and
nucleotide sequences of the heavy and light chain variable regions, and the
heavy and light chains are shown.
Antibody A (murine), also referred to as H131, TCRVB 6-5 binder

| | | |
|---|---|---|
| SEQ ID NO: | HC CDR1 | GYSFTTYYIH |
| 3 | (Combined) | |
| SEQ ID NO: | HC CDR2 | WFFPGSGNIKYNEKFKG |
| 4 | (Combined) | |
| SEQ ID NO: | HC CDR3 | SYYSYDVLDY |
| 5 | (Combined) | |
| SEQ ID NO: | HC CDR1 (Kabat) | TYYIH |
| 45 | | |
| SEQ ID NO: | HC CDR2 (Kabat) | WFFPGSGNIKYNEKFKG |
| 4 | | |
| SEQ ID NO: | HC CDR3 (Kabat) | SYYSYDVLDY |
| 5 | | |
| SEQ ID NO: | HC CDR1 | GYSFTTY |
| 15 | (Chothia) | |
| SEQ ID NO: | HC CDR2 | FPGSGN |
| 16 | (Chothia) | |
| SEQ ID NO: | HC CDR3 | SYYSYDVLDY |
| 5 | (Chothia) | |
| SEQ ID NO: | VH | QVQLQQSGPELVKPGTSVKISCKASGYSFTTYYIH |
| 1 | | WVKQRPGQGLEWIGWFFPGSGNIKYNEKFKGKAT |
| | | LTADTSSSTAYMQLSSLTSEESAVYFCAGSYYSYD |
| | | VLDYWGHGTTLTVSS |
| SEQ ID NO: | LC CDR1 | KASQNVGINVV |
| 6 | (Combined) | |
| SEQ ID NO: | LC CDR2 | SSSHRYS |
| 7 | (Combined) | |
| SEQ ID NO: | LC CDR3 | QQFKSYPLT |
| 8 | (Combined) | |
| SEQ ID NO: | LC CDR1 (Kabat) | KASQNVGINVV |
| 6 | | |
| SEQ ID NO: | LC CDR2 (Kabat) | SSSHRYS |
| 7 | | |
| SEQ ID NO: | LC CDR3 (Kabat) | QQFKSYPLT |
| 8 | | |
| SEQ ID NO: | LC CDR1 | KASQNVGINVV |
| 6 | (Chothia) | |
| SEQ ID NO: | LC CDR2 | SSSHRYS |
| 7 | (Chothia) | |
| SEQ ID NO: | LC CDR3 | QQFKSYPLT |
| 8 | (Chothia) | |
| SEQ ID NO: | VL | DILMTQSQKFMSTSLGDRVSVSCKASQNVGINVV |
| 2 | | WHQQKPGQSPKALIYSSSHRYSGVPDRFTGSGSGT |
| | | DFTLTINNVQSEDLAEYFCQQFKSYPLTFGAGTKLE |
| | | LK |

Antibody A humanized (A-H antibody), TCRVB 6-5 binder
A-H.1 antibody (also referred to as BHM1709)

| | | |
|---|---|---|
| SEQ ID NO: | HC CDR1 | GYSFTTYYIH |
| 3 | (Combined) | |
| SEQ ID NO: | HC CDR2 | WFFPGSGNIKYNEKFKG |
| 4 | (Combined) | |
| SEQ ID NO: | HC CDR3 | SYYSYDVLDY |
| 5 | (Combined) | |
| SEQ ID NO: | VH | QVQLVQSGAEVKKPGSSVKVSCKASGYSFTTYYIH |
| 9 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| SEQ ID NO: | DNA VH | CAGGTGCAGCTGGTTCAGTCTGGCGCCGAAGTG |
| 12 | | AAGAAACCTGGCTCCTCCGTGAAGGTGTCCTGC |
| | | AAGGCTTCCGGCTACTCCTTCACCACCTACTACA |
| | | TCCACTGGGTCCGACAGGCCCCTGGACAAGGAT |
| | | TGGAATGGATGGGCTGGTTCTTCCCCGGCTCCGG |
| | | CAACATCAAGTACAACGAGAAGTTCAAGGGCCG |
| | | CGTGACCATCACCGCCGACACCTCTACCTCTACC |
| | | GCCTACATGGAACTGTCCAGCCTGAGATCTGAG |
| | | GACACCGCCGTGTACTACTGCGCCGGCTCCTACT |
| | | ACTCTTACGACGTGCTGGATTACTGGGGCCAGG |
| | | GCACCACAGTGACAGTGTCCTCT |
| SEQ ID NO: | VH-IgM constant | METDTLLLWVLLLWVPGSTGQVQLVQSGAEVKKP |
| 17 | delta CDC | GSSVKVSCKASGYSFTTYYIHWVRQAPGQGLEWM |
| | | GWFFPGSGNIKYNEKFKGRVTITADTSTSTAYMEL |
| | | SSLRSEDTAVYYCAGSYYSYDVLDYWGQGTTVTV |
| | | SSGSASAPTLFPLVSCENSPSDTSSVAVGCLAQDFL |
| | | PDSITFSWKYKNNSDISSTRGFPSVLRGGKYAATSQ |
| | | VLLPSKDVMQGTDEHVVCKVQHPNGNKEKNVPLP |
| | | VIAELPPKVSVFVPPRDGFFGNPRKSKLICQATGFSP |
| | | RQIQVSWLREGKQVGSGVTTDQVQAEAKESGPTT |
| | | YKVTSTLTIKESDWLGQSMFTCRVDHRGLTFQQN |
| | | ASSMCVPDQDTAIRVFAIPPSFASIFLTKSTKLTCLV |
| | | TDLTTYDSVTISWTRQNGEAVKTHTNISESHPNATF |
| | | SAVGEASICEDDWNSGERFTCTVTHTDLASSLKQTI |
| | | SRPKGVALHRPDVYLLPPAREQLNLRESATITCLVT |
| | | GFSPADVFVQWMQRGQPLSPEKYVTSAPMPEPQA |
| | | PGRYFAHSILTVSEEEWNTGETYTCVVAHEALPNR |
| | | VTERTVDKSTGKPTLYNVSLVMSDTAGTCY |
| SEQ ID NO: | VH-IgGA1 | METDTLLLWVLLLWVPGSTGQVQLVQSGAEVKKP |
| 18 | | GSSVKVSCKASGYSFTTYYIHWVRQAPGQGLEWM |
| | | GWFFPGSGNIKYNEKFKGRVTITADTSTSTAYMEL |
| | | SSLRSEDTAVYYCAGSYYSYDVLDYWGQGTTVTV |
| | | SSASPTSPKVFPLSLCSTQPDGNVVIACLVQGFFPQ |
| | | EPLSVTWSESGQGVTARNFPPSQDASGDLYTTSSQ |
| | | LTLPATQCLAGKSVTCHVKHYTNPSQDVTVPCPVP |
| | | STPPTPSPSTPPTPSPSCCHPRLSLHRPALEDLLLGSE |
| | | ANLTCTLTGLRDASGVTFTWTPSSGKSAVQGPPER |
| | | DLCGCYSVSSVLPGCAEPWNHGKTFTCTAAYPESK |
| | | TPLTATLSKSGNTFRPEVHLLPPPSEELALNELVTLT |
| | | CLARGFSPKDVLVRWLQGSQELPREKYLTWASRQ |
| | | EPSQGTTTFAVTSILRVAAEDWKKGDTFSCMVGHE |
| | | ALPLAFTQKTIDRLAGKPTHVNVSVVMAEVDGTC |
| | | Y |
| SEQ ID NO: | VH-IgGA2 | METDTLLLWVLLLWVPGSTGQVQLVQSGAEVKKP |
| 19 | | GSSVKVSCKASGYSFTTYYIHWVRQAPGQGLEWM |
| | | GWFFPGSGNIKYNEKFKGRVTITADTSTSTAYMEL |
| | | SSLRSEDTAVYYCAGSYYSYDVLDYWGQGTTVTV |
| | | SSASPTSPKVFPLSLDSTPQDGNVVVACLVQGFFPQ |
| | | EPLSVTWSESGQNVTARNFPPSQDASGDLYTTSSQ |
| | | LTLPATQCPDGKSVTCHVKHYTNSSQDVTVPCRVP |
| | | PPPPCCHPRLSLHRPALEDLLLGSEANLTCTLTGLR |
| | | DASGATFTWTPSSGKSAVQGPPERDLCGCYSVSSV |
| | | LPGCAQPWNHGETFTCTAAHPELKTPLTANITKSG |
| | | NTFRPEVHLLPPPSEELALNELVTLTCLARGFSPKD |
| | | VLVRWLQGSQELPREKYLTWASRQEPSQGTTTYA |
| | | VTSILRVAAEDWKKGETFSCMVGHEALPLAFTQK |
| | | TIDRMAGKPTHINVSVVMAEADGTCY |
| SEQ ID NO: | Heavy chain | METDTLLLWVLLLWVPGSTGQVQLVQSGAEVKKP |
| 20 | | GSSVKVSCKASGYSFTTYYIHWVRQAPGQGLEWM |
| | | GWFFPGSGNIKYNEKFKGRVTITADTSTSTAYMEL |
| | | SSLRSEDTAVYYCAGSYYSYDVLDYWGQGTTVTV |
| | | SSASTKGPSVFPLAPSSKSTSGGTAALGCLVKDYFP |
| | | EPVTVSWNSGALTSGVHTFPAVLQSSGLYSLSSVV |
| | | TVPSSSLGTQTYICNVNHKPSNTKVDKRVEPKSCD |
| | | KTHTCPPCPAPELLGGPSVFLFPPKPKDTLMISRTPE |
| | | VTCVVVDVSHEDPEVKFNWYVDGVEVHNAKTKP |
| | | REEQYNSTYRVVSVLTVLHQDWLNGKEYKCKVS |
| | | NKALPAPIEKTISKAKGQPREPQVYTLPPSREEMTK |
| | | NQVSLTCLVKGFYPSDIAVEWESNGQPENNYKTTP |
| | | PVLDSDGSFFLYSKLTVDKSRWQQGNVFSCSVMH |
| | | EALHNHYTQKSLSLSPGK |
| SEQ ID NO: | LC CDR1 | KASQNVGINVV |
| 6 | (Combined) | |
| SEQ ID NO: | LC CDR2 | SSSHRYS |
| 7 | (Combined) | |
| SEQ ID NO: | LC CDR3 | QQFKSYPLT |
| 8 | (Combined) | |
| SEQ ID NO: | VL | DIQMTQSPSFLSASVGDRVTITCKASQNVGINVVW |
| 10 | | HQQKPGKAPKALIYSSSHRYSGVPSRFSGSGSGTEF |
| | | TLTISSLQPEDFATYFCQQFKSYPLTFGQGTKLEIK |
| SEQ ID NO: | DNA VL | GACATCCAGATGACCCAGTCTCCATCCTTCCTGT |
| 13 | | CCGCCTCTGTGGGCGACAGAGTGACCATCACAT |
| | | GCAAGGCCTCTCAGAACGTGGGCATCAACGTCG |
| | | TGTGGCACCAGCAGAAGCCTGGCAAGGCTCCTA |
| | | AGGCTCTGATCTACTCCTCCAGCCACCGGTACTC |
| | | TGGCGTGCCCTCTAGATTTTCCGGCTCTGGCTCT |
| | | GGCACCGAGTTTACCCTGACAATCTCCAGCCTGC |
| | | AGCCTGAGGACTTCGCCACCTACTTTTGCCAGCA |
| | | GTTCAAGAGCTACCCTCTGACCTTTGGCCAGGGC |
| | | ACCAAGCTGGAAATCAAG |
| SEQ ID NO: | VL and kappa | METDTLLLWVLLLWVPGSTGDIQMTQSPSFLSASV |
| 23 | constant | GDRVTITCKASQNVGINVVWHQQKPGKAPKALIY |
| | region/light chain | SSSHRYSGVPSRFSGSGSGTEFTLTISSLQPEDFATY |
| | | FCQQFKSYPLTFGQGTKLEIKRTVAAPSVFIFPPSDE |
| | | QLKSGTASVVCLLNNFYPREAKVQWKVDNALQSG |
| | | NSQESVTEQDSKDSTYSLSSTLTLSKADYEKHKVY |
| | | ACEVTHQGLSSPVTKSFNRGEC |

A-H.2 antibody (also referred to as BHM1710)

| | | |
|---|---|---|
| SEQ ID NO: | HC CDR1 | GYSFTTYYIH |
| 3 | (Combined) | |
| SEQ ID NO: | HC CDR2 | WFFPGSGNIKYNEKFKG |
| 4 | (Combined) | |
| SEQ ID NO: | HC CDR3 | SYYSYDVLDY |
| 5 | (Combined) | |
| SEQ ID NO: | VH | QVQLVQSGAEVKKPGSSVKVSCKASGYSFTTYYIH |
| 9 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| SEQ ID NO: | DNA VH | CAGGTGCAGCTGGTTCAGTCTGGCGCCGAAGTG |
| 12 | | AAGAAACCTGGCTCCTCCGTGAAGGTGTCCTGC |
| | | AAGGCTTCCGGCTACTCCTTCACCACCTACTACA |
| | | TCCACTGGGTCCGACAGGCCCCTGGACAAGGAT |
| | | TGGAATGGATGGGCTGGTTCTTCCCCGGCTCCGG |
| | | CAACATCAAGTACAACGAGAAGTTCAAGGGCCG |
| | | CGTGACCATCACCGCCGACACCTCTACCTCTACC |
| | | GCCTACATGGAACTGTCCAGCCTGAGATCTGAG |
| | | GACACCGCCGTGTACTACTGCGCCGGCTCCTACT |
| | | ACTCTTACGACGTGCTGGATTACTGGGGCCAGG |
| | | GCACCACAGTGACAGTGTCCTCT |
| SEQ ID NO: | Heavy chain | METDTLLLWVLLLWVPGSTGQVQLVQSGAEVKKP |
| 20 | | GSSVKVSCKASGYSFTTYYIHWVRQAPGQGLEWM |
| | | GWFFPGSGNIKYNEKFKGRVTITADTSTSTAYMEL |
| | | SSLRSEDTAVYYCAGSYYSYDVLDYWGQGTTVTV |
| | | SSASTKGPSVFPLAPSSKSTSGGTAALGCLVKDYFP |
| | | EPVTVSWNSGALTSGVHTFPAVLQSSGLYSLSSVV |
| | | TVPSSSLGTQTYICNVNHKPSNTKVDKRVEPKSCD |
| | | KTHTCPPCPAPELLGGPSVFLFPPKPKDTLMISRTPE |
| | | VTCVVVDVSHEDPEVKFNWYVDGVEVHNAKTKP |
| | | REEQYNSTYRVVSVLTVLHQDWLNGKEYKCKVS |
| | | NKALPAPIEKTISKAKGQPREPQVYTLPPSREEMTK |
| | | NQVSLTCLVKGFYPSDIAVEWESNGQPENNYKTTP |
| | | PVLDSDGSFFLYSKLTVDKSRWQQGNVFSCSVMH |
| | | EALHNHYTQKSLSLSPGK |
| SEQ ID NO: | LC CDR1 | KASQNVGINVV |
| 6 | (Combined) | |
| SEQ ID NO: | LC CDR2 | SSSHRYS |
| 7 | (Combined) | |
| SEQ ID NO: | LC CDR3 | QQFKSYPLT |
| 8 | (Combined) | |
| SEQ ID NO: | VL | DIQMTQSPSSLSASVGDRVTITCKASQNVGINVVW |
| 11 | | HQQKPGKVPKALIYSSSHRYSGVPSRFSGSGSGTDF |
| | | TLTISSLQPEDVATYFCQQFKSYPLTFGQGTKLEIK |
| SEQ ID NO: | DNA VL | GACATCCAGATGACCCAGTCTCCATCCTCTCTGT |
| 14 | | CCGCCTCTGTGGGCGACAGAGTGACCATCACAT |
| | | GCAAGGCCTCTCAGAACGTGGGCATCAACGTCG |
| | | TGTGGCACCAGCAGAAACCTGGCAAGGTGCCCA |
| | | AGGCTCTGATCTACTCCTCCAGCCACAGATACTC |
| | | CGGCGTGCCCTCTAGATTCTCCGGCTCTGGCTCT |
| | | GGCACCGACTTTACCCTGACAATCTCCAGCCTGC |
| | | AGCCTGAGGACGTGGCCACCTACTTTTGCCAGCA |
| | | GTTCAAGAGCTACCCTCTGACCTTTGGCCAGGGC |
| | | ACCAAGCTGGAAATCAAG |
| SEQ ID NO: | Light chain | METDTLLLWVLLLWVPGSTGDIQMTQSPSSLSASV |
| 21 | | GDRVTITCKASQNVGINVVWHQQKPGKVPKALIY |
| | | SSSHRYSGVPSRFSGSGSGTDFTLTISSLQPEDVATY |
| | | FCQQFKSYPLTFGQGTKLEIKRTVAAPSVFIFPPSDE |
| | | QLKSGTASVVCLLNNFYPREAKVQWKVDNALQSG |
| | | NSQESVTEQDSKDSTYSLSSTLTLSKADYEKHKVY |
| | | ACEVTHQGLSSPVTKSFNRGEC |

A-H.3 antibody

| | | |
|---|---|---|
| SEQ ID NO: | VH + VL | QVQLVQSGAEVKKPGSSVKVSCKASGTDFKLTYIH |
| 22 | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVEDRVAW |
| 24 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 25 | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSS |

A-H.4

| | | |
|---|---|---|
| SEQ ID NO: | VH + VL | QVQLVQSGAEVKKPGSSVKVSCKASGTDFDKIYIH |
| 26 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVEDRVAW |
| 24 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKIYIH |
| 27 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |

A-H.5

| | | |
|---|---|---|
| SEQ ID NO: | VH + VL | QVQLVQSGAEVKKPGSSVKVSCKASGHDFRDFYI |
| 28 | | HWVRQAPGQGLEWMGRVYPGSGSYRYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFRDFYI |
| 30 | | HWVRQAPGQGLEWMGRVYPGSGSYRYNEKFKGR |
| | | VTITADTSTSTAYMELSSLRSEDTAVYYCAGSYYS |
| | | YDVLDYWGQGTTVTVSS |

A-H.6

| | | |
|---|---|---|
| SEQ ID NO: | VH + VL | QVQLVQSGAEVKKPGSSVKVSCKASGHDFKLTYI |
| 31 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDN |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDNRVAW |
| 32 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFKLTYI |
| 33 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSS |

A-H.7

| | | |
|---|---|---|
| SEQ ID NO: | VH + VL | QVQLVQSGAEVKKPGSSVKVSCKASGTDFKLTYIH |
| 34 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVENK |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVENKVAW |
| 35 | | HQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 36 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSS |

A-H.8

| | | |
|---|---|---|
| SEQ ID NO: | VH + VL | QVQLVQSGAEVKKPGSSVKVSCKASGTDFDKIYIH |
| 46 | | WVRQAPGQGLEWMGRIFAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKIYIH |
| 47 | | WVRQAPGQGLEWMGRIFAGSGNTKYNEKFKGRV |
| | | TITADTSTSTAYMELSSLRSEDTAVYYCAGSYYSY |
| | | DVLDYWGQGTTVTVSS |

A-H.9

| | | |
|---|---|---|
| SEQ ID NO: | VH + VL | QVQLVQSGAEVKKPGSSVKVSCKASGHDFDKFYI |
| 48 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVG |
| | | NRVAWYQQKPGKAPKALIYSSSHRYSGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVGNRVAW |
| 49 | | YQQKPGKAPKALIYSSSHRYSGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFDKFYI |
| 50 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSS |

A-H.10

| | | |
|---|---|---|
| SEQ ID NO: | VH + VL | QVQLVQSGAEVKKPGSSVKVSCKASGHDFDKFYI |
| 51 | | HWVRQAPGQGLEWMGRIFAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGD |
| | | RVAWYQQKPGKAPKALIYSSSHRYKGVPSRFSGSG |
| | | SGTEFTLTISSLQPEDFATYFCQQFKSYPLTFGQGT |
| | | KLEIKS |
| SEQ ID NO: | VL | DIQMTQSPSFLSASVGDRVTITCKASQNVGDRVAW |
| 52 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDEDKFYI |
| 53 | | HWVRQAPGQGLEWMGRIFAGSGNVKYNEKFKGR |
| | | VTITADTSTSTAYMELSSLRSEDTAVYYCAGSYYS |
| | | YDVLDYWGQGTTVTVSS |

A-H.11

| | | |
|---|---|---|
| SEQ ID NO: | VH + VL | QVQLVQSGAEVKKPGSSVKVSCKASGTDFKLTYIH |
| 54 | | WVRQAPGQGLEWMGRVSPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 55 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 56 | | WVRQAPGQGLEWMGRVSPGSGNVKYNEKFKGRV |
| | | TITADTSTSTAYMELSSLRSEDTAVYYCAGSYYSY |
| | | DVLDYWGQGTTVTVSS |

A-H.12

| | | |
|---|---|---|
| SEQ ID NO: | VH + VL | QVQLVQSGAEVKKPGSSVKVSCKASGTDFDKIYIH |
| 57 | | WVRQAPGQGLEWMGRVSAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGNRVAW |
| 58 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKIYIH |
| 59 | | WVRQAPGQGLEWMGRVSAGSGNTKYNEKFKGRV |
| | | TITADTSTSTAYMELSSLRSEDTAVYYCAGSYYSY |
| | | DVLDYWGQGTTVTVSS |

A-H.13, also referred to as A-H.69

| | | |
|---|---|---|
| SEQ ID NO: | VH + VL | QVQLVQSGAEVKKPGSSVKVSCKASGTDFKLTYIH |
| 60 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNRVAW |
| 32 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 36 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSS |

A-H.14

| | | |
|---|---|---|
| SEQ ID NO: | VH + VL | QVQLVQSGAEVKKPGSSVKVSCKASGTDFDKIYIH |
| 37 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKIYIH |
| 38 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSS |

A-H.15

| | | |
|---|---|---|
| SEQ ID NO: | VH + VL | QVQLVQSGAEVKKPGSSVKVSCKASGTDFRLTYIH |
| 61 | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNK |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNKVAW |
| 62 | | HQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFRLTYIH |
| 63 | | WVRQAPGQGLEWMGRVSPGSGNTKYNEKFKGRV |
| | | TITADTSTSTAYMELSSLRSEDTAVYYCAGSYYSY |
| | | DVLDYWGQGTTVTVSS |

A-H.16

| | | |
|---|---|---|
| SEQ ID NO: | VH + VL | QVQLVQSGAEVKKPGSSVKVSCKASGGTFRLTYIH |
| 64 | | WVRQAPGQGLEWMGRVYPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGGTFRLTYIH |
| 65 | | WVRQAPGQGLEWMGRVYPGSGNTKYNEKFKGRV |
| | | TITADTSTSTAYMELSSLRSEDTAVYYCAGSYYSY |
| | | DVLDYWGQGTTVTVSS |

A-H.17

| | | |
|---|---|---|
| SEQ ID NO: | VH + VL | QVQLVQSGAEVKKPGSSVKVSCKASGTDFRLTYIH |
| 66 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFRLTYIH |
| 67 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSS |

A-H.18

| | | |
|---|---|---|
| SEQ ID NO: | VH + VL | QVQLVQSGAEVKKPGSSVKVSCKASGTDFKLTYIH |
| 68 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVEDRVAW |
| 24 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 36 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSS |

A-H.19

| | | |
|---|---|---|
| SEQ ID NO: | VH + VL | QVQLVQSGAEVKKPGSSVKVSCKASGGTFRLTYIH |
| 69 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 55 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGGTFRLTYIH |
| 70 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |

A-H.20

| | | |
|---|---|---|
| SEQ ID NO: | VH + VL | QVQLVQSGAEVKKPGSSVKVSCKASGGTFDKTYI |
| 71 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGGTFDKTYI |
| 72 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSS |

A-H.21

| | | |
|---|---|---|
| SEQ ID NO: | VH + VL | QVQLVQSGAEVKKPGSSVKVSCKASGHDFDKFYI |
| 77 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFDKFYI |
| 78 | | HWVRQAPGQGLEWMGRISAGSGNTKYNEKFKGR |
| | | VTITADTSTSTAYMELSSLRSEDTAVYYCAGSYYS |
| | | YDVLDYWGQGTTVTVSS |

A-H.22

| | | |
|---|---|---|
| SEQ ID NO: | VH + VL | QVQLVQSGAEVKKPGSSVKVSCKASGTDFKLTYIH |
| 79 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNK |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNKVAW |
| 62 | | HQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 80 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |

A-H.23

| | | |
|---|---|---|
| SEQ ID NO: | VH + VL | QVQLVQSGAEVKKPGSSVKVSCKASGHDFRLTYIH |
| 81 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVADR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVADRVAW |
| 82 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFRLTYIH |
| 83 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |

A-H.24

| | | |
|---|---|---|
| SEQ ID NO: | VH + VL | QVQLVQSGAEVKKPGSSVKVSCKASGHDFHLWYI |
| 84 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVD |
| | | NKVAWHQQKPGKAPKALIYSSSHRYKGVPSRFSG |
| | | SGSGTEFTLTISSLQPEDFATYFCQQFKSYPLTFGQG |
| | | TKLEIK |
| SEQ ID NO: | VL | DIQMTQSPSFLSASVGDRVTITCKASQNVDNKVAW |
| 62 | | HQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFHLWYI |
| 85 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSS |

A-H.25

| | | |
|---|---|---|
| SEQ ID NO: | VH + VL | QVQLVQSGAEVKKPGSSVKVSCKASGHDFHLWYI |
| 86 | | HWVRQAPGQGLEWMGRVFAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVED |
| | | KVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVEDKVAW |
| 87 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFHLWYI |
| 88 | | HWVRQAPGQGLEWMGRVFAGSGNTKYNEKFKGR |
| | | VTITADTSTSTAYMELSSLRSEDTAVYYCAGSYYS |
| | | YDVLDYWGQGTTVTVSS |

A-H.26

| | | |
|---|---|---|
| SEQ ID NO: | VH + VL | QVQLVQSGAEVKKPGSSVKVSCKASGTDFKLTYIH |
| 89 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 90 | | WVRQAPGQGLEWMGRIFPGSGNTKYNEKFKGRV |
| | | TITADTSTSTAYMELSSLRSEDTAVYYCAGSYYSY |
| | | DVLDYWGQGTTVTVSS |

A-H.27

| | | |
|---|---|---|
| SEQ ID NO: | VH + VL | QVQLVQSGAEVKKPGSSVKVSCKASGTDFKLTYIH |
| 91 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGN |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVGNRVAW |
| 58 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 92 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSS |

A-H.28

| | | |
|---|---|---|
| SEQ ID NO: | VH + VL | QVQLVQSGAEVKKPGSSVKVSCKASGTDFKLTYIH |
| 93 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 55 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 94 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSS |

A-H.29

| | | |
|---|---|---|
| SEQ ID NO: | VH + VL | QVQLVQSGAEVKKPGSSVKVSCKASGHDFHLWYI |
| 95 | | HWVRQAPGQGLEWMGRISPGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGD |
| | | RVAWHQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVGDRVAW |
| 96 | | HQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFHLWYI |
| 97 | | HWVRQAPGQGLEWMGRISPGSGNVKYNEKFKGR |
| | | VTITADTSTSTAYMELSSLRSEDTAVYYCAGSYYS |
| | | YDVLDYWGQGTTVTVSS |

A-H.31

| | | |
|---|---|---|
| SEQ ID NO: | VH + VL | QVQLVQSGAEVKKPGSSVKVSCKASGHDFKLTYI |
| 98 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFKLTYI |
| 33 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSS |

A-H.31

| | | |
|---|---|---|
| SEQ ID NO: | VH + VL | QVQLVQSGAEVKKPGSSVKVSCKASGTDFHLWYI |
| 99 | | HWVRQAPGQGLEWMGRVFAGSGSYRYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFHLWYI |
| 100 | | HWVRQAPGQGLEWMGRVFAGSGSYRYNEKFKGR |
| | | VTITADTSTSTAYMELSSLRSEDTAVYYCAGSYYS |
| | | YDVLDYWGQGTTVTVSS |

A-H.32

| | | |
|---|---|---|
| SEQ ID NO: | VH + VL | QVQLVQSGAEVKKPGSSVKVSCKASGTDFDKIYIH |
| 101 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVADR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVADRVAW |
| 102 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKIYIH |
| 38 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSS |

A-H.33

| | | |
|---|---|---|
| SEQ ID NO: | VH + VL | QVQLVQSGAEVKKPGSSVKVSCKASGTDFKLTYIH |
| 103 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVEDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVEDRVAW |
| 24 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 104 | | WVRQAPGQGLEWMGRISAGSGNTKYNEKFKGRV |
| | | TITADTSTSTAYMELSSLRSEDTAVYYCAGSYYSY |
| | | DVLDYWGQGTTVTVSS |

A-H.34

| | | |
|---|---|---|
| SEQ ID NO: | VH + VL | QVQLVQSGAEVKKPGSSVKVSCKASGTDFRLTYIH |
| 105 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGNRVAW |
| 58 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFRLTYIH |
| 106 | | WVRQAPGQGLEWMGRISPGSGNTKYNEKFKGRV |
| | | TITADTSTSTAYMELSSLRSEDTAVYYCAGSYYSY |
| | | DVLDYWGQGTTVTVSS |

A-H.35

| | | |
|---|---|---|
| SEQ ID NO: | VH + VL | QVQLVQSGAEVKKPGSSVKVSCKASGHDFDKTYI |
| 107 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVE |
| | | DRVAWYQQKPGKAPKALIYSSSHRYKGVPSRFSGS |
| | | GSGTEFTLTISSLQPEDFATYFCQQFKSYPLTFGQG |
| | | TKLEIK |
| SEQ ID NO: | VL | DIQMTQSPSFLSASVGDRVTITCKASQNVEDRVAW |
| 24 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFDKTYI |
| 108 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSS |

A-H.36

| | | |
|---|---|---|
| SEQ ID NO: | VH + VL | QVQLVQSGAEVKKPGSSVKVSCKASGHDFKLTYI |
| 109 | | HWVRQAPGQGLEWMGRVSPGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVED |
| | | RVAWHQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVEDRVAW |
| 110 | | HQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFKLTYI |
| 111 | | HWVRQAPGQGLEWMGRVSPGSGNTKYNEKFKGR |
| | | VTITADTSTSTAYMELSSLRSEDTAVYYCAGSYYS |
| | | YDVLDYWGQGTTVTVSS |

A-H.37

| | | |
|---|---|---|
| SEQ ID NO: | VH + VL | QVQLVQSGAEVKKPGSSVKVSCKASGHDFDKTYI |
| 112 | | HWVRQAPGQGLEWMGRIYPGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVAD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVADRVAW |
| 102 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFDKTYI |
| 113 | | HWVRQAPGQGLEWMGRIYPGSGNVKYNEKFKGR |
| | | VTITADTSTSTAYMELSSLRSEDTAVYYCAGSYYS |
| | | YDVLDYWGQGTTVTVSS |

A-H.38

| | | |
|---|---|---|
| SEQ ID NO: | VH + VL | QVQLVQSGAEVKKPGSSVKVSCKASGTDFDKTYI |
| 114 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKTYI |
| 115 | | HWVRQAPGQGLEWMGRISAGSGNVKYNEKFKGR |
| | | VTITADTSTSTAYMELSSLRSEDTAVYYCAGSYYS |
| | | YDVLDYWGQGTTVTVSS |

A-H.39

| | | |
|---|---|---|
| SEQ ID NO: | VH + VL | QVQLVQSGAEVKKPGSSVKVSCKASGTDFDKIYIH |
| 116 | | WVRQAPGQGLEWMGRISAGSGNIKYNEKFKGRVT |
| | | ITADTSTSTAYMELSSLRSEDTAVYYCAGSYYSYD |
| | | VLDYWGQGTTVTVSSGGGGSGGGGSGGGGSGGG |
| | | GSDIQMTQSPSFLSASVGDRVTITCKASQNVDDRV |
| | | AWYQQKPGKAPKALIYSSSHRYKGVPSRFSGSGSG |
| | | TEFTLTISSLQPEDFATYFCQQFKSYPLTFGQGTKLE |
| | | IK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKIYIH |
| 117 | | WVRQAPGQGLEWMGRISAGSGNIKYNEKFKGRVT |
| | | ITADTSTSTAYMELSSLRSEDTAVYYCAGSYYSYD |
| | | VLDYWGQGTTVTVSS |

A-H.40

| | | |
|---|---|---|
| SEQ ID NO: | VH + VL | QVQLVQSGAEVKKPGSSVKVSCKASGTDFDKIYIH |
| 118 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGDR |
| | | VAWYQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVGDRVAW |
| 55 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFDKIYIH |
| 27 | | WVRQAPGQGLEWMGRISAGSGNVKYNEKFKGRV |
| | | TITADTSTSTAYMELSSLRSEDTAVYYCAGSYYSY |
| | | DVLDYWGQGTTVTVSS |

A-H.41

| | | |
|---|---|---|
| SEQ ID NO: | VH + VL | QVQLVQSGAEVKKPGSSVKVSCKASGGTFKLTYIH |
| 119 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDD |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDDRVAW |
| 29 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGGTFKLTYIH |
| 120 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSS |

A-H.42

| | | |
|---|---|---|
| SEQ ID NO: | VH + VL | QVQLVQSGAEVKKPGSSVKVSCKASGTDFKLTYIH |
| 121 | | WVRQAPGQGLEWMGRISPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNR |
| | | VAWHQQKPGKAPKALIYSSSHRYKGVPSRFSGSGS |
| | | GTEFTLTISSLQPEDFATYFCQQFKSYPLTFGQGTK |
| | | LEIK |
| SEQ ID NO: | VL | DIQMTQSPSFLSASVGDRVTITCKASQNVDNRVAW |
| 122 | | HQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGTDFKLTYIH |
| 123 | | WVRQAPGQGLEWMGRISPGSGNVKYNEKFKGRV |
| | | TITADTSTSTAYMELSSLRSEDTAVYYCAGSYYSY |
| | | DVLDYWGQGTTVTVSS |

A-H.43

| | | |
|---|---|---|
| SEQ ID NO: | VH + VL | QVQLVQSGAEVKKPGSSVKVSCKASGHDFDKFYI |
| 124 | | HWVRQAPGQGLEWMGRVSAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVDN |
| | | RVAWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VL | DIQMTQSPSFLSASVGDRVTITCKASQNVDNRVAW |
| 32 | | YQQKPGKAPKALIYSSSHRYKGVPSRFSGSGSGTEF |
| | | TLTISSLQPEDFATYFCQQFKSYPLTFGQGTKLEIK |
| SEQ ID NO: | VH | QVQLVQSGAEVKKPGSSVKVSCKASGHDFDKFYI |
| 125 | | HWVRQAPGQGLEWMGRVSAGSGNTKYNEKFKGR |
| | | VTITADTSTSTAYMELSSLRSEDTAVYYCAGSYYS |
| | | YDVLDYWGQGTTVTVSS |

A-H.44

| | | |
|---|---|---|
| SEQ ID NO: | VH + VL | QVQLVQSGAEVKKPGSSVKVSCKASGTDFDKFYIH |
| 126 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGD |
| | | RVVWYQQKPGKAPKALIYSSSHRYKGVPSRFSGSG |
| | | SGTEFTLTISSLQPEDFATYFCQQFKSYPLTFGQGT |
| | | KLEIK |
| SEQ ID NO: | VH | QVQLVQSGAEVKKPGSSVKVSCKASGTDFDKFYIH |
| 127 | | WVRQAPGQGLEWMGRVSAGSGNVKYNEKFKGR |
| | | VTITADTSTSTAYMELSSLRSEDTAVYYCAGSYYS |
| | | YDVLDYWGQGTTVTVSS |

A-H.45

| | | |
|---|---|---|
| SEQ ID NO: | VH + VL | QVQLVQSGAEVKKPGSSVKVSCKASGYSFTTYYIH |
| 128 | | WVRQAPGQGLEWMGWFSAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVGIN |
| | | VVWHQQKPGKAPKALIYSSSHRYSGVPSRFSGSGS |
| | | GTEFTLTISSLQPEDFATYFCQQFKSYPLTFGQGTK |
| | | LEIK |
| SEQ ID NO: | VH | QVQLVQSGAEVKKPGSSVKVSCKASGYSFTTYYIH |
| 129 | | WVRQAPGQGLEWMGWFSAGSGNTKYNEKFKGR |
| | | VTITADTSTSTAYMELSSLRSEDTAVYYCAVSYYS |
| | | YDVLDYWGQGTTVTVSS |

A-H.46

| | | |
|---|---|---|
| SEQ ID NO: | VH + VL | QVQLVQSGAEVKKPGSSVKVSCKASGYSFTTYYIH |
| 130 | | WVRQAPGQGLEWMGWFSAGSGNTKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGIN |
| | | VVWHQQKPGKAPKALIYSSSHRYSGVPSRFSGSGS |
| | | GTEFTLTISSLQPEDFATYFCQQFKSYPLTFGQGTK |
| | | LEIK |
| SEQ ID NO: | VH | QVQLVQSGAEVKKPGSSVKVSCKASGYSFTTYYIH |
| 131 | | WVRQAPGQGLEWMGWFSAGSGNTKYNEKFKGR |
| | | VTITADTSTSTAYMELSSLRSEDTAVYYCAGSYYS |
| | | YDVLDYWGQGTTVTVSS |

A-H.47

| | | |
|---|---|---|
| SEQ ID NO: | VH + VL | QVQLVQSGAEVKKPGSSVKVSCKASGYSFTTYYIH |
| 132 | | WVRQAPGQGLEWMGWFFPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKLE |
| | | IK |
| SEQ ID NO: | VH | QVQLVQSGAEVKKPGSSVKVSCKASGYSFTTYYIH |
| 133 | | WVRQAPGQGLEWMGWFFPGSGNTKYNEKFKGRV |
| | | TITADTSTSTAYMELSSLRSEDTAVYYCAGSYYSY |
| | | DVLDYWGQGTTVTVSS |

A-H.48

| | | |
|---|---|---|
| SEQ ID NO: | VH + VL | QVQLVQSGAEVKKPGSSVKVSCKASGYSFTTYYIH |
| 134 | | WVRQAPGQGLEWMGWFSPGSGNTKYNEKFKGRV |
| | | TITADTSTSTAYMELSSLRSEDTAVYYCAVSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKLE |
| | | IK |
| SEQ ID NO: | VH | QVQLVQSGAEVKKPGSSVKVSCKASGYSFTTYYIH |
| 135 | | WVRQAPGQGLEWMGWFSPGSGNTKYNEKFKGRV |
| | | TITADTSTSTAYMELSSLRSEDTAVYYCAVSYYSY |
| | | DVLDYWGQGTTVTVSS |

A-H.49

| | | |
|---|---|---|
| SEQ ID NO: | VH + VL | QVQLVQSGAEVKKPGSSVKVSCKASGYSFTTYYIH |
| 136 | | WVRQAPGQGLEWMGWFSPGSGNTKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKLE |
| | | IK |
| SEQ ID NO: | VH | QVQLVQSGAEVKKPGSSVKVSCKASGYSFTTYYIH |
| 137 | | WVRQAPGQGLEWMGWFSPGSGNTKYNEKFKGRV |
| | | TITADTSTSTAYMELSSLRSEDTAVYYCAGSYYSY |
| | | DVLDYWGQGTTVTVSS |

A-H.50

| | | |
|---|---|---|
| SEQ ID NO: | VH + VL | QVQLVQSGAEVKKPGSSVKVSCKASGYSFTTYYIH |
| 138 | | WVRQAPGQGLEWMGRIFPGSGNIKYNEKFKGRVT |
| | | ITADTSTSTAYMELSSLRSEDTAVYYCAGSYYSYD |
| | | VLDYWGQGTTVTVSSGGGGSGGGGSGGGGSGGG |
| | | GSDIQMTQSPSFLSASVGDRVTITCKASQNVGINVV |
| | | WHQQKPGKAPKALIYSSSHRYSGVPSRFSGSGSGT |
| | | EFTLTISSLQPEDFATYFCQQFKSYPLTFGQGTKLEI |
| | | K. |
| SEQ ID NO: | VH | QVQLVQSGAEVKKPGSSVKVSCKASGYSFTTYYIH |
| 139 | | WVRQAPGQGLEWMGRIFPGSGNIKYNEKFKGRVT |
| | | ITADTSTSTAYMELSSLRSEDTAVYYCAGSYYSYD |
| | | VLDYWGQGTTVTVSS |

A-H.51

| | | |
|---|---|---|
| SEQ ID NO: | VH + VL | QVQLVQSGAEVKKPGSSVKVSCKASGYSFTTYYIH |
| 140 | | WVRQAPGQGLEWMGWFFPGSGNIKYNEKFKGRV |
| | | TITADTSTSTAYMELSSLRSEDTAVYYCAGSIYSAG |
| | | VLDYWGQGTTVTVSSGGGGSGGGGSGGGGSGGG |
| | | GSDIQMTQSPSFLSASVGDRVTITCKASQNVGINVV |
| | | WHQQKPGKAPKALIYSSSHRYSGVPSRFSGSGSGT |
| | | EFTLTISSLQPEDFATYFCQQFKSYPLTFGQGTKLEI |
| | | K |
| SEQ ID NO: | VH | QVQLVQSGAEVKKPGSSVKVSCKASGYSFTTYYIH |
| 141 | | WVRQAPGQGLEWMGWFFPGSGNIKYNEKFKGRV |
| | | TITADTSTSTAYMELSSLRSEDTAVYYCAGSIYSAG |
| | | VLDYWGQGTTVTVSS |

A-H.52

| | | |
|---|---|---|
| SEQ ID NO: | VH + VL | QVQLVQSGAEVKKPGSSVKVSCKASGYSFTLGYIH |
| 142 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKLE |
| | | IK |
| SEQ ID NO: | VH | QVQLVQSGAEVKKPGSSVKVSCKASGYSFTLGYIH |
| 143 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |

A-H.53

| | | |
|---|---|---|
| SEQ ID NO: | VH + VL | QVQLVQSGAEVKKPGSSVKVSCKASGYSFRLTYIH |
| 144 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVGINV |
| | | VWHQQKPGKAPKALIYSSSHRYSGVPSRFSGSGSG |
| | | TEFTLTISSLQPEDFATYFCQQFKSYPLTFGQGTKLE |
| | | IK |
| SEQ ID NO: | VH | QVQLVQSGAEVKKPGSSVKVSCKASGYSFRLTYIH |
| 145 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |

A-H.54

| | | |
|---|---|---|
| SEQ ID NO: | VH + VL | QVQLVQSGAEVKKPGSSVKVSCKASGYSFHNWYI |
| 146 | | HWVRQAPGQGLEWMGWFFPGSGNIKYNEKFKGR |
| | | VTITADTSTSTAYMELSSLRSEDTAVYYCAGSYYS |
| | | YDVLDYWGQGTTVTVSSGGGGSGGGGSGGGGSG |
| | | GGGSDIQMTQSPSFLSASVGDRVTITCKASQNVGIN |
| | | VVWHQQKPGKAPKALIYSSSHRYSGVPSRFSGSGS |
| | | GTEFTLTISSLQPEDFATYFCQQFKSYPLTFGQGTK |
| | | LEIK |
| SEQ ID NO: | VH | QVQLVQSGAEVKKPGSSVKVSCKASGYSFHNWYI |
| 147 | | HWVRQAPGQGLEWMGWFFPGSGNIKYNEKFKGR |
| | | VTITADTSTSTAYMELSSLRSEDTAVYYCAGSYYS |
| | | YDVLDYWGQGTTVTVSS |

A-H.55 antibody

| | | |
|---|---|---|
| SEQ ID NO: | HC CDR1 | GYSFTTYYIH |
| 3 | (Combined) | |
| SEQ ID NO: | HC CDR2 | WFFPGSGNIKYNEKFKG |
| 4 | (Combined) | |
| SEQ ID NO: | HC CDR3 | SYYSYDVLDY |
| 5 | (Combined) | |
| SEQ ID NO: | HC CDR1 (Kabat) | TYYIH |
| 45 | | |
| SEQ ID NO: | HC CDR2 (Kabat) | WFFPGSGNIKYNEKFKG |
| 4 | | |
| SEQ ID NO: | HC CDR3 (Kabat) | SYYSYDVLDY |
| 5 | | |
| SEQ ID NO: | HC CDR1 | GYSFTTY |
| 15 | (Chothia) | |
| SEQ ID NO: | HC CDR2 | FPGSGN |
| 16 | (Chothia) | |
| SEQ ID NO: | HC CDR3 | SYYSYDVLDY |
| 5 | (Chothia) | |
| SEQ ID NO: | VH | QVQLVQSGAEVKKPGSSVKVSCKASGYSFTTYYIH |
| 9 | | WVRQAPGQGLEWMGWFFPGSGNIKYNEKFKGRV |
| | | TITADTSTSTAYMELSSLRSEDTAVYYCAGSYYSY |
| | | DVLDYWGQGTTVTVSS |
| SEQ ID NO: | LC CDR1 | KASQNVGINVV |
| 6 | (Combined) | |
| SEQ ID NO: | LC CDR2 | SSSHRYS |
| 7 | (Combined) | |
| SEQ ID NO: | LC CDR3 | QQFKSYPLT |
| 8 | (Combined) | |
| SEQ ID NO: | LC CDR1 (Kabat) | KASQNVGINVV |
| 6 | | |
| SEQ ID NO: | LC CDR2 (Kabat) | SSSHRYS |
| 7 | | |
| SEQ ID NO: | LC CDR3 (Kabat) | QQFKSYPLT |
| 8 | | |
| SEQ ID NO: | LC CDR1 | KASQNVGINVV |
| 6 | (Chothia) | |
| SEQ ID NO: | LC CDR2 (chothia) | SSSHRYS |
| 7 | | |
| SEQ ID NO: | LC CDR3 (chothia) | QQFKSYPLT |
| 8 | | |
| SEQ ID NO: | VL | QSVLTQPPSVSEAPRQRVTISCKASQNVGINVVWH |
| 148 | | QQLPGKAPKALIYSSSHRYSGVSDRFSGSGSGTSFS |
| | | LAISGLQSEDEADYFCQQFKSYPLTFGTGTKVTVL |

A-H.56

| | | |
|---|---|---|
| SEQ ID NO: | VH + VL (ScFv) | QVQLVQSGAEVKKPGSSVKVSCKASGHDFDKFYI |
| 149 | | HWVRQAPGQGLEWMGRVSAGSGNVKYNEKFKG |
| | | RVTITADTSTSTAYMELSSLRSEDTAVYYCAGSYY |
| | | SYDVLDYWGQGTTVTVSSGGGGSGGGGSGGGGS |
| | | GGGGSDIQMTQSPSFLSASVGDRVTITCKASQNVG |
| | | NRVAWYQQKPGKAPKALIYSSSHRYKGVPSRFSGS |
| | | GSGTEFTLTISSLQPEDFATYFCQQFKSYPLTFGQG |
| | | TKLEIK |

A-H.57

| | | |
|---|---|---|
| SEQ ID NO: | VH + VL (ScFv) | QVQLVQSGAEVKKPGSSVKVSCKASGHDFRLTYIH |
| 150 | | WVRQAPGQGLEWMGRVSAGSGNTKYNEKFKGRV |
| | | TITADTSTSTAYMELSSLRSEDTAVYYCAVSYYSY |
| | | DVLDYWGQGTTVTVSSGGGGSGGGGSGGGGSGG |
| | | GGSDIQMTQSPSFLSASVGDRVTITCKASQNVGDR |
| | | VVWHQQKPGKAPKALIYSSSHRYKGVPSRFSGSGS |
| | | GTEFTLTISSLQPEDFATYFCQQFKSYPLTFGQGTK |
| | | LEIK |

A-H.58

| | | |
|---|---|---|
| SEQ ID NO: | VH + VL (ScFv) | QVQLVQSGAEVKKPGSSVKVSCKASGHDFRLTYIH |
| 151 | | WVRQAPGQGLEWMGRVSAGSGNVKYNEKFKGR |
| | | VTITADTSTSTAYMELSSLRSEDTAVYYCAVSYYS |
| | | YDVLDYWGQGTTVTVSSGGGGSGGGGGGGGSG |
| | | GGGSDIQMTQSPSFLSASVGDRVTITCKASQNVGN |
| | | RVVWHQQKPGKAPKALIYSSSHRYKGVPSRFSGSG |
| | | SGTEFTLTISSLQPEDFATYFCQQFKSYPLTFGQGT |
| | | KLEIK |

A-H.59

| | | |
|---|---|---|
| SEQ ID NO: | VH + VL (ScFv) | QVQLVQSGAEVKKPGSSVKVSCKASGHDFRLTYIH |
| 152 | | WVRQAPGQGLEWMGRIYAGSGNVKYNEKFKGRV |
| | | TITADTSTSTAYMELSSLRSEDTAVYYCAVSYYSY |
| | | DVLDYWGQGTTVTVSSGGGGSGGGGSGGGGSGG |
| | | GGSDIQMTQSPSFLSASVGDRVTITCKASQNVADR |
| | | VVWHQQKPGKAPKALIYSSSHRYKGVPSRFSGSGS |
| | | GTEFTLTISSLQPEDFATYFCQQFKSYPLTFGQGTK |
| | | LEIK |

A-H.60

| | | |
|---|---|---|
| SEQ ID NO: | VH + VL (ScFv) | QVQLVQSGAEVKKPGSSVKVSCKASGHDFKLTYI |
| 153 | | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVGD |
| | | RVAWHQQKPGKAPKALIYSSSHRYKGVPSRFSGSG |
| | | SGTEFTLTISSLQPEDFATYFCQQFKSYPLTFGQGT |
| | | KLEIK |

A-H.61

| | | |
|---|---|---|
| SEQ ID NO: | VH + VL (ScFv) | QVQLVQSGAEVKKPGSSVKVSCKASGHDFKLTYI |
| 154 | | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVDN |
| | | RVAWHQQKPGKAPKALIYSSSHRYKGVPSRFSGSG |
| | | SGTEFTLTISSLQPEDFATYFCQQFKSYPLTFGQGT |
| | | KLEIK |

A-H.62

| | | |
|---|---|---|
| SEQ ID NO: | VH + VL (ScFv) | QVQLVQSGAEVKKPGSSVKVSCKASGHDFRLTYIH |
| 155 | | WVRQAPGQGLEWMGRVSAGSGNVKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVAD |
| | | RVVWHQQKPGKAPKALIYSSSHRYKGVPSRFSGSG |
| | | SGTEFTLTISSLQPEDFATYFCQQFKSYPLTFGQGT |
| | | KLEIK |

A-H.63

| | | |
|---|---|---|
| SEQ ID NO: | VH + VL (ScFv) | QVQLVQSGAEVKKPGSSVKVSCKASGHDFRLTYIH |
| 156 | | WVRQAPGQGLEWMGRVYAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVED |
| | | RVVWHQQKPGKAPKALIYSSSHRYKGVPSRFSGSG |
| | | SGTEFTLTISSLQPEDFATYFCQQFKSYPLTFGQGT |
| | | KLEIK |

A-H.64

| | | |
|---|---|---|
| SEQ ID NO: | VH + VL (ScFv) | QVQLVQSGAEVKKPGSSVKVSCKASGHDFKLTYI |
| 157 | | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVAD |
| | | RVVWHQQKPGKAPKALIYSSSHRYKGVPSRFSGSG |
| | | SGTEFTLTISSLQPEDFATYFCQQFKSYPLTFGQGT |
| | | KLEIK |

A-H.65

| | | |
|---|---|---|
| SEQ ID NO: | VH + VL (ScFv) | QVQLVQSGAEVKKPGSSVKVSCKASGHDFKLTYI |
| 158 | | HWVRQAPGQGLEWMGRISAGSGNTKYNEKFKGR |
| | | VTITADTSTSTAYMELSSLRSEDTAVYYCAVSYYS |
| | | YDVLDYWGQGTTVTVSSGGGGSGGGGGGGGSG |
| | | GGGSDIQMTQSPSFLSASVGDRVTITCKASQNVGD |
| | | RVVWHQQKPGKAPKALIYSSSHRYKGVPSRFSGSG |
| | | SGTEFTLTISSLQPEDFATYFCQQFKSYPLTFGQGT |
| | | KLEIK |

A-H.66

| | | |
|---|---|---|
| SEQ ID NO: | VH + VL (ScFv) | QVQLVQSGAEVKKPGSSVKVSCKASGHDFKLTYI |
| 159 | | HWVRQAPGQGLEWMGRIYAGSGNTKYNEKFKGR |
| | | VTITADTSTSTAYMELSSLRSEDTAVYYCAVSYYS |
| | | YDVLDYWGQGTTVTVSSGGGGSGGGGSGGGGSG |
| | | GGGSDIQMTQSPSFLSASVGDRVTITCKASQNVGD |
| | | RVVWHQQKPGKAPKALIYSSSHRYKGVPSRFSGSG |
| | | SGTEFTLTISSLQPEDFATYFCQQFKSYPLTFGQGT |
| | | KLEIK |

A-H.67

| | | |
|---|---|---|
| SEQ ID NO: | VH + VL (ScFv) | QVQLVQSGAEVKKPGSSVKVSCKASGTDFKLTYIH |
| 160 | | WVRQAPGQGLEWMGRIFPGSGNVKYNEKFKGRV |
| | | TITADTSTSTAYMELSSLRSEDTAVYYCAVSYYSY |
| | | DVLDYWGQGTTVTVSSGGGGSGGGGSGGGGSGG |
| | | GGSDIQMTQSPSFLSASVGDRVTITCKASQNVDNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |

A-H.68

| | | |
|---|---|---|
| SEQ ID NO: | VH + VL (ScFv) | QVQLVQSGAEVKKPGSSVKVSCKASGHDFRLTYIH |
| 161 | | WVRQAPGQGLEWMGRISAGSGNVKYNEKFKGRV |
| | | TITADTSTSTAYMELSSLRSEDTAVYYCAVSYYSY |
| | | DVLDYWGQGTTVTVSSGGGGSGGGGSGGGGSGG |
| | | GGSDIQMTQSPSFLSASVGDRVTITCKASQNVADR |
| | | VAWYQQKPGKAPKALIYSSSHRYKGVPSRFSGSGS |
| | | GTEFTLTISSLQPEDFATYFCQQFKSYPLTFGQGTK |
| | | LEIK |

A-H.69 (also referred to as A-H.13)

| | | |
|---|---|---|
| SEQ ID NO: | VH + VL (ScFv) | QVQLVQSGAEVKKPGSSVKVSCKASGTDFKLTYIH |
| 60 | | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSSGGGGSGGGGSGGGGSGG |
| | | GGSDIQMTQSPSFLSASVGDRVTITCKASQNVDNR |
| | | VAWYQQKPGKAPKALIYSSSHRYKGVPSRFSGSGS |
| | | GTEFTLTISSLQPEDFATYFCQQFKSYPLTFGQGTK |
| | | LEIK |

A-H humanized-matured VH

| | | |
|---|---|---|
| SEQ ID NO: | VH-humanized | QVQLVQSGAEVKKPGSSVKVSCKASGTDFKLTYIH |
| 36 | matured 1 | WVRQAPGQGLEWMGRIFPGSGNVKYNEKFKGRV |
| | | TITADTSTSTAYMELSSLRSEDTAVYYCAGSYYSY |
| | | DVLDYWGQGTTVTVSS |
| SEQ ID NO: | VH-humanized | QVQLVQSGAEVKKPGSSVKVSCKASGTDFKLTYIH |
| 162 | matured 2 | WVRQAPGQGLEWMGRIFPGSGNVKYNEKFKGRV |
| | | TITADTSTSTAYMELSSLRSEDTAVYYCAVSYYSY |
| | | DVLDYWGQGTTVTVSS |
| SEQ ID NO: | VH-humanized | QVQLVQSGAEVKKPGSSVKVSCKASGHDFRLTYIH |
| 163 | matured 3 | WVRQAPGQGLEWMGRISAGSGNVKYNEKFKGRV |
| | | TITADTSTSTAYMELSSLRSEDTAVYYCAVSYYSY |
| | | DVLDYWGQGTTVTVSS |

A-H humanized-matured VL

| | | |
|---|---|---|
| SEQ ID NO: | VL-humanized | DIQMTQSPSFLSASVGDRVTITCKASQNVDNRVAW |
| 32 | matured 1 | YQQKPGKAPKALIYSSSHRYKGVPSRFSGSGSGTEF |
| | | TLTISSLQPEDFATYFCQQFKSYPLTFGQGTKLEIK |
| SEQ ID NO: | VL-humanized | DIQMTQSPSFLSASVGDRVTITCKASQNVADRVAW |
| 102 | matured 2 | YQQKPGKAPKALIYSSSHRYKGVPSRFSGSGSGTEF |
| | | TLTISSLQPEDFATYFCQQFKSYPLTFGQGTKLEIK |

A-H.70

| | | |
|---|---|---|
| SEQ ID NO: | HC CDR1 | GHDFRLTYIH |
| 164 | | |
| SEQ ID NO: | HC CDR2 | RVSAGSGNVKYNEKFKG |
| 165 | | |
| SEQ ID NO: | HC CDR3 | SYYSYDVLDY |
| 5 | | |
| SEQ ID NO: | LC CDR1 | KASQNVGNRVV |
| 166 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFRLTYIH |
| 168 | (CDRs underlined) | WVRQAPGQGLEWMGRVSAGSGNVKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVGNRVVW |
| 169 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.71

| | | |
|---|---|---|
| SEQ ID NO: | HC CDR1 | GHDFRLTYIH |
| 164 | | |
| SEQ ID NO: | HC CDR2 | RIYAGSGNVKYNEKFKG |
| 170 | | |
| SEQ ID NO: | HC CDR3 | SYYSYDVLDY |
| 5 | | |
| SEQ ID NO: | LC CDR1 | KASQNVADRVV |
| 171 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFRLTYIH |
| 172 | (CDRs underlined) | WVRQAPGQGLEWMGRIYAGSGNVKYNEKFKGRV |
| | | TITADTSTSTAYMELSSLRSEDTAVYYCAVSYYSY |
| | | DVLDYWGQGTTVTVSS |
| SEQ ID NO: | VL | DIQMTQSPSFLSASVGDRVTITCKASQNVADRVVW |
| 173 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.72

| | | |
|---|---|---|
| SEQ ID NO: | HC CDR1 | GHDFKLTYIH |
| 174 | | |
| SEQ ID NO: | HC CDR2 | RVSAGSGNTKYNEKFKG |
| 175 | | |
| SEQ ID NO: | HC CDR3 | SYYSYDVLDY |
| 5 | | |
| SEQ ID NO: | LC CDR1 | KASQNVGDRVA |
| 364 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 177 | (CDRs underlined) | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVGDRVAW |
| 96 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.73

| | | |
|---|---|---|
| SEQ ID NO: | HC CDR1 | GHDFKLTYIH |
| 174 | | |
| SEQ ID NO: | HC CDR2 | RVSAGSGNTKYNEKFKG |
| 175 | | |
| SEQ ID NO: | HC CDR3 | SYYSYDVLDY |
| 5 | | |
| SEQ ID NO: | LC CDR1 | KASQNVDNRVA |
| 176 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 177 | (CDRs underlined) | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVDNRVAW |
| 122 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.74

| | | |
|---|---|---|
| SEQ ID NO: | HC CDR1 | GHDFRLTYIH |
| 164 | | |
| SEQ ID NO: | HC CDR2 | RVSAGSGNVKYNEKFKG |
| 165 | | |
| SEQ ID NO: | HC CDR3 | SYYSYDVLDY |
| 5 | | |
| SEQ ID NO: | LC CDR1 | KASQNVADRVV |
| 171 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFRLTYIH |
| 168 | (CDRs underlined) | WVRQAPGQGLEWMGRVSAGSGNVKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVADRVVW |
| 173 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.75

| | | |
|---|---|---|
| SEQ ID NO: | HC CDR1 | GHDFRLTYIH |
| 164 | | |
| SEQ ID NO: | HC CDR2 | RVYAGSGNTKYNEKFKG |
| 178 | | |
| SEQ ID NO: | HC CDR3 | SYYSYDVLDY |
| 5 | | |
| SEQ ID NO: | LC CDR1 | KASQNVEDRVV |
| 179 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFRLTYIH |
| 180 | (CDRs underlined) | WVRQAPGQGLEWMGRVYAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVEDRVVW |
| 180 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.76

| | | |
|---|---|---|
| SEQ ID NO: | HC CDR1 | GHDFKLTYIH |
| 174 | | |
| SEQ ID NO: | HC CDR2 | RVSAGSGNTKYNEKFKG |
| 175 | | |
| SEQ ID NO: | HC CDR3 | SYYSYDVLDY |
| 5 | | |
| SEQ ID NO: | LC CDR1 | KASQNVADRVV |
| 171 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 177 | (CDRs underlined) | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVADRVVW |
| 173 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.77

| | | |
|---|---|---|
| SEQ ID NO: | HC CDR1 | GHDFKLTYIH |
| 174 | | |
| SEQ ID NO: | HC CDR2 | RISAGSGNTKYNEKFKG |
| 181 | | |
| SEQ ID NO: | HC CDR3 | SYYSYDVLDY |
| 5 | | |
| SEQ ID NO: | LC CDR1 | KASQNVGDRVV |
| 182 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 183 | (CDRs underlined) | HWVRQAPGQGLEWMGRISAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVGDRVVW |
| 184 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.78

| | | |
|---|---|---|
| SEQ ID NO: | HC CDR1 | GHDFKLTYIH |
| 174 | | |
| SEQ ID NO: | HC CDR2 | RIYAGSGNTKYNEKFKG |
| 365 | | |
| SEQ ID NO: | HC CDR3 | SYYSYDVLDY |
| 5 | | |
| SEQ ID NO: | LC CDR1 | KASQNVGDRVV |
| 182 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 185 | (CDRs underlined) | HWVRQAPGQGLEWMGRIYAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVGDRVVW |
| 184 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.79

| | | |
|---|---|---|
| SEQ ID NO: | HC CDR1 | GHDFKLTYIH |
| 174 | | |
| SEQ ID NO: | HC CDR2 | RVSAGSGNTKYNEKFKG |
| 175 | | |
| SEQ ID NO: | HC CDR3 | SYYSYDVLDY |
| 5 | | |
| SEQ ID NO: | LC CDR1 | RASQNVDNRLG |
| 186 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 177 | (CDRs underlined) | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCRASQNVDNRLGW |
| 187 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.80

| | | |
|---|---|---|
| SEQ ID NO: | HC CDR1 | GHDFKLTYIH |
| 174 | | |
| SEQ ID NO: | HC CDR2 | RVSAGSGNTKYNEKFKG |
| 175 | | |
| SEQ ID NO: | HC CDR3 | SYYSYDVLDY |
| 5 | | |
| SEQ ID NO: | LC CDR1 | KASQNVDNRVA |
| 176 | | |
| SEQ ID NO: | LC CDR2 | AASSLQK |
| 188 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 177 | (CDRs underlined) | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVDNRVAW |
| 189 | (CDRs underlined) | HQQKPGKAPKALIYAASSLQKGVPSRFSGSGSGTE |
| | | FTLTISSLQPEDFATYFCQQFKSYPLTFGQGTKLEIK |

A-H.81

| | | |
|---|---|---|
| SEQ ID NO: | HC CDR1 | GHDFKLTYIH |
| 174 | | |
| SEQ ID NO: | HC CDR2 | RVSAGSGNTKYNEKFKG |
| 175 | | |
| SEQ ID NO: | HC CDR3 | SYYSYDVLDY |
| 5 | | |
| SEQ ID NO: | LC CDR1 | KASQNVDNRVA |
| 176 | | |
| SEQ ID NO: | LC CDR2 | SSSHRYK |
| 167 | | |
| SEQ ID NO: | LC CDR3 | LQHNSYPLT |
| 190 | | |
| SEQ ID NO: | VH | QVQLVQSGAEVKKPGSSVKVSCKASGHDFKLTYI |
| 177 | (CDRs underlined) | HWVRQAPGQGLEWMGRVSAGSGNTKYNEKFKGR |
| | | VTITADTSTSTAYMELSSLRSEDTAVYYCAVSYYS |
| | | YDVLDYWGQGTTVTVSS |
| SEQ ID NO: | VL | DIQMTQSPSFLSASVGDRVTITCKASQNVDNRVAW |
| 191 | (CDRs underlined) | HQQKPGKAPKALIYSSSHRYKGVPSRFSGSGSGTEF |
| | | TLTISSLQPEDFATYFCLQHNSYPLTFGQGTKLEIK |

A-H.82

| | | |
|---|---|---|
| SEQ ID NO: | HC CDR1 | GHDFKLTYIH |
| 174 | | |
| SEQ ID NO: | HC CDR2 | RVSAGSGNVNYAQKFQG |
| 192 | | |
| SEQ ID NO: | HC CDR3 | SYYSYDVLDY |
| 5 | | |
| SEQ ID NO: | LC CDR1 | RASQNVDNRLG |
| 186 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 193 | (CDRs underlined) | HWVRQAPGQGLEWMGRVSAGSGNVNYAQKFQG |
| | | RVTITADTSTSTAYMELSSLRSEDTAVYYCAVSYY |
| | | SYDVLDYWGQGTTVTVSS |
| SEQ ID NO: | VL | DIQMTQSPSFLSASVGDRVTITCRASQNVDNRLGW |
| 187 | (CDRs underlined) | HQQKPGKAPKALIYSSSHRYKGVPSRFSGSGSGTEF |
| | | TLTISSLQPEDFATYFCQQFKSYPLTFGQGTKLEIK |

A-H.83

| | | |
|---|---|---|
| SEQ ID NO: | HC CDR1 | GHDFKLTYIH |
| 174 | | |
| SEQ ID NO: | HC CDR2 | RVSAGSGNVNYAQKFQG |
| 192 | | |
| SEQ ID NO: | HC CDR3 | SYYSYDVLDY |
| 5 | | |
| SEQ ID NO: | LC CDR1 | KASQNVDNRVA |
| 176 | | |
| SEQ ID NO: | LC CDR2 | AASSLQK |
| 188 | | |
| SEQ ID NO: | LC CDR3 | QQFKSYPLT |
| 8 | | |
| SEQ ID NO: | VH | QVQLVQSGAEVKKPGSSVKVSCKASGHDFKLTYI |
| 193 | (CDRs underlined) | HWVRQAPGQGLEWMGRVSAGSGNVNYAQKFQG |
| | | RVTITADTSTSTAYMELSSLRSEDTAVYYCAVSYY |
| | | SYDVLDYWGQGTTVTVSS |
| SEQ ID NO: | VL | DIQMTQSPSFLSASVGDRVTITCKASQNVDNRVAW |
| 189 | (CDRs underlined) | HQQKPGKAPKALIYAASSLQKGVPSRFSGSGSGTE |
| | | FTLTISSLQPEDFATYFCQQFKSYPLTFGQGTKLEIK |

A-H.84

| | | |
|---|---|---|
| SEQ ID NO: | HC CDR1 | GHDFKLTYIH |
| 174 | | |
| SEQ ID NO: | HC CDR2 | RVSAGSGNVNYAQKFQG |
| 192 | | |
| SEQ ID NO: | HC CDR3 | SYYSYDVLDY |
| 5 | | |
| SEQ ID NO: | LC CDR1 | KASQNVDNRVA |
| 176 | | |
| SEQ ID NO: | LC CDR2 | SSSHRYK |
| 167 | | |
| SEQ ID NO: | LC CDR3 | LQHNSYPLT |
| 190 | | |
| SEQ ID NO: | VH | QVQLVQSGAEVKKPGSSVKVSCKASGHDFKLTYI |
| 193 | (CDRs underlined) | HWVRQAPGQGLEWMGRVSAGSGNVNYAQKFQG |
| | | RVTITADTSTSTAYMELSSLRSEDTAVYYCAVSYY |
| | | SYDVLDYWGQGTTVTVSS |
| SEQ ID NO: | VL | DIQMTQSPSFLSASVGDRVTITCKASQNVDNRVAW |
| 191 | (CDRs underlined) | HQQKPGKAPKALIYSSSHRYKGVPSRFSGSGSGTEF |
| | | TLTISSLQPEDFATYFCLQHNSYPLTFGQGTKLEIK |

A-H.85

| | | |
|---|---|---|
| SEQ ID NO: | HC CDR1 | GHDFRLTYIH |
| 164 | | |
| SEQ ID NO: | HC CDR2 | RVSAGSGNTKYNEKFKG |
| 175 | | |
| SEQ ID NO: | HC CDR3 | SYYSYDVLDY |
| 5 | | |
| SEQ ID NO: | LC CDR1 | KASQNVGDRVV |
| 182 | | |
| SEQ ID NO: | LC CDR2 | SSSHRYK |
| 167 | | |
| SEQ ID NO: | LC CDR3 | QQFKSYPLT |
| 8 | | |
| SEQ ID NO: | VH (CDRs | QVQLVQSGAEVKKPGSSVKVSCKASGHDFRLTYIH |
| 194 | underlined) | WVRQAPGQGLEWMGRVSAGSGNTKYNEKFKGRV |
| | | TITADTSTSTAYMELSSLRSEDTAVYYCAVSYYSY |
| | | DVLDYWGQGTTVTVSS |
| SEQ ID NO: | VL | DIQMTQSPSFLSASVGDRVTITCKASQNVGDRVVW |
| 184 | (CDRs underlined) | HQQKPGKAPKALIYSSSHRYKGVPSRFSGSGSGTEF |
| | | TLTISSLQPEDFATYFCQQFKSYPLTFGQGTKLEIK |

TABLE 2
Exemplary multispecific molecule that comprise a first domain that binds to a first
target molecule, wherein the exemplary target molecule comprises TCRVα 1 (e.g., TCRVα 1-2).
Exemplary anti-TCRVα 1 antibody includes, but are not limited to, BMX001, which binds to
human TCRVα 1 (e.g., TCRVα 1-2). CDRs are underlined.
BMX001
Antibody binding to human TCRVα 1-2

| | | |
|---|---|---|
| SEQ ID NO: 195 | VH | QVQLVQSGAEVKKPGSSVKVSCKASGFNIKDTHWVRQ |
| | | APGQGLEWMGTDPASGDTRVTITADTSTSTAYMELSSL |
| | | RSEDTAVYYCAVCAHYYRDDVNYAMDYWGQGTTVT |
| | | VSS |
| SEQ ID NO: 196 | VL | DIQMTQSPSFLSASVGDRVTITCQNVGSNWHQQKPGKA |
| | | PKALIYSSSGVPSRFSGSGSGTEFTLTISSLQPEDFATYFC |
| | | QQYNTYPYTFGQGTKLEIK |
| SEQ ID NO: 197 | HC CDR1 | GFNIKDTH |
| SEQ ID NO: 198 | HC CDR2 | TDPASGDT |
| SEQ ID NO: 199 | HC CDR3 | CAHYYRDDVNYAMDY |
| SEQ ID NO: 200 | LC CDR1 | QNVGSN |
| SEQ ID NO: 201 | LC CDR1 | SSS |
| SEQ ID NO: 202 | LC CDR1 | QQYNTYPYT |

TABLE 3
Constant region amino acid sequences of human IgG
heavy chains and human kappa light chain

| | | |
|---|---|---|
| Human kappa | LC | RTVAAPSVFI FPPSDEQLKS GTASVVCLLN NFYPREAKVQ |
| constant region | | WKVDNALQSG NSQESVTEQD SKDSTYSLSS TLTLSKADYE |
| SEQ ID NO: 39 | | KHKVYACEVT HQGLSSPVTK SFNRGEC |
| Human | LC | RTVAAPSVFIFPPSDEQLKSGTASVVCLLNNFYPREAKVQWKVDNALQ |
| Immunoglobulin | | SGNSQESVTEQDSKDSTYSLSSTLTLSKADYEKHKVYACEVTHQGLSSP |
| kappa constant | | VTKSFNRGEC |
| C region | | |
| SEQ ID NO: | | |
| 203 | | |
| IgG4 (S228P) | HC | ASTKGPSVFPLAPCSRSTSESTAALGCLVKDYFPEPVTVSWNSGALTSG |
| mutant constant | | VHTFPAVLQSSGLYSLSSVVTVPSSSLGTKTYTCNVDHKPSNTKVDKR |
| region (EU | | VESKYGPPCPPCPAPEFLGGPSVFLFPPKPKDTLMISRTPEVTCVVVDVS |
| Numbering) | | QEDPEVQFNWYVDGVEVHNAKTKPREEQFNSTYRVVSVLTVLHQDW |
| SEQ ID NO: 40 | | LNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLT |
| | | VDKSRWQEGNVFSCSVMHEALHNHYTQKSLSLSLG |
| IgG1 wild type | HC | ASTKGPSVFPLAPSSKSTSGGTAALGCLVKDYFPEPVTVSWNSGALTSG |
| SEQ ID NO: 41 | | VHTFPAVLQSSGLYSLSSVVTVPSSSLGTQTYICNVNHKPSNTKVDKRV |
| | | EPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| IgG1 (N297A) | HC | ASTKGPSVFPLAPSSKSTSGGTAALGCLVKDYFPEPVTVSWNSGALTSG |
| mutant constant | | VHTFPAVLQSSGLYSLSSVVTVPSSSLGTQTYICNVNHKPSNTKVDKRV |
| region (EU | | EPKSCDKTHTCPPCPAPELLGGPSVFLFPPKPKDTLMISRTPEVTCVVVD |
| Numbering) | | VSHEDPEVKFNWYVDGVEVHNAKTKPREEQYASTYRVVSVLTVLHQ |
| SEQ ID NO: 42 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| IgM constant | HC | GSASAPTLFPLVSCENSPSDTSSVAVGCLAQDFLPDSITFSWKYKNNSDI |
| delta CDC | | SSTRGFPSVLRGGKYAATSQVLLPSKDVMQGTDEHVVCKVQHPNGNK |
| (P311A, P313S) | | EKNVPLPVIAELPPKVSVFVPPRDGFFGNPRKSKLICQATGFSPRQIQVS |
| SEQ ID NO: 73 | | WLREGKQVGSGVTTDQVQAEAKESGPTTYKVTSTLTIKESDWLGQSM |
| | | FTCRVDHRGLTFQQNASSMCVPDQDTAIRVFAIPPSFASIFLTKSTKLTC |
| | | LVTDLTTYDSVTISWTRQNGEAVKTHTNISESHPNATFSAVGEASICED |
| | | DWNSGERFTCTVTHTDLASSLKQTISRPKGVALHRPDVYLLPPAREQL |
| | | NLRESATITCLVTGFSPADVFVQWMQRGQPLSPEKYVTSAPMPEPQAP |
| | | GRYFAHSILTVSEEEWNTGETYTCVVAHEALPNRVTERTVDKSTGKPT |
| | | LYNVSLVMSDTAGTCY |
| IgGA1 | HC | ASPTSPKVFPLSLCSTQPDGNVVIACLVQGFFPQEPLSVTWSESGQGVT |
| SEQ ID NO: 74 | | ARNFPPSQDASGDLYTTSSQLTLPATQCLAGKSVTCHVKHYTNPSQDV |
| | | TVPCPVPSTPPTPSPSTPPTPSPSCCHPRLSLHRPALEDLLLGSEANLTCT |
| | | LTGLRDASGVTFTWTPSSGKSAVQGPPERDLCGCYSVSSVLPGCAEPW |
| | | NHGKTFTCTAAYPESKTPLTATLSKSGNTFRPEVHLLPPPSEELALNELV |
| | | TLTCLARGFSPKDVLVRWLQGSQELPREKYLTWASRQEPSQGTTTFAV |
| | | TSILRVAAEDWKKGDTFSCMVGHEALPLAFTQKTIDRLAGKPTHVNVS |
| | | VVMAEVDGTCY |
| IgGA2 | HC | ASPTSPKVFPLSLDSTPQDGNVVVACLVQGFFPQEPLSVTWSESGQNVT |
| SEQ ID NO: 75 | | ARNFPPSQDASGDLYTTSSQLTLPATQCPDGKSVTCHVKHYTNSSQDV |
| | | TVPCRVPPPPPCCHPRLSLHRPALEDLLLGSEANLTCTLTGLRDASGATF |
| | | TWTPSSGKSAVQGPPERDLCGCYSVSSVLPGCAQPWNHGETFTCTAAH |
| | | PELKTPLTANITKSGNTFRPEVHLLPPPSEELALNELVTLTCLARGFSPK |
| | | DVLVRWLQGSQELPREKYLTWASRQEPSQGTTTYAVTSILRVAAEDW |
| | | KKGETFSCMVGHEALPLAFTQKTIDRMAGKPTHINVSVVMAEADGTCY |
| Human Ig_J | HC | MKNHLLFWGVLAVFIKAVHVKAQEDERIVLVDNKCKCARITSRIIRSSE |
| chain | | DPNEDIVERNIRIIVPLNNRENISDPTSPLRTRFVYHLSDLCKKCDPTEVE |
| SEQ ID NO: 76 | | LDNQIVTATQSNICDEDSATETCYTYDRNKCYTAVVPLVYGGETKMVE |
| | | TALTPDACYPD |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKKV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYASTYRVVSVLTVLHQ |
| 204 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKKV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYASTYRVVSVLTVLHQ |
| 205 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGKEPEA |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKRV |
| heavy chain | | EPKSCDKTHTCPPCPAPELLGGPSVFLFPPKPKDTLMISRTPEVTCVVVD |
| SEQ ID NO: 41 | | VSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| Human Fc Knob | HC | DKTHTCPPCPAPELLGGPSVFLFPPKPKDTLMISRTPEVTCVVVDVSHE |
| cys N297A | | DPEVKFNWYVDGVEVHNAKTKPREEQYASTYRVVSVLTVLHQDWLN |
| SEQ ID NO: | | GKEYKCKVSNKALPAPIEKTISKAKGQPREPQVYTLPPCREEMTKNQV |
| 206 | | SLWCLVKGFYPSDIAVEWESNGQPENNYKTTPPVLDSDGSFFLYSKLT |
| | | VDKSRWQQGNVFSCSVMHEALHNHYTQKSLSLSPGK |
| Human IgG1 | HC | ASTKGPSVFPLAPSSKSTSGGTAALGCLVKDYFPEPVTVSWNSGALTSG |
| hole cys N297A | | VHTFPAVLQSSGLYSLSSVVTVPSSSLGTQTYICNVNHKPSNTKVDKRV |
| SEQ ID NO: | | EPKSCDKTHTCPPCPAPELLGGPSVFLFPPKPKDTLMISRTPEVTCVVVD |
| 207 | | VSHEDPEVKFNWYVDGVEVHNAKTKPREEQYASTYRVVSVLTVLHQ |
| | | DWLNGKEYKCKVSNKALPAPIEKTISKAKGQPREPQVCTLPPSREEMT |
| | | KNQVSLSCAVKGFYPSDIAVEWESNGQPENNYKTTPPVLDSDGSFFLV |
| | | SKLTVDKSRWQQGNVFSCSVMHEALHNHYTQKSLSLSPGK |

TABLE 4
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

TABLE 5
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

TABLE 6
CTCAE v 4.0 CRS grading scale

| CRS | |
|---|---|
| grade | Characteristics |
| Grade 1 | Mild; No infusion interruption; No intervention |
| Grade 2 | Infusion interruption indicated but responds promptly to |
| | symptomatic treatment (e.g., antihistamines, NSAIDS, narcotics, |
| | IV fluids); prophylactic medications indicated for <=24 hrs |
| Grade 3 | Prolonged (e.g., not rapidly responsive to symptomatic |
| | medications and/or brief interruption of infusion); recurrence |
| | of symptoms following initial improvement; hospitalization |
| | indicated for clinical sequelae (e.g., renal impairment, |
| | pulmonary infiltrates) |
| Grade 4 | Life threatening consequences; pressor or ventilator support |

TABLE 7
NCI CRS grading scale

| CRS | |
|---|---|
| grade | Characteristics |
| Grade 1 | Symptoms are not life threatening and require symptomatic |
| | treatment only; e.g., fever, nausea, fatigue, headache, myalgias, |
| | malaise |
| Grade 2 | Symptoms require and respond to moderate intervention; |
| | Oxygen requirement <40% or hypotension responsive to fluids |
| | or low dose pressors or Grade 2 organ toxicity |
| Grade 3 | Symptoms require and respond to aggressive intervention; |
| | Oxygen requirement >=40% or Hypotension requiring high dose |
| | or multiple pressors or grade 3 organ toxicity or grade 4 |
| | transaminitis |
| Grade 4 | Life threatening symptoms Requirement for ventilator support or |
| | Grade 4; organ toxicity (excluding transaminitis) |

TABLE 8
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

TABLE 9
Exemplary Construct Sequences

| | | | |
|---|---|---|---|
| | SEQ | | |
| Chain | ID NO: | Component | Sequence |

TCRVß/IL15-sushi bispecific

| | | | |
|---|---|---|---|
| Chain 1: | 228 | Full Sequence | QVQLVQSGAEVKKPGSSVKVSCKASGHDFRLTYIHWVRQA |
| TCRVß- | | | PGQGLEWMGRVSAGSGNVKYNEKFKGRVTITADTSTSTAY |
| Fc_Hole_ | | | MELSSLRSEDTAVYYCAVSYYSYDVLDYWGQGTTVTVSSA |
| Cys_N297A | | | STKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| (CDRs | | | VNHKPSNTKVDKRVEPKSCDKTHTCPPCPAPELLGGPSVFL |
| underlined) | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 168 | anti-TCRvß | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 164 | HC CDR1 | GHDFRLTYIH |
| | 229 | HC CDR1 | LTYIH |
| | 234 | HC CDR1 | FRLTYIH |
| | 165 | HC CDR2 | RVSAGSGNVKYNEKFKG |
| | 5 | HC CDR3 | SYYSYDVLDY |
| | 207 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2: | 235 | Full Sequence | ITCPPPMSVEHADIWVKSYSLYSRERYICNSGFKRKAGTSSL |
| IL15RA_ | | | TECVLNKATNVAHWTTPSLKCIRDPALVHQRPAPPSGGSGG |
| Sushi-GS | | | GGSGGGSGGGGSGGNWVNVISDLKKIEDLIQSMHIDATLYT |
| linker-IL15- | | | ESDVHPSCKVTAMKCFLLELQVISLESGDASIHDTVENLIILA |
| GS linker- | | | NNSLSSNGNVTESGCKECEELEEKNIKEFLQSFVHIVQMFIN |
| Fc_Knob_ | | | TSGGGGSGGGGSDKTHTCPPCPAPELLGGPSVFLFPPKPKDT |
| Cys_N297A | | | LMISRTPEVTCVVVDVSHEDPEVKFNWYVDGVEVHNAKTK |
| | | | PREEQYASTYRVVSVLTVLHQDWLNGKEYKCKVSNKALP |
| | | | APIEKTISKAKGQPREPQVYTLPPCREEMTKNQVSLWCLVK |
| | | | GFYPSDIAVEWESNGQPENNYKTTPPVLDSDGSFFLYSKLT |
| | | | VDKSRWQQGNVFSCSVMHEALHNHYTQKSLSLSPGK |
| | 236 | IL15RA Sushi | ITCPPPMSVEHADIWVKSYSLYSRERYICNSGFKRKAGTSSL |
| | | | TECVLNKATNVAHWTTPSLKCIRDPALVHQRPAPP |
| | 243 | linker | SGGSGGGGSGGGSGGGGSGG |
| | 238 | human IL-15 | NWVNVISDLKKIEDLIQSMHIDATLYTESDVHPSCKVTAMK |
| | | | CFLLELQVISLESGDASIHDTVENLIILANNSLSSNGNVTESG |
| | | | CKECEELEEKNIKEFLQSFVHIVQMFINTS |
| | 237 | linker | GGGGSGGGGS |
| | 206 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3: | 239 | Full Sequence | DIQMTQSPSFLSASVGDRVTITCKASQNVADRVVWHQQKP |
| TCRVß light | | | GKAPKALIYSSSHRYKGVPSRFSGSGSGTEFTLTISSLQPEDF |
| chain (CDRs | | | ATYFCQQFKSYPLTFGQGTKLEIKRTVAAPSVFIFPPSDEQL |
| underlined) | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 173 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 171 | LC CDR1 | KASQNVADRVV |
| | 167 | LC CDR2 | SSSHRYK |
| | 8 | LC CDR3 | QQFKSYPLT |
| | 203 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

TCRVß/IL15 bispecific

| | | | |
|---|---|---|---|
| Chain 1: | 228 | Full Sequence | QVQLVQSGAEVKKPGSSVKVSCKASGHDFRLTYIHWVRQA |
| TCRVß- | | | PGQGLEWMGRVSAGSGNVKYNEKFKGRVTITADTSTSTAY |
| Fc_Hole_ | | | MELSSLRSEDTAVYYCAVSYYSYDVLDYWGQGTTVTVSSA |
| Cys_N297A | | | STKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| (CDRs | | | VNHKPSNTKVDKRVEPKSCDKTHTCPPCPAPELLGGPSVFL |
| underlined) | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 168 | anti-TCRvß | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 164 | HC CDR1 | GHDFRLTYIH |
| | 229 | HC CDR1 | LTYIH |
| | 234 | HC CDR1 | FRLTYIH |
| | 165 | HC CDR2 | RVSAGSGNVKYNEKFKG |
| | 5 | HC CDR3 | SYYSYDVLDY |
| | 207 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2: | 240 | Full Sequence | NWVNVISDLKKIEDLIQSMHIDATLYTESDVHPSCKVTAMK |
| IL15-GS | | | CFLLELQVISLESGDASIHDTVENLIILANNSLSSNGNVTESG |
| linker- | | | CKECEELEEKNIKEFLQSFVHIVQMFINTSGGGGSGGGGSDK |
| Fc_Knob_ | | | THTCPPCPAPELLGGPSVFLFPPKPKDTLMISRTPEVTCVVV |
| Cys_N297A | | | DVSHEDPEVKFNWYVDGVEVHNAKTKPREEQYASTYRVV |
| | | | SVLTVLHQDWLNGKEYKCKVSNKALPAPIEKTISKAKGQPR |
| | | | EPQVYTLPPCREEMTKNQVSLWCLVKGFYPSDIAVEWESN |
| | | | GQPENNYKTTPPVLDSDGSFFLYSKLTVDKSRWQQGNVFS |
| | | | CSVMHEALHNHYTQKSLSLSPGK |
| | 238 | human IL-15 | NWVNVISDLKKIEDLIQSMHIDATLYTESDVHPSCKVTAMK |
| | | | CFLLELQVISLESGDASIHDTVENLIILANNSLSSNGNVTESG |
| | | | CKECEELEEKNIKEFLQSFVHIVQMFINTS |
| | 237 | linker | GGGGSGGGGS |
| | 206 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3: | 239 | Full Sequence | DIQMTQSPSFLSASVGDRVTITCKASQNVADRVVWHQQKP |
| TCRVß light | | | GKAPKALIYSSSHRYKGVPSRFSGSGSGTEFTLTISSLQPEDF |
| chain (CDRs | | | ATYFCQQFKSYPLTFGQGTKLEIKRTVAAPSVFIFPPSDEQL |
| underlined) | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 173 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 171 | LC CDR1 | KASQNVADRVV |
| | 167 | LC CDR2 | SSSHRYK |
| | 8 | LC CDR3 | QQFKSYPLT |
| | 203 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

BKM0186

| | | | |
|---|---|---|---|
| Chain 1 | 228 | Full Sequence | QVQLVQSGAEVKKPGSSVKVSCKASGHDFRLTYIHWVRQA |
| (heavy | | | PGQGLEWMGRVSAGSGNVKYNEKFKGRVTITADTSTSTAY |
| chain) | | | MELSSLRSEDTAVYYCAVSYYSYDVLDYWGQGTTVTVSSA |
| (CDRs | | | STKGPSVFPLAPSSKSTSGGTAALGCLVKDYFPEPVTVSWN |
| underlined) | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 168 | anti-TCRvß | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 164 | HC CDR1 | GHDFRLTYIH |
| | 229 | HC CDR1 | LTYIH |
| | 234 | HC CDR1 | FRLTYIH |
| | 165 | HC CDR2 | RVSAGSGNVKYNEKFKG |
| | 5 | HC CDR3 | SYYSYDVLDY |
| | 207 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2 | 241 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSPG |
| | | | K |
| | 242 | IL-2 C125A | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLT |
| | 237 | linker | GGGGSGGGGS |
| | 206 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3 | 239 | Full Sequence | DIQMTQSPSFLSASVGDRVTITCKASQNVADRVVWHQQKP |
| (light chain) | | | GKAPKALIYSSSHRYKGVPSRFSGSGSGTEFTLTISSLQPEDF |
| (CDRs | | | ATYFCQQFKSYPLTFGQGTKLEIKRTVAAPSVFIFPPSDEQL |
| underlined) | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 173 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 171 | LC CDR1 | KASQNVADRVV |
| | 167 | LC CDR2 | SSSHRYK |
| | 8 | LC CDR3 | QQFKSYPLT |
| | 203 | Immunoglobulin | \|RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

mBKM0186

| | | | |
|---|---|---|---|
| Chain 1 | 244 | Full Sequence | QVQLQQSGTELMKPGASVKISCKASGYTFSNYWIEWIKQRP |
| (heavy | | | GHGLEWVGEILPGAGPTNYNEKFKGKATFTADSSSNTAYM |
| chain) | | | QLSSLTSEDSAVYYCARTDYDYDWFAYWGQGTLVTVSAA |
| (CDRs | | | KTTAPSVYPLAPVSGDTTGSSVTLGCLVKGYFPEPVTLTWN |
| underlined) | | | SGSLSSGVHTFPAVLQSDLYTLSSSVTVTSSTWPSQSITCNV |
| | | | AHPASSTKVDKKIEPKSCDKTHTCPPCPAPNAAGGPSVFIFP |
| | | | PKIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVCVLPPPEEEMTKKQVTLSCA |
| | | | VTDFMPEDIYVEWTNNGKTELNYKNTEPVLDSDGSYFMVS |
| | | | KLRVEKKNWVERNSYSCSVVHEGLHNHHTTKSFSRTPGKE |
| | | | PEA |
| | 245 | Full Sequence | QVQLQQSGTELMKPGASVKISCKASGYTFSNYWIEWIKQRP |
| | | without tag | GHGLEWVGEILPGAGPTNYNEKFKGKATFTADSSSNTAYM |
| | | | QLSSLTSEDSAVYYCARTDYDYDWFAYWGQGTLVTVSAA |
| | | | KTTAPSVYPLAPVSGDTTGSSVTLGCLVKGYFPEPVTLTWN |
| | | | SGSLSSGVHTFPAVLQSDLYTLSSSVTVTSSTWPSQSITCNV |
| | | | AHPASSTKVDKKIEPKSCDKTHTCPPCPAPNAAGGPSVFIFP |
| | | | PKIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVCVLPPPEEEMTKKQVTLSCA |
| | | | VTDFMPEDIYVEWTNNGKTELNYKNTEPVLDSDGSYFMVS |
| | | | KLRVEKKNWVERNSYSCSVVHEGLHNHHTTKSFSRTPGK |
| | 246 | anti-TCRvß | QVQLQQSGTELMKPGASVKISCKASGYTFSNYWIEWIKQRP |
| | | VH | GHGLEWVGEILPGAGPTNYNEKFKGKATFTADSSSNTAYM |
| | | from murine | QLSSLTSEDSAVYYCARTDYDYDWFAYWGQGTLVTVSAA |
| | | MR5-2 | KTTAPSVYPLAPVSGDTTGSSVTLGCLVKGYFPEPVTLTWN |
| | | | SGSLSSGVHTFPAVLQSDLYTLSSSVTVTSS |
| | 247 | HC CDR1 | GYTFSNYWIE |
| | 248 | HC CDR2 | EILPGAGPTNYNEKFKG |
| | 249 | HC CDR3 | TDYDYDWFAY |
| | 264 | mouse Fc | LDSDGSYFMVSKLRVEKKNWVERNSYSCSVVHEGLHNHH |
| | | heavy chain | TWPSQSITCNVAHPASSTKVDKKIEPKSCDKTHTCPPCPAPN |
| | | | AAGGPSVFIFPPKIKDVLMISLSPIVTCVVVDVSEDDPDVQIS |
| | | comprising | WFVNNVEVHTAQTQTHREDYNSTLRVVSALPIQHQDWMS |
| | | LALAPG | GKEFKCKVNNKDLGAPIERTISKPKGSVRAPQVCVLPPPEEE |
| | | mutations | MTKKQVTLSCAVTDFMPEDIYVEWTNNGKTELNYKNTEPV |
| | | (L234A L235A | TTKSFSRTPGK |
| | | and P329G) | |
| | 265 | Tag | EPEA |
| Chain 2 | 266 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLTGGGGSGGGGSDKTHTCPPCPAPNAAGGPSVFIFPP |
| | | | KIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVYVLPPCEEEMTKKQVTLWC |
| | | | MVTDFMPEDIYVEWTNNGKTELNYKNTEPVLDSDGSYFMY |
| | | | SKLRVEKKNWVERNSYSCSVVHEGLHNHHTTKSFSRTPGK |
| | | | GGGGSHHHHHHHH |
| | 267 | Full Sequence | APTSSSTKKTQLQLEHLLLDLQMILNGINNYKNPKLTRMLT |
| | | without tag | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLTGGGGSGGGGSDKTHTCPPCPAPNAAGGPSVFIFPP |
| | | | KIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVYVLPPCEEEMTKKQVTLWC |
| | | | MVTDFMPEDIYVEWTNNGKTELNYKNTEPVLDSDGSYFMY |
| | | | SKLRVEKKNWVERNSYSCSVVHEGLHNHHTTKSFSRTPGK |
| | 268 | IL-2 | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLT |
| | 237 | linker | GGGGSGGGGS |
| | 269 | mouse Fc | DKTHTCPPCPAPNAAGGPSVFIFPPKIKDVLMISLSPIVTCVV |
| | | heavy chain | VDVSEDDPDVQISWFVNNVEVHTAQTQTHREDYNSTLRVV |
| | | comprising | SALPIQHQDWMSGKEFKCKVNNKDLGAPIERTISKPKGSVR |
| | | LALAPG | APQVYVLPPCEEEMTKKQVTLWCMVTDFMPEDIYVEWTN |
| | | mutations | NGKTELNYKNTEPVLDSDGSYFMYSKLRVEKKNWVERNS |
| | | (L234A L235A | YSCSVVHEGLHNHHTTKSFSRTPGK |
| | | and P329G) | |
| | 270 | Tag | GGGGSHHHHHHHH |
| Chain 3 | 271 | Full Sequence | DIVMSQSPSSLAVSVGEKVTMSCKSSQSLLYSGNQKNYLA |
| (light chain) | | | WYQQKPGQSPKLLIYWASTRESGVPDRFTGSGSGTDFTLTI |
| (CDRs | | | NSVKAEDLTVYYCQQYYGYPRTFGGGTKVEIKRADAAPTV |
| underlined) | | | SIFPPSSEQLTSGGASVVCFLNNFYPKDINVKWKIDGSERQN |
| | | | GVLNSWTDQDSKDSTYSMSSTLTLTKDEYERHNSYTCEAT |
| | | | HKTSTSPIVKSFNRNEC |
| | 252 | anti-TCRvß | DIVMSQSPSSLAVSVGEKVTMSCKSSQSLLYSGNQKNYLA |
| | | VL | WYQQKPGQSPKLLIYWASTRESGVPDRFTGSGSGTDFTLTI |
| | | from murine | NSVKAEDLTVYYCQQYYGYPRTFGGGTKVEIK |
| | | MR5-2 | |
| | 253 | LC CDR1 | KSSQSLLYSGN |
| | 254 | LC CDR2 | PKLLIYWA |
| | 255 | LC CDR3 | QQYYGYPRT |
| | 256 | Immunoglobulin | RADAAPTVSIFPPSSEQLTSGGASVVCFLNNFYPKDINVKWK |
| | | constant | IDGSERQNGVLNSWTDQDSKDSTYSMSSTLTLTKDEYERHN |
| | | region | SYTCEATHKTSTSPIVKSFNRNEC |

mBKM0281

| | | | |
|---|---|---|---|
| Chain 1 | 257 | Full Sequence | QVQLQQSGTELMKPGASVKISCKASGYTFSNYWIEWIKQRP |
| (heavy | | | GHGLEWVGEILPGAGPTNYNEKFKGKATFTADSSSNTAYM |
| chain) | | | QLSSLTSEDSAVYYCARTDYDYDWFAYWGQGTLVTVSAA |
| (CDRs | | | KTTAPSVYPLAPVSGDTTGSSVTLGCLVKGYFPEPVTLTWN |
| underlined) | | | SGSLSSGVHTFPAVLQSDLYTLSSSVTVTSSTWPSQSITCNV |
| | | | AHPASSTKVDKKIEPKSCDKTHTCPPCPAPNAAGGPSVFIFP |
| | | | PKIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVYVLPPPEEEMTKKQVTLTC |
| | | | MVTDFMPEDIYVEWTNNGKTELNYKNTEPVLDSDGSYFMY |
| | | | SKLRVEKKNWVERNSYSCSVVHEGLHNHHTTKSFSRTPGK |
| | | | EPEA |
| | 246 | anti-TCRvß | QVQLQQSGTELMKPGASVKISCKASGYTFSNYWIEWIKQRP |
| | | VH | GHGLEWVGEILPGAGPTNYNEKFKGKATFTADSSSNTAYM |
| | | from murine | QLSSLTSEDSAVYYCARTDYDYDWFAYWGQGTLVTVSAA |
| | | MR5-2 | KTTAPSVYPLAPVSGDTTGSSVTLGCLVKGYFPEPVTLTWN |
| | | | SGSLSSGVHTFPAVLQSDLYTLSSSVTVTSS |
| | 247 | HC CDR1 | GYTFSNYWIE |
| | 248 | HC CDR2 | EILPGAGPTNYNEKFKG |
| | 249 | HC CDR3 | TDYDYDWFAY |
| | 250 | mouse Fc | TWPSQSITCNVAHPASSTKVDKKIEPKSCDKTHTCPPCPAPN |
| | | heavy chain | AAGGPSVFIFPPKIKDVLMISLSPIVTCVVVDVSEDDPDVQIS |
| | | comprising | WFVNNVEVHTAQTQTHREDYNSTLRVVSALPIQHQDWMS |
| | | LALAPG | GKEFKCKVNNKDLGAPIERTISKPKGSVRAPQVYVLPPPEEE |
| | | mutations | MTKKQVTLTCMVTDFMPEDIYVEWTNNGKTELNYKNTEP |
| | | (L234A L235A | VLDSDGSYFMYSKLRVEKKNWVERNSYSCSVVHEGLHNH |
| | | and P329G) | HTTKSFSRTPGKEPEA |
| Chain 2 | 251 | Full Sequence | DIVMSQSPSSLAVSVGEKVTMSCKSSQSLLYSGNQKNYLA |
| (light chain- | | | WYQQKPGQSPKLLIYWASTRESGVPDRFTGSGSGTDFTLTI |
| IL2) | | | NSVKAEDLTVYYCQQYYGYPRTFGGGTKVEIKRADAAPTV |
| (CDRs | | | SIFPPSSEQLTSGGASVVCFLNNFYPKDINVKWKIDGSERQN |
| underlined) | | | GVLNSWTDQDSKDSTYSMSSTLTLTKDEYERHNSYTCEAT |
| | | | HKTSTSPIVKSFNRNECGGGGSGGGGSGGGGSAPTSSSTKK |
| | | | TQLQLEHLLLDLQMILNGINNYKNPKLTRMLTFKFYMPKK |
| | | | ATELKHLQCLEEELKPLEEVLNLAQSKNFHLRPRDLISNINVI |
| | | | VLELKGSETTFMCEYADETATIVEFLNRWITFCQSIISTLT |
| | 252 | anti-TCRvß | DIVMSQSPSSLAVSVGEKVTMSCKSSQSLLYSGNQKNYLA |
| | | VL | WYQQKPGQSPKLLIYWASTRESGVPDRFTGSGSGTDFTLTI |
| | | from murine | NSVKAEDLTVYYCQQYYGYPRTFGGGTKVEIK |
| | | MR5-2 | |
| | 253 | LC CDR1 | KSSQSLLYSGN |
| | 254 | LC CDR2 | PKLLIYWA |
| | 255 | LC CDR3 | QQYYGYPRT |
| | 256 | Immunoglobulin | \|RADAAPTVSIFPPSSEQLTSGGASVVCFLNNFYPKDINVKWK |
| | | constant | IDGSERQNGVLNSWTDQDSKDSTYSMSSTLTLTKDEYERHN |
| | | region | SYTCEATHKTSTSPIVKSFNRNEC |
| | 258 | linker | GGGGSGGGGSGGGGS |
| | 268 | IL-2 | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLT |

BMM0317 (IL-7)

| | | | |
|---|---|---|---|
| Chain 1: | 228 | Full Sequence | QVQLVQSGAEVKKPGSSVKVSCKASGHDFRLTYIHWVRQA |
| TCRVß- | | | PGQGLEWMGRVSAGSGNVKYNEKFKGRVTITADTSTSTAY |
| Fc_Hole_ | | | MELSSLRSEDTAVYYCAVSYYSYDVLDYWGQGTTVTVSSA |
| Cys_N297A | | | STKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| (CDRs | | | VNHKPSNTKVDKRVEPKSCDKTHTCPPCPAPELLGGPSVFL |
| underlined) | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 168 | anti-TCRvß | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 164 | HC CDR1 | GHDFRLTYIH |
| | 229 | HC CDR1 | LTYIH |
| | 234 | HC CDR1 | FRLTYIH |
| | 165 | HC CDR2 | RVSAGSGNVKYNEKFKG |
| | 5 | HC CDR3 | SYYSYDVLDY |
| | 207 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2: | 272 | Full Sequence | DCDIEGKDGKQYESVLMVSIDQLLDSMKEIGSNCLNNEFNF |
| IL-7-GS | | | FKRHICDANKEGMFLFRAARKLRQFLKMNSTGDFDLHLLK |
| linker-IL15 | | | VSEGTTILLNCTGQVKGRKPAALGEAQPTKSLEENKSLKEQ |
| -GS linker- | | | KKLNDLCFLKRLLQEIKTCWNKILMGTKEHGGGGSGGGGS |
| Fc_Knob_ | | | DKTHTCPPCPAPELLGGPSVFLFPPKPKDTLMISRTPEVTCV |
| Cys_N297A | | | VVDVSHEDPEVKFNWYVDGVEVHNAKTKPREEQYASTYR |
| | | | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| | 273 | IL-7 | DCDIEGKDGKQYESVLMVSIDQLLDSMKEIGSNCLNNEFNF |
| | | | FKRHICDANKEGMFLFRAARKLRQFLKMNSTGDFDLHLLK |
| | | | VSEGTTILLNCTGQVKGRKPAALGEAQPTKSLEENKSLKEQ |
| | | | KKLNDLCFLKRLLQEIKTCWNKILMGTKEH |
| | 237 | linker | GGGGSGGGGS |
| | 20 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3: | 239 | Full Sequence | DIQMTQSPSFLSASVGDRVTITCKASQNVADRVVWHQQKP |
| TCRVß light | | | GKAPKALIYSSSHRYKGVPSRFSGSGSGTEFTLTISSLQPEDF |
| chain (CDRs | | | ATYFCQQFKSYPLTFGQGTKLEIKRTVAAPSVFIFPPSDEQL |
| underlined) | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 173 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 171 | LC CDR1 | KASQNVADRVV |
| | 167 | LC CDR2 | SSSHRYK |
| | 8 | LC CDR3 | QQFKSYPLT |
| | 203 | Immunoglobuli | RTVAAPSVFIFPPSDEQLKSGTASVVCLLNNFYPREAKVQW |
| | | In kappa | KVDNALQSGNSQESVTEQDSKDSTYSLSSTLTLSKADYEKH |
| | | constant C | KVYACEVTHQGLSSPVTKSFNRGEC |
| | | region | |

BLM0319 (IL-12)

| | | | |
|---|---|---|---|
| Chain 1: | 228 | Full Sequence | QVQLVQSGAEVKKPGSSVKVSCKASGHDFRLTYIHWVRQA |
| TCRVß- | | | PGQGLEWMGRVSAGSGNVKYNEKFKGRVTITADTSTSTAY |
| Fc_Hole_ | | | MELSSLRSEDTAVYYCAVSYYSYDVLDYWGQGTTVTVSSA |
| Cys_N297A | | | STKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| (CDRs | | | VNHKPSNTKVDKRVEPKSCDKTHTCPPCPAPELLGGPSVFL |
| underlined) | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 168 | anti-TCRvß | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 164 | HC CDR1 | GHDFRLTYIH |
| | 229 | HC CDR1 | LTYIH |
| | 234 | HC CDR1 | FRLTYIH |
| | 165 | HC CDR2 | RVSAGSGNVKYNEKFKG |
| | 5 | HC CDR3 | SYYSYDVLDY |
| | 207 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2: | 275 | Full Sequence | IWELKKDVYVVELDWYPDAPGEMVVLTCDTPEEDGITWTL |
| IL-12-GS | | | DQSSEVLGSGKTLTIQVKEFGDAGQYTCHKGGEVLSHSLLL |
| linker-IL15- | | | LHKKEDGIWSTDILKDQKEPKNKTFLRCEAKNYSGRFTCW |
| GS linker- | | | WLTTISTDLTFSVKSSRGSSDPQGVTCGAATLSAERVRGDN |
| Fc_Knob_ | | | KEYEYSVECQEDSACPAAEESLPIEVMVDAVHKLKYENYTS |
| Cys_N297A | | | SFFIRDIIKPDPPKNLQLKPLKNSRQVEVSWEYPDTWSTPHS |
| | | | YFSLTFCVQVQGKSKREKKDRVFTDKTSATVICRKNASISV |
| | | | RAQDRYYSSSWSEWASVPCSGGGGSGGGGSGGGGSRNLPV |
| | | | ATPDPGMFPCLHHSQNLLRAVSNMLQKARQTLEFYPCTSEE |
| | | | IDHEDITKDKTSTVEACLPLELTKNESCLNSRETSFITNGSCL |
| | | | ASRKTSFMMALCLSSIYEDLKMYQVEFKTMNAKLLMDPKR |
| | | | QIFLDQNMLAVIDELMQALNFNSETVPQKSSLEEPDFYKTKI |
| | | | KLCILLHAFRIRAVTIDRVMSYLNASGGGGSGGGGSDKTHT |
| | | | CPPCPAPELLGGPSVFLFPPKPKDTLMISRTPEVTCVVVDVS |
| | | | HEDPEVKFNWYVDGVEVHNAKTKPREEQYASTYRVVSVL |
| | | | TVLHQDWLNGKEYKCKVSNKALPAPIEKTISKAKGQPREPQ |
| | | | VYTLPPCREEMTKNQVSLWCLVKGFYPSDIAVEWESNGQP |
| | | | ENNYKTTPPVLDSDGSFFLYSKLTVDKSRWQQGNVFSCSV |
| | | | MHEALHNHYTQKSLSLSPGK |
| | 276 | IL-12 | IWELKKDVYVVELDWYPDAPGEMVVLTCDTPEEDGITWTL |
| | | | DQSSEVLGSGKTLTIQVKEFGDAGQYTCHKGGEVLSHSLLL |
| | | | LHKKEDGIWSTDILKDQKEPKNKTFLRCEAKNYSGRFTCW |
| | | | WLTTISTDLTFSVKSSRGSSDPQGVTCGAATLSAERVRGDN |
| | | | KEYEYSVECQEDSACPAAEESLPIEVMVDAVHKLKYENYTS |
| | | | SFFIRDIIKPDPPKNLQLKPLKNSRQVEVSWEYPDTWSTPHS |
| | | | YFSLTFCVQVQGKSKREKKDRVFTDKTSATVICRKNASISV |
| | | | RAQDRYYSSSWSEWASVPCSGGGGSGGGGSGGGGSRNLPV |
| | | | ATPDPGMFPCLHHSQNLLRAVSNMLQKARQTLEFYPCTSEE |
| | | | IDHEDITKDKTSTVEACLPLELTKNESCLNSRETSFITNGSCL |
| | | | ASRKTSFMMALCLSSIYEDLKMYQVEFKTMNAKLLMDPKR |
| | | | QIFLDQNMLAVIDELMQALNFNSETVPQKSSLEEPDFYKTKI |
| | | | KLCILLHAFRIRAVTIDRVMSYLNAS |
| | 277 | IL-12 beta | IWELKKDVYVVELDWYPDAPGEMVVLTCDTPEEDGITWTL |
| | | subunit | DQSSEVLGSGKTLTIQVKEFGDAGQYTCHKGGEVLSHSLLL |
| | | | LHKKEDGIWSTDILKDQKEPKNKTFLRCEAKNYSGRFTCW |
| | | | WLTTISTDLTFSVKSSRGSSDPQGVTCGAATLSAERVRGDN |
| | | | KEYEYSVECQEDSACPAAEESLPIEVMVDAVHKLKYENYTS |
| | | | SFFIRDIIKPDPPKNLQLKPLKNSRQVEVSWEYPDTWSTPHS |
| | | | YFSLTFCVQVQGKSKREKKDRVFTDKTSATVICRKNASISV |
| | | | RAQDRYYSSSWSEWASVPC |
| | 278 | Linker | SGGGGSGGGGSGGGGS |
| | 279 | IL-12 alpha | RNLPVATPDPGMFPCLHHSQNLLRAVSNMLQKARQTLEFY |
| | | subunit | PCTSEEIDHEDITKDKTSTVEACLPLELTKNESCLNSRETSFIT |
| | | | NGSCLASRKTSFMMALCLSSIYEDLKMYQVEFKTMNAKLL |
| | | | MDPKRQIFLDQNMLAVIDELMQALNFNSETVPQKSSLEEPD |
| | | | FYKTKIKLCILLHAFRIRAVTIDRVMSYLNAS |
| | 237 | linker | GGGGSGGGGS |
| | 206 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | IN297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3: | 239 | Full Sequence | DIQMTQSPSFLSASVGDRVTITCKASQNVADRVVWHQQKP |
| | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| TCRVß light | 173 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| chain (CDRs | 171 | LC CDR1 | KASQNVADRVV |
| underlined) | 167 | LC CDR2 | SSSHRYK |
| | 8 | LC CDR3 | QQFKSYPLT |
| | 203 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

BLM0321 (IL-21)

| | | | |
|---|---|---|---|
| Chain 1: | 228 | Full Sequence | QVQLVQSGAEVKKPGSSVKVSCKASGHDFRLTYIHWVRQA |
| TCRVß- | | | PGQGLEWMGRVSAGSGNVKYNEKFKGRVTITADTSTSTAY |
| Fc_Hole_ | | | MELSSLRSEDTAVYYCAVSYYSYDVLDYWGQGTTVTVSSA |
| Cys_N297A | | | STKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| (CDRs | | | VNHKPSNTKVDKRVEPKSCDKTHTCPPCPAPELLGGPSVFL |
| underlined) | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 168 | anti-TCRvß | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 164 | HC CDR1 | GHDFRLTYIH |
| | 229 | HC CDR1 | LTYIH |
| | 234 | HC CDR1 | FRLTYIH |
| | 165 | HC CDR2 | RVSAGSGNVKYNEKFKG |
| | 5 | HC CDR3 | SYYSYDVLDY |
| | 207 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2: | 288 | Full Sequence | QGQDRHMIRMRQLIDIVDQLKNYVNDLVPEFLPAPEDVETN |
| IL-21-GS | | | CEWSAFSCFQKAQLKSANTGNNERIINVSIKKLKRKPPSTNA |
| linker-IL15- | | | GRRQKHRLTCPSCDSYEKKPPKEFLERFKSLLQKMIHQHLS |
| GS linker- | | | SRTHGSEDSGGGGSGGGGSDKTHTCPPCPAPELLGGPSVFL |
| Fc_Knob_ | | | FPPKPKDTLMISRTPEVTCVVVDVSHEDPEVKFNWYVDGV |
| Cys_N297A | | | EVHNAKTKPREEQYASTYRVVSVLTVLHQDWLNGKEYKC |
| | | | KVSNKALPAPIEKTISKAKGQPREPQVYTLPPCREEMTKNQ |
| | | | VSLWCLVKGFYPSDIAVEWESNGQPENNYKTTPPVLDSDGS |
| | | | FFLYSKLTVDKSRWQQGNVFSCSVMHEALHNHYTQKSLSL |
| | | | SPGK |
| | 274 | IL-21 | QGQDRHMIRMRQLIDIVDQLKNYVNDLVPEFLPAPEDVETN |
| | | | CEWSAFSCFQKAQLKSANTGNNERIINVSIKKLKRKPPSTNA |
| | | | GRRQKHRLTCPSCDSYEKKPPKEFLERFKSLLQKMIHQHLS |
| | | | SRTHGSEDS |
| | 237 | linker | GGGGSGGGGS |
| | 206 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3: | 239 | Full Sequence | DIQMTQSPSFLSASVGDRVTITCKASQNVADRVVWHQQKP |
| TCRVß light | | | GKAPKALIYSSSHRYKGVPSRFSGSGSGTEFTLTISSLQPEDF |
| chain (CDRs | | | ATYFCQQFKSYPLTFGQGTKLEIKRTVAAPSVFIFPPSDEQL |
| underlined) | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 173 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 171 | LC CDR1 | KASQNVADRVV |
| | 167 | LC CDR2 | SSSHRYK |
| | 8 | LC CDR3 | QQFKSYPLT |
| | 203 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

TABLE 10
Exemplary multispecific molecule that comprise a first domain that binds to a first
target molecule, wherein the exemplary target molecule comprises TCRVβ 6 (e.g., TCRVβ 6-5,
TCRVβ 6-2). Exemplary TCRβV comprises TCRVβ 6 (e.g., TCRVβ 6-5). Exemplary anti-
TCRVβ 6 antibodies include, but are not limited to, BMM0306, BMM0354, BMM0356,
BMM0307, BMM0359, BMM0355, BMM0308, BMM0360, and BMM0357 which bind to
human TCRVβ 6 (e.g., TCRVβ 6-5). CDRs are underlined.
BMM0306

| | | |
|---|---|---|
| SEQ ID NO: | Heavy chain (VH- | QVQLVQSGAEVKKPGSSVKVSCKASGHDFRLTYIHWV |
| 289 | CH)-Hole | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYASTYRV |
| | | VSVLTVLHQDWLNGKEYKCKVSNKALPAPIEKTISKAK |
| | | GQPREPQVCTLPPSREEMTKNQVSLSCAVKGFYPSDIAV |
| | | EWESNGQPENNYKTTPPVLDSDGSFFLVSKLTVDKSRW |
| | | QQGNVFSCSVMHEALHNHYTQKSLSLSP |
| SEQ ID NO: | Light chain (VL- | DIQMTQSPSFLSASVGDRVTITCKASQNVADRVVWHQQ |
| 239 | CL) | KPGKAPKALIYSSSHRYKGVPSRFSGSGSGTEFTLTISSL |
| | | QPEDFATYFCQQFKSYPLTFGQGTKLEIKRTVAAPSVFIF |
| | | PPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| SEQ ID NO: | IL-15Rαsushi-IL-15 | ITCPPPMSVEHADIWVKSYSLYSRERYICNSGFKRKAGT |
| 292 | Heavy Chain | SSLTECVLNKATNVAHWTTPSLKCIRDPALVHQRPAPPS |
| | Constant Domain | GGSGGGGSGGGSGGGGSGGGNWVNVISDLKKIEDLIQS |
| | (Fc Region)- | MHIDATLYTESDVHPSCKVTAMKCFLLELQVISLESGD |
| | Knob | ASIHDTVENLIILANNSLSSNGNVTESGCKECEELEEKNI |
| | | KEFLQSFVHIVQMFINTSGGGGSGGGGSDKTHTCPPCPA |
| | | PELLGGPSVFLFPPKPKDTLMISRTPEVTCVVVDVSHED |
| | | PEVKFNWYVDGVEVHNAKTKPREEQYASTYRVVSVLT |
| | | VLHQDWLNGKEYKCKVSNKALPAPIEKTISKAKGQPRE |
| | | PQVYTLPPCREEMTKNQVSLWCLVKGFYPSDIAVEWES |
| | | NGQPENNYKTTPPVLDSDGSFFLYSKLTVDKSRWQQG |
| | | NVFSCSVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | SSSHRYK |
| 167 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 173 | | KPGKAPKALIYSSSHRYKGVPSRFSGSGSGTEFTLTISSLQ |
| | | PEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 238 | | MKCFLLELQVISLESGDASIHDTVENLIILANNSLSSNGN |
| | | VTESGCKECEELEEKNIKEFLQSFVHIVQMFINTS |
| SEQ ID NO: | IL-15Rαsushi-IL-15 | ITCPPPMSVEHADIWVKSYSLYSRERYICNSGFKRKAGTS |
| 297 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVENLIILANNSLSSNGNVTESGCKECEELEEKNIKEF |
| | | LQSFVHIVQMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0354

| | | |
|---|---|---|
| SEQ ID NO: | Heavy chain (VH- | QVQLVQSGAEVKKPGSSVKVSCKASGHDFRLTYIHWV |
| 289 | CH)-Hole | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYASTYRV |
| | | VSVLTVLHQDWLNGKEYKCKVSNKALPAPIEKTISKAK |
| | | GQPREPQVCTLPPSREEMTKNQVSLSCAVKGFYPSDIAV |
| | | EWESNGQPENNYKTTPPVLDSDGSFFLVSKLTVDKSRW |
| | | QQGNVFSCSVMHEALHNHYTQKSLSLSP |
| SEQ ID NO: | Light chain (VL- | DIQMTQSPSFLSASVGDRVTITCKASQNVADRVVWHQQ |
| 298 | CL) | KPGKAPKALIYAASYLQSGVPSRFSGSGSGTEFTLTISSL |
| | | QPEDFATYFCQQFKSYPLTFGQGTKLEIKRTVAAPSVFIF |
| | | PPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| SEQ ID NO: | IL-15Rαsushi-IL-15 | ITCPPPMSVEHADIWVKSYSLYSRERYICNSGFKRKAGT |
| 292 | Heavy Chain | SSLTECVLNKATNVAHWTTPSLKCIRDPALVHQRPAPPS |
| | Constant Domain | GGSGGGGSGGGSGGGGSGGGNWVNVISDLKKIEDLIQS |
| | (Fc Region)- | MHIDATLYTESDVHPSCKVTAMKCFLLELQVISLESGD |
| | Knob | ASIHDTVENLIILANNSLSSNGNVTESGCKECEELEEKNI |
| | | KEFLQSFVHIVQMFINTSGGGGSGGGGSDKTHTCPPCPA |
| | | PELLGGPSVFLFPPKPKDTLMISRTPEVTCVVVDVSHED |
| | | PEVKFNWYVDGVEVHNAKTKPREEQYASTYRVVSVLT |
| | | VLHQDWLNGKEYKCKVSNKALPAPIEKTISKAKGQPRE |
| | | PQVYTLPPCREEMTKNQVSLWCLVKGFYPSDIAVEWES |
| | | NGQPENNYKTTPPVLDSDGSFFLYSKLTVDKSRWQQG |
| | | NVFSCSVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | AASYLQS |
| 299 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 300 | | KPGKAPKALIYAASYLQSGVPSRFSGSGSGTEFTLTISSL |
| | | QPEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 238 | | MKCFLLELQVISLESGDASIHDTVENLIILANNSLSSNGN |
| | | VTESGCKECEELEEKNIKEFLQSFVHIVQMFINTS |
| SEQ ID NO: | IL-15Rαsushi-IL-15 | ITCPPPMSVEHADIWVKSYSLYSRERYICNSGFKRKAGTS |
| 297 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVENLIILANNSLSSNGNVTESGCKECEELEEKNIKEF |
| | | LQSFVHIVQMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0356

| | | |
|---|---|---|
| SEQ ID NO: | Heavy chain (VH- | QVQLVQSGAEVKKPGSSVKVSCKASGHDFRLTYIHWV |
| 289 | CH)-Hole | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYASTYRV |
| | | VSVLTVLHQDWLNGKEYKCKVSNKALPAPIEKTISKAK |
| | | GQPREPQVCTLPPSREEMTKNQVSLSCAVKGFYPSDIAV |
| | | EWESNGQPENNYKTTPPVLDSDGSFFLVSKLTVDKSRW |
| | | QQGNVFSCSVMHEALHNHYTQKSLSLSP |
| SEQ ID NO: | Light chain (VL- | DIQMTQSPSFLSASVGDRVTITCKASQNVADRVVWHQQ |
| 301 | CL) | KPGKAPKALIFAASYLQSGVPSRFSGSGSGTEFTLTISSL |
| | | QPEDFATYFCQQFKSYPLTFGQGTKLEIKRTVAAPSVFIF |
| | | PPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| SEQ ID NO: | IL-15Rαsushi-IL-15 | ITCPPPMSVEHADIWVKSYSLYSRERYICNSGFKRKAGT |
| 292 | Heavy Chain | SSLTECVLNKATNVAHWTTPSLKCIRDPALVHQRPAPPS |
| | Constant Domain | GGSGGGGSGGGSGGGGSGGGNWVNVISDLKKIEDLIQS |
| | (Fc Region)- | MHIDATLYTESDVHPSCKVTAMKCFLLELQVISLESGD |
| | Knob | ASIHDTVENLIILANNSLSSNGNVTESGCKECEELEEKNI |
| | | KEFLQSFVHIVQMFINTSGGGGSGGGGSDKTHTCPPCPA |
| | | PELLGGPSVFLFPPKPKDTLMISRTPEVTCVVVDVSHED |
| | | PEVKFNWYVDGVEVHNAKTKPREEQYASTYRVVSVLT |
| | | VLHQDWLNGKEYKCKVSNKALPAPIEKTISKAKGQPRE |
| | | PQVYTLPPCREEMTKNQVSLWCLVKGFYPSDIAVEWES |
| | | NGQPENNYKTTPPVLDSDGSFFLYSKLTVDKSRWQQG |
| | | NVFSCSVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | AASYLQS |
| 299 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 303 | | KPGKAPKALIFAASYLQSGVPSRFSGSGSGTEFTLTISSLQ |
| | | PEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 238 | | MKCFLLELQVISLESGDASIHDTVENLIILANNSLSSNGN |
| | | VTESGCKECEELEEKNIKEFLQSFVHIVQMFINTS |
| SEQ ID NO: | IL-15Rαsushi-IL-15 | ITCPPPMSVEHADIWVKSYSLYSRERYICNSGFKRKAGTS |
| 297 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVENLIILANNSLSSNGNVTESGCKECEELEEKNIKEF |
| | | LQSFVHIVQMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0307

| | | |
|---|---|---|
| SEQ ID NO: | Heavy chain (VH- | QVQLVQSGAEVKKPGSSVKVSCKASGHDFRLTYIHWVR |
| 289 | CH)-Hole | QAPGQGLEWMGRVSAGSGNVKYNEKFKGRVTITADTS |
| | | TSTAYMELSSLRSEDTAVYYCAVSYYSYDVLDYWGQG |
| | | TTVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKRVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSV |
| | | LTVLHQDWLNGKEYKCKVSNKALPAPIEKTISKAKGQP |
| | | REPQVCTLPPSREEMTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSP |
| SEQ ID NO: | Light chain (VL- | DIQMTQSPSFLSASVGDRVTITCKASQNVADRVVWHQQ |
| 239 | CL) | KPGKAPKALIYSSSHRYKGVPSRFSGSGSGTEFTLTISSLQ |
| | | PEDFATYFCQQFKSYPLTFGQGTKLEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| SEQ ID NO: | IL-15Rαsushi-IL-15 | ITCPPPMSVEHADIWVKSYSLYSRERYICNSGFKRKAGTS |
| 304 | Heavy Chain | SLTECVLNKATNVAHWTTPSLKCIRDPALVHQRPAPPSG |
| | Constant Domain | GSGGGGSGGGSGGGGSGGGNWVNVISDLKKIEDLIQSM |
| | (Fc Region)- | HIDATLYTESDVHPSCKVTAMKCFLLELQVISLESGDASI |
| | Knob | HDTVENLIILANNSLSSNGNVTESGCKECEELEEKNIKEF |
| | | LQSFVHIVAMFINTSGGGGSGGGGSDKTHTCPPCPAPEL |
| | | LGGPSVFLFPPKPKDTLMISRTPEVTCVVVDVSHEDPEV |
| | | KFNWYVDGVEVHNAKTKPREEQYASTYRVVSVLTVLH |
| | | QDWLNGKEYKCKVSNKALPAPIEKTISKAKGQPREPQV |
| | | YTLPPCREEMTKNQVSLWCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | SSSHRYK |
| 167 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 173 | | KPGKAPKALIYSSSHRYKGVPSRFSGSGSGTEFTLTISSLQ |
| | | PEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hing- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 307 | | MKCFLLELQVISLESGDASIHDTVENLIILANNSLSSNGN |
| | | VTESGCKECEELEEKNIKEFLQSFVHIVAMFINTS |
| SEQ ID NO: | IL-15Rαsushi-IL-15 | ITCPPPMSVEHADIWVKSYSLYSRERYICNSGFKRKAGTS |
| 308 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVENLIILANNSLSSNGNVTESGCKECEELEEKNIKEF |
| | | LQSFVHIVAMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0359

| | | |
|---|---|---|
| SEQ ID NO: | Heavy chain (VH- | QVQLVQSGAEVKKPGSSVKVSCKASGHDFRLTYIHWVR |
| 289 | CH)-Hole | QAPGQGLEWMGRVSAGSGNVKYNEKFKGRVTITADTS |
| | | TSTAYMELSSLRSEDTAVYYCAVSYYSYDVLDYWGQG |
| | | TTVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKRVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSV |
| | | LTVLHQDWLNGKEYKCKVSNKALPAPIEKTISKAKGQP |
| | | REPQVCTLPPSREEMTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSP |
| SEQ ID NO: | Light chain (VL- | DIQMTQSPSFLSASVGDRVTITCKASQNVADRVVWHQQ |
| 298 | CL) | KPGKAPKALIYAASYLQSGVPSRFSGSGSGTEFTLTISSL |
| | | QPEDFATYFCQQFKSYPLTFGQGTKLEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: | IL-15Rαsushi-IL-15 | ITCPPPMSVEHADIWVKSYSLYSRERYICNSGFKRKAGTS |
| 304 | Heavy Chain | SLTECVLNKATNVAHWTTPSLKCIRDPALVHQRPAPPSG |
| | Constant Domain | GSGGGGSGGGSGGGGSGGGNWVNVISDLKKIEDLIQSM |
| | (Fc Region)- | HIDATLYTESDVHPSCKVTAMKCFLLELQVISLESGDASI |
| | Knob | HDTVENLIILANNSLSSNGNVTESGCKECEELEEKNIKEF |
| | | LQSFVHIVAMFINTSGGGGSGGGGSDKTHTCPPCPAPEL |
| | | LGGPSVFLFPPKPKDTLMISRTPEVTCVVVDVSHEDPEV |
| | | KFNWYVDGVEVHNAKTKPREEQYASTYRVVSVLTVLH |
| | | QDWLNGKEYKCKVSNKALPAPIEKTISKAKGQPREPQV |
| | | YTLPPCREEMTKNQVSLWCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | AASYLQS |
| 299 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 300 | | KPGKAPKALIYAASYLQSGVPSRFSGSGSGTEFTLTISSL |
| | | QPEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 307 | | MKCFLLELQVISLESGDASIHDTVENLIILANNSLSSNGN |
| | | VTESGCKECEELEEKNIKEFLQSFVHIVAMFINTS |
| SEQ ID NO: | IL-15Rαsushi-IL-15 | ITCPPPMSVEHADIWVKSYSLYSRERYICNSGFKRKAGTS |
| 308 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVENLIILANNSLSSNGNVTESGCKECEELEEKNIKEF |
| | | LQSFVHIVAMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0355

| | | |
|---|---|---|
| SEQ ID NO: | Heavy chain (VH- | QVQLVQSGAEVKKPGSSVKVSCKASGHDFRLTYIHWV |
| 289 | CH)-Hole | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYASTYRV |
| | | VSVLTVLHQDWLNGKEYKCKVSNKALPAPIEKTISKAK |
| | | GQPREPQVCTLPPSREEMTKNQVSLSCAVKGFYPSDIAV |
| | | EWESNGQPENNYKTTPPVLDSDGSFFLVSKLTVDKSRW |
| | | QQGNVFSCSVMHEALHNHYTQKSLSLSP |
| SEQ ID NO: | Light chain (VL- | DIQMTQSPSFLSASVGDRVTITCKASQNVADRVVWHQQ |
| 301 | CL) | KPGKAPKALIFAASYLQSGVPSRFSGSGSGTEFTLTISSLQ |
| | | PEDFATYFCQQFKSYPLTFGQGTKLEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| SEQ ID NO: | IL-15Rαsushi-IL-15 | ITCPPPMSVEHADIWVKSYSLYSRERYICNSGFKRKAGTS |
| 304 | Heavy Chain | SLTECVLNKATNVAHWTTPSLKCIRDPALVHQRPAPPSG |
| | Constant Domain | GSGGGGSGGGSGGGGSGGGNWVNVISDLKKIEDLIQSM |
| | (Fc Region)- | HIDATLYTESDVHPSCKVTAMKCFLLELQVISLESGDASI |
| | Knob | HDTVENLIILANNSLSSNGNVTESGCKECEELEEKNIKEF |
| | | LQSFVHIVAMFINTSGGGGSGGGGSDKTHTCPPCPAPEL |
| | | LGGPSVFLFPPKPKDTLMISRTPEVTCVVVDVSHEDPEV |
| | | KFNWYVDGVEVHNAKTKPREEQYASTYRVVSVLTVLH |
| | | QDWLNGKEYKCKVSNKALPAPIEKTISKAKGQPREPQV |
| | | YTLPPCREEMTKNQVSLWCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | AASYLQS |
| 299 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 303 | | KPGKAPKALIFAASYLQSGVPSRFSGSGSGTEFTLTISSLQ |
| | | PEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 307 | | MKCFLLELQVISLESGDASIHDTVENLIILANNSLSSNGN |
| | | VTESGCKECEELEEKNIKEFLQSFVHIVAMFINTS |
| SEQ ID NO: | IL-15Rαsushi-IL-15 | ITCPPPMSVEHADIWVKSYSLYSRERYICNSGFKRKAGTS |
| 308 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVENLIILANNSLSSNGNVTESGCKECEELEEKNIKEF |
| | | LQSFVHIVAMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0308

| | | |
|---|---|---|
| SEQ ID NO: | Heavy chain (VH- | QVQLVQSGAEVKKPGSSVKVSCKASGHDFRLTYIHWVR |
| 289 | CH)-Hole | QAPGQGLEWMGRVSAGSGNVKYNEKFKGRVTITADTS |
| | | TSTAYMELSSLRSEDTAVYYCAVSYYSYDVLDYWGQG |
| | | TTVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKRVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSV |
| | | LTVLHQDWLNGKEYKCKVSNKALPAPIEKTISKAKGQP |
| | | REPQVCTLPPSREEMTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSP |
| SEQ ID NO: | Light chain (VL- | DIQMTQSPSFLSASVGDRVTITCKASQNVADRVVWHQQ |
| 239 | CL) | KPGKAPKALIYSSSHRYKGVPSRFSGSGSGTEFTLTISSLQ |
| | | PEDFATYFCQQFKSYPLTFGQGTKLEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| SEQ ID NO: | IL-15Rαsushi-IL-15 | ITCPPPMSVEHADIWVKSYSLYSRERYICNSGFKRKAGTS |
| 309 | Heavy Chain | SLTECVLNKATNVAHWTTPSLKCIRDPALVHQRPAPPSG |
| | Constant Domain | GSGGGGSGGGSGGGGSGGGNWVNVISDLKKIEDLIQSM |
| | (Fc Region)- | HIDATLYTESDVHPSCKVTAMKCFLLELQVISLESGDASI |
| | Knob | HDTVEQLIILANNSLSSNGNVTESGCKECEELEEKNIKEF |
| | | LQSFVHIVQMFINTSGGGGSGGGGSDKTHTCPPCPAPEL |
| | | LGGPSVFLFPPKPKDTLMISRTPEVTCVVVDVSHEDPEV |
| | | KFNWYVDGVEVHNAKTKPREEQYASTYRVVSVLTVLH |
| | | QDWLNGKEYKCKVSNKALPAPIEKTISKAKGQPREPQV |
| | | YTLPPCREEMTKNQVSLWCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | SSSHRYK |
| 167 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 173 | | KPGKAPKALIYSSSHRYKGVPSRFSGSGSGTEFTLTISSLQ |
| | | PEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 310 | | MKCFLLELQVISLESGDASIHDTVEQLIILANNSLSSNGN |
| | | VTESGCKECEELEEKNIKEFLQSFVHIVQMFINTS |
| SEQ ID NO: | IL-15Rαsushi-IL-15 | ITCPPPMSVEHADIWVKSYSLYSRERYICNSGFKRKAGTS |
| 311 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVEQLIILANNSLSSNGNVTESGCKECEELEEKNIKEF |
| | | LQSFVHIVQMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0360

| | | |
|---|---|---|
| SEQ ID NO: | Heavy chain (VH- | QVQLVQSGAEVKKPGSSVKVSCKASGHDFRLTYIHWVR |
| 289 | CH)-Hole | QAPGQGLEWMGRVSAGSGNVKYNEKFKGRVTITADTS |
| | | TSTAYMELSSLRSEDTAVYYCAVSYYSYDVLDYWGQG |
| | | TTVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKRVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSV |
| | | LTVLHQDWLNGKEYKCKVSNKALPAPIEKTISKAKGQP |
| | | REPQVCTLPPSREEMTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSP |
| SEQ ID NO: | Light chain (VL- | DIQMTQSPSFLSASVGDRVTITCKASQNVADRVVWHQQ |
| 298 | CL) | KPGKAPKALIYAASYLQSGVPSRFSGSGSGTEFTLTISSL |
| | | QPEDFATYFCQQFKSYPLTFGQGTKLEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: | IL-15Rasushi-IL-15 | ITCPPPMSVEHADIWVKSYSLYSRERYICNSGFKRKAGTS |
| 309 | Heavy Chain | SLTECVLNKATNVAHWTTPSLKCIRDPALVHQRPAPPSG |
| | Constant Domain | GSGGGGSGGGSGGGGSGGGNWVNVISDLKKIEDLIQSM |
| | (Fc Region)- | HIDATLYTESDVHPSCKVTAMKCFLLELQVISLESGDASI |
| | Knob | HDTVEQLIILANNSLSSNGNVTESGCKECEELEEKNIKEF |
| | | LQSFVHIVQMFINTSGGGGSGGGGSDKTHTCPPCPAPEL |
| | | LGGPSVFLFPPKPKDTLMISRTPEVTCVVVDVSHEDPEV |
| | | KFNWYVDGVEVHNAKTKPREEQYASTYRVVSVLTVLH |
| | | QDWLNGKEYKCKVSNKALPAPIEKTISKAKGQPREPQV |
| | | YTLPPCREEMTKNQVSLWCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | AASYLQS |
| 299 | | |
| SEQ ID NO: 8 | LC CDR3 | QQFKSYPLT |
| SEQ ID NO: | VH | QVQLVQSGAEVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 300 | | KPGKAPKALIYAASYLQSGVPSRFSGSGSGTEFTLTISSL |
| | | QPEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 310 | | MKCFLLELQVISLESGDASIHDTVEQLIILANNSLSSNGN |
| | | VTESGCKECEELEEKNIKEFLQSFVHIVQMFINTS |
| SEQ ID NO: | IL-15Rαsushi-IL-15 | ITCPPPMSVEHADIWVKSYSLYSRERYICNSGFKRKAGTS |
| 311 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVEQLIILANNSLSSNGNVTESGCKECEELEEKNIKEF |
| | | LQSFVHIVQMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BMM0357

| | | |
|---|---|---|
| SEQ ID NO: | Heavy chain (VH- | QVQLVQSGAEVKKPGSSVKVSCKASGHDFRLTYIHWVR |
| 289 | CH)-Hole | QAPGQGLEWMGRVSAGSGNVKYNEKFKGRVTITADTS |
| | | TSTAYMELSSLRSEDTAVYYCAVSYYSYDVLDYWGQG |
| | | TTVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKRVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSV |
| | | LTVLHQDWLNGKEYKCKVSNKALPAPIEKTISKAKGQP |
| | | REPQVCTLPPSREEMTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSP |
| SEQ ID NO: | Light chain (VL- | DIQMTQSPSFLSASVGDRVTITCKASQNVADRVVWHQQ |
| 301 | CL) | KPGKAPKALIFAASYLQSGVPSRFSGSGSGTEFTLTISSLQ |
| | | PEDFATYFCQQFKSYPLTFGQGTKLEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| SEQ ID NO: | IL-15Rαsushi-IL-15 | ITCPPPMSVEHADIWVKSYSLYSRERYICNSGFKRKAGTS |
| 309 | Heavy Chain | SLTECVLNKATNVAHWTTPSLKCIRDPALVHQRPAPPSG |
| | Constant Domain | GSGGGGSGGGSGGGGSGGGNWVNVISDLKKIEDLIQSM |
| | (Fc Region)- | HIDATLYTESDVHPSCKVTAMKCFLLELQVISLESGDASI |
| | Knob | HDTVEQLIILANNSLSSNGNVTESGCKECEELEEKNIKEF |
| | | LQSFVHIVQMFINTSGGGGSGGGGSDKTHTCPPCPAPEL |
| | | LGGPSVFLFPPKPKDTLMISRTPEVTCVVVDVSHEDPEV |
| | | KFNWYVDGVEVHNAKTKPREEQYASTYRVVSVLTVLH |
| | | QDWLNGKEYKCKVSNKALPAPIEKTISKAKGQPREPQV |
| | | YTLPPCREEMTKNQVSLWCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: | HC CDR1 | GHDFRLTYIH |
| 164 | | |
| SEQ ID NO: | HC CDR1 | LTYIH |
| 229 | | |
| SEQ ID NO: | HC CDR1 | FRLTYIH |
| 234 | | |
| SEQ ID NO: | HC CDR2 | RVSAGSGNVKYNEKFKG |
| 165 | | |
| SEQ ID NO: 5 | HC CDR3 | SYYSYDVLDY |
| SEQ ID NO: | LC CDR1 | KASQNVADRVV |
| 171 | | |
| SEQ ID NO: | LC CDR2 | AASYLQS |
| 299 | | |
| SEQ ID NO: 8 | LC CDR3 | QQFKSYPLT |
| SEQ ID NO: | VH | QVQLVQSGARVKKPGSSVKVSCKASGHDFRLTYIHWVR |
| 168 | | QAPGQGLEWMGRVSAGSGNVKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVADRVVWHQQ |
| 303 | | KPGKAPKALIFAASYLQSGVPSRFSGSGSGTEFTLTISSLQ |
| | | PEDFATYFCQQFKSYPLTFGQGTKLEIK |
| SEQ ID NO: | Heavy chain | ASTKGPSVFPLAPSSKSTSGGTAALGCLVKDYFPEPVTVS |
| 293 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSP |
| SEQ ID NO: | Heavy chain hinge- | DKTHTCPPCPAPELLGGPSVFLFPPKPKDTLMISRTPEVT |
| 294 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSP |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-15Rαsushi (AA | ITCPPPMSVEHADIWVKSYSLYSRERYICNSGFKRKAGTS |
| 295 | 31-108 of IL- | SLTECVLNKATNVAHWTTPSLKCIRDPALVHQRPAPPS |
| | 15Ralpha) | |
| SEQ ID NO: | Linker | GGSGGGGSGGGSGGGGSGGG |
| 296 | | |
| SEQ ID NO: | AA 49-162 of IL-15 | NWVNVISDLKKIEDLIQSMHIDATLYTESDVHPSCKVTA |
| 310 | | MKCFLLELQVISLESGDASIHDTVEQLIILANNSLSSNGN |
| | | VTESGCKECEELEEKNIKEFLQSFVHIVQMFINTS |
| SEQ ID NO: | IL-15Rαsushi-IL-15 | ITCPPPMSVEHADIWVKSYSLYSRERYICNSGFKRKAGTS |
| 311 | (AA 31-108 of IL- | SLTECVLNKATNVAHWTTPSLKCIRDPALVHQRPAPPSG |
| | 15Ralpha-Linker- | GSGGGGSGGGSGGGGSGGGNWVNVISDLKKIEDLIQSM |
| | AA 31-108 of IL- | HIDATLYTESDVHPSCKVTAMKCFLLELQVISLESGDASI |
| | 15Ralpha) | HDTVEQLIILANNSLSSNGNVTESGCKECEELEEKNIKEF |
| | | LQSFVHIVQMFINTS |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

TABLE 11
Exemplary binding affinities of exemplary molecule
comprising an antigen binding domain that binds
to a T cell receptor beta variable region (TCRβV).

| Exemplary molecule | Affinity (K_{D}) |
|---|---|
| BMM0306 | Affinity of TCRβV arm = 1.2 nM |
| | Affinity of IL-15Rαsushi-IL-15 arm = 0.2 nM |
| BMM0354 | Affinity of TCRβV arm = 67 nM |
| | Affinity of IL-15Rαsushi-IL-15 arm = 0.2 nM |
| BMM0356 | Affinity of TCRβV arm = 681 nM |
| | Affinity of IL-15Rαsushi-IL-15 arm = 0.2 nM |
| BMM0307 | Affinity of TCRβV arm = 1.2 nM |
| | Affinity of IL-15Rαsushi-IL-15 arm = 3.2 nM |
| BMM0359 | Affinity of TCRβV arm = 67 nM |
| | Affinity of IL-15Rαsushi-IL-15 arm = 3.2 nM |
| BMM0355 | Affinity of TCRβV arm = 681 nM |
| | Affinity of IL-15Rαsushi-IL-15 arm = 3.2 nM |
| BMM0308 | Affinity of TCRβV arm = 1.2 nM |
| | Affinity of IL-15Rαsushi-IL-15 arm = 40 nM |
| BMM0360 | Affinity of TCRβV arm = 67 nM |
| | Affinity of IL-15Rαsushi-IL-15 arm = 40 nM |
| BMM0357 | Affinity of TCRβV arm = 681 nM |
| | Affinity of IL-15Rαsushi-IL-15 arm = 40 nM |

TABLE 12
Amino acid sequences for additional TCRβV antibodies. Amino acid and nucleotide
sequences for murine and humanized antibody molecules which bind to various TCRVB
families are disclosed. The amino acid the heavy and light chain CDRs, and the amino
acid and nucleotide sequences of the heavy and light chain variable regions, and the
heavy and light chains are shown. Antibodies disclosed in the table include, IMMU222.
IMMU 222 binds human TCRβV 6-5, TCRβV 6-6, or TCRβV 6-9 (TCRβV13.1 per old nomenclature).
IMMU222 (murine) also referred to as BJ1461; or Antibody I
Binds to human TCRVβ 6-5, 6-6, 6-9

| | | |
|---|---|---|
| SEQ ID NO: 314 | HC CDR1 (Kabat) | SYAMS |
| SEQ ID NO: 315 | HC CDR2 (Kabat) | HISNGGDYIYYADTVKG |
| SEQ ID NO: 316 | HC CDR3 (Kabat) | PSYYSDPWFFDV |
| SEQ ID NO: 317 | HC CDR1 (Chothia) | GFTFRSY |
| SEQ ID NO: 318 | HC CDR2 (Chothia) | SNGGDY |
| SEQ ID NO: 316 | HC CDR3 (Chothia) | PSYYSDPWFFDV |
| SEQ ID NO: 319 | HC CDR1 (Combined) | GFTFRSYAMS |
| SEQ ID NO: 315 | HC CDR2 (Combined) | HISNGGDYIYYADTVKG |
| SEQ ID NO: 316 | HC CDR3(Combined) | PSYYSDPWFFDV |
| SEQ ID NO: 320 | LC CDR1 (Kabat) | SAGSSVSFMH |
| SEQ ID NO: 321 | LC CDR2 (Kabat) | DTSKLAS |
| SEQ ID NO: 322 | LC CDR3 (Kabat) | LQGSGFPLT |
| SEQ ID NO: 323 | LC CDR1 (Chothia) | GSSVSF |
| SEQ ID NO: 321 | LC CDR2 (Chothia) | DTSKLAS |
| SEQ ID NO: 322 | LC CDR3 (Chothia) | LQGSGFPLT |
| SEQ ID NO: 320 | LC CDR1 (Combined) | SAGSSVSFMH |
| SEQ ID NO: 321 | LC CDR2 (Combined) | DTSKLAS |
| SEQ ID NO: 322 | LC CDR3(Combined) | LQGSGFPLT |
| SEQ ID NO: 324 | VL | ENVLTQSPAIMSASPGEKVTMTCSAGSSVSFMHWYQQK |
| | | SSTSPKLWIYDTSKLASGVPGRFSGSGSGNSFSLTISSME |
| | | AEDVAIYYCLQGSGFPLTFGSGTKLEIK |
| SEQ ID NO: 325 | VH | DVKLVESGEGLVKPGGSLKLSCAASGFTFRSYAMSWVR |
| | | QTPEKRLEWVAHISNGGDYIYYADTVKGRFTISRDNAR |
| | | NTLYLQMSSLKSEDTAMYYCTRPSYYSDPWFFDVWGT |
| | | GTTVTVSS |

VH for IMMU222 (humanized) also referred to as Antibody I-H
Binds to human TCRVβ 6-5, 6-6, 6-9

| | | |
|---|---|---|
| SEQ ID NO: 326 | VH-1 | EVQLVESGGGLVQPGGSLRLSCAASGFTFRSYAMSWVR |
| | | QAPGKGLEWVAHISNGGDYIYYADTVKGRFTISRDNAK |
| | | NSLYLQMNSLRAEDTAVYYCTRPSYYSDPWFFDVWGQ |
| | | GTTVTVSS |
| SEQ ID NO: 327 | VH-2 | QVQLVESGGGVVQPGRSLRLSCAASGFTFRSYAMSWVR |
| | | QAPGKGLEWVAHISNGGDYIYYADTVKGRFTISRDNSK |
| | | NTLYLQMSSLRAEDTAVYYCTRPSYYSDPWFFDVWGQ |
| | | GTTVTVSS |
| SEQ ID NO: 328 | VH-3 | EVQLVESGGGLVQPGGSLRLSCAASGFTFRSYAMSWVR |
| | | QAPGKGLEWVAHISNGGDYIYYADTVKGRFTISRDNSK |
| | | NTLYLQMNSLRAEDTAVYYCTRPSYYSDPWFFDVWGQ |
| | | GTTVTVSS |
| SEQ ID NO: 329 | VH-4 | QVQLVQSGSELKKPGASVKVSCKASGFTFRSYAMSWVR |
| | | QAPGQGLEWVAHISNGGDYIYYADTVKGRFVISRDNSV |
| | | NTLYLQISSLKAEDTAVYYCTRPSYYSDPWFFDVWGQG |
| | | TTVTVSS |
| SEQ ID NO: 330 | VH-5 | QVQLVQSGAEVKKPGASVKVSCKASGFTFRSYAMSWV |
| | | RQAPGQRLEWVAHISNGGDYIYYADTVKGRFTITRDNS |
| | | ANTLYMELSSLRSEDTAVYYCTRPSYYSDPWFFDVWGQ |
| | | GTTVTVSS |

VL for IMMU222 (humanized)) also referred to as Antibody I-H
Binds to human TCRVβ 6-5, 6-6, 6-9

| | | |
|---|---|---|
| SEQ ID NO: 331 | VL-1 | ENVLTQSPATLSLSPGERATLSCSAGSSVSFMHWYQQKP |
| | | GQAPKLLIYDTSKLASGIPARFSGSGSGNDFTLTISSLEPE |
| | | DFAVYYCLQGSGFPLTFGQGTKLEIK |
| SEQ ID NO: 332 | VL-2 | ENVLTQSPDFQSVTPKEKVTITCSAGSSVSFMHWYQQKP |
| | | DQSPKLLIYDTSKLASGVPSRFSGSGSGNDFTLTINSLEA |
| | | EDAATYYCLQGSGFPLTFGQGTKLEIK |
| SEQ ID NO: 333 | VL-3 | DNQLTQSPSSLSASVGDRVTITCSAGSSVSFMHWYQQKP |
| | | GKVPKLLIYDTSKLASGVPSRFSGSGSGNDFTLTISSLQP |
| | | EDVATYYCLQGSGFPLTFGQGTKLEIK |
| SEQ ID NO: 334 | VL-4 | ANQLTQSPSSLSASVGDRVTITCSAGSSVSFMHWYQQKP |
| | | GKAPKLLIYDTSKLASGVPSRFSGSGSGNDFTLTISSLQP |
| | | EDFATYYCLQGSGFPLTFGQGTKLEIK |
| SEQ ID NO: 335 | VL-5 | DNVLTQSPDSLAVSLGERATINCSAGSSVSFMHWYQQK |
| | | PGQPPKLLIYDTSKLASGVPDRFSGSGSGNDFTLTISSLQ |
| | | AEDVAVYYCLQGSGFPLTFGQGTKLEIK |

TABLE 13
Exemplary multispecific molecule that comprise a first domain that binds to a first
target molecule, wherein the exemplary target molecule comprises TCRβ V20 (e.g., TCRVB
20-1). Exemplary anti-TCR V20 antibodies include, but are not limited to, BLM0274 and
BLM0276, which binds to human TCRβV 20-1. CDRs are underlined.
BLM0274, also referred to as BJ1325 or BJ1329
Binds to human TCRVβ 20-1

| | | |
|---|---|---|
| SEQ ID NO: 336 | Heavy chain (VH- | QVQLVQSGAEVKKPGASVKVSCKASGFNIKSAYMHWV |
| | CH) | RQAPGQEPGCMGRIDPATGKTKYAPKFQARVTMTADT |
| | | SINTAYTELSSLRSEDTATYYCARSLNWDYGLDYWGQ |
| | | GTLVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYASTYRV |
| | | VSVLTVLHQDWLNGKEYKCKVSNKALPAPIEKTISKAK |
| | | GQPREPQVCTLPPSREEMTKNQVSLSCAVKGFYPSDIAV |
| | | EWESNGQPENNYKTTPPVLDSDGSFFLVSKLTVDKSRW |
| | | QQGNVFSCSVMHEALHNHYTQKSLSLSPGK |
| SEQ ID NO: 337 | Light chain (VL- | EIVLTQSPATLSLSPGERATLSCRASKSVSILGTHLIHWY |
| | CL) | QQKPGLAPRLLIYAASNLESGIPDRFSGSGSETDFTLTIS |
| | | RLEPEDFAVYFCQQSIEDPWTFGGGTKVEIKRTVAAPSV |
| | | FIFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |
| SEQ ID NO: 338 | IL-2 C125A-Heavy | APTSSSTKKTQLQLEHLLLDLQMILNGINNYKNPKLTR |
| | Chain Constant | MLTFKFYMPKKATELKHLQCLEEELKPLEEVLNLAQSK |
| | Domain (Fc Region) | NFHLRPRDLISNINVIVLELKGSETTFMCEYADETATIVE |
| | | FLNRWITFAQSIISTLTGGGGSGGGGSDKTHTCPPCPAPE |
| | | LLGGPSVFLFPPKPKDTLMISRTPEVTCVVVDVSHEDPE |
| | | VKFNWYVDGVEVHNAKTKPREEQYASTYRVVSVLTVL |
| | | HQDWLNGKEYKCKVSNKALPAPIEKTISKAKGQPREPQ |
| | | VYTLPPCREEMTKNQVSLWCLVKGFYPSDIAVEWESN |
| | | GQPENNYKTTPPVLDSDGSFFLYSKLTVDKSRWQQGN |
| | | VFSCSVMHEALHNHYTQKSLSLSPGK |
| SEQ ID NO: 339 | HC CDR1 | GFNIKSAYMH |
| SEQ ID NO: 340 | HC CDR2 | RIDPATGKTKYAPKFQA |
| SEQ ID NO: 341 | HC CDR3 | SLNWDYGLDY |
| SEQ ID NO: 342 | LC CDR1 | RASKSVSILGTHLIH |
| SEQ ID NO: 343 | LC CDR2 | AASNLES |
| SEQ ID NO: 344 | LC CDR3 | QQSIEDPWT |
| SEQ ID NO: | VH | QVQLVQSGAEVKKPGASVKVSCKASGFNIKSAYMHWV |
| 345 | | RQAPGQEPGCMGRIDPATGKTKYAPKFQARVTMTADTS |
| | | INTAYTELSSLRSEDTATYYCARSLNWDYGLDYWGQGT |
| | | LVTVSS |
| SEQ ID NO: | VL | EIVLTQSPATLSLSPGERATLSCRASKSVSILGTHLIHWYQ |
| 346 | | QKPGLAPRLLIYAASNLESGIPDRFSGSGSETDFTLTISRL |
| | | EPEDFAVYFCQQSIEDPWTFGGGTKVEIK |
| SEQ ID NO: | Heavy chain | ASTKGPSVFPLAPSSKSTSGGTAALGCLVKDYFPEPVTVS |
| 207 | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSPGK |
| SEQ ID NO: | Heavy chain hinge- | DKTHTCPPCPAPELLGGPSVFLFPPKPKDTLMISRTPEVT |
| 347 | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSPGK |
| SEQ ID NO: | Light chain constant | RTVAAPSVFIFPPSDEQLKSGTASVVCLLNNFYPREAKV |
| 203 | region | QWKVDNALQSGNSQESVTEQDSKDSTYSLSSTLTLSKA |
| | | DYEKHKVYACEVTHQGLSSPVTKSFNRGEC |
| SEQ ID NO: | IL-2 C125A | APTSSSTKKTQLQLEHLLLDLQMILNGINNYKNPKLTRM |
| 242 | | LTFKFYMPKKATELKHLQCLEEELKPLEEVLNLAQSKNF |
| | | HLRPRDLISNINVIVLELKGSETTFMCEYADETATIVEFLN |
| | | RWITFAQSIISTLT |
| SEQ ID NO: | Linker | GGGGSGGGGS |
| 237 | | |
| SEQ ID NO: | Heavy chain hinge- | DKTHTCPPCPAPELLGGPSVFLFPPKPKDTLMISRTPEVT |
| 206 | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BLM0276, also referred to as BJ1324, BJ1330
Binds to human TCRVβ 20-1

| | | |
|---|---|---|
| SEQ ID NO: 348 | Heavy chain (VH- | QVQLVQSGAEVKKPGASVKVSCKASGFNIKSAYMHWV |
| | CH | RQAPGQGLEWMGRIDPATGKTKYAPKFQARVTMTADT |
| | | STNTAYMELSSLRSEDTAVYYCARSLNWDYGLDYWG |
| | | QGTLVTVSSASTKGPSVFPLAPSSKSTSGGTAALGCLVK |
| | | DYFPEPVTVSWNSGALTSGVHTFPAVLQSSGLYSLSSV |
| | | VTVPSSSLGTQTYICNVNHKPSNTKVDKRVEPKSCDKT |
| | | HTCPPCPAPELLGGPSVFLFPPKPKDTLMISRTPEVTCVV |
| | | VDVSHEDPEVKFNWYVDGVEVHNAKTKPREEQYASTY |
| | | RVVSVLTVLHQDWLNGKEYKCKVSNKALPAPIEKTISK |
| | | AKGQPREPQVCTLPPSREEMTKNQVSLSCAVKGFYPSDI |
| | | AVEWESNGQPENNYKTTPPVLDSDGSFFLVSKLTVDKS |
| | | RWQQGNVFSCSVMHEALHNHYTQKSLSLSPGK |
| SEQ ID NO: 349 | Light chain (VL-CL) | EIVLTQSPGTLSLSPGERATLSCRASKSVSILGTHLIHWY |
| | | QQKPGQAPRLLIYAASNLESGIPDRFSGSGSETDFTLTIS |
| | | RLEPEDFAVYFCQQSIEDPWTFGGGTKVEIKRTVAAPSV |
| | | FIFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |
| SEQ ID NO: 338 | IL-2 C125A-Heavy | APTSSSTKKTQLQLEHLLLDLQMILNGINNYKNPKLTR |
| | Chain Constant | MLTFKFYMPKKATELKHLQCLEEELKPLEEVLNLAQSK |
| | Domain (Fc Region) | NFHLRPRDLISNINVIVLELKGSETTFMCEYADETATIVE |
| | | FLNRWITFAQSIISTLTGGGGSGGGGSDKTHTCPPCPAPE |
| | | LLGGPSVFLFPPKPKDTLMISRTPEVTCVVVDVSHEDPE |
| | | VKFNWYVDGVEVHNAKTKPREEQYASTYRVVSVLTVL |
| | | HQDWLNGKEYKCKVSNKALPAPIEKTISKAKGQPREPQ |
| | | VYTLPPCREEMTKNQVSLWCLVKGFYPSDIAVEWESN |
| | | GQPENNYKTTPPVLDSDGSFFLYSKLTVDKSRWQQGN |
| | | VFSCSVMHEALHNHYTQKSLSLSPGK |
| SEQ ID NO: 339 | HC CDR1 | GFNIKSAYMH |
| SEQ ID NO: 340 | HC CDR2 | RIDPATGKTKYAPKFQA |
| SEQ ID NO: 341 | HC CDR3 | SLNWDYGLDY |
| SEQ ID NO: 342 | LC CDR1 | RASKSVSILGTHLIH |
| SEQ ID NO: 343 | LC CDR2 | AASNLES |
| SEQ ID NO: 344 | LC CDR3 | QQSIEDPWT |
| SEQ ID NO: 350 | VH | QVQLVQSGAEVKKPGASVKVSCKASGFNIKSAYMHWV |
| | | RQAPGQGLEWMGRIDPATGKTKYAPKFQARVTMTADT |
| | | STNTAYMELSSLRSEDTAVYYCARSLNWDYGLDYWGQ |
| | | GTLVTVSS |
| SEQ ID NO: 351 | VL | EIVLTQSPGTLSLSPGERATLSCRASKSVSILGTHLIHWYQ |
| | | QKPGQAPRLLIYAASNLESGIPDRFSGSGSETDFTLTISRL |
| | | EPEDFAVYFCQQSIEDPWTFGGGTKVEIK |
| SEQ ID NO: 207 | Heavy chain | ASTKGPSVFPLAPSSKSTSGGTAALGCLVKDYFPEPVTVS |
| | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSPGK |
| SEQ ID NO: 347 | Heavy chain hinge- | DKTHTCPPCPAPELLGGPSVFLFPPKPKDTLMISRTPEVT |
| | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSPGK |
| SEQ ID NO: 203 | Light chain constant | RTVAAPSVFIFPPSDEQLKSGTASVVCLLNNFYPREAKV |
| | region | QWKVDNALQSGNSQESVTEQDSKDSTYSLSSTLTLSKA |
| | | DYEKHKVYACEVTHQGLSSPVTKSFNRGEC |
| SEQ ID NO: 242 | IL-2 C125A | APTSSSTKKTQLQLEHLLLDLQMILNGINNYKNPKLTRM |
| | | LTFKFYMPKKATELKHLQCLEEELKPLEEVLNLAQSKNF |
| | | HLRPRDLISNINVIVLELKGSETTFMCEYADETATIVEFLN |
| | | RWITFAQSIISTLT |
| SEQ ID NO: 237 | Linker | GGGGSGGGGS |
| SEQ ID NO: 206 | Heavy chain hinge- | DKTHTCPPCPAPELLGGPSVFLFPPKPKDTLMISRTPEVT |
| | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGK |

BLM0000

| | | |
|---|---|---|
| SEQ ID NO: 348 | Heavy chain (VH- | QVQLVQSGAEVKKPGASVKVSCKASGFNIKSAYMHWV |
| | CH) | RQAPGQGLEWMGRIDPATGKTKYAPKFQARVTMTADT |
| | | STNTAYMELSSLRSEDTAVYYCARSLNWDYGLDYWG |
| | | QGTLVTVSSASTKGPSVFPLAPSSKSTSGGTAALGCLVK |
| | | DYFPEPVTVSWNSGALTSGVHTFPAVLQSSGLYSLSSV |
| | | VTVPSSSLGTQTYICNVNHKPSNTKVDKRVEPKSCDKT |
| | | HTCPPCPAPELLGGPSVFLFPPKPKDTLMISRTPEVTCVV |
| | | VDVSHEDPEVKFNWYVDGVEVHNAKTKPREEQYASTY |
| | | RVVSVLTVLHQDWLNGKEYKCKVSNKALPAPIEKTISK |
| | | AKGQPREPQVCTLPPSREEMTKNQVSLSCAVKGFYPSDI |
| | | AVEWESNGQPENNYKTTPPVLDSDGSFFLVSKLTVDKS |
| | | RWQQGNVFSCSVMHEALHNHYTQKSLSLSPGK |
| SEQ ID NO: 349 | Light chain (VL-CL) | EIVLTQSPGTLSLSPGERATLSCRASKSVSILGTHLIHWY |
| | | QQKPGQAPRLLIYAASNLESGIPDRFSGSGSETDFTLTIS |
| | | RLEPEDFAVYFCQQSIEDPWTFGGGTKVEIKRTVAAPSV |
| | | FIFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |
| SEQ ID NO: 352 | IL-2 C125A-Heavy | APTSSSTKKTQLQLEHLLLDLQMILNGINNYKNPKLTR |
| | Chain Constant | MLTFKFYMPKKATELKHLQCLEEELKPLEEVLNLAQSK |
| | Domain (Fc Region) | NFHLRPRDLISNINVIVLELKGSETTFMCEYADETATIVE |
| | | FLNRWITFAQSIISTLTGGGGSGGGGSDKTHTCPPCPAPE |
| | | LLGGPSVFLFPPKPKDTLMISRTPEVTCVVVDVSHEDPE |
| | | VKFNWYVDGVEVHNAKTKPREEQYASTYRVVSVLTVL |
| | | HQDWLNGKEYKCKVSNKALPAPIEKTISKAKGQPREPQ |
| | | VYTLPPCREEMTKNQVSLWCLVKGFYPSDIAVEWESN |
| | | GQPENNYKTTPPVLDSDGSFFLYSKLTVDKSRWQQGN |
| | | VFSCSVMHEALHNHYTQKSLSLSP |
| SEQ ID NO: 339 | HC CDR1 | GFNIKSAYMH |
| SEQ ID NO: 340 | HC CDR2 | RIDPATGKTKYAPKFQA |
| SEQ ID NO: 341 | HC CDR3 | SLNWDYGLDY |
| SEQ ID NO: 342 | LC CDR1 | RASKSVSILGTHLIH |
| SEQ ID NO: 343 | LC CDR2 | AASNLES |
| SEQ ID NO: 344 | LC CDR3 | QQSIEDPWT |
| SEQ ID NO: 350 | VH | QVQLVQSGAEVKKPGASVKVSCKASGFNIKSAYMHWV |
| | | RQAPGQGLEWMGRIDPATGKTKYAPKFQARVTMTADT |
| | | STNTAYMELSSLRSEDTAVYYCARSLNWDYGLDYWGQ |
| | | GTLVTVSS |
| SEQ ID NO: 341 | VL | EIVLTQSPGTLSLSPGERATLSCRASKSVSILGTHLIHWYQ |
| | | QKPGQAPRLLIYAASNLESGIPDRFSGSGSETDFTLTISRL |
| | | EPEDFAVYFCQQSIEDPWTFGGGTKVEIK |
| SEQ ID NO: 207 | Heavy chain | ASTKGPSVFPLAPSSKSTSGGTAALGCLVKDYFPEPVTVS |
| | constant region | WNSGALTSGVHTFPAVLQSSGLYSLSSVVTVPSSSLGTQ |
| | (CH1-CH2-CH3) | TYICNVNHKPSNTKVDKRVEPKSCDKTHTCPPCPAPELL |
| | Hole | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVCTL |
| | | PPSREEMTKNQVSLSCAVKGFYPSDIAVEWESNGQPENN |
| | | YKTTPPVLDSDGSFFLVSKLTVDKSRWQQGNVFSCSVM |
| | | HEALHNHYTQKSLSLSPGK |
| SEQ ID NO: 347 | Heavy chain hinge- | DKTHTCPPCPAPELLGGPSVFLFPPKPKDTLMISRTPEVT |
| | Fc region (CH2- | CVVVDVSHEDPEVKFNWYVDGVEVHNAKTKPREEQYA |
| | CH3) Hole | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSPGK |
| SEQ ID NO: 203 | Light chain constant | RTVAAPSVFIFPPSDEQLKSGTASVVCLLNNFYPREAKV |
| | region | QWKVDNALQSGNSQESVTEQDSKDSTYSLSSTLTLSKA |
| | | DYEKHKVYACEVTHQGLSSPVTKSFNRGEC |
| SEQ ID NO: 242 | IL-2 C125A | APTSSSTKKTQLQLEHLLLDLQMILNGINNYKNPKLTRM |
| | | LTFKFYMPKKATELKHLQCLEEELKPLEEVLNLAQSKNF |
| | | HLRPRDLISNINVIVLELKGSETTFMCEYADETATIVEFLN |
| | | RWITFAQSIISTLT |
| SEQ ID NO: 237 | Linker | GGGGSGGGGS |
| SEQ ID NO: 353 | Heavy chain hinge- | DKTHTCPPCPAPELLGGPSVFLFPPKPKDTLMISRTPEVT |
| | Fc region (CH2- | CVVVDVSHEDPEVKFNWYVDGVEVHNAKTKPREEQYA |
| | CH3) knob | STYRVVSVLTVLHQDWLNGKEYKCKVSNKALPAPIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSP |

TABLE 14
Amino acid sequences of exemplary variable regions of anti-BCMA antibodies.

| SEQ ID | | |
|---|---|---|
| NO | Description | Sequence |
| 354 | 83A10 VH | EVQLLESGGGLVQPGGSLRLSCAASGFTFSSYAMSWVRQAPG |
| | | KGLEWVSAISGSGGSTYYADSVKGRFTISRDNSKNTLYLQMN |
| | | SLRAEDTAVYYCAKVLGWFDYWGQGTLVTVSS |
| 355 | 83A10 VL | EIVLTQSPGTLSLSPGERATLSCRASQSVSSSYLAWYQQKPGQ |
| | | APRLLIYGASSRATGIPDRFSGSGSGTDFTLTISRLEPEDFAVY |
| | | YCQQYGYPPDFTFGQGTKVEIK |
| 356 | 17A5 VH | EVQLLESGGGLVQPGGSLRLSCAASGFTFSSYAMSWVRQAPG |
| | | KGLEWVSAISGSGGSTYYADSVKGRFTISRDNSKNTLYLQMN |
| | | SLRAEDTAVYYCAKVAPYFAPFDYWGQGTLVTVSS |
| 357 | 17A5 VL | EIVLTQSPGTLSLSPGERATLSCRASQSVSSSYLAWYQQKPGQ |
| | | APRLLIYGASSRATGIPDRFSGSGSGTDFTLTISRLEPEDFAVY |
| | | YCQQYGNPPLYTFGQGTKVEIK |
| 358 | 13A4 VH | EVQLVQSGAEVKKPGESLKISCKGSGYSFTSYWIGWVRQMPG |
| | | KGLEWMGIIYPGDSDTRYSPSFQGQVTISADKSISTAYLQWSS |
| | | LKASDTAMYYCARNGYLGDYWGQGTLVTVSS |
| 359 | 13A4 VL | DIVMTQSPLSLPVTPGEPASISCRSSQSLLHSNGYNYLDWYLQ |
| | | KPGQSPQLLIYLGSNRASGVPDRFSGSGSGTDFTLKISRVEAED |
| | | VGVYYCMQAMQIPTFGQGTKVEIK |
| 360 | J22.9-xi | QVQLQQSGGGLVQPGGSLKLSCAASGIDFSRYWMSWVRRAP |
| | VH | GKGLEWIGEINPDSSTINYAPSLKDKFIISRDNAKNTLYLQMSK |
| | | VRSEDTALYYCASLYYDYGDAMDYWGQGTSVTVSS |
| 361 | J22.9-xi | DIVMTQSQRFMTTSVGDRVSVTCKASQSVDSNVAWYQQKPR |
| | VL | QSPKALIFSASLRFSGVPARFTGSGSGTDFTLTISNLQSEDLAE |
| | | YFCQQYNNYPLTFGAGTKLELKR |
| 362 | 2A1 VH | EVQLVESGGGLVKPGGSLRLSCAASGFTFGDYALSWFRQAPG |
| | | KGLEWVGVSRSKAYGGTTDYAASVKGRFTISRDDSKSTAYL |
| | | QMNSLKTEDTAVYYCASSGYSSGWTPFDYWGQGTLVTVSS |
| 363 | 2A1 VL | QSVLTQPPSASGTPGQRVTISCSGSSSNIGSNTVNWYQQLPGT |
| | | APKLLIFNYHQRPSGVPDRFSGSKSGSSASLAISGLQSEDEADY |
| | | YCAAWDDSLNGWVFGGGTKLTVLG |

### Part II

Also provided herein are compositions and method for selective activation/expansion of T cells. Particularly, provided herein are compositions and method for selective activation/expansion of iNKT cells expressing TRAV10 and TRBV25. Without wishing to be bound by theory, the multispecific molecules that targets TRAV10 and/or TRBV25 selectively activates/expands iNKT cell expressing TRAV10 and/or TRBV25 over other T cell sub-populations that do not express TRAV10 and/or TRBV25, providing a novel approach to reduce effects of overactivation caused by pan-T cell activation (e.g., cytokine release syndrome).

In some embodiments, the multispecific molecule of the disclosure binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific molecule binds to an cytokine receptor with a K_{D} that is at least 2, 3, 4, 5, 6, 7, 8, 9, or 10 times greater than that for a corresponding multispecific molecule comprising two or more effector moieties. In another embodiment, the multispecific molecule binds to an cytokine receptor with a dissociation constant K_{D} that is about 10 times greater than that for a corresponding the multispecific molecule comprising two or more cytokines.

In embodiments, the cytokine molecule as used herein includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor. The cytokine molecule can be a monomer or a dimer. In embodiments, the cytokine molecule can further include a cytokine receptor dimerizing domain.

As used herein, the term “T cell receptor alpha variable chain” or “TCRαV,” or “TRAV,” refers to an extracellular region of the T cell receptor alpha chain which can comprise a portion of the antigen recognition domain of the T cell receptor. The term TCRαV includes isoforms, mammalian, e.g., human TCRαV, species homologs of human and analogs comprising at least one common epitope with TCRαV. Human TCRαV comprises a gene family comprising subfamilies including, but not limited to: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, or a TCRα V41 subfamily, as well as family members of said subfamilies, and variants thereof (e.g., a structural or functional variant thereof).

As used herein, the term “T cell receptor beta variable chain” or “TCR4V,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, as well as family members of said subfamilies, and variants thereof (e.g., a structural or functional variant thereof).

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR alpha or TCR beta gene undergoes gene arrangement to generate diversity.

The TCR V alpha or TCR V beta repertoire varies between individuals and populations because of, e.g., 7 frequently occurring inactivating polymorphisms in functional gene segments and a large insertion/deletion-related polymorphism encompassing 2 V alpha or V beta gene segments.

TCR alpha V or TCR beta V families and subfamilies are known in the art, e.g., as described in Yassai et al., (2009) Immunogenetics 61(7)pp: 493-502; Wei S, and Concannon P. (1994) Human Immunology 41(3) pp: 201-206. Exemplary amino acid sequences for TCRαV subfamily members or TCRβV subfamily members can be found on the ImMunoGeneTics Information System website: http://www.imgt.org/, or in a similar resource.

The terms TCRAV, TCRVA, TRAV, TCRαV, TCRVα or TRαV are used interchangeably herein and refer to a TCR alpha V chain as described herein. The terms TCRBV, TCRVB, TRBV, TCRβV, TCRVβ or TRβV are used interchangeably herein and refer to a TCR beta V chain as described herein.

### Invariant Natural Killer T(iNKT) Cells

Invariant natural killer T (iNKT) cells, also known as type I or classical NKT cells, are a distinct population of T cells that express an invariant αβ T-cell receptor (TCR) and a number of cell surface molecules in common with natural killer (NK) cells, iNKT cells are known for their ability to respond rapidly to danger signals and pro-inflammatory cytokines. Once activated, iNKT cells engage in effector functions, e.g., NK transactivation, T cell activation and differentiation, B cell activation, dendritic cell activation and cross-presentation activity, and macrophage activation.

iNKT cells express a restricted TCR repertoire that is composed of an invariant TCRα chain paired with a TCRβ chain with limited VP usage, e.g., TRAV10-TRAJ18 paired with TRBV25 in humans. Unlike conventional T cells, which mostly recognize peptide antigens presented by MHC molecules, iNKT cells recognize glycolipid antigens presented by the non-polymorphic MHC class I-like molecule, CD1d, iNKT cells are frequently characterized by the recognition of the prototypical glycolipid, α-galactosylceramide (α-GalCer), a marine-sponge-derived agent, which also potently activates them and has strong anti-tumor activity.

In addition to being activated through their TCRs in response to CD1d-presented lipids, iNKT cells can be activated by additional immune stimulators, e.g., cytokines (sometimes even without TCR engagement). Non-limiting examples of cytokine that can activate or promote activation of iNKT cells include IL-2, IL-12, IL-15, IL-18, IL-23, IL,25, IL-33, IFN-α, IFN-β, or any functional fragment or variant thereof.

iNKT cell can be divided into subpopulations based on unique cell surface maker/transcription factor expression, cytokine-secretion profile, and/or distinct developmental stages (e.g., iNKT1, iNKT2, iNKT17, iNKTreg, iNKT_{FH}, iNKT10, CD4+, CD8+, CD4− CD8− (DN), etc).

### Development/Sub-Populations/Cytokine-Secretion Profile

iNKT cells have double-positive (DP; CD4+CD8+) thymocytes as progenitors, although a small proportion utilize an alternate pathway (see below). Like conventional T cells, iNKT cells develop through four double-negative (DN) stages (DN1-DN4). While a small subset can mature directly after the DN4 stage through an alternative pathway; the majority of DN iNKT cells transition into the four DP stages of development (S0-S3).

After completing the four DN stages, the DP cells go through four additional stages of development, starting with the random rearrangement of their TCRα loci resulting in the invariant α-chains (Table 15). Unlike DP precursors of conventional T cells, which are selected by thymic epithelial cells, iNKT DP precursors are positively selected when their TCR recognizes self-lipid antigens on CD1d-expressing DP thymocytes. With this unique DP-DP interaction, the positively selected thymocytes are imparted with the developmental program of iNKT cells during S0. This process generates secondary signals and co-stimulation through members of the signal lymphocytic-activated molecules (SLAM) family of receptors. The engagements through the TCR and the SLAM receptors result in downstream expression of the Egr2 transcription factor in the iNKT precursors. During S1, this Egr2 transcription factor is then recruited to the promoter site of the Zbtb16 gene, which encodes the master iNKT transcription factor PLZF. Expression of the PLZF transcription factor is required for guiding the effector properties of the developing iNKT cells. In particular. PLZF is responsible for specifying the migration patterns of iNKT cells to peripheral organs, as well as for the ability to produce cytokines when iNKT cells are stimulated.

iNKT cells migrate out of the thymus at S2 or S3. The iNKT2 and iNKT17 subsets are S2 iNKT-migrated cells, whereas the NKT1 subset develops through S3. These three subsets have lineage-specific transcription factors and produce different combinations of cytokines (Table 16). NKT1 cells express T-box 21 transcription factor and can release interferon gamma (IFNγ). NKT2 cells have GATA binding protein 3 (GATA3) expression and release interleukin (IL)-4 cytokine at steady state. NKT17 cells express the retinoic acid receptor-related orphan nuclear receptor gamma (RORγt) transcription factor and IL-17 cytokine.

The alternative pathway of a small subset of iNKT precursors includes the omission of development through the DP stages. In this alternative development process, precursors from the DN4 stage contain a population that expresses Vαl4Jα18 mRNA, which allows for production of mature DN iNKT cells. Thus, based on CD4 and CD8 expression, mature iNKT cells can be divided into three functionally distinct subsets: CD4⁺ CD8⁻, CD4⁻ CD8⁻, and CD4⁻ CD8⁺.

TABLE 15
iNKT development

| Developmental | | | | |
|---|---|---|---|---|
| stage | Stage 0 | Stage 1 | Stage 2 | Stage 3 |
| Cell-surface | CD4+ | CD4+ | CD4+ | CD4+/− |
| phenotype | CD8+/− | CD8+/− | CD8+/− | CD8+/− |
| | CD24+ | CD24− | CD24− | NK1.1+ |
| | CD69+ | CD69− | CD69− | CD69+ |
| | CCR7+ | CD44low | CD44high | CD44high |
| | CD161(H) | CD161(H) | CD161(H) | CD161(H) |
| | NK1.1 | NK1.1 | NK1.1 | NK1.1 |
| | (M) | (M) | (M) | (M) |
| | | | | CD122+ |
| | | | | CD27+ |

Abbreviations:
H, human;
M, mouse

TABLE 16
Key cytokines secreted by iNKT cells

| iNKT cell subsets | Cytokine released |
|---|---|
| NKT1 | TNFα, IFNγ, IL-2, IL-4, IL-13 |
| NKT2 | IL-4, IL-13, IL-17RB |
| NKT17 | IL-13, IL-17A |

Additionally, iNKT10 cells represent a unique subset of iNKT cells that are PLZF negative but express Nfi13 (E4BP4) and secrete IL-10 to create an immunoregulatory environment, iNKT_{FH} (follicular helper) express Bcl-6 and produce IL-21 to provide help to B cells.

Methods for detecting a cytokine secreted by NKT cell, e.g., Enzyme-linked immunosorbent assay (ELISA), are well-known to a person of ordinary skill in the art.

Identification and isolation of iNKT

Methods for identification and isolation of iNKT cells are known to a person of ordinary skill in the art. For non-limiting examples, human iNKT cells may be identified according to one or more markers listed in Table 17. For non-limiting examples, human iNKT cells may be isolated through flow cytometry from peripheral blood mononuclear cells (PBMCs), e.g., PBMCs may be sorted with antibodies for TCR Vα24Jα18, CD3, CD4, CD8, and CD161 to gate and isolate iNKT cell populations.

TABLE 17
human iNKT cell makers

| | | Marker |
|---|---|---|
| Species | Marker | type |
| Human | CD3 | Surface |
| | CD4 | Surface |
| | CD8 | Surface |
| | CD56 | Surface |
| | TNFα | Intracellular |
| | IFNγ | Intracellular |
| | Vα24Jα18 TCR α-chain (e.g., using a | Surface |
| | fluorescent conjugate of antibody clone | |
| | 6B11) | |

### Compositions of the Present Technology

In one aspect, provided herein is a composition comprising a multispecific molecule, wherein the multispecific molecule comprises: (a) a first domain that binds to a first target molecule, wherein the first target molecule is T cell receptor beta variable 25 (TRBV25) or T cell receptor alpha variable 10 (TRAV10), and (b) a second domain that binds to a second target molecule, wherein the second domain comprises (i) a cytokine or a functional fragment or variant thereof, or (ii) an antigen binding domain.

In some embodiments, the TRAV10 is a TRAV10 polypeptide comprising at least 60%, 65%, 70%, 75%, 80%, 85%, 90%, 95%, 99%, 99.5%, 99.9%, or 100% sequence identity to the sequence of SEQ ID NO: 441.

UniProt_A0A0B4J240_TVA10_HUMAN
(SEQ ID NO: 441)
MKKHLTTFLVILWLYFYRGNGKNQVEQSPQSLIILEGKNCTLQCNYTVS
PFSNLRWYKQDTGRGPVSLTIMTFSENTKSNGRYTATLDADTKQSSLHI
TASQLSDSASYICVVS

In some embodiments, the TRBV25 is TRBV-25-1. In some embodiments, the TRBV25 is TRBV25-1*01. In some embodiments, the TRBV25 is a TRBV10 polypeptide comprising at least 60%, 65%, 70%, 75%, 80%, 85%, 90%, 95%, 99%, 99.5%, 99.9%, or 100% sequence identity to the sequence of SEQ ID NO: 442.

UniProt_A0A075B6N4_TVBY1_HUMAN
(SEQ ID NO: 442)
MTIRLLCYVGFYFLGAGLMEADIYQTPRYLVIGTGKKITLECSQTMGHD
KMYWYQQDPGMELHLIHYSYGVNSTEKGDLSSESTVSRIRTEHFPLTLE
SARPSHTSQYLCASSE

In some embodiments, any domain that binds to TRAV10 as disclosed herein may bind to any epitope of a TRAV10 polypeptide, and any domain that binds to TRBV25 as disclosed herein may bind to any epitope of a TRBV25 polypeptide.

In some embodiments, the composition further comprises an invariant Natural Killer T (iNKT) cell. In some embodiments, the multispecific molecule is bound to the iNKT cell. In some embodiments, the iNKT cell is CD4⁺, CD8⁺ or CD4⁻/CD8⁻. In some embodiments, the iNKT cell is CD4⁺. In some embodiments, the iNKT cell is CD8⁺. In some embodiments, the iNKT cell is CD4⁻/CD8⁻. In some embodiments, the iNKT cell is iNKT1, iNKT2, iNKT7, iNKT10, iNKT_{FH}, or any combination thereof. In some embodiments, the iNKT cell is iNKTreg. In some embodiments, the iNKT cell is iNKT1. In some embodiments, the iNKT cell is iNKT2. In some embodiments, the iNKT cell is iNKT17. In some embodiments, the iNKT cell is iNKT10. In some embodiments, the iNKT cell is iNKT_{FH}. In some embodiments, the iNKT cell is iNKTreg.

In some embodiments, the multispecific molecule is an agonist of TRBV25 or TRAV10. In some embodiments, the multispecific molecule is an antagonist of TRBV25 or TRAV10.

In some embodiments, the second domain comprises a cytokine. In some embodiments, the cytokine comprises IL-2, IL-12, IL-15, IL-18, IL-23, IL-25, IL-33, IFN-α, IFN-β, or any functional fragment or variant thereof. In some embodiments, the multispecific molecule further comprises a third domain comprising an antigen binding domain. In some embodiments, the antigen binding domain of the third domain binds to a tumor associated antigen (TAA). In some embodiments, the TAA is a tumor antigen, a stromal antigen, or a hematological antigen. In some embodiments, the TAA is selected from the group consisting of BCMA. CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-BI, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, αactinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the cytokine of the second domain comprises IL-2 or a functional fragment or variant thereof. In some embodiments, the IL-2 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 443.

UniProt_P60568_IL2_HUMAN
(SEQ ID NO: 443)
MYRMQLLSCIALSLALVTNSAPTSSSTKKTQLQLEHLLLDLQMILNGIN
NYKNPKLTRMLTFKFYMPKKATELKHLQCLEEELKPLEEVLNLAQSKNF
HLRPRDLISNINVIVLELKGSETTFMCEYADETATIVEFLNRWITFCQS
IISTLT

In some embodiments, the IL-2 or the functional fragment or variant thereof comprises a sequence of

(SEQ ID NO: 444)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFAQSIISTLT.

In some embodiments, the IL-2 or the functional fragment or variant thereof comprises a sequence of

(SEQ ID NO: 445)
APASSSTKKTQLQLEHLLLDLQMILNGINNYKNPKLTRMLTAKFAMPKK
ATELKHLQCLEEELKPLEEVLNGAQSKNFHLRPRDLISNINVIVLELKG
SETTFMCEYADETATIVEFLNRWITFAQSIISTLT

In some embodiments, the cytokine of the second domain comprises IL-12 or a functional fragment or variant thereof. In some embodiments, the IL-12 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 446.

UniProt_P29459_IL12A_HUMAN
(SEQ ID NO: 446)
MCPARSLLLVATLVLLDHLSLARNLPVATPDPGMFPCLHHSQNLLRAVS
NMLQKARQTLEFYPCTSEEID
HEDITKDKTSTVEACLPLELTKNESCLNSRETSFITNGSCLASRKTSFM
MALCLSSIYEDLKMYQVEFKTMNAKLLMDPKRQIFLDQNMLAVIDELMQ
ALNENSETVPQKSSLEEPDFYK
TKIKLCILLHAFRIRAVTIDRVMSYLNAS

In some embodiments, the IL-12 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 447.

UniProt_P29460_IL12B_HUMAN
(SEQ ID NO: 447)
MCHQQLVISWFSLVFLASPLVAIWELKKDVYVVELDWYPDAPGEMVVLTCDTPEEDGI
TWTLDQSSEVLGSGKTLTIQVKEFGDAGQYTCHKGGEVLSHSLLLLHKKEDGIWSTDIL
KDQKEPKNKTFLR CEAKNYSGRF TCWWLTTIST DLTFSVKSSR GSSDPQGVTC
GAATLSAERVRGDNKEYEYS
VECQEDSACPAAEESLPIEVMVDAVHKLKYENYTSSFFIRDIIKPDPPKNLQLKPLKNSRQ
VEVSWEYPDTWSTPHSYFSLTFCVQVQGKSKREKKDRVFTDKTSATVICRKNASISVRA
QDRYYSSSWSEWASVPCS

In some embodiments, the IL-12 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 448.

(SEQ ID NO: 448)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCSGGGGSGGGGSGGGGSRNLPVATPDPGMFPCLHHSQNL
LRAVSNMLQKARQTLEFYPCTSEEIDHEDITKDKTSTVEACLPLELTKN
ESCLNSRETSFITNGSCLASRKTSFMMALCLSSIYEDLKMYQVEFKTMN
AKLLMDPKRQIFLDQNMLAVIDELMQALNFNSETVPQKSSLEEPDFYKT
KIKLCILLHAFRIRAVTIDRVMSYLNAS

In some embodiments, the cytokine of the second domain comprises IL-15 or a functional fragment or variant thereof.

In some embodiments, the IL-15 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 449.

UniProt_P40933_IL15_HUMAN
(SEQ ID NO: 449)
MRISKPHLRSISIQCYLCLLLNSHFLTEAGIHVFILGCFSAGLPKTEANWVNVISDLKKIED
LIQSMHIDATLYTESDVHPSCKVTAMKCFLLELQVISLESGDASIHDTVENLIILANNSLSS
NGNVTESGCKECEELE EKNIKEFLQS FVHIVQMFINTS

In some embodiments, the IL-15 or the functional fragment or variant thereof comprises a sequence having at least 60%, 65%, 70%, 75%, 80%, 85%, 90%, 95%, 99%, 99.5%, 99.9%, or 100% sequence identity to the sequence of

(SEQ ID NO: 410)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS.

In some embodiments, the IL-15 or the functional fragment or variant thereof comprises a sequence having at least 60%, 65%, 70%, 75%, 80%, 85%, 90%, 95%, 99%, 99.5%, 99.9%, or 100% sequence identity to the sequence of

(SEQ ID NO: 411)
NWVNVISDLKKIEDLIQSMHIDATLYTESDVHPSCKVTAMKCFLLELQV
ISLASGDASIHDTVENLIILANNSLSSNGAVTESGCKECEELEEKNIKE
FLQSFVHIVQMFINTS.

In some embodiments, the cytokine of the second domain comprises IL-18 or a functional fragment or variant thereof. In some embodiments, the IL-18 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO:412.

UniProt_Q14116_IL18_HUMAN
(SEQ ID NO: 412)
MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFKEMNPPDNIKDTKSDIIFFQRSVPGHDN
KMQFESSSYEGYFLACEKERDLFKLILKKEDELGDRSIMFTVQNED

In some embodiments, the IL-18 or the functional fragment or variant thereof comprises a sequence having at least 60%, 65%, 70%, 75%, 80%, 85%, 90%, 95%, 99%, 99.5%, 99.9%, or 100% sequence identity to the sequence of

(SEQ ID NO: 413)
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED.

In some embodiments, the cytokine of the second domain comprises IL-23 or a functional fragment or variant thereof. In some embodiments, the IL-23 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 407. In some embodiments, the IL-23 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 414.

UniProt_Q9NPF7_IL23A_HUMAN
(SEQ ID NO: 414)
MLGSRAVMLLLLLPWTAQGRAVPGGSSPAWTQCQQLSQKLCTLAWSAHP
LVGHMDLREEGDEETTNDVPHIQCGDGCDPQGLRDNSQFCLQRIHQGLI
FYEKLLGSDIFTGEPSLLPDSPVGQLHASLLGLSQLLQPEGHHWETQQI
PSLSPSQPWQRLLLRFKILRSLQAFVAVAARVFAHGAATLSP

In some embodiments, the cytokine of the second domain comprises IL-25 or a functional fragment or variant thereof. In some embodiments, the IL-25 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 415.

UniProt_Q9H293_IL25_HUMAN
(SEQ ID NO: 415)
MRERPRLGEDSSLISLFLQVVAFLAMVMGTHTYSHWPSCCPSKGQDTSE
ELLRWSTVPVPPLEPARPNRHPESCRASEDGPLNSRAISPWRYELDRDL
NRLPQDLYHARCLCPHCVSLQTGSHMDPRGNSELLYHNQTVFYRRPCHG
EKGTHKGYCLERRLYRVSLACVCVRPRVMG

In some embodiments, the cytokine of the second domain comprises IL-33 or a functional fragment or variant thereof. In some embodiments, the IL-33 or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 416.

UniProt_O95760_IL33_HUMAN
(SEQ ID NO: 416)
MKPKMKYSTNKISTAKWKNTASKALCFKLGKSQQKAKEVCPMYFMKLRS
GLMIKKEACYFRRETTKRPSLKTGRKHKRHLVLAACQQQSTVECFAFGI
SGVQKYTRALHDSSITGISPITEYLASLSTYNDQSITFALEDESYEIYV
EDLKKDEKKDKVLLSYYESQHPSNESGDGVDGKMLMVTLSPTKDFWLHA
NNKEHSVELHKCEKPLPDQAFFVLHNMHSNCVSFECKTDPGVFIGVKDN
HLALIKVDSSENLCTENILFKLSET

In some embodiments, the cytokine of the second domain comprises IFN-α or a functional fragment or variant thereof. In some embodiments, the IFN-α or the functional fragment or variant thereof comprises a sequence having at least 60%, 65%, 70%, 75%, 80%, 85%, 90%, 95%, 99%, 99.5%. 99.9%, or 100% sequence identity to the sequence of any one of SEQ ID NOs: 417-426.

UniProt_P01562_IFNA1_HUMAN
(SEQ ID NO: 417)
MASPFALLMVLVVLSCKSSCSLGCDLPETHSLDNRRTLMLLAQMSRISP
SSCLMDRHDFGFPQEEFDGNQFQKAPAISVLHELIQQIFNLFTTKDSSA
AWDEDLLDKFCTELYQQLNDLEACVMQEERVGETPLMNADSILAVKKYF
RRITLYLTEKKYSPCAWEVVRAEIMRSLSLSTNLQERLRRKE
UniProt_P01563_IFNA2_HUMAN
(SEQ ID NO: 418)
MALTFALLVALLVLSCKSSCSVGCDLPQTHSLGSRRTLMLLAQMRRISL
FSCLKDRHDFGFPQEEFGNQFQKAETIPVLHEMIQQIFNLFSTKDSSAA
WDETLLDKFYTELYQQLNDLEACVIQGVGVTETPLMKEDSILAVRKYFQ
RITLYLKEKKYSPCAWEVVRAEIMRSFSLSTNLQESLRSKE
UniProt_P05014_IFNA4_HUMAN
(SEQ ID NO: 419)
MALSFSLLMAVLVLSYKSICSLGCDLPQTHSLGNRRALILLAQMGRISH
FSCLKDRHDFGFPEEEFDGHQFQKAQAISVLHEMIQQTFNLFSTEDSSA
AWEQSLLEKFSTELYQQLNDLEACVIQEVGVEETPLMNEDSILAVRKYF
QRITLYLTEKKYSPCAWEVVRAEIMRSLSFSTNLQKRLRRKD
UniProt_P01569_IFNA5_HUMAN
(SEQ ID NO: 420)
MALPFVLLMALVVLNCKSICSLGCDLPQTHSLSNRRTLMIMAQMGRISP
FSCLKDRHDFGFPQEEFDGNQFQKAQAISVLHEMIQQTFNLFSTKDSSA
TWDETLLDKFYTELYQQLNDLEACMMQEVGVEDTPLMNVDSI
LTVRKYFQRITLYLTEKKYSPCAWEVVRAEIMRSFSLSANLQERLRRKE
UniProt_P05013_IFNA6_HUMAN
(SEQ ID NO: 421)
MALPFALLMALVVLSCKSSCSLDCDLPQTHSLGHRRTMMLLAQMRRISL
FSCLKDRHDFRFPQEEFDGNQFQKAEAISVLHEVIQQTFNLFSTKDSSV
AWDERLLDKLYTELYQQLNDLEACVMQEVWVGGTPLMNEDSILAVRKYF
QRITLYLTEKKYSPCAWEVVRAEIMRSFSSSRNLQERLRRKE
UniProt_P01567_IFNA7_HUMAN
(SEQ ID NO: 422)
MARSFSLLMVVLVLSYKSICSLGCDLPQTHSLRNRRALILLAQMGRISP
FSCLKDRHEFRFPEEEFDGHQFQKTQAISVLHEMIQQTFNLFSTEDSSA
AWEQSLLEKFSTELYQQLNDLEACVIQEVGVEETPLMNEDFILAVRKYF
QRITLYLMEKKYSPCAWEVVRAEIMRSFSFSTNLKKGLRRKD
UniProt_P32881_IFNA8_HUMAN
(SEQ ID NO: 423)
MALTFYLLVALVVLSYKSFSSLGCDLPQTHSLGNRRALILLAQMRRISP
FSCLKDRHDFEFPQEEFDDKQFQKAQAISVLHEMIQQTFNLFSTKDSSA
ALDETLLDEFYIELDQQLNDLESCVMQEVGVIESPLMYEDSILAVRKYF
QRITLYLTEKKYSSCAWEVVRAEIMRSFSLSINLQKRLKSKE
UniProt_P01566_IFN10_HUMAN
(SEQ ID NO: 424)
MALSFSLLMAVLVLSYKSICSLGCDLPQTHSLGNRRALILLGQMGRISP
FSCLKDRHDFRIPQEEFDGNQFQKAQAISVLHEMIQQTFNLFSTEDSSA
AWEQSLLEKFSTELYQQLNDLEACVIQEVGVEETPLMNEDSILAVRKYF
QRITLYLIERKYSPCAWEVVRAEIMRSLSFSTNLQKRLRRKD
UniProt_P01570_IFN14_HUMAN
(SEQ ID NO: 425)
MALPFALMMALVVLSCKSSCSLGCNLSQTHSLNNRRTLMLMAQMRRISP
FSCLKDRHDFEFPQEEFDGNQFQKAQAISVLHEMMQQTFNLFSTKNSSA
AWDETLLEKFYIELFQQMNDLEACVIQEVGVEETPLMNEDSILAVKKYF
QRITLYLMEKKYSPCAWEVVRAEIMRSLSFSTNLQKRLRRKD
UniProt_P01568_IFN21_HUMAN
(SEQ ID NO: 426)
MALSFSLLMAVLVLSYKSICSLGCDLPQTHSLGNRRALILLAQMGRISP
FSCLKDRHDFGFPQEEFDGNQFQKAQAISVLHEMIQQTFNLFSTKDSSA
TWEQSLLEKFSTELNQQLNDLEACVIQEVGVEETPLMNVDSILAVKKYF
QRITLYLTEKKYSPCAWEVVRAEIMRSFSLSKIFQERLRRKE

In some embodiments, the cytokine of the second domain comprises IFN-β or a functional fragment or variant thereof. In some embodiments, the IFN-β or the functional fragment or variant thereof comprises a sequence having at least 60%, 65%, 70%, 75%, 80%, 85%, 90%, 95%, 99%, 99.5%, 99.9%, or 100% sequence identity to the sequence of SEQ ID NO: 427.

UniProt_P01574_IFNB_HUMAN
(SEQ ID NO: 427)
MTNKCLLQIALLLCFSTTALSMSYNLLGFLQRSSNFQCQKLLWQLNGRL
EYCLKDRMNFDIPEEIKQLQQFQKEDAALTIYEMLQNIFAIFRQDSSST
GWNETIVENLLANVYHQINHLKTVLEEKLEKE
DFTRGKLMSSLHLKRYYGRILHYLKAKEYSHCAWTIVRVEILRNFYFIN
RLTGYLRN

In some embodiments, the second domain comprises an antigen binding domain.

In some embodiments, the antigen binding domain of the second domain binds to TRBV25 or TRAV10. In some embodiments, the first domain binds to TRBV25 and the antigen binding domain of the second domain binds to TRBV25. In some embodiments, the first domain binds to TRAV10 and the antigen binding domain of the second domain binds to TRAV10. In some embodiments, the first domain binds to TRAV25 and the antigen binding domain of the second domain binds to TRAV10, or the first domain binds to TRAV10 and the antigen binding domain of the second domain binds to TRBV25.

In some embodiments, the antigen binding domain of the second domain binds to a tumor associated antigen (TAA). In some embodiments, the TAA is a tumor antigen, a stromal antigen, or a hematological antigen. In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R. β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W. In some embodiments, the multispecific molecule further comprises a third domain comprising a cytokine (e.g., IL-2, IL-12, IL-15, IL-18, IL-23, IL-25, IL-33, IFN-α, IFN-β, or any functional fragment or variant thereof).

In some embodiments, the multispecific molecule comprises a single TRBV25- or TRAV10-binding moiety.

In some embodiments, the multispecific molecule comprises a first polypeptide and a second polypeptide, wherein the first polypeptide and the second polypeptide are non-contiguous, wherein
(i) the first polypeptide comprises a first portion of a dimerization module linked to
   (A) the first domain that binds to TRBV25 or TRAV10, wherein the first domain that binds to TRBV25 or TRAV10 comprises a single chain variable fragment (scFv), or a single domain antibody (sdAb), or
   (B) a first portion of the first domain that binds to TRBV25 or TRAV10, wherein the first portion of the first domain that binds to TRBV25 or TRAV10 comprises a heavy chain variable domain (VH),
   wherein when the first polypeptide comprises the first portion of the first domain that binds to TRBV25 or TRAV10, the multispecific molecule further comprises a third polypeptide comprising a second portion of the first domain that binds to TRBV25 or TRAV10, wherein the second portion of the first domain that binds to TRBV25 or TRAV10 comprises a light chain variable domain (VL), wherein the third polypeptide is non-contiguous with the first polypeptide and the second polypeptide; and
(ii) the second polypeptide comprises a second portion of the dimerization module; wherein
   (a) the cytokine or functional fragment or variant thereof is covalently linked to the first polypeptide, the second polypeptide, or the third polypeptide, or
   (b) the antigen binding domain is covalently linked to the first polypeptide, the second polypeptide, or the third polypeptide.

In some embodiments, the first polypeptide comprises the first portion of the first domain that binds to TRBV25 or TRAV10, and wherein
(i) the first portion of the first domain that binds to TRBV25 or TRAV10 is linked to the N-terminus of the first polypeptide comprising the first portion of the dimerization module; and
(ii) the second polypeptide is linked to the C-terminus of a cytokine or a functional fragment or variant thereof (e.g., IL-2, IL-12, IL-15, IL-18, IL-23, IL-25, IL-33, IFN-α, IFN-β, or any functional fragment or variant thereof).

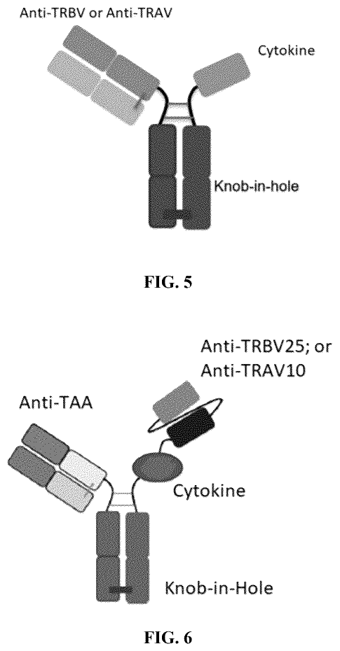

In some embodiments, the multispecific molecule is as shown in FIG. 5.

In some embodiments, the multispecific molecule comprises a first polypeptide and a second polypeptide, wherein the first polypeptide and the second polypeptide are non-contiguous,
wherein the first polypeptide comprises each of the following linked together:
   (i) a first portion of a dimerization module; and
   (ii) (A) a third domain comprising an antigen binding domain, wherein the third domain comprises a single chain variable fragment (scFv), or a single domain antibody (sdAb), or
      (B) a first portion of the third domain, wherein the first portion of the third domain comprises a heavy chain variable domain (VH), wherein when the first polypeptide comprises the first portion of the third domain, the multispecific molecule further comprises a third polypeptide comprising a second portion of the third domain, wherein the second portion of the third domain comprises a light chain variable domain (VL), wherein the third polypeptide is non-contiguous with the first polypeptide and the second polypeptide; and
wherein the second polypeptide comprises each of the following linked together:
   (i) a second portion of the dimerization module;
   (ii) the second domain, wherein the second domain comprises a cytokine (e.g., IL-2, IL-12, IL-15, IL-18, IL-23, IL-25, IL-33, IFN-α, IFN-β, or any functional fragment or variant thereof);
   (iii) (A) the first domain, wherein the first domain comprises a single chain variable fragment (scFv), or a single domain antibody (sdAb), or
      (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the first polypeptide comprises the first portion of the third domain comprising an antigen binding domain, and the second polypeptide comprises the first domain that binds to TRBV25 or TRAV10, wherein the first portion of the third domain is linked to the N-terminus of the first polypeptide comprising the first portion of the dimerization module. In some embodiments, the second polypeptide comprises from N-terminus to C-terminus: the first domain that binds to TRBV25 or TRAV10, the second domain comprising a cytokine (e.g., IL-2, IL-12, IL-15, IL-18, IL-23, IL-25, IL-33, IFN-α, IFN-β, or any functional fragment or variant thereof), and second portion of the dimerization module. In some embodiments, the first domain that binds to TRBV25 or TRAV10 comprises a single chain variable fragment (scFv). In some embodiments, the multispecific molecule is as shown in FIG. 6.

In some embodiments, the multispecific molecule comprises a first polypeptide and a second polypeptide, wherein the first polypeptide and the second polypeptide are non-contiguous, and
wherein the first polypeptide comprises each of the following linked together:
   (i) a first portion of a dimerization module:
   (ii) (A) the second domain comprising an antigen binding domain, wherein the second domain comprises a single chain variable fragment (scFv), or a single domain antibody (sdAb), or
      (B) a first portion of the second domain, wherein the first portion of the second domain comprises a heavy chain variable domain (VH), wherein when the first polypeptide comprises the first portion of the second domain, the multispecific molecule further comprises a third polypeptide comprising a second portion of the second domain, wherein the second portion of the second domain comprises a light chain variable domain (VL), wherein the third polypeptide is non-contiguous with the first polypeptide and the second polypeptide; and
wherein the second polypeptide comprises each of the following linked together:
   (i) a second portion of the dimerization module; and
   (ii) (A) the first domain, wherein the first domain comprises a single chain variable fragment (scFv), or a single domain antibody (sdAb), or
      (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

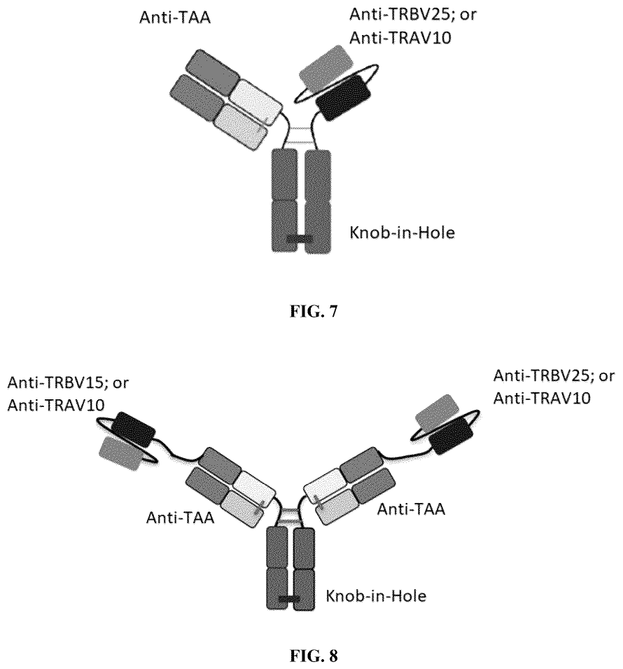

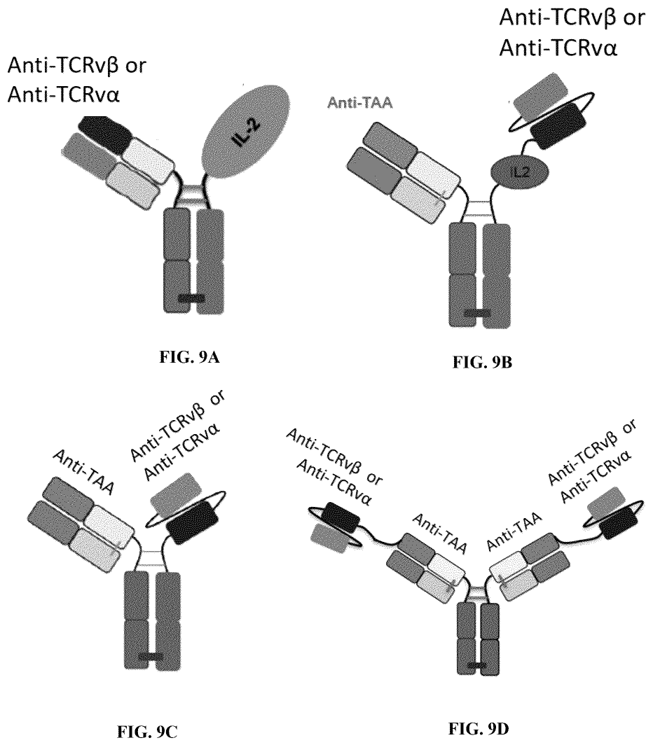

In some embodiments, the first polypeptide comprises the first portion of the second domain comprising an antigen binding domain, and the second polypeptide comprises the first domain that binds to TRBV25 or TRAV10, wherein the first portion of the second domain is linked to the N-terminus of the first polypeptide comprising the first portion of the dimerization module, and the first domain that binds to TRBV25 or TRAV10 is linked to the N-terminus of the second polypeptide comprising the second portion of the dimerization module. In some embodiments, the first domain that binds to TRBV25 or TRAV10 comprises a single chain variable fragment (scFv). In some embodiments, the multispecific molecule is as shown in FIG. 7.

In some embodiments, the multispecific molecule comprises a first polypeptide and a second polypeptide, wherein the first polypeptide and the second polypeptide are non-contiguous, and
wherein the first polypeptide comprises each of the following linked together:
   (i) a first portion of a dimerization module:
   (ii) (A) the first domain, wherein the first domain comprises a single chain variable fragment (scFv), or a single domain antibody (sdAb), or
      (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and
   (iii) (A) the second domain, wherein the second domain comprises the cytokine or a functional fragment or variant thereof, or a single chain variable fragment (scFv), or a single domain antibody (sdAb), or
      (B) a first portion of the second domain, wherein the second domain comprises the antigen binding domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide;
wherein the second polypeptide comprises each of the following linked together:
   (i) a second portion of the dimerization module;
   (ii) (A) an additional first domain, wherein the additional first domain comprises a single chain variable fragment (scFv), or a single domain antibody (sdAb), or
      (B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and
   (iii) (A) an additional second domain, wherein the second domain comprises the cytokine or a functional fragment or variant thereof, or a single chain variable fragment (scFv), or a single domain antibody (sdAb), or
      (B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide, and/or the fifth polypeptide, the sixth polypeptide is non-contiguous with the third polypeptide, the fourth polypeptide, and the fifth polypeptide.

In some embodiments, the first polypeptide comprises the first domain that binds to TRBV25 or TRAV10 and the first portion of the second domain. In some embodiments, the first domain that binds to TRBV25 or TRAV10 is linked to the N-terminus of the first portion of the second domain. In some embodiments, the first portion of the second domain is linked to the N-terminus of the first portion of the dimerization module. In some embodiments, the first domain that binds to TRBV25 or TRAV10 comprises a scFv.

In some embodiments, the second polypeptide comprises the additional first domain that binds to TRBV25 or TRAV10 and the first portion of the additional second domain. In some embodiments, the additional first domain that binds to TRBV25 or TRAV10 is linked to the N-terminus of the first portion of the additional second domain. In some embodiments, the first portion of the second domain is linked to the N-terminus of the first portion of the dimerization module. In some embodiments, the additional first domain that binds to TRBV25 or TRAV10 comprises a scFv.

In some embodiments, the first domain and the additional first domain bind to different targets (i.e., one binds to TRBV25 and the other binds to TRAV10). In some embodiments, the first domain and the additional first domain bind to the same target (i.e., TRBV25 or TRAV10). In some embodiments, the multispecific molecule is as shown in FIG. 8.

In some embodiments, the second domain and the additional second domain bind to the same antigens (e.g., same TAA as disclosed herein). In some embodiments, the second domain and the additional second domain bind to different antigens (e.g., different TAAs as disclosed herein).

In some embodiments, the first portion of the dimerization module comprises a first immunoglobulin constant region (Fc region) and the second portion of the dimerization module comprises a second Fc region. In some embodiments, the first Fc region, the second Fc region, or a combination thereof is selected from the group consisting of IgG1, IgG2, IgG3, IgG4, IgA1, IgA2, IgJ, IgM, IgD, IgE, and any fragment thereof. In some embodiments, the first Fc region, the second Fc region, or a combination thereof comprises an IgGI constant region comprising one or more amino acid substitutions selected from the group consisting of N297A, K322A, L234A and L235A. In some embodiments, the first Fc region, the second Fc region, or a combination thereof comprises a sequence having at least 75% sequence identity to the sequence of SEQ ID NO: 430, SEQ ID NO: 432, SEQ ID NO: 437, SEQ ID NO: 438, SEQ ID NO: 439, SEQ ID NO: 440, or SEQ ID NO: 441.

In any of the above embodiments, the first domain that binds to a first target molecule, the second domain that binds to a second target molecule, or both comprises a Fab, a F(ab′)2, an Fv, a single chain Fv (scFv), a single domain antibody, a diabody (dAb), a camelid antibody, and a combination thereof. In any of the above embodiments, the first domain that binds to a first target molecule, the second domain that binds to a second target molecule, or both comprises a Fab or an scFv. In any of the above embodiments, the first domain that binds to TRBV25 or TRAV10, a second domain that binds to TRBV25 or TRAV10, or both comprises a scFv.

In any of the above embodiments, the first domain that binds to
(i) TRBV25 comprises a VH comprising complementarity determining regions (VH-CDR1-3) of SEQ ID NOs: 461-463, respectively, and a VL comprising VL-CDR1-3 of SEQ ID NOs: 465-467, respectively; or
(ii) TRAV10 comprises a VH comprising complementarity determining regions (VH-CDR1-3) of SEQ ID NOs: 453-455, respectively, and a VL comprising VL-CDR1-3 of SEQ ID NOs: 457-459, respectively.

In any of the above embodiments, the first domain that binds to
(i) TRBV25 comprises a VH comprising a sequence having at least 75% sequence identity to the sequence of SEQ ID NO: 460, and a VL comprising a sequence having at least 75% sequence identity to the sequence of SEQ ID NO: 464; or
(ii) TRAV10 comprises a VH comprising a sequence having at least 75% sequence identity to the sequence of SEQ ID NO: 452, and a VL comprising a sequence having at least 75% sequence identity to the sequence of SEQ ID NO: 456.

In some embodiments, the first domain binds to TRBV25. In some embodiments, the composition further comprises a second multispecific molecule, wherein the second multispecific molecule comprises a fourth domain that binds to TRAV10.

In some embodiments, the first domain binds to TRAV10. In some embodiments, the composition further comprises a second multispecific molecule, wherein the second multispecific molecule comprises a fourth domain that binds to TRBV25.

In some embodiments, the second multispecific molecule comprises a fifth domain, wherein the fifth domain comprises (i) a cytokine or a functional fragment or variant thereof (e.g., IL-2, IL-12, IL-15, IL-18, IL-23, IL-25, IL-33, IFN-α, IFN-0, or any functional fragment or variant thereof), or (ii) an antigen binding domain.

In some embodiments, the fifth domain comprises a cytokine (e.g., IL-2, IL-12, IL-15, IL-18, IL-23, IL-25, IL-33, IFN-α, IFN-β, or any functional fragment or variant thereof). In some embodiments, the second multispecific molecule comprises a sixth domain comprising an antigen binding domain. In some embodiments, the antigen binding domain of the sixth domain binds to TRBV25 or TRAV10. In some embodiments, the antigen binding domain of the sixth domain binds to a TAA.

In some embodiments, the fifth domain comprises an antigen binding domain. In some embodiments, the second multispecific molecule comprises a sixth domain comprising a cytokine (e.g., IL-2, IL-12, IL-15, IL-18, IL-23, IL-25, IL-33, IFN-α, IFN-β, or any functional fragment or variant thereof). In some embodiments, the antigen binding domain of the fifth domain binds to TRBV25 or TRAV10. In some embodiments, the antigen binding domain of the fifth domain binds to a TAA.

In another aspect, provided herein is a composition comprising a multispecific molecule, wherein the multispecific molecule comprises
(a) a first polypeptide and a second polypeptide, wherein the first polypeptide and the second polypeptide are non-contiguous, and
(b) a first domain that binds to a first target molecule, wherein the first target molecule is a TRBV or a TRAV, and
(c) a second domain that binds to a second target molecule, wherein the second domain comprises an antigen binding domain,
wherein the first polypeptide comprises each of the following linked together:
   (i) a first portion of a dimerization module;
   (ii) (A) the first domain, wherein the first domain comprises a single chain variable fragment (scFv), or a single domain antibody (sdAb), or
      (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and
   (iii) (A) the second domain, wherein the second domain comprises a single chain variable fragment (scFv), or a single domain antibody (sdAb), or
      (B) a first portion of the second domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide;
wherein the second polypeptide comprises a second portion of the dimerization module.

In some embodiments, the second polypeptide comprises
(A) an additional first domain, wherein the additional first domain comprises a single chain variable fragment (scFv), or a single domain antibody (sdAb), or
(B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide.

In some embodiments, the second polypeptide comprises
(A) an additional second domain, wherein the additional second domain comprises a cytokine or a functional fragment or variant thereof (e.g., IL-2, IL-12, IL-15, IL-18, IL-23, IL-25, IL-33, IFN-α, IFN-0, or any functional fragment or variant thereof), or a single chain variable fragment (scFv), or a single domain antibody (sdAb), or
(B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide and/or the fifth polypeptide, the sixth polypeptide is non-contiguous with the third polypeptide, the fourth polypeptide, and the fifth polypeptide.

In some embodiments, the second polypeptide comprises
(i) (A) an additional first domain, wherein the additional first domain comprises a single chain variable fragment (scFv), or a single domain antibody (sdAb), or
   (B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and/or
(ii) (A) an additional second domain, wherein the additional second domain comprises a cytokine or a functional fragment or variant thereof (e.g., IL-2, IL-12, IL-15, IL-18, IL-23, IL-25, IL-33, IFN-α, IFN-β, or any functional fragment or variant thereof), or a single chain variable fragment (scFv), or a single domain antibody (sdAb), or
   (B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide and/or the fifth polypeptide, the sixth polypeptide is non-contiguous with the third polypeptide, the fourth polypeptide, and the fifth polypeptide.

In some embodiments, the first polypeptide comprises the first domain that binds to TRBV or TRAV and the first portion of the second domain comprising the antigen binding domain. In some embodiments, the first domain that binds to TRBV or TRAV is linked to the N-terminus of the first portion of the second domain comprising the antigen binding domain. In some embodiments, the first portion of the second domain is linked to the N-terminus of the first portion of the dimerization module. In some embodiments, the first domain that binds to TRBV or TRAV comprises a scFv.

In some embodiments, the second polypeptide comprises the additional first domain that binds to TRBV or TRAV and the first portion of the additional second domain comprising the additional antigen binding domain. In some embodiments, the additional first domain that binds to TRBV or TRAV is linked to the N-terminus of the first portion of the additional second domain comprising the additional antigen binding domain. In some embodiments, the first portion of the second domain is linked to the N-terminus of the first portion of the dimerization module. In some embodiments, the additional first domain that binds to TRBV or TRAV comprises a scFv.

In some embodiments, the first domain and the additional first domain bind to the same TRBV or TRAV. In some embodiments, the first domain and the additional first domain bind to the different targets.

In another aspect, provided herein is a composition comprising a multispecific molecule, wherein the multispecific molecule comprises
(a) a first polypeptide and a second polypeptide, wherein the first polypeptide and the second polypeptide are non-contiguous, and
(b) a first domain that binds to a first target molecule, wherein the first target molecule is a TRBV or a TRAV, and
(c) a second domain that binds to a second target molecule, wherein the second domain comprises (i) a cytokine or a functional fragment or variant thereof (e.g., IL-2, IL-12, IL-15, IL-18, IL-23, IL-25, IL-33, IFN-α, IFN-β, or any functional fragment or variant thereof), or (ii) an antigen binding domain;
wherein the first polypeptide comprises each of the following linked together:
   (i) a first portion of a dimerization module:
   (ii) (A) the first domain, wherein the first domain comprises a single chain variable fragment (scFv), or a single domain antibody (sdAb), or
      (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and
   (iii) (A) the second domain, wherein the second domain comprises the cytokine or a functional fragment or variant thereof, or a single chain variable fragment (scFv), or a single domain antibody (sdAb), or
      (B) a first portion of the second domain, wherein the second domain comprises the antigen binding domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide;
wherein the second polypeptide comprises each of the following linked together:
   (i) a second portion of the dimerization module:
   (ii) (A) an additional first domain, wherein the additional first domain comprises a single chain variable fragment (scFv), or a single domain antibody (sdAb), or
      (B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and
   (iii) (A) an additional second domain, wherein the second domain comprises the cytokine or a functional fragment or variant thereof, or a single chain variable fragment (scFv), or a single domain antibody (sdAb), or
      (B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide, and/or the fifth polypeptide, the sixth polypeptide is non-contiguous with the third polypeptide, the fourth polypeptide, and the fifth polypeptide.

In some embodiments, the additional first domain binds the same target molecule as the first domain.

In some embodiments, the additional antigen binding domain of the additional second domain binds the same target molecule as the antigen binding domain of the second domain.

In some embodiments, the cytokine of the additional second domain comprises the same cytokine as the cytokine of the second domain.

In some embodiments, the second domain of the first polypeptide comprises the cytokine or a functional fragment or variant thereof, and the additional second domain of the second polypeptide comprises the cytokine or a functional fragment or variant thereof.

In some embodiments, (Ia) the second domain of the first polypeptide comprises the scFv or the sdAb or (IIa) the first polypeptide comprises the first portion of the second domain, and (Ib) the additional second domain of the second polypeptide comprises the scFv or the sdAb or (IIb) the second polypeptide comprises the first portion of the additional second domain.

In some embodiments, (Ia) the second domain of the first polypeptide comprises the scFv or the sdAb or (IIa) the first polypeptide comprises the first portion of the second domain, and (Ib) the additional second domain of the second polypeptide comprises the cytokine or a functional fragment or variant thereof.

In some embodiments, (Ia) the second domain of the first polypeptide comprises the cytokine or a functional fragment or variant thereof, and (Ib) the additional second domain of the second polypeptide comprises the scFv or the sdAb or (IIb) the second polypeptide comprises the first portion of the additional second domain.

In some embodiments, the first polypeptide comprises the first domain that binds to TRBV or TRAV and the first portion of the second domain comprising the VH. In some embodiments, the first domain that binds to TRBV or TRAV is linked to the N-terminus of the first portion of the second domain comprising the VH. In some embodiments, the first portion of the second domain comprising the VH is linked to the N-terminus of the first portion of the dimerization module. In some embodiments, the first domain that binds to TRBV or TRAV comprises a scFv.

In some embodiments, the second polypeptide comprises the additional first domain and the first portion of the additional second domain. In some embodiments, the additional first domain is linked to the N-terminus of the first portion of the additional second domain. In some embodiments, the first portion of the second domain is linked to the N-terminus of the first portion of the dimerization module. In some embodiments, the additional first domain that binds to TRBV or TRAV comprises a scFv.

Exemplary TRBV include but are not limited to TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBVlO-1, TRBV10-2, TRBVlO-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1, and TRBV30.

In some embodiments, any domain that binds to TRBV as disclosed herein binds to an outward facing epitope of a TRBV protein when it is in a complex with a TCR α chain. In some embodiments, any domain that binds to TRBV as disclosed herein does no bind to an interface of a TCRβV:TCRα complex. In some embodiments, any domain that binds to TRBV as disclosed herein does no bind to a constant region of a TCR β chain.

Exemplary anti-TRBV antibody molecules include but are not limited to those listed in Table 18.

TABLE 18
Exemplary anti-TRBV antibody molecules

| TRBV | TRBV | Reagents monoclonal antibodies | | |
|---|---|---|---|---|
| gene | allele | Clone name and | | |
| name | name | Specificity | Company product | Isotype |
| TRBV2 | TRBV2*01 | IsMMU 546 (TRBV2) | Serotec V BETA 22 | Mouse |
| | TRBV2*02 | | Coulter Vbeta22 | IgG1 |
| | TRBV2*03 | | | |
| TRBV3-1 | TRBV3- | FIN9 (TRBV3-1) | Serotec Vbeta9 | Mouse |
| | 1*01 | AMKB1-2 (TRBV3-1) | Coulter Vbeta9 | IgG2a |
| | TRBV3- | | BD Biosciences Vbeta9 | Mouse |
| | 1*02 | | | IgG1 |
| TRBV4-1 | TRBV4- | ZOE (TRBV4-1, | Serotec V BETA 7 | Mouse |
| | 1*01 | TRBV4-2, TRBV4-3) | Coulter Vbeta7 | IgG2a |
| | TRBV4- | 3G5 (TRBV4-1) | Pierce EndogenV beta 7.1 | Mouse |
| | 1*02 | | | IgG2b |
| TRBV4-2 | TRBV4- | ZOE (TRBV4-1, | Serotec V BETA 7 | Mouse |
| | 2*01 | TRBV4-2, TRBV4-3) | Coulter Vbeta7 | IgG2a |
| | TRBV4- | | | |
| | 2*02 | | | |
| TRBV4-3 | TRBV4- | ZOE (TRBV4-1, | Serotec V BETA 7 | Mouse |
| | 3*01 | TRBV4-2, TRBV4-3) | Coulter Vbeta7 | IgG2a |
| | TRBV4- | | | |
| | 3*02 | | | |
| | TRBV4- | | | |
| | 3*03 | | | |
| | TRBV4- | ZIZOU4 (TRBV4-3) | Coulter Vbeta7.2 | Mouse |
| | 3*04 | | | IgG2a |
| TRBV5-1 | TRBV5- | IMMU157 (TRBV5-1) | Serotec Vbeta5.1 | Mouse |
| | 1*01 | | Coulter Vbeta5.1 | IgG2a |
| | TRBV5- | LC4 (TRBV5-1) | Pierce Endogen V beta | Mouse |
| | 1*02 | | 5(c) | IgG1 |
| | | | BD Biosciences Vbeta5(c) | |
| TRBV5-4 | TRBV5- | | | |
| | 4*01 | | | |
| | TRBV5- | | | |
| | 4*02 | | | |
| | TRBV5- | | | |
| | 4*03 | | | |
| | TRBV5- | | | |
| | 4*04 | | | |
| TRBV5-5 | TRBV5- | 3D11 (TRBV5-5) | Serotec VBETA5.3 | Mouse |
| | 5*01 | 1C1 (TRBV5-5, | Coulter Vbeta5.3 | IgG1 |
| | TRBV5- | TRBV5-6) | Pierce Endogen V beta | Mouse |
| | 5*02 | W112 (TRBV5-5) | 5(a) | IgG1 |
| | | MH3-2 (TRBV5-5, | BD Biosciences Vbeta5(a) | |
| | TRBV5- | TRBV5-6) | Pierce Endogen V beta | Mouse |
| | 5*03 | | 5(b) | IgG1 |
| | | | Serotec V beta 5.2/5.3 | |
| | | | BD Biosciences | |
| | | | Vbeta5(b) | |
| | | | BD Biosciences Vbeta5 | Mouse |
| | | | | IgG2a |
| | | 4H11 (TM27) as | | |
| | | disclosed in U.S. Pat. No. | | |
| | | 5,861,155 | | |
| TRBV5-6 | TRBV5- | 36213 (TRBV5-6) | Serotec Vbeta5.2 | Mouse |
| | 6*01 | 1C1 (TRBV5-5, | BD Biosciences Vbeta5(a) | IgG1 |
| | | TRBV5-6) | BD Biosciences Vbeta5 | Mouse |
| | | MH3-2 (TRBV5-5, | | IgG1 |
| | | TRBV5-6) | | Mouse |
| | | | | IgG2a |
| TRBV5-8 | TRBV5- | | | |
| | 8*01 | | | |
| | TRBV5- | | | |
| | 8*02 | | | |
| TRBV6-1 | TRBV6- | BAM13 (TRBV6-1, | Pierce Endogen V beta 13 | Mouse |
| | 1*01 | TRBV6-5) | BD Biosciences | IgG1 |
| | | | Vbeta13.1, 13.3 | |
| TRBV6-2 | TRBV6- | H132 | Coulter Vbeta13.2 | Mouse |
| | 2*01 | | | IgG1 |
| TRBV6-3 | TRBV6- | | | |
| | 3*01 | | | |
| TRBV6-4 | TRBV6- | | | |
| | 4*01 | | | |
| | TRBV6- | | | |
| | 4*02 | | | |
| TRBV6-5 | TRBV6- | IMMU 222 (TRBV6- | Serotec V BETA 13.1 | Mouse |
| | 5*01 | 5, TRBV6-6 and | Coulter Vbeta13.1 | IgG2b |
| | | TRBV6-9) | Pierce Endogen V beta 13 | Mouse |
| | | BAM13 (TRBV6-1, | BD Biosciences | IgG1 |
| | | TRBV6-5) | Vbeta13.1, 13.3 | |
| TRBV6-6 | TRBV6- | JU-74 (TRBV6-6) | Serotec Vbeta13.6 | Mouse |
| | 6*01 | JU74.3 (TRBV6-6) | Coulter Vbeta13.6 | IgG1 |
| | TRBV6- | IMMU 222 (TRBV6- | | |
| | 6*02 | 5, TRBV6-6 and | | |
| | TRBV6- | TRBV6-9) | | |
| | 6*03 | | | |
| | TRBV6- | | Serotec V BETA 13.1 | Mouse |
| | 6*04 | | Coulter Vbeta13.1 | IgG2b |
| | TRBV6- | | | |
| | 6*05 | | | |
| TRBV6-8 | TRBV6- | | | |
| | 8*01 | | | |
| TRBV6-9 | TRBV6- | IMMU 222 (TRBV6- | Serotec V BETA 13.1 | Mouse |
| | 9*01 | 5, TRBV6-6 and | Coulter Vbeta13.1 | IgG2b |
| | | TRBV6-9) | | |
| TRBV7-2 | TRBV7- | OT145 (TRBV7-2) | Pierce Endogen V beta 6.7 | Mouse |
| | 2*01 | | BD Biosciences Vbeta6.7 | IgG1 |
| | TRBV7- | | | |
| | 2*02 | | | |
| | TRBV7- | | | |
| | 2*03 | | | |
| | TRBV7- | | | |
| | 2*04 | | | |
| TRBV7-3 | TRBV7- | | | |
| | 3*01 | | | |
| | TRBV7- | | | |
| | 3*04 | | | |
| | TRBV7- | | | |
| | 3*05 | | | |
| TRBV7-4 | TRBV7- | | | |
| | 4*01 | | | |
| TRBV7-6 | TRBV7- | | | |
| | 6*01 | | | |
| | TRBV7- | | | |
| | 6*02 | | | |
| TRBV7-7 | TRBV7- | | | |
| | 7*01 | | | |
| | TRBV7- | | | |
| | 7*02 | | | |
| TRBV7-8 | TRBV7- | | | |
| | 8*01 | | | |
| | TRBV7- | | | |
| | 8*02 | | | |
| | TRBV7- | | | |
| | 8*03 | | | |
| TRBV7-9 | TRBV7- | | | |
| | 9*01 | | | |
| | TRBV7- | | | |
| | 9*02 | | | |
| | TRBV7- | | | |
| | 9*03 | | | |
| | TRVB7- | | | |
| | 9*04 | | | |
| | TRBV7- | | | |
| | 9*05 | | | |
| | TRBV7- | | | |
| | 9*06 | | | |
| | TRBV7- | | | |
| | 9*07 | | | |
| TRBV9 | TRBV9*01 | BL37.2 (TRBV9) | Serotec Vbeta1 | Rat |
| | TRBV9*02 | | Coulter Vbeta1 | IgG1 |
| | TRBV9*03 | | | |
| TRBV10-1 | TRBV10- | S511 (TRBV10-1, | Pierce Endogen V beta 12 | Mouse |
| | 1*01 | TRBV10-2, TRBV10- | BD Biosciences Vbeta12 | IgG2b |
| | TRBV10- | 3) | | |
| | 1*02 | | | |
| TRBV10-2 | TRBV10- | | | |
| | 2*01 | | | |
| | TRBV10- | | | |
| | 2*02 | | | |
| TRBV10-3 | TRBV10- | VER2.32.1 (TRBV10- | Serotec Vbeta12 | Mouse |
| | 3*01 | 3) | Coulter Vbeta12 | IgG2a |
| | TRBV10- | S511 (TRBV10-1, | | |
| | 3*02 | TRBV10-2, TRBV10- | | |
| | TRBV10- | 3) | Pierce Endogen V beta 12 | Mouse |
| | 3*03 | | BD Biosciences Vbeta12 | IgG2b |
| | TRBV10- | | | |
| | 3*04 | | | |
| TRBV11-1 | TRBV11- | | | |
| | 1*01 | | | |
| TRBV11-2 | TRBV11- | IG125 (TRBV11-2) | Serotec Vbeta21.3 | Mouse |
| | 2*01 | | Coulter Vbeta21.3 | IgG2a |
| | TRBV11- | | | |
| | 2*02 | | | |
| | TRBV11- | | | |
| | 2*03 | | | |
| TRBV11-3 | TRBV11- | | | |
| | 3*01 | | | |
| | TRBV11- | | | |
| | 3*02 | | | |
| | TRBV11- | | | |
| | 3*03 | | | |
| | TRBV11- | | | |
| | 3*04 | | | |
| TRBV12-3 | TRBV12- | 56C5 (TRBV12-3, | Serotec Vbeta8.1/8.2 | Mouse |
| | 3*01 | TRBV12-4) | Coulter Vbeta8 | IgG2a |
| TRBV12-4 | TRBV12- | 56C5.2 (TRBV12-3, | Pierce Endogen V beta | Mouse |
| | 4*01 | TRBV12-4) | 8(a) | IgG2b |
| | | 16G8 (TRBV12-3, | BD Biosciences Vbeta8 | |
| | TRBV12- | TRBV12-4) | Pierce Endogen V beta | Mouse |
| | 4*02 | MX-6 (TRBV12-3, | 8(b) | IgG2a |
| | | TRBV12-4) | BD Biosciences | Mouse |
| | | JR2 (TRBV12-3, | Vbeta8 | IgG2b |
| | | TRBV12-4, TRBV12- | | |
| | | 5) | | |
| TRBV12-5 | TRBV12- | JR2 (TRBV12-3, | BD Biosciences Vbeta8 | Mouse |
| | 5*01 | TRBV12-4, TRBV12- | | IgG2b |
| | | 5) | | |
| TRBV13 | TRBV13*01 | AF-23 (TRBV13) | Serotec Vbeta23 | Mouse |
| | TRBV13*02 | AF23 (TRBV13) | Coulter Vbeta23 | IgG1 |
| | | AHUT7 (Vbeta23) | BD Biosciences Vbeta23 | |
| TRBV14 | TRBV14*01 | TAMAYA1.2 | Serotec Vbeta16 | Mouse |
| | TRBV14*02 | (TRBV14) | Coulter Vbeta16 | IgG1 |
| TRBV15 | TRBV15*01 | | | |
| | TRBV15*02 | | | |
| | TRBV15*03 | | | |
| TRBV16 | TRBV16*01 | | | |
| | TRBV16*03 | | | |
| TRBV18 | TRBV18*01 | BA62 (TRBV18) | Serotec V BETA 18 | Mouse |
| | | BA62.6 (TRBV18) | Coulter Vbeta18 | IgG1 |
| TRBV19 | TRBV19*01 | C1 (TRBV19) | Pierce Endogen V beta 17 | Mouse |
| | | E17.5F3 (TRBV19) | BD Biosciences Vbeta17 | IgG1 |
| | TRBV19*02 | E17.5F3.15.13 | Serotec Vbeta17 | Mouse |
| | TRBV19*03 | (TRBV19) | Coulter Vbeta17 | IgG1 |
| TRBV20-1 | TRBV20- | MPB2D5 (TRBV20-1) | Serotec VBETA2 | Mouse |
| | 1*01 | | Coulter Vbeta2 | IgG1 |
| | TRBV20- | | | |
| | 1*02 | | | |
| | TRBV20- | | | |
| | 1*03 | | | |
| | TRBV20- | | | |
| | 1*04 | | | |
| | TRBV20- | | | |
| | 1*05 | | | |
| | TRBV20- | | | |
| | 1*06 | | | |
| | TRBV20- | | | |
| | 1*07 | | | |
| TRBV24-1 | TRBV24- | | | |
| | 1*01 | | | |
| TRBV25-1 | TRBV25- | C21 (TRBV25-1) | Serotec V BETA 11 | Mouse |
| | 1*01 | | Coulter Vbeta11 | IgG2a |
| TRBV27 | TRBV27*01 | CAS1.1.3 (TRBV27) | Serotec Vbeta14 | Mouse |
| | | | Coulter Vbeta14 | IgG1 |
| TRBV28 | TRBV28*01 | CH92 (TRBV28) | Serotec Vbeta3 | Mouse |
| | | 8F10 (TRBV28) | Coulter Vbeta3 | IgM |
| | | JOVI-3 (TRBV28) | Pierce Endogen V beta 3.1 | Mouse |
| | | | BD Biosciences Vbeta3 | IgG1 |
| | | | | Mouse |
| | | | | IgG2a |
| TRBV29-1 | TRBV29- | WJF24 | Coulter Vbeta4 | Rat |
| | 1*01 | | | IgM |
| | TRBV29- | | | |
| | 1*02 | | | |
| | TRBV29- | | | |
| | 1*03 | | | |
| TRBV30 | TRBV30*01 | ELL1.4 (TRBV30) | Serotec Vbeta20 | Mouse |
| | TRBV30*02 | | Coulter Vbeta20 | IgG1 |
| | TRBV30*04 | | | |
| | TRBV30*05 | | | |

Exemplary TRAV include but are not limited to TRAV1-1, TRAV1-2, TRAV2, TRAV3, TRAV4, TRAV5, TRAV6, TRAV7, TRAV8-1, TRAV8-2, TRAV8-3, TRAV8-4, TRAV8-6, TRAV9-1, TRAV9-2, TRAV10, TRAV12-1, TRAV12-2, TRAV12-3, TRAV13-1, TRAV13-2, TRAV14/DV4, TRAV16, TRAV17, TRAV18, TRAV19, TRAV20, TRAV21, TRAV22, TRAV23, TRAV23/DV6, TRAV24, TRAV25, TRAV26-1, TRAV26-2, TRAV27, TRAV29/DV5, TRAV30, TRAV34, TRAV35, TRAV36, TRAV38-1 TRAV38-2/DV8, TRAV39, TRAV40, and TRAV41.

In some embodiments, any domain that binds to TRAV as disclosed herein binds to an outward facing epitope of a TRAV protein when it is in a complex with a TCR β chain. In some embodiments, any domain that binds to TRBV as disclosed herein does no bind to an interface of aTRAV:TCRβ complex. In some embodiments, any domain that binds to TRAV as disclosed herein does no bind to a constant region of a TCR α chain.

Exemplary anti-TRAV antibody molecules include but are not limited to those listed in Table 19.

TABLE 19
Exemplary anti-TRAV antibody molecules

| | | Reagents | | |
|---|---|---|---|---|
| | | monoclonal | | |
| RAV | TRAV | antibodies | | |
| gene | allele | Clone name and | Company | |
| name | name | Specificity | product | Isotype |
| TRAV1-1 | 1-1*01, *02 | | | |
| TRAV1-2 | 1-2*01, *02 | | | |
| TRAV2 | 2*01, *02 | | | |
| TRAV3 | 3*01 | | | |
| TRAV4 | 4*01 | | | |
| TRAV5 | 5*01 | | | |
| TRAV6 | 6*01 to *06 | | | |
| TRAV7 | 7*01 | | | |
| TRAV8-1 | 8-1*01, *02 | | | |
| TRAV8-2 | 8-2*01, *02 | | | |
| TRAV8-3 | 8-3*01 to *03 | | | |
| TRAV8-4 | 8-4*01 to *07 | | | |
| TRAV8-6 | 8-6*01, *02 | | | |
| TRAV8-7 | 8-7*01 | | | |
| TRAV9-1 | 9-1*01 | | | |
| TRAV9-2 | 9-2*01 to *04 | | | |
| TRAV10 | 10*01 | C15 | Coulter | Mouse |
| | | (TRAV10) | Valpha24 | IgG1 |
| TRAV12-1 | 12-1*01 | | | |
| | 12-1*02 | | | |
| TRAV12-2 | 12-2*01 | | | |
| | TRAV12-2*02 | | | |
| | TRAV12-2*03 | | | |
| TRAV12-3 | 12-3*01 | | | |
| | 12-3*02 | | | |
| TRAV13-1 | 13-1*01 to | | | |
| | *03 | | | |
| TRAV13-2 | 13-2*01, *02 | | | |
| TRAV14/ | 14/DV4*01 to | | | |
| DV4 | *04 | | | |
| TRAV16 | 16*01 | | | |
| TRAV17 | 17*01 | | | |
| TRAV18 | 18*01 | | | |
| TRAV19 | 19*01 | 6D6.6 | Pierce | Mouse |
| | | (TRAV19) | Endogen V | IgG1 |
| | | | alpha 12.1 | |
| TRAV20 | 20*01 to *04 | | | |
| TRAV21 | 21*01, *02 | | | |
| TRAV22 | 22*01 | | | |
| TRAV23/ | 23/DV6*01 to | | | |
| DV6 | *04 | | | |
| TRAV24 | 24*01, *02 | | | |
| TRAV25 | 25*01 | | | |
| TRAV26-1 | 26-1*01 to | | | |
| | *03 | | | |
| TRAV26-2 | 26-2*01, *02 | | | |
| TRAV27 | 27*01 to *03 | | | |
| TRAV29/ | 29/DV5*01, | | | |
| DV5 | *02 | | | |
| TRAV30 | 30*01 to *04 | | | |
| TRAV34 | 34*01 | | | |
| TRAV35 | 35*01, *02 | | | |
| TRAV36/ | 36/DV7*01 to | | | |
| DV7 | *04 | | | |
| TRAV38-1 | 38-1*01 to | | | |
| | *04 | | | |
| TRAV38-2/ | 38-2/DV8*01 | | | |
| DV8 | | | | |
| TRAV39 | 39*01 | | | |
| TRAV40 | 40*01 | | | |
| TRAV41 | 41*01 | | | |

In another aspect, provided herein is a pharmaceutical composition comprising any of the compositions disclosed herein.

Methods of Activating and/or Expanding iNKT Cells of the Present Technology

In some aspect, provided herein is a method of activating/expanding a population of cells comprising a T cell (e.g., an iNKT cell) comprising contacting the population of cells comprising the T cell with any of the compositions disclosed above.

In one aspect, provided herein is a method of expanding a population of cells comprising an invariant Natural Killer T (iNKT) cell comprising contacting the population of cells comprising the iNKT cell with any of the compositions disclosed above.

In one aspect, provided herein is a method of activating a population of cells comprising an invariant Natural Killer T (iNKT) cell comprising contacting the population of cells comprising the iNKT cell with any of the compositions disclosed above.

In another aspect, provided herein is a method of expanding a population of cells comprising an invariant Natural Killer T (iNKT) cell comprising contacting the population of cells comprising the iNKT cell with a composition comprising a molecule comprising a first domain that binds to a first target molecule, thereby expanding the iNKT cell, wherein the first target molecule is TRBV25 or TRAV10.

In vet another aspect, provided herein is a method of activating a population of cells comprising an invariant Natural Killer T (iNKT) cell comprising contacting a population of cells comprising the iNKT cell with a composition comprising a molecule comprising a first domain that binds to a first target molecule, thereby activating the iNKT cell, wherein the first target molecule is TRBV25 or TRAV10.

In some embodiments of any method disclosed above, the iNKT cell expresses a T cell receptor beta variable 25 (TRBV25) and/or a T cell receptor alpha variable 10 (TRAV10). In some embodiments of any method disclosed above, the iNKT cell expresses a T cell receptor beta variable 25 (TRBV25) and a T cell receptor alpha variable 10 (TRAV10).

In some embodiments, the iNKT cell is CD4⁺, CD8⁺ or CD4⁻/CD8⁺. In some embodiments, the iNKT cell is CD4⁺. In some embodiments, the iNKT cell is CD8⁺. In some embodiments, the iNKT cell is CD4⁻/CD8⁻. In some embodiments, the iNKT cell is iNKT1, iNKT2, iNKT17, iNKT10, iNKT_{FH}, or any combination thereof. In some embodiments, the iNKT cell is iNKT1. In some embodiments, the iNKT cell is iNKT2. In some embodiments, the iNKT cell is iNKT17. In some embodiments, the iNKT cell is iNKT10. In some embodiments, the iNKT cell is iNKT_{FH}. In some embodiments, the iNKT cell is iNKTreg.

In some embodiments, the NKT cell is obtained from a healthy subject. In some embodiments, the NKT cell is obtained from a subject having a disease. e.g., a cancer, e.g., a solid tumor, as described herein.

In some embodiments of any method disclosed above, the method is performed in vitro or ex vivo. In some embodiments, the method is performed in vivo. In some embodiments, the method is performed ex vivo.

In some embodiments of any methods disclosed above, the method comprises contacting the population of cells comprising the NKT cells with any of the compositions disclosed above for at least about 4 hours, at least 6 hours, at least 10 hours, at least 12 hours, at least 15 hours, at least 18 hours, at least 20 hours, or at least 22 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least about 1 week, at least about 2 weeks, at least about 3 weeks, at least about 4 weeks, at least about 5 weeks, at least about 6 weeks, at least about 7 weeks or at least about 8 weeks.

In some embodiments of any methods disclosed above, the method further comprises contacting the population of cells with an agent, that promote or increases NKT cell activation/expansion. In some embodiments, the agent comprises an immune checkpoint inhibitor. Exemplary immune checkpoint inhibitors including but are not limited to a PD-1 inhibitor, a BTLA inhibitor, a LAG-3 inhibitor, and a CTLA4 inhibitor. In some embodiments, the agent comprises a CD44 agonist, a CD161 agonist, 4-1BB agonist, a ICOS agonist, a CD40L agonist, and a CD28 agonist. In some embodiments, each of the inhibitors or agonists disclosed herein is an antibody.

In some embodiments, the method further comprises comprising contacting the population of cells with a non-dividing population of cells, e.g., feeder cells, e.g., irradiated allogenic human PBMCs.

In some embodiments of any methods disclosed above, the method comprises expanding the iNKT cell at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, 2-fold, at least 3-fold, at least 4-fold, at least 5-fold, at least 6-fold, at least-7 fold, at least-8 fold, at least-9 fold, at least 10-fold, at least 20-fold, at least 30-fold, at least-40 fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, at least 1000 fold, at least 5000-fold, or at least 10000-fold.

In some embodiments of any method disclosed above, the method comprises selectively expanding the iNKT cell. In some embodiments, the method selectively or preferentially expands NKT cells expressing TRAV10 and/or TRBV25 over immune cells that do not express TRAV10 and/or TRBV25 (e.g., T cells expressing TRAV other than TRAV10 or TRBV other than TRBV25). In some embodiments of any method disclosed above, the method comprises expanding the iNKT cell, thereby producing an expanded cell population comprising the expanded iNKT cells, wherein at least at least 1.1%, at least 1.2%, at least 1.3%, at least 1.4%, at least 1.5%, at least 1.6%, at least 1.7%, at least 1.8%, at least 1.9%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the cells in the expanded population of cells are iNKT cells.

In some embodiments of any method disclosed above, the method specifically activates the iNKT cell of the population of cells.

In some embodiments of any method disclosed above, the method comprises activating the iNKT cell, thereby producing an activated cell population comprising the activated iNKT cells, wherein the activated cell population produces a higher level of IL-2, IL-4, IL-13, IL-17A, IL-17RB, TNF-α, or IFN-γ compared to the level of the IL-2, IL-4, IL-13, IL-17A, IL-17RB, TNF-α, or IFN-γ produced by a corresponding cell population produced by contacting the corresponding population of cells comprising the iNKT cell with an anti-CD3 antibody at the same concentration, an anti-TRBV antibody that does not bind TRBV25 at the same concentration, or an anti-TRAV antibody that does not bind TRAV10 at the same concentration according to a cytokine release assay, and/or

In some embodiments, the activated cell population produces a higher level of IL-2, IL-4, IL-13, IL-17A, IL-17RB, TNF-α, or IFN-γ compared to the level of the IL-2, IL-4, IL-13, IL-17A, IL-17RB, TNF-α, or IFN-γ produced by a corresponding cell population produced by contacting the corresponding population of cells comprising the iNKT cell with an anti-CD3 antibody at the same concentration.

In some embodiments, the activated cell population produces a higher level of IL-2, IL-4, IL-13, IL-17A, IL-17RB, TNF-α, or IFN-γ compared to the level of the IL-2, IL-4, IL-13, IL-17A, IL-17RB, TNF-α, or IFN-γ produced by a corresponding cell population produced by contacting the corresponding population of cells comprising the iNKT cell with an anti-TRBV antibody that does not bind TRBV25 at the same concentration.

In some embodiments, the activated cell population produces a higher level of IL-2, IL-4, IL-13, IL-17A, IL-17RB, TNF-α, or IFN-γ compared to the level of the IL-2, IL-4, IL-13, IL-17A, IL-17RB, TNF-α, or IFN-γ produced by a corresponding cell population produced by contacting the corresponding population of cells comprising the iNKT cell with an anti-TRAV antibody that does not bind TRAV10 at the same concentration.

### Methods of Treatment of the Present Technology

In another aspect, provided herein is a method of treating a disease or condition in a subject in need thereof comprising administering to the subject a therapeutically effective amount of any composition disclosed herein.

In another aspect, provided herein is a method of treating a disease or condition in a subject in need thereof comprising administering to the subject a therapeutically effective amount of a composition comprising a molecule comprising a first domain that binds to a first target molecule, wherein the first target molecule is TRBV25 or TRAV10.

In some embodiments, the disease or condition is cancer. In some embodiments, the cancer is a solid tumor, a hematological cancer, a metastatic cancer, a soft tissue tumor, or any combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and any combination thereof. In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof.

In some embodiments of any method of treating a disease or condition disclosed herein, the method further comprises administering a second therapeutic agent or therapy to the subject.

In some embodiments, the second therapeutic agent or therapy comprises a chemotherapeutic agent, a biologic agent, a hormonal therapy, radiation, or surgery. In some embodiments, the second therapeutic agent or therapy is administered in combination with the composition sequentially, simultaneously, or concurrently.

In some embodiments, the disease or condition is an autoimmune disease.

In some embodiments, the autoimmune diseases is selected from the group consisting of multiple sclerosis, Crohn's disease, Sjögren's syndrome, celiac disease, diabetes, arthritis, and Lupus, systemic sclerosis, osteopetrosis, inherited metabolic disorders, adrenoleukodystrophy, amegakaryocytic thrombocytopenia, sickle cell disease, severe congenital immunodeficiency, Griscelli syndrome type II, Hurler syndrome, Kostmann syndrome. Krabbe disease, metachromatic leukodystrophy, thalassemia, hemophagocytic lymphohistiocytosis, and Wiskott-Aldrich syndrome.

In some embodiments, the disease or condition is an infection.

In some embodiments, the infection is a bacterial infection or viral infection.

In some embodiments, the infection is selected from the group consisting of a S. parathyphi infection, a Bacteroidetes infection, a Proteobacteria infection, a M. tuberculosis infection, a respiratory virus infection, a SARS-CoV-2 infection, yellow fever, influenza, a HIV infection, a Cytomegalovirus infection, and an Epstein-Barr Virus infection.

### More Description of Multispecific Antibody Molecules of the Present Technology

In some embodiments, the multispecific molecule comprises a full antibody or fragment thereof (e.g., a Fab, F(ab′)2, Fv, or a single chain Fv fragment (scFv)), i.e., an antibody molecule, such as an anti-TRAV10 antibody or fragment thereof, an anti TRBV25 antibody or fragment thereof, or an antibody or fragment that binds to TAA. In some embodiments, the antibody molecule can be a humanized, chimeric, camelid, shark, or an in vitro-generated antibody molecule. In some embodiments, the antibody molecule is a humanized antibody molecule. The heavy and light chains of the antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the light or the heavy chain variable framework (e.g., the region encompassing at least FRI, FR2, FR3, and optionally FR4) of the antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FRI, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgGI or IgG2 (e.g., human IgGI, or IgG2). In some embodiments, the heavy chain constant region is human IgGI. In some embodiments, the Fc region comprises a Fc region variant, e.g., as described herein.

In some embodiments, the antibody molecule has a light chain constant region chosen from, e.g., the light chain constant regions of kappa or lambda, preferably kappa (e.g., human kappa). In some embodiments, the constant region is altered, e.g., mutated, to modify the properties of the antibody molecule, (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding, e.g., relative to human IgGI.

### Antibody-Like Frameworks or Scaffolds

A wide variety of antibody/immunoglobulin frameworks or scaffolds can be employed in the antibody molecules as described herein or multispecific formats thereof so long as the resulting polypeptide includes at least one binding region which specifically binds to the target antigen, e.g., a TCAV10, TRBV25, a TAA, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the antibody molecules as described herein or multispecific formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRvb or a tumor antigen). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

Fibronectin scaffolds are typically based on fibronectin type III domain (e.g., the tenth module of the fibronectin type 111 (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the disclosure using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the antibody molecules as described herein or multispecific formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254. WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

Antibody Fc domain and Constant Regions

In some embodiments, an antibody as described herein comprises an Fc region, e.g., the first Fc region and the second Fc region as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an antibody comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an antibody comprising a variant Fc region has reduced affinity to a human Fc receptor, e.g., FcγR 1, FcγR II and/or FcγR III. In some embodiments, the antibody comprising a variant Fc region comprises a human IgGI region or a human IgG4 region.

In some embodiments, an antibody comprising a variant Fc region activates and/or expands T cells (e.g., iNKT cells) as described herein.

Exemplary Fc region variants are provided in Table 8 and also disclosed in Saunders 0, (2019) Frontiers in Immunology; vol 10, article1296, the entire contents of which is hereby incorporated by reference. In some embodiments, an antibody as described herein comprises any one or all, or any combination of Fc region variants disclosed in Table 8. In some embodiments, an antibody as described herein comprises any one or all, or any combination of Fc region variants, e.g., mutations, disclosed in Table 8. In some embodiments, an antibody as described herein comprise an Asn297Ala (N297A) mutation. In some embodiments, an antibody as described herein comprise a Leu234Ala/Leu235Ala (LALA) mutation.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold. 9 fold, 10 fold, 15 fold, 20 fold. 25 fold, 30 fold, 35 fold, 40 fold, 45 fold, 50 fold, 55 fold. 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold. 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold. 300 fold, 250 fold, 400 fold, 450 fold. 500 fold, 550 fold, 600 fold, 650 fold, 700 fold, 750 fold, 800 fold, 850 fold, 900 fold, 950 fold, 1000 fold, 2000 fold, 3000 fold, 4000 fold. 5000 fold, 6000 fold, 7000 fold. 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold. 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 430, SEQ ID NO: 432, SEQ ID NO: 437, SEQ ID NO: 438, SEQ ID NO: 439, SEQ ID NO: 40, or SEQ ID NO: 441. In some embodiments, the first Fc region, the second Fc region, or a combination thereof comprises the sequence of SEQ ID NO: 430, SEQ ID NO: 432, SEQ ID NO: 437, SEQ ID NO: 438, SEQ ID NO: 439, SEQ ID NO: 440, or SEQ ID NO: 441.

In some embodiments, the first polypeptide, the second polypeptide, or a combination thereof comprises a heavy chain constant region having a sequence having at least 75%, 80%, 85%, 90%, 95%, 99%, 99.5%, 99.9%, or 100% sequence identity to any one of the sequences listed in Table 20 or a combination thereof. In some embodiments, the first polypeptide, the second polypeptide, or a combination thereof comprises a heavy chain constant region having any one of the sequences listed in Table 20 or a combination thereof. In some embodiments, the first polypeptide, the second polypeptide, or a combination thereof comprises a heavy chain constant region of an IgM or a fragment thereof. In some embodiments, the heavy chain constant region of the IgM comprises a sequence having at least 75%, 80%, 85%, 90%, 95%, 99%. 99.5%, 99.9%, or 100% sequence identity to the sequence of SEQ ID NO: 433. In some embodiments, the heavy chain constant region of the IgM comprises the sequence of SEQ ID NO: 433. In some embodiments, the sequence of the heavy chain constant region of the IgM is the sequence of SEQ ID NO: 433.

TABLE 20
Constant region amino acid sequences of human IgG heavy chains and human kappa
light chain

| | | |
|---|---|---|
| Human kappa | LC | RTVAAPSVFI FPPSDEQLKS GTASVVCLLN NFYPREAKVQ |
| constant region | | WKVDNALQSG NSQESVTEQD SKDSTYSLSS TLTLSKADYE |
| SEQ ID NO: | | KHKVYACEVT HQGLSSPVTK SFNRGEC |
| 428 | | |
| Human | LC | RTVAAPSVFIFPPSDEQLKSGTASVVCLLNNFYPREAKVQWKVD |
| Immunoglobulin | | NALQSGNSQESVTEQDSKDSTYSLSSTLTLSKADYEKHKVYACE |
| kappa | | VTHQGLSSPVTKSFNRGEC |
| constant C | | |
| region | | |
| SEQ ID NO: | | |
| 429 | | |
| IgG4 (S228P) | HC | ASTKGPSVFPLAPCSRSTSESTAALGCLVKDYFPEPVTVSWNSGA |
| mutant | | LTSGVHTFPAVLQSSGLYSLSSVVTVPSSSLGTKTYTCNVDHKPS |
| constant region | | NTKVDKRVESKYGPPCPPCPAPEFLGGPSVFLFPPKPKDTLMISRT |
| (EU | | PEVTCVVVDVSQEDPEVQFNWYVDGVEVHNAKTKPREEQFNST |
| Numbering) | | YRVVSVLTVLHQDWLNGKEYKCKVSNKGLPSSIEKTISKAKGQP |
| SEQ ID NO: | | REPQVYTLPPSQEEMTKNQVSLTCLVKGFYPSDIAVEWESNGQP |
| 430 | | ENNYKTTPPVLDSDGSFFLYSRLTVDKSRWQEGNVFSCSVMHEA |
| | | LHNHYTQKSLSLSLG |
| IgG1 wild type | HC | ASTKGPSVFPLAPSSKSTSGGTAALGCLVKDYFPEPVTVSWNSG |
| SEQ ID NO: | | ALTSGVHTFPAVLQSSGLYSLSSVVTVPSSSLGTQTYICNVNHKP |
| 431 | | SNTKVDKRVEPKSCDKTHTCPPCPAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| IgG1 (N297A) | HC | ASTKGPSVFPLAPSSKSTSGGTAALGCLVKDYFPEPVTVSWNSG |
| mutant | | ALTSGVHTFPAVLQSSGLYSLSSVVTVPSSSLGTQTYICNVNHKP |
| constant region | | SNTKVDKRVEPKSCDKTHTCPPCPAPELLGGPSVFLFPPKPKDTL |
| (EU | | MISRTPEVTCVVVDVSHEDPEVKFNWYVDGVEVHNAKTKPREE |
| Numbering) | | QYASTYRVVSVLTVLHQDWLNGKEYKCKVSNKALPAPIEKTISK |
| SEQ ID NO: | | AKGQPREPQVYTLPPSREEMTKNQVSLTCLVKGFYPSDIAVEWE |
| 432 | | SNGQPENNYKTTPPVLDSDGSFFLYSKLTVDKSRWQQGNVFSCS |
| | | VMHEALHNHYTQKSLSLSPGK |
| IgM constant | HC | GSASAPTLFPLVSCENSPSDTSSVAVGCLAQDFLPDSITFSWKYK |
| delta CDC | | NNSDISSTRGFPSVLRGGKYAATSQVLLPSKDVMQGTDEHVVCK |
| (P311A, P313S) | | VQHPNGNKEKNVPLPVIAELPPKVSVFVPPRDGFFGNPRKSKLIC |
| SEQ ID NO: | | QATGFSPRQIQVSWLREGKQVGSGVTTDQVQAEAKESGPTTYK |
| 433 | | VTSTLTIKESDWLGQSMFTCRVDHRGLTFQQNASSMCVPDQDT |
| | | AIRVFAIPPSFASIFLTKSTKLTCLVTDLTTYDSVTISWTRQNGEA |
| | | VKTHTNISESHPNATFSAVGEASICEDDWNSGERFTCTVTHTDLA |
| | | SSLKQTISRPKGVALHRPDVYLLPPAREQLNLRESATITCLVTGFS |
| | | PADVFVQWMQRGQPLSPEKYVTSAPMPEPQAPGRYFAHSILTVS |
| | | EEEWNTGETYTCVVAHEALPNRVTERTVDKSTGKPTLYNVSLV |
| | | MSDTAGTCY |
| IgGA1 | HC | ASPTSPKVFPLSLCSTQPDGNVVIACLVQGFFPQEPLSVTWSESG |
| SEQ ID NO: | | QGVTARNFPPSQDASGDLYTTSSQLTLPATQCLAGKSVTCHVKH |
| 434 | | YTNPSQDVTVPCPVPSTPPTPSPSTPPTPSPSCCHPRLSLHRPALED |
| | | LLLGSEANLTCTLTGLRDASGVTFTWTPSSGKSAVQGPPERDLC |
| | | GCYSVSSVLPGCAEPWNHGKTFTCTAAYPESKTPLTATLSKSGN |
| | | TFRPEVHLLPPPSEELALNELVTLTCLARGFSPKDVLVRWLQGSQ |
| | | ELPREKYLTWASRQEPSQGTTTFAVTSILRVAAEDWKKGDTFSC |
| | | MVGHEALPLAFTQKTIDRLAGKPTHVNVSVVMAEVDGTCY |
| IgGA2 | HC | ASPTSPKVFPLSLDSTPQDGNVVVACLVQGFFPQEPLSVTWSESG |
| SEQ ID NO: | | QNVTARNFPPSQDASGDLYTTSSQLTLPATQCPDGKSVTCHVKH |
| 435 | | YTNSSQDVTVPCRVPPPPPCCHPRLSLHRPALEDLLLGSEANLTC |
| | | TLTGLRDASGATFTWTPSSGKSAVQGPPERDLCGCYSVSSVLPG |
| | | CAQPWNHGETFTCTAAHPELKTPLTANITKSGNTFRPEVHLLPPP |
| | | SEELALNELVTLTCLARGFSPKDVLVRWLQGSQELPREKYLTWA |
| | | SRQEPSQGTTTYAVTSILRVAAEDWKKGETFSCMVGHEALPLAF |
| | | TQKTIDRMAGKPTHINVSVVMAEADGTCY |
| Human Ig_J | HC | MKNHLLFWGVLAVFIKAVHVKAQEDERIVLVDNKCKCARITSRI |
| chain | | IRSSEDPNEDIVERNIRIIVPLNNRENISDPTSPLRTRFVYHLSDLCK |
| SEQ ID NO: | | KCDPTEVELDNQIVTATQSNICDEDSATETCYTYDRNKCYTAVV |
| 436 | | PLVYGGETKMVETALTPDACYPD |
| Human | HC | ASTKGPSVFPLAPSSKSTSGGTAALGCLVKDYFPEPVTVSWNSG |
| Immunoglobulin | | ALTSGVHTFPAVLQSSGLYSLSSVVTVPSSSLGTQTYICNVNHKP |
| heavy chain | | SNTKVDKKVEPKSCDKTHTCPPCPAPELLGGPSVFLFPPKPKDTL |
| SEQ ID NO: | | MISRTPEVTCVVVDVSHEDPEVKFNWYVDGVEVHNAKTKPREE |
| 437 | | QYASTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| Human | HC | ASTKGPSVFPLAPSSKSTSGGTAALGCLVKDYFPEPVTVSWNSG |
| Immunoglobulin | | ALTSGVHTFPAVLQSSGLYSLSSVVTVPSSSLGTQTYICNVNHKP |
| heavy chain | | SNTKVDKKVEPKSCDKTHTCPPCPAPELLGGPSVFLFPPKPKDTL |
| SEQ ID NO: | | MISRTPEVTCVVVDVSHEDPEVKFNWYVDGVEVHNAKTKPREE |
| 438 | | QYASTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGKEPEA |
| Human | HC | ASTKGPSVFPLAPSSKSTSGGTAALGCLVKDYFPEPVTVSWNSG |
| Immunoglobulin | | ALTSGVHTFPAVLQSSGLYSLSSVVTVPSSSLGTQTYICNVNHKP |
| heavy chain | | SNTKVDKRVEPKSCDKTHTCPPCPAPELLGGPSVFLFPPKPKDTL |
| SEQ ID NO: | | MISRTPEVTCVVVDVSHEDPEVKFNWYVDGVEVHNAKTKPREE |
| 439 | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| Human Fc | HC | DKTHTCPPCPAPELLGGPSVFLFPPKPKDTLMISRTPEVTCVVVD |
| Knob cys | | VSHEDPEVKFNWYVDGVEVHNAKTKPREEQYASTYRVVSVLTV |
| N297A | | LHQDWLNGKEYKCKVSNKALPAPIEKTISKAKGQPREPQVYTLP |
| SEQ ID NO: | | PCREEMTKNQVSLWCLVKGFYPSDIAVEWESNGQPENNYKTTPP |
| 440 | | VLDSDGSFFLYSKLTVDKSRWQQGNVFSCSVMHEALHNHYTQK |
| | | SLSLSPGK |
| Human IgG1 | HC | ASTKGPSVFPLAPSSKSTSGGTAALGCLVKDYFPEPVTVSWNSG |
| hole cys | | ALTSGVHTFPAVLQSSGLYSLSSVVTVPSSSLGTQTYICNVNHKP |
| N297A | | SNTKVDKRVEPKSCDKTHTCPPCPAPELLGGPSVFLFPPKPKDTL |
| SEQ ID NO: | | MISRTPEVTCVVVDVSHEDPEVKFNWYVDGVEVHNAKTKPREE |
| 441 | | QYASTYRVVSVLTVLHQDWLNGKEYKCKVSNKALPAPIEKTISK |
| | | AKGQPREPQVCTLPPSREEMTKNQVSLSCAVKGFYPSDIAVEWE |
| | | SNGQPENNYKTTPPVLDSDGSFFLVSKLTVDKSRWQQGNVFSCS |
| | | VMHEALHNHYTQKSLSLSPGK |

In some embodiments, the first polypeptide, the second polypeptide, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100/% sequence identity to the sequence of SEQ ID NO: 436. In some embodiments, the heavy chain constant region of the IgJ comprises the sequence of SEQ ID NO: 436. In some embodiments, the sequence of the heavy chain constant region of the IgJ is the sequence of SEQ ID NO: 436.

In some embodiments, the first polypeptide, the second polypeptide, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 434. In some embodiments, the heavy chain constant region of the IgGA1 comprises the sequence of SEQ ID NO: 434. In some embodiments, the sequence of the heavy chain constant region of the IgGA1 is the sequence of SEQ ID NO: 434.

In some embodiments, the first polypeptide, the second polypeptide, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5%, 99.9%, or 100% sequence identity to the sequence of SEQ ID NO: 435. In some embodiments, the heavy chain constant region of the IgGA2 comprises the sequence of SEQ ID NO: 435. In some embodiments, the sequence of the heavy chain constant region of the IgGA2 is the sequence of SEQ ID NO: 435.

In some embodiments, the first polypeptide, the second polypeptide, or a combination thereof comprises a heavy chain constant region of an IgGI or a fragment thereof. In some embodiments, the heavy chain constant region of the IgGI comprises a sequence having at least 75%, 80%, 85%, 90%, 95%, 99%, 99.5%, 99.9%, or 100% sequence identity to the sequence of SEQ ID NO: 431. In some embodiments, the heavy chain constant region of the IgG1 comprises the sequence of SEQ ID NO: 431. In some embodiments, the sequence of the heavy chain constant region of the IgGI is the sequence of SEQ ID NO: 431. In some embodiments, the heavy chain constant region of the IgGI comprises a sequence having at least 75%, 80%, 85%, 90%, 95%, 99%, 99.5%, 99.9%, or 100% sequence identity to the sequence of SEQ ID NO: 437. In some embodiments, the heavy chain constant region of the IgGI comprises the sequence of SEQ ID NO: 437. In some embodiments, the sequence of the heavy chain constant region of the IgGI is the sequence of SEQ ID NO: 437.

In some embodiments, the third polypeptide, the fourth polypeptide, the fifth polypeptide, the sixth polypeptide, or a combination thereof comprises a light chain constant region having a sequence having at least 75%, 80%, 85%, 90%, 95%, 99%, 99.5%, 99.9%, or 100% sequence identity to any one of the sequences listed in Table 20 or a combination thereof. In some embodiments, the third polypeptide, the fourth polypeptide, the fifth polypeptide, the sixth polypeptide, or a combination thereof comprises a light chain constant region having any one of the sequences listed in Table 20 or a combination thereof. In some embodiments, the third polypeptide, the fourth polypeptide, the fifth polypeptide, the sixth polypeptide, or a combination thereof comprises a light chain constant region having any one of the light chain constant region sequences listed in Table 20 or a combination thereof.

In some embodiments, the third polypeptide, the fourth polypeptide, the fifth polypeptide, the sixth polypeptide, or a combination thereof comprises a light chain constant region of a kappa chain or a fragment thereof. In some embodiments, the light chain constant region of a kappa chain comprises a light chain constant region sequence listed in Table 20.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 428 or SEQ ID NO: 429. In some embodiments, the light chain constant region of a kappa chain comprises the sequence of SEQ ID NO: 428 or SEQ 1D NO: 429. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 428 or SEQ ID NO: 429. In some embodiments, the sequence of the light chain constant region of a kappa chain is the sequence of SEQ ID NO: 428 or SEQ ID NO: 429.

### Multispecific Antibody Molecules

Exemplary structures of multispecific molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In some embodiments, multispecific antibody molecules can comprise more than one antigen-binding site, where different sites are specific for different antigens. In some embodiments, multispecific antibody molecules can bind more than one (e.g., two or more) epitopes on the same antigen. In some embodiments, multispecific antibody molecules comprise an antigen-binding site specific for a target cell (e.g., cancer cell) and a different antigen-binding site specific for an immune effector cell. In some embodiments, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, Kk-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in Kk-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-la and IL-10; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., K col). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### Linkers

The multispecific molecule as described herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Ser. In some embodiments, the peptide linker is selected from GGGGS (SEQ ID NO: 442); GGGGSGGGGS (SEQ ID NO: 443); GGGGSGGGGSGGGGS (SEQ ID NO: 444); DVPSGPGGGGGSGGGGS (SEQ ID NO: 445); and GGGGSGGGGSGGGGGS (SEQ ID NO: 446). In some embodiments, the peptide linker is a A(EAAAK)nA (SEQ ID NO: 447) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 448); AEAAAKEAAAKEAAAKAAA (SEQ ID NO: 449); AEAAAKEAAAKEAAAKEAAAKAAA (SEQ ID NO: 450); and AEAAAKEAAAKEAAAKEAAAKEAAAKAAA(SEQ ID NO: 451).

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multispecific polypeptide molecule as described herein.

Nucleic acids encoding the aforementioned antibody molecules, e.g., anti-TCRαV antibody molecules, multispecific molecules are also disclosed.

Nucleic acids encoding the aforementioned recombinant T cell receptor or CAR are also disclosed.

In certain embodiments, the disclosure features nucleic acids comprising nucleotide sequences that encode heavy and light chain variable regions and CDRs or hypervariable loops of the antibody molecules, as described herein. For example, the disclosure features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRαV antibody molecules, a multispecific molecule, or a CAR described herein. In some embodiments, the vectors comprise nucleic acid sequences encoding antibody molecules, e.g., anti-TCRαV antibody molecules, multispecific, or a CAR described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Pharmaceutical Compositions

Described herein, in certain embodiments, is a pharmaceutical composition comprising any multispecific molecule as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multispecific molecules or the compositions as described herein, or a pharmaceutically acceptable salt, solvate, hydrate or ester thereof. The pharmaceutical formulation comprising the multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate. 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes. e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multispecific molecules as described herein. In some embodiments, the multispecific molecules or the compositions as described herein is administered in combination with another drug or therapeutic agent.

### Administration

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multispecific molecules or the compositions as described herein to the fetus directly or indirectly (e.g., via the mother).

Suitable routes for administration of the multispecific molecules or the compositions as described herein may vary depending on cell type to which delivery of the multispecific molecules or the compositions is desired. The multispecific molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multispecific molecules or the compositions as described herein are administered with one or more agents capable of promoting penetration of the subject the multispecific molecules or the compositions as described herein across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the multispecific molecules or the compositions as described herein are linked or conjugated with agents that provide desirable pharmaceutical or pharmacodynamic properties. In some embodiments, the multispecific molecules or the compositions as described herein are coupled to a substance, known in the art to promote penetration or transport across the blood-brain barrier, e.g., an antibody to the transferrin receptor. In some embodiments, the multispecific molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Combination Therapy

In some embodiments, the multispecific molecules as described herein is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®)), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATNOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific molecules as described herein is administered in conjunction with a biologic. Biologics useful in the treatment of cancers are known in the art and a binding molecule as described herein may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals. LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer): FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules as described herein may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride. Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® r (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In some embodiments, the multispecific molecules as described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299. JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In some embodiments, the multispecific molecules as described herein are administered in combination with a nano pharmaceutical. Exemplary cancer nano pharmaceuticals include, but not limited to, ABRAXANE® (paclitaxel bound albumin nanoparticles), CRLX101 (CPT conjugated to a linear cyclodextrin-based polymer). CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific molecules as described herein are administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Boorganic & Medicinal Chemistry Letters (2007) 17:617-620).

Exemplary RNAi and antisense RNA agents for treating cancer include, but not limited to, CALAA-01, siG12D LODER (Local Drug Elute®), and ALN-VSP02.

Other cancer therapeutic agents include, but not limited to, cytokines (e.g., aldesleukin (IL-2, Interleukin-2, PROLEUKIN®), alpha Interferon (IFN-alpha. Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®)), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®), VIADUR®), megestrol (megestrol acetate, MEGACE®), nilutamide (ANANDRON®, NILANDRON®), octreotide (octreotide acetate. SANDOSTATIN®, SANDOSTATIN LAR®), raloxifene (EVISTA®), romiplostim (NPLATE®)), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK® 10), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL4), prednisolone (DELTA-CORTEFS, ORAPRED®, PEDIAPRED®, PRELONEt), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the multispecific molecules as described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of: axitinib (AGO13736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®)), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010). MGCD-265, motesanib diphosphate (AMG-706), MP-470, OSI-930, Pazopanib Hydrochloride, PD173074, Sorafenib Tosylate (Bay 43-9006), SU 5402, TSU-68(SU6668), vatalanib, XL880 (GSK1363089. EXEL-2880). Selected tyrosine kinase inhibitors are chosen from sunitinib, erlotinib, gefitinib, or sorafenib. In some embodiments, the tyrosine kinase inhibitor is sunitinib.

In some embodiments, the multispecific molecules as described herein are administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab): VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

In other embodiments, methods described herein comprise use of an immune checkpoint inhibitor in combination with the multispecific molecules as described herein. The methods can be used in a therapeutic protocol in vivo.

In some embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class 1, MHC class II, GAL9, VISTA, BTLA, TIGIT, LAIR1, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475. SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In some embodiments, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In some embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In some embodiments, the PD-L1 inhibitor is an anti-PD-L1 antibody, e.g., YW243.55.S70. The YW243.55.570 antibody is described, e.g., in WO 2010/077634. In some embodiments, the PD-L1 inhibitor is MDX-1105 (also called BMS-936559), which is described, e.g., in WO2007/005874. In some embodiments, the PD-LI inhibitor is MDPL3280A (Genentech/Roche), which is a human Fc-optimized IgGI monoclonal antibody against PD-L1. See, e.g., U.S. Pat. No. 7,943,743 and U.S Publication No.: 20120039906. In some embodiments, the inhibitor of PD-L1 is an antibody molecule having a sequence substantially identical or similar thereto, e.g., a sequence at least 85%, 90° %, 95% identical or higher to the sequence of YW243.55.570, MPDL3280A, MEDI-4736, MSB-0010718C, or MDX-1105.

In some embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In some embodiments, the immune checkpoint inhibitor is a LAG-3 inhibitor, e.g., an anti-LAG-3 antibody molecule. In some embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### CRS Grading

As noted above, an advantage of the composition and methods disclosed herein is reduced Cytokine Release Syndrome (CRS). In some embodiments, CRS (Cytokine Release Syndrome) can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Tables 5-7.

### Part III

### Anti-TCRβV Antibodies

### Human T Cell Receptor (TCR) Complex

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCR. V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily. TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, as well as family members of said subfamilies, and variants thereof (e.g., a structural or functional variant thereof). In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRBV comprises TCRβ V6-5*01, or a variant thereof, e.g., a variant having 85%, 90%, 95%, 99% or more identity the naturally-occurring sequence. TCRβ V6-5*01 is also known as TCRvβ65; TCRvβ6S5; TCRvρ313S1, or TCRβ V13.1. The amino acid sequence of TCRβ V6-5*01, e.g., human TCRβ V6-5*01, is known in that art, e.g., as provided by IMGT ID L36092. In some embodiments, TCRβ V6-5*01 is encoded by the nucleic acid sequence of SEQ ID NO: 1043, or a sequence having 85%, 90%, 95%, 99% or more identity thereof. In some embodiments, TCRβ V6-5*01 comprises the amino acid sequence of SEQ ID NO: 1044, or a sequence having 85%, 90%, 95%, 99% or more identity thereof.

SEQ ID NO: 1043
ATGAGCATCGGCCTCCTGTGCTGTGCAGCCTTGTCTCTCCTGTGGGCAG
GTCCAGTGAATGCTGGTGTCACTCAGACCCCAAAATTCCAGGTCCTGAA
GACAGGACAGAGCATGACACTGCAGTGTGCCCAGGATATGAACCATGAA
TACATGTCCTGGTATCGACAAGACCCAGGCATGGGGCTGAGGCTGATTC
ATTACTCAGTTGGTGCTGGTATCACTGACCAAGGAGAAGTCCCCAATGG
CTACAATGTCTCCAGATCAACCACAGAGGATTTCCCGCTCAGGCTGCTG
TCGGCTGCTCCCTCCCAGACATCTGTGTACTTCTGTGCCAGCAGTTACTC
SEQ ID NO: 1044
MSIGLLCCAALSLLWAGPVNAGVTQTPKFQVLKTGQSMTLQCAQDMNHE
YMSWYRQDPGMGLRLIHY-SVGAGITDQGEVPNGYNVSRSTTEDFPLRL
LSAAPSQTSVYFCASSY

In some embodiments, the TCRβ V4 subfamily comprises: TCRβ V4-1*01. In some embodiments, TCRβV comprises TCRβ V4-1*01, or a variant thereof, e.g., a variant having 85%, 90%, 95%, 99% or more identity the naturally-occurring sequence. TCR beta V (TCRβV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

Provided herein are, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7)pp: 493-502; Wei S, and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRBV, TCRVB, TRBV, TCRβV, TCRVβ or TRβV are used interchangeably herein and refer to a TCR beta V chain, e.g., as described herein.

In some embodiments, provided herein is an anti-TCRBV antibody molecule that binds to human TCRβV, e.g., a TCRBV family, e.g., gene family or a variant thereof. In some embodiments a TCRBV gene family comprises one or more subfamilies. e.g., as described herein, e.g., Table 28. In some embodiments, the TCRβV gene family comprises: a TCRβ V4 subfamily, or a TCRβ V6 subfamily.

In some embodiments, the TCRβ V4 subfamily comprises TCRβ V4-2*01, TCRβ V4-3*01, or TCRβ V4-1*01, or a variant thereof.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments. TCRβ V6 comprises TCRβ V6-6*01, or a variant thereof. In some embodiments, TCRβ V6 comprises TCRβ V6-2*01, or a variant thereof. In some embodiments, TCRβ V6 comprises TCR13 V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1001 and/or SEQ ID NO: 1002. In some embodiments, TCRβ V6, e.g., TCRβ V6-5*01, is recognized, e.g., bound, by SEQ ID NO: 1009 and/or SEQ ID NO: 1010. In some embodiments, TCRβ V6 is recognized, e.g., bound, by SEQ ID NO: 1009 and/or SEQ ID NO: 1011.

Exemplary amino acid sequences for TCRβV subfamily members can be found on the ImMunoGeneTics Information System website: http://wwvw.imgt.org/, or in a similar resource.

### Anti-TCRβV Antibodies

Current bispecific constructs designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRβV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRβV antibody molecules as described herein, which despite having low sequence similarity (e.g., low sequence identity among the different antibody molecules that recognize different TCRβV subfamilies), recognize a structurally conserved, yet sequence-wise variable, region, e.g., domain, on the TCRβV protein and have a similar function (e.g., activation of T cells and a similar cytokine profile as described herein). Thus, the anti-TCR3V antibody molecules as described herein share a structure-function relationship.

Without wishing to be bound by theory, in some embodiments, the anti-TCRβV antibody molecules as described herein bind to an outward facing epitope of a TCRβV protein when it is in a complex with a TCRalpha protein. In some embodiments, the anti-TCRβV antibody molecules as described herein recognize (e.g., bind to), a domain (e.g., an epitope) on the TCRβV protein that is: (1) structurally conserved among different TCRβV subfamilies; and (2) has minimal sequence identity among the different TCRβV subfamilies. TCRβV proteins from the different TCRβV subfamilies share minimal sequence similarity. However, TCRβV proteins which have minimal sequence similarity, share a similar 3D conformation and structure.

The alignment of TCRβV amino acid sequences in ta9 underscores the diversity of TCR sequences. In particular, the TRBV sequences from different subfamilies are considerably different from each other.

In some embodiments, the anti-TCRβV antibody molecules as described herein do not recognize, e.g., bind to, an interface of a TCRβV:TCRalpha complex. In some embodiments, the anti-TCRβV antibody molecules as described herein do not recognize, e.g., bind to, a constant region of a TCRβV protein. An exemplary antibody that binds to a constant region of a TCRβV region is JOVI.1 as de-scribed in Viney et al., (Hybridoma. 1992 December; 11(6):701-13). In some embodiments, the anti-TCRβV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the beta subunit of TCR (TCRβV) which bind and, e.g., activate a subset of T cells. The anti-TCRβV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRβV antibodies as described herein have a cytokine profile, e.g., as described herein, which differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in expansion of TCRβV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRβV antibody molecules and uses thereof. Also described herein are multispecific molecules, e.g., bispecific molecules comprising said anti-TCRβV antibody molecules. In some embodiments, compositions comprising anti-TCRβV antibody molecules of the present disclosure, can be used, e.g., to: (1) activate and redirect T cells to promote tumor cell lysis for cancer immuno-therapy; and/or (2) expand TCRβV+ T cells. In some embodiments, compositions comprising anti-TCRβV antibody molecules as described herein limit the harmful side-effects of CRS and/or NT, e.g., CRS and/or NT associated with anti-CD3e targeting.

In some embodiments, the anti-TCRβV antibody molecule binds to one or more of TRBV4-1, TRBV4-2, TRBV4-3, TRBV6-1, TRBV6-2, TRBV6-3, TRBV6-4, TRBV6-5, TRBV6-6, TRBV6-8, and TRBV6-9. In some embodiments, the anti-TCR43V antibody molecule binds to one or more of TRBV6-1, TRBV6-2, TRBV6-3, TRBV6-4, TRBV6-5, TRBV6-6, TRBV6-8 and TRBV6-9. In some embodiments, the anti-TCRβV antibody molecule is an anti-TRBV4-1, anti-TRBV4-2, anti-TRBV4-3, anti-TRBV6-1, anti-TRBV6-2, anti-TRBV6-3, anti-TRBV6-4, anti-TRBV6-5, anti-TRBV6-6, anti-TRBV6-8, or anti-TRBV6-9. Exemplary anti-TCRβV antibody molecules and the corresponding TCRβV subfamilies recognized by said anti-TCRβV antibody molecules are disclosed in Table 29.

In some embodiments, the anti-TCRβV antibody molecule binds specifically to TRBV4-1, TRBV4-2, TRBV4-3, TRBV6-1, TRBV6-2, TRBV6-3, TRBV6-4, TRBV6-5, TRBV6-6, TRBV6-8, or TRBV6-9. In some embodiments, the anti-TCRβV antibody molecule binds specifically to TRBV4-1. In some embodiments, the anti-TCRβV antibody molecule binds specifically to TRBV4-2. In some embodiments, the anti-TCRβV antibody molecule binds specifically to TRBV4-3. In some embodiments, the anti-TCRβV antibody molecule binds specifically to TRBV6-1. In some embodiments, the anti-TCRβV antibody molecule binds specifically to TRBV6-2. In some embodiments, the anti-TCRβV antibody molecule binds specifically to TRBV6-3. In some embodiments, the anti-TCRβV antibody molecule binds specifically to TRBV6-4. In some embodiments, the anti-TCRβV antibody molecule binds specifically to TRBV6-5. In some embodiments, the anti-TCRβV antibody molecule binds specifically to TRBV6-6. In some embodiments, the anti-TCRβV antibody molecule binds specifically to TRBV6-8. In some embodiments, the anti-TCRβV antibody molecule binds specifically to TRBV6-9.

In some embodiments, the anti-TCRβV antibody molecule does not comprise the CDRs of the Antibody B murine antibody.

In some embodiments, the light or the heavy chain variable framework (e.g., the region encompassing at least FRI. FR2, FR3, and optionally FR4) of the anti-TCRβV antibody molecule, e.g., anti-TCRβ V6 (e.g., anti-TCβD V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., in SEQ ID NO: 1009.

Alternatively, or in combination with the heavy chain substitutions described herein, the anti-TCRβV antibody molecule, e.g., anti-TCRβ V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g SEQ ID NO: 1010 or SEQ ID NO: 1011.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 2 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 3 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 4 of A-H.1 or A-H.2.

In some embodiments, the anti-TCRβ V antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenylalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenylalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position as described herein according to Kabat numbering. In some embodiments. FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβ V antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position as described herein according to Kabat numbering. In some embodiments, FR3 comprises a Phenylalanine at position 87, e.g., a substitution at position 87 according to Kabat numbering, e.g., a Tyrosine to Phenylalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a Phenylalanine at position 10, e.g., a substitution at position 10 according to Kabat numbering. e.g., a Serine to Phenylalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenylalanine substitution, e.g., as shown in the amino acid sequence of SEQ ID NO: 1010. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenylalanine substitution, e.g., as shown in the amino acid sequence of SEQ ID NO: 1011. In some embodiments, the substitution is relative to a human germline light chain framework region sequence.

In some embodiments, the anti-TCR4V antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions as described herein according to Kabat numbering; (b) a framework region 2 (FR2) comprising a change, e.g., a substitution (e.g., a conservative substitution) at one or more (e.g., all) position as described herein according to Kabat numbering and (c) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2. In some embodiments, the anti-TCR4V antibody molecule, e.g., anti-TCRβ V6 (e.g., anti-TCRβ V6-5*01) antibody molecule, comprises the heavy chain framework region 3 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position as described herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering. e.g., a Arginine to Glycine substitution, e.g., as shown in the amino acid sequence of SEQ ID NO: 1010.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 1009. In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 1010. In some embodiments, the anti-TCRβV antibody molecule. e.g., anti-TCRβ V6 (e.g., anti-TCRβ V6-5*01) antibody molecule, comprises the light chain framework regions 1-4 of A-H.2, e.g., SEQ ID NO: 1011. In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO: 1009; and the light chain framework regions 1-4 of A-H.1, e.g., SEQ ID NO: 1010. In some embodiments, the anti-TCRβV antibody molecule, e.g., anti-TCRβ V6 (e.g., anti-TCRβ V6-5*01) antibody molecule, comprises the heavy chain framework regions 1-4 of A-H.2, e.g., SEQ ID NO: 1009; and the light chain framework regions 1-4 of A-H.2, e.g., SEQ ID NO: 1011.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in Table 21, or encoded by the nucleotide sequence in Table 21; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 21, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 21. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 21, or a sequence substantially identical thereto (e.g., a sequence at least about 85%, 90%, 95%, 99% or more identical thereto, or which differs by no more than 3, 6, 15, 30, or 45 nucleotides from the sequences shown in Table 21.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 1009, an amino acid sequence at least about 85%, 90%, 95%, 99% or more identical to the amino acid sequence of SEQ ID NO: 1009, or an amino acid sequence which differs by no more than 1, 2, 5, 10, or 15 amino acid residues from the amino acid sequence of SEQ ID NO: 1009; and/or a VL domain comprising the amino acid sequence of SEQ ID NO: 1010, an amino acid sequence at least about 85%, 90%, 95%, 99% or more identical to the amino acid sequence of SEQ ID NO: 1010, or an amino acid sequence which differs by no more than 1, 2, 5, 10, or 15 amino acid residues from the amino acid sequence of SEQ ID NO: 1010.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 1009, an amino acid sequence at least about 85%, 90%, 95%, 99% or more identical to the amino acid sequence of SEQ ID NO: 1009, or an amino acid sequence which differs by no more than 1, 2, 5, 10, or 15 amino acid residues from the amino acid sequence of SEQ ID NO: 1009; and/or a VL domain comprising the amino acid sequence of SEQ ID NO: 1011, an amino acid sequence at least about 85%, 90%, 95%, 99% or more identical to the amino acid sequence of SEQ ID NO: 1011, or an amino acid sequence which differs by no more than 1, 2, 5, 10, or 15 amino acid residues from the amino acid sequence of SEQ ID NO: 1011.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)2, Fv, single domain antibody, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a multispecific molecule, e.g., a bispecific molecule, e.g., as described herein.

In some embodiments, the anti-TCR4V antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgGI or IgG2 (e.g., human IgG1, or IgG2). In some embodiments, the heavy chain constant region is human IgGI. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 1212 or 1214; or positions 135 (M to Y), 137 (S to T), 139 (T to E), 316 (H to K) and 317 (N to F) of SEQ ID NOs: 1215, 1216, 1217 or 1218), e.g., relative to human IgGI.

Antibody A-H.1 comprises a heavy chain comprising the amino acid sequence of SEQ ID NO: 1020 and a light chain comprising the amino acid sequence of SEQ ID NO: 1023. Antibody A-H.2 comprises a heavy chain comprising the amino acid sequence of SEQ ID NO: 1020 and a light chain comprising the amino acid sequence of SEQ ID NO: 1021. Antibody A-H.68 comprises the amino acid sequence of SEQ ID NO: 1161, or a sequence having at least 85%, 90%, 95%, 96%, 97%, 98%, 99%, 99.5%, 99.9%, or 100% sequence identity thereto. Antibody A-H.69 comprises the amino acid sequence of SEQ ID NO: 1060, or a sequence having at least 85%, 90%, 95%, 96%, 97%, 98%, 99%, 99.5%, 99.9%, or 100% sequence identity thereto.

Additional exemplary humanized anti-TCRβ V6 antibodies are provided in Table 21. In some embodiments, the anti-TCRβ V6 is antibody A, e.g., humanized antibody A (antibody A-H), as provided in Table 21. In some embodiments, the anti-TCRβV antibody comprises one or more (e.g., all three) of a LC CDR1, LC CDR2, and LC CDR3 provided in Table 21; and/or one or more (e.g., all three) of a HC CDR1, HC CDR2, and HC CDR3 provided in Table 21, or a sequence with at least 85%, 90%, 95%, 96%, 97%, 98%, 99%, 99.5%, 99.9%, or 100% sequence identity thereto. In some embodiments, antibody A comprises a variable heavy chain (VH) and/or a variable light chain (VL) provided in Table 21, or a sequence with at least 85%, 90° %, 95%, 96%, 97%, 98%, 99%, 99.5%, 99.9%, or 100% sequence identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

Exemplary anti-TCRβV antibody molecules and the corresponding TCR43V subfamilies recognized by said anti-TCRβV antibody molecules are disclosed in Table 29.

The various TCRβV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference. For example, TCRβ V6-5 is represented in approximately 3-6% healthy donors.

The representation of various TCRβV subfamilies and/or subfamily members can also be different in cancer cells. For example, TCRβV is present in about 3-6% of tumor infiltrating T cells irrespective of tumor type (see Li B. et al., Nature Genetics, 2016, vol: 48(7):725-32 the entire contents of which are hereby incorporated by references). Li et al., also disclose that TCRβ V6-5 is present at a high frequency in tumor cells.

### Anti-TCRβ V6 Antibodies

In one aspect, provided herein is an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ 3 V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments. TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments. TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 1043, or a sequence having 85%, 90%, 95%, 99% or more identity thereof. In some embodiments, TCRβ V6-5*01 comprises the amino acid sequence of SEQ ID NO: 1044, or an amino acid sequence having 85%, 90%, 95%, 99% or more identity thereof.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule. e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in Table 21, or encoded by a nucleotide sequence in Table 21, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 1231 or 1233.

-Consensus VH
SEQ ID NO: 1231
QVQLVQSGAEVKKPGSSVKVSCKASGH/T/G/YD/T/SFH/R/D/K/
TL/D/K/T/NW/F/T/I/Y/GYIHWVRQAPGQGLEWMGR/WV/I/FF/
S/YA/PGSGN/ST/V/Y/IK/RYNEKFKGRVTITADTSTSTAYMELSSL
RSEDTAVYYCAG/VSY/IYSY/AD/GVLDYWGQGTTVTVSS
-Consensus VH
SEQ ID NO: 1233
QVQLVQSGAEVKKPGSSVKVSCKASGX1X2FX3X4X5YIHWVRQAPGQG
LEWMGX6X7X8X9GSGX10X11X12YNEKFKGRVTITADTSTSTAYMEL
SSLRSEDTAVYYCAX13SX14YSX15X16VLDYWGQGTTVTVSS,

where-in: X1 is H or T or G or Y; X2 is D or T or S; X3 is H or R or D or K or T; X4 is L or D or K or T or N; X5 is W or F or T or I or Y or G: X6 is R or W; X7 is V or I or F; X8 is F or S or Y; X9 is A or P; X10 is N or S; X11 is T or V or Y or n; X12 is K or R; X13 is G or V; X14 is Y or I; X15 is Y or A; and X16 is D or G.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 1230 or 1232.

-Consensus VL
SEQ ID NO: 1230
DIQMTQSPSFLSASVGDRVTITCKASQNVG/E/A/DN/DR/KVAWY/
HQQKPGKAPKALIYSSSHRYK/SGVPSRFSGSGSGTEFTLTISSLQPED
FATYFCQQFKSYPLTFGQGTKLEIK
-Consensus VL
SEQ ID NO: 1232
DIQMTQSPSFLSASVGDRVTITCKASQNVX1X2X3VAWX4QQKPGKAPK
ALIYSSSHRYX5GVPSRFSGSGSGTEFTLTISSLQPEDFATYFCQQFKS
YPLTFGQGTKLEIK,

wherein X1 is G, E, A or D, X2 is N or D; X3 is R or K; X4 is Y or H; and X5 is K or S.

In some embodiments, the anti-TCRβV antibody molecule. e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgGI, e.g., a human IgGI. In some embodiments, the heavy chain constant region comprises an amino sequence set forth in Table 23, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In some embodiments, the light chain constant region comprises an amino sequence set forth in Table 23, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 21, or encoded by a nucleotide sequence shown in Table 21. In some embodiments, one or more of the CDRs (or collectively all of the CDRs) have one, two, three, four, five, six or more changes, e.g., amino acid substitutions or deletions, relative to the amino acid sequence shown in Table 21, or encoded by a nucleotide sequence shown in Table 21.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 21, or encoded by a nucleotide sequence shown in Table 21. In some embodiments, one or more of the CDRs (or collectively all of the CDRs) have one, two, three, four, five, six or more changes, e.g., amino acid substitutions or deletions, relative to the amino acid sequence shown in Table 21, or encoded by a nucleotide sequence shown in Table 21.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 21, or encoded by a nucleotide sequence shown in Table 21. In some embodiments, one or more of the CDRs (or collectively all of the CDRs) have one, two, three, four, five, six or more changes, e.g., amino acid substitutions or deletions, relative to the amino acid sequence shown in Table 21, or encoded by a nucleotide sequence shown in Table 21.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCR4V antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al (e.g., at least one, two, or three CDRs according to the Kabat definition as set out in Table 21) from a heavy chain variable region of an antibody described herein, e.g., an antibody chosen from any one of A-H.1 to A-H.85, e.g., A-H.1, A-H.2 or A-H.68, or an antibody described in Table 21, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 21.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 21) from a light chain variable region of an antibody described herein, e.g., an antibody chosen from any one of A-H.1 to A-H.85, e.g., A-H.1, A-H.2 or A-H.68, or an antibody described in Table 21, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 21.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 21) from the heavy and light chain variable regions of an antibody described herein, e.g., an antibody chosen from any one of A-H. 1 to A-H.85, e.g., A-H.1, A-H.2 or A-H.68, or an antibody described in Table 21, or encoded by a nucleotide sequence in Table 21; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions. e.g., conservative substitutions) relative to one, two, three, four, five, or six CDRs according to Kabat et al. shown in Table 21.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 21) from the heavy and light chain variable regions of an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 21. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 21) from a heavy chain variable region of an antibody described herein, e.g., an antibody chosen from any one of A-H.1 to A-H.85, e.g., A-H.1, A-H.2 or A-H.68, or as described in Table 21, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 21.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 21) from a light chain variable region of an antibody described herein, e.g., an antibody chosen from any one of A-H.1 to A-H.85, e.g., A-H.1, A-H.2 or A-H.68, or an antibody described in Table 21, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 21.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 21) from the heavy and light chain variable regions of an antibody described herein, e.g., an antibody chosen from any one of A-H.1 to A-H.85, e.g., A-H.1, A-H.2 or A-H.68, or an antibody described in Table 21, or encoded by the nucleotide sequence in Table 21; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 21.

In some embodiments, the anti-TCR4V antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 21) from the heavy and light chain variable regions of an antibody described herein, e.g., an antibody chosen from any one of A-H.1 to A-H.85, e.g., A-H.1, A-H.2 or A-H.68, or an antibody described in Table 21, or encoded by a nucleotide sequence in Table 21; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 21. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in Table 21.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in Table 21 is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 21. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in Table 21.

In some embodiments, e.g., an embodiment comprising a variable region, a CDR (e.g., a combined CDR, Chothia CDR or Kabat CDR), or other sequence referred to herein, e.g., in Table 21, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 1002, SEQ ID NO: 1010 or SEQ ID NO: 1011, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1001 or SEQ ID NO: 1009.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 1002, and a HC CDR1, HC CDR2, and HC CDR3 of SEQ ID NO: 1001.

In some embodiments the anti-TCRβ V antibody molecule, e.g., anti-TCRβ V6 (e.g., anti-TCRβ V6-5*01) antibody molecule comprises a LC CDR1, LC CDR2, and LC CDR3 of SEQ ID NO: 1010, and a HC CDR1, HC CDR2, and HC CDR3 of SEQ ID NO: 1009.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 1011, and a HC CDR1, HC CDR2, and HC CDR3 of SEQ ID NO: 1009.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 1006, a LC CDR2 amino acid sequence of SEQ ID NO: 1007, or a LC CDR3 amino acid sequence of SEQ ID NO: 1008; and/or (ii) a HC CDR1 amino acid sequence of SEQ ID NO: 1003, a HC CDR2 amino acid sequence of SEQ ID NO: 1004, or a HC CDR3 amino acid sequence of SEQ ID NO: 1005.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 1006, a LC CDR2 amino acid sequence of SEQ ID NO: 1007, or a LC CDR3 amino acid sequence of SEQ ID NO: 1008; and/or (ii) a heavy chain variable region (VH) comprising a HC CDR1 amino acid sequence of SEQ ID NO: 1003, a HC CDR2 amino acid sequence of SEQ ID NO: 1004, or a HC CDR3 amino acid sequence of SEQ ID NO: 1005.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 1006, a LC CDR2 amino acid sequence of SEQ ID NO: 1007, or a LC CDR3 amino acid sequence of SEQ ID NO: 1008; and/or (ii) a HC CDR1 amino acid sequence of SEQ ID NO: 1045, a HC CDR2 amino acid sequence of SEQ ID NO: 1004, or a HC CDR3 amino acid sequence of SEQ ID NO: 1005.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 1006, a LC CDR2 amino acid sequence of SEQ ID NO: 1007, or a LC CDR3 amino acid sequence of SEQ ID NO: 1008; and/or (ii) a heavy chain variable region (VH) comprising a HC CDR1 amino acid sequence of SEQ ID NO: 1045, a HC CDR2 amino acid sequence of SEQ ID NO: 1004, or a HC CDR3 amino acid sequence of SEQ ID NO: 1005.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 1006, a LC CDR2 amino acid sequence of SEQ ID NO: 1007, or a LC CDR3 amino acid sequence of SEQ ID NO: 1008; and/or (ii) a HC CDR1 amino acid sequence of SEQ ID NO: 1015, a HC CDR2 amino acid sequence of SEQ ID NO: 1016, or a HC CDR3 amino acid sequence of SEQ ID NO: 1005.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 1006, a LC CDR2 amino acid sequence of SEQ ID NO: 1007, or a LC CDR3 amino acid sequence of SEQ ID NO: 1008; and/or (ii) a heavy chain variable region (VH) comprising a HC CDR1 amino acid sequence of SEQ ID NO: 1015, a HC CDR2 amino acid sequence of SEQ ID NO: 1016, or a HC CDR3 amino acid sequence of SEQ ID NO: 1005.

In some embodiments, the anti-TCRβV antibody molecule, e.g., anti-TCRβ V6 (e.g., anti-TCRβ V6-5*01) antibody molecule comprises a VH and/or a VL of an antibody described in Table 21, or a sequence with at least 80%, 85%, 90%, 95%, %%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 21, or a sequence with at least 80%, 85%, 90%, 95%, 96%, 97%, 98%, 99% or more identity thereto.

In some embodiments, an anti-TCRVb antibody as described herein has an antigen binding domain having a VL having a consensus sequence of SEQ ID NO: 1230, wherein position 30 is G, E, A or D; position 31 is N or D; position 32 is R or K; position 36 is Y or H; and/or position 56 is K or S.

In some embodiments, an anti-TCRVb antibody as described herein has an antigen binding domain having a VH having a consensus sequence of SEQ ID NO: 1231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

Exemplary anti-TCRβ V6 antibodies are provided in Table 21. In some embodiments, the anti-TCRβV antibody comprises one or more (e.g., all three) of a LC CDR1, LC CDR2, and LC CDR3 provided in Table 21, and/or one or more (e.g., all three) of a HC CDR1, HC CDR2, and HC CDR3 provided in Table 21, or a sequence with at least 95% sequence identity thereto. In some embodiments, antibody comprises a variable heavy chain (VH) and/or a variable light chain (VL) provided in Table 21, or a sequence with at least 95% sequence identity thereto.

In some embodiments, the anti-TCRβ V6 antibody molecule comprises a VH and/or a VL of an antibody described in Table 21, or a sequence with at least 80%, 85%, 90%, 95%, %%, 97%, 98%, 99% or more identity thereto.

In some embodiments, the anti-TCRβ V6 antibody molecule comprises a VH and a VL of an antibody described in Table 21, or a sequence with at least 80%, 85%, 90%, 95%, 96%, 97%, 98%, 99% or more identity thereto.

### Anti-TCRβ V4 Antibodies

In one aspect, provided herein is an anti-TCRβV antibody molecule that binds to a human TCRβ V4 subfamily member. In some embodiments, the TCRβ V4 subfamily comprises: TCRβ V4-1*01, TCRβ V4-1*02, TCRβ V4-2*01, TCRβ V4-2*02, TCRβ V4-3*01, TCRβ V4-3*02, TCRβ V4-3*03, or TCRβ V4-3*04, or a variant thereof.

Exemplary anti-TCRβ V4 antibodies are provided in Table 30. In some embodiments, the anti-TCRβ V4 is antibody L, e.g., humanized antibody L (antibody L-H), as provided in Table 31. In some embodiments, antibody L comprises one or more (e.g., three) light chain CDRs and/or one or more (e.g., three) heavy chain CDRs provided in Table 30, or a sequence with at least 95% sequence identity thereto. In some embodiments, antibody L comprises a variable heavy chain (VH) and/or a variable light chain (VL) provided in Table 30, or a sequence with at least 95% sequence identity thereto.

In some embodiments, the anti-TCRβ V4 antibody molecule comprises a VH and/or a VL of an antibody described in Table 30, or a sequence with at least 80%, 85%, 90%, 95%, 96%, 97%, 98%, 99% or more identity thereto.

In some embodiments, the anti-TCRβ V4 antibody molecule comprises a VH and a VL of an antibody described in Table 30, or a sequence with at least 80%, 85%, 90%, 95%, 96%, 97%, 98%, 99% or more identity thereto.

### Anti-TCRαV Antibodies

### Human T Cell Receptor (TCR) Complex

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRαV chains can be further classified into subfamilies (TRAV1-10, 12-14, 16-27, 29, 30, 34-36 an 38-41). Despite their high structural and functional homology, the amino acid sequence homology in the TRAV genes is very low. Nevertheless. TCRs formed between alpha and beta chains of highly diverse sequences show a remarkable structural and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G, and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor alpha variable chain” or “TCRαV,” or “TRAV,” refers to an extracellular region of the T cell receptor alpha chain which can comprise a portion of the antigen recognition domain of the T cell receptor. The term TCRαV includes isoforms, mammalian, e.g., human TCRαV, species homologs of human and analogs comprising at least one common epitope with TCRαV. Human TCRαV comprises a gene family comprising subfamilies including, but not limited to: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, or a TCRα V41 subfamily, as well as family members of said subfamilies, and variants thereof (e.g., a structural or functional variant thereof).

In some embodiments, the TCRα V1 subfamily comprises: TCRαV1-1 or TCRαV1-2, or a variant thereof.

In some embodiments, the TCRα V1 subfamily comprises: TCRαV17-1 or a variant thereof.

In some embodiments, the anti-TCRαV antibody molecule is a full antibody or fragment thereof (e.g., a Fab, F(ab′)2, Fv, single domain antibody, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgGI or IgG2 (e.g., human IgGI, or IgG2). In some embodiments, the heavy chain constant region is human IgG1. In some embodiments, the Fc region comprises a Fc region variant, e.g., as described herein.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 1212 or 1214; or positions 135 (M to Y), 137 (S to T), 139 (T to E), 316 (H to K) and 317 (N to F) of SEQ ID NOs: 1215, 1216, 1217 or 1218), e.g., relative to human IgGI.

The various TCRαV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference.

In some embodiments, the anti-TCRαV antibody molecule is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule is isolated or recombinant.

In some embodiments, the anti-TCRαV antibody molecule comprises a heavy chain constant region for an IgG4, e.g., a human IgG4. In still another embodiment, the anti-TCRαV antibody molecule includes a heavy chain constant region for an IgGI, e.g., a human IgGI. In some embodiments, the heavy chain constant region comprises an amino sequence set forth in Table 23, or a sequence substantially identical (e.g., at least 80%, 85%, 90%, 92%, 95%, 97%, 98%, 99% or higher identical) thereto.

In some embodiments, the anti-TCRαV antibody molecule includes a kappa light chain constant region, e.g., a human kappa light chain constant region. In some embodiments, the light chain constant region comprises an amino sequence set forth in Table 23, or a sequence substantially identical (e.g., at least 80%, 85%, 90%, 92%, 95%, 97%, 98%, 99% or higher identical) thereto.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule, is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule, is isolated or recombinant.

In some embodiments, the anti-TCRαV antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule comprises a light chain variable domain comprising: (a) a framework region 1 (FR1) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRαV antibody molecule is a full antibody or fragment thereof (e.g., a Fab, F(ab′)2, Fv, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgGI or IgG2 (e.g., human IgGI, or IgG2). In some embodiments, the heavy chain constant region is human IgGI.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 1212 or 1214; or positions 135 (M to Y), 137 (S to T), 139 (T to E), 316 (H to K) and 317 (N to F) of SEQ ID NOs: 1215, 1216, 1217 or 1218).

### Anti-TCRα V1 Antibodies

In one aspect, provided herein is an anti-TCRαV antibody molecule that binds to a human TCRα V1 subfamily member. In some embodiments, the TCRα V1 subfamily comprises: TCRα V1-1*01, TCRα V1-1*02, TCRα V1-2*01, or TCRα V1-2*02, or a variant thereof.

Exemplary anti-TCRα V1 antibodies are provided in Table 22. In some embodiments, the anti-TCRα V1 is antibody L, e.g., humanized antibody L (antibody L-H), as provided in Table 31. In some embodiments, antibody L comprises one or more (e.g., three) light chain CDRs and/or one or more (e.g., three) heavy chain CDRs provided in Table 22, or a sequence with at least 95% sequence identity thereto. In some embodiments, antibody L comprises a variable heavy chain (VH) and/or a variable light chain (VL) provided in Table 22, or a sequence with at least 95% sequence identity thereto.

In some embodiments, the anti-TCRα V1 antibody molecule comprises a VH and/or a VL of an antibody described in Table 22, or a sequence with at least 80%, 85%, 90%, 95%, 96%, 97%, 98%, 99% or more identity thereto.

### Anti-TCRα V17 Antibodies

In one aspect, provided herein is an anti-TCRαV antibody molecule that binds to a human TCRα V17 subfamily member. In some embodiments, the TCRα V17 subfamily comprises. TCRα V17*01, or a variant thereof.

### Antibody-like Frameworks or Scaffolds

A wide variety of antibody/immunoglobulin frameworks or scaffolds can be employed in the anti-TCRvb or anti-TCRvα antibody molecules as described herein or multifunctional formats thereof so long as the resulting polypeptide includes at least one binding region which specifically binds to the target antigen. e.g., a TCRvb or TCRva, a tumor antigen, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-TCRvb or anti-TCRva antibody molecules as described herein or multifunctional formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRvb, TCRva. or a tumor antigen). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

Fibronectin scaffolds are typically based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-TCRvb antibody molecules as described herein or multifunctional formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Anti-TCRVβ Antibody or Anti-TCRVα Effector Function and Fc Variants

In some embodiments, an anti-TCRVβ or anti-TCRVα antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-TCRVβ or anti-TCRvα antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVβ or anti-TCRvα antibody comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVα or anti-TCRVα antibody comprising a variant Fc region has reduced affinity to a human Fc receptor, e.g., FcγR I, FcγR II and/or FcγR III. In some embodiments, the anti-TCRVβ or anti-TCRVα antibody comprising a variant Fc region comprises a human IgGI region or a human IgG4 region.

In some embodiments, an anti-TCRVβ or anti-TCRVα antibody comprising a variant Fc region activates and/or expands T cells, e.g., as described herein. In some embodiments, an anti-TCRVβ or anti-TCRvα antibody comprising a variant Fc region has a cytokine profile described herein, e.g., a cytokine profile that differs from a cytokine profile of a T cell engager that binds to a receptor or molecule other than a TCRβV region (“a non-TCRβV-binding T cell engager”) or a TCRαV region (“a non-TCR(V-binding T cell engager”). In some embodiments, the non-TCRβV-binding or non-TCRαV-binding T cell engager comprises an antibody that binds to a CD3 molecule (e.g., CD3 epsilon (CD3e) molecule); or a TCR alpha (TCRα) molecule.

Exemplary Fc region variants are provided in Table 31 and also disclosed in Saunders O, (2019) Frontiers in Immunology: vol 10, article1296, the entire contents of which is hereby incorporated by reference.

In some embodiments, an anti-TCRVβ or anti-TCRVα antibody as described herein comprises any one or all, or any combination of Fc region variants disclosed in Table 31.

In some embodiments, an anti-TCRVβ or anti-TCRVα antibody as described herein comprises any one or all, or any combination of Fc region variants, e.g., mutations, disclosed in Table 31. In some embodiments, an anti-TCRVβ or anti-TCRVα antibody as described herein comprise an Asn297Ala (N297A) mutation. In some embodiments, an anti-TCRVβ antibody or anti-TCRVα as described herein comprise a Leu234Ala/Leu235Ala (LALA) mutation.

### Multifunctional Molecules

As used herein, a “multifunctional” or a “multispecific” molecule refers to molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multispecific molecule is a multispecific antibody molecule. e.g., a bispecific antibody molecule. In some embodiments, the multispecific molecule includes an anti-TCRVb antibody or anti-TCRVα molecule as described herein.

The present disclosure provides a multispecific molecule comprises (a) first domain that binds to a first target molecule, wherein the first target molecule is a T cell receptor alpha variable chain 1-2 (TRAV1-2), a T cell receptor beta variable chain 6-2 (TRBV6-2), a T cell receptor beta variable chain 4-1 (TRBV4-1), or a T cell receptor alpha variable chain 17 (TRAV17); and (b) a second domain that binds to a second target molecule, wherein the second domain comprises (i) a cytokine or a functional fragment or variant thereof, or (ii) an antigen binding domain.

In some embodiments, the multispecific molecule is bound to a T cell. In some embodiments, the T cell binds to CD1b or CD1c presented antigens.

In some embodiments, the multispecific molecule is an agonist of TRAV1-2, TRBV6-2, TRBV4-1 or TRAV17.

In some embodiments, the multispecific molecule is an antagonist of TRAV1-2, TRBV6-2, TRBV4-1 or TRAV17.

In some embodiments, first domain binds to TRAV1-2. In some embodiments, the TRAV1-2 is TRAV1-2*01, TRAV1-2*02, or TRAV1-3*03. In some embodiments, first domain binds to TRBV6-2. In some embodiments, the TRBV6-2 is TRBV6-2*01. In some embodiments, first domain binds to TRBV4-L. In some embodiments, the TRBV4-1 is TRBV4-1*01. In some embodiments, first domain binds to TRAV17. In some embodiments, the TRAV17 is TRAV17*01.

In some embodiments, the second domain comprises a cytokine.

In some embodiments, the cytokine is selected from the group consisting of interleukin-2 (IL-2) or a fragment or a functional fragment or a functional variant thereof, interleukin-7 (IL-7) or a fragment or a functional fragment or a functional variant thereof, interleukin-12 (IL-12) or a fragment or a functional fragment or a functional variant thereof, interleukin-15 (IL-15) or a fragment or a functional fragment or a functional variant thereof, interleukin-18 (IL-18) or a fragment or a functional fragment or a functional variant thereof, interleukin-21 (IL-21) or a fragment or a functional fragment or a functional variant thereof, or interferon gamma or a fragment or a functional fragment or a functional variant thereof, or a combination thereof.

In some embodiments, the cytokine comprises interleukin-2 (IL-2) or a fragment thereof. In some embodiments, the cytokine is interleukin-2 (IL-2) or a fragment thereof. In some embodiments, the cytokine comprises a sequence having at least 75%, 80%, 85%, 90%, 95%, 99%, 99.5%, 99.9%, or 100% sequence identity to the sequence of SEQ ID NO: 1268. In some embodiments, the cytokine comprises the sequence of SEQ ID NO: 1268. In some embodiments, the sequence of the cytokine is a sequence having at least 75%, 80%, 85%, 90%, 95%, 99%, 99.5%, 99.9%, or 100% sequence identity to the sequence of SEQ ID NO: 1268. In some embodiments, the sequence of the cytokine is the sequence of SEQ ID NO: 1268.

In some embodiments, the variant of the cytokine comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the cytokine is an IL-2 variant comprising a mutation. In some embodiments, the mutation is an insertion mutation, a deletion mutation, or a substitution mutation. In some embodiments, the mutation is the substitution mutation. In some embodiments, the variant is an IL-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 1242. In some embodiments, the variant comprises the sequence of SEQ ID NO: 1242. In some embodiments, the sequence of the variant is a sequence having at least 75%, 80%, 85%, 90%, 95%, 99%, 99.5%, 99.9%, or 100% sequence identity to the sequence of SEQ ID NO: 1242. In some embodiments, the sequence of the variant is the sequence of SEQ ID NO: 1242.

In some embodiments, the cytokine comprises interleukin-7 (IL-7) or a fragment thereof. In some embodiments, the cytokine is interleukin-7 (IL-7) or a fragment thereof. In some embodiments, the cytokine comprises a sequence having at least 75%, 80%, 85%, 90%, 95%, 99%, 99.5%, 99.9%, or 100% sequence identity to the sequence of SEQ ID NO: 1273. In some embodiments, the cytokine comprises the sequence of SEQ ID NO: 1273. In some embodiments, the sequence of the cytokine is a sequence having at least 75%, 80%, 85%, 90%, 95%, 99%, 99.5%, 99.9%, or 100% sequence identity to the sequence of SEQ ID NO: 1273. In some embodiments, the sequence of the cytokine is the sequence of SEQ ID NO: 1273.

In some embodiments, the cytokine comprises interleukin-12 (IL-12) or a fragment thereof. In some embodiments, the cytokine is interleukin-12 (IL-12) or a fragment thereof. In some embodiments, the cytokine comprises a sequence having at least 75%, 80%, 85%, 90%, 95%, 99%, 99.5%, 99.9%, or 100% sequence identity to the sequence of SEQ ID NO: 1276. In some embodiments, the cytokine comprises the sequence of SEQ ID NO: 1276. In some embodiments, the sequence of the cytokine is a sequence having at least 75%, 80%, 85%, 90%, 95%, 99%, 99.5%, 99.9%, or 100% sequence identity to the sequence of SEQ ID NO: 1276. In some embodiments, the sequence of the cytokine is the sequence of SEQ ID NO: 1276.

In some embodiments, the cytokine comprises interleukin-15 (IL-15) or a fragment thereof. In some embodiments, the cytokine is interleukin-15 (IL-15) or a fragment thereof. In some embodiments, the cytokine comprises a sequence having at least 75%, 80%, 85%, 90%, 95%, 99%, 99.5%, 99.9%, or 100% sequence identity to the sequence of SEQ ID NO: 1297. In some embodiments, the cytokine comprises the sequence of SEQ ID NO: 1297. In some embodiments, the sequence of the cytokine is a sequence having at least 75%, 80%, 85%, 90%, 95%, 99%, 99.5%, 99.9%, or 100% sequence identity to the sequence of SEQ ID NO: 1297. In some embodiments, the sequence of the cytokine is the sequence of SEQ ID NO: 1297.

In some embodiments, the cytokine comprises interleukin-18 (IL-18) or a fragment thereof. In some embodiments, the cytokine is interleukin-18 (IL-18) or a fragment thereof. In some embodiments, the cytokine comprises a sequence having at least 75%, 80%, 85%, 90%, 95%, 99%, 99.5%, 99.9%, or 100% sequence identity to the sequence of SEQ ID NO: 1285. In some embodiments, the cytokine comprises the sequence of SEQ ID NO: 1285. In some embodiments, the sequence of the cytokine is a sequence having at least 75%, 80%, 85%, 90%, 95%, 99%, 99.5%, 99.9%, or 10% sequence identity to the sequence of SEQ ID NO: 1285. In some embodiments, the sequence of the cytokine is the sequence of SEQ ID NO: 1285.

In some embodiments, the cytokine comprises interleukin-21 (IL-21) or a fragment thereof. In some embodiments, the cytokine is interleukin-21 (IL-21) or a fragment thereof. In some embodiments, the cytokine comprises a sequence having at least 75%, 80%, 85%, 90%, 95%, 99%, 99.5%, 99.9%, or 100% sequence identity to the sequence of SEQ ID NO: 1286. In some embodiments, the cytokine comprises the sequence of SEQ ID NO: 1286. In some embodiments, the sequence of the cytokine is a sequence having at least 75%, 80%, 85%, 90%, 95%, 99%, 99.5%, 99.9%, or 100% sequence identity to the sequence of SEQ ID NO: 1286. In some embodiments, the sequence of the cytokine is the sequence of SEQ ID NO: 1286.

In some embodiments, multispecific molecule further comprises a third domain comprising an antigen binding domain.

In some embodiments, the antigen binding domain of the third domain binds to a tumor associated antigen (TAA). In some embodiments, the TAA is a tumor antigen, a stromal antigen, or a hematological antigen. In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu. Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1. MUM2, MUM3, NA88-1, NPM, OA1, OGT, RCC, RUI1, RUI2, SAGE, TRG, TRP1, TSTA, Folate receptor alpha. L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the second domain comprises an antigen binding domain.

In some embodiments, the multispecific molecule comprises a single TRAV1-2, TRBV6-2, TRBV4-1 or TRAV17 binding moiety. In some embodiments, the antigen binding domain of the second domain binds to TRAV1-2, TRBV6-2, TRBV4-1 or TRAV17.

In some embodiments, the first domain and the antigen binding domain of the second domain bind to: TRAV1-2 and TRAV1-2, respectively; TRAV1-2 and TRBV6-2, respectively; TRAV1-2 and TRBV4-1, respectively; TRAV1-2 and TRAV17, respectively; TRBV6-2 and TRAV1-2, respectively; TRBV6-2 and TRBV6-2, respectively; TRBV6-2 and TRBV4-1, respectively TRBV6-2 and TRAV17, respectively; TRBV4-1 and TRAV1-2, respectively; TRBV4-1 and TRBV6-2, respectively; TRBV4-1 and TRBV4-1, respectively; TRBV4-1 and TRAV17, respectively; TRAV17 and TRAV1-2, respectively; TRAV17 and TRBV6-2, respectively; TRAV17 and TRBV4-1, respectively; TRAV17 and TRAV17, respectively.

In some embodiments, the antigen binding domain of the second domain binds to a tumor associated antigen (TAA). In some embodiments, the TAA is a tumor antigen, a stromal antigen, or a hematological antigen. In some embodiments, the TAA is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2. Cyclin-BI, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1. EphA3, Epidermal growth factor receptor (EGFR), MART-2, MART-1, MUC1, MUC2, MUM1. MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the multispecific molecule further comprises a third domain comprising a cytokine.

In some embodiments, the multispecific molecule comprises a first polypeptide and a second polypeptide, wherein the first polypeptide and the second polypeptide are non-contiguous, wherein (i) the first polypeptide comprises a first portion of a dimerization module linked to (A) the first domain that binds to TRAV1-2, TRBV6-2, TRBV4-1 or TRAV17, wherein the first domain that binds to TRAV1-2, TRBV6-2, TRBV4-1 or TRAV17 comprises a single chain variable fragment (scFv), or a single domain antibody (sdAb), or (B) a first portion of the first domain that binds to TRAV1-2, TRBV6-2, TRBV4-1 or TRAV17, wherein the first portion of the first domain that binds to TRAV1-2, TRBV6-2, TRBV4-1 or TRAV17 comprises a heavy chain variable domain (VH), wherein when the first polypeptide comprises the first portion of the first domain that binds to TRAV1-2, TRBV6-2, TRBV4-1 or TRAV17, the multispecific molecule further comprises a third polypeptide comprising a second portion of the first domain that binds to TRAV1-2, TRBV6-2, TRBV4-1 or TRAV17, wherein the second portion of the first domain that binds to TRAV1-2, TRBV6-2, TRBV4-1 or TRAV17 comprises a light chain variable domain (VL), wherein the third polypeptide is non-contiguous with the first polypeptide and the second polypeptide; and (ii) the second polypeptide comprises a second portion of the dimerization module; wherein (a) the cytokine or functional fragment or variant thereof is covalently linked to the first polypeptide, the second polypeptide, or the third polypeptide, or (b) the antigen binding domain is covalently linked to the first polypeptide, the second polypeptide, or the third polypeptide.

In some embodiments, the first polypeptide comprises the first portion of the first domain that binds to TRAV1-2, TRBV6-2, TRBV4-1 or TRAV17, and wherein (i) the first portion of the first domain that binds to TRAV1-2, TRBV6-2, TRBV4-1 or TRAV17 is linked to the N-terminus of the first polypeptide comprising the first portion of the dimerization module; and (ii) the second polypeptide is linked to the C-terminus of a cytokine or a functional fragment or variant thereof.

In some embodiments, the multispecific molecule comprises a first polypeptide and a second polypeptide, wherein the first polypeptide and the second polypeptide are non-contiguous, wherein the first polypeptide comprises each of the following linked together: (i) a first portion of a dimerization module; and (ii) (A) a third domain comprising an antigen binding domain, wherein the third domain comprises a single chain variable fragment (scFv), or a single domain antibody (sdAb), or (B) a first portion of the third domain, wherein the first portion of the third domain comprises a heavy chain variable domain (VH), wherein when the first polypeptide comprises the first portion of the third domain, the multispecific molecule further comprises a third polypeptide comprising a second portion of the third domain, wherein the second portion of the third domain comprises a light chain variable domain (VL), wherein the third polypeptide is non-contiguous with the first polypeptide and the second polypeptide; and wherein the second polypeptide comprises each of the following linked together: (i) a second portion of the dimerization module; (ii) the second domain, wherein the second domain comprises a cytokine: (iii) (A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: (i) a first portion of a dimerization module; (ii) (A) the second domain comprising an antigen binding domain, wherein the second domain comprises a single chain variable fragment (scFv), or a single domain antibody (sdAb), or (B) a first portion of the second domain, wherein the first portion of the second domain comprises a heavy chain variable domain (VH), wherein when the first polypeptide comprises the first portion of the second domain, the multispecific molecule further comprises a third polypeptide comprising a second portion of the second domain, wherein the second portion of the second domain comprises a light chain variable domain (VL), wherein the third polypeptide is non-contiguous with the first polypeptide and the second polypeptide; and wherein the second polypeptide comprises each of the following linked together: (i) a second portion of the dimerization module; and (ii) (A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide.

In some embodiments, the multispecific molecule comprises a first polypeptide and a second polypeptide, wherein the first polypeptide and the second polypeptide are non-contiguous, and wherein the first polypeptide comprises each of the following linked together: (i) a first portion of a dimerization module; (ii) (A) the first domain, wherein the first domain comprises a single chain variable fragment (scFv), or a single domain antibody (sdAb), or (B) a first portion of the first domain, wherein the first portion of the first domain comprises a heavy chain variable domain (VH), wherein when the first polypeptide comprises the first portion of the first domain, the multispecific molecule further comprises a third polypeptide comprising a second portion of the first domain, wherein the second portion of the first domain comprises a light chain variable domain (VL), wherein the third polypeptide is non-contiguous with the first polypeptide and the second polypeptide; and (iii) (A) the second domain, wherein the second domain comprises the cytokine or a functional fragment or variant thereof, or a single chain variable fragment (scFv), or a single domain antibody (sdAb), or (B) a first portion of the second domain, wherein the second domain comprises the antigen binding domain, wherein the first portion of the second domain comprises a heavy chain variable domain (VH), wherein when the first polypeptide comprises the first portion of the second domain, the multispecific molecule further comprises a fourth polypeptide comprising a second portion of the second domain, wherein the second portion of the second domain comprises a light chain variable domain (VL), wherein the fourth polypeptide is non-contiguous with the first polypeptide and the second polypeptide, and wherein when the multispecific molecule comprises the third polypeptide the fourth polypeptide is non-contiguous with the third polypeptide; wherein the second polypeptide comprises each of the following linked together: (i) a second portion of the dimerization module; (ii) (A) an additional first domain, wherein the additional first domain comprises a single chain variable fragment (scFv), or a single domain antibody (sdAb), or (B) a first portion of the additional first domain, wherein the first portion of the additional first domain comprises a heavy chain variable domain (VH), wherein when the second polypeptide comprises the first portion of the additional first domain, the multispecific molecule further comprises a fifth polypeptide comprising a second portion of the additional first domain, wherein the second portion of the additional first domain comprises a light chain variable domain (VL), wherein the fifth polypeptide is non-contiguous with the first polypeptide and the second polypeptide, and wherein when the multispecific molecule comprises the third polypeptide and/or the fourth polypeptide the fifth polypeptide is non-contiguous with the third polypeptide and the fourth polypeptide; and (iii) (A) an additional second domain, wherein the second domain comprises the cytokine or a functional fragment or variant thereof, or a single chain variable fragment (scFv), or a single domain antibody (sdAb), or (B) a first portion of the additional second domain, wherein the additional second domain comprises an additional antigen binding domain, wherein the first portion of the additional second domain comprises a heavy chain variable domain (VH), wherein when the second polypeptide comprises the first portion of the additional second domain, the multispecific molecule further comprises a sixth polypeptide comprising a second portion of the additional second domain, wherein the second portion of the additional second domain comprises a light chain variable domain (VL), wherein the sixth polypeptide is non-contiguous with the first polypeptide and the second polypeptide, and wherein when the multispecific molecule comprises the third polypeptide, the fourth polypeptide, and/or the fifth polypeptide, the sixth polypeptide is non-contiguous with the third polypeptide, the fourth polypeptide, and the fifth polypeptide.

In some embodiments, the first portion of the dimerization molecule comprises a first immunoglobulin constant regions (Fc regions) and the second portion of the dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgGI Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold. 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold. 650 fold, 700 fold, 750 fold, 800 fold, 850 fold, 900 fold, 950 fold. 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold. 75 fold, 80 fold, 85 fold, 90 fold, 95 fold, 100 fold, 150 fold, 200 fold, 250 fold, 300 fold, 250 fold, 400 fold, 450 fold. 500 fold, 550 fold, 600 fold, 650 fold, 700 fold, 750 fold, 800 fold, 850 fold, 900 fold, 950 fold, 1000 fold, 2000 fold, 3000 fold, 4000 fold. 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an amino acid substitution listed in Table 31.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 1040, SEQ ID NO: 1042, SEQ ID NO: 1204, SEQ ID NO: 1205, SEQ ID NO: 1041, SEQ ID NO: 1206, or SEQ ID NO: 1207. In some embodiments, the first Fc region, the second Fc region, or a combination thereof comprises the sequence of SEQ ID NO: 1040, SEQ ID NO: 1042, SEQ ID NO: 1204, SEQ ID NO: 1205, SEQ ID NO: 1041, SEQ ID NO: 1206, or SEQ ID NO: 1207.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 1040, SEQ ID NO: 1042, SEQ ID NO: 1204, SEQ ID NO: 1205, SEQ ID NO: 1041, SEQ ID NO: 1206, or SEQ ID NO: 1207. In some embodiments, the sequence of the first Fc region, the second Fc region, or a combination thereof is the sequence of SEQ ID NO: 1040, SEQ ID NO: 1042, SEQ ID NO: 1204, SEQ ID NO: 1205, SEQ ID NO: 1041, SEQ ID NO: 1206, or SEQ ID NO: 1207.

In some embodiments, the first TCRβV-binding moiety, the second TCRβV-binding moiety, or a combination thereof binds to one or more of a TCRβV subfamily selected from the group consisting of: (i) TCRβ V4 subfamily comprising one or more selected from TCRβ V4-1*01, TCRβ V4-1*02, TCRβ V4-2*01, and TCRβ V4-2*02; (ii) the TCRβ V6 subfamily comprising one or more selected from TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01, and TCRβ V6-1*01.

In some embodiments, the first TCRβV-binding moiety and the second TCRβV-binding moiety are same. In some embodiments, the first TCRβV-binding moiety and the second TCRβV-binding moiety are different.

In some embodiments, the first TCRβV-binding moiety and the second TCR4V-binding moiety binds: (i) one or more of a TCRβ V4 subfamily member and one or more of a TCRβ V6 subfamily member, respectively; (ii) one or more of a TCRβ V6 subfamily member and one or more of a TCRβ V4 subfamily member, respectively.

In some embodiments, the first domain that binds to TRAV1-2, TRBV6-2, TRBV4-1 or TRAV17, a second domain that binds to TRAV1-2, TRBV6-2, TRBV4-1 or TRAV17, or any combination thereof comprises a Fab, a F(ab′)2, an Fv, a single chain Fv (scFv), a single domain antibody, a diabody (dAb), a camelid antibody, and a combination thereof.

In some embodiments, the first domain that binds to TRAV1-2, TRBV6-2, TRBV4-1 or TRAV17, a second domain that binds to TRAV1-2, TRBV6-2, TRBV4-1 or TRAV17, or any combination thereof comprises a Fab or an scFv.

In some embodiments, the first domain that binds to TRAV1-2, TRBV6-2, TRBV4-1 or TRAV17, the second domain that binds to TRAV1-2, TRBV6-2, TRBV4-1 or TRAV17, or a combination thereof comprises: (i) a HC CDR1, a HC CDR2 and a HC CDR3 of an amino acid sequence having at least 75%, 80%, 85%, 90%, 95%, 99%, 99.5%. 99.9%, or 100% sequence identity to any one of the CDR1, CDR2, and CDR3 sequences listed in Table 21, Table 22, Table 32, or Table 33, respectively; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 the sequences listed in Table 21, Table 22, Table 32, or Table 33, respectively; or (iii) a combination thereof. In some embodiments, the first domain that binds to TRAV1-2, TRBV6-2, TRBV4-1 or TRAV17, the second domain that binds to TRAV1-2, TRBV6-2, TRBV4-1 or TRAV17, or a combination thereof comprises: (i) a HC CDR1, a HC CDR2 and a HC CDR3 having any one of the CDR1, CDR2, and CDR3 sequences listed in Table 21, Table 22, Table 32, or Table 33, respectively; (ii) a LC CDR1, a LC CDR2, and a LC CDR3 having any one of the CDR1, CDR2, and CDR3 the sequences listed in Table 21, Table 22, Table 32, or Table 33, respectively; or (iii) a combination thereof.

In some embodiments, the first domain that binds to TRAV1-2, TRBV6-2, TRBV4-1 or TRAV17, the second domain that binds to TRAV1-2, TRBV6-2, TRBV4-1 or TRAV17, or a combination thereof comprises: (i) a VH of an amino acid sequence having at least 75%, 80%, 85%, 90%, 95%, 99%, 99.5%, 99.9%, or 100/6 sequence identity to any one of the VH sequences listed in Table 21, Table 22, Table 32, or Table 33; (ii) a VL of an amino acid sequence having at least 75%, 80%, 85%, 90%, 95%, 99%, 99.5%, 99.9%, or 100% sequence identity to any one of the VL sequences listed in Table 21, Table 22, Table 32, or Table 33; or (iii) a combination thereof. In some embodiments, the first domain that binds to TRAV1-2, TRBV6-2, TRBV4-1 or TRAV17, the second domain that binds to TRAV1-2, TRBV6-2, TRBV4-1 or TRAV17, or a combination thereof comprises: (i) a VH having any one of the VH sequences listed in Table 21, Table 22, Table 32, or Table 33; (ii) a VL having any one of the VL sequences listed in Table 21, Table 22, Table 32, or Table 33, respectively; or (iii) a combination thereof.

In some embodiments, the composition described herein comprises a second multispecific molecule, wherein the second multispecific molecule comprises a fourth domain that binds to TRBV6-2, TRBV4-1, or TRAV17.

In some embodiments, the second multispecific molecule comprises a fifth domain, and wherein the fifth domain comprises (i) a cytokine or a functional fragment or variant thereof, or (ii) an antigen binding domain.

In some embodiments, the fifth domain comprises a cytokine.

In some embodiments, the fifth domain comprises an antigen binding domain.

In some embodiments, the second multispecific molecule comprises a sixth domain comprising an antigen binding domain. In some embodiments, the antigen binding domain of the sixth domain binds to TRAV1-2, TRBV6-2, TRBV4-1 or TRAV17.

In some embodiments, the antigen binding domain of the sixth domain binds to a TAA.

In some embodiments, the second multispecific molecule comprises a sixth domain comprising a cytokine. In some embodiments, the antigen binding domain of the fourth domain binds to TRAV1-2, TRBV6-2, TRBV4-1 or TRAV17. In some embodiments, the antigen binding domain of the fourth domain binds to a TAA.

In some embodiments, the first domain, the second domain, or a combination thereof comprises a heavy chain constant region having a sequence having at least 75%, 80%, 85%, 90%, 95%, 99%, 99.5%, 99.9%, or 100% sequence identity to any one of the sequences listed in Table 23 or a combination thereof. In some embodiments, the first domain, the second domain, or a combination thereof comprises a heavy chain constant region having any one of the sequences listed in Table 23 or a combination thereof. In some embodiments, the first domain, the second domain, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 23 or a combination thereof. In some embodiments, the first domain, the second domain, or a combination thereof comprises a heavy chain constant region having any one of the heavy chain constant region sequences listed in Table 23 or a combination thereof. In some embodiments, the first domain, the second domain, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 1073. In some embodiments, the heavy chain constant region of the IgM comprises the sequence of SEQ ID NO: 1073. In some embodiments, the sequence of the heavy chain constant region of the IgM is the sequence of SEQ ID NO: 1073.

In some embodiments, the first domain, the second domain, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 1076. In some embodiments, the heavy chain constant region of the IgJ comprises the sequence of SEQ ID NO: 1076. In some embodiments, the sequence of the heavy chain constant region of the IgJ is the sequence of SEQ ID NO: 1076.

In some embodiments, the first domain, the second domain, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 1074. In some embodiments, the heavy chain constant region of the IgGA1 comprises the sequence of SEQ ID NO: 1074. In some embodiments, the sequence of the heavy chain constant region of the IgGA1 is the sequence of SEQ ID NO: 1074.

In some embodiments, the first domain, the second domain, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5%, 99.9%, or 100% sequence identity to the sequence of SEQ ID NO: 1075. In some embodiments, the heavy chain constant region of the IgGA2 comprises the sequence of SEQ ID NO: 1075. In some embodiments, the sequence of the heavy chain constant region of the IgGA2 is the sequence of SEQ ID NO: 1075.

In some embodiments, the first domain, the second domain, or a combination thereof comprises a heavy chain constant region of an IgGI or a fragment thereof. In some embodiments, the heavy chain constant region of the IgGI comprises a sequence having at least 75%, 80%, 85%, 90%, 95%, 99%, 99.5%, 99.9%, or 100% sequence identity to the sequence of SEQ ID NO: 1041. In some embodiments, the heavy chain constant region of the IgGI comprises the sequence of SEQ ID NO: 1041. In some embodiments, the sequence of the heavy chain constant region of the IgGI is the sequence of SEQ ID NO: 1041. In some embodiments, the heavy chain constant region of the IgGI comprises a sequence having at least 75%, 80%, 85%, 90%, 95%, 99%, 99.5%, 99.9%, or 100% sequence identity to the sequence of SEQ ID NO: 1204. In some embodiments, the heavy chain constant region of the IgGI comprises the sequence of SEQ ID NO: 1204. In some embodiments, the sequence of the heavy chain constant region of the IgGI is the sequence of SEQ ID NO: 1204.

In some embodiments, the first domain, the second domain, or a combination thereof comprises a light chain constant region having a sequence having at least 75%, 80%, 85%, 90%, 95%, 99%, 99.5%, 99.9%, or 100% sequence identity to any one of the sequences listed in Table 23 or a combination thereof. In some embodiments, the first domain, the second domain, or a combination thereof comprises a light chain constant region having any one of the sequences listed in Table 23 or a combination thereof. In some embodiments, the first domain, the second domain, or a combination thereof comprises a light chain constant region having any one of the light chain constant region sequences listed in Table 23 or a combination thereof.

In the first domain, the second domain, or a combination thereof comprises a light chain constant region of a kappa chain or a fragment thereof. In some embodiments, the light chain constant region of a kappa chain comprises a light chain constant region sequence listed in Table 3.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 1039 or SEQ ID NO: 1203. In some embodiments, the light chain constant region of a kappa chain comprises the sequence of SEQ ID NO: 1039 or SEQ ID NO: 1203. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 1039 or SEQ ID NO: 1203. In some embodiments, the sequence of the light chain constant region of a kappa chain is the sequence of SEQ ID NO: 1039 or SEQ ID NO: 1203.

### Cytokine Molecules

In some embodiments, the multifunctional molecule includes a cytokine molecule. As used herein, a “cytokine molecule” or a “cytokine” as interchangeably used herein, refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits activity of a naturally-occurring cytokine. In some embodiments the cytokine molecule is chosen from interleukin-2 (IL-2), interleukin-7 (IL-7), interleukin-12 (IL-12), interleukin-10 (IL-10), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Provided herein are, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF. IL-la, IL-1p, IL-2, IL-3, IL-4, IL-5, IL-6, IL-7, IL-8, IL-10, IL-12, IL-21, IFN-α, IFN-0, IFN-γ, MIP-la, MIP-1f, TGF-0, TNF-α, and TNFβ. In some embodiments the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In some embodiments the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In some embodiments, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment, the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In some embodiments, the mutant IL-2 cytokine of the multispecific or multifunctional polypeptide according to the invention comprises amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In some embodiments the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In some embodiments the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In some embodiments said additional amino acid mutation is the amino acid substitution C125A.

In a specific embodiment the IL-2 cytokine of the multispecific or multifunctional polypeptide comprises the polypeptide sequence of SEQ ID NO: 1242

[APTSSSTKKTQLQLEHLLLDLQMILNGINNYKNPKLTRMLTFKFYMPK
KATELKHLQCLEEELKPLEEVLNLAQSKNFHLRPRDLISNINVIVLELK
GSETTFMCEYADETATIVEFLNRWITFAQSIISTLT].

In another specific embodiment the IL-2 cytokine of the multispecific or multifunctional polypeptide comprises the polypeptide sequence of SEQ ID NO: 1280

[APASSSTKKTQLQLEHLLLDLQMILNGINNYKNPKLTRMLTAKFAMPK
KATELKHLQCLEEELKPLEEVLNGAQSKNFHLRPRDLISNINVIVLELK
GSETTFMCEYADETATIVEFLNRWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 1276

[IWELKKDVYVVELDWYPDAPGEMVVLTCDTPEEDGITWTLDQSSEVLG
SGKTLTIQVKEFGDAGQYTCHKGGEVLSHSLLLLHKKEDGIWSTDILKD
QKEPKNKTFLRCEAKNYSGRFTCWWLTTISTDLTFSVKSSRGSSDPQGV
TCGAATLSAERVRGDNKEYEYSVECQEDSACPAAEESLPIEVMVDAVHK
LKYENYTSSFFIRDIIKPDPPKNLQLKPLKNSRQVEVSWEYPDTWSTPH
SYFSLTFCVQVQGKSKREKKDRVFTDKTSATVICRKNASISVRAQDRYY
SSSWSEWASVPCSGGGGSGGGGSGGGGSRNLPVATPDPGMFPCLHHSQN
LLRAVSNMLQKARQTLEFYPCTSEEIDHEDITKDKTSTVEACLPLELTK
NESCLNSRETSFITNGSCLASRKTSFMMALCLSSIYEDLKMYQVEFKTM
NAKLLMDPKRQIFLDQNMLAVIDELMQALNFNSETVPQKSSLEEPDFYK
TKIKLCILLHAFRIRAVTIDRVMSYLNAS].

In some embodiments, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 1281

[SPGQGTQSENSCTHFPGNLPNMLRDLRDAFSRVKTFFQMKDQLDNLLL
KESLLEDFKGYLGCQALSEMIQFYLEEVMPQAENQDPDIKAHVNSLGEN
LKTLRLRLRRCHRFLPCENKSKAVEQVKNAFNKLQEKGIYKAMSEFDIF
INYIEAYMTMKIRNGGGGSGGGGSGGGGSGGGGSSPGQGTQSENSCTHF
PGNLPNMLRDLRDAFSRVKTFFQMKDQLDNLLLKESLLEDFKGYLGCQA
LSEMIQFYLEEVMPQAENQDPDIKAHVNSLGENLKTLRLRLRRCHRFLP
CENKSKAVEQVKNAFNKLQEKGIYKAMSEFDIFINYIEAYMTMKIRN].

In another specific embodiment the IL-10 cytokine is a monomeric IL-10 cytokine. In a more specific embodiment the monomeric IL-10 cytokine comprises the polypeptide sequence of SEQ ID NO: 1282

[SPGQGTQSENSCTHFPGNLPNMLRDLRDAFSRVKTFFQMKDQLDNLLL
KESLLEDFKGYLGCQALSEMIQFYLEEVMPQAENQDPDIKAHVNSLGEN
LKTLRLRLRRCHRFLPCENGGGSGGKSKAVEQVKNAFNKLQEKGIYKAM
SEFDIFINYIEAYMTMKIRN].

In some embodiments, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In some embodiments the mutant IL-15 cytokine of the multispecific or multifunctional polypeptide according to the invention comprises amino acid mutation that reduces or abolishes the affinity of the mutant IL-15 cytokine to the alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In some embodiments the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In some embodiments the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 1312 [NWVNVISDLKKIEDLIQSMHIDATLYTESDVHPSCKVTAMKCFLLELQVISLASGDASIH DTVENLIILANNSLSSNGAVTESGCKECEELEEKNIKEFLQSFVHIVQMFINTS]. In some embodiments, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In some embodiments, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In some embodiments the cytokine, particularly a single-chain cytokine, of the multispecific or multifunctional polypeptide is IFNγ. In a specific embodiment, the IFN-7 cytokine can elicit one or more of the cellular responses selected from the group of: increased macrophage activity, increased expression of MHC molecules, and increased NK cell activity. In some embodiments the cytokine, particularly a single-chain cytokine, of the multispecific or multifunctional polypeptide is IL-7. In a specific embodiment, the IL-7 cytokine can elicit proliferation of T and/or B lymphocytes. In some embodiments, the cytokine, particularly a single-chain cytokine, of the multispecific or multifunctional polypeptide is IL-8. In a specific embodiment, the IL-8 cytokine can elicit chemotaxis in neutrophils. In some embodiments, the cytokine, particularly a single-chain cytokine, of the multispecific or multifunctional polypeptide, is MIP-la. In a specific embodiment, the MIP-la cytokine can elicit chemotaxis in monocytes and T lymphocyte cells. In some embodiments, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In some embodiments, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In some embodiments, the multispecific or multifunctional polypeptide of the invention binds to a cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules as described herein include a cytokine molecule. In embodiments, the cytokine molecule includes a full length, a fragment or a variant of a cytokine: a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In some embodiments, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 1238), a fragment thereof, or an amino acid sequence substantially identical thereto (e.g., 95% to 99.9% identical thereto, or having amino acid alteration, but not more than five, ten or fifteen alterations (e.g., substitutions, deletions, or insertions, e.g., conservative substitutions) to the amino acid sequence of SEQ ID NO: 1238.

In some embodiments, the cytokine molecule comprises a receptor dimerizing domain, e.g., an IL15Ralpha dimerizing domain. In some embodiments, the IL15Ralpha dimerizing domain comprises the amino acid sequence: MAPRRARGCRTLGLPALLLLLLLRPPATRGITCPPPMSVEHADIWVKSYSLYSRERYICN SGFKRKAGTSSLTECVL (SEQ ID NO: 1283), a fragment thereof, or an amino acid sequence substantially identical thereto (e.g., 95% to 99.9% identical thereto, or having amino acid alteration, but not more than five, ten or fifteen alterations (e.g., substitutions, deletions, or insertions, e.g., conservative substitutions) to the amino acid sequence of SEQ ID NO: 1283. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 1284). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 1268), a fragment thereof, or an amino acid sequence substantially identical thereto (e.g., 95% to 99.9% identical thereto, or having amino acid alteration, but not more than five, ten or fifteen alterations (e.g., substitutions, deletions, or insertions, e.g., conservative substitutions) to the amino acid sequence of SEQ ID NO: 1268).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGM AVTISVKCEKISTLSCENKIISFKEMNPPDNIKDTKSDIIFFQRSVPGHDNKMQFESSSYEG YFLACEKERDLFKLILKKEDELGDRSIMFTVQNED (SEQ ID NO: 1285), a fragment thereof, or an amino acid sequence substantially identical thereto (e.g., 95% to 99.9% identical thereto, or having amino acid alteration, but not more than five, ten or fifteen alterations (e.g., substitutions, deletions, or insertions, e.g., conservative substitutions) to the amino acid sequence of SEQ ID NO: 1285).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSA NTGNNERIINVSIKKLKRKPPSTNAGRRQKHRLTCPSCDSYEKKPPKEFLERFKSLLQKMI HQHLSSRTHGSEDS (SEQ ID NO: 1286), a fragment thereof, or an amino acid sequence substantially identical thereto (e.g., 95% to 99.9% identical thereto, or having amino acid alteration, but not more than five, ten or fifteen alterations (e.g., substitutions, deletions, or insertions, e.g., conservative substitutions) to the amino acid sequence of SEQ ID NO: 1286).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFK NFKDDQSIQKSVETIKEDMNVKFFNSNKKKRDDFEKLTNYSVTDLNVQRKAIHELIQVM AELSPAAKTGKRKRSQMLFRG (SEQ ID NO: 1287), a fragment thereof, or an amino acid sequence substantially identical thereto (e.g., 95% to 99.9% identical thereto, or having amino acid alteration, but not more than five, ten or fifteen alterations (e.g., substitutions, deletions, or insertions, e.g., conservative substitutions) to the amino acid sequence of SEQ ID NO: 1287).

### Immune Cell Engagers

In some embodiments, the multifunctional molecule further includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

The immune cell engagers, e.g., first and/or second immune cell engager, of the multispecific or multifunctional molecules as described herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager. NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### T Cell Engagers

Provided herein are, inter alia, multispecific (e.g., bi-, tri-, quad-specific) or multifunctional molecules that are engineered to further contain one or more T cell engager that mediate binding to and/or activation of a T cell. In some embodiments, the T cell engager is an antigen binding domain that binds to, e.g., activates TCRβ, e.g., a TCRβV region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226.

In some embodiments, the multispecific or multifunctional molecules are engineered to activate a CD1b or CD1c restricted T cell. In some embodiments, the T cell binds to CD1b or CD1c presented antigens. In some embodiments, the T cell is a GEM T cell or a LDN5-like T cells. LDN5-like T cells is a type of human T cell that have specificity for CD1b or Cd1c and mycobacterial lipid glucose-6-O-monomycolate (GMM), and the expression of TRBV4-1 or TRAV17-utilizing TCRs. Germline-encoded mycolyl lipid-specific (GEM) T cells are a subset of human T cells that have specificity for CD1b or CD1c and GMM. They have preferential expression of TRAV1-2 and TRAJ9.

### Tumor-Targeting Moieties

In some embodiments, the multifunctional or multispecific (e.g., bi-, tri-, tetra-specific) molecules as described herein further include, e.g., are engineered to further contain, one or more tumor specific targeting moieties that direct the molecule to a tumor cell.

In certain embodiments, the multispecific molecules as described herein further include a tumor-targeting moiety. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules as described herein to a cancer (e.g., a cancer or tumor cells). In some embodiments, the cancer is chosen from a hematological cancer, a solid cancer, a metastatic cancer, or a combination thereof.

In some embodiments, the multispecific molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In some embodiments, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-BI, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, α actinin-4, TRP1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In other embodiments, the multispecific molecule, e.g., the tumor-targeting moiety, binds to a molecule, e.g., antigen, present on the surface of a hematological cancer, e.g., a leukemia or a lymphoma. In some embodiments, the hematological cancer is a B-cell or T cell malignancy. In some embodiments, the hematological cancer is chosen from one or more of a Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome (MDS), multiple myeloma, or acute lymphocytic leukemia. In embodiments, the cancer is other than acute myeloid leukemia (AML) or myelodysplastic syndrome (MDS). In embodiments, the hematological antigen is chosen from CD47, CD99, CD30, CD38, SLAMF7, or NY-ESO1. In some embodiments, the hematological antigen is chosen from is chosen from one or more of: BCMA, CD19, CD20, CD22, CD33, CD123, FcRH5, CLEC12, or CD179A.

In some embodiments, the tumor-associated antigen-binding moiety comprises an antibody or an antigen-binding moiety thereof that binds to CD19, CD123, CD22, CD30, CD171, CS-1, C-type lectin-like molecule-1, CD33, CISH, epidermal growth factor receptor variant III (EGFRvIII), ganglioside G2 (GD2), ganglioside GD3, TNF receptor family member B cell maturation (BCMA), Tn antigen, prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA). Epithelial cell adhesion molecule (EPCAM), B7H3 (CD276), KIT (CD117). Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit. Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasacchande portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR5 IE2), TCR Gamma Alternate Reading Frame Protein (TARP), Wilms tumor protein (WT1), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant prostein, survivin, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene). N-Acetyl glucosaminyl-transferase V (NA17), paired box protein Pax-3 (PAX3), Androgen receptor, Cyclin BI, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma. X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1) antigen.

### CD1b and CD1c Restricted T Cells

In some embodiments, the composition described herein can be used to expand or activate a population of T cells.

In some embodiments, the T cell binds to Cd1b or Cd1c presented antigens.

CD1 molecules are MHC class I-related molecules specialized in presenting lipid antigens, and humans can express five CD1 genes: CD1a, CD1b and CD1c (group 1); CD1d (group2); and CD1e (group 3). CD1-restricted T cells can recognize foreign lipids from pathogenic bacteria and can react against specific cell-endogenous lipids.

A T cell that binds to CD1b or CD1c can express or utilize TCR variable chains that are different from other types of T cells.

In some embodiments a T cell that binds to CD1b or CD1c can be referred to as a “CD1b-restricted T cell”, a “CD1c-restricted T cell,” or a “GMM specific T cell.”

In some embodiments, the TCR variable chains are TRAV1-2, TRBV6-2, TRBV4-1 or TRAV17.

Cd1b- or CD1c-restricted T cells can selectively bind to a mycobacterial glycolipid antigen, mycobacterial lipid glucose-6-O-monomycolate (GMM). They utilize GMM-specific TCRs. GMM-specific TCRs may be used to isolate natural CD1b, CD1c, and other glycolipid-reactive T cell repertoire in humans.

In some embodiments, a CD1b or Cd1c-restricted T cell is CD3+, CD4+, CD8+, TRBV4-1+, TRAV17+, TRBV6-2+, TRAV1-2+, or a combination thereof. In some embodiments, a CD1b or Cd1c-restricted T cell CD4+/CD8−, CD4−/CD8+, CD4−/CD8−, CD69+, or a combination thereof.

In some embodiments, a Cd1b- or CD1c-restricted T cell selectively binding to GMM includes a germline-encoded mycolyl lipid specific (GEM) T cells and a LDN5-like T cell. In some embodiments, a GEM T cell is CD4+CD8−. In some embodiments, a LDN5-like T cell is CD4+, CD8+, CD4+CD8+, or CD4−CD8−.

Non-limiting examples of TCRs used by GEM T cells include TRAV1-2 and TRBV6-2.

Non-limiting examples of TCRs used by LDN5-like cells include TRAV17 and TRBV 4-1.

In some embodiments, a cytokine profile can be used to measure the activation or stimulation of a Cd1b or CD1c-restricted T cell (e.g., GEM T cell, LDN5-like T cell). Any suitable cytokine assay, e.g., Luminex multiple cytokine assay can be used to measure cytokine released by an activated or stimulated CD1b or CD1c-restricted T cell compared to an inactivated or unstimulated CD1b or CD1c-restricted T cell. Non-limiting examples of cytokines released by an activated or stimulated CD1b or CD1c-restricted T cell include IL-1RA, IL-6, IL-13, IL-17, IL-25, CCL17, IFN-γ, or TNF.

In some embodiments, an activated or stimulated CD1b or CD1c-restricted T cell can release lower levels of cytokines compared to an inactivated or unstimulated CD1b or CD1c-restricted T cell. Non-limiting examples of cytokines that are produced at a lower level in an activated or stimulated CD1b or CD1c-restricted T cell compared to an inactivated or unstimulated CD1b or CD1c-restricted T cell include IL-4 IL-5, IL-10, IL-21, or IL-22.

In some embodiments, an activated or stimulated CD1b or CD1c-restricted T cell have differential production of cytokines characteristic for the helper T cell subsets Th1, Th17, and Th2, the acute phase and antiviral cytokines IL-6, TNF, LT-α, IFN-α, and IFN-β, the chemokines IP10 (CXCL10), XCL-1, MIP1β (CCL4), 1-309 (CCL1), and TARC (CCL17), and the negatively regulating mediators IL-10 and the IL-1 receptor antagonist compared to an inactivated or unstimulated CD1b or CD1c-restricted T cell.

### Antibody Molecules

In some embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In some embodiments, an antibody molecule is a monospecific antibody molecule and binds a single epitope. E.g., a monospecific antibody molecule having a plurality of immunoglobulin variable domain sequences, each of which binds the same epitope.

In some embodiments, an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or a tetraspecific antibody molecule.

In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv or a Fab, or fragment thereof, have binding specificity for a first epitope and a scFv or a Fab, or fragment thereof, have binding specificity for a second epitope.

In some embodiments, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423426; and Huston et al. (1988) Proc. Natl. Acad. Sci. LISA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of; Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner et al. U.S. Pat. No. 5,223,409; Kang et al. International Publication No. WO 92/18619: Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047: Garrard et al. International Publication No. WO 92/09690; Ladner et al. International Publication No. WO 90/02809: Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896: Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci, USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric. CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly. e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see. e.g., LoBuglio et al., Hybridoma, 5:5117-5123 (1986)).

|Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043): Liu et al. (1987) PNAS 84:3439-3443; Liu et al., 1987, J Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g. rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgAQ1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g, altered affinity for an effector ligand, such as FcR on a cell, or the Cl component of complement can be produced by replacing amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### CDR-Grafted Scaffolds

In some embodiments, the antibody molecule is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds, and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type 111 repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes. e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581 A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US201503152% A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Multispecific Molecules

In some embodiments, the multispecific molecules as described herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of, Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgGI For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

In some embodiments, Fc contains exemplary Fc modifications listed in Table 31.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway. J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W): “Holes” or “cavities” are created by replacing a larger residue in a parental antibody with a smaller amino acid (e.g., Y407T, T366S, L368A and/or Y407V).

Exemplary “hole” heavy chains are provided in Table 33. In some embodiments, the “hole” heavy chain comprises one or more (e.g., three) heavy chain CDRs provided in Table 33, or a sequence with at least 95% sequence identity thereto.

Exemplary “hole” light chains are provided in Table 33. In some embodiments, the “hole” heavy chain comprises one or more (e.g., three) heavy chain CDRs provided in Table 33, or a sequence with at least 95% sequence identity thereto.

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary “knob” chains are provided in Table 33. In some embodiments, the “knob” chain comprises a sequence provided in Table 33, or a sequence with at least 95% sequence identity thereto.

Exemplary KiH mutations include S354C, T366W in the “knob” heavy chain and Y349C, T366S, L368A, Y407V in the “hole” heavy chain. Other exemplary KiH mutations are provided in Table 24, with additional optional stabilizing Fc cysteine mutations.

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1. US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgGI hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgGI antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CHI and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CHI) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CHI and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017 and designated publication number WO 2018/057955, incorporated herein by reference in its entirety.

In some embodiments, the multispecific molecule includes a multispecific antibody molecule, e.g., an antibody molecule comprising two binding specificities, e.g., a bispecific antibody molecule. The multispecific antibody molecule includes: a lambda light chain polypeptide 1 (LLCP1) specific for a first epitope; a heavy chain polypeptide 1 (HCP1) specific for the first epitope; a kappa light chain polypeptide 2 (KLCP2) specific for a second epitope; and a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In some embodiments, it comprises all or a fragment of a CH1 region. In some embodiments, an LLCP1 comprises LC-CDR1, LC-CDR2, LC-CDR3, FRI, FR2, FR3, FR4, and CHI, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In some embodiments, it comprises all or a fragment of a CHI region. In some embodiments, a KLCP2 comprises LC-CDR1, LC-CDR2, LC-CDR3, FRI, FR2, FR3, FR4, and CHI, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CHI region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments, in the multifunctional polypeptide molecule as described herein: LLCP1 has a higher affinity for HCP1 than for HCP2; and/or KLCP2 has a higher affinity for HCP2 than for HCP1.

In some embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments, in the multifunctional polypeptide molecule as described herein: the HCP1 has a greater affinity for HCP2, than for a second molecule of HCP1; and/or the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99, 99.5 or 99.9% of the multispecific antibody molecule molecules have a HCP1 complexed, or interfaced with, a HCP2.

In another aspect, described herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CHI, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CHI, a second heavy chain constant region (e.g., a second CH2, a second CH3, or both));
(iii) providing a lambda chain polypeptide (e.g., a lambda light variable region (VLAλ), a lambda light constant chain (VLλ), or both) that preferentially associates with the first heavy chain polypeptide (e.g., the first VH); and
(iv) providing a kappa chain polypeptide (e.g., a lambda light variable region (VLλ), a lambda light constant chain (VLλ), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments, (i)-(iv) (e.g., nucleic acid encoding (i)-(iv)) are introduced in a single cell, e.g., a single mammalian cell, e.g., a CHO cell. In some embodiments, (i)-(iv) are expressed in the cell. In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In some embodiments, the method further comprises purifying a cell-expressed antibody molecule, e.g., using a lambda—and/or—kappa-specific purification, e.g., affinity chromatography.

In some embodiments, the method further comprises evaluating the cell-expressed multispecific antibody molecule. For example, the purified cell-expressed multispecific antibody molecule can be analyzed by techniques known in the art, include mass spectrometry. In some embodiments, the purified cell-expressed antibody molecule is cleaved, e.g., digested with papain to yield the Fab moieties and evaluated using mass spectrometry.

In some embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CHI, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope:
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope; and
(iv) a kappa light chain polypeptide (KLCP2) (e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multispecific antibody molecule has a first binding specificity that includes a hybrid VLλ-CLλ heterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fc constant, CH2-CH3 domain (having a hole modification).

### Linkers

The multispecific or multifunctional molecule as described herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Ser. In some embodiments, the peptide linker is selected from GGGGS (SEQ ID NO: 1259); GGGGSGGGGS (SEQ ID NO: 1237); GGGGSGGGGSGGGGS (SEQ ID NO: 1258); DVPSGPGGGGGSGGGGS (SEQ ID NO: 1305); and GGGGSGGGGSGGGGGS (SEQ ID NO: 1306). In some embodiments, the peptide linker is a A(EAAAK)nA (SEQ ID NO: 1302) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 1260); AEAAAKEAAAKEAAAKAAA (SEQ ID NO: 1261); AEAAAKEAAAKEAAAKEAAAKAAA (SEQ ID NO: 1262); and AEAAAKEAAAKEAAAKEAAAKEAAAKAAA (SEQ ID NO: 1263).

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antibody molecules, e.g., anti-TCRβV antibody molecules, anti-TCRαV antibody molecules multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRαV antibody molecules, anti-TCRαV antibody molecules, or a multispecific or multifunctional molecule described herein. In some embodiments, the vectors comprise nucleic acid sequences encoding antibody molecules, e.g., anti-TCRβ V antibody molecules, anti-TCRαV antibody molecules, or multispecific or multifunctional molecule described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### CRS Grading

In some embodiments, CRS (Cytokine Release Syndrome) can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 25, Table 26, and Table 27. Unless otherwise specified, CRS as used herein refers to CRS according to the criteria of Table 26.

In some embodiments, CRS is graded according to Table 25.

The term “cytokine profile” as used herein, refers to the level and/or activity of on one or more cytokines or chemokines, e.g., as described herein. In some embodiments, a cytokine profile comprises the level and/or activity of a naturally occurring cytokine, a fragment or a functional fragment or a functional variant thereof. In some embodiments, a cytokine profile comprises the level and/or activity of one or more cytokines and/or one or more chemokines (e.g., as described herein). In some embodiments, a cytokine profile comprises the level and/or activity of a naturally occurring cytokine, a fragment or a functional fragment or a functional variant thereof. In some embodiments, a cytokine profile comprises the level and/or activity of a naturally occurring chemokine, a fragment or a functional fragment or a functional variant thereof. In some embodiments, a cytokine profile comprises the level and/or activity of one or more of: IL-2 (e.g., full length, a variant, or a fragment thereof); IL-1beta (e.g., full length, a variant, or a fragment thereof); IL-6 (e.g., full length, a variant, or a fragment thereof); TNFα (e.g., full length, a variant, or a fragment thereof); IFNgamma (e.g., full length, a variant, or a fragment thereof) IL-10 (e.g., full length, a variant, or a fragment thereof): IL-4 (e.g., full length, a variant, or a fragment thereof); TNF alpha (e.g., full length, a variant, or a fragment thereof); IL-12p70 (e.g., full length, a variant, or a fragment thereof); IL-13 (e.g., full length, a variant, or a fragment thereof); IL-8 (e.g., full length, a variant, or a fragment thereof); Eotaxin (e.g., full length, a variant, or a fragment thereof); Eotaxin-3 (e.g., full length, a variant, or a fragment thereof); IL-8 (HA) (e.g., full length, a variant, or a fragment thereof); IP-10 (e.g., full length, a variant, or a fragment thereof); MCP-1 (e.g., full length, a variant, or a fragment thereof); MCP-4 (e.g., full length, a variant, or a fragment thereof); MDC (e.g., full length, a variant, or a fragment thereof); MIP-la (e.g., full length, a variant, or a fragment thereof); MIP-1b (e.g., full length, a variant, or a fragment thereof); TARC (e.g., full length, a variant, or a fragment thereof); GM-CSF (e.g., full length, a variant, or a fragment thereof); IL-12 23p40 (e.g., full length, a variant, or a fragment thereof); IL-15 (e.g., full length, a variant, or a fragment thereof): IL-16 (e.g., full length, a variant, or a fragment thereof); IL-17a (e.g., full length, a variant, or a fragment thereof); IL-la (e.g., full length, a variant, or a fragment thereof); IL-5 (e.g., full length, a variant, or a fragment thereof); IL-7 (e.g., full length, a variant, or a fragment thereof); TNF-beta (e.g., full length, a variant, or a fragment thereof); or VEGF (e.g., full length, a variant, or a fragment thereof). In some embodiments, a cytokine profile includes secretion of one or more cytokines or chemokines. In some embodiments, a cytokine in a cytokine profile can be modulated, e.g., increased or decreased, by an anti-TCRβV antibody or an anti-TCRAV antibody molecule described herein. In some embodiments, the cytokine profile includes cytokines associated with a cytokine storm or cytokine release syndrome (CRS), e.g., IL-6, IL-1beta, TNFalpha and IL-10.

### Pharmaceutical Compositions

Described herein, in certain embodiments, is a pharmaceutical composition comprising the multifunctional polypeptide molecule as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multifunctional or multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific molecules or the compositions as described herein, or a pharmaceutically acceptable salt, solvate, hydrate or ester thereof. The pharmaceutical formulation comprising the multifunctional or multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate. 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific molecules as described herein. In some embodiments, the multifunctional or multispecific molecules or the compositions as described herein is administered in combination with another drug or therapeutic agent.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the multifunctional or multispecific molecules or the compositions as described herein may vary depending on cell type to which delivery of the multifunctional or multispecific molecules or the compositions is desired. The multifunctional or multispecific molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multifunctional or multispecific molecules or the compositions as described herein are administered with one or more agents capable of promoting penetration of the subject the multifunctional or multispecific molecules or the compositions as described herein across the blood-brain barrier by any method known in the art. For example, delivery of agents by administration of an adenovirus vector to motor neurons in muscle tissue is described in U.S. Pat. No. 6,632,427. “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the multifunctional or multispecific molecules or the compositions as described herein are linked or conjugated with agents that provide desirable pharmaceutical or pharmacodynamic properties. In some embodiments, the multifunctional or multispecific molecules or the compositions as described herein are coupled to a substance, known in the art to promote penetration or transport across the blood-brain barrier, e.g., an antibody to the transferrin receptor. In some embodiments, the multifunctional or multispecific molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Methods of Infection Treatment

Described herein, in certain embodiments, is a method of treating a condition or disease in a subject in need therefor comprising administering to the subject a therapeutically effective amount of the multifunctional polypeptide molecule as described herein, the nucleic acid molecules as described herein, the vector as described herein, the cell as described herein, the pharmaceutical composition as described herein, or a combination thereof, wherein the administering is effective to treat the condition or disease in the subject.

In some embodiments, the condition or disease is an infection. In some embodiments, the infection is a S. parathyphi infection, a Bacteroidetes infection, a Proteobacteria infection, a M. tuberculosis infection, a respiratory virus infection, a SARS-CoV-2 infection, yellow fever, influenza, a HIV infection, a Cytomegalovirus infection, and an Epstein-Barr Virus infection.

### Methods of Cancer Treatment

Described herein, in certain embodiments, is a method of treating a condition or disease in a subject in need therefor comprising administering to the subject a therapeutically effective amount of the multifunctional polypeptide molecule as described herein, the nucleic acid molecules as described herein, the vector as described herein, the cell as described herein, the pharmaceutical composition as described herein, or a combination thereof, wherein the administering is effective to treat the condition or disease in the subject.

In some embodiments, the condition or disease is cancer. In some embodiments, the cancer is a solid tumor, a hematological cancer, a metastatic cancer, a soft tissue tumor, or a combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and a combination thereof. In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and a combination thereof. In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and a combination thereof. In some embodiments, the T-cell lymphoma is peripheral T-cell lymphoma.

Non-limiting examples of cancers that can be treated with the compounds of the present disclosure include cancer cells from the bladder, blood, bone, bone marrow, brain, breast, colon, esophagus, gastrointestine, gum, head, kidney, liver, lung, nasopharynx, neck, ovary, prostate, skin, stomach, pancreas, prostate testis, tongue, cervix, or uterus. Non-limiting examples of cancer histological types include neoplasm, malignant; carcinoma; carcinoma, undifferentiated; giant and spindle cell carcinoma; small cell carcinoma; papillary carcinoma; squamous cell carcinoma: lymphoepithelial carcinoma: basal cell carcinoma; pilomatrix carcinoma; transitional cell carcinoma: papillary transitional cell carcinoma: adenocarcinoma; gastrinoma, malignant; cholangiocarcinoma; hepatocellular carcinoma: combined hepatocellular carcinoma and cholangiocarcinoma; trabecular adenocarcinoma; adenoid cystic carcinoma; adenocarcinoma in adenomatous polyp: adenocarcinoma, familial polyposis coli; solid carcinoma: carcinoid tumor, malignant; branchiolo-alveolar adenocarcinoma; papillary adenocarcinoma; chromophobe carcinoma; acidophil carcinoma; oxyphilic adenocarcinoma: basophil carcinoma: clear cell adenocarcinoma; granular cell carcinoma; follicular adenocarcinoma: papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma: ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma: medullary carcinoma; lobular carcinoma; inflammatory carcinoma; paget's disease, mammary; acinar cell carcinoma; adenosquamous carcinoma: adenocarcinoma w/squamous metaplasia; thymoma, malignant; ovarian stromal tumor, malignant; thecoma, malignant; granulosa cell tumor, malignant: androblastoma, malignant; sertoli cell carcinoma; leydig cell tumor, malignant; lipid cell tumor, malignant; paraganglioma, malignant: extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma: fibrosarcoma; fibrous histiocytoma, malignant; myxosarcoma; liposarcoma; leiomyosarcoma; rhabdomyosarcoma: embryonal rhabdomyosarcoma; alveolar rhabdomyosarcoma; stromal sarcoma; mixed tumor, malignant: mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant: struma ovarii, malignant: choriocarcinoma; mesonephroma, malignant; hemangiosarcoma; hemangioendothelioma, malignant; kaposi's sarcoma; hemangiopericytoma, malignant: lymphangiosarcoma; osteosarcoma; juxtacortical osteosarcoma: chondrosarcoma; chondroblastoma, malignant; mesenchymal chondrosarcoma; giant cell tumor of bone: ewing's sarcoma; odontogenic tumor, malignant; ameloblastic odontosarcoma; ameloblastoma, malignant; ameloblastic fibrosarcoma; pinealoma, malignant: chordoma; glioma, malignant; ependymoma; astrocytoma; protoplasmic astrocytoma; fibrillary astrocytoma: astroblastoma; glioblastoma; oligodendroglioma; oligodendroblastoma; primitive neuroectodermal: cerebellar sarcoma: ganglioneuroblastoma; neuroblastoma; retinoblastoma; olfactory neurogenic tumor; meningioma, malignant; neurofibrosarcoma: neurilemmoma, malignant; granular cell tumor, malignant: malignant lymphoma; hodgkin's disease; hodgkin's; paragranuloma; malignant lymphoma, small lymphocytic; malignant lymphoma, large cell, diffuse: malignant lymphoma, follicular; mycosis fungoides; other specified non-hodgkin's lymphomas; malignant histiocytosis; multiple myeloma; mast cell sarcoma: immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia: megakaryoblastic leukemia; myeloid sarcoma; and hairy cell leukemia. In some embodiments, the tumor may comprise an osteosarcoma, angiosarcoma, rhabdosarcoma, leiormyosarcoma, Ewing sarcoma, glioblastoma, neuroblastoma, or leukemia.

In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA, CD19, CD20, CD22, CD30, CD33, FcRH5, PDL1, CD47, CD117 (c-kit), gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, calcitonin, Cyclin-B1, Cyclin D1, DCP, 9D7, Ep-CAM, EphA3, Gastrin, HE4, Her2/neu, Telomerase, SAP-1, Survivin, NY-ESO-1/LAGE-1, PRAME, SSX-2, Melan-A/MART-1, 5-HIAA, Gp100/pmel17, Tyrosinase, TRP-1/-2, MCIR, s-catenin, BRCA1/2, CDK4, CML66, Fibronectin, p53, Ras, TGF-B receptor, AFP, ETA, MAGE, MUC-1, CA15-3, CA27.29, CA19-9, CA-125, BAGE, GAGE, NY-ESO-1, β-catenin, CDK4, CDC27, α actinin-4, TRP1/gp75, TRP2, gp100, lactate dehydrogenase, Melan-A/MART1, gangliosides, WT1, EphA3, Epidermal growth factor receptor (EGFR), MART-2, MART-1, MPO, MUC1, MUC2, MUM1, MUM2, MUM3, NA88-1, nuclear matrix protein 22, NPM, OA1, OGT, PAP, PD-L1, RCC, RUI1, RUI2, SAGE, TdT, TPMT, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

Methods described herein include treating a cancer in a subject by using multispecific or multifunctional molecules as described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

Described herein are methods of treating a subject having a cancer comprising acquiring a status of one or more TCRβV molecules in a subject. In some embodiments, a higher, e.g., increased, level or activity of one or more TCRβV molecules in a subject, e.g., in a sample from a subject, is indicative of a bias, e.g., a preferential expansion, e.g., clonal expansion, of T cells expressing said one or more TCRβV molecules in the subject.

Without wishing to be bound by theory, it is believed that a biased T cell population, e.g., a T cell population expressing a TCRβV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRβV molecules associated with the cancer. In some embodiments, the TCRβV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRβV molecules in a sample from the subject, comprising contacting said immune effector cell population with an anti-TCRβV antibody molecule as described herein, e.g., an anti-TCRβV antibody molecule that binds to the same TCRβV molecule that is higher, e.g., increased in the immune effector cell population in the sample from the subject. In some embodiments, contacting the population of immune effector cells (e.g., comprising T cells that express one or more TCRβV molecules) with an anti-TCRβV molecule results in expansion of the population of immune effector cells expressing one or more TCRβV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRβV molecules in a sample from the subject, and determining whether the one or more TCRβV molecules is higher, e.g., increased, in a sample from the subject compared to a reference value, wherein responsive to said determination, administering to the subject an effective amount of an anti-TCRβV antibody molecule, e.g., an agonistic anti-TCRβV antibody molecule, e.g., as described herein.

In some embodiments, the subject has B-CLL. In some embodiments, a subject having B-CLL has a higher, e.g., increased, level or activity of one or more TCRβV molecules, e.g., one or more TCRβV molecules comprising: (i) TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01.

In some embodiments, a subject having B-CLL has a higher, e.g., increased, level or activity of a TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRAV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, the subject has melanoma. In some embodiments, a subject having melanoma has a higher, e.g., increased, level or activity of one or more TCRAV molecules, e.g., one or more TCRβV molecules comprising the TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, acquiring a value for the status, e.g., presence, level and/or activity, of one or more TCRV molecules comprises acquiring a measure of the T cell receptor (TCR) repertoire of a sample. In some embodiments, the value comprises a measure of the clonotype of a population of T cells in the sample.

In some embodiments, a value for the status of one or more TCRβV molecules is obtained, e.g., measured, using an assay described in Wang C Y, et al., Int J Oncol. (2016) 48(6):2247-56, the entire contents of which are hereby incorporated by reference.

In some embodiments, a value for the status of one or more TCRAV molecules is obtained, e.g., measured, using flow cytometry.

Described herein are methods of treating a subject having a cancer comprising acquiring a status of one or more TCRAV molecules in a subject. In some embodiments, a higher, e.g., increased, level or activity of one or more TCRαV molecules in a subject, e.g., in a sample from a subject, is indicative of a bias, e.g., a preferential expansion, e.g., clonal expansion, of T cells expressing said one or more TCRαV molecules in the subject.

Without wishing to be bound by theory, it is believed that a biased T cell population, e.g., a T cell population expressing a TCRαV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRαV molecules associated with the cancer. In some embodiments, the TCRαV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRαV molecules in a sample from the subject, comprising contacting said immune effector cell population with an anti-TCRαV antibody molecule as described herein, e.g., an anti-TCRαV antibody molecule that binds to the same TCRαV molecule that is higher, e.g., increased in the immune effector cell population in the sample from the subject. In some embodiments, contacting the population of immune effector cells (e.g., comprising T cells that express one or more TCRAV molecules) with an anti-TCRαV molecule results in expansion of the population of immune effector cells expressing one or more TCRαV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRαV molecules in a sample from the subject, and determining whether the one or more TCRαV molecules is higher, e.g., increased, in a sample from the subject compared to a reference value, wherein responsive to said determination, administering to the subject an effective amount of an anti-TCRAV antibody molecule, e.g., an agonistic anti-TCRαV antibody molecule, e.g., as described herein.

Described herein are methods of treating a subject having a cancer comprising acquiring a status of one or more TCRAV molecules in a subject. In some embodiments, a higher, e.g., increased, level or activity of one or more TCRαV molecules in a subject, e.g., in a sample from a subject, is indicative of a bias, e.g., a preferential expansion, e.g., clonal expansion, of T cells expressing said one or more TCRAV molecules in the subject.

Without wishing to be bound by theory, it is believed that a biased T cell population, e.g., a T cell population expressing a TCRαV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRαV molecules associated with the cancer. In some embodiments, the TCRαV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRαV molecules in a sample from the subject, comprising contacting said immune effector cell population with an anti-TCRαV antibody molecule as described herein, e.g., an anti-TCRαV antibody molecule that binds to the same TCRβV molecule that is higher, e.g., increased in the immune effector cell population in the sample from the subject. In some embodiments, contacting the population of immune effector cells (e.g., comprising T cells that express one or more TCRαV molecules) with an anti-TCRαV molecule results in expansion of the population of immune effector cells expressing one or more TCRαV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRαV molecules in a sample from the subject, and determining whether the one or more TCRαV molecules is higher, e.g., increased, in a sample from the subject compared to a reference value, wherein responsive to said determination, administering to the subject an effective amount of an anti-TCRαV antibody molecule, e.g., an agonistic anti-TCRαV antibody molecule, e.g., as described herein.

### Combination Therapy

In some embodiments, the method as described herein further comprises administering a third therapeutic agent or therapy to the subject.

In some embodiments, the third therapeutic agent or therapy comprises a chemotherapeutic agent, a biologic agent, a hormonal therapy, radiation, or surgery.

In some embodiments, the third therapeutic agent or therapy is administered in combination with the first agent and second agent as described herein, the nucleic acid molecules as described herein, the vector as described herein, the cell as described herein, the pharmaceutical composition as described herein, sequentially, simultaneously, or concurrently.

The first agent and second agent as described herein can be used in combination with a third therapeutic agent or procedure.

In some embodiments, the first agent and second agent as described herein and the third therapeutic agent or procedure are administered/performed after a subject has been diagnosed with a cancer, e.g., before the cancer has been eliminated from the subject. In some embodiments, the multispecific or multifunctional molecules as described herein and the third therapeutic agent or procedure are administered/performed simultaneously or concurrently. For example, the delivery of one treatment is still occurring when the delivery of the third commences, e.g., there is an overlap in administration of the treatments. In other embodiments, the first agent and second agent as described herein and the third therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first, second, and second agent is more effective (e.g., leads to a greater reduction in symptoms and/or cancer cells) than the first, second, or third treatment alone. In some embodiments, the combination therapy permits use of a lower dose of the first, the second, or the third treatment compared to the dose of the first, the second, or the third treatment normally required to achieve similar effects when administered as a monotherapy. In some embodiments, the combination therapy has a partially additive effect, wholly additive effect, or greater than additive effect.

In some embodiments, the TCRβV antibody, multispecific or multifunctional molecule is administered in combination with a therapy, e.g., a cancer therapy (e.g., one or more of cancer agents, immunotherapy, photodynamic therapy (PDT), surgery and/or radiation). The terms “chemotherapeutic,” “chemotherapeutic agent,” and “cancer agent” are used interchangeably herein. The administration of the multispecific or multifunctional molecule and the therapy, e.g., the cancer therapy, can be sequential (with or without overlap) or simultaneous. Administration of the TCRβV antibody, multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

Methods described herein include treating a cancer in a subject by using the multispecific or multifunctional molecules as described herein, e.g., using a pharmaceutical composition as described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the cancer is a hematological cancer. In some embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm. Langerhans cell histiocytosis, multiple myeloma/plasma cell neoplasm, myelodysplastic syndrome, or myelodysplastic/myeloproliferative neoplasm.

In some embodiments, the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the subject has myelofibrosis. In some embodiments, the subject has a calreticulin mutation, e.g., a calreticulin mutation as described herein. In some embodiments, the subject does not have the JAK2-V617F mutation. In some embodiments, the subject has the JAK2-V617F mutation. In some embodiments, the subject has a MPL mutation. In some embodiments, the subject does not have a MPL mutation.

In some embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the cancer is acute lymphoblastic leukemia, acute lymphocytic leukemia, acute myelogenous leukemia, aplastic anemia, chronic myelogenous leukemia, desmoplastic small round cell tumor, Ewing's sarcoma, Hodgkin's disease, multiple myeloma, myelodysplasia, Non-Hodgkin's lymphoma, paroxysmal nocturnal hemoglobinuria, radiation poisoning, chronic lymphocytic leukemia, AL amyloidosis, essential thrombocytosis, polycythemia vera, severe aplastic anemia, neuroblastoma, breast tumors, ovarian tumors, renal cell carcinoma, autoimmune disorders, such as systemic sclerosis, osteopetrosis, inherited metabolic disorders, juvenile chronic arthritis, adrenoleukodystrophy, amegakaryocytic thrombocytopenia, sickle cell disease, severe congenital immunodeficiency, Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome, leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. In some embodiments, the cancer is a solid tumor. In some embodiments, the cancer is hematological.

In some embodiments, the combination therapy as described herein (or pharmaceutical composition as described herein) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g., Rosenberg et al., New Eng. J. of Med. 319:1676, 1988).

In some embodiments, the combination therapy as described herein or the pharmaceutical composition as described herein is administered to the subject parentally. In some embodiments, the cells are administered to the subject intravenously, subcutaneously, intratumorally, intranodally, intramuscularly, intradermally, or intraperitoneally. In some embodiments, the cells are administered, e.g., injected, directly into a tumor or lymph node. In some embodiments, the cells are administered as an infusion (e.g., as described in Rosenberg et al., New Eng. J. of Med. 319:1676, 1988) or an intravenous push. In some embodiments, the cells are administered as an injectable depot formulation.

In some embodiments, the subject is a mammal. In some embodiments, the subject is a human, monkey, pig, dog, cat, cow, sheep, goat, rabbit, rat, or mouse. In some embodiments, the subject is a human. In some embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In some embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Anti-Cancer Therapies

In other embodiments, the combination therapy as described herein is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®)), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®)), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATNOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate. TOPOSAR®, VEPESID®, ETOPOPHOS®), floxuridine (FUDR®), fludarabine (FLUDARA®), fluorouracil (cream) (CARAC™, EFUDEX®, FLUOROPLEX® t), gemcitabine (GEMZAR®), hydroxyurea (HYDREA®, DROXIA™, MYLOCEL™), idarubicin (IDAMYCIN®), ifosfamide (IFEX®), improsulfan, ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), piposulfan, procarbazine (MATULANE®), streptozocin (ZANOSAR®), temozolomide (TEMODAR®), teniposide (VM-26, VUMON®), TESPA (thiophosphoamide, thiotepa, TSPA. THIOPLEX®), topotecan (HYCAMTIN®), vinblastine (vinblastine sulfate, vincaleukoblastine, VLB, ALKABAN-AQ®, VELBAN®)), vinorelbine (vinorelbine tartrate, NAVELBINE®), and vorinostat (ZOLINZA®).

In another embodiment, the combination therapy as described herein is administered in conjunction with a biologic. Biologics useful in the treatment of cancers are known in the art and a binding molecule as described herein may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP: a nonsteroidal aromatase inhibitor which blocks aromatase, an enzyme needed to make estrogen); Aromasin® (exemestane, Pfizer Inc., New York, N.Y.: an irreversible, steroidal aromatase inactivator used in the treatment of breast cancer): FEMARA® (letrozole, Novartis Pharmaceuticals, East Hanover, N.J.: a nonsteroidal aromatase inhibitor approved by the FDA to treat breast cancer); and NOLVADEX® (tamoxifen. AstraZeneca Pharmaceuticals, LP; a nonsteroidal antiestrogen approved by the FDA to treat breast cancer). Other biologics with which the binding molecules as described herein may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride. Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.: a small molecule designed to target the human epidermal growth factor receptor 1 (HER1) pathway).

For the treatment of multiple myeloma, exemplary biologics include VELCADE® (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

Additional exemplary cancer therapeutic agents include, but are not limited to alkylating agents (e.g., thiotepa and cyclosphosphamide), alkyl sulfonates (e.g., busulfan, improsulfan, and piposulfan), aziridines (e.g., such as benzodopa, carboquone, meturedopa, and uredopa), ethylenimines and methylamelamines (e.g., altretamine, triethylenemelamine, trietylenephosphoramide, triethiylenethiophosphoramide, and trimethylolomelamine), acetogenins (e.g., bullatacin and bullatacinone), camptothecin (e.g., topotecan): bryostatin; callystatin, CC-1065 (e.g., adozelesin, carzelesin and bizelesin), cryptophycins (e.g., cryptophycin 1 and cryptophycin 8), dolastatin, duocarmycin (e.g., KW-2189 and CB1-TM1); eleutherobin, pancratistatin, sarcodictyin, spongistatin, nitrogen mustards (e.g., chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard) nitrosureas (e.g., carmustine, chlorozotocin, fotemustine, lomustine, nimustine, and ranimnustine), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (e.g., morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin, anti-metabolites (e.g., methotrexate and 5-fluorouracil (5-FU)), folic acid analogues (e.g., denopterin, pteropterin, and trimetrexate), purine analogs (e.g., fludarabine, 6-mercaptopurine, thiamiprine, and thioguanine) pyrimidine analogs (e.g., ancitabine, azacitidine, 6-azauridine, carmofur, cytarabine, dideoxyuridine, doxifluridine, enocitabine, and floxuridine); androgens (e.g., calusterone, dromostanolone propionate, epitiostanol, mepitiostane, and testolactone), anti-adrenals (e.g., as mitotane and trilostane), folic acid replenisher (e.g., frolinic acid; aceglatone; aldophosphamide glycoside), aminolevulinic acid, eniluracil, amsacrine, bestrabucil, bisantrene, edatrexate, defofamine, demecolcine, diaziquone, elformithine, elliptinium acetate, epothilone, etoglucid, gallium nitrate, hydroxyurea, lentinan, lonidainine, maytansinoids (e.g., such as maytansine and ansamitocins), mitoguazone, mitoxantrone, mopidanmol, nitraerine, pentostatin, phenamet, pirarubicin, losoxantrone, podophyllinic acid, 2-ethylhydrazide, procarbazine, PSKpolysaccharide complex, razoxane, rhizoxin, sizofiran, spirogermanium, tenuazonic acid, triaziquone, 2,2′,2″-trichlorotriethylamine, trichothecenes (e.g., T-2 toxin, verracurin A, roridin A and anguidine), urethan, vindesine, dacarbazine, mannomustine, mitobronitol, mitolactol, pipobroman, gacytosine, arabinoside (e.g., Ara-C), cyclophosphamide; toxoids (e.g., paclitaxel and docetaxel), gemcitabine, 6-thioguanine, mercaptopurine, platinum coordination complexes (cisplatin, oxaliplatin, and carboplatin), vinblastine, platinum, etoposide (e.g., VP-16), ifosfamide, mitoxantrone, vincristine, vinorelbine, novantrone, teniposide, edatrexate, daunomycin, aminopterin, xeloda, ibandronate, irinotecan (e.g., CPT-11), topoisomerase inhibitor, difluoromethylomithine (DMFO), retinoids (retinoic acid), capecitabine, carboplatin, procarbazine, and plicamycin.

In some embodiments, the combination therapy as described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299. JX-594 (thymidine kinase-deleted vaccinia virus plus GM-CSF), HPV-16/18 LI/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In some embodiments, the combination therapy as described herein are administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer). CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the combination therapy as described herein are administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Boorganic & Medicinal Chemistry Letters (2007) 17:617-620).

Exemplary RNAi and antisense RNA agents for treating cancer include, but not limited to, CALAA-01, siG12D LODER (Local Drug Elute®), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®)), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®), VIADUR™), megestrol (megestrol acetate, MEGACE®), nilutamide (ANANDRON®, NILANDRON®), octreotide (octreotide acetate. SANDOSTATIN®, SANDOSTATIN LAR®), raloxifene (EVISTA®), romiplostim (NPLATE®)), tamoxifen (NOVALDEX®), and toremifene (FARESTON®))), phospholipase A2 inhibitors (e.g., anagrelide (AGRYLIN®), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the combination therapy as described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®)), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010). MGCD-265, motesanib diphosphate (AMG-706), MP-470, OSI-930, Pazopanib Hydrochloride, PD173074, Sorafenib Tosylate (Bay 43-9006), SU 5402, TSU-68(SU6668), vatalanib, XL880 (GSK1363089. EXEL-2880). Selected tyrosine kinase inhibitors are chosen from sunitinib, erlotinib, gefitinib, or sorafenib. In some embodiments, the tyrosine kinase inhibitor is sunitinib.

In some embodiments, the combination therapy as described herein are administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab): VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the combination therapy as described herein. The methods can be used in a therapeutic protocol in vivo.

In some embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-91718) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In some embodiments, the inhibitor of PD-1 is an antibody molecule having a sequence substantially identical or similar thereto, e.g., a sequence at least 85%, 90° %, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In some embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.570, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In some embodiments, the PD-L1 inhibitor is an anti-PD-L1 antibody, e.g., YW243.55.S70. The YW243.55.S70 antibody is described, e.g., in WO 2010/077634. In some embodiments, the PD-L1 inhibitor is MDX-1105 (also called BMS-91719), which is described, e.g., in WO2007/005874. In some embodiments, the PD-LI inhibitor is MDPL3280A (Genentech/Roche), which is a human Fc-optimized IgGI monoclonal antibody against PD-L1. See, e.g., U.S. Pat. No. 7,943,743 and U.S Publication No.: 20120039906. In some embodiments, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In some embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In some embodiments, the immune checkpoint inhibitor is a LAG-3 inhibitor, e.g., an anti LAG-3 antibody molecule. In some embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### Method of Expanding Cells

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell, a CD1b restricted T cell, a CD1c restricted T cell, a GEM T cell, and a LDN5-like T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, the cell is CD4+/CD8−, CD4−/CD8+, CD4−/CD8−, or CD69+.

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In one embodiment, the methods as described herein can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). In some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, a TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a TEMRA cell or a TIL population) can be expanded by contacting the immune cell population with the multifunctional polypeptide molecule as described herein.

In some embodiments, the expansion occurs in vivo, e.g., in a subject. In some embodiments, a subject is administered the multifunctional polypeptide molecule as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multifunctional polypeptide molecule as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 100, 200, 300, 400, 500, 600, 700, 800, 900, 1000, 1500, 2000, 2500, 3000, 3500, 400, 4500, 5000, 5500, 6000, 6500, 7000, 7500, 8000, 8500, 9000, 9500, or 10000 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 1,6 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multifunctional polypeptide molecule as described herein can expand, e.g., selectively or preferentially expand, T cells expressing a T cell receptor (TCR) comprising a TCR alpha and/or TCR beta molecule, e.g., TCR alpha-beta T cells (αβ T cells). In some embodiments, the multifunctional polypeptide molecule as described herein does not expand, or induce proliferation of T cells expressing a TCR comprising a TCR gamma and/or TCR delta molecule, e.g., TCR gamma-delta T cells (γδ T cells). In some embodiments, the multifunctional polypeptide molecule as described herein selectively or preferentially expands αβ T cells over γδ T cells.

Without wishing to be bound by theory, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of ap T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

In some embodiments, the multifunctional polypeptide molecule as described herein results in expansion of an immune cell, e.g., a T cell, a tumor infiltrating lymphocyte (TIL), an NK cell, or other immune cells (e.g., as described herein).

In some embodiments, binding of the multifunctional polypeptide molecule as described herein to a TCRβ V region results in one, two, three or all of: (i) reduced T cell proliferation kinetics; (ii) cell killing, e.g., target cell killing, e.g. cancer cell killing, e.g., as measured by an assay of Example 4: (iii) increased Natural Killer (NK) cell proliferation, e.g., expansion; or (iv) expansion. e.g., at least about 1.1-10 expansion (e.g., at least about 1.1, 1.2, 1.3, 1.4, 1.5, 2, 3, 4, 5, 6, 7, 8, 9, or 10 fold expansion), of a population of T cells having a memory-like phenotype, e.g., as described herein, e.g., wherein (i)-(iv) are relative to the non-TCRβV-binding T cell engager.

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, expansion of the population of immune cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TCRβV antibody molecule or the multispecific or multifunctional molecules as described herein.

In some embodiments, expansion of the population of memory effector T cells, e.g., TEM cells, e.g., TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (ap T cells).

In some embodiments, the method results in expansion of αβT cells over expansion of T cells expressing a TCR comprising a TCR gamma and/or TCR delta molecule, e.g., TCR gamma-delta T cells (75 T cells). In some embodiments, expansion of αβT cells over γδ T cells results in reduced production of cytokines associated with CRS. In some embodiments, expansion of αβT cells over 76 T cells results in immune cells that have reduced capacity to, e.g., are less prone to, induce CRS upon administration into a subject.

In some embodiments, an immune cell population (e.g., T cells (e.g., TEMRA cells or TILs) or NK cells) cultured in the presence of, e.g., expanded with, the multifunctional polypeptide molecule as described herein does not induce CRS and/or NT when administered into a subject, e.g., a subject having a disease or condition as described herein.

In some embodiments, provided herein is a multifunctional polypeptide molecule as described herein comprising a non-murine, e.g., a human-like antibody molecule (e.g., a human or humanized antibody molecule), which binds, e.g., specifically binds, to a T cell receptor beta variable (TCRβV) region. In some embodiments, binding of the multifunctional polypeptide molecule as described herein results in expansion, e.g., at least about 1.1-50 fold expansion (e.g., at least about 1.5-40 fold, 2-35 fold, 3-30 fold. 5-25 fold, 8-20 fold, or 10-15 fold expansion), of a population of T cells, e.g., a population of T cells having a memory-like phenotype, e.g., CD45RA+ CCR7− T cells. In some embodiments, the population of T cells having a memory-like phenotype comprises CD4+ and/or CD8+ T cells. In some embodiments, the population of T cells having a memory-like phenotype comprises a population of memory T cells, e.g., T effector memory (TEM) cells, e.g., TEM cells expressing CD45RA (TEMRA) cells, e.g., CD4+ or CD8+ TEMRA cells. In some embodiments, the population of T cells having a memory-like phenotype does not express a senescent marker, e.g., CD57. In some embodiments, the population of T cells having a memory-like phenotype does not express an inhibitory receptor, e.g., OX40, 4-1BB, and/or ICOS.

In some embodiments, the population of T cells having a memory-like phenotype is a population of T cells with CD45RA+ CCR7− CD57−. In some embodiments, the population of T cells having a memory-like phenotype does not express an inhibitory receptor, e.g., OX40, 4-1BB, and/or ICOS.

In some embodiments, the population of T cells having a memory-like phenotype, e.g., as described herein, has increased proliferative capacity, e.g., as compared to a reference cell population, e.g., an otherwise similar population of cells that has not been contacted with an anti-TCRβV antibody or the multispecific or multifunctional molecules as described herein.

In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion).

In some embodiments, expansion of the population of T cells having a memory-like phenotype, e.g., memory effector T cells, e.g., TEM cells, e.g., TEMRA cells, e.g., CD4+ or CD8+ TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the population of expanded T cells having a memory-like phenotype, e.g., T effector memory cells, comprises cells T cells, e.g., CD3+, CD8+ or CD4+ T cells. In some embodiments, the population of expanded T cells having a memory-like phenotype. T effector memory cells, comprises CD3+ and CD8+ T cells. In some embodiments, the population of expanded T cells having a memory-like phenotype, e.g., T effector memory cells comprises CD3+ and CD4+ T cells.

In some embodiments, the population of expanded T cells having a memory-like phenotype, T effector memory (TEM) cells, comprises cells T cells, e.g., CD3+, CD8+ or CD4+T cells, which express or re-express, CD45RA, e.g., CD45RA+. In some embodiments, the population comprises TEM cells expressing CD45RA, e.g., TEMRA cells. In some embodiments, expression of CD45RA on TEMRA cells, e.g., CD4+ or CD8+ TEMRA cells, can be detected by a method as described herein, e.g., flow cytometry.

In some embodiments, the population of T cells having a memory-like phenotype, e.g., TEMRA cells have low or no expression of CCR7, e.g., CCR7− or CCR7 low. In some embodiments, expression of CCR7 on TEMRA cells cannot be detected by a method as described herein, e.g., flow cytometry.

In some embodiments, the population of T cells having a memory-like phenotype, e.g., TEMRA cells express CD95, e.g., CD95+. In some embodiments, expression of CD95 on TEMRA cells can be detected by a method as described herein, e.g., flow cytometry.

In some embodiments, the population of T cells having a memory-like phenotype. e.g., TEMRA cells express CD45RA, e.g., CD45RA+, have low or no expression of CCR7, e.g., CCR7− or CCR7 low, and express CD95, e.g., CD95+. In some embodiments, the population of T cells having a memory-like phenotype, e.g., TEMRA cells can be identified as CD45RA+, CCR7− and CD95+ cells. In some embodiments, the population of T cells having a memory-like phenotype, e.g., TEMRA cells comprise CD3+, CD4+ or CD8+ T cells (e.g., CD3+ T cells, CD3+ CD8+ T cells, or CD3+ CD4+ T cells).

In some embodiments, the population of T cells having a memory-like phenotype does not express a senescent marker, e.g., CD57.

In some embodiments, the population of T cells having a memory-like phenotype does not express an inhibitory receptor, e.g., OX40, 4-1BB, and/or ICOS.

In some embodiments, binding of the multifunctional polypeptide molecule as described herein results in expansion, e.g., at least about 1.1-50 fold expansion (e.g., at least about 1.5-40 fold. 2-35 fold, 3-30 fold, 5-25 fold, 8-20 fold, or 10-15 fold expansion), of a subpopulation of T cells. In some embodiments, the multifunctional polypeptide molecule as described herein-activated (e.g., expanded) subpopulation of T cells resemble TEMRA cells in high expression of CD45RA and/or low expression of CCR7. In some embodiments, the multifunctional polypeptide molecule as described herein-activated (e.g., expanded) subpopulation of T cells do not display upregulation of the senescence markers CD57 and/or KLRG1. In some embodiments, the multifunctional polypeptide molecule as described herein-activated (e.g., expanded) subpopulation of T cells do not display upregulation of co-stimulatory molecules CD27 and/or CD28. In some embodiments, the multifunctional polypeptide molecule as described herein-activated (e.g., expanded) subpopulation of T cells are highly proliferative. In some embodiments, the multifunctional polypeptide molecule as described herein-activated (e.g., expanded) subpopulation of T cells secrete IL-2. In some embodiments, expression of surface markers on T cells can be detected by a method as described herein, e.g., flow cytometry. In some embodiments, the proliferative capability of T cells can be detected by a method as described herein, e.g., a method described in Example 4. In some embodiments, cytokine expression of T cells can be detected by a method as described herein. In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion). In some embodiments, the expansion is compared to expansion of a similar population of cells with an antibody that binds to a CD3 molecule, e.g., CD3 epsilon (CD3e) molecule; or a TCR alpha (TCRα) molecule.

In some embodiments, binding of the multifunctional polypeptide molecule as described herein results in proliferation, e.g., expansion, e.g., at least about 1.1-50 fold expansion (e.g., at least about 1.540 fold, 2-35 fold, 3-30 fold, 5-25 fold, 8-20 fold, or 10-15 fold expansion), of a population of Natural Killer (NK) cells. In some embodiments, the expansion of NK cells is at least about 1.1-30 fold expansion (e.g., at least about 1.1, 1.2, 1.3, 1.4, 1.5, 2, 3, 4, 5, 6, 7, 8, 9, 10, 15, 20, 25, or at least about 1.1-5, 5-10, 10-15, 15-20, 20-25, or 25-30 fold expansion). In some embodiments, the expansion of NK cells is measure by an assay of Example 4. In some embodiments, the expansion of NK cells by, e.g., binding of, the multifunctional polypeptide molecule as described herein is compared to expansion of an otherwise similar population not contacted with the multifunctional polypeptide molecule as described herein.

In some embodiments, binding of the multifunctional polypeptide molecule as described herein results in cell killing, e.g., target cell killing, e.g. cancer cell killing. In some embodiments, the cancer cell is a hematological cancer cell or a solid tumor cell. In some embodiments, the cancer cell is a multiple myeloma cell. In some embodiments, binding of the multifunctional polypeptide molecule as described herein results in cell killing in vitro or in vivo. In some embodiments, cell killing is measured by an assay of Example 4.

In some embodiments, binding of the multifunctional polypeptide molecule as described herein to a TCRβV region results in an increase or decrease of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) of any of the activities described herein compared the activity of 16G8 or TM23 murine antibody, or a humanized version thereof as described in U.S. Pat. No. 5,861,155.

In some embodiments, the method comprises expanding, e.g., increasing the number of, an immune cell population in the subject. In some embodiments, provided herein is a method of expanding, e.g., increasing the number of, an immune cell population comprising, contacting the immune cell population with an effective amount of the multifunctional polypeptide molecule as described herein. In some embodiments, the expansion occurs in vivo or ex vivo (e.g., in vitro).

In some embodiments, provided herein is a method of expanding, e.g., increasing the number of, an immune cell population comprising, contacting the immune cell population with a multifunctional polypeptide molecule as described herein comprising an antibody molecule, e.g., humanized antibody molecule, which binds, e.g., specifically binds, to a T cell receptor beta variable chain (TCRβV) region (e.g., anti-TCRβV antibody molecule) or specifically binds, to a T cell receptor beta variable chain (TCRαV) region (e.g., anti-TCRαV antibody molecule), thereby expanding the immune cell population. In some embodiments, the expansion occurs in vivo or ex vivo (e.g., in vitro).

In some embodiments, provided herein is a method of expanding a population of immune effector cells from a subject having a cancer, the method comprising: (i) isolating a biological sample comprising a population of immune effector cells from the subject; e.g., a peripheral blood sample, biopsy sample, or bone marrow sample; (ii) acquiring a value of the status of one or more TCRαV molecules or TCRαV molecules for the subject, e.g., in the biological sample from the subject, wherein said value comprises a measure of the presence of, e.g., level or activity of, a TCRβV molecule or a TCRαV molecule in a sample from the subject compared to a reference value, e.g., a sample from a health subject, wherein a value that is higher, e.g., increased, in the subject relative to the reference, e.g., healthy subject, is indicative of the presence of cancer in the subject, and (iii) contacting the biological sample comprising a population of immune effector cells with the multifunctional polypeptide molecule as described herein.

In some embodiments, the method further comprises administering the population of immune effector cells contacted with the multifunctional polypeptide molecule as described herein to the subject.

In some embodiments, a higher, e.g., increased, level or activity of one or more TCRαV molecules or TCRαV molecules in a subject, e.g., in a sample from a subject, is indicative of a bias, e.g., a preferential expansion, e.g., clonal expansion, of T cells expressing said one or more TCRβV molecules or TCRαV molecules in the subject.

Accordingly, provided herein are, inter alia, multispecific or multifunctional molecules comprising TCRαV-binding or TCRαV-binding moieties as described herein (e.g., multispecific or multifunctional antibody molecules) that comprise anti-TCRαV antibody molecules or anti-TCRαV antibody molecules, nucleic acids encoding the same, methods of producing the aforesaid molecules, pharmaceutical compositions comprising aforesaid molecules, and methods of treating a disease or disorder, e.g., cancer, using the aforesaid molecules. The antibody molecules and pharmaceutical compositions as described herein can be used (alone or in combination with other agents or therapeutic modalities) to treat, prevent and/or diagnose disorders and conditions, e.g., cancer, e.g., as described herein.

TABLE 21
Amino acid and nucleotide sequences for murine, chimeric and humanized antibody
molecules which bind to TCRVB 6, e.g., TCRVB 6-5, TCRVB6-2. The antibody molecules
include murine mAb Antibody A, and humanized mAb Antibody A-H Clones A-H.1 to A-H.85.
The amino acid the heavy and light chain CDRs, and the amino acid and nucleotide sequences of
the heavy and light chain variable regions, and the heavy and light chains are shown.
Antibody A (murine), also referred to as H131, TCRVB 6-2 binder

| | | |
|---|---|---|
| SEQ ID NO: | HC CDR1 | GYSFTTYYIH |
| 1003 | (Combined) | |
| SEQ ID NO: | HC CDR2 | WFFPGSGNIKYNEKFKG |
| 1004 | (Combined) | |
| SEQ ID NO: | HC CDR3 | SYYSYDVLDY |
| 1005 | (Combined) | |
| SEQ ID NO: | HC CDR1 (Kabat) | TYYIH |
| 1045 | | |
| SEQ ID NO: | HC CDR2 (Kabat) | WFFPGSGNIKYNEKFKG |
| 1004 | | |
| SEQ ID NO: | HC CDR3 (Kabat) | SYYSYDVLDY |
| 1005 | | |
| SEQ ID NO: | HC CDR1 (Chothia) | GYSFTTY |
| 1015 | | |
| SEQ ID NO: | HC CDR2 (Chothia) | FPGSGN |
| 1016 | | |
| SEQ ID NO: | HC CDR3 (Chothia) | SYYSYDVLDY |
| 1005 | | |
| SEQ ID NO: | VH | QVQLQQSGPELVKPGTSVKISCKASGYSFTTYYIHWVK |
| 1001 | | QRPGQGLEWIGWFFPGSGNIKYNEKFKGKATLTADTSS |
| | | STAYMQLSSLTSEESAVYFCAGSYYSYDVLDYWGHGT |
| | | TLTVSS |
| SEQ ID NO: | LC CDR1 | KASQNVGINVV |
| 1006 | (Combined) | |
| SEQ ID NO: | LC CDR2 | SSSHRYS |
| 1007 | (Combined) | |
| SEQ ID NO: | LC CDR3 | QQFKSYPLT |
| 1008 | (Combined) | |
| SEQ ID NO: | LC CDR1 (Kabat) | KASQNVGINVV |
| 1006 | | |
| SEQ ID NO: | LC CDR2 (Kabat) | SSSHRYS |
| 1007 | | |
| SEQ ID NO: | LC CDR3 (Kabat) | QQFKSYPLT |
| 1008 | | |
| SEQ ID NO: | LC CDR1 (Chothia) | KASQNVGINVV |
| 1006 | | |
| SEQ ID NO: | LC CDR2 (Chothia) | SSSHRYS |
| 1007 | | |
| SEQ ID NO: | LC CDR3 (Chothia) | QQFKSYPLT |
| 1008 | | |
| SEQ ID NO: | VL | DILMTQSQKFMSTSLGDRVSVSCKASQNVGINVVWHQ |
| 1002 | | QKPGQSPKALIYSSSHRYSGVPDRFTGSGSGTDFTLTIN |
| | | NVQSEDLAEYFCQQFKSYPLTFGAGTKLELK |

Antibody A humanized (A-H antibody), TCRVB 6-2 binder
A-H.1 antibody (also referred to as BHM1709)

| | | |
|---|---|---|
| SEQ ID NO: | HC CDR1 | GYSFTTYYIH |
| 1003 | (Combined) | |
| SEQ ID NO: | HC CDR2 | WFFPGSGNIKYNEKFKG |
| 1004 | (Combined) | |
| SEQ ID NO: | HC CDR3 | SYYSYDVLDY |
| 1005 | (Combined) | |
| SEQ ID NO: | VH | QVQLVQSGAEVKKPGSSVKVSCKASGYSFTTYYIHWV |
| 1009 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |
| SEQ ID NO: | DNA VH | CAGGTGCAGCTGGTTCAGTCTGGCGCCGAAGTGAAG |
| 1012 | | AAACCTGGCTCCTCCGTGAAGGTGTCCTGCAAGGCTT |
| | | CCGGCTACTCCTTCACCACCTACTACATCCACTGGGT |
| | | CCGACAGGCCCCTGGACAAGGATTGGAATGGATGGG |
| | | CTGGTTCTTCCCCGGCTCCGGCAACATCAAGTACAAC |
| | | GAGAAGTTCAAGGGCCGCGTGACCATCACCGCCGAC |
| | | ACCTCTACCTCTACCGCCTACATGGAACTGTCCAGCC |
| | | TGAGATCTGAGGACACCGCCGTGTACTACTGCGCCG |
| | | GCTCCTACTACTCTTACGACGTGCTGGATTACTGGGG |
| | | CCAGGGCACCACAGTGACAGTGTCCTCT |
| SEQ ID NO: | VH-IgM constant | METDTLLLWVLLLWVPGSTGQVQLVQSGAEVKKPGSS |
| 1017 | delta CDC | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSGSASAPTLFPL |
| | | VSCENSPSDTSSVAVGCLAQDFLPDSITFSWKYKNNSDI |
| | | SSTRGFPSVLRGGKYAATSQVLLPSKDVMQGTDEHVV |
| | | CKVQHPNGNKEKNVPLPVIAELPPKVSVFVPPRDGFFG |
| | | NPRKSKLICQATGFSPRQIQVSWLREGKQVGSGVTTDQ |
| | | VQAEAKESGPTTYKVTSTLTIKESDWLGQSMFTCRVDH |
| | | RGLTFQQNASSMCVPDQDTAIRVFAIPPSFASIFLTKSTK |
| | | LTCLVTDLTTYDSVTISWTRQNGEAVKTHTNISESHPNA |
| | | TFSAVGEASICEDDWNSGERFTCTVTHTDLASSLKQTIS |
| | | RPKGVALHRPDVYLLPPAREQLNLRESATITCLVTGFSP |
| | | ADVFVQWMQRGQPLSPEKYVTSAPMPEPQAPGRYFAH |
| | | SILTVSEEEWNTGETYTCVVAHEALPNRVTERTVDKST |
| | | GKPTLYNVSLVMSDTAGTCY |
| SEQ ID NO: | VH-IgGA1 | METDTLLLWVLLLWVPGSTGQVQLVQSGAEVKKPGSS |
| 1018 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASPTSPKVFPLS |
| | | LCSTQPDGNVVIACLVQGFFPQEPLSVTWSESGQGVTA |
| | | RNFPPSQDASGDLYTTSSQLTLPATQCLAGKSVTCHVK |
| | | HYTNPSQDVTVPCPVPSTPPTPSPSTPPTPSPSCCHPRLSL |
| | | HRPALEDLLLGSEANLTCTLTGLRDASGVTFTWTPSSG |
| | | KSAVQGPPERDLCGCYSVSSVLPGCAEPWNHGKTFTCT |
| | | AAYPESKTPLTATLSKSGNTFRPEVHLLPPPSEELALNEL |
| | | VTLTCLARGFSPKDVLVRWLQGSQELPREKYLTWASR |
| | | QEPSQGTTTFAVTSILRVAAEDWKKGDTFSCMVGHEAL |
| | | PLAFTQKTIDRLAGKPTHVNVSVVMAEVDGTCY |
| SEQ ID NO: | VH-IgGA2 | METDTLLLWVLLLWVPGSTGQVQLVQSGAEVKKPGSS |
| 1019 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASPTSPKVFPLS |
| | | LDSTPQDGNVVVACLVQGFFPQEPLSVTWSESGQNVTA |
| | | RNFPPSQDASGDLYTTSSQLTLPATQCPDGKSVTCHVK |
| | | HYTNSSQDVTVPCRVPPPPPCCHPRLSLHRPALEDLLLG |
| | | SEANLTCTLTGLRDASGATFTWTPSSGKSAVQGPPERD |
| | | LCGCYSVSSVLPGCAQPWNHGETFTCTAAHPELKTPLT |
| | | ANITKSGNTFRPEVHLLPPPSEELALNELVTLTCLARGFS |
| | | PKDVLVRWLQGSQELPREKYLTWASRQEPSQGTTTYA |
| | | VTSILRVAAEDWKKGETFSCMVGHEALPLAFTQKTIDR |
| | | MAGKPTHINVSVVMAEADGTCY |
| SEQ ID NO: | Heavy chain | METDTLLLWVLLLWVPGSTGQVQLVQSGAEVKKPGSS |
| 1020 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASTKGPSVFPL |
| | | APSSKSTSGGTAALGCLVKDYFPEPVTVSWNSGALTSG |
| | | VHTFPAVLQSSGLYSLSSVVTVPSSSLGTQTYICNVNHK |
| | | PSNTKVDKRVEPKSCDKTHTCPPCPAPELLGGPSVFLFP |
| | | PKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVYTLPPSREE |
| | | MTKNQVSLTCLVKGFYPSDIAVEWESNGQPENNYKTTP |
| | | PVLDSDGSFFLYSKLTVDKSRWQQGNVFSCSVMHEAL |
| | | HNHYTQKSLSLSPGK |
| SEQ ID NO: | LC CDR1 | KASQNVGINVV |
| 1006 | (Combined) | |
| SEQ ID NO: | LC CDR2 | SSSHRYS |
| 1007 | (Combined) | |
| SEQ ID NO: | LC CDR3 | QQFKSYPLT |
| 1008 | (Combined) | |
| SEQ ID NO: | VL | DIQMTQSPSFLSASVGDRVTITCKASQNVGINVVWHQQ |
| 1010 | | KPGKAPKALIYSSSHRYSGVPSRFSGSGSGTEFTLTISSL |
| | | QPEDFATYFCQQFKSYPLTFGQGTKLEIK |
| SEQ ID NO: | DNA VL | GACATCCAGATGACCCAGTCTCCATCCTTCCTGTCCG |
| 1013 | | CCTCTGTGGGCGACAGAGTGACCATCACATGCAAGG |
| | | CCTCTCAGAACGTGGGCATCAACGTCGTGTGGCACCA |
| | | GCAGAAGCCTGGCAAGGCTCCTAAGGCTCTGATCTA |
| | | CTCCTCCAGCCACCGGTACTCTGGCGTGCCCTCTAGA |
| | | TTTTCCGGCTCTGGCTCTGGCACCGAGTTTACCCTGA |
| | | CAATCTCCAGCCTGCAGCCTGAGGACTTCGCCACCTA |
| | | CTTTTGCCAGCAGTTCAAGAGCTACCCTCTGACCTTT |
| | | GGCCAGGGCACCAAGCTGGAAATCAAG |
| SEQ ID NO: | VL and kappa | METDTLLLWVLLLWVPGSTGDIQMTQSPSFLSASVGDR |
| 1023 | constant region/light | VTITCKASQNVGINVVWHQQKPGKAPKALIYSSSHRYS |
| | chain | GVPSRFSGSGSGTEFTLTISSLQPEDFATYFCQQFKSYPL |
| | | TFGQGTKLEIKRTVAAPSVFIFPPSDEQLKSGTASVVCLL |
| | | NNFYPREAKVQWKVDNALQSGNSQESVTEQDSKDSTY |
| | | SLSSTLTLSKADYEKHKVYACEVTHQGLSSPVTKSFNR |
| | | GEC |

A-H.2 antibody (also referred to as BHM1710)

| | | |
|---|---|---|
| SEQ ID NO: | HC CDR1 | GYSFTTYYIH |
| 1003 | (Combined) | |
| SEQ ID NO: | HC CDR2 | WFFPGSGNIKYNEKFKG |
| 1004 | (Combined) | |
| SEQ ID NO: | HC CDR3 | SYYSYDVLDY |
| 1005 | (Combined) | |
| SEQ ID NO: | VH | QVQLVQSGAEVKKPGSSVKVSCKASGYSFTTYYIHWV |
| 1009 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |
| SEQ ID NO: | DNA VH | CAGGTGCAGCTGGTTCAGTCTGGCGCCGAAGTGAAG |
| 1012 | | AAACCTGGCTCCTCCGTGAAGGTGTCCTGCAAGGCTT |
| | | CCGGCTACTCCTTCACCACCTACTACATCCACTGGGT |
| | | CCGACAGGCCCCTGGACAAGGATTGGAATGGATGGG |
| | | CTGGTTCTTCCCCGGCTCCGGCAACATCAAGTACAAC |
| | | GAGAAGTTCAAGGGCCGCGTGACCATCACCGCCGAC |
| | | ACCTCTACCTCTACCGCCTACATGGAACTGTCCAGCC |
| | | TGAGATCTGAGGACACCGCCGTGTACTACTGCGCCG |
| | | GCTCCTACTACTCTTACGACGTGCTGGATTACTGGGG |
| | | CCAGGGCACCACAGTGACAGTGTCCTCT |
| SEQ ID NO: | Heavy chain | METDTLLLWVLLLWVPGSTGQVQLVQSGAEVKKPGSS |
| 1020 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASTKGPSVFPL |
| | | APSSKSTSGGTAALGCLVKDYFPEPVTVSWNSGALTSG |
| | | VHTFPAVLQSSGLYSLSSVVTVPSSSLGTQTYICNVNHK |
| | | PSNTKVDKRVEPKSCDKTHTCPPCPAPELLGGPSVFLFP |
| | | PKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVYTLPPSREE |
| | | MTKNQVSLTCLVKGFYPSDIAVEWESNGQPENNYKTTP |
| | | PVLDSDGSFFLYSKLTVDKSRWQQGNVFSCSVMHEAL |
| | | HNHYTQKSLSLSPGK |
| SEQ ID NO: | LC CDR1 | KASQNVGINVV |
| 1006 | (Combined) | |
| SEQ ID NO: | LC CDR2 | SSSHRYS |
| 1007 | (Combined) | |
| SEQ ID NO: | LC CDR3 | QQFKSYPLT |
| 1008 | (Combined) | |
| SEQ ID NO: | VL | DIQMTQSPSSLSASVGDRVTITCKASQNVGINVVWHQQ |
| 1011 | | KPGKVPKALIYSSSHRYSGVPSRFSGSGSGTDFTLTISSL |
| | | QPEDVATYFCQQFKSYPLTFGQGTKLEIK |
| SEQ ID NO: | DNA VL | GACATCCAGATGACCCAGTCTCCATCCTCTCTGTCCG |
| 1014 | | CCTCTGTGGGCGACAGAGTGACCATCACATGCAAGG |
| | | CCTCTCAGAACGTGGGCATCAACGTCGTGTGGCACCA |
| | | GCAGAAACCTGGCAAGGTGCCCAAGGCTCTGATCTA |
| | | CTCCTCCAGCCACAGATACTCCGGCGTGCCCTCTAGA |
| | | TTCTCCGGCTCTGGCTCTGGCACCGACTTTACCCTGA |
| | | CAATCTCCAGCCTGCAGCCTGAGGACGTGGCCACCTA |
| | | CTTTTGCCAGCAGTTCAAGAGCTACCCTCTGACCTTT |
| | | GGCCAGGGCACCAAGCTGGAAATCAAG |
| SEQ ID NO: | Light chain | METDTLLLWVLLLWVPGSTGDIQMTQSPSSLSASVGDR |
| 1021 | | VTITCKASQNVGINVVWHQQKPGKVPKALIYSSSHRYS |
| | | GVPSRFSGSGSGTDFTLTISSLQPEDVATYFCQQFKSYPL |
| | | TFGQGTKLEIKRTVAAPSVFIFPPSDEQLKSGTASVVCLL |
| | | NNFYPREAKVQWKVDNALQSGNSQESVTEQDSKDSTY |
| | | SLSSTLTLSKADYEKHKVYACEVTHQGLSSPVTKSFNR |
| | | GEC |

A-H.3 antibody

| | | |
|---|---|---|
| SEQ ID NO: | VH + VL | QVQLVQSGAEVKKPGSSVKVSCKASGTDFKLTYIHWV |
| 1022 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 1024 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1025 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSS |

A-H.4

| | | |
|---|---|---|
| SEQ ID NO: | VH + VL | QVQLVQSGAEVKKPGSSVKVSCKASGTDFDKIYIHWVR |
| 1026 | | QAPGQGLEWMGRISAGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVEDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 1024 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 1027 | | QAPGQGLEWMGRISAGSGNVKYNEKFKGRVTITADTS |
| | | TSTAYMELSSLRSEDTAVYYCAGSYYSYDVLDYWGQG |
| | | TTVTVSS |
| A-H.5 | | |
| SEQ ID NO: | VH + VL | QVQLVQSGAEVKKPGSSVKVSCKASGHDFRDFYIHWV |
| 1028 | | RQAPGQGLEWMGRVYPGSGSYRYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFRDFYIHWV |
| 1030 | | RQAPGQGLEWMGRVYPGSGSYRYNEKFKGRVTITADT |
| | | STSTAYMELSSLRSEDTAVYYCAGSYYSYDVLDYWGQ |
| | | GTTVTVSS |

A-H.6

| | | |
|---|---|---|
| SEQ ID NO: | VH + VL | QVQLVQSGAEVKKPGSSVKVSCKASGHDFKLTYIHWV |
| 1031 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWYQQ |
| 1032 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFKLTYIHWV |
| 1033 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.7

| | | |
|---|---|---|
| SEQ ID NO: | VH + VL | QVQLVQSGAEVKKPGSSVKVSCKASGTDFKLTYIHWV |
| 1034 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVENKVAWHQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVENKVAWHQQ |
| 1035 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1036 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |

A-H.8

| | | |
|---|---|---|
| SEQ ID NO: | VH + VL | QVQLVQSGAEVKKPGSSVKVSCKASGTDFDKIYIHWVR |
| 1046 | | QAPGQGLEWMGRIFAGSGNTKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVDDRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 1047 | | QAPGQGLEWMGRIFAGSGNTKYNEKFKGRVTITADTST |
| | | STAYMELSSLRSEDTAVYYCAGSYYSYDVLDYWGQGT |
| | | TVTVSS |

A-H.9

| | | |
|---|---|---|
| SEQ ID NO: | VH + VL | QVQLVQSGAEVKKPGSSVKVSCKASGHDFDKFYIHWV |
| 1048 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGNRVAWYQQKPGKAPKAL |
| | | IYSSSHRYSGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 1049 | | KPGKAPKALIYSSSHRYSGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 1050 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.10

| | | |
|---|---|---|
| SEQ ID NO: | VH + VL | QVQLVQSGAEVKKPGSSVKVSCKASGHDFDKFYIHWV |
| 1051 | | RQAPGQGLEWMGRIFAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGDRVAWYQQKPGKAPKAL |
| | | IYSSSHRYKGVPSRFSGSGSGTEFTLTISSLQPEDFATYFC |
| | | QQFKSYPLTFGQGTKLEIKS |
| SEQ ID NO: | VL | DIQMTQSPSFLSASVGDRVTITCKASQNVGDRVAWYQQ |
| 1052 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 1053 | | RQAPGQGLEWMGRIFAGSGNVKYNEKFKGRVTITADT |
| | | STSTAYMELSSLRSEDTAVYYCAGSYYSYDVLDYWGQ |
| | | GTTVTVSS |

A-H.11

| | | |
|---|---|---|
| SEQ ID NO: | VH + VL | QVQLVQSGAEVKKPGSSVKVSCKASGTDFKLTYIHWV |
| 1054 | | RQAPGQGLEWMGRVSPGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 1055 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1056 | | RQAPGQGLEWMGRVSPGSGNVKYNEKFKGRVTITADT |
| | | STSTAYMELSSLRSEDTAVYYCAGSYYSYDVLDYWGQ |
| | | GTTVTVSS |

A-H.12

| | | |
|---|---|---|
| SEQ ID NO: | VH + VL | QVQLVQSGAEVKKPGSSVKVSCKASGTDFDKIYIHWVR |
| 1057 | | QAPGQGLEWMGRVSAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGNRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 1058 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 1059 | | QAPGQGLEWMGRVSAGSGNTKYNEKFKGRVTITADTS |
| | | TSTAYMELSSLRSEDTAVYYCAGSYYSYDVLDYWGQG |
| | | TTVTVSS |

A-H.13, also referred to as A-H.69

| | | |
|---|---|---|
| SEQ ID NO: | VH + VL | QVQLVQSGAEVKKPGSSVKVSCKASGTDFKLTYIHWV |
| 1060 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDNRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWYQQ |
| 1032 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1036 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |

A-H.14

| | | |
|---|---|---|
| SEQ ID NO: | VH + VL | QVQLVQSGAEVKKPGSSVKVSCKASGTDFDKIYIHWVR |
| 1037 | | QAPGQGLEWMGRISAGSGNTKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVDDRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 1038 | | QAPGQGLEWMGRISAGSGNTKYNEKFKGRVTITADTST |
| | | STAYMELSSLRSEDTAVYYCAGSYYSYDVLDYWGQGT |
| | | TVTVSS |

A-H.15

| | | |
|---|---|---|
| SEQ ID NO: | VH + VL | QVQLVQSGAEVKKPGSSVKVSCKASGTDFRLTYIHWV |
| 1061 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNKVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNKVAWHQ |
| 1062 | | QKPGKAPKALIYSSSHRYKGVPSRFSGSGSGTEFTLTISS |
| | | LQPEDFATYFCQQFKSYPLTFGQGTKLEIK |
| SEQ ID NO: | VH | QVQLVQSGAEVKKPGSSVKVSCKASGTDFRLTYIHWV |
| 1063 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSS |

A-H.16

| | | |
|---|---|---|
| SEQ ID NO: | VH + VL | QVQLVQSGAEVKKPGSSVKVSCKASGGTFRLTYIHWV |
| 1064 | | RQAPGQGLEWMGRVYPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFRLTYIHWV |
| 1065 | | RQAPGQGLEWMGRVYPGSGNTKYNEKFKGRVTITADT |
| | | STSTAYMELSSLRSEDTAVYYCAGSYYSYDVLDYWGQ |
| | | GTTVTVSS |

A-H.17

| | | |
|---|---|---|
| SEQ ID NO: | VH + VL | QVQLVQSGAEVKKPGSSVKVSCKASGTDFRLTYIHWV |
| 1066 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFRLTYIHWV |
| 1067 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSS |

A-H.18

| | | |
|---|---|---|
| SEQ ID NO: | VH + VL | QVQLVQSGAEVKKPGSSVKVSCKASGTDFKLTYIHWV |
| 1068 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVEDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 1024 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1036 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |

A-H.19

| | | |
|---|---|---|
| SEQ ID NO: | VH + VL | QVQLVQSGAEVKKPGSSVKVSCKASGGTFRLTYIHWV |
| 1069 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 1055 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFRLTYIHWV |
| 1070 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.20

| | | |
|---|---|---|
| SEQ ID NO: | VH + VL | QVQLVQSGAEVKKPGSSVKVSCKASGGTFDKTYIHWV |
| 1071 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFDKTYIHWV |
| 1072 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSS |

A-H.21

| | | |
|---|---|---|
| SEQ ID NO: | VH + VL | QVQLVQSGAEVKKPGSSVKVSCKASGHDFDKFYIHWV |
| 1077 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 1078 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSS |

A-H.22

| | | |
|---|---|---|
| SEQ ID NO: | VH + VL | QVQLVQSGAEVKKPGSSVKVSCKASGTDFKLTYIHWV |
| 1079 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNKVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNKVAWHQ |
| 1062 | | QKPGKAPKALIYSSSHRYKGVPSRFSGSGSGTEFTLTISS |
| | | LQPEDFATYFCQQFKSYPLTFGQGTKLEIK |
| SEQ ID NO: | VH | QVQLVQSGAEVKKPGSSVKVSCKASGTDFKLTYIHWV |
| 1080 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.23

| | | |
|---|---|---|
| SEQ ID NO: | VH + VL | QVQLVQSGAEVKKPGSSVKVSCKASGHDFRLTYIHWV |
| 1081 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVADRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVADRVAWYQQ |
| 1082 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFRLTYIHWV |
| 1083 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.24

| | | |
|---|---|---|
| SEQ ID NO: | VH + VL | QVQLVQSGAEVKKPGSSVKVSCKASGHDFHLWYIHWV |
| 1084 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNKVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNKVAWHQ |
| 1062 | | QKPGKAPKALIYSSSHRYKGVPSRFSGSGSGTEFTLTISS |
| | | LQPEDFATYFCQQFKSYPLTFGQGTKLEIK |
| SEQ ID NO: | VH | QVQLVQSGAEVKKPGSSVKVSCKASGHDFHLWYIHWV |
| 1085 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.25

| | | |
|---|---|---|
| SEQ ID NO: | VH + VL | QVQLVQSGAEVKKPGSSVKVSCKASGHDFHLWYIHWV |
| 1086 | | RQAPGQGLEWMGRVFAGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDKVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDKVAWYQQ |
| 1087 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFHLWYIHWV |
| 1088 | | RQAPGQGLEWMGRVFAGSGNTKYNEKFKGRVTITADT |
| | | STSTAYMELSSLRSEDTAVYYCAGSYYSYDVLDYWGQ |
| | | GTTVTVSS |

A-H.26

| | | |
|---|---|---|
| SEQ ID NO: | VH + VL | QVQLVQSGAEVKKPGSSVKVSCKASGTDFKLTYIHWV |
| 1089 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1090 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSS |

A-H.27

| | | |
|---|---|---|
| SEQ ID NO: | VH + VL | QVQLVQSGAEVKKPGSSVKVSCKASGTDFKLTYIHWV |
| 1091 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGNRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 1058 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1092 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.28

| | | |
|---|---|---|
| SEQ ID NO: | VH + VL | QVQLVQSGAEVKKPGSSVKVSCKASGTDFKLTYIHWV |
| 1093 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 1055 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1094 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSS |

A-H.29

| | | |
|---|---|---|
| SEQ ID NO: | VH + VL | QVQLVQSGAEVKKPGSSVKVSCKASGHDFHLWYIHWV |
| 1095 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGDRVAWHQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWHQQ |
| 1096 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFHLWYIHWV |
| 1097 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSS |

A-H.31

| | | |
|---|---|---|
| SEQ ID NO: | VH + VL | QVQLVQSGAEVKKPGSSVKVSCKASGHDFKLTYIHWV |
| 1098 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFKLTYIHWV |
| 1033 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.31

| | | |
|---|---|---|
| SEQ ID NO: | VH + VL | QVQLVQSGAEVKKPGSSVKVSCKASGTDFHLWYIHWV |
| 1099 | | RQAPGQGLEWMGRVFAGSGSYRYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFHLWYIHWV |
| 1100 | | RQAPGQGLEWMGRVFAGSGSYRYNEKFKGRVTITADT |
| | | STSTAYMELSSLRSEDTAVYYCAGSYYSYDVLDYWGQ |
| | | GTTVTVSS |

A-H.32

| | | |
|---|---|---|
| SEQ ID NO: | VH + VL | QVQLVQSGAEVKKPGSSVKVSCKASGTDFDKIYIHWVR |
| 1101 | | QAPGQGLEWMGRISAGSGNTKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVADRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVADRVAWYQQ |
| 1102 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 1038 | | QAPGQGLEWMGRISAGSGNTKYNEKFKGRVTITADTST |
| | | STAYMELSSLRSEDTAVYYCAGSYYSYDVLDYWGQGT |
| | | TVTVSS |

A-H.33

| | | |
|---|---|---|
| SEQ ID NO: | VH + VL | QVQLVQSGAEVKKPGSSVKVSCKASGTDFKLTYIHWV |
| 1103 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVEDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 1024 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1104 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSS |

A-H.34

| | | |
|---|---|---|
| SEQ ID NO: | VH + VL | QVQLVQSGAEVKKPGSSVKVSCKASGTDFRLTYIHWV |
| 1105 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGNRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 1058 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFRLTYIHWV |
| 1106 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSS |

A-H.35

| | | |
|---|---|---|
| SEQ ID NO: | VH + VL | QVQLVQSGAEVKKPGSSVKVSCKASGHDFDKTYIHWV |
| 1107 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 1024 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKTYIHWV |
| 1108 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.36

| | | |
|---|---|---|
| SEQ ID NO: | VH + VL | QVQLVQSGAEVKKPGSSVKVSCKASGHDFKLTYIHWV |
| 1109 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDRVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWHQQ |
| 1110 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFKLTYIHWV |
| 1111 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSS |

A-H.37

| | | |
|---|---|---|
| SEQ ID NO: | VH + VL | QVQLVQSGAEVKKPGSSVKVSCKASGHDFDKTYIHWV |
| 1112 | | RQAPGQGLEWMGRIYPGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVADRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVADRVAWYQQ |
| 1102 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDEDKTYIHWV |
| 1113 | | RQAPGQGLEWMGRIYPGSGNVKYNEKFKGRVTITADT |
| | | STSTAYMELSSLRSEDTAVYYCAGSYYSYDVLDYWGQ |
| | | GTTVTVSS |

A-H.38

| | | |
|---|---|---|
| SEQ ID NO: | VH + VL | QVQLVQSGAEVKKPGSSVKVSCKASGTDFDKTYIHWV |
| 1114 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKTYIHWV |
| 1115 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.39

| | | |
|---|---|---|
| SEQ ID NO: | VH + VL | QVQLVQSGAEVKKPGSSVKVSCKASGTDFDKIYIHWVR |
| 1116 | | QAPGQGLEWMGRISAGSGNIKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVDDRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 1117 | | QAPGQGLEWMGRISAGSGNIKYNEKFKGRVTITADTST |
| | | STAYMELSSLRSEDTAVYYCAGSYYSYDVLDYWGQGT |
| | | TVTVSS |

A-H.40

| | | |
|---|---|---|
| SEQ ID NO: | VH + VL | QVQLVQSGAEVKKPGSSVKVSCKASGTDFDKIYIHWVR |
| 1118 | | QAPGQGLEWMGRISAGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 1055 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 1027 | | QAPGQGLEWMGRISAGSGNVKYNEKFKGRVTITADTS |
| | | TSTAYMELSSLRSEDTAVYYCAGSYYSYDVLDYWGQG |
| | | TTVTVSS |

A-H.41

| | | |
|---|---|---|
| SEQ ID NO: | VH + VL | QVQLVQSGAEVKKPGSSVKVSCKASGGTFKLTYIHWV |
| 1119 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 1029 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFKLTYIHWV |
| 1120 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.42

| | | |
|---|---|---|
| SEQ ID NO: | VH + VL | QVQLVQSGAEVKKPGSSVKVSCKASGTDFKLTYIHWV |
| 1121 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDNRVAWHQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWHQQ |
| 1122 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 1123 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSS |

A-H.43

| | | |
|---|---|---|
| SEQ ID NO: | VH + VL | QVQLVQSGAEVKKPGSSVKVSCKASGHDFDKFYIHWV |
| 1124 | | RQAPGQGLEWMGRVSAGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWYQQ |
| 1032 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 1125 | | RQAPGQGLEWMGRVSAGSGNTKYNEKFKGRVTITADT |
| | | STSTAYMELSSLRSEDTAVYYCAGSYYSYDVLDYWGQ |
| | | GTTVTVSS |

A-H.44

| | | |
|---|---|---|
| SEQ ID NO: | VH + VL | QVQLVQSGAEVKKPGSSVKVSCKASGTDFDKFYIHWV |
| 1126 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGDRVVWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGTDFDKFYIHWV |
| 1127 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.45

| | | |
|---|---|---|
| SEQ ID NO: | VH + VL | QVQLVQSGAEVKKPGSSVKVSCKASGYSFTTYYIHWV |
| 1128 | | RQAPGQGLEWMGWFSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 1129 | | RQAPGQGLEWMGWFSAGSGNTKYNEKFKGRVTITADT |
| | | STSTAYMELSSLRSEDTAVYYCAVSYYSYDVLDYWGQ |
| | | GTTVTVSS |

A-H.46

| | | |
|---|---|---|
| SEQ ID NO: | VH + VL | QVQLVQSGAEVKKPGSSVKVSCKASGYSFTTYYIHWV |
| 1130 | | RQAPGQGLEWMGWFSAGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 1131 | | RQAPGQGLEWMGWFSAGSGNTKYNEKFKGRVTITADT |
| | | STSTAYMELSSLRSEDTAVYYCAGSYYSYDVLDYWGQ |
| | | GTTVTVSS |

A-H.47

| | | |
|---|---|---|
| SEQ ID NO: | VH + VL | QVQLVQSGAEVKKPGSSVKVSCKASGYSFTTYYIHWV |
| 1132 | | RQAPGQGLEWMGWFFPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 1133 | | RQAPGQGLEWMGWFFPGSGNTKYNEKFKGRVTITADT |
| | | STSTAYMELSSLRSEDTAVYYCAGSYYSYDVLDYWGQ |
| | | GTTVTVSS |

A-H.48

| | | |
|---|---|---|
| SEQ ID NO: | VH + VL | QVQLVQSGAEVKKPGSSVKVSCKASGYSFTTYYIHWV |
| 1134 | | RQAPGQGLEWMGWFSPGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 1135 | | RQAPGQGLEWMGWFSPGSGNTKYNEKFKGRVTITADT |
| | | STSTAYMELSSLRSEDTAVYYCAVSYYSYDVLDYWGQ |
| | | GTTVTVSS |

A-H.49

| | | |
|---|---|---|
| SEQ ID NO: | VH + VL | QVQLVQSGAEVKKPGSSVKVSCKASGYSFTTYYIHWV |
| 1136 | | RQAPGQGLEWMGWFSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 1137 | | RQAPGQGLEWMGWFSPGSGNTKYNEKFKGRVTITADT |
| | | STSTAYMELSSLRSEDTAVYYCAGSYYSYDVLDYWGQ |
| | | GTTVTVSS |

A-H.50

| | | |
|---|---|---|
| SEQ ID NO: | VH + VL | QVQLVQSGAEVKKPGSSVKVSCKASGYSFTTYYIHWV |
| 1138 | | RQAPGQGLEWMGRIFPGSGNIKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGINVVWHQQKPGKAPKALIY |
| | | SSSHRYSGVPSRFSGSGSGTEFTLTISSLQPEDFATYFCQ |
| | | QFKSYPLTFGQGTKLEIK |
| SEQ ID NO: | VH | QVQLVQSGAEVKKPGSSVKVSCKASGYSFTTYYIHWV |
| 1139 | | RQAPGQGLEWMGRIFPGSGNIKYNEKFKGRVTITADTS |
| | | TSTAYMELSSLRSEDTAVYYCAGSYYSYDVLDYWGQG |
| | | TTVTVSS |

A-H.51

| | | |
|---|---|---|
| SEQ ID NO: | VH + VL | QVQLVQSGAEVKKPGSSVKVSCKASGYSFTTYYIHWV |
| 1140 | | RQAPGQGLEWMGWFFPGSGNIKYNEKFKGRVTITADT |
| | | STSTAYMELSSLRSEDTAVYYCAGSIYSAGVLDYWGQG |
| | | TTVTVSSGGGGSGGGGGGGGSGGGGSDIQMTQSPSFL |
| | | SASVGDRVTITCKASQNVGINVVWHQQKPGKAPKALIY |
| | | SSSHRYSGVPSRFSGSGSGTEFTLTISSLQPEDFATYFCQ |
| | | QFKSYPLTFGQGTKLEIK |
| SEQ ID NO: | VH | QVQLVQSGAEVKKPGSSVKVSCKASGYSFTTYYIHWV |
| 1141 | | RQAPGQGLEWMGWFFPGSGNIKYNEKFKGRVTITADT |
| | | STSTAYMELSSLRSEDTAVYYCAGSIYSAGVLDYWGQG |
| | | TTVTVSS |

A-H.52

| | | |
|---|---|---|
| SEQ ID NO: | VH + VL | QVQLVQSGAEVKKPGSSVKVSCKASGYSFTLGYIHWV |
| 1142 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTLGYIHWV |
| 1143 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |

A-H.53

| | | |
|---|---|---|
| SEQ ID NO: | VH + VL | QVQLVQSGAEVKKPGSSVKVSCKASGYSFRLTYIHWV |
| 1144 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFRLTYIHWV |
| 1145 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |

A-H.54

| | | |
|---|---|---|
| SEQ ID NO: | VH + VL | QVQLVQSGAEVKKPGSSVKVSCKASGYSFHNWYIHWV |
| 1146 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFHNWYIHWV |
| 1147 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |

A-H.55 antibody

| | | |
|---|---|---|
| SEQ ID NO: | HC CDR1 | GYSFTTYYIH |
| 1003 | (Combined) | |
| SEQ ID NO: | HC CDR2 | WFFPGSGNIKYNEKFKG |
| 1004 | (Combined) | |
| SEQ ID NO: | HC CDR3 | SYYSYDVLDY |
| 1005 | (Combined) | |
| SEQ ID NO: | HC CDR1 (Kabat) | TYYIH |
| 10045 | | |
| SEQ ID NO: | HC CDR2 (Kabat) | WFFPGSGNIKYNEKFKG |
| 1004 | | |
| SEQ ID NO: | HC CDR3 (Kabat) | SYYSYDVLDY |
| 1005 | | |
| SEQ ID NO: | HC CDR1 (Chothia) | GYSFTTY |
| 1015 | | |
| SEQ ID NO: | HC CDR2 (Chothia) | FPGSGN |
| 1016 | | |
| SEQ ID NO: | HC CDR3 (Chothia) | SYYSYDVLDY |
| 1005 | | |
| SEQ ID NO: | VH | QVQLVQSGAEVKKPGSSVKVSCKASGYSFTTYYIHWV |
| 1009 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |
| SEQ ID NO: | LC CDR1 | KASQNVGINVV |
| 1006 | (Combined) | |
| SEQ ID NO: | LC CDR2 | SSSHRYS |
| 1007 | (Combined) | |
| SEQ ID NO: | LC CDR3 | QQFKSYPLT |
| 1008 | (Combined) | |
| SEQ ID NO: | LC CDR1 (Kabat) | KASQNVGINVV |
| 1006 | | |
| SEQ ID NO: | LC CDR2 (Kabat) | SSSHRYS |
| 1007 | | |
| SEQ ID NO: | LC CDR3 (Kabat) | QQFKSYPLT |
| 1008 | | |
| SEQ ID NO: | LC CDR1 (Chothia) | KASQNVGINVV |
| 1006 | | |
| SEQ ID NO: | LC CDR2 (chothia) | SSSHRYS |
| 1007 | | |
| SEQ ID NO: | LC CDR3 (chothia) | QQFKSYPLT |
| 1008 | | |
| SEQ ID NO: | VL | QSVLTQPPSVSEAPRQRVTISCKASQNVGINVVWHQQL |
| 1148 | | PGKAPKALIYSSSHRYSGVSDRFSGSGSGTSFSLAISGLQ |
| | | SEDEADYFCQQFKSYPLTFGTGTKVTVL |

A-H.56

| | | |
|---|---|---|
| SEQ ID NO: | VH + VL (ScFv) | QVQLVQSGAEVKKPGSSVKVSCKASGHDFDKFYIHWV |
| 1149 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGNRVAWYQQKPGKAPKAL |
| | | IYSSSHRYKGVPSRFSGSGSGTEFTLTISSLQPEDFATYFC |
| | | QQFKSYPLTFGQGTKLEIK |

A-H.57

| | | |
|---|---|---|
| SEQ ID NO: | VH + VL (ScFv) | QVQLVQSGAEVKKPGSSVKVSCKASGHDFRLTYIHWV |
| 1150 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGDRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.58

| | | |
|---|---|---|
| SEQ ID NO: | VH + VL (ScFv) | QVQLVQSGAEVKKPGSSVKVSCKASGHDFRLTYIHWV |
| 1151 | | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGNRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.59

| | | |
|---|---|---|
| SEQ ID NO: | VH + VL (ScFv) | QVQLVQSGAEVKKPGSSVKVSCKASGHDFRLTYIHWV |
| 1152 | | RQAPGQGLEWMGRIYAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.60

| | | |
|---|---|---|
| SEQ ID NO: | VH + VL (ScFv) | QVQLVQSGAEVKKPGSSVKVSCKASGHDFKLTYIHWV |
| 1153 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGDRVAWHQQKPGKAPKAL |
| | | IYSSSHRYKGVPSRFSGSGSGTEFTLTISSLQPEDFATYFC |
| | | QQFKSYPLTFGQGTKLEIK |

A-H.61

| | | |
|---|---|---|
| SEQ ID NO: | VH + VL (ScFv) | QVQLVQSGAEVKKPGSSVKVSCKASGHDFKLTYIHWV |
| 1154 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVDNRVAWHQQKPGKAPKAL |
| | | IYSSSHRYKGVPSRFSGSGSGTEFTLTISSLQPEDFATYFC |
| | | QQFKSYPLTFGQGTKLEIK |

A-H.62

| | | |
|---|---|---|
| SEQ ID NO: | VH + VL (ScFv) | QVQLVQSGAEVKKPGSSVKVSCKASGHDFRLTYIHWV |
| 1155 | | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.63

| | | |
|---|---|---|
| SEQ ID NO: | VH + VL (ScFv) | QVQLVQSGAEVKKPGSSVKVSCKASGHDFRLTYIHWV |
| 1156 | | RQAPGQGLEWMGRVYAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVEDRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.64

| | | |
|---|---|---|
| SEQ ID NO: | VH + VL (ScFv) | QVQLVQSGAEVKKPGSSVKVSCKASGHDFKLTYIHWV |
| 1157 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.65

| | | |
|---|---|---|
| SEQ ID NO: | VH + VL (ScFv) | QVQLVQSGAEVKKPGSSVKVSCKASGHDFKLTYIHWV |
| 1158 | | RQAPGQGLEWMGRISAGSGNTKYNEKFKGRVTITADTS |
| | | TSTAYMELSSLRSEDTAVYYCAVSYYSYDVLDYWGQG |
| | | TTVTVSSGGGGSGGGGSGGGGSGGGGSDIQMTQSPSFL |
| | | SASVGDRVTITCKASQNVGDRVVWHQQKPGKAPKALI |
| | | YSSSHRYKGVPSRFSGSGSGTEFTLTISSLQPEDFATYFC |
| | | QQFKSYPLTFGQGTKLEIK |

A-H.66

| | | |
|---|---|---|
| SEQ ID NO: | VH + VL (ScFv) | QVQLVQSGAEVKKPGSSVKVSCKASGHDFKLTYIHWV |
| 1159 | | RQAPGQGLEWMGRIYAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGDRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.67

| | | |
|---|---|---|
| SEQ ID NO: | VH + VL (ScFv) | QVQLVQSGAEVKKPGSSVKVSCKASGTDFKLTYIHWV |
| 1160 | | RQAPGQGLEWMGRIFPGSGNVKYNEKFKGRVTITADTS |
| | | TSTAYMELSSLRSEDTAVYYCAVSYYSYDVLDYWGQG |
| | | TTVTVSSGGGGSGGGGSGGGGSGGGGSDIQMTQSPSFL |
| | | SASVGDRVTITCKASQNVDNRVAWYQQKPGKAPKALI |
| | | YSSSHRYKGVPSRFSGSGSGTEFTLTISSLQPEDFATYFC |
| | | QQFKSYPLTFGQGTKLEIK |

A-H.68

| | | |
|---|---|---|
| SEQ ID NO: | VH + VL (ScFv) | QVQLVQSGAEVKKPGSSVKVSCKASGHDFRLTYIHWV |
| 1161 | | RQAPGQGLEWMGRISAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVAWYQQKPGKAPKAL |
| | | IYSSSHRYKGVPSRFSGSGSGTEFTLTISSLQPEDFATYFC |
| | | QQFKSYPLTFGQGTKLEIK |

A-H.69 (also referred to as A-H.13)

| | | |
|---|---|---|
| SEQ ID NO: | VH + VL (ScFv) | QVQLVQSGAEVKKPGSSVKVSCKASGTDFKLTYIHWV |
| 1060 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDNRVAWYQQKPGKAPKALI |
| | | YSSSHRYKGVPSRFSGSGSGTEFTLTISSLQPEDFATYFC |
| | | QQFKSYPLTFGQGTKLEIK |

A-H humanized-matured VH

| | | |
|---|---|---|
| SEQ ID NO: | VH-humanized | QVQLVQSGAEVKKPGSSVKVSCKASGTDFKLTYIHWV |
| 1036 | matured 1 | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |
| SEQ ID NO: | VH-humanized | QVQLVQSGAEVKKPGSSVKVSCKASGTDFKLTYIHWV |
| 1162 | matured 2 | RQAPGQGLEWMGRIFPGSGNVKYNEKFKGRVTITADTS |
| | | TSTAYMELSSLRSEDTAVYYCAVSYYSYDVLDYWGQG |
| | | TTVTVSS |
| SEQ ID NO: | VH-humanized | QVQLVQSGAEVKKPGSSVKVSCKASGHDFRLTYIHWV |
| 1163 | matured 3 | RQAPGQGLEWMGRISAGSGNVKYNEKFKGRVTITADT |
| | | STSTAYMELSSLRSEDTAVYYCAVSYYSYDVLDYWGQ |
| | | GTTVTVSS |

A-H humanized-matured VL

| | | |
|---|---|---|
| SEQ ID NO: | VL-humanized | DIQMTQSPSFLSASVGDRVTITCKASQNVDNRVAWYQQ |
| 1032 | matured 1 | KPGKAPKALIYSSSHRYKGVPSRFSGSGSGTEFTLTISSL |
| | | QPEDFATYFCQQFKSYPLTFGQGTKLEIK |
| SEQ ID NO: | VL-humanized | DIQMTQSPSFLSASVGDRVTITCKASQNVADRVAWYQQ |
| 1102 | matured 2 | KPGKAPKALIYSSSHRYKGVPSRFSGSGSGTEFTLTISSL |
| | | QPEDFATYFCQQFKSYPLTFGQGTKLEIK |

A-H.70

| | | |
|---|---|---|
| SEQ ID NO: | HC CDR1 | GHDFRLTYIH |
| 1164 | | |
| SEQ ID NO: | HC CDR2 | RVSAGSGNVKYNEKFKG |
| 1165 | | |
| SEQ ID NO: | HC CDR3 | SYYSYDVLDY |
| 1005 | | |
| SEQ ID NO: | LC CDR1 | KASQNVGNRVV |
| 1166 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDERLTYIHWV |
| 1168 | (CDRs underlined) | RQAPGQGLEWMGRVSAGSGNVKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGNRVVWHQQ |
| 1169 | (CDRs underlined) | KPGKAPKALIYSSSHRYKGVPSRFSGSGSGTEFTLTISSL |
| | | QPEDFATYFCQQFKSYPLTFGQGTKLEIK |

A-H.71

| | | |
|---|---|---|
| SEQ ID NO: | HC CDR1 | GHDFRLTYIH |
| 1164 | | |
| SEQ ID NO: | HC CDR2 | RIYAGSGNVKYNEKFKG |
| 1170 | | |
| SEQ ID NO: | HC CDR3 | SYYSYDVLDY |
| 1005 | | |
| SEQ ID NO: | LC CDR1 | KASQNVADRVV |
| 1171 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFRLTYIHWV |
| 1172 | (CDRs underlined) | RQAPGQGLEWMGRIYAGSGNVKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVADRVVWHQQ |
| 1173 | (CDRs underlined) | KPGKAPKALIYSSSHRYKGVPSRFSGSGSGTEFTLTISSL |
| | | QPEDFATYFCQQFKSYPLTFGQGTKLEIK |

A-H.72

| | | |
|---|---|---|
| SEQ ID NO: | HC CDR1 | GHDFKLTYIH |
| 1174 | | |
| SEQ ID NO: | HC CDR2 | RVSAGSGNTKYNEKFKG |
| 1175 | | |
| SEQ ID NO: | HC CDR3 | SYYSYDVLDY |
| 1005 | | |
| SEQ ID NO: | LC CDR1 | KASQNVGDRVA |
| 1313 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1177 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGDRVAWHQQ |
| 1096 | (CDRs underlined) | KPGKAPKALIYSSSHRYKGVPSRFSGSGSGTEFTLTISSL |
| | | QPEDFATYFCQQFKSYPLTFGQGTKLEIK |

A-H.73

| | | |
|---|---|---|
| SEQ ID NO: | HC CDR1 | GHDFKLTYIH |
| 1174 | | |
| SEQ ID NO: | HC CDR2 | RVSAGSGNTKYNEKFKG |
| 1175 | | |
| SEQ ID NO: | HC CDR3 | SYYSYDVLDY |
| 1005 | | |
| SEQ ID NO: | LC CDR1 | KASQNVDNRVA |
| 1176 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1177 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVDNRVAWHQQ |
| 1122 | (CDRs underlined) | KPGKAPKALIYSSSHRYKGVPSRFSGSGSGTEFTLTISSL |
| | | QPEDFATYFCQQFKSYPLTFGQGTKLEIK |

A-H.74

| | | |
|---|---|---|
| SEQ ID NO: | HC CDR1 | GHDFRLTYIH |
| 1164 | | |
| SEQ ID NO: | HC CDR2 | RVSAGSGNVKYNEKFKG |
| 1165 | | |
| SEQ ID NO: | HC CDR3 | SYYSYDVLDY |
| 1005 | | |
| SEQ ID NO: | LC CDR1 | KASQNVADRVV |
| 1171 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VE | QVQLVQSGAEVKKPGSSVKVSCKASGHDERLTYIHWV |
| 1168 | (CDRs underlined) | RQAPGQGLEWMGRVSAGSGNVKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVADRVVWHQQ |
| 1173 | (CDRs underlined) | KPGKAPKALIYSSSHRYKGVPSRFSGSGSGTEFTLTISSL |
| | | QPEDFATYFCQQFKSYPLTFGQGTKLEIK |

A-H.75

| | | |
|---|---|---|
| SEQ ID NO: | HC CDR1 | GHDFRLTYIH |
| 1164 | | |
| SEQ ID NO: | HC CDR2 | RVYAGSGNTKYNEKFKG |
| 1178 | | |
| SEQ ID NO: | HC CDR3 | SYYSYDVLDY |
| 1005 | | |
| SEQ ID NO: | LC CDR1 | KASQNVEDRVV |
| 1179 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDERLTYIHWV |
| 1180 | (CDRs underlined) | RQAPGQGLEWMGRVYAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVEDRVVWHQQ |
| 1317 | (CDRs underlined) | KPGKAPKALIYSSSHRYKGVPSRFSGSGSGTEFTLTISSL |
| | | QPEDFATYFCQQFKSYPLTFGQGTKLEIK |

A-H.76

| | | |
|---|---|---|
| SEQ ID NO: | HC CDR1 | GHDFKLTYIH |
| 1174 | | |
| SEQ ID NO: | HC CDR2 | RVSAGSGNTKYNEKFKG |
| 1175 | | |
| SEQ ID NO: | HC CDR3 | SYYSYDVLDY |
| 1005 | | |
| SEQ ID NO: | LC CDR1 | KASQNVADRVV |
| 1171 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1177 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVADRVVWHQQ |
| 1173 | (CDRs underlined) | KPGKAPKALIYSSSHRYKGVPSRFSGSGSGTEFTLTISSL |
| | | QPEDFATYFCQQFKSYPLTFGQGTKLEIK |

A-H.77

| | | |
|---|---|---|
| SEQ ID NO: | HC CDR1 | GHDFKLTYIH |
| 1174 | | |
| SEQ ID NO: | HC CDR2 | RISAGSGNTKYNEKFKG |
| 1181 | | |
| SEQ ID NO: | HC CDR3 | SYYSYDVLDY |
| 1005 | | |
| SEQ ID NO: | LC CDR1 | KASQNVGDRVV |
| 1182 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1183 | (CDRs underlined) | RQAPGQGLEWMGRISAGSGNTKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVGDRVVWHQQ |
| 1184 | (CDRs underlined) | KPGKAPKALIYSSSHRYKGVPSRFSGSGSGTEFTLTISSL |
| | | QPEDFATYFCQQFKSYPLTFGQGTKLEIK |

A-H.78

| | | |
|---|---|---|
| SEQ ID NO: | HC CDR1 | GHDFKLTYIH |
| 1174 | | |
| SEQ ID NO: | HC CDR2 | RIYAGSGNTKYNEKFKG |
| 1314 | | |
| SEQ ID NO: | HC CDR3 | SYYSYDVLDY |
| 1005 | | |
| SEQ ID NO: | LC CDR1 | KASQNVGDRVV |
| 1182 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1185 | (CDRs underlined) | RQAPGQGLEWMGRIYAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGDRVVWHQQ |
| 1184 | (CDRs underlined) | KPGKAPKALIYSSSHRYKGVPSRFSGSGSGTEFTLTISSL |
| | | QPEDFATYFCQQFKSYPLTFGQGTKLEIK |

A-H.79

| | | |
|---|---|---|
| SEQ ID NO: | HC CDR1 | GHDFKLTYIH |
| 1174 | | |
| SEQ ID NO: | HC CDR2 | RVSAGSGNTKYNEKFKG |
| 1175 | | |
| SEQ ID NO: | HC CDR3 | SYYSYDVLDY |
| 1005 | | |
| SEQ ID NO: | LC CDR1 | RASQNVDNRLG |
| 1186 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1177 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCRASQNVDNRLGWHQQ |
| 1187 | (CDRs underlined) | KPGKAPKALIYSSSHRYKGVPSRFSGSGSGTEFTLTISSL |
| | | QPEDFATYFCQQFKSYPLTFGQGTKLEIK |

A-H.80

| | | |
|---|---|---|
| SEQ ID NO: | HC CDR1 | GHDFKLTYIH |
| 1174 | | |
| SEQ ID NO: | HC CDR2 | RVSAGSGNTKYNEKFKG |
| 1175 | | |
| SEQ ID NO: | HC CDR3 | SYYSYDVLDY |
| 1005 | | |
| SEQ ID NO: | LC CDR1 | KASQNVDNRVA |
| 1176 | | |
| SEQ ID NO: | LC CDR2 | AASSLQK |
| 1188 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1177 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVDNRVAWHQQ |
| 1189 | (CDRs underlined) | KPGKAPKALIYAASSLQKGVPSRFSGSGSGTEFTLTISSL |
| | | QPEDFATYFCQQFKSYPLTFGQGTKLEIK |

A-H.81

| | | |
|---|---|---|
| SEQ ID NO: | HC CDR1 | GHDFKLTYIH |
| 1174 | | |
| SEQ ID NO: | HC CDR2 | RVSAGSGNTKYNEKFKG |
| 1175 | | |
| SEQ ID NO: | HC CDR3 | SYYSYDVLDY |
| 1005 | | |
| SEQ ID NO: | LC CDR1 | KASQNVDNRVA |
| 1176 | | |
| SEQ ID NO: | LC CDR2 | SSSHRYK |
| 1167 | | |
| SEQ ID NO: | LC CDR3 | LQHNSYPLT |
| 1190 | | |
| SEQ ID NO: | VH | QVQLVQSGAEVKKPGSSVKVSCKASGHDFKLTYIHWV |
| 1177 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVDNRVAWHQQ |
| 1191 | (CDRs underlined) | KPGKAPKALIYSSSHRYKGVPSRFSGSGSGTEFTLTISSL |
| | | QPEDFATYFCLQHNSYPLTFGQGTKLEIK |

A-H.82

| | | |
|---|---|---|
| SEQ ID NO: | HC CDR1 | GHDFKLTYIH |
| 1174 | | |
| SEQ ID NO: | HC CDR2 | RVSAGSGNVNYAQKFQG |
| 1192 | | |
| SEQ ID NO: | HC CDR3 | SYYSYDVLDY |
| 1005 | | |
| SEQ ID NO: | LC CDR1 | RASQNVDNRLG |
| 1186 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1193 | (CDRs underlined) | RQAPGQGLEWMGRVSAGSGNVNYAQKFQGRVTITAD |
| | | TSTSTAYMELSSLRSEDTAVYYCAVSYYSYDVLDYWG |
| | | QGTTVTVSS |
| SEQ ID NO: | VL | DIQMTQSPSFLSASVGDRVTITCRASQNVDNRLGWHQQ |
| 1187 | (CDRs underlined) | KPGKAPKALIYSSSHRYKGVPSRFSGSGSGTEFTLTISSL |
| | | QPEDFATYFCQQFKSYPLTFGQGTKLEIK |

A-H.83

| | | |
|---|---|---|
| SEQ ID NO: | HC CDR1 | GHDFKLTYIH |
| 1174 | | |
| SEQ ID NO: | HC CDR2 | RVSAGSGNVNYAQKFQG |
| 1192 | | |
| SEQ ID NO: | HC CDR3 | SYYSYDVLDY |
| 1005 | | |
| SEQ ID NO: | LC CDR1 | KASQNVDNRVA |
| 1176 | | |
| SEQ ID NO: | LC CDR2 | AASSLQK |
| 1188 | | |
| SEQ ID NO: | LC CDR3 | QQFKSYPLT |
| 1008 | | |
| SEQ ID NO: | VH | QVQLVQSGAEVKKPGSSVKVSCKASGHDFKLTYIHWV |
| 1193 | (CDRs underlined) | RQAPGQGLEWMGRVSAGSGNVNYAQKFQGRVTITAD |
| | | TSTSTAYMELSSLRSEDTAVYYCAVSYYSYDVLDYWG |
| | | QGTTVTVSS |
| SEQ ID NO: | VL | DIQMTQSPSFLSASVGDRVTITCKASQNVDNRVAWHQQ |
| 1189 | (CDRs underlined) | KPGKAPKALIYAASSLQKGVPSRFSGSGSGTEFTLTISSL |
| | | QPEDFATYFCQQFKSYPLTFGQGTKLEIK |

A-H.84

| | | |
|---|---|---|
| SEQ ID NO: | HC CDR1 | GHDFKLTYIH |
| 1174 | | |
| SEQ ID NO: | HC CDR2 | RVSAGSGNVNYAQKFQG |
| 1192 | | |
| SEQ ID NO: | HC CDR3 | SYYSYDVLDY |
| 1005 | | |
| SEQ ID NO: | LC CDR1 | KASQNVDNRVA |
| 1176 | | |
| SEQ ID NO: | LC CDR2 | SSSHRYK |
| 1167 | | |
| SEQ ID NO: | LC CDR3 | LQHNSYPLT |
| 1190 | | |
| SEQ ID NO: | VH | QVQLVQSGAEVKKPGSSVKVSCKASGHDFKLTYIHWV |
| 1193 | (CDRs underlined) | RQAPGQGLEWMGRVSAGSGNVNYAQKFQGRVTITAD |
| | | TSTSTAYMELSSLRSEDTAVYYCAVSYYSYDVLDYWG |
| | | QGTTVTVSS |
| SEQ ID NO: | VL | DIQMTQSPSFLSASVGDRVTITCKASQNVDNRVAWHQQ |
| 1191 | (CDRs underlined) | KPGKAPKALIYSSSHRYKGVPSRFSGSGSGTEFTLTISSL |
| | | QPEDFATYFCLQHNSYPLTFGQGTKLEIK |

A-H.85

| | | |
|---|---|---|
| SEQ ID NO: | HC CDR1 | GHDFRLTYIH |
| 1164 | | |
| SEQ ID NO: | HC CDR2 | RVSAGSGNTKYNEKFKG |
| 1175 | | |
| SEQ ID NO: | HC CDR3 | SYYSYDVLDY |
| 1005 | | |
| SEQ ID NO: | LC CDR1 | KASQNVGDRVV |
| 1182 | | |
| SEQ ID NO: | LC CDR2 | SSSHRYK |
| 1167 | | |
| SEQ ID NO: | LC CDR3 | QQFKSYPLT |
| 1008 | | |
| SEQ ID NO: | VH (CDRs | QVQLVQSGAEVKKPGSSVKVSCKASGHDFRLTYIHWV |
| 1194 | underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGDRVVWHQQ |
| 1184 | (CDRs underlined) | KPGKAPKALIYSSSHRYKGVPSRFSGSGSGTEFTLTISSL |
| | | QPEDFATYFCQQFKSYPLTFGQGTKLEIK |

TABLE 22
Exemplary multispecific molecule that comprise a first domain that binds
to a first target molecule, wherein the exemplary target molecule
comprises TCRVα 1 (e.g., TCRVα 1-2). Exemplary anti-TCRVα 1 antibody
includes, but are not limited to, BMX001, which binds to human TCRVα 1
(e.g., TCRVα 1-2). CDRs are underlined.
BMX001
Antibody binding to human TCRVα 1-2

| SEQ ID NO: | VH | QVQLVQSGAEVKKPGSSVKVSCKASGFNIKDTHWVRQ |
|---|---|---|
| 1195 | | APGQGLEWMGTDPASGDTRVTITADTSTSTAYMELSSL |
| | | RSEDTAVYYCAVCAHYYRDDVNYAMDYWGQGTTVT |
| | | VSS |
| SEQ ID NO: | VL | DIQMTQSPSFLSASVGDRVTITCQNVGSNWHQQKPGKA |
| 1196 | | PKALIYSSSGVPSRFSGSGSGTEFTLTISSLQPEDFATYFC |
| | | QQYNTYPYTFGQGTKLEIK |
| SEQ ID NO: | HC CDR1 | GFNIKDTH |
| 1197 | | |
| SEQ ID NO: | HC CDR2 | TDPASGDT |
| 1198 | | |
| SEQ ID NO: | HC CDR3 | CAHYYRDDVNYAMDY |
| 1199 | | |
| SEQ ID NO: | LC CDR1 | QNVGSN |
| 1200 | | |
| SEQ ID NO: | LC CDR1 | SSS |
| 1201 | | |
| SEQ ID NO: | LC CDR1 | QQYNTYPYT |
| 1202 | | |

TABLE 23
Constant region amino acid sequences of human IgG heavy chains and human kappa
light chain

| | | |
|---|---|---|
| Human kappa | LC | RTVAAPSVFI FPPSDEQLKS GTASVVCLLN NFYPREAKVQ |
| constant region | | WKVDNALQSG NSQESVTEQD SKDSTYSLSS TLTLSKADYE |
| SEQ ID NO: | | KHKVYACEVT HQGLSSPVTK SFNRGEC |
| 1039 | | |
| Human | LC | RTVAAPSVFIFPPSDEQLKSGTASVVCLLNNFYPREAKVQWKVDNALQ |
| Immunoglobulin | | SGNSQESVTEQDSKDSTYSLSSTLTLSKADYEKHKVYACEVTHQGLSSP |
| kappa constant | | VTKSFNRGEC |
| C region | | |
| SEQ ID NO: | | |
| 1203 | | |
| IgG4 (S228P) | HC | ASTKGPSVFPLAPCSRSTSESTAALGCLVKDYFPEPVTVSWNSGALTSG |
| mutant constant | | VHTFPAVLQSSGLYSLSSVVTVPSSSLGTKTYTCNVDHKPSNTKVDKR |
| region (EU | | VESKYGPPCPPCPAPEFLGGPSVFLFPPKPKDTLMISRTPEVTCVVVDVS |
| Numbering) | | QEDPEVQFNWYVDGVEVHNAKTKPREEQFNSTYRVVSVLTVLHQDW |
| SEQ ID NO: | | LNGKEYKCKVSNKGLPSSIEKTISKAKGQPREPQVYTLPPSQEEMTKNQ |
| 1040 | | VSLTCLVKGFYPSDIAVEWESNGQPENNYKTTPPVLDSDGSFFLYSRLT |
| | | VDKSRWQEGNVFSCSVMHEALHNHYTQKSLSLSLG |
| IgG1 wild type | HC | ASTKGPSVFPLAPSSKSTSGGTAALGCLVKDYFPEPVTVSWNSGALTSG |
| SEQ ID NO: | | VHTFPAVLQSSGLYSLSSVVTVPSSSLGTQTYICNVNHKPSNTKVDKRV |
| 1041 | | EPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| IgG1 (N297A) | HC | ASTKGPSVFPLAPSSKSTSGGTAALGCLVKDYFPEPVTVSWNSGALTSG |
| mutant constant | | VHTFPAVLQSSGLYSLSSVVTVPSSSLGTQTYICNVNHKPSNTKVDKRV |
| region (EU | | EPKSCDKTHTCPPCPAPELLGGPSVFLFPPKPKDTLMISRTPEVTCVVVD |
| Numbering) | | VSHEDPEVKFNWYVDGVEVHNAKTKPREEQYASTYRVVSVLTVLHQ |
| SEQ ID NO: | | DWLNGKEYKCKVSNKALPAPIEKTISKAKGQPREPQVYTLPPSREEMT |
| 1042 | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| IgM constant | HC | GSASAPTLFPLVSCENSPSDTSSVAVGCLAQDFLPDSITFSWKYKNNSDI |
| delta CDC | | SSTRGFPSVLRGGKYAATSQVLLPSKDVMQGTDEHVVCKVQHPNGNK |
| (P311A, P313S) | | EKNVPLPVIAELPPKVSVFVPPRDGFFGNPRKSKLICQATGFSPRQIQVS |
| SEQ ID NO: | | WLREGKQVGSGVTTDQVQAEAKESGPTTYKVTSTLTIKESDWLGQSM |
| 1073 | | FTCRVDHRGLTFQQNASSMCVPDQDTAIRVFAIPPSFASIFLTKSTKLTC |
| | | LVTDLTTYDSVTISWTRQNGEAVKTHTNISESHPNATFSAVGEASICED |
| | | DWNSGERFTCTVTHTDLASSLKQTISRPKGVALHRPDVYLLPPAREQL |
| | | NLRESATITCLVTGFSPADVFVQWMQRGQPLSPEKYVTSAPMPEPQAP |
| | | GRYFAHSILTVSEEEWNTGETYTCVVAHEALPNRVTERTVDKSTGKPT |
| | | LYNVSLVMSDTAGTCY |
| IgGA1 | HC | ASPTSPKVFPLSLCSTQPDGNVVIACLVQGFFPQEPLSVTWSESGQGVT |
| SEQ ID NO: | | ARNFPPSQDASGDLYTTSSQLTLPATQCLAGKSVTCHVKHYTNPSQDV |
| 1074 | | TVPCPVPSTPPTPSPSTPPTPSPSCCHPRLSLHRPALEDLLLGSEANLTCT |
| | | LTGLRDASGVTFTWTPSSGKSAVQGPPERDLCGCYSVSSVLPGCAEPW |
| | | NHGKTFTCTAAYPESKTPLTATLSKSGNTFRPEVHLLPPPSEELALNELV |
| | | TLTCLARGFSPKDVLVRWLQGSQELPREKYLTWASRQEPSQGTTTFAV |
| | | TSILRVAAEDWKKGDTFSCMVGHEALPLAFTQKTIDRLAGKPTHVNVS |
| | | VVMAEVDGTCY |
| IgGA2 | HC | ASPTSPKVFPLSLDSTPQDGNVVVACLVQGFFPQEPLSVTWSESGQNVT |
| SEQ ID NO: | | ARNFPPSQDASGDLYTTSSQLTLPATQCPDGKSVTCHVKHYTNSSQDV |
| 1075 | | TVPCRVPPPPPCCHPRLSLHRPALEDLLLGSEANLTCTLTGLRDASGATF |
| | | TWTPSSGKSAVQGPPERDLCGCYSVSSVLPGCAQPWNHGETFTCTAAH |
| | | PELKTPLTANITKSGNTFRPEVHLLPPPSEELALNELVTLTCLARGFSPK |
| | | DVLVRWLQGSQELPREKYLTWASRQEPSQGTTTYAVTSILRVAAEDW |
| | | KKGETFSCMVGHEALPLAFTQKTIDRMAGKPTHINVSVVMAEADGTC |
| | | Y |
| Human Ig_J | HC | MKNHLLFWGVLAVFIKAVHVKAQEDERIVLVDNKCKCARITSRIIRSSE |
| chain | | DPNEDIVERNIRIIVPLNNRENISDPTSPLRTRFVYHLSDLCKKCDPTEVE |
| SEQ ID NO: | | LDNQIVTATQSNICDEDSATETCYTYDRNKCYTAVVPLVYGGETKMVE |
| 1076 | | TALTPDACYPD |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKKV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYASTYRVVSVLTVLHQ |
| 1204 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKKV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYASTYRVVSVLTVLHQ |
| 1205 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGKEPEA |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKRV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYNSTYRVVSVLTVLHQ |
| 1041 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| Human Fc Knob | HC | DKTHTCPPCPAPELLGGPSVFLFPPKPKDTLMISRTPEVTCVVVDVSHE |
| cys N297A | | DPEVKFNWYVDGVEVHNAKTKPREEQYASTYRVVSVLTVLHQDWLN |
| SEQ ID NO: | | GKEYKCKVSNKALPAPIEKTISKAKGQPREPQVYTLPPCREEMTKNQV |
| 1206 | | SLWCLVKGFYPSDIAVEWESNGQPENNYKTTPPVLDSDGSFFLYSKLT |
| | | VDKSRWQQGNVFSCSVMHEALHNHYTQKSLSLSPGK |
| Human IgG1 | HC | ASTKGPSVFPLAPSSKSTSGGTAALGCLVKDYFPEPVTVSWNSGALTSG |
| hole cys N297A | | VHTFPAVLQSSGLYSLSSVVTVPSSSLGTQTYICNVNHKPSNTKVDKRV |
| SEQ ID NO: | | EPKSCDKTHTCPPCPAPELLGGPSVFLFPPKPKDTLMISRTPEVTCVVVD |
| 1207 | | VSHEDPEVKFNWYVDGVEVHNAKTKPREEQYASTYRVVSVLTVLHQ |
| | | DWLNGKEYKCKVSNKALPAPIEKTISKAKGQPREPQVCTLPPSREEMT |
| | | KNQVSLSCAVKGFYPSDIAVEWESNGQPENNYKTTPPVLDSDGSFFLV |
| | | SKLTVDKSRWQQGNVFSCSVMHEALHNHYTQKSLSLSPGK |

TABLE 24
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to
form a stabilizing disulfide bridge

| Position | Knob CH3 | Hole CH3 |
|---|---|---|
| S354 | S354C | — |
| Y349 | — | Y349C |

TABLE 25
CRS grading

| | |
|---|---|
| Gr1 | Supportive care only |
| Gr2 | IV therapies +/− hospitalization. |
| Gr3 | Hypotension requiring IV fluids or low-dose |
| | vasoactives or hypoxemia requiring oxygen, |
| | CPAP, or BIPAP. |
| Gr4 | Hypotension requiring high-dose vasoactives or |
| | hypoxemia requiring mechanical ventilation. |
| Gr 5 | Death |

TABLE 26
CTCAE v 4.0 CRS grading scale

| CRS | |
|---|---|
| grade | Characteristics |
| Grade 1 | Mild; No infusion interruption; No intervention |
| Grade 2 | Infusion interruption indicated but responds promptly to symptomatic treatment (e.g., |
| | antihistamines, NSAIDS, narcotics, IV fluids); prophylactic medications indicated |
| | for <=24 hrs |
| Grade 3 | Prolonged (e.g., not rapidly responsive to symptomatic medications and/or brief |
| | interruption of infusion); recurrence of symptoms following initial improvement; |
| | hospitalization indicated for clinical sequelae (e.g., renal impairment, pulmonary |
| | infiltrates) |
| Grade 4 | Life threatening consequences; pressor or ventilator support |

TABLE 27
NCI CRS grading scale

| CRS | |
|---|---|
| grade | Characteristics |
| Grade 1 | Symptoms are not life threatening and require symptomatic treatment only; e.g., fever, |
| | nausea, fatigue, headache, myalgias, malaise |
| Grade 2 | Symptoms require and respond to moderate intervention; Oxygen requirement <40% or |
| | hypotension responsive to fluids or low dose pressors or Grade 2 organ toxicity |
| Grade 3 | Symptoms require and respond to aggressive intervention; Oxygen requirement >=40% or |
| | Hypotension requiring high dose or multiple pressors or grade 3 organ toxicity or grade 4 |
| | transaminitis |
| Grade 4 | Life threatening symptoms Requirement for ventilator support or Grade 4; organ toxicity |
| | (excluding transaminitis) |

TABLE 28
List of TCRβV subfamilies and subfamily members

| Subfamily | Subfamily members |
|---|---|
| TCRβ V4 | TCRβ V4-2*01, TCRβ V4-3*01, or TCRβ V4-1*01 |
| TCRβ V6 | TCRβ V6-4*01, TCRβ V6-4*02, |
| Also | TCRβ V6-9*01, TCRβ V6-8*01, |
| referred | TCRβ V6-5*01, TCRβ V6-6*02, |
| to as: | TCRβ V6-6*01, TCRβ V6-2*01, |
| TCR VB | TCRβ V6-3*01 or TCRβ V6-1*01. |
| 13.1 | |

TABLE 29
Exemplary anti-TCRβV antibody molecules

| TCRvβ | TCRvβ | Reagents monoclonal antibodies | | |
|---|---|---|---|---|
| gene | allele | Clone name and | | |
| name | name | Specificity | Company product | Isotype |
| TCRvβ4-1 | TCRvβ4- | ZOE (TCRvβ4-1, | Serotec V BETA 7 | Mouse |
| | 1*01 | TCRvβ4-2, TCRvβ4-3) | Coulter Vbeta7 | IgG2a |
| | TCRvβ4- | 3G5 (TCRvβ4-1) | Pierce Endogen V beta 7.1 | Mouse |
| | 1*02 | | | IgG2b |
| TCRvβ4-2 | TCRvβ4- | ZOE (TCRvβ4-1, | Serotec V BETA 7 | Mouse |
| | 2*01 | TCRvβ4-2, TCRvβ4-3) | Coulter Vbeta7 | IgG2a |
| | TCRvβ4- | | | |
| | 2*02 | | | |
| TCRvβ4-3 | TCRvβ4- | ZOE (TCRvβ4-1, | Serotec V BETA 7 | Mouse |
| | 3*01 | TCRvβ4-2, TCRvβ-3) | Coulter Vbeta7 | IgG2a |
| | TCRvβ4- | | | |
| | 3*02 | | | |
| | TCRvβ4- | | | |
| | 3*03 | | | |
| | TCRvβ4- | ZIZOU4 (TCRvβ4-3) | Coulter Vbeta7.2 | Mouse |
| | 3*04 | | | IgG2a |
| | TCRvβ5- | | | |
| | 8*02 | | | |
| TCRvβ6-1 | TCRvβ6- | BAM13 (TCRvβ6-1, | Pierce Endogen V beta 13 | Mouse |
| | 1*01 | TCRvβ6-5) | BD Biosciences Vbeta13.1, | IgG1 |
| | | | 13.3 | |
| TCRvβ6-2 | TCRVβ6- | H132 | Coulter Vbeta13.2 | Mouse |
| | 2*01 | | | IgG1 |
| TCRvβ6-3 | TCRVβ6- | | | |
| | 3*01 | | | |
| TCRvβ6-4 | TCRVβ6- | | | |
| | 4*01 | | | |
| | TCRvβ6- | | | |
| | 4*02 | | | |
| TCRvβ6-5 | TCRvβ6- | IMMU 222 (TCRvβ6-5, | Serotec V BETA 13.1 | Mouse |
| | 5*01 | TCRvβ6-6 and TCRvβ6- | Coulter Vbeta13.1 | IgG2b |
| | | 9) | Pierce Endogen V beta 13 | Mouse |
| | | BAM13 (TCRvβ6-1, | BD Biosciences Vbeta13.1, | IgG1 |
| | | TCRvβ6-5) | 13.3 | |
| TCRvβ6-6 | TCRvβ6- | JU-74 (TCRvβ6-6) | Serotec Vbeta13.6 | Mouse |
| | 6*01 | JU74.3 (TCRvβ6-6) | Coulter Vbeta13.6 | IgG1 |
| | TCRvβ6- | IMMU 222 (TCRvβ6-5, | | |
| | 6*02 | TCRvβ6-6 and TCRvβ6- | | |
| | TCRvβ6- | 9) | | |
| | 6*03 | | | |
| | TCRvβ6- | | Serotec V BETA 13.1 | Mouse |
| | 6*04 | | Coulter Vbeta13.1 | IgG2b |
| | TCRvβ6- | | | |
| | 6*05 | | | |
| TCRvβ6-8 | TCRvβ6- | | | |
| | 8*01 | | | |
| TCRvβ6-9 | TCRvβ6- | IMMU 222 (TCRvβ6-5, | Serotec V BETA 13.1 | Mouse |
| | 9*01 | TCRvβ6-6 and TCRvβ6- | Coulter Vbeta13.1 | IgG2b |
| | | 9) | | |

TABLE 30
Amino acid sequences for anti TCRβ V4 antibodies. Amino acid and nucleotide
sequences for murine and humanized antibody molecules which bind to TCRBV 4 (e.g.,
TCRBV 4-1, TCRBV 4-2 or TCRBV 4-3). The amino acid the heavy and light chain CDRs,
and the amino acid and nucleotide sequences of the heavy and light chain variable
regions, and the heavy and light chains are shown.
ZOE (murine), also referred to as BJ1538 or as Antibody L
Binds to human TCRVβ 4-1,4-2,4-3

| | | |
|---|---|---|
| SEQ ID NO: | HC CDR1 (Kabat) | DYYMY |
| 1208 | | |
| SEQ ID NO: | HC CDR2 (Kabat) | TISGGGSYTYSPDSVKG |
| 1209 | | |
| SEQ ID NO: | HC CDR3 (Kabat) | ERDIYYGNFNAMVY |
| 1210 | | |
| SEQ ID NO: | HC CDR1 (Chothia) | GFTFSDY |
| 1211 | | |
| SEQ ID NO: | HC CDR2 (Chothia) | SGGGSY |
| 1212 | | |
| SEQ ID NO: | HC CDR3 (Chothia) | ERDIYYGNFNAMVY |
| 1210 | | |
| SEQ ID NO: | HC CDR1 | GFTFSDYYMY |
| 1213 | (Combined) | |
| SEQ ID NO: | HC CDR2 | TISGGGSYTYSPDSVKG |
| 1209 | (Combined) | |
| SEQ ID NO: | HC | ERDIYYGNFNAMVY |
| 1210 | CDR3(Combined) | |
| SEQ ID NO: | LC CDR1 (Kabat) | RASKSVSTSGYSYMH |
| 1214 | | |
| SEQ ID NO: | LC CDR2 (Kabat) | LASNLES |
| 1215 | | |
| SEQ ID NO: | LC CDR3 (Kabat) | QHSRDLPWT |
| 1216 | | |
| SEQ ID NO: | LC CDR1 (Chothia) | SKSVSTSGYSY |
| 1217 | | |
| SEQ ID NO: | LC CDR2 (Chothia) | LASNLES |
| 1215 | | |
| SEQ ID NO: | LC CDR3 (Chothia) | QHSRDLPWT |
| 1216 | | |
| SEQ ID NO: | LC CDR1 | RASKSVSTSGYSYMH |
| 1214 | (Combined) | |
| SEQ ID NO: | LC CDR2 | LASNLES |
| 1215 | (Combined) | |
| SEQ ID NO: | LC | QHSRDLPWT |
| 1216 | CDR3(Combined) | |
| SEQ ID NO: | VL | DIVLTQSPVSLTVSLGQRATISCRASKSVSTSGYSYMHW |
| 1218 | | YQQKPGQPPKLLIYLASNLESGVPARFSGSGSGTDFTLNI |
| | | HPVEEEDAATYYCQHSRDLPWTFGGGTKLEIK |
| SEQ ID NO: | VH | EVQLVESGGGLVKPGGSLKLSCAASGFTFSDYYMYWVR |
| 1219 | | QTPEKRLEWVATISGGGSYTYSPDSVKGRFTISRDNAKN |
| | | NLYLQMSSLRSEDTAMYFCARERDIYYGNFNAMVYWG |
| | | RGTSVTVSS |

VH for ZOE (humanized) also referred to as Antibody L-H
Binds to human TCRVβ 4-1,4-2,4-3

| | | |
|---|---|---|
| SEQ ID NO: | VH-1 | EVQLLESGGGLVQPGGSLRLSCAASGFTFSDYYMYWVR |
| 1220 | | QAPGKGLEWVATISGGGSYTYSPDSVKGRFTISRDNSKN |
| | | TLYLQMNSLRAEDTAVYYCARERDIYYGNFNAMVYWG |
| | | RGTLVTVSS |
| SEQ ID NO: | VH-2 | EQLVESGGGLVQPGGSLRLSCAASGFTFSDYYMYWVR |
| 1221 | | QAPGKGLEWVATISGGGSYTYSPDSVKGRFTISRDNAKN |
| | | SLYLQMNSLRAEDTAVYYCARERDIYYGNFNAMVYWG |
| | | RGTLVTVSS |
| SEQ ID NO: | VH-3 | QVQLVESGGGVVQPGRSLRLSCAASGFTFSDYYMYWV |
| 1222 | | RQAPGKGLEWVATISGGGSYTYSPDSVKGRFTISRDNS |
| | | KNTLYLQMNSLRAEDTAVYYCARERDIYYGNFNAMV |
| | | YWGRGTLVTVSS |
| SEQ ID NO: | VH-4 | QVQLVESGGGLVKPGGSLRLSCAASGFTFSDYYMYWIR |
| 1223 | | QAPGKGLEWVATISGGGSYTYSPDSVKGRFTISRDNAKN |
| | | SLYLQMNSLRAEDTAVYYCARERDIYYGNFNAMVYWG |
| | | RGTLVTVSS |

VL for ZOE (humanized) also referred to as Antibody L-H
Binds to human TCRVβ 4-1,4-2,4-3

| | | |
|---|---|---|
| SEQ ID NO: | VL-1 | EIVLTQSPGTLSLSPGERATLSCRASKSVSTSGYSYMHW |
| 1224 | | YQQKPGQAPRLLIYLASNLESGIPDRFSGSGSGTDFTLTIS |
| | | RLEPEDFAVYYCQHSRDLPWTFGGGTKVEIK |
| SEQ ID NO: | VL-2 | EIVLTQSPATLSLSPGERATLSCRASKSVSTSGYSYMHW |
| 1225 | | YQQKPGQAPRLLIYLASNLESGIPARFSGSGSGTDFTLTIS |
| | | SLEPEDFAVYYCQHSRDLPWTFGGGTKVEIK |
| SEQ ID NO: | VL-3 | DIQLTQSPSTLSASVGDRVTITCRASKSVSTSGYSYMHW |
| 1226 | | YQQKPGKAPKLLIYLASNLESGVPSRFSGSGSGTEFTLTIS |
| | | SLQPDDFATYYCQHSRDLPWTFGGGTKVEIK |
| SEQ ID NO: | VL-4 | AIQLTQSPSSLSASVGDRVTITCRASKSVSTSGYSYMHW |
| 1227 | | YQQKPGKAPKLLIYLASNLESGVPSRFSGSGSGTDFTLTI |
| | | SSLQPEDFATYYCQHSRDLPWTFGGGTKVEIK |

TABLE 31
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

TABLE 32
Exemplary Construct Sequences
TCRVβ/IL15-sushi bispecific

| | SEQ | | |
|---|---|---|---|
| | ID | | |
| Chain | NO: | Component | Sequence |
| Chain 1: | 1228 | Full Sequence | QVQLVQSGAEVKKPGSSVKVSCKASGHDFRLTYIHWVRQAPGQG |
| TCRVβ- | | | LEWMGRVSAGSGNVKYNEKFKGRVTITADTSTSTAYMELSSLRS |
| Fc_Hole_Cys_ | | | EDTAVYYCAVSYYSYDVLDYWGQGTTVTVSSASTKGPSVFPLAP |
| N297A heavy | | | SSKSTSGGTAALGCLVKDYFPEPVTVSWNSGALTSGVHTFPAVLQ |
| chain (CDRs | | | SSGLYSLSSVVTVPSSSLGTQTYICNVNHKPSNTKVDKRVEPKSCD |
| underlined) | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVCTLPPSR |
| | | | EEMTKNQVSLSCAVKGFYPSDIAVEWESNGQPENNYKTTPPVLDS |
| | | | DGSFFLVSKLTVDKSRWQQGNVFSCSVMHEALHNHYTQKSLSLS |
| | | | PGK |
| | 1168 | anti-TCRvβ VH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRS |
| | | | EDTAVYYCAVSYYSYDVLDYWGQGTTVTVSS |
| | 1164 | HC CDR1 | GHDFRLTYIH |
| | 229 | HC CDR1 | LTYIH |
| | 1234 | HC CDR1 | FRLTYIH |
| | 1165 | HC CDR2 | RVSAGSGNVKYNEKFKG |
| | 1005 | HC CDR3 | SYYSYDVLDY |
| | 1207 | Human IgG1 | ASTKGPSVFPLAPSSKSTSGGTAALGCLVKDYFPEPVTVSWNSGA |
| | | hole cys N297A | LTSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMIS |
| | | | RTPEVTCVVVDVSHEDPEVKFNWYVDGVEVHNAKTKPREEQYA |
| | | | STYRVVSVLTVLHQDWLNGKEYKCKVSNKALPAPIEKTISKAKG |
| | | | QPREPQVCTLPPSREEMTKNQVSLSCAVKGFYPSDIAVEWESNGQ |
| | | | PENNYKTTPPVLDSDGSFFLVSKLTVDKSRWQQGNVFSCSVMHE |
| | | | ALHNHYTQKSLSLSPGK |
| Chain 2: | 1235 | Full Sequence | ITCPPPMSVEHADIWVKSYSLYSRERYICNSGFKRKAGTSSLTECV |
| IL15RA_Sushi- | | | LNKATNVAHWTTPSLKCIRDPALVHQRPAPPSGGSGGGGSGGGS |
| GS linker- | | | GGGGSGGNWVNVISDLKKIEDLIQSMHIDATLYTESDVHPSCKVT |
| IL15-GS | | | AMKCFLLELQVISLESGDASIHDTVENLIILANNSLSSNGNVTESGC |
| linker- | | | KECEELEEKNIKEFLQSFVHIVQMFINTSGGGGSGGGGSDKTHTCP |
| Fc_Knob_Cys_ | | | PCPAPELLGGPSVFLFPPKPKDTLMISRTPEVTCVVVDVSHEDPEV |
| N297A | | | KFNWYVDGVEVHNAKTKPREEQYASTYRVVSVLTVLHQDWLNG |
| | | | KEYKCKVSNKALPAPIEKTISKAKGQPREPQVYTLPPCREEMTKN |
| | | | QVSLWCLVKGFYPSDIAVEWESNGQPENNYKTTPPVLDSDGSFFL |
| | | | YSKLTVDKSRWQQGNVFSCSVMHEALHNHYTQKSLSLSPGK |
| | 1236 | IL15RA Sushi | ITCPPPMSVEHADIWVKSYSLYSRERYICNSGFKRKAGTSSLTECV |
| | | | LNKATNVAHWTTPSLKCIRDPALVHQRPAPP |
| | 1243 | linker | SGGSGGGGSGGGSGGGGSGG |
| | 1238 | human IL-15 | NWVNVISDLKKIEDLIQSMHIDATLYTESDVHPSCKVTAMKCFLL |
| | | | ELQVISLESGDASIHDTVENLIILANNSLSSNGNVTESGCKECEELE |
| | | | EKNIKEFLQSFVHIVQMFINTS |
| | 1237 | linker | GGGGSGGGGS |
| | 1206 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSL |
| | | | SPGK |
| Chain 3: | 1239 | Full Sequence | DIQMTQSPSFLSASVGDRVTITCKASQNVADRVVWHQQKPGKAP |
| TCRVβ light | | | KALIYSSSHRYKGVPSRFSGSGSGTEFTLTISSLQPEDFATYFCQQF |
| chain (CDRs | | | KSYPLTFGQGTKLEIKRTVAAPSVFIFPPSDEQLKSGTASVVCLLN |
| underlined) | | | NFYPREAKVQWKVDNALQSGNSQESVTEQDSKDSTYSLSSTLTLS |
| | | | KADYEKHKVYACEVTHQGLSSPVTKSFNRGEC |
| | 1173 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQE |
| | | | KSYPLTFGQGTKLEIK |
| | 1171 | LC CDR1 | KASQNVADRVV |
| | 1167 | LC CDR2 | SSSHRYK |
| | 1008 | LC CDR3 | QQFKSYPLT |
| | 1203 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

TCRVβ/IL15 bispecific

| | | | |
|---|---|---|---|
| Chain 1: | 1228 | Full Sequence | QVQLVQSGAEVKKPGSSVKVSCKASGHDFRLTYIHWVRQAPGQG |
| TCRVβ- | | | LEWMGRVSAGSGNVKYNEKFKGRVTITADTSTSTAYMELSSLRS |
| Fc_Hole_Cys_ | | | EDTAVYYCAVSYYSYDVLDYWGQGTTVTVSSASTKGPSVFPLAP |
| N297A | | | SSKSTSGGTAALGCLVKDYFPEPVTVSWNSGALTSGVHTFPAVLQ |
| heavy chain | | | SSGLYSLSSVVTVPSSSLGTQTYICNVNHKPSNTKVDKRVEPKSCD |
| (CDRs | | | KTHTCPPCPAPELLGGPSVFLFPPKPKDTLMISRTPEVTCVVVDVS |
| underlined) | | | HEDPEVKFNWYVDGVEVHNAKTKPREEQYASTYRVVSVLTVLH |
| | | | QDWLNGKEYKCKVSNKALPAPIEKTISKAKGQPREPQVCTLPPSR |
| | | | EEMTKNQVSLSCAVKGFYPSDIAVEWESNGQPENNYKTTPPVLDS |
| | | | DGSFFLVSKLTVDKSRWQQGNVFSCSVMHEALHNHYTQKSLSLS |
| | | | PGK |
| | 1168 | anti-TCRvβ VH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRS |
| | | | EDTAVYYCAVSYYSYDVLDYWGQGTTVTVSS |
| | 1164 | HC CDR1 | GHDFRLTYIE |
| | 1229 | HC CDR1 | LTYIH |
| | 1234 | HC CDR1 | FRLTYIH |
| | 1165 | HC CDR2 | RVSAGSGNVKYNEKFKG |
| | 1005 | HC CDR3 | SYYSYDVLDY |
| | 1207 | Human IgG1 | ASTKGPSVFPLAPSSKSTSGGTAALGCLVKDYFPEPVTVSWNSGA |
| | | hole cys N297A | LTSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMIS |
| | | | RTPEVTCVVVDVSHEDPEVKFNWYVDGVEVHNAKTKPREEQYA |
| | | | STYRVVSVLTVLHQDWLNGKEYKCKVSNKALPAPIEKTISKAKG |
| | | | QPREPQVCTLPPSREEMTKNQVSLSCAVKGFYPSDIAVEWESNGQ |
| | | | PENNYKTTPPVLDSDGSFFLVSKLTVDKSRWQQGNVFSCSVMHE |
| | | | ALHNHYTQKSLSLSPGK |
| Chain 2: | 1240 | Full Sequence | NWVNVISDLKKIEDLIQSMHIDATLYTESDVHPSCKVTAMKCFLL |
| IL15-GS | | | ELQVISLESGDASIHDTVENLIILANNSLSSNGNVTESGCKECEELE |
| linker- | | | TEKNIKEFLQSFVHIVQMFINTSGGGGSGGGGSDKTHTCPPCPAPEL |
| Fc_Knob_Cys_ | | | LGGPSVFLFPPKPKDTLMISRTPEVTCVVVDVSHEDPEVKFNWYV |
| N297A | | | DGVEVHNAKTKPREEQYASTYRVVSVLTVLHQDWLNGKEYKCK |
| | | | VSNKALPAPIEKTISKAKGQPREPQVYTLPPCREEMTKNQVSLWC |
| | | | LVKGFYPSDIAVEWESNGQPENNYKTTPPVLDSDGSFFLYSKLTV |
| | | | DKSRWQQGNVFSCSVMHEALHNHYTQKSLSLSPGK |
| | 1238 | human IL-15 | NWVNVISDLKKIEDLIQSMHIDATLYTESDVHPSCKVTAMKCFLL |
| | | | ELQVISLESGDASIHDTVENLIILANNSLSSNGNVTESGCKECEELE |
| | | | EKNIKEFLQSFVHIVQMFINTS |
| | 1237 | linker | GGGGSGGGGS |
| | 1206 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSL |
| | | | SPGK |
| Chain 3: | 1239 | Full Sequence | DIQMTQSPSFLSASVGDRVTITCKASQNVADRVVWHQQKPGKAP |
| TCRVβ light | | | KALIYSSSHRYKGVPSRFSGSGSGTEFTLTISSLQPEDFATYFCQQF |
| chain (CDRs | | | KSYPLTFGQGTKLEIKRTVAAPSVFIFPPSDEQLKSGTASVVCLLN |
| underlined) | | | NFYPREAKVQWKVDNALQSGNSQESVTEQDSKDSTYSLSSTLTLS |
| | | | KADYEKHKVYACEVTHQGLSSPVTKSFNRGEC |
| | 1173 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQF |
| | | | KSYPLTFGQGTKLEIK |
| | 1171 | LC CDR1 | KASQNVADRVV |
| | 1167 | LC CDR2 | SSSHRYK |
| | 1008 | LC CDR3 | QQFKSYPLT |
| | 1203 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

BKM0186

| | | | |
|---|---|---|---|
| Chain 1 | 1228 | Full Sequence | QVQLVQSGAEVKKPGSSVKVSCKASGHDFRLTYIHWVRQAPGQG |
| (heavy chain) | | | LEWMGRVSAGSGNVKYNEKFKGRVTITADTSTSTAYMELSSLRS |
| (CDRs | | | EDTAVYYCAVSYYSYDVLDYWGQGTTVTVSSASTKGPSVFPLAP |
| underlined) | | | SSKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVCTLPPSR |
| | | | EEMTKNQVSLSCAVKGFYPSDIAVEWESNGQPENNYKTTPPVLDS |
| | | | DGSFFLVSKLTVDKSRWQQGNVFSCSVMHEALHNHYTQKSLSLS |
| | | | PGK |
| | 1168 | anti-TCRvβ VH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRS |
| | | | EDTAVYYCAVSYYSYDVLDYWGQGTTVTVSS |
| | 1164 | HC CDR1 | GHDFRLTYIH |
| | 1229 | HC CDR1 | LTYIH |
| | 1234 | HC CDR1 | FRLTYIH |
| | 1165 | HC CDR2 | RVSAGSGNVKYNEKFKG |
| | 1005 | HC CDR3 | SYYSYDVLDY |
| | 1207 | Human IgG1 | ASTKGPSVFPLAPSSKSTSGGTAALGCLVKDYFPEPVTVSWNSGA |
| | | hole cys N297A | LTSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMIS |
| | | | RTPEVTCVVVDVSHEDPEVKFNWYVDGVEVHNAKTKPREEQYA |
| | | | STYRVVSVLTVLHQDWLNGKEYKCKVSNKALPAPIEKTISKAKG |
| | | | QPREPQVCTLPPSREEMTKNQVSLSCAVKGFYPSDIAVEWESNGQ |
| | | | PENNYKTTPPVLDSDGSFFLVSKLTVDKSRWQQGNVFSCSVMHE |
| | | | ALHNHYTQKSLSLSPGK |
| Chain 2 | 1241 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFAQSISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQV |
| | | | YTLPPCREEMTKNQVSLWCLVKGFYPSDIAVEWESNGQPENNYK |
| | | | TTPPVLDSDGSFFLYSKLTVDKSRWQQGNVFSCSVMHEALHNHY |
| | | | TQKSLSLSPGK |
| | 1242 | IL-2 C125A | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFAQSUSTLT |
| | 1237 | linker | GGGGSGGGGS |
| | 1206 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSL |
| | | | SPGK |
| Chain 3 | 1239 | Full Sequence | DIQMTQSPSFLSASVGDRVTITCKASQNVADRVVWHQQKPGKAP |
| (light chain) | | | KALIYSSSHRYKGVPSRFSGSGSGTEFTLTISSLQPEDFATYFCQQF |
| (CDRs | | | KSYPLTFGQGTKLEIKRTVAAPSVFIFPPSDEQLKSGTASVVCLLN |
| underlined) | | | NFYPREAKVQWKVDNALQSGNSQESVTEQDSKDSTYSLSSTLTLS |
| | | | KADYEKHKVYACEVTHQGLSSPVIKSFNRGEC |
| | 1173 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQF |
| | | | KSYPLTFGQGTKLEIK |
| | 1171 | LC CDR1 | KASQNVADRVV |
| | 1167 | LC CDR2 | SSSHRYK |
| | 1008 | LC CDR3 | QQFKSYPLT |
| | 1203 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

mBKM0186

| | | | |
|---|---|---|---|
| Chain 1 | 1244 | Full Sequence | QVQLQQSGTELMKPGASVKISCKASGYTFSNYWIEWIKQRPGHGL |
| (heavy chain) | | | EWVGEILPGAGPTNYNEKFKGKATFTADSSSNTAYMQLSSLTSED |
| (CDRs | | | SAVYYCARTDYDYDWFAYWGQGTLVTVSAAKTTAPSVYPLAPV |
| underlined) | | | SGDTTGSSVTLGCLVKGYFPEPVTLTWNSGSLSSGVHTFPAVLQS |
| | | | DLYTLSSSVTVTSSTWPSQSITCNVAHPASSTKVDKKIEPKSCDKT |
| | | | HTCPPCPAPNAAGGPSVFIFPPKIKDVLMISLSPIVTCVVVDVSEDD |
| | | | PDVQISWFVNNVEVHTAQTQTHREDYNSTLRVVSALPIQHQDWM |
| | | | SGKEFKCKVNNKDLGAPIERTISKPKGSVRAPQVCVLPPPEEEMTK |
| | | | KQVTLSCAVTDFMPEDIYVEWTNNGKTELNYKNTEPVLDSDGSY |
| | | | FMVSKLRVEKKNWVERNSYSCSVVHEGLHNHHTTKSFSRTPGKE |
| | | | PEA |
| | 1245 | Full Sequence | QVQLQQSGTELMKPGASVKISCKASGYTFSNYWIEWIKQRPGHGL |
| | | without tag | EWVGEILPGAGPTNYNEKFKGKATFTADSSSNTAYMQLSSLTSED |
| | | | SAVYYCARTDYDYDWFAYWGQGTLVTVSAAKTTAPSVYPLAPV |
| | | | SGDTTGSSVTLGCLVKGYFPEPVTLTWNSGSLSSGVHTFPAVLQS |
| | | | DLYTLSSSVTVTSSTWPSQSITCNVAHPASSTKVDKKIEPKSCDKT |
| | | | HTCPPCPAPNAAGGPSVFIFPPKIKDVLMISLSPIVTCVVVDVSEDD |
| | | | PDVQISWFVNNVEVHTAQTQTHREDYNSTLRVVSALPIQHQDWM |
| | | | SGKEFKCKVNNKDLGAPIERTISKPKGSVRAPQVCVLPPPEEEMTK |
| | | | KQVTLSCAVTDFMPEDIYVEWTNNGKTELNYKNTEPVLDSDGSY |
| | | | FMVSKLRVEKKNWVERNSYSCSVVHEGLHNHHTTKSFSRTPGK |
| | 1246 | anti-TCRvβ VH | QVQLQQSGTELMKPGASVKISCKASGYTFSNYWIEWIKQRPGHGL |
| | | from murine | EWVGEILPGAGPTNYNEKFKGKATFTADSSSNTAYMQLSSLTSED |
| | | MR5-2 | SAVYYCARTDYDYDWFAYWGQGTLVTVSAAKTTAPSVYPLAPV |
| | | | SGDTTGSSVTLGCLVKGYFPEPVTLTWNSGSLSSGVHTFPAVLQS |
| | | | DLYTLSSSVTVTSS |
| | 1247 | HC CDR1 | GYTFSNYWIE |
| | 1248 | HC CDR2 | EILPGAGPTNYNEKFKG |
| | 1249 | HC CDR3 | TDYDYDWFAY |
| | 1318 | HC CDR3 | FPAVLQSDLY |
| | 1264 | mouse Fc heavy | TWPSQSITCNVAHPASSTKVDKKIEPKSCDKTHTCPPCPAPNAAGG |
| | | chain | PSVFIFPPKIKDVLMISLSPIVTCVVVDVSEDDPDVQISWFVNNVEV |
| | | comprising | HTAQTQTHREDYNSTLRVVSALPIQHQDWMSGKEFKCKVNNKDL |
| | | LALAPG | GAPIERTISKPKGSVRAPQVCVLPPPEEEMTKKQVTLSCAVTDFMP |
| | | mutations | EDIYVEWTNNGKTELNYKNTEPVLDSDGSYFMVSKLRVEKKNW |
| | | (L234A L235A | VERNSYSCSVVHEGLHNHHTTKSFSRTPGK |
| | | and P329G) | |
| | 1265 | Tag | EPEA |
| Chain 2 | 1266 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFCQSIISTLTGGGGS |
| | | | GGGGSDKTHTCPPCPAPNAAGGPSVFIFPPKIKDVLMISLSPIVTCV |
| | | | VVDVSEDDPDVQISWFVNNVEVHTAQTQTHREDYNSTLRVVSAL |
| | | | PIQHQDWMSGKEFKCKVNNKDLGAPIERTISKPKGSVRAPQVYVL |
| | | | PPCEEEMTKKQVTLWCMVTDFMPEDIYVEWTNNGKTELNYKNT |
| | | | EPVLDSDGSYFMYSKLRVEKKNWVERNSYSCSVVHEGLHNHHTT |
| | | | KSFSRTPGKGGGGSHHHHHHHH |
| | 1267 | Full Sequence | APTSSSTKKTQLQLEHLLLDLQMILNGINNYKNPKLTRMLTFKFY |
| | | without tag | MPKKATELKHLQCLEEELKPLEEVLNLAQSKNFHLRPRDLISNINV |
| | | | IVLELKGSETTFMCEYADETATIVEFLNRWITFCQSIISTLTGGGGS |
| | | | GGGGSDKTHTCPPCPAPNAAGGPSVFIFPPKIKDVLMISLSPIVTCV |
| | | | VVDVSEDDPDVQISWFVNNVEVHTAQTQTHREDYNSTLRVVSAL |
| | | | PIQHQDWMSGKEFKCKVNNKDLGAPIERTISKPKGSVRAPQVYVL |
| | | | PPCEEEMTKKQVTLWCMVTDFMPEDIYVEWTNNGKTELNYKNT |
| | | | EPVLDSDGSYFMYSKLRVEKKNWVERNSYSCSVVHEGLHNHHTT |
| | | | KSFSRTPGK |
| | 1268 | IL-2 | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFCQSIISTLT |
| | 1237 | linker | GGGGSGGGGS |
| | 1269 | mouse Fc heavy | DKTHTCPPCPAPNAAGGPSVFIFPPKIKDVLMISLSPIVTCVVVDVS |
| | | chain | EDDPDVQISWFVNNVEVHTAQTQTHREDYNSTLRVVSALPIQHQD |
| | | comprising | WMSGKEFKCKVNNKDLGAPIERTISKPKGSVRAPQVYVLPPCEEE |
| | | LALAPG | MTKKQVTLWCMVTDFMPEDIYVEWTNNGKTELNYKNTEPVLDS |
| | | mutations | DGSYFMYSKLRVEKKNWVERNSYSCSVVHEGLHNHHTTKSFSRT |
| | | (L234A L235A | PGK |
| | | and P329G) | |
| | 1270 | Tag | GGGGSHHHHHHHH |
| Chain 3 | 1271 | Full Sequence | DIVMSQSPSSLAVSVGEKVTMSCKSSQSLLYSGNQKNYLAWYQQ |
| (light chain) | | | KPGQSPKLLIYWASTRESGVPDRFTGSGSGTDFTLTINSVKAEDLT |
| (CDRs | | | VYYCQQYYGYPRTFGGGTKVEIKRADAAPTVSIFPPSSEQLTSGGA |
| underlined) | | | SVVCFLNNFYPKDINVKWKIDGSERQNGVLNSWTDQDSKDSTYS |
| | | | MSSTLTLTKDEYERHNSYTCEATHKTSTSPIVKSFNRNEC |
| | 1252 | anti-TCRvβ VL | DIVMSQSPSSLAVSVGEKVTMSCKSSQSLLYSGNQKNYLAWYQQ |
| | | from murine | KPGQSPKLLIYWASTRESGVPDRFTGSGSGTDFTLTINSVKAEDLT |
| | | MR5-2 | VYYCQQYYGYPRTFGGGTKVEIK |
| | 1253 | LC CDR1 | KSSQSLLYSGN |
| | 1254 | LC CDR2 | PKLLIYWA |
| | 1255 | LC CDR3 | QQYYGYPRT |
| | 1256 | Immunoglobulin | RADAAPTVSIFPPSSEQLTSGGASVVCFLNNFYPKDINVKWKIDGS |
| | | constant region | ERQNGVLNSWTDQDSKDSTYSMSSTLTLTKDEYERHNSYTCEAT |
| | | | HKTSTSPIVKSFNRNEC |

mBKM0281

| | | | |
|---|---|---|---|
| Chain 1 | 1257 | Full Sequence | QVQLQQSGTELMKPGASVKISCKASGYTFSNYWIEWIKQRPGHGL |
| (heavy chain) | | | EWVGEILPGAGPTNYNEKFKGKATFTADSSSNTAYMQLSSLTSED |
| (CDRs | | | SAVYYCARTDYDYDWFAYWGQGTLVTVSAAKTTAPSVYPLAPV |
| underlined) | | | SGDTTGSSVTLGCLVKGYFPEPVTLTWNSGSLSSGVHTFPAVLQS |
| | | | DLYTLSSSVTVTSSTWPSQSITCNVAHPASSTKVDKKIEPKSCDKT |
| | | | HTCPPCPAPNAAGGPSVFIFPPKIKDVLMISLSPIVTCVVVDVSEDD |
| | | | PDVQISWFVNNVEVHTAQTQTHREDYNSTLRVVSALPIQHQDWM |
| | | | SGKEFKCKVNNKDLGAPIERTISKPKGSVRAPQVYVLPPPEEEMTK |
| | | | KQVTLTCMVTDFMPEDIYVEWINNGKTELNYKNTEPVLDSDGSY |
| | | | FMYSKLRVEKKNWVERNSYSCSVVHEGLHNHHTTKSFSRTPGKE |
| | | | PEA |
| | 1246 | anti-TCRvβ VH | QVQLQQSGTELMKPGASVKISCKASGYTFSNYWIEWIKQRPGHGL |
| | | from murine | EWVGEILPGAGPTNYNEKFKGKATFTADSSSNTAYMQLSSLTSED |
| | | MR5-2 | SAVYYCARTDYDYDWFAYWGQGTLVTVSAAKTTAPSVYPLAPV |
| | | | SGDTTGSSVTLGCLVKGYFPEPVTLTWNSGSLSSGVHTFPAVLQS |
| | | | DLYTLSSSVTVTSS |
| | 1247 | HC CDR1 | GYTFSNYWIE |
| | 1248 | HC CDR2 | EILPGAGPTNYNEKFKG |
| | 1249 | HC CDR3 | TDYDYDWFAY |
| | 1250 | mouse Fc heavy | TWPSQSITCNVAHPASSTKVDKKIEPKSCDKTHTCPPCPAPNAAGG |
| | | chain | PSVFIFPPKIKDVLMISLSPIVTCVVVDVSEDDPDVQISWFVNNVEV |
| | | comprising | HTAQTQTHREDYNSTLRVVSALPIQHQDWMSGKEFKCKVNNKDL |
| | | LALAPG | GAPIERTISKPKGSVRAPQVYVLPPPEEEMTKKQVTLTCMVTDFM |
| | | mutations | PEDIYVEWTNNGKTELNYKNTEPVLDSDGSYFMYSKLRVEKKNW |
| | | (L234A L235A | VERNSYSCSVVHEGLHNHHTTKSFSRTPGKEPEA |
| | | and P329G) | |
| Chain 2 | 1251 | Full Sequence | DIVMSQSPSSLAVSVGEKVTMSCKSSQSLLYSGNQKNYLAWYQQ |
| (light chain- | | | KPGQSPKLLIYWASTRESGVPDRFTGSGSGTDFTLTINSVKAEDLT |
| IL2) | | | VYYCQQYYGYPRTFGGGTKVEIKRADAAPTVSIFPPSSEQLTSGGA |
| (CDRs | | | SVVCFLNNFYPKDINVKWKIDGSERQNGVLNSWTDQDSKDSTYS |
| underlined) | | | MSSTLTLTKDEYERHNSYTCEATHKTSTSPIVKSFNRNECGGGGSG |
| | | | GGGSGGGGSAPTSSSTKKTQLQLEHLLLDLQMILNGINNYKNPKL |
| | | | TRMLTFKFYMPKKATELKHLQCLEEELKPLEEVLNLAQSKNFHLR |
| | | | PRDLISNINVIVLELKGSETTFMCEYADETATIVEFLNRWITFCQSII |
| | | | STLT |
| | 1252 | anti-TCRvβ VL | DIVMSQSPSSLAVSVGEKVTMSCKSSQSLLYSGNQKNYLAWYQQ |
| | | from murine | KPGQSPKLLIYWASTRESGVPDRFTGSGSGTDFTLTINSVKAEDLT |
| | | MR5-2 | VYYCQQYYGYPRTFGGGTKVEIK |
| | 1253 | LC CDR1 | KSSQSLLYSGN |
| | 1254 | LC CDR2 | PKLLIYWA |
| | 1255 | LC CDR3 | QQYYGYPRT |
| | 1256 | Immunoglobulin | RADAAPTVSIFPPSSEQLTSGGASVVCFLNNFYPKDINVKWKIDGS |
| | | constant region | ERQNGVLNSWTDQDSKDSTYSMSSTLTLTKDEYERHNSYTCEAT |
| | | | HKTSTSPIVKSFNRNEC |
| | 1258 | linker | GGGGSGGGGSGGGGS |
| | 268 | IL-2 | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFCQSIISTLT |

BMM0317 (LL-7)

| | | | |
|---|---|---|---|
| Chain 1: | 1228 | Full Sequence | QVQLVQSGAEVKKPGSSVKVSCKASGHDFRLTYIHWVRQAPGQG |
| TCRVβ- | | | LEWMGRVSAGSGNVKYNEKFKGRVTITADTSTSTAYMELSSLRS |
| Fc Hole_Cys_ | | | EDTAVYYCAVSYYSYDVLDYWGQGTTVTVSSASTKGPSVFPLAP |
| N297A heavy | | | SSKSTSGGTAALGCLVKDYFPEPVTVSWNSGALTSGVHTFPAVLQ |
| chain (CDRs | | | SSGLYSLSSVVTVPSSSLGTQTYICNVNHKPSNTKVDKRVEPKSCD |
| underlined) | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVCTLPPSR |
| | | | EEMTKNQVSLSCAVKGFYPSDIAVEWESNGQPENNYKTTPPVLDS |
| | | | DGSFFLVSKLTVDKSRWQQGNVFSCSVMHEALHNHYTQKSLSLS |
| | | | PGK |
| | 1168 | anti-TCRvβ VH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRS |
| | | | EDTAVYYCAVSYYSYDVLDYWGQGTTVTVSS |
| | 1164 | HC CDR1 | GHDFRLTYIH |
| | 1229 | HC CDR1 | LTYIH |
| | 1234 | HC CDR1 | FRLTYIH |
| | 1165 | HC CDR2 | RVSAGSGNVKYNEKFKG |
| | 1005 | HC CDR3 | SYYSYDVLDY |
| | 1207 | Human IgG1 | ASTKGPSVFPLAPSSKSTSGGTAALGCLVKDYFPEPVTVSWNSGA |
| | | hole cys N297A | LTSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMIS |
| | | | RTPEVTCVVVDVSHEDPEVKFNWYVDGVEVHNAKTKPREEQYA |
| | | | STYRVVSVLTVLHQDWLNGKEYKCKVSNKALPAPIEKTISKAKG |
| | | | QPREPQVCTLPPSREEMTKNQVSLSCAVKGFYPSDIAVEWESNGQ |
| | | | PENNYKTTPPVLDSDGSFFLVSKLTVDKSRWQQGNVFSCSVMHE |
| | | | ALHNHYTQKSLSLSPGK |
| Chain 2: | 1272 | Full Sequence | DCDIEGKDGKQYESVLMVSIDQLLDSMKEIGSNCLNNEFNFFKRHI |
| IL-7-GS | | | CDANKEGMFLFRAARKLRQFLKMNSTGDFDLHLLKVSEGTTILLN |
| linker-IL15- | | | CTGQVKGRKPAALGEAQPTKSLEENKSLKEQKKLNDLCFLKRLL |
| GS linker- | | | QEIKTCWNKILMGTKEHGGGGSGGGGSDKTHTCPPCPAPELLGGP |
| Fc_Knob_Cys_ | | | SVFLFPPKPKDTLMISRTPEVTCVVVDVSHEDPEVKFNWYVDGVE |
| N297A | | | VHNAKTKPREEQYASTYRVVSVLTVLHQDWLNGKEYKCKVSNK |
| | | | ALPAPIEKTISKAKGQPREPQVYTLPPCREEMTKNQVSLWCLVKG |
| | | | FYPSDIAVEWESNGQPENNYKTTPPVLDSDGSFFLYSKLTVDKSR |
| | | | WQQGNVFSCSVMHEALHNHYTQKSLSLSPGK |
| | 1273 | IL-7 | DCDIEGKDGKQYESVLMVSIDQLLDSMKEIGSNCLNNEFNFFKRHI |
| | | | CDANKEGMFLFRAARKLRQFLKMNSTGDFDLHLLKVSEGTTILLN |
| | | | CTGQVKGRKPAALGEAQPTKSLEENKSLKEQKKLNDLCFLKRLL |
| | | | QEIKTCWNKILMGTKEH |
| | 1237 | linker | GGGGSGGGGS |
| | 1206 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSL |
| | | | SPGK |
| Chain 3: | 1239 | Full Sequence | DIQMTQSPSFLSASVGDRVTITCKASQNVADRVVWHQQKPGKAP |
| TCRVβ light | | | KALIYSSSHRYKGVPSRFSGSGSGTEFTLTISSLQPEDFATYFCQQF |
| chain (CDRs | | | KSYPLTFGQGTKLEIKRTVAAPSVFIFPPSDEQLKSGTASVVCLLN |
| underlined) | | | NFYPREAKVQWKVDNALQSGNSQESVTEQDSKDSTYSLSSTLTLS |
| | | | KADYEKHKVYACEVTHQGLSSPVTKSFNRGEC |
| | 1173 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQF |
| | | | KSYPLTFGQGTKLEIK |
| | 1171 | LC CDR1 | KASQNVADRVV |
| | 1167 | LC CDR2 | SSSHRYK |
| | 1008 | LC CDR3 | QQFKSYPLT |
| | 1203 | Immunoglobulin C | RTVAAPSVFIFPPSDEQLKSGTASVVCLLNNFYPREAKVQWKVDN |
| | | kappa constant | ALQSGNSQESVTEQDSKDSTYSLSSTLTLSKADYEKHKVYACEVT |
| | | region | HQGLSSPVTKSFNRGEC |

BLM0319 (IL-12)

| | | | |
|---|---|---|---|
| Chain 1: | 1228 | Full Sequence | QVQLVQSGAEVKKPGSSVKVSCKASGHDFRLTYIHWVRQAPGQG |
| TCRVβ- | | | LEWMGRVSAGSGNVKYNEKFKGRVTITADTSTSTAYMELSSLRS |
| Fc_Hole_Cys_ | | | EDTAVYYCAVSYYSYDVLDYWGQGTTVTVSSASTKGPSVFPLAP |
| N297A heavy | | | SSKSTSGGTAALGCLVKDYFPEPVTVSWNSGALTSGVHTFPAVLQ |
| chain (CDRs | | | SSGLYSLSSVVTVPSSSLGTQTYICNVNHKPSNTKVDKRVEPKSCD |
| underlined) | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVCTLPPSR |
| | | | EEMTKNQVSLSCAVKGFYPSDIAVEWESNGQPENNYKTTPPVLDS |
| | | | DGSFFLVSKLTVDKSRWQQGNVFSCSVMHEALHNHYTQKSLSLS |
| | | | PGK |
| | 168 | anti-TCRvβ VH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRS |
| | | | EDTAVYYCAVSYYSYDVLDYWGQGTTVTVSS |
| | 1164 | HC CDR1 | GHDFRLTYIH |
| | 1229 | HC CDR1 | LTYIH |
| | 1234 | HC CDR1 | FRLTYIH |
| | 1165 | HC CDR2 | RVSAGSGNVKYNEKFKG |
| | 1005 | HC CDR3 | SYYSYDVLDY |
| | 1207 | Human IgG1 | ASTKGPSVFPLAPSSKSTSGGTAALGCLVKDYFPEPVTVSWNSGA |
| | | hole cys N297A | LTSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMIS |
| | | | RTPEVTCVVVDVSHEDPEVKFNWYVDGVEVHNAKTKPREEQYA |
| | | | STYRVVSVLTVLHQDWLNGKEYKCKVSNKALPAPIEKTISKAKG |
| | | | QPREPQVCTLPPSREEMTKNQVSLSCAVKGFYPSDIAVEWESNGQ |
| | | | PENNYKTTPPVLDSDGSFFLVSKLTVDKSRWQQGNVFSCSVMHE |
| | | | ALHNHYTQKSLSLSPGK |
| Chain 2: | 1275 | Full Sequence | IWELKKDVYVVELDWYPDAPGEMVVLTCDTPEEDGITWTLDQSS |
| IL-12-GS | | | EVLGSGKTLTIQVKEFGDAGQYTCHKGGEVLSHSLLLLHKKEDGI |
| linker-IL15- | | | WSTDILKDQKEPKNKTFLRCEAKNYSGRFTCWWLTTISTDLTFSV |
| GS linker- | | | KSSRGSSDPQGVTCGAATLSAERVRGDNKEYEYSVECQEDSACPA |
| Fc_Knob_Cys_ | | | AEESLPIEVMVDAVHKLKYENYTSSFFIRDIIKPDPPKNLQLKPLKN |
| N297A | | | SRQVEVSWEYPDTWSTPHSYFSLTFCVQVQGKSKREKKDRVFTD |
| | | | KTSATVICRKNASISVRAQDRYYSSSWSEWASVPCSGGGGSGGGG |
| | | | SGGGGSRNLPVATPDPGMFPCLHHSQNLLRAVSNMLQKARQTLE |
| | | | FYPCTSEEIDHEDITKDKTSTVEACLPLELTKNESCLNSRETSFITNG |
| | | | SCLASRKTSFMMALCLSSIYEDLKMYQVEFKTMNAKLLMDPKRQ |
| | | | IFLDQNMLAVIDELMQALNFNSETVPQKSSLEEPDFYKTKIKLCILL |
| | | | HAFRIRAVTIDRVMSYLNASGGGGSGGGGSDKTHTCPPCPAPELL |
| | | | GGPSVFLFPPKPKDTLMISRTPEVTCVVVDVSHEDPEVKFNWYVD |
| | | | GVEVHNAKTKPREEQYASTYRVVSVLTVLHQDWLNGKEYKCKV |
| | | | SNKALPAPIEKTISKAKGQPREPQVYTLPPCREEMTKNQVSLWCL |
| | | | VKGFYPSDIAVEWESNGQPENNYKTTPPVLDSDGSFFLYSKLTVD |
| | | | KSRWQQGNVFSCSVMHEALHNHYTQKSLSLSPGK |
| | 1276 | IL-12 | IWELKKDVYVVELDWYPDAPGEMVVLTCDTPEEDGITWTLDQSS |
| | | | EVLGSGKTLTIQVKEFGDAGQYTCHKGGEVLSHSLLLLHKKEDGI |
| | | | WSTDILKDQKEPKNKTFLRCEAKNYSGRFTCWWLTTISTDLTFSV |
| | | | KSSRGSSDPQGVTCGAATLSAERVRGDNKEYEYSVECQEDSACPA |
| | | | AEESLPIEVMVDAVHKLKYENYTSSFFIRDIIKPDPPKNLQLKPLKN |
| | | | SRQVEVSWEYPDTWSTPHSYFSLTFCVQVQGKSKREKKDRVFTD |
| | | | KTSATVICRKNASISVRAQDRYYSSSWSEWASVPCSGGGGSGGGG |
| | | | SGGGGSRNLPVATPDPGMFPCLHHSQNLLRAVSNMLQKARQTLE |
| | | | FYPCTSEEIDHEDITKDKTSTVEACLPLELTKNESCLNSRETSFITNG |
| | | | SCLASRKTSFMMALCLSSIYEDLKMYQVEFKTMNAKLLMDPKRQ |
| | | | IFLDQNMLAVIDELMQALNFNSETVPQKSSLEEPDFYKTKIKLCILL |
| | | | HAFRIRAVTIDRVMSYLNAS |
| | 1277 | IL-12 beta | IWELKKDVYVVELDWYPDAPGEMVVLTCDTPEEDGITWTLDQSS |
| | | subunit | EVLGSGKTLTIQVKEFGDAGQYTCHKGGEVLSHSLLLLHKKEDGI |
| | | | WSTDILKDQKEPKNKTFLRCEAKNYSGRFTCWWLTTISTDLTFSV |
| | | | KSSRGSSDPQGVTCGAATLSAERVRGDNKEYEYSVECQEDSACPA |
| | | | AEESLPIEVMVDAVHKLKYENYTSSFFIRDIIKPDPPKNLQLKPLKN |
| | | | SRQVEVSWEYPDTWSTPHSYFSLTFCVQVQGKSKREKKDRVFTD |
| | | | KTSATVICRKNASISVRAQDRYYSSSWSEWASVPC |
| | 1278 | Linker | SGGGGSGGGGSGGGGS |
| | 1279 | IL-12 alpha | RNLPVATPDPGMFPCLHHSQNLLRAVSNMLQKARQTLEFYPCTSE |
| | | subunit | EIDHEDITKDKTSTVEACLPLELTKNESCLNSRETSFITNGSCLASR |
| | | | KTSFMMALCLSSIYEDLKMYQVEFKTMNAKLLMDPKRQIFLDQN |
| | | | MLAVIDELMQALNFNSETVPQKSSLEEPDFYKTKIKLCILLHAFRIR |
| | | | AVTIDRVMSYLNAS |
| | 1237 | linker | GGGGSGGGGS |
| | 1206 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSL |
| | | | SPGK |
| Chain 3: | 1239 | Full Sequence | DIQMTQSPSFLSASVGDRVTITCKASQNVADRVVWHQQKPGKAP |
| TCRVβ light | | | KALIYSSSHRYKGVPSRFSGSGSGTEFTLTISSLQPEDFATYFCQQF |
| chain (CDRs | | | KSYPLTFGQGTKLEIKRTVAAPSVFIFPPSDEQLKSGTASVVCLLN |
| underlined) | | | NFYPREAKVQWKVDNALQSGNSQESVTEQDSKDSTYSLSSTLTLS |
| | | | KADYEKHKVYACEVTHQGLSSPVTKSFNRGEC |
| | 1173 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQF |
| | | | KSYPLTFGQGTKLEIK |
| | 1171 | LC CDR1 | KASQNVADRVV |
| | 1167 | LC CDR2 | SSSHRYK |
| | 1008 | LC CDR3 | QQFKSYPLT |
| | 1203 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

BLM0321 (IL-21)

| | | | |
|---|---|---|---|
| Chain 1: | 1228 | Full Sequence | QVQLVQSGAEVKKPGSSVKVSCKASGHDFRLTYIHWVRQAPGQG |
| TCRVβ- | | | LEWMGRVSAGSGNVKYNEKFKGRVTITADTSTSTAYMELSSLRS |
| Fc_Hole_Cys_ | | | EDTAVYYCAVSYYSYDVLDYWGQGTTVTVSSASTKGPSVFPLAP |
| N297A heavy | | | SSKSTSGGTAALGCLVKDYFPEPVTVSWNSGALTSGVHTFPAVLQ |
| chain (CDRs | | | SSGLYSLSSVVTVPSSSLGTQTYICNVNHKPSNTKVDKRVEPKSCD |
| underlined) | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVCTLPPSR |
| | | | EEMTKNQVSLSCAVKGFYPSDIAVEWESNGQPENNYKTTPPVLDS |
| | | | DGSFFLVSKLTVDKSRWQQGNVFSCSVMHEALHNHYTQKSLSLS |
| | | | PGK |
| | 1168 | anti-TCRvβ VH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRS |
| | | | EDTAVYYCAVSYYSYDVLDYWGQGTTVTVSS |
| | 1164 | HC CDR1 | GHDFRLTYIH |
| | 1229 | HC CDR1 | LTYIH |
| | 1234 | HC CDR1 | FRLTYIH |
| | 1165 | HC CDR2 | RVSAGSGNVKYNEKFKG |
| | 1005 | HC CDR3 | SYYSYDVLDY |
| | 1207 | Human IgG1 | ASTKGPSVFPLAPSSKSTSGGTAALGCLVKDYFPEPVTVSWNSGA |
| | | hole cys N297A | LTSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMIS |
| | | | RTPEVTCVVVDVSHEDPEVKFNWYVDGVEVHNAKTKPREEQYA |
| | | | STYRVVSVLTVLHQDWLNGKEYKCKVSNKALPAPIEKTISKAKG |
| | | | QPREPQVCTLPPSREEMTKNQVSLSCAVKGFYPSDIAVEWESNGQ |
| | | | PENNYKTTPPVLDSDGSFFLVSKLTVDKSRWQQGNVFSCSVMHE |
| | | | ALHNHYTQKSLSLSPGK |
| Chain 2: | 1288 | Full Sequence | QGQDRHMIRMRQLIDIVDQLKNYVNDLVPEFLPAPEDVETNCEW |
| IL-21-GS | | | SAFSCFQKAQLKSANTGNNERIINVSIKKLKRKPPSTNAGRRQKHR |
| linker-IL15- | | | LTCPSCDSYEKKPPKEFLERFKSLLQKMIHQHLSSRTHGSEDSGGG |
| GS linker- | | | GSGGGGSDKTHTCPPCPAPELLGGPSVFLFPPKPKDTLMISRTPEV |
| Fc Knob_Cys_ | | | TCVVVDVSHEDPEVKFNWYVDGVEVHNAKTKPREEQYASTYRV |
| N297A | | | VSVLTVLHQDWLNGKEYKCKVSNKALPAPIEKTISKAKGQPREPQ |
| | | | VYTLPPCREEMTKNQVSLWCLVKGFYPSDIAVEWESNGQPENNY |
| | | | KTTPPVLDSDGSFFLYSKLTVDKSRWQQGNVFSCSVMHEALHNH |
| | | | YTQKSLSLSPGK |
| | 1274 | IL-21 | QGQDRHMIRMRQLIDIVDQLKNYVNDLVPEFLPAPEDVETNCEW |
| | | | SAFSCFQKAQLKSANTGNNERIINVSIKKLKRKPPSTNAGRRQKHR |
| | | | LTCPSCDSYEKKPPKEFLERFKSLLQKMIHQHLSSRTHGSEDS |
| | 1237 | linker | GGGGSGGGGS |
| | 1206 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSL |
| | | | SPGK |
| Chain 3: | 1239 | Full Sequence | DIQMTQSPSFLSASVGDRVTITCKASQNVADRVVWHQQKPGKAP |
| TCRVβ light | | | KALIYSSSHRYKGVPSRFSGSGSGTEFTLTISSLQPEDFATYFCQQF |
| chain (CDRs | | | KSYPLTFGQGTKLEIKRTVAAPSVFIFPPSDEQLKSGTASVVCLLN |
| underlined) | | | NFYPREAKVQWKVDNALQSGNSQESVTEQDSKDSTYSLSSTLTLS |
| | | | KADYEKHKVYACEVTHQGLSSPVTKSFNRGEC |
| | 1173 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQF |
| | | | KSYPLTFGQGTKLEIK |
| | 1171 | LC CDR1 | KASQNVADRVV |
| | 1167 | LC CDR2 | SSSHRYK |
| | 1008 | LC CDR3 | QQFKSYPLT |
| | 1203 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

TABLE 33
Exemplary multispecific molecule that comprise a first domain that binds to a first
target molecule, wherein the exemplary target molecule comprises TCRVβ 6 (e.g., TCRVβ 6-5,
TCRVβ 6-2). Exemplary TCRβV comprises TCRVβ 6 (e.g., TCRVβ 6-5, TCRVβ 6-2 ).
Exemplary anti-TCRVβ 6 antibodies include, but are not limited to, BMM0306, BMM0354,
BMM0356, BMM0307, BMM0359, BMM0355, BMM0308, BMM0360, BMM0357, and
BKM0186 which bind to human TCRVβ 6 (e.g., TCRVβ 6-5, TCRVβ 6-2). CDRs are
underlined.
BMM0306

| | | |
|---|---|---|
| SEQ ID NO: 1289 | Heavy chain (VH- | QVQLVQSGAEVKKPGSSVKVSCKASGHDFRLTYIHWVRQAP |
| | CH)-Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 1239 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 1292 | IL-15Rαsushi-IL-15 | ITCPPPMSVEHADIWVKSYSLYSRERYICNSGFKRKAGTSSLT |
| | Heavy Chain Constant | ECVLNKATNVAHWTTPSLKCIRDPALVHQRPAPPSGGSGGG |
| | Domain (Fc Region)- | GSGGGSGGGGSGGGNWVNVISDLKKIEDLIQSMHIDATLYTE |
| | Knob | SDVHPSCKVTAMKCFLLELQVISLESGDASIHDTVENLIILAN |
| | | NSLSSNGNVTESGCKECEELEEKNIKEFLQSFVHIVQMFINTSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPREEQ |
| | | YASTYRVVSVLTVLHQDWLNGKEYKCKVSNKALPAPIEKTIS |
| | | KAKGQPREPQVYTLPPCREEMTKNQVSLWCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1167 | LC CDR2 | SSSHRYK |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1173 | VL | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMIKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1238 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVQMFINTS |
| SEQ ID NO: 1297 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVQMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0354

| | | |
|---|---|---|
| SEQ ID NO: 1289 | Heavy chain (VH- | QVQLVQSGAEVKKPGSSVKVSCKASGHDFRLTYIHWVRQAP |
| | CH-Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 1298 | Light chain (VL-CL) | DIQMTQSPSFLSASVGDRVTITCKASQNVADRVVWHQQKPG |
| | | KAPKALIYAASYLQSGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 1292 | IL-15Rαsushi-IL-15 | ITCPPPMSVEHADIWVKSYSLYSRERYICNSGFKRKAGTSSLT |
| | Heavy Chain Constant | ECVLNKATNVAHWTTPSLKCIRDPALVHQRPAPPSGGSGGG |
| | Domain (Fe Region)- | GSGGGSGGGGSGGGNWVNVISDLKKIEDLIQSMHIDATLYTE |
| | Knob | SDVHPSCKVTAMKCFLLELQVISLESGDASIHDTVENLIILAN |
| | | NSLSSNGNVTESGCKECEELEEKNIKEFLQSFVHIVQMFINTSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPREEQ |
| | | YASTYRVVSVLTVLHQDWLNGKEYKCKVSNKALPAPIEKTIS |
| | | KAKGQPREPQVYTLPPCREEMTKNQVSLWCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1299 | LC CDR2 | AASYLQS |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1300 | VL | DIQMTQSPSFLSASVGDRVTITCKASQNVADRVVWHQQKPGK |
| | | APKALIYAASYLQSGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1238 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVQMFINTS |
| SEQ ID NO: 1297 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVQMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0356

| | | |
|---|---|---|
| SEQ ID NO: 1289 | Heavy chain (VH- | QVQLVQSGAEVKKPGSSVKVSCKASGHDFRLTYIHWVRQAP |
| | CH)-Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 1301 | Light chain (VL-CL) | DIQMTQSPSFLSASVGDRVTITCKASQNVADRVVWHQQKPG |
| | | KAPKALIFAASYLQSGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 1292 | IL-15Rαsushi-IL-15 | ITCPPPMSVEHADIWVKSYSLYSRERYICNSGFKRKAGTSSLT |
| | Heavy Chain Constant | ECVLNKATNVAHWTTPSLKCIRDPALVHQRPAPPSGGSGGG |
| | Domain (Fc Region)- | GSGGGSGGGGSGGGNWVNVISDLKKIEDLIQSMHIDATLYTE |
| | Knob | SDVHPSCKVTAMKCFLLELQVISLESGDASIHDTVENLIILAN |
| | | NSLSSNGNVTESGCKECEELEEKNIKEFLQSFVHIVQMFINTSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPREEQ |
| | | YASTYRVVSVLTVLHQDWLNGKEYKCKVSNKALPAPIEKTIS |
| | | KAKGQPREPQVYTLPPCREEMTKNQVSLWCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1299 | LC CDR2 | AASYLQS |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1303 | VL | DIQMTQSPSFLSASVGDRVTITCKASQNVADRVVWHQQKPGK |
| | | APKALIFAASYLQSGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1238 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVQMFINTS |
| SEQ ID NO: 1297 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVQMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0307

| | | |
|---|---|---|
| SEQ ID NO: 1289 | Heavy chain (VH- | QVQLVQSGAEVKKPGSSVKVSCKASGHDFRLTYIHWVRQAPG |
| | CH)-Hole | QGLEWMGRVSAGSGNVKYNEKFKGRVTITADTSTSTAYMELS |
| | | SLRSEDTAVYYCAVSYYSYDVLDYWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | ASTYRVVSVLTVLHQDWLNGKEYKCKVSNKALPAPIEKTISK |
| | | AKGQPREPQVCTLPPSREEMTKNQVSLSCAVKGFYPSDIAVEW |
| | | ESNGQPENNYKTTPPVLDSDGSFFLVSKLTVDKSRWQQGNVF |
| | | SCSVMHEALHNHYTQKSLSLSP |
| SEQ ID NO: 1239 | Light chain (VL-CL) | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 1304 | IL-15Rαsushi-IL-15 | ITCPPPMSVEHADIWVKSYSLYSRERYICNSGFKRKAGTSSLTE |
| | Heavy Chain Constant | CVLNKATNVAHWTTPSLKCIRDPALVHQRPAPPSGGSGGGGS |
| | Domain (Fc Region)- | GGGSGGGGSGGGNWVNVISDLKKIEDLIQSMHIDATLYTESDV |
| | Knob | HPSCKVTAMKCFLLELQVISLESGDASIHDTVENLIILANNSLSS |
| | | NGNVTESGCKECEELEEKNIKEFLQSFVHIVAMFINTSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYASTYR |
| | | VVSVLTVLHQDWLNGKEYKCKVSNKALPAPIEKTISKAKGQP |
| | | REPQVYTLPPCREEMTKNQVSLWCLVKGFYPSDIAVEWESNG |
| | | QPENNYKTTPPVLDSDGSFFLYSKLTVDKSRWQQGNVFSCSV |
| | | MHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1167 | LC CDR2 | SSSHRYK |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1173 | VL | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1307 | AA 49-162 of IL-15 | NWVNVISDLKKIEDLIQSMHIDATLYTESDVHPSCKVTAMKCF |
| | | LLELQVISLESGDASIHDTVENLILANNSLSSNGNVTESGCKEC |
| | | EELEEKNIKEFLQSFVHIVAMFINTS |
| SEQ ID NO: 1308 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVAMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0359

| | | |
|---|---|---|
| SEQ ID NO: 1289 | Heavy chain (VH- | QVQLVQSGAEVKKPGSSVKVSCKASGHDFRLTYIHWVRQAPG |
| | (CH)-Hole | QGLEWMGRVSAGSGNVKYNEKFKGRVTITADTSTSTAYMELS |
| | | SLRSEDTAVYYCAVSYYSYDVLDYWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | ASTYRVVSVLTVLHQDWLNGKEYKCKVSNKALPAPIEKTISK |
| | | AKGQPREPQVCTLPPSREEMTKNQVSLSCAVKGFYPSDIAVEW |
| | | ESNGQPENNYKTTPPVLDSDGSFFLVSKLTVDKSRWQQGNVF |
| | | SCSVMHEALHNHYTQKSLSLSP |
| SEQ ID NO: 1298 | Light chain (VL-CL) | DIQMTQSPSFLSASVGDRVTITCKASQNVADRVVWHQQKPGK |
| | | APKALIYAASYLQSGVPSRFSGSGSGTEFTLTISSLQPEDFATYF |
| | | CQQFKSYPLTFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 1304 | IL-15Rαsushi-IL-15 | ITCPPPMSVEHADIWVKSYSLYSRERYICNSGFKRKAGTSSLTE |
| | Heavy Chain Constant | CVLNKATNVAHWTTPSLKCIRDPALVHQRPAPPSGGSGGGGS |
| | Domain (Fc Region)- | GGGSGGGGSGGGNWVNVISDLKKIEDLIQSMHIDATLYTESDV |
| | Knob | HPSCKVTAMKCFLLELQVISLESGDASIHDTVENLIILANNSLSS |
| | | NGNVTESGCKECEELEEKNIKEFLQSFVHIVAMFINTSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYASTYR |
| | | VVSVLTVLHQDWLNGKEYKCKVSNKALPAPIEKTISKAKGQP |
| | | REPQVYTLPPCREEMTKNQVSLWCLVKGFYPSDIAVEWESNG |
| | | QPENNYKTTPPVLDSDGSFFLYSKLTVDKSRWQQGNVFSCSV |
| | | MHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1299 | LC CDR2 | AASYLQS |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1300 | VL | DIQMTQSPSFLSASVGDRVTITCKASQNVADRVVWHQQKPGK |
| | | APKALIYAASYLQSGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1307 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVAMFINTS |
| SEQ ID NO: 1308 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVAMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0355

| | | |
|---|---|---|
| SEQ ID NO: 1289 | Heavy chain (VH- | QVQLVQSGAEVKKPGSSVKVSCKASGHDFRLTYIHWVRQAP |
| | CH)-Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 1301 | Light chain (VL-CL) | DIQMTQSPSFLSASVGDRVTITCKASQNVADRVVWHQQKPGK |
| | | APKALIFAASYLQSGVPSRFSGSGSGTEFTLTISSLQPEDFATYF |
| | | CQQFKSYPLTFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 1304 | IL-15Rαsushi-IL-15 | ITCPPPMSVEHADIWVKSYSLYSRERYICNSGFKRKAGTSSLTE |
| | Heavy Chain Constant | CVLNKATNVAHWTTPSLKCIRDPALVHQRPAPPSGGSGGGGS |
| | Domain (Fc Region)- | GGGSGGGGSGGGNWVNVISDLKKIEDLIQSMHIDATLYTESDV |
| | Knob | HPSCKVTAMKCFLLELQVISLESGDASIHDTVENLIILANNSLSS |
| | | NGNVTESGCKECEELEEKNIKEFLQSFVHIVAMFINTSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYASTYR |
| | | VVSVLTVLHQDWLNGKEYKCKVSNKALPAPIEKTISKAKGQP |
| | | REPQVYTLPPCREEMTKNQVSLWCLVKGFYPSDIAVEWESNG |
| | | QPENNYKTTPPVLDSDGSFFLYSKLTVDKSRWQQGNVFSCSV |
| | | MHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1299 | LC CDR2 | AASYLQS |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1303 | VL | DIQMTQSPSFLSASVGDRVTITCKASQNVADRVVWHQQKPGK |
| | | APKALIFAASYLQSGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1307 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVAMFINTS |
| SEQ ID NO: 1308 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVAMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0308

| | | |
|---|---|---|
| SEQ ID NO: 1289 | Heavy chain (VH- | QVQLVQSGAEVKKPGSSVKVSCKASGHDFRLTYIHWVRQAPG |
| | CH)-Hole | QGLEWMGRVSAGSGNVKYNEKFKGRVTITADTSTSTAYMELS |
| | | SLRSEDTAVYYCAVSYYSYDVLDYWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | ASTYRVVSVLTVLHQDWLNGKEYKCKVSNKALPAPIEKTISK |
| | | AKGQPREPQVCTLPPSREEMTKNQVSLSCAVKGFYPSDIAVEW |
| | | ESNGQPENNYKTTPPVLDSDGSFFLVSKLTVDKSRWQQGNVF |
| | | SCSVMHEALHNHYTQKSLSLSP |
| SEQ ID NO: 1239 | Light chain (VL-CL) | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 1309 | IL-15Rαsushi-IL-15 | ITCPPPMSVEHADIWVKSYSLYSRERYICNSGFKRKAGTSSLTE |
| | Heavy Chain Constant | CVLNKATNVAHWTTPSLKCIRDPALVHQRPAPPSGGSGGGGS |
| | Domain (Fc Region)- | GGGSGGGGSGGGNWVNVISDLKKIEDLIQSMHIDATLYTESDV |
| | Knob | HPSCKVTAMKCFLLELQVISLESGDASIHDTVEQLIILANNSLSS |
| | | NGNVTESGCKECEELEEKNIKEFLQSFVHIVQMFINTSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYASTYR |
| | | VVSVLTVLHQDWLNGKEYKCKVSNKALPAPIEKTISKAKGQP |
| | | REPQVYTLPPCREEMTKNQVSLWCLVKGFYPSDIAVEWESNG |
| | | QPENNYKTTPPVLDSDGSFFLYSKLTVDKSRWQQGNVFSCSV |
| | | MHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1167 | LC CDR2 | SSSHRYK |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1173 | VL | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1310 | AA 49-162 of IL-15 | NWVNVISDLKKIEDLIQSMHIDATLYTESDVHPSCKVTAMKCF |
| | | LLELQVISLESGDASIHDTVEQLIILANNSLSSNGNVTESGCKEC |
| | | EELEEKNIKEFLQSFVHIVQMFINTS |
| SEQ ID NO: 1311 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVEQLIILANNSLSS |
| | 15Ralpha) | NGNVTESGCKECEELEEKNIKEFLQSFVHIVQMFINTS |
| SEQ ID NO: 1237 | Linker | GGGGGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0360

| | | |
|---|---|---|
| SEQ ID NO: 1289 | Heavy chain (VH- | QVQLVQSGAEVKKPGSSVKVSCKASGHDFRLTYIHWVRQAPG |
| | CH)-Hole | QGLEWMGRVSAGSGNVKYNEKFKGRVTITADTSTSTAYMELS |
| | | SLRSEDTAVYYCAVSYYSYDVLDYWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | ASTYRVVSVLTVLHQDWLNGKEYKCKVSNKALPAPIEKTISK |
| | | AKGQPREPQVCTLPPSREEMTKNQVSLSCAVKGFYPSDIAVEW |
| | | ESNGQPENNYKTTPPVLDSDGSFFLVSKLTVDKSRWQQGNVF |
| | | SCSVMHEALHNHYTQKSLSLSP |
| SEQ ID NO: 1298 | Light chain (VL-CL) | DIQMTQSPSFLSASVGDRVTITCKASQNVADRVVWHQQKPGK |
| | | APKALIYAASYLQSGVPSRFSGSGSGTEFTLTISSLQPEDFATYF |
| | | CQQFKSYPLTFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 1309 | IL-15Rαsushi-IL-15 | ITCPPPMSVEHADIWVKSYSLYSRERYICNSGFKRKAGTSSLTE |
| | Heavy Chain Constant | CVLNKATNVAHWTTPSLKCIRDPALVHQRPAPPSGGSGGGGS |
| | Domain (Fc Region)- | GGGSGGGGSGGGNWVNVISDLKKIEDLIQSMHIDATLYTESDV |
| | Knob | HPSCKVTAMKCFLLELQVISLESGDASIHDTVEQLIILANNSLSS |
| | | NGNVTESGCKECEELEEKNIKEFLQSFVHIVQMFINTSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYASTYR |
| | | VVSVLTVLHQDWLNGKEYKCKVSNKALPAPIEKTISKAKGQP |
| | | REPQVYTLPPCREEMTKNQVSLWCLVKGFYPSDIAVEWESNG |
| | | QPENNYKTTPPVLDSDGSFFLYSKLTVDKSRWQQGNVFSCSV |
| | | MHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1299 | LC CDR2 | AASYLQS |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDIAVYYCAVSYYSYDVLDYWGQGTTVTVSS |
| SEQ ID NO: 1300 | VL | DIQMTQSPSFLSASVGDRVTITCKASQNVADRVVWHQQKPGK |
| | | APKALIYAASYLQSGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 1296 | Linker | GGSGGGGSGGGSGGGGSGGG |
| SEQ ID NO: 1310 | AA 49-162 of IL-15 | NWVNVISDLKKIEDLIQSMHIDATLYTESDVHPSCKVTAMKCF |
| | | LLELQVISLESGDASIHDTVEQLIILANNSLSSNGNVTESGCKEC |
| | | EELEEKNIKEFLQSFVHIVQMFINTS |
| SEQ ID NO: 1311 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVEQLIILANNSLSS |
| | 15Ralpha) | NGNVTESGCKECEELEEKNIKEFLQSFVHIVQMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0357

| | | |
|---|---|---|
| SEQ ID NO: 1289 | Heavy chain (VH- | QVQLVQSGAEVKKPGSSVKVSCKASGHDFRLTYIHWVRQAPG |
| | CH)-Hole | QGLEWMGRVSAGSGNVKYNEKFKGRVTITADTSTSTAYMELS |
| | | SLRSEDTAVYYCAVSYYSYDVLDYWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | ASTYRVVSVLTVLHQDWLNGKEYKCKVSNKALPAPIEKTISK |
| | | AKGQPREPQVCTLPPSREEMTKNQVSLSCAVKGFYPSDIAVEW |
| | | ESNGQPENNYKTTPPVLDSDGSFFLVSKLTVDKSRWQQGNVF |
| | | SCSVMHEALHNHYTQKSLSLSP |
| SEQ ID NO: 1301 | Light chain (VL-CL) | DIQMTQSPSFLSASVGDRVTITCKASQNVADRVVWHQQKPGK |
| | | APKALIFAASYLQSGVPSRFSGSGSGTEFTLTISSLQPEDFATYF |
| | | CQQFKSYPLTFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 1309 | IL-15Rαsushi-IL-15 | ITCPPPMSVEHADIWVKSYSLYSRERYICNSGFKRKAGTSSLTE |
| | Heavy Chain Constant | CVLNKATNVAHWTTPSLKCIRDPALVHQRPAPPSGGSGGGGS |
| | Domain (Fc Region)- | GGGSGGGGSGGGNWVNVISDLKKIEDLIQSMHIDATLYTESDV |
| | Knob | HPSCKVTAMKCFLLELQVISLESGDASIHDTVEQLIILANNSLSS |
| | | NGNVTESGCKECEELEEKNIKEFLQSFVHIVQMFINTSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYASTYR |
| | | VVSVLTVLHQDWLNGKEYKCKVSNKALPAPIEKTISKAKGQP |
| | | REPQVYTLPPCREEMTKNQVSLWCLVKGFYPSDIAVEWESNG |
| | | QPENNYKTTPPVLDSDGSFFLYSKLTVDKSRWQQGNVFSCSV |
| | | MHEALHNHYTQKSLSLSPGK |
| SEQ ID NO: 1164 | HC CDR1 | GHDFRLTYIH |
| SEQ ID NO: 1229 | HC CDR1 | LTYIH |
| SEQ ID NO: 1234 | HC CDR1 | FRLTYIH |
| SEQ ID NO: 1165 | HC CDR2 | RVSAGSGNVKYNEKFKG |
| SEQ ID NO: 1005 | HC CDR3 | SYYSYDVLDY |
| SEQ ID NO: 1171 | LC CDR1 | KASQNVADRVV |
| SEQ ID NO: 1299 | LC CDR2 | AASYLQS |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWQQGTTVTVSS |
| SEQ ID NO: 1303 | VL | DIQMTQSPSFLSASVGDRVTITCKASQNVADRVVWHQQKPGK |
| | | APKALIFAASYLQSGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1295 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWITPSLKCIRDPALVHQRPAPPS |
| SEQ ID NO: 1296 | Linker | GGSGGGGSGGGSGGGGSGGG |
| SEQ ID NO: 1310 | AA 49-162 of IL-15 | NWVNVISDLKKIEDLIQSMHIDATLYTESDVHPSCKVTAMKCF |
| | | LLELQVISLESGDASIHDTVEQLIILANNSLSSNGNVTESGCKEC |
| | | EELEEKNIKEFLQSFVHIVQMFINTS |
| SEQ ID NO: 1311 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVEQLIILANNSLSS |
| | 15Ralpha) | NGNVTESGCKECEELEEKNIKEFLQSFVHIVQMFINTS |
| SEQ ID NO: 1237 | Linker | GGGGSGGGGS |
| SEQ ID NO: 1206 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BKM0186, also referred to as BK141 or BK104
Binds to human TCRVβ 6-2

| | | |
|---|---|---|
| SEQ ID NO: 1289 | Heavy chain (VH- | QVQLVQSGAEVKKPGSSVKVSCKASGHDFRLTYIHWVRQAP |
| | CH) | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 1239 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 1315 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 1164 | HC CDR1 | GHDFRLTYIH |
| SEQ ID NO: 1165 | HC CDR2 | RVSAGSGNVKYNEKFKG |
| SEQ ID NO: 1167 | HC CDR3 | SYYSYDVLDY |
| SEQ ID NO: 1170 | LC CDR1 | KASQNVADRVV |
| SEQ ID NO: 1171 | LC CDR2 | SSSHRYK |
| SEQ ID NO: 1008 | LC CDR3 | QQFKSYPLT |
| SEQ ID NO: 1168 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1173 | VL | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIK |
| SEQ ID NO: 1293 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 1294 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 1203 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 1242 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 1237 | Linker | GGGGSGGGGS |
| SEQ ID NO: 1316 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |

TABLE 34
Exemplary binding affinities of exemplary molecule
comprising an antigen binding domain that binds
to a T cell receptor beta variable region (TCRβV).

| Exemplary molecule | Affinity (K_{D}) |
|---|---|
| BMM0306 | Affinity of TCRβV arm = 1.2 nM |
| | Affinity of IL-15Rαsushi-IL-15 arm = 0.2 nM |
| BMM0354 | Affinity of TCRβV arm = 67 nM |
| | Affinity of IL-15Rαsushi-IL-15 arm = 0.2 nM |
| BMM0356 | Affinity of TCRβV arm = 681 nM |
| | Affinity of IL-15Rαsushi-IL-15 arm = 0.2 nM |
| BMM0307 | Affinity of TCRβV arm = 1.2 nM |
| | Affinity of IL-15Rαsushi-IL-15 arm = 3.2 nM |
| BMM0359 | Affinity of TCRβV arm = 67 nM |
| | Affinity of IL-15Rαsushi-IL-15 arm = 3.2 nM |
| BMM0355 | Affinity of TCRβV arm = 681 nM |
| | Affinity of IL-15Rαsushi-IL-15 arm = 3.2 nM |
| BMM0308 | Affinity of TCRβV arm = 1.2 nM |
| | Affinity of IL-15Rαsushi-IL-15 arm = 40 nM |
| BMM0360 | Affinity of TCRβV arm = 67 nM |
| | Affinity of IL-15Rαsushi-IL-15 arm = 40 nM |
| BMM0357 | Affinity of TCRβV arm = 681 nM |
| | Affinity of IL-15Rαsushi-IL-15 arm = 40 nM |

### EXAMPLES

The present disclosure will be more specifically illustrated by the following Examples. However, it should be understood that the present disclosure is not limited by these examples in any manner.

### Part I

### Example 1: Humanization of Anti-TRAV1-2, Anti-TRBV20, Anti-TRBV6, and Anti-TRBV20 and Anti-TRBV6 Antibody

The germline for the mouse anti-TRAV1-2 Antibody clone VH and VL will be assigned using IMGT nomenclature, with CDR regions defined by a combined Kabat and Chothia classification.

Humanization of the anti-TRAV1-2 Antibody VH and VL sequences will be done separately using similar methodology. Amino acids positions will be identified in the framework regions which are important for the success of CDR grafting. Human germline sequences will be identified which will preserve the necessary residues and contained a high amount of overall identity. When the human germline framework sequence does not contain a matching important amino acid, it will be back mutated to match the mouse sequence. CDR regions will be grafted onto the human germline unchanged. The anti-TRAV1-2 Antibody VH will be humanized into human IGHV1-69*01 and the anti-TRAV1-2 Antibody VL will be humanized into IGKV1-17*01 and IGKV1-27*01. All 3 humanized sequences will be confirmed to contain no introduced potential negative post translational modification sites such as NG, DG, NS, NN, DS, NT, NXS, or NXT as a result of the humanization process.

Humanization of anti-TRBV20, anti-TRBV6, and anti-TRBV20 and anti-TRBV6 Antibody will be produced in the same method as described above.

### Example 2: Expression and Purification of Antibody Constructs

### Construction of the Plasmids

The DNA encoding the protein sequences will be optimized for expression and cloned into a plasmid of choice.

### Expression and Purification

The plasmids will be co-transfected into either Expi293 cells (Life Technologies A14527) or ExpiCHO cells (Life Technologies A29127). Transfections will be performed using 1 mg of total DNA for a multispecific construct with a 1:1 heavy chain ratio and 3:2 light chain to heavy chain ratio if applicable. Transfection in Expi293 cells will be done using linear 25,000 Da polyethylenimine (PEI, Polysciences Inc 23966) in a 3:1 ratio with the total DNA. The DNA and PEI will be each added to 50 mL of OptiMem (Life Technologies 31985088) medium and sterile filtered. The DNA and PEI will be combined for 10 minutes and added to the Expi293 cells with a cell density of 1.8-2.8×106 cells/mL and a viability of at least 95%. The ExpiCHO transfection will be performed according to the manufacturer's instructions. Expi293 cells will be grown in a humidified incubator at 37° C. with 8% CO2 for 5-7 days after transfection and ExpiCHO cells will be grown for 14 days at 32° C. with 5% CO2. The cells will be pelleted by centrifugation at 4500-g and the supernatant will be filtered through a 0.2 μm membrane. Protein A resin (GE 17-1279-03) will be added to the filtered supernatant and incubated for 1-3 hours at room temperature. The resin will be packed into a column, washed with 3×10 column volumes of Dulbecco's phosphate-buffered saline (DPBS, Life Technologies 14190-144). The bound protein will be eluted from the column with 20 mM citrate, 100 mM NaCl, pH 2.9. When necessary, the proteins will be further purified using ligand affinity and/or size exclusion chromatography on a Superdex 200 column with a running buffer of DPBS.

### Example 3: Expansion of MAIT Cells Using Multispecific Molecule as Described Herein

Untouched purified naïve human MAIT cells from healthy donors will be directly procured. A 96-well plate will be coated with a multispecific molecule disclosed herein for 24 hours at 4° C. 2×10⁵ T cells will be seeded into each well and for incubated at 37° C. for 5 days. After the 5-day culture, cell pellets will be washed with FACS buffer (0.1% BSA, PBS) and stained with Live/Dead Zombie-UV, PerCPcy5.5/anti-CD3, BV421/anti-CD4, BV605/anti-CD8 (Biolegend) and secondary antibody recognizing the multispecific molecule for 20 mins. After FACS-antibody staining, cells will be washed with FACS buffer and ran on flow cytometry analyzer, Cytoflex (Beckman Coulter) for data collection. FACS plots will be used to represent MAIT cells gated on CD3+ T-cells for MAIT cell expansion (FIG. 2A). Unstimulated T-cells serves as baseline levels.

### Example 4: Activation of MAIT Cells Using Multispecific Molecule as Described Herein

Human peripheral blood mononuclear cells (PBMCs) will be thawed and plated at 3×10 cells/well in a 96-well flat-bottom plate with 0.001, 0.01, 0.1, 1, 10, or 100 nM of a multispecific molecule disclosed herein for 5 days at 37° C. After the 5-day culture, cell pellets were washed with FACS buffer (0.1% BSA, PBS) and stained with anti-CD4, anti-CD25 (IL2RA), and anti-CD8 antibodies for 20 mins. After FACS-antibody staining, cells will be washed with FACS buffer and ran on flow cytometry analyzer, Cytoflex (Beckman Coulter) for data collection. Dot plots represent expansion and activation of MAIT cells in treated cells compared to isotype controls.

### Example 5: Therapeutic Efficacy of Multispecific Molecule as Described Herein in Human Tumor Xenograft Models

Spleens from two naïve BALB/c or C57BL/6 albino mice will be mechanically dissociated with the backend of syringe through a 70 μM cell strainer in RPMI to obtain single-cell suspensions. Cells will be centrifuged at 300G for 10 mins and the supernatant will be removed. Red blood cells will be lysed by addition of 1 mL ACK lysis buffer to the pellet. Well-resuspended cells will be incubated for 3 mins on ice. 10 mL RPMI will be added, and cells will be centrifuged at 300G for 10 mins. Cell pellet will be resuspended in splenocyte media (RPMI+10% FBS+L-Glut+Pen/Strep+1.5 mM HEPES+0.5 mM BME), then filtered with a 70 μM cell strainer. MAIT cells will be isolated using T Cell Isolation Kit 11, mouse (Miltenyi Biotec cat #130-095-130). Isolated MAIT cells were plated at 0.001, 0.01, 0.1, 1, 10, or 100 nM of multispecific molecule as described herein with for 4 days at 37° C. After the 4-day culture, cell pellets will be washed with FACS buffer (0.1% BSA, PBS) and stained with anti-CD4, anti-CD25 (IL2RA), and anti-CD8 antibodies for 20 mins. After FACS-antibody staining, cells will be washed with FACS buffer and ran on flow cytometry analyzer, Cytoflex (Beckman Coulter) for data collection. Dot plots will represent expansion and activation of MAIT cells in treated cells compared to isotype controls.

### Part II

### Example 6: Generation of Non-Human Anti-TRAV10 and Anti-TRBV25 Antibody

Preparation of Polyclonal Antisera and Immunogens. Methods of generating antibodies or antibody fragments of the present technology typically include immunizing a subject (generally a non-human subject such as a mouse or rabbit) with a purified hTRAV10 or hTRBV25 protein or fragment thereof, a nucleic acid encoding hTRAV10 or hTRBV25protein or fragment thereof, or with a cell expressing the hTRAV10 or hTRBV25 protein or fragment thereof. An appropriate immunogenic preparation can contain, e.g., a recombinantly-expressed hTRAV10 or hTRBV25 protein or a chemically-synthesized hTRAV10 or hTRBV25 peptide. In some embodiments, the antigenic hTRAV10 or hTRBV25 peptide comprises at least 10, at least 20, at least 30, at least 40, at least 50, at least 60, at least 70, at least 80, at least 90, or at least 100 amino acid residues. Longer antigenic peptides are sometimes desirable over shorter antigenic peptides, depending on use and according to methods well known to those skilled in the art. Multimers of a given epitope are sometimes more effective than a monomer.

If needed, the immunogenicity of the hTRAV10 or hTRBV25 protein (or fragment thereof) can be increased by fusion or conjugation to a carrier protein such as keyhole limpet hemocyanin (KLH) or ovalbumin (OVA). Many such carrier proteins are known in the art. One can also combine the hTRAV10 or hTRBV25 protein with a conventional adjuvant such as Freund's complete or incomplete adjuvant to increase the subject's immune reaction to the polypeptide. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory compounds. These techniques are standard in the art.

Alternatively, nanoparticles, for example, virus-like particles (VLPs), can be used to present antigens, e.g., hTRAV10 or hTRBV25, to a host animal. Virus-like particles are multiprotein structures that mimic the organization and conformation of authentic native viruses without being infectious, since they do not carry any viral genetic material (Urakami A, et al, Clin Vaccine Immunol 24: e00090-17 (2017)). When introduced to a host immune system. VLPs can evoke effective immune responses, making them attractive carriers of foreign antigens. An important advantage of a VLP-based antigen presenting platform is that it can display antigens in a dense, repetitive manner. Thus, antigen-bearing VLPs are able to induce strong B-cell responses by effectively enabling the cross-linking of B cell receptors (BCRs). VLPs may be genetically manipulated to fine their properties, e.g., immunogenicity. These techniques are standard in the art.

Nucleic acid (DNA and RNA) based immunizations have emerged as promising alternatives. DNA vaccines are usually based on bacterial plasmids that encode the polypeptide sequence of candidate antigen, e.g., hTRAV10 or hTRBV25. With a robust eukaryotic promoter, the encoded antigen can be expressed to yield enough levels of transgene expression once the host is inoculated with the plasmids (Galvin T. A., et al., Vaccine 2000, 18:2566-2583). Modem DNA vaccine generation relies on DNA synthesis, possibly one-step cloning into the plasmid vector and subsequent isolation of the plasmid, which takes significantly less time and cost to manufacture. The resulting plasmid DNA is also highly stable at room temperature, avoiding cold transportation and leading to substantially extended shelf-life. These techniques are standard in the art.

Alternatively, nucleic acid sequences encoding the antigen of interest, e.g., hTRAV10 or hTRBV25, can be synthetically introduced into a mRNA molecule. The mRNA is then delivered into a host animal, whose cells would recognize and translate the mRNA sequence to the polypeptide sequence of the candidate antigen, e.g., hTRAV10 or hTRBV25, thus triggering the immune response to the foreign antigen. An attractive feature of mRNA antigen or mRNA vaccine is that mRNA is a non-infectious, non-integrating platform. There is no potential risk of infection or insertional mutagenesis associated with DNA vaccines. In addition, mRNA is degraded by normal cellular processes and has a controllable in vivo half-life through modification of design and delivery methods. These techniques are standard in the art.

In describing the present technology, immune responses may be described as either “primary” or “secondary” immune responses. A primary immune response, which is also described as a “protective” immune response, refers to an immune response produced in an individual as a result of some initial exposure (e.g., the initial “immunization” or “priming”) to a particular antigen, e.g., hTRAV10 or hTRBV25 protein. A primary immune response can become weakened or attenuated over time and can even disappear or at least become so attenuated that it cannot be detected. Accordingly, the present technology also relates to a “secondary” immune response, which is also described here as a “memory immune response.” The term secondary immune response refers to an immune response elicited in an individual after a primary immune response has already been produced.

Thus, a secondary immune response can be elicited, e.g., to enhance an existing immune response that has become weakened or attenuated (e.g., boosting), or to recreate a previous immune response that has either disappeared or can no longer be detected. The secondary or memory immune response can be either a humoral (antibody) response or a cellular response. A secondary or memory humoral response occurs upon stimulation of memory B cells that were generated at the first presentation of the antigen. Delayed type hypersensitivity (DTH) reactions are a type of cellular secondary or memory immune response that are mediated by CD4⁺ T cells. A first exposure to an antigen primes the immune system and additional exposure(s) results in a DTH.

Following appropriate immunization, the anti-TRAV10 or anti-TRBV25 antibody can be prepared from the subject's serum. If desired, the antibody molecules directed against the TRAV10 or TRBV25 protein can be isolated from the mammal (e.g., from the blood) and further purified by well-known techniques, such as polypeptide A chromatography to obtain the IgG fraction.

### Example 7: Humanization of Non-Human Anti-TRAV10 and Non-Human Anti-TRBV25 Antibody

The germline for the non-human TRAV10 (or TRBV25) antibody clone VH and VL will be assigned using IMGT nomenclature, with CDR regions defined by a combined Kabat and Chothia classification. Humanization of the VH and VL sequences will be done separately using similar methodology.

Amino acids positions will be identified in the framework regions which are important for the success of CDR grafting. Human germline sequences will be identified which preserved the necessary residues and contained a high amount of overall identity. When the human germline framework sequence does not contain a matching important amino acid, it will be back mutated to match the non-human (e.g., mouse sequence). CDR regions will be grafted onto the human germline unchanged. The VH will be humanized into the corresponding human germline identified above, and the VL will be humanized into corresponding human germline identified above. All humanized sequences will be confirmed to contain no introduced potential negative post translational modification sites such as NG, DG, NS, NN, DS, NT, NXS, or NXT as a result of the humanization process

### Example 8: Expression and Purification of Multispecific Antibodies of the Present Disclosure

### Construction of the Plasmids

The DNAs encoding the protein sequences will be optimized for expression and cloned into the suitable plasmids.

### Expression and Purification

The plasmids are (co-)transfected into suitable expressing cells. Transfections will be performed using known methods. For a non-limiting example, transfection in the cells may be done using linear 25,000 Da polyethylenimine (PEI, Polysciences Inc 23966) in a 3:1 ratio with the total DNA. The DNA and PEI will each added to 50 mL of OptiMem (Life Technologies 31985088) medium and sterile filtered. The DNA and PEI will be combined for 10 minutes and added to the Expi293 cells with a cell density of 1.8-2.8×10⁶ cells/mL and a viability of at least 95%. The cells will be grown in a humidified incubator at 37° C. with 8% CO₂ for 5-7 days after transfection. The cells will be pelleted by centrifugation at 4500×g and the supernatant will be filtered through a 0.2 μm membrane. Protein A resin (GE 17-1279-03) will be added to the filtered supernatant and incubated for 1-3 hours at room temperature. The resin will be packed into a column, washed with 3×10 column volumes of Dulbecco's phosphate-buffered saline (DPBS, Life Technologies 14190-144). The bound protein will be eluted from the column with 20 mM citrate, 100 mM NaCl, pH 2.9. When necessary, the proteins will be further purified using ligand affinity and/or size exclusion chromatography on a Superdex 200 column with a running buffer of DPBS.

### Example 9: Antibody-Antigen Interaction & Binding Kinetics Analysis Via Plasmon Resonance (SPR) Binding

All interactions between multispecific antibodies of the present disclosure and relevant receptors may be analyzed by Surface Plasmon Resonance (SPR) on a Biacore T200 instrument. An exemplary operation protocol is as the following.

An multispecific antibody of the present disclosure will be immobilized on a Series S CM5 chip via human Fc antibody to 80 RU. Corresponding antigen (e.g., Human TRAV10 or TRBV25) will be diluted (e.g., to 500 nM) and then serially diluted two-fold in 1×HBS-EP+ buffer for a total of 10 concentrations. Multi cycle kinetics was run where the chip is regenerated with 3 M Magnesium Chloride between each cycle followed by a new injection of the multispecific antibody. The association time seconds and dissociation time will be measured at 30 uL/min for suitable time. This assay will be run in 1×HBS-EP+ Buffer pH 7.4 and 25° C. Sensorgrams will be corrected by double reference subtraction using the reference flow cell not treated with the multispecific antibody and a blank cycle of buffer alone. BIAevaluation software will be used for data analysis and the data will be fit using a 1:1 Langmuir binding model for calculation of the KD value (Koff/Kon).

### Example 10: Activation and/or Expansion of iNKT by the Multispecific Antibody of the Present Disclosure

Human PBMCs and/or isolated iNKT cells will be treated by the multispecific antibody of the present disclosure. Cytokine and chemokine profile, and kinetics of iNKT cell expansion will be evaluated.

### Part III

### Example 11. Humanization of α-TRBV6-2 Antibody Clone Antibody A

The germline for the mouse α-TCRβ antibody clone Antibody A VH and VL are assigned using IMGT nomenclature, with CDR regions defined by a combined Kabat and Chothia classification. SEQ ID NO: 1001 and SEQ ID NO: 1002 are the Antibody A VH and VL sequences respectively where the VH germline is mouse IGHV1S12*01 and the VL germline is mouse IGKV6-15*01. SEQ ID NOs: 1003-1005 are the Antibody A VH CDR regions 1-3 respectively and SEQ ID NOs: 1006-1008 correspond to the VL CDR regions 1-3 (as described in Table 21).

Humanization of the Antibody A VH and VL sequences is done separately using similar methodology. Amino acids positions are identified in the framework regions which are important for the success of CDR grafting. Human germline sequences are identified which preserved the necessary residues and contained a high amount of overall identity. When the human germline framework sequence does not contain a matching important amino acid, it is back mutated to match the mouse sequence. CDR regions are grafted onto the human germline unchanged. The Antibody A VH is humanized into human IGHV1-69*01 and the Antibody A VL is humanized into IGKV1-17*01 and IGKV1-27*01. All 3 humanized sequences will be confirmed to contain no introduced potential negative post translational modification sites such as NG, DG, NS, NN, DS, NT, NXS, or NXT as a result of the humanization process. SEQ ID NO: 1009 is the humanized Antibody A-H.1 VH and SEQ ID NOs: 1010 and 1011 are the humanized VL IGKV1-17*01 and IGKV1-27*01 germlines respectively (as described in Table 21).

Similar methodology is repeated for humanization of anti-TRBV4-1, anti-TRAV1-2, and anti-TRAV17 antibodies.

### Example 12: Expression and Purification of Antibody Constructs

### Construction of the Plasmids

The DNA encoding the protein sequences is optimized for expression in Cricetulus griseus, synthesized, and cloned into the pcDNA3.4-TOPO (Life Technologies A14697) using Gateway cloning. All constructs contain an Ig Kappa leader sequence METDTLLLWVLLLWVPGSTG (SEQ ID NO: 1290).

### Expression and Purification

The plasmids are co-transfected into either Expi293 cells (Life Technologies A14527) or ExpiCHO cells (Life Technologies A29127). Transfections are performed using 1 mg of total DNA for a multispecific construct with a 1:1 heavy chain ratio and 3:2 light chain to heavy chain ratio if applicable. Transfection in Expi293 cells is done using linear 25,000 Da polyethylenimine (PEI, Polysciences Inc 23966) in a 3:1 ratio with the total DNA. The DNA and PEI are each added to 50 mL of OptiMem (Life Technologies 31985088) medium and sterile filtered. The DNA and PEI are combined for 10 minutes and added to the Expi293 cells with a cell density of 1.8-2.8×10⁶ cells/mL and a viability of at least 95%. The ExpiCHO transfection is performed according to the manufacturer's instructions. Expi293 cells are grown in a humidified incubator at 37° C. with 8% CO₂ for 5-7 days after transfection and ExpiCHO cells are grown for 14 days at 32° C. with 5% CO₂. The cells are pelleted by centrifugation at 4500×g and the supernatant is filtered through a 0.2 μm membrane. Protein A resin (GE 17-1279-03) is added to the filtered supernatant and incubated for 1-3 hours at room temperature. The resin is packed into a column, washed with 3×10 column volumes of Dulbecco's phosphate-buffered saline (DPBS, Life Technologies 14190-144). The bound protein is eluted from the column with 20 mM citrate, 100 mM NaCl, pH 2.9. When necessary, the proteins are further purified using ligand affinity and/or size exclusion chromatography on a Superdex 200 column with a running buffer of DPBS.

### Example 13: Kinetics of T Cell Expansion Following TCRβV6-2 Stimulation

To assess the kinetics and absolute count of anti-TCRβv 6-2 expanded T cells—either PBMCs or purified T cells were stimulated with plate-immobilized anti-TCRvb 6-2 antibody over 8 days with a T cell-activating antibody at 100 nM. T cell activating antibodies tested included: i) anti-TCRvb 6-2 antibody; ii) OKT3 (anti-CD3ε antibody); iii) SP34-2 (anti-CD3ε antibody); and iv) IgGI N297A (isotype control). Cell pellets are collected each day and stained for CD3, CD4, CD8 and TCRvb 6-5 for flow analysis.

TCRvb 6-2+ T cells are expanded over 8 days using anti-TCRvb 6-2, assessed by flow cytometry. Three independent donors are used. Specific expansion of TCRvb 6-2+CD4+ T cells and TCRvb 6-2+CD8+ T cells by TCRvb 6-2 are assessed. Expansion of TCRvb 6-2+ T-cells by anti-TCRvb 6-2 is compared to those by OKT3.

Similar methodology is repeated for humanization of anti-TRBV4-1, anti-TRAV1-2, and anti-TRAV17 antibodies.

### Example 14: Characteristics of Anti-TCRβV Antibody a Antibody

This Example describes characterization of anti-TCRβV antibodies which can bind and activate a subset of T cells, but with, e.g., no or markedly reduced, CRS.

Human PBMCs are isolated from whole blood followed by solid-phase (plate-coated) stimulation with anti-TCR Vβ6 antibody (Antibody A) or anti-CD3e antibodies (OKT3) at 100 nM. Supernatant is collected on Days 1, 2, 3, 5, or 6 followed by multiplex cytokine analysis for IFNg, IL-2, IL-6, IL-1beta, or TNFalpha. The data is quantified using MSD (Meso Scale Discovery) platform, following the manufacturer's protocol.

When plate-bound anti-TCR Vβ6 antibody (Antibody A) or anti-CD3e antibodies (OKT3) are used to activate human PBMCs, the T cell cytokine IFNg is induced. With respect to IL-2 production, PBMCs activated with anti-TCR Vβ12 antibody (Antibody D) results in increased IL-2 production with delayed kinetics as compared to PBMCs activated with anti-CD3e antibodies (OKT3).

The production of cytokines IL-6, IL-10 and TNF-alpha which are associated with “cytokine storms” (and accordingly CRS) is also assessed under similar conditions. PBMCs activated with anti-CD3e antibodies demonstrate production of IL-6, TNF-alpha, and IL-1β, no or little induction of these cytokines is observed with PBMCs activated with anti-TCR Vβ6 antibody (Antibody A).

Similar assays are repeated for anti-TRBV4-1, anti-TRAV1-2, and anti-TRAV17 antibodies.

The data provided in this Example show that antibodies directed against TCR VP can, e.g., preferentially activate a subset of T cells, and do not result in induction of cytokines associated with cytokine storms or CRS.

### Example 15: Cytokine and Chemokine Profile of Anti-TCRVb Antibodies

This Examples describes cytokines and chemokines secreted by PBMCs following activation by anti-TCR VP antibodies.

Human PBMCs are isolated from whole blood followed by solid-phase (plate-coated) stimulation with an anti-TCRβV6 antibodies (A-H.1), an isotype control (BGM0122) or an anti-CD3e antibody (SP34), each at 100 nM. Supernatant is collected on Days 1, 2, 3, 4, 5, 6, 7 and 8 followed by multiplex analysis for the indicated cytokines or chemokines. The data is quantified using MSD (Meso Scale Discovery) platform, following the manufacturer's protocol. BGM0122 comprises the amino acid sequence of

(SEQ ID NO: 1291)
METDTLLLWVLLLWVPGSTGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSP
GKGGGGSGGGGSGLNDIFEAQKIEWHE.

Plate-bound anti-TCR Vβ6 antibodies or anti-CD3e antibodies (OKT3) are used to activate human PBMCs, the T cell cytokine IFNg is induced. With respect to IL-2 production, PBMCs activated with anti-TCR Vβ antibodies results in increased IL-2 production with delayed kinetics as compared to PBMCs activated with anti-CD3e antibody (OKT3).

While IL-1beta, IL-6, IL-10, IL-4, TNFalpha, IP-10, IL-12-23p40, IL-17A, and IL-1a, are induced by anti-CD3e antibody (OKT3), no or little induction of these cytokines or chemokines is produced in PBMCs activated with anti-TCRVb antibodies.

Similar assays are repeated for anti-TRBV4-1, anti-TRAV1-2, and anti-TRAV17 antibodies.

### Example 16: Nanostring-Based Gene Expression Profiling of TCR Vb-Activated T Cells

This Example describes gene expression profiling of TCR Vβ-activated T cells to, e.g., uncover potential mechanisms or pathways underlying TCR Vβactivation of T cells.

In a first study, the anti-TCR V06 antibody A-H.1 is compared with an anti-CD3 antibody OKT3. Human PBMCs are isolated from whole blood. From isolated PBMCs, human CD3+ T cells are isolated using magnetic-bead separation (negative selection) (Miltenyi Biotec) and activated by immobilized (plate-coated) anti-TCR V06 antibody (A-H. 1) or anti-CD3 antibody (OKT3) at 100 nM for 6 days. Activated T-cells (from plate-coated) are then prepared for gene expression profiling (PanCancer IO 360™ Panel, nanoString), following manufacturer's protocol. Differential gene expression analysis is grouped by anti-TCR V06 (A-H.1) vs anti-CD3 (OKT3) activated T-cells using the nSolver Analysis Software (Nanostring). The differentially regulated genes are filtered by having a p-value of 0.05 or less. A positive value of fold change in gene expression indicates genes that are upregulated at the transcriptional level in TCR Vβ-activated T cells compared to OKT3-activated T cells, whereas a negative value of fold change in gene expression indicates genes downregulated at the transcriptional level in TCR Vβ-activated T cells compared to OKT3-activated T cells.

Similar assays are repeated for anti-TRBV4-1, anti-TRAV1-2, and anti-TRAV17 antibodies.

### Example 17: Expansion of T-Cells Using an Anti-TRAV or Anti-TRBV Antibody

Untouched purified naïve human T-cells from healthy donors are directly procured. A 96-well plate is coated with an anti-TCR V alpha 1-2 for 24 hours at 4° C. 2×10 WT cells are seeded into each well and incubated at 37° C. for 5 days. After the 5-day culture, cell pellets are washed with FACS buffer (0.1% BSA, PBS) and stained with Live/Dead Zombie-UV, PerCPcy5.5/anti-CD3, BV421/anti-CD4, BV605/anti-CD8 (Biolegend) and anti-TCR V alpha 1-2 labeled using the Alexa Fluor™ 647 NHS Ester (ThermoFisher) for 20 mins. After FACS-antibody staining, cells are washed with FACS buffer and run on flow cytometry analyzer, Cytoflex (Beckman Coulter) for data collection. CD3+ T-cells are quantified to determine T-cell TCR V alpha 1-2 expansion. Unstimulated T-cells serves as baseline TCR V alpha 1-2 levels. Similar assays are repeated to confirm expansion of T-cells using an anti-TCR V alpha 17, anti-TCR V beta 4-1, and anti-TCR V beta 6-2 antibodies.

### Example 18: Expansion of Cd1b/c Restricted T-Cells Using an Anti-TRAV or Anti-TRBV Antibody

Untouched purified naïve human T-cells from healthy donors are directly procured. A 96-well plate is coated with an anti-TCR V alpha 1-2 for 24 hours at 4° C. 2×10 T cells are seeded into each well and incubated at 37° C. for 5 days. After the 5-day culture, cell pellets are washed with FACS buffer (0.1% BSA, PBS) and stained with Live/Dead Zombie-UV, PerCPcy5.5/anti-CD3, PE/anti-CD1b, BV605/anti-CD1c (Biolegend) and anti-TCR V alpha 1-2 labeled using the Alexa Fluor™ 647 NHS Ester (ThermoFisher) for 20 mins. After FACS-antibody staining, cells are washed with FACS buffer and run on flow cytometry analyzer, Cytoflex (Beckman Coulter) for data collection. CD1b+/CD1c+ T-cells are quantified to determine CD1b+/CD1c+ T-cells for TCR V alpha 1-2 expansion. Unstimulated T-cells serves as baseline TCR V alpha 1-2 levels. Similar assays are repeated to confirm expansion of CD1b/c T-cells using an anti-TCR V alpha 17, anti-TCR V beta 4-1, and anti-TCR V beta 6-2 antibodies.

### Example 19: Expansion of T-Cells Using an Anti-TRAV or Anti-TRBV Antibody and IL-2

Untouched purified naïve human T-cells from healthy donors are directly procured. A 96-well plate is coated with 100 nM of anti-TCR V alpha 1-2 for 24 hours at 4° C. 2×10⁵ T cells are seeded into each well and for incubated at 37° C. for 10 days; followed by change to fresh media containing 100 U/mL of recombinant human IL-2 and continued incubation for additional 2 days to allow further expansion of activated TCR V alpha 1-2 T-cells. After the 2-day culture, cell pellets are washed with FACS buffer (0.1% BSA, PBS) and stained with Live/Dead Zombie-UV, PerCPcy5.5/anti-CD3, BV421/anti-CD4, BV605/anti-CD8 (Biolegend) and anti-TCR V alpha 1-2 labeled using the Alexa Fluor™ 647 NHS Ester (ThermoFisher) for 20 mins. After FACS-antibody staining, cells are washed with FACS buffer and ran on flow cytometry analyzer, Cytoflex (Beckman Coulter) for data collection. CD3+ T-cells are quantified to determine T-cell TCR V alpha 1-2 expansion. Unstimulated T-cells serves as baseline TCR V alpha 1-2 levels. Similar assays are repeated to confirm expansion of T-cells using an anti-TCR V alpha 17, anti-TCR V beta 4-1, and anti-TCR V beta 6-2 antibodies.

### Example 20: Expansion of CD1b/c-Restricted T-Cells Using an Anti-TRAV or Anti-TRBV Antibody and IL-2

Untouched purified naïve human T-cells from healthy donors are directly procured. A 96-well plate was coated with 100 nM of anti-TCR V alpha 1-2 for 24 hours at 4° C. 2×10⁵ T cells are seeded into each well and for incubated at 37° C. for 10 days; followed by change to fresh media containing 100 U/mL of recombinant human IL-2 and continued incubation for additional 2 days to allow further expansion of activated TCR V alpha 1-2 T-cells. After the 2-day culture, cell pellets are washed with FACS buffer (0.1% BSA, PBS) and stained with Live/Dead Zombie-UV, PE/anti-CD1b, BV605/anti-CD1c (Biolegend) and anti-TCR V alpha 1-2 labeled using the Alexa Fluor™ 647 NHS Ester (ThermoFisher) for 20 mins. After FACS-antibody staining, cells are washed with FACS buffer and ran on flow cytometry analyzer, Cytoflex (Beckman Coulter) for data collection. T-cells gated on CD1b+/CD1c+ T-cells are quantified to determine CD1b+/CD1c+ T-cells for TCR V alpha 1-2 expansion. Unstimulated T-cells serves as baseline TCR V alpha 1-2 levels. Similar assays are repeated to confirm expansion of CD1b/c T-cells using an anti-TCR V alpha 17, anti-TCR V beta 4-1, and anti-TCR V beta 6-2 antibodies.

### Example 21: Expansion of Human T-Cells Using an Anti-TCRV/IL-2 or Anti-TCRβV/IL-2 Bispecific Antibody

Human peripheral blood mononuclear cells (PBMCs) are thawed and plated at 3×10⁵ cells/well in a 96-well flat-bottom plate with 0.001, 0.01, 0.1, 1, 10, or 100 nM of bispecific antibody molecule with specificity for human TCRαV-1-2 and IL-2 (anti-TCRαV-1-2/IL-2) for 5 days at 37° C. After the 5-day culture, cell pellets are washed with FACS buffer (0.1% BSA, PBS) and stained with anti-CD4, anti-CD25 (IL2RA), and anti-CD8 antibodies for 20 mins. After FACS-antibody staining, cells are washed with FACS buffer and ran on flow cytometry analyzer, Cytoflex (Beckman Coulter) for data collection. CD4+CD25+ and CD8+CD25+ T-cells in anti-TCRαV-19/IL-2 treated cells are quantified compared to isotype controls to determine expansion of T-cells. Similar assays are performed with anti-TCRαV-17/IL-2, anti-TCRβV-4-1/IL2, and anti-TCRβV-6-/IL2 antibodies.

### Example 22: Expansion of Human CD1b/c Restricted T-Cells Using an Anti-TCRαV/IL-2 or Anti-TCRβV/IL-2 Bispecific Antibody

Human peripheral blood mononuclear cells (PBMCs) are thawed and plated at 3×10⁵ cells/well in a 96-well flat-bottom plate with 0.001, 0.01, 0.1, 1, 10, or 100 nM of bispecific antibody molecule with specificity for human TCRαV-1-2 and IL-2 (anti-TCRαV-1-2/IL-2) for 5 days at 37° C. After the 5-day culture, cell pellets are washed with FACS buffer (0.1% BSA, PBS) and stained with, anti-CD25 (IL2RA), anti-CD1b and anti-CD1c antibodies for 20 mins. After FACS-antibody staining, cells are washed with FACS buffer and ran on flow cytometry analyzer, Cytoflex (Beckman Coulter) for data collection. CD41b+CD25+ and CD1c+CD25+ T-cells in anti-TCRαV-19/IL-2 treated cells are quantified compared to isotype controls to determine expansion of T-cells. Similar assays are performed with anti-TCRαV-17/IL-2, anti-TCRβV-4-1/IL2, and anti-TCRβV-6-/IL2 antibodies.

1.-410. (canceled)

411. A method of treating a disease or condition in a subject in need thereof comprising: administering to the subject a therapeutically effective amount of a composition comprising a molecule, wherein the molecule comprises:
(i) a first domain that binds to a T cell receptor alpha variable 1-2 (TRAV1-2), and
(ii) a second domain comprising a cytokine or a functional fragment or variant thereof.

412. The method of claim 411, wherein the cytokine comprises IL-2, IL-7, IL-12, IL-15, IL-18, IL-21, or any functional fragment or variant thereof.

413. The method of claim 411, wherein the molecule further comprises a third domain comprising an antigen binding domain.

414. The method of claim 413, wherein the antigen binding domain binds to a tumor associated antigen (TAA).

415. The method of claim 414, wherein the TAA is a tumor antigen, a stromal antigen, or a hematological antigen.

416. The method of claim 415, wherein the TAA is selected from the group consisting of CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYPIB1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), and immunoglobulin lambda-like polypeptide 1 (IGLL1).

417. The method of claim 411, wherein the first domain binds to a MAIT cell.

418. The method of claim 417, wherein the first domain activates the MAIT cell.

419. The method of claim 411, wherein the first domain comprises a Fab, a F(ab′)2, an Fv, a single chain Fv (scFv), a single domain antibody, a diabody (dAb), a camelid antibody, or a combination thereof.

420. The method of claim 411, wherein the first domain comprises a heavy chain variable region (VH) comprising a heavy chain complementarity determining region 1 (HC CDR1) sequence, a heavy chain complementarity determining region 2 (HC CDR2) sequence, and a heavy chain complementarity determining region 3 (HC CDR3) sequence comprising the sequences of: SEQ ID NO: 197, SEQ ID NO: 198, and SEQ ID NO: 199, respectively; and a light chain variable region (VL) comprising a light chain complementarity determining region 1 (LC CDR1) sequence, a light chain complementarity determining region 2 (LC CDR2) sequence, and a light chain complementarity determining region 3 (LC CDR3) sequence comprising the sequences of: SEQ ID NO: 200, SEQ ID NO: 201, and SEQ ID NO: 202, respectively.

421. The method of claim 420, wherein the first domain comprises a VH comprising at least 90% sequence identity to an amino acid sequence as set forth in SEQ ID NO: 195, and a VL comprising at least 90% sequence identity to an amino acid sequence as set forth in SEQ ID NO: 196.

422. The method of claim 421, wherein the first domain comprises a VH comprising an amino acid sequence as set forth in SEQ ID NO: 195, and a VL comprising an amino acid sequence as set forth in SEQ ID NO: 196.

423. The method of claim 411, wherein the disease or condition is a cancer, an autoimmune disease, or an infection.

424. The method of claim 423, wherein the disease or condition is the cancer, and wherein the cancer is a solid tumor, a hematological cancer, a metastatic cancer, a soft tissue tumor, or any combination thereof.

425. The method of claim 424, wherein the cancer is the solid tumor, and wherein the solid tumor is selected from the group consisting of melanoma, pancreatic cancer, breast cancer, colorectal cancer, lung cancer, skin cancer, ovarian cancer, liver cancer, and any combination thereof.

426. The method of claim 424, wherein the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof.

427. The method of claim 423, wherein the disease or condition is the autoimmune disease, and wherein the autoimmune diseases is selected from the group consisting of multiple sclerosis, Crohn's disease, Sjögren's syndrome, celiac disease, diabetes, arthritis, and Lupus, systemic sclerosis, osteopetrosis, inherited metabolic disorders, adrenoleukodystrophy, amegakaryocytic thrombocytopenia, sickle cell disease, severe congenital immunodeficiency, Griscelli syndrome type IL, Hurler syndrome, Kostmann syndrome, Krabbe disease, metachromatic leukodystrophy, thalassemia, hemophagocytic lymphohistiocytosis, and Wiskott-Aldrich syndrome.

428. The method of claim 423, wherein the disease or condition is the infection, and wherein the infection is selected from the group consisting of a S. parathyphi infection, a Bacteroidetes infection, a Proteobacteria infection, a M. tuberculosis infection, a respiratory virus infection, a SARS-CoV-2 infection, yellow fever, influenza, a HIV infection, a Cytomegalovirus infection, and an Epstein-Barr Virus infection.

429. The method of claim 411, wherein the molecule comprises a first polypeptide and a second polypeptide, wherein the first polypeptide and the second polypeptide are non-contiguous, wherein:
(i) the first polypeptide comprises a first portion of a dimerization module linked to
(a) the first domain that binds to the TRAV1-2, or
(b) a first portion of the first domain that binds to the TRAV1-2, wherein the first portion of the first domain that binds to the TRAV1-2 comprises a heavy chain variable domain (VH),
wherein when the first polypeptide comprises the first portion of the first domain that binds to the TRAV1-2, the molecule further comprises a third polypeptide comprising a second portion of the first domain that binds to the TRAV1-2, wherein the second portion of the first domain that binds to the TRAV1-2 comprises a light chain variable domain (VL), wherein the third polypeptide is non-contiguous with the first polypeptide and the second polypeptide; and
(ii) the second polypeptide comprises a second portion of the dimerization module, wherein the cytokine or functional fragment or variant thereof is covalently linked to the first polypeptide, the second polypeptide, or the third polypeptide.

430. A multispecific molecule comprising:
(i) a first polypeptide comprising a first portion of a first domain that binds to a T cell receptor beta variable 20-1 (TCRVβ 20-1) comprising a heavy chain variable domain (VH) comprising an amino acid sequence as set forth in SEQ ID NO: 350 or 345;
(ii) a second polypeptide comprising (1) an amino acid sequence as set forth in SEQ ID NO: 1237, and (2) an amino acid sequence as set forth in SEQ ID NO: 1316; and
(iii) a third polypeptide comprising a second portion of the first domain that binds to the TCRVβ 20-1 comprising a light chain variable domain (VL) comprising an amino acid sequence as set forth in SEQ ID NO: 351 or 346.
